(19) United States
(12) Patent Application Publication
(10) Pub. No.: US 2023/0083885 A1
(43) Pub. Date: Mar. 16, 2023

(54) MODULATORS OF INTEGRATED STRESS RESPONSE PATHWAY

(71) Applicant: Praxis Biotech LLC, San Francisco, CA (US)

(72) Inventors: Gonzalo Andrés URETA DÍAZ, Santiago (CL); Brahmam PUJALA, Greater Noida (IN); Dayanand PANPATIL, Noida (IN); Sebastian BERNALES, Piedmont, CA (US); Sarvajit CHAKRAVARTY, Edmond, OK (US)

(21) Appl. No.: 17/711,961

(22) Filed: Apr. 1, 2022

Related U.S. Application Data
(60) Provisional application No. 63/170,216, filed on Apr. 2, 2021.

(51) Int. Cl. A61K 31/4709; C07K 14/535; C07K 14/50; C07D 413/12; C07D 271/06; C07D 413/04; C07D 285/08; C07D 263/32; C07D 231/12; C07D 417/12; C07D 413/14; C07D 401/12; A61K 31/4245; A61K 31/538; A61K 31/497; A61K 31/433; A61K 31/421; A61K 31/415; A01N 43/82; A01N 43/84; A01N 43/56; A01P 21/00; C12P 21/00; C12N 5/071; C12N 15/86

(52) U.S. Cl. CPC A61K 31/4709; C07K 14/535; C07K 14/50; C07D 413/12; C07D 271/06; C07D 413/04; C07D 285/08; C07D 263/32; C07D 231/12; C07D 417/12; C07D 413/14; C07D 401/12; A61K 31/4245; A61K 31/538; A61K 31/497; A61K 31/433; A61K 31/421; A61K 31/415; A01N 43/82; A01N 43/84; A01N 43/56; A01P 21/00; C12P 21/005; C12N 5/0682; C12N 15/86; C12N 2500/02; C12N 2500/32; C12N 2740/15043; C12N 2501/999

(57) ABSTRACT

The present disclosure relates generally to therapeutic agents that may be useful as modulators of Integrated Stress Response (ISR) pathway.

### CROSS REFERENCE TO RELATED APPLICATIONS

This application claims the priority benefit of U.S. Provisional Patent Application No. 63/170,216, filed Apr. 2, 2021, the disclosure of which is hereby incorporated herein by reference in its entirety.

### FIELD

The present disclosure relates generally to therapeutic agents that may be useful as modulators of Integrated Stress Response (ISR) pathway.

### BACKGROUND

Diverse cellular conditions and stresses activate a widely conserved signaling pathway termed the Integrated Stress Response (ISR) pathway. The ISR pathway is activated in response to intrinsic and extrinsic stresses, such as viral infections, hypoxia, glucose and amino acid deprivation, oncogene activation, UV radiation, and endoplasmic reticulum stress. Upon activation of ISR by one or more of these factors, the eukaryotic initiation factor 2 (eIF2, which is comprised of three subunits, α, β and γ) becomes phosphorylated in its α-subunit and rapidly reduces overall protein translation by binding to the eIF2B complex. This phosphorylation inhibits the eIF2B-mediated exchange of GDP for GTP (i.e., a guanine nucleotide exchange factor (GEF) activity), sequestering eIF2B in a complex with eIF2 and reducing general protein translation of most mRNA in the cell. Paradoxically, eIF2α phosphorylation also increases translation of a subset of mRNAs that contain one or more upstream open reading frames (uORFs) in their 5′ untranslated region (UTR). These transcripts include the transcriptional modulator activating transcription factor 4 (ATF4), transcriptional modulator activating transcription factor 3 (ATF3), the transcription factor CHOP, the growth arrest and DNA damage-inducible protein GADD34 and the β-secretase BACE-1.

Compounds useful in modulating the ISR pathway may be useful in treating a large number of diseases. In animals, the ISR pathway modulates a broad translational and transcriptional program involved in diverse processes such as learning memory, immunity, intermediary metabolism, insulin production and resistance to unfolded protein stress in the endoplasmic reticulum, among others. Activation of the ISR pathway has also been associated with numerous pathological conditions including cancer, neurodegenerative diseases, metabolic diseases (metabolic syndrome), autoimmune diseases, inflammatory diseases, musculoskeletal diseases (such as myopathy and muscle atrophy), vascular diseases, ocular diseases, and genetic disorders. Aberrant protein synthesis through eIF2α phosphorylation is also characteristic of several other human genetic disorders, cystic fibrosis, amyotrophic lateral sclerosis, Huntington disease and prion disease.

In addition, protein expression systems, such as cell-free protein expression systems or cell-based protein expression systems (i.e. eukaryotic cells, such as HEK cells, CHO cells, HeLa cells, myeloma cells, hybridoma cells, human blood-derived leukocytes, yeasts cells, wheat germ cells, insect cells, rabbit reticulocytes, or plant cells) allowing for the production of endogenous proteins have become important tools for a large number of businesses. These systems can be modified to express an increased amount of essential amino acids, to achieve greater yields of proteins express therein, or to produce recombinant proteins such as biopolymers, industrial proteins/enzymes, and therapeutic proteins.

In one particular application, genetically modifying plants to express heterologous proteins or increase the expression of endogenous proteins has become an important tool for a large number of business. Plants can be modified to express an increased amount of essential amino acids, to achieve greater yields of the plants or the proteins express therein, or to produce recombinant proteins such as biopolymers, industrial proteins/enzymes, and therapeutic proteins. However, there is a need to further increase the expression of plant proteins, which may require methods other than genetic modification. In addition, given the resistance to genetically modifying plants by some people, it may be desirable to increase protein production in plants using other methods. Increased protein production by plants will likely be essential for ensuring the availability of enough protein to feed an increasing world population under changing environmental conditions. Further, increased protein production in plants promote plant growth, because additional proteins can be released through the roots into the surrounding area to attract microorganisms, such as bacteria that can in turn improve plant development. One potential method of increasing protein production in plants is by modulating Integrated Stress Response (ISR) pathway.

### BRIEF SUMMARY

Modulators of the Integrated Stress Response (ISR) pathway are described, as are methods of making and using the compounds, or salts thereof.

In one aspect, provided is a compound of formula (I)

A-L¹-L²-L³-B-D-L⁴-E (I)

or a pharmaceutically acceptable salt thereof,
wherein:
A is selected from the group consisting of:
   C₁₀-C₁₄ aryl optionally substituted with 1, 2, 3, 4, 5, 6, 7, 8, or 9 R^{A} substituents;
   9-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)-$^{L2}, #^{A}—(C₁-C₆ alkylene)—O—$^{L2}, #^{A}—N(R^{L1})—$^{L2}, #^{A}—N(R^{L1})—(C₁-C₆ alkylene)-$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of:

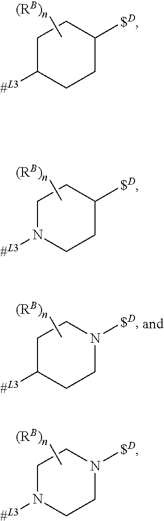

wherein
#^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
   C₄-C₁₄ cycloalkyl optionally substituted with 1, 2, 3, 4, 5, 6, 7, 8, or 9 R^{E} substituents;
   3-14 membered heterocycloalkyl optionally substituted with 1, 2, 3, 4, 5, 6, 7, 8, or 9 R^{E} substituents;
   C₆-C₁₄ aryl substituted with 1, 2, 3, 4, 5, 6, 7, 8, or 9 R^{E} substituents;
   5-14 membered heteroaryl substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)-NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)-NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)-NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)-NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
n is an integer selected from the group consisting of 0, 1, 2, 3, 4, 5, 6, 7, 8, and 9.

Provided in other aspects are compounds of Formula (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XIV-a), (XIV-a-i), (XV), (XV-i), (XVI), (XVII), (XVII-i), (XVII-a), (XVII-a-i), (XVIII), (XVIII-i), (XIX), (XIX-i), (XX), and (XX-i):

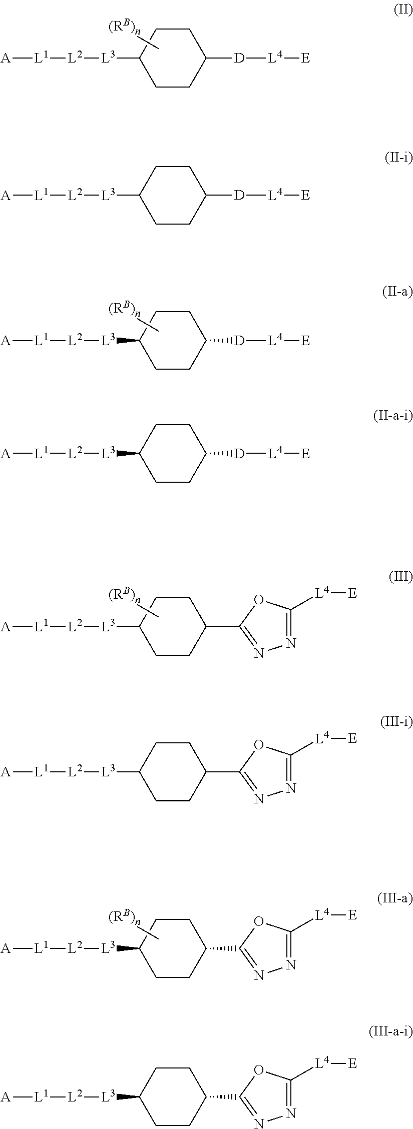

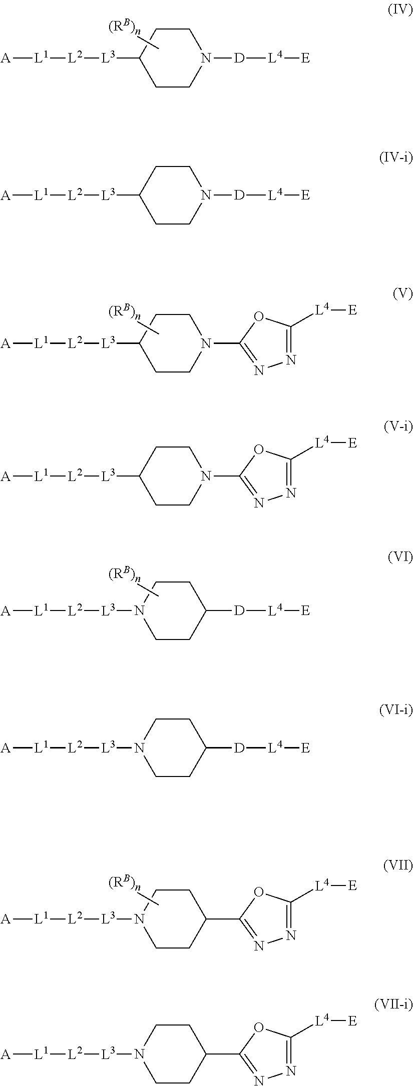

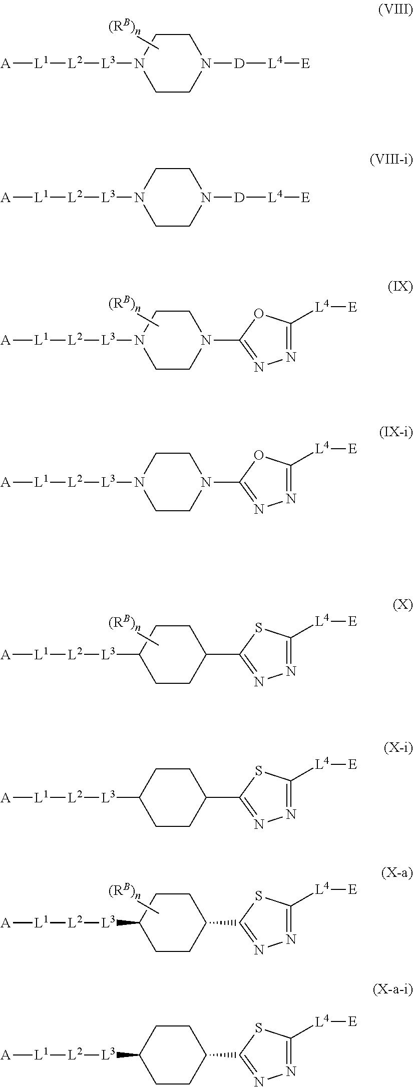

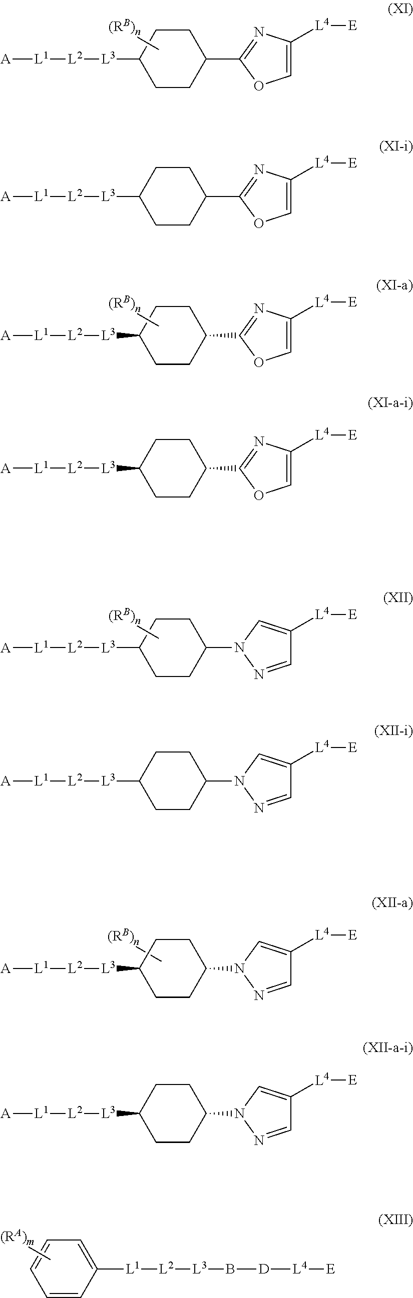

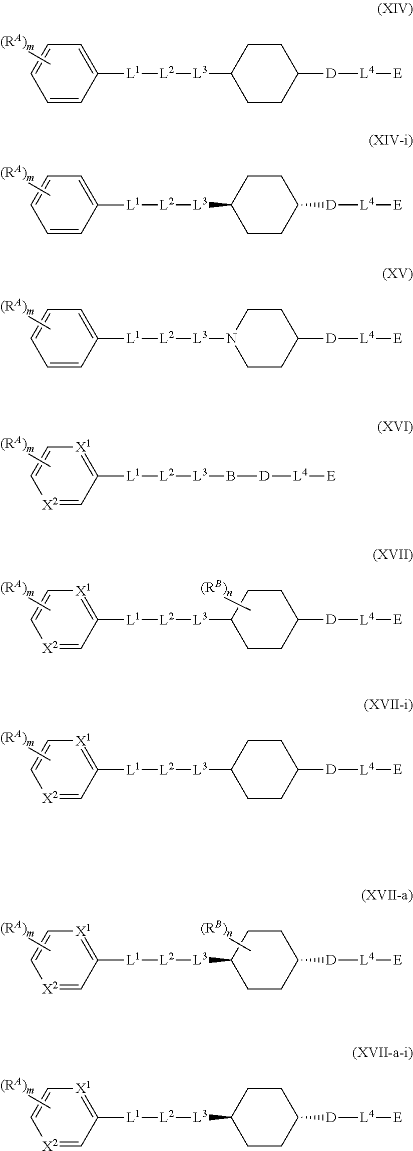

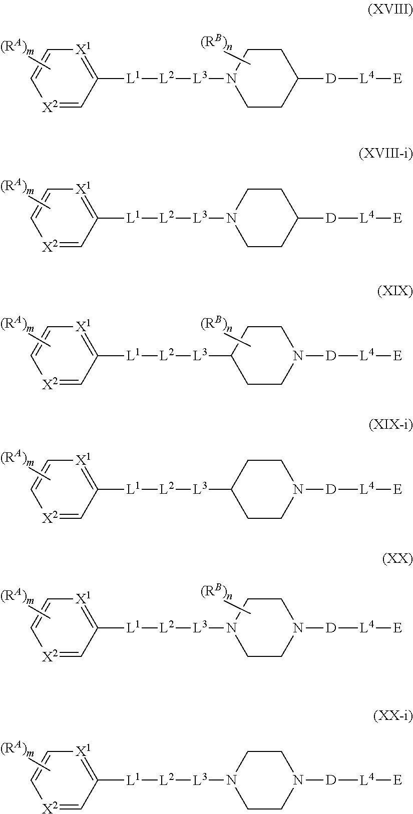

and pharmaceutically acceptable salts thereof;
wherein A, L¹, L², L³, L⁴, D, E, R^{A}, R^{B}, m, n, X¹, and X² are as defined hereinafter for the compounds of Formula (I), Formula (XIII), or Formula (XVI).

Provided in some embodiments are compounds of Table 1, Table 2, or Table 3, and pharmaceutically acceptable salts thereof.

In some aspects, provided are pharmaceutical compositions comprising a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a pharmaceutically acceptable salt thereof, and a pharmaceutically acceptable carrier.

Provided in some aspects are methods for enhancing protein synthesis in a living organism, comprising administering to the living organism an effective amount of a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof.

Provided in some aspects are methods for enhancing protein synthesis in a living organism, comprising administering to the living organism an effective amount of a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof.

Provided in some aspects are methods for accelerating growth of a plant, comprising administering to the plant an effective amount of a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof.

Provided in some aspects are methods for improving protein yield or quality in a plant, comprising administering to the plant an effective amount of a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof.

Provided in some aspects are methods of treating a disease or disorder mediated by an integrated stress response (ISR) pathway in an individual in need thereof comprising administering to the individual a therapeutically effective amount of a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a pharmaceutically acceptable salt thereof, or a therapeutically effective amount of a pharmaceutical composition as provided herein.

Provided in some aspects are methods of producing a protein, comprising contacting a eukaryotic cell comprising a nucleic acid encoding the protein with a compound of Formula (I), Formula (XIII), or Formula (XVI), such as a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof.

Provided in some aspects are methods method of culturing a eukaryotic cell comprising a nucleic acid encoding a protein, comprising contacting the eukaryotic cell with an in vitro culture medium comprising a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof

Provided in some aspects are methods of producing a protein, comprising contacting a cell-free protein synthesis (CFPS) system comprising eukaryotic initiation factor 2 (eIF2) and a nucleic acid encoding a protein with a compound Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof

Provided in some aspects are An in vitro cell culture medium, comprising a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof

Provided in some aspects are A cell-free protein synthesis (CFPS) system comprising eukaryotic initiation factor 2 (eIF2) and a nucleic acid encoding a protein with a compound of Formula (I), Formula (XIII), or Formula (XVI), such as a compound of formula (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), or (XX-i), or a compound of Table 1, Table 2, or Table 3, or a salt thereof.

### DESCRIPTION OF THE DRAWINGS

FIG. 1 shows relative fluorescence intensity (RFU) of GFP expressed in a Cell-free system in the presence of either vehicle or tested compounds.

### DETAILED DESCRIPTION

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). An alkenyl group may have “cis” or “trans” configurations, or alternatively have “E” or “Z” configurations. Particular alkenyl groups are those having 2 to 20 carbon atoms (a “C₂-C₂₀ alkenyl”), having 6 to 10 carbon atoms (a “C₆-C₁₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl group include, but are not limited to, groups such as ethenyl (or vinyl), prop-i-enyl, prop-2-enyl (or allyl), 2-methylprop-1-enyl, but-i-enyl, but-2-enyl, but-3-enyl, buta-1,3-dienyl, 2-methylbuta-1,3-dienyl, pent-i-enyl, pent-2-enyl, hex-i-enyl, hex-2-enyl, hex-3-enyl, and the like.

“Alkenylene” as used herein refers to the same residues as alkenyl, but having bivalency. Particular alkenylene groups are those having 2 to 20 carbon atoms (a “C₂-C₂₀ alkenylene”), having 2 to 10 carbon atoms (a “C₂-C₁₀ alkenylene”), having 6 to 10 carbon atoms (a “C₆-C₁₀ alkenylene”), having 2 to 6 carbon atoms (a “C₂-C₆ alkenylene”), 2 to 4 carbon atoms (a “C₂-C₄ alkenylene”) or 2 to 3 carbon atoms (a “C₂-C₃ alkenylene”). Examples of alkenylene include, but are not limited to, groups such as ethenylene (or vinylene) (—CH═CH—), propenylene (—CH═CHCH₂—), 1,4-but-1-enylene (—CH═CH—CH₂CH₂—), 1,4-but-2-enylene (—CH₂CH═CHCH₂—), 1,6-hex-i-enylene (—CH═CH—(CH₂)₃CH₂—), and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 6 to 10 carbon atoms (a “C₆-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-i-ynyl, prop-2-ynyl (or propargyl), but-i-ynyl, but-2-ynyl, but-3-ynyl, and the like.

“Alkynylene” as used herein refers to the same residues as alkynyl, but having bivalency. Particular alkynylene groups are those having 2 to 20 carbon atoms (a “C₂-C₂₀ alkynylene”), having 2 to 10 carbon atoms (a “C₂-C₁₀ alkynylene”), having 6 to 10 carbon atoms (a “C₆-C₁₀ alkynylene”), having 2 to 6 carbon atoms (a “C₂-C₆ alkynylene”), 2 to 4 carbon atoms (a “C₂-C₄ alkynylene”) or 2 to 3 carbon atoms (a “C₂-C₃ alkynylene”). Examples of alkynylene include, but are not limited to, groups such as ethynylene (or acetylenylene) (—C≡C—), propynylene (—C≡CCH₂—), and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 14 annular carbon atoms. A preferred cycloalkyl is a cyclic hydrocarbon having from 3 to 12 annular carbon atoms (a “C₃-C₁₂ cycloalkyl”), 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Cycloalkylene” as used herein refers to the same residues as cycloalkyl, but having bivalency. Cycloalkylene can consist of one ring or multiple rings which may be fused, spiro or bridged, or combinations thereof. Particular cycloalkylene groups are those having from 3 to 14 annular carbon atoms. A preferred cycloalkylene is a cyclic hydrocarbon having from 3 to 12 annular carbon atoms (a “C₃-C₁₂ cycloalkylene”), having from 3 to 8 annular carbon atoms (a “C₃-C₈ cycloalkylene”), having 3 to 6 carbon atoms (a “C₃-C₆ cycloalkylene”), or having from 3 to 4 annular carbon atoms (a “C₃-C₄ cycloalkylene”). Examples of cycloalkylene include, but are not limited to, cyclopropylene, cyclobutylene, cyclopentylene, cyclohexylene, cycloheptylene, norbornylene, and the like. A cycloalkylene may attach to the remaining structures via the same ring carbon atom or different ring carbon atoms. When a cycloalkylene attaches to the remaining structures via two different ring carbon atoms, the connecting bonds may be cis- or trans- to each other. For example, cyclopropylene may include 1,1-cyclopropylene and 1,2-cyclopropylene (e.g., cis-1,2-cyclopropylene or trans-1,2-cyclopropylene), or a mixture thereof.

“Cycloalkenyl” refers to and includes, unless otherwise stated, an unsaturated cyclic non-aromatic univalent hydrocarbon structure, having at least one site of olefinic unsaturation (i.e., having at least one moiety of the formula C═C) and having the number of carbon atoms designated (i.e., C₂-C₁₀ means two to ten carbon atoms). Cycloalkenyl can consist of one ring, such as cyclohexenyl, or multiple rings, such as norbornenyl. A preferred cycloalkenyl is an unsaturated cyclic hydrocarbon having from 3 to 8 annular carbon atoms (a “C₃-C₈ cycloalkenyl”). Examples of cycloalkenyl groups include, but are not limited to, cyclopropenyl, cyclobutenyl, cyclopentenyl, cyclohexenyl, norbornenyl, and the like.

“Cycloalkenylene” as used herein refers to the same residues as cycloalkenyl, but having bivalency.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic is connected to the parent structure at an aromatic ring position.

“Arylene” as used herein refers to the same residues as aryl, but having bivalency. Particular arylene groups are those having from 6 to 14 annular carbon atoms (a “C₆-C₁₄ arylene”).

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group having more than one ring where at least one ring is non-aromatic is connected to the parent structure at an aromatic ring position. A heteroaryl group may be connected to the parent structure at a ring carbon atom or a ring heteroatom.

“Heteroarylene” as used herein refers to the same residues as heteroaryl, but having bivalency.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Heterocyclylene” as used herein refers to the same residues as heterocyclyl, but having bivalency.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

As used herein, the term “agriculturally effective amount” refers to an amount of a compound or salt thereof sufficient to produce a desired agricultural outcome in a plant. Accordingly, in some embodiments, an agriculturally effective amount may increase protein expression, increase growth, and/or alter the microbial environment adjacent to the plant.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “agriculturally acceptable salt” refers to a salt which retains at least some of the biological activity of the free (non-salt) compound and which can be administered to plants. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Agriculturally acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

It is understood that aspects and embodiments described herein as “comprising” include “consisting of” and “consisting essentially of” embodiments.

When a composition is described as “consisting essentially of” the listed components, the composition contains the components expressly listed, and may contain other components which do not substantially affect the disease or condition being treated such as trace impurities. However, the composition either does not contain any other components which do substantially affect the disease or condition being treated other than those components expressly listed; or, if the composition does contain extra components other than those listed which substantially affect the disease or condition being treated, the composition does not contain a sufficient concentration or amount of those extra components to substantially affect the disease or condition being treated. When a method is described as “consisting essentially of” the listed steps, the method contains the steps listed, and may contain other steps that do not substantially affect the disease or condition being treated, but the method does not contain any other steps which substantially affect the disease or condition being treated other than those steps expressly listed.

When a moiety is indicated as substituted by “at least one” substituent, this also encompasses the disclosure of exactly one substituent.

### Compounds

In a first aspect, provided is a compound of formula (I)

A-L¹-L²-L³-B-D-L⁴-E (I)

or a pharmaceutically acceptable salt thereof,
wherein:
A is selected from the group consisting of:
   C₁₀-C₁₄ aryl optionally substituted with 1, 2, 3, 4, 5, 6, 7, 8, or 9 R^{A} substituents;
   9-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of:

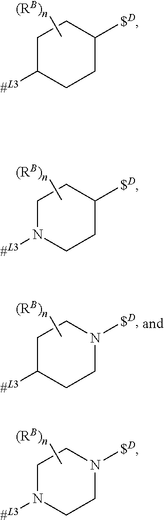

wherein
#^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
   C₃-C₁₄ cycloalkyl optionally substituted with 1, 2, 3, 4, 5, 6, 7, 8, or 9 R^{E} substituents;
   3-14 membered heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)-NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)-NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)-NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)-NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
   n is an integer selected from the group consisting of 0, 1, 2, 3, 4, 5, 6, 7, 8, and 9.

In some embodiments, A is selected from the group consisting of:
C₆-C₁₄ aryl optionally substituted with 1, 2, 3, 4, 5, 6, 7, 8, or 9 R^{A} substituents;
5-14 membered heteroaryl optionally substituted with 1, 2, 3, 4, 5, 6, 7, 8, or 9 R^{A} substituents; and
8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of C₃-C₁₄ cycloalkyl, C₃-C₁₄ cycloalkenyl, 3-14 membered heterocycloalkyl, 3-14 membered heterocycloalkenyl, C₆-C₁₄ aryl, and 5-14 membered heteroaryl, wherein each of the C₃-C₁₄ cycloalkyl, C₃-C₁₄ cycloalkenyl, 3-14 membered heterocycloalkyl, 3-14 membered heterocycloalkenyl, C₆-C₁₄ aryl, and 5-14 membered heteroaryl, are optionally substituted with 1, 2, 3, 4, 5, 6, 7, 8, or 9 R^{B} substituents;
D is a 5-membered carbocycle, 5-membered heterocycle, or 5-membered heteroaryl, each optionally substituted with 1, 2, 3, 4, 5, 6, 7, or 8 R^{D} substituents;
L⁴ is a bond or #^{D}—CH₂—O—$^{E}, wherein #^{D} represents the attachment point to D and $^{E} represents the attachment point to E;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)-NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)-NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)-NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)-NH—(C₁-C₆ haloalkyl);
R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments of the compounds of formula (I), or the salts thereof, B is selected from the group consisting of C₃-C₁₄ cycloalkyl, C₃-C₁₄ cycloalkenyl, 3-14 membered heterocycloalkyl, 3-14 membered heterocycloalkenyl, C₆-C₁₄ aryl, and 5-14 membered heteroaryl, wherein each of the C₃-C₁₄ cycloalkyl, C₃-C₁₄ cycloalkenyl, 3-14 membered heterocycloalkyl, 3-14 membered heterocycloalkenyl, C₆-C₁₄ aryl, and 5-14 membered heteroaryl, are optionally substituted with 1, 2, 3, 4, 5, 6, 7, 8, or 9 R^{B} substituents. In some embodiments, B is selected from the group consisting of C₃-C₁₄ cycloalkyl, C₃-C₁₄ cycloalkenyl, 3-14 membered heterocycloalkyl, and 3-14 membered heterocycloalkenyl, wherein each of the C₃-C₁₄ cycloalkyl, C₃-C₁₄ cycloalkenyl, 3-14 membered heterocycloalkyl, and 3-14 membered heterocycloalkenyl are optionally substituted with 1, 2, 3, 4, 5, 6, 7, 8, or 9 R^{B} substituents. In some embodiments, B is selected from the group consisting of C₃-C₁₄ cycloalkyl and 3-14 membered heterocycloalkyl, wherein each of the C₃-C₁₄ cycloalkyl and 3-14 membered heterocycloalkenyl are optionally substituted with 1, 2, 3, 4, 5, 6, 7, 8, or 9 R^{B} substituents. In some embodiments, B is C₃-C₁₄ cycloalkyl, optionally substituted with 1, 2, 3, 4, 5, 6, 7, 8, or 9 R^{B} substituents, and wherein the C₃-C₁₄ cycloalkyl is selected from the group consisting of monocyclic C₃-C₁₄ cycloalkyl and polycyclic C₃-C₁₄ cycloalkyl, wherein each of the monocyclic C₃-C₁₄ cycloalkyl and polycyclic C₃-C₁₄ cycloalkyl are optionally substituted with 1, 2, 3, 4, 5, 6, 7, 8, or 9 R^{B} substituents. In some embodiments, B is polycyclic C₃-C₁₄ cycloalkyl, optionally substituted with 1, 2, 3, 4, 5, 6, 7, 8, or 9 R^{B} substituents, and wherein the polycyclic C₃-C₁₄ cycloalkyl is selected from the group consisting of fused polycyclic C₃-C₁₄ cycloalkyl, bridged polycyclic C₃-C₁₄ cycloalkyl, and spiro polycyclic C₃-C₁₄ cycloalkyl, wherein each of the fused polycyclic C₃-C₁₄ cycloalkyl, bridged polycyclic C₃-C₁₄ cycloalkyl, and spiro polycyclic C₃-C₁₄ cycloalkyl are optionally substituted with 1, 2, 3, 4, 5, 6, 7, 8, or 9 R^{B} substituents. In some embodiments, B is 3-14 membered heterocycloalkyl, optionally substituted with 1, 2, 3, 4, 5, 6, 7, 8, or 9 R^{B} substituents, and wherein the 3-14 membered heterocycloalkyl is selected from the group consisting of monocyclic 3-14 membered heterocycloalkyl and polycyclic 3-14 membered heterocycloalkyl, wherein each of the monocyclic C₃-C₁₄ cycloalkyl and polycyclic C₃-C₁₄ cycloalkyl are optionally substituted with 1, 2, 3, 4, 5, 6, 7, 8, or 9 R^{B} substituents. In some embodiments, B is polycyclic 3-14 membered heterocycloalkyl, optionally substituted with 1, 2, 3, 4, 5, 6, 7, 8, or 9 R^{B} substituents, and wherein the polycyclic 3-14 membered heterocycloalkyl is selected from the group consisting of fused polycyclic 3-14 membered heterocycloalkyl, bridged polycyclic 3-14 membered heterocycloalkyl, and spiro polycyclic 3-14 membered heterocycloalkyl, wherein each of the fused polycyclic 3-14 membered heterocycloalkyl, bridged polycyclic 3-14 membered heterocycloalkyl, and spiro polycyclic 3-14 membered heterocycloalkyl are optionally substituted with 1, 2, 3, 4, 5, 6, 7, 8, or 9 R^{B} substituents. In some embodiments, B is selected from the group consisting of:

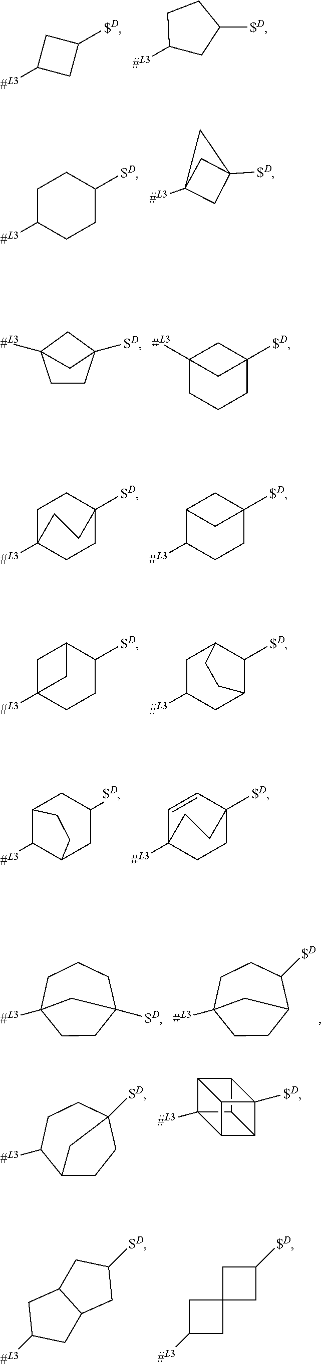

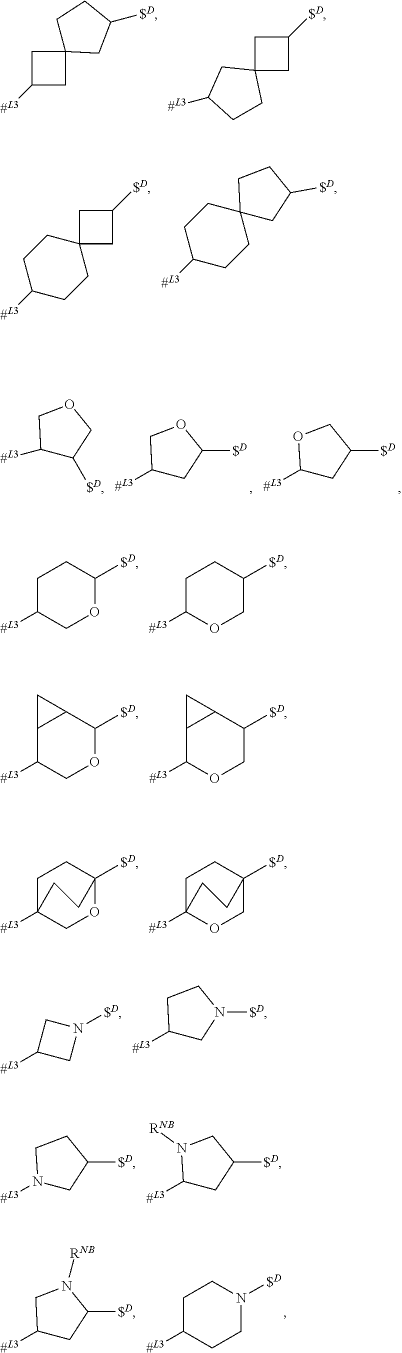

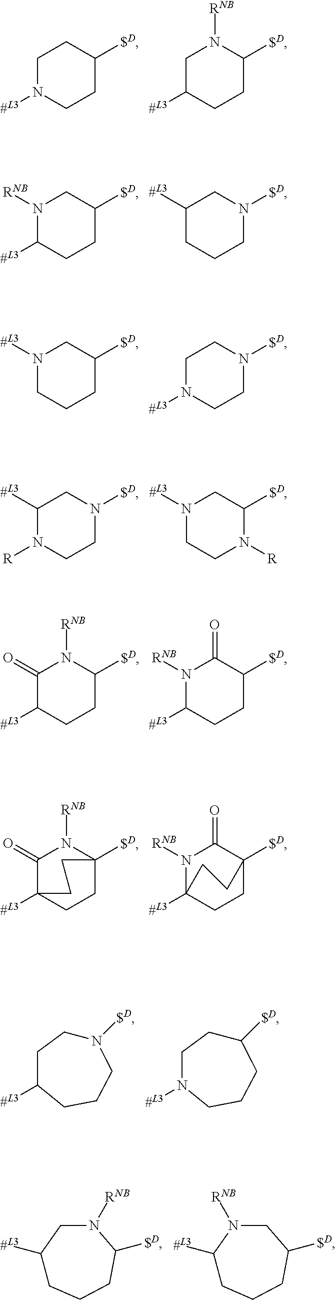

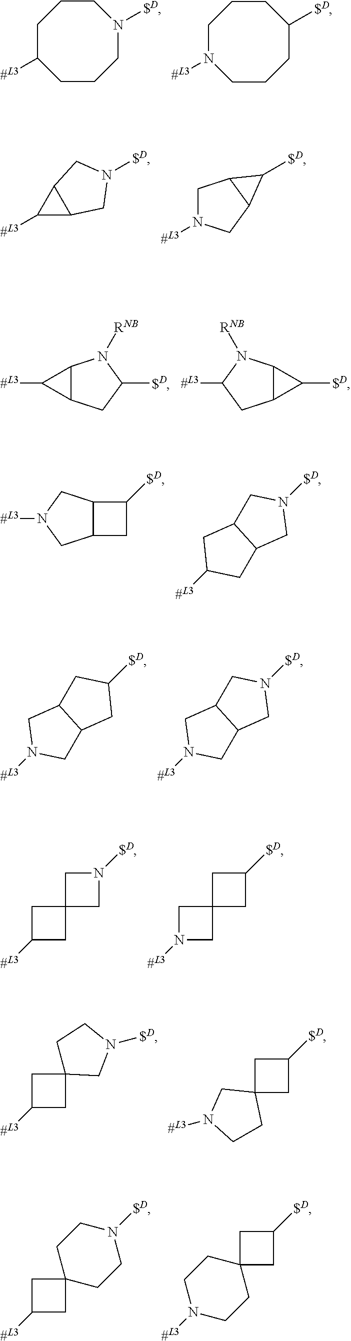

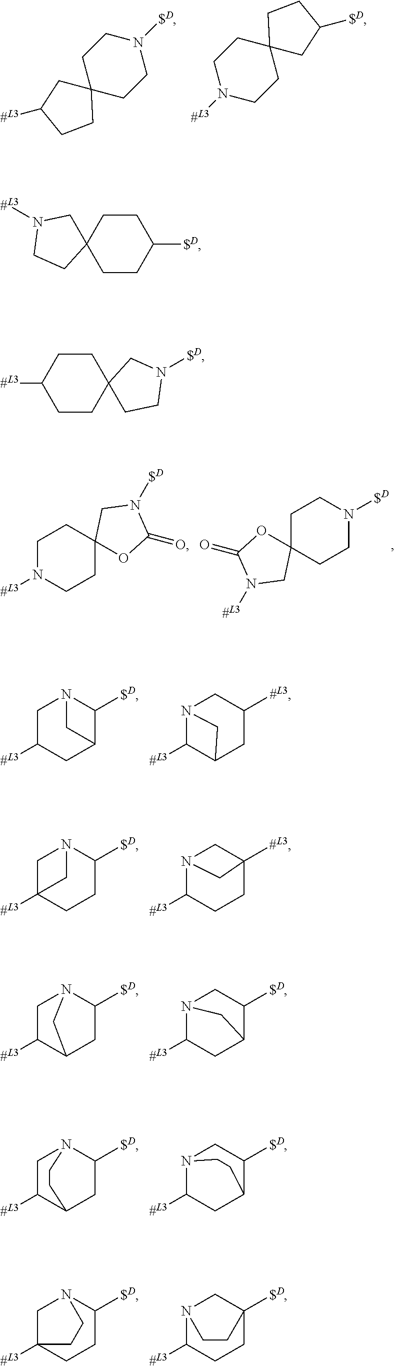

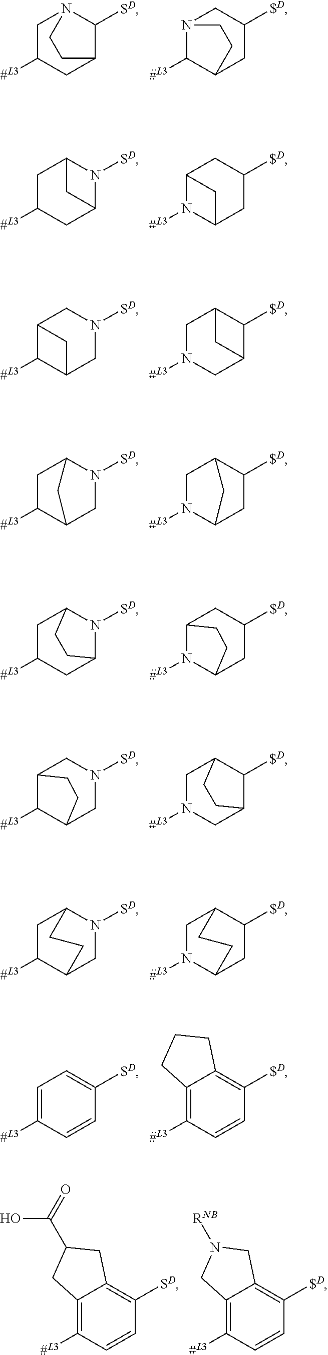

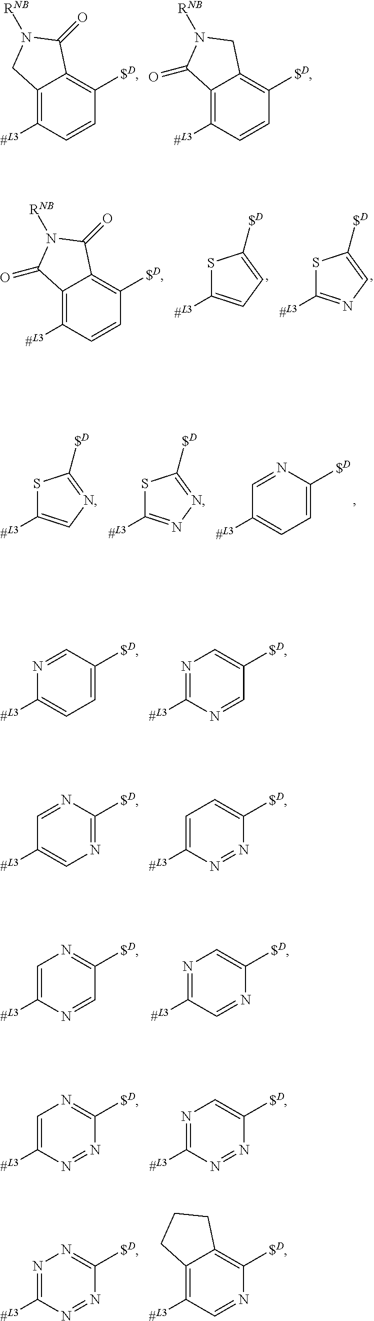

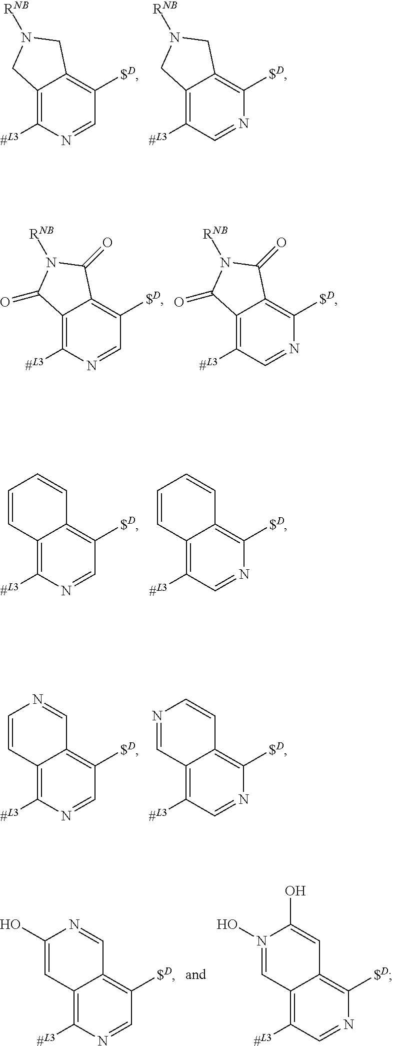

wherein each of the foregoing is optionally substituted with 1, 2, 3, 4, 5, 6, 7, 8, or 9 R^{BB} substituents;
wherein R^{NB} is selected from the group consisting of hydrogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents; and wherein #^{L3} represents the attachment point to L³ and $^{D} represents the attachment point to D. In some embodiments, B is selected from the group consisting of:

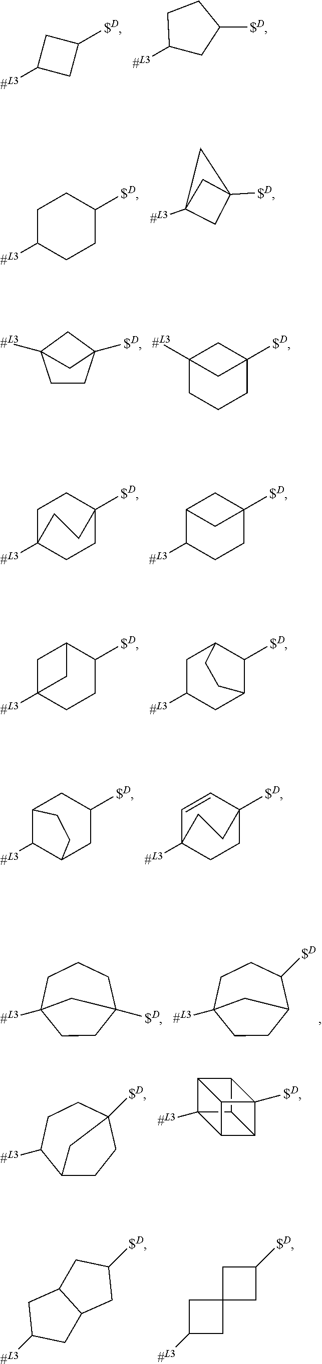

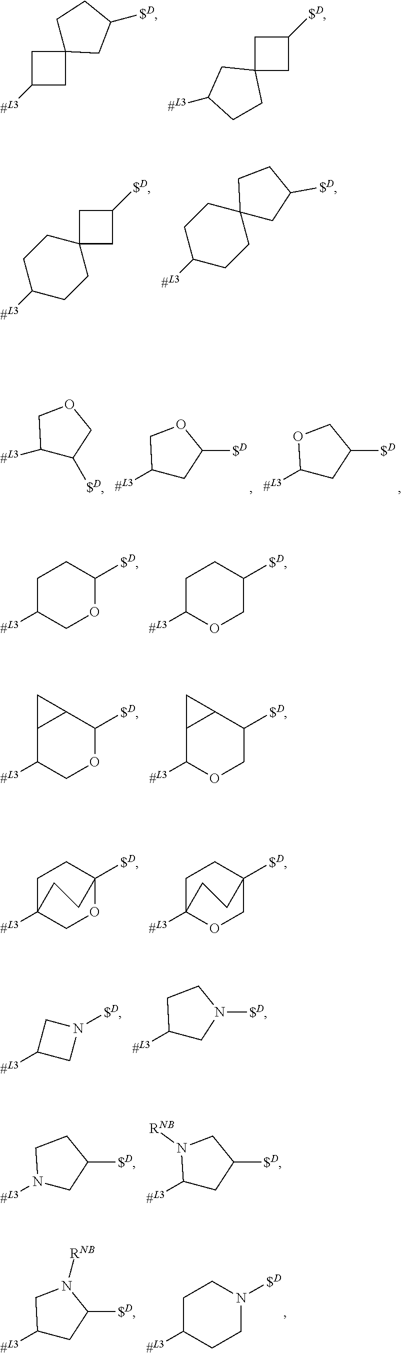

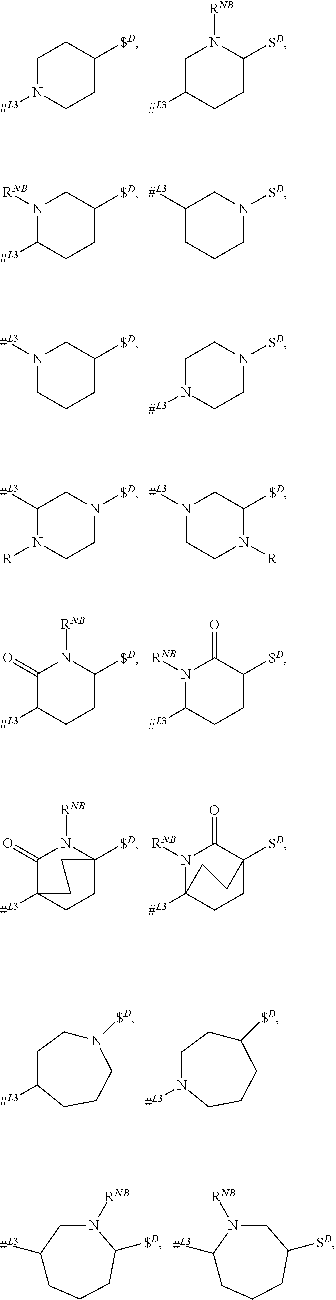

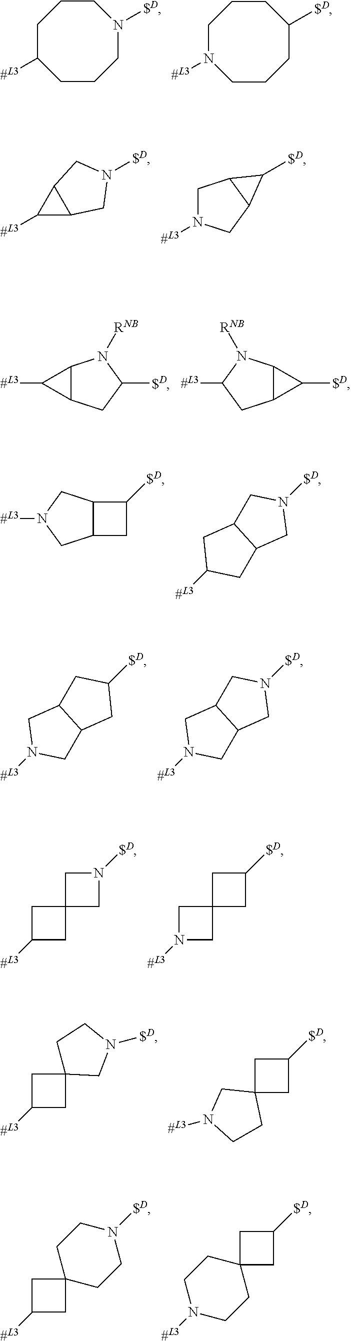

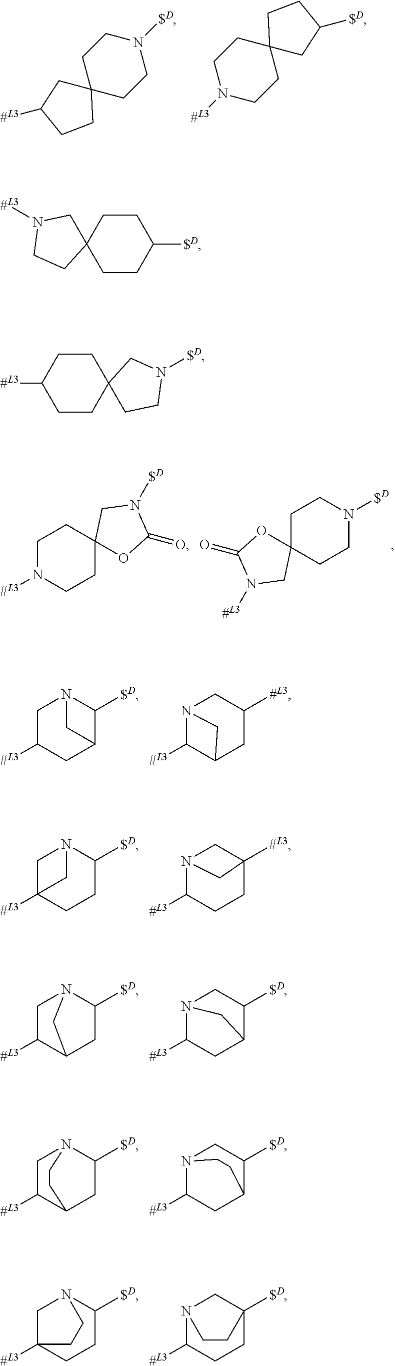

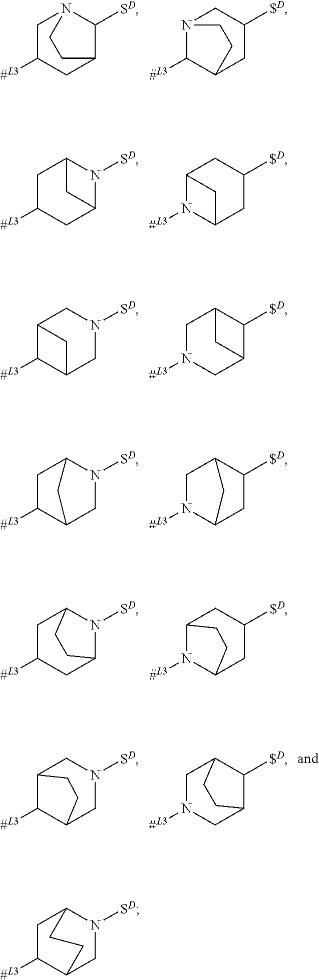

wherein each of the foregoing is optionally substituted with 1, 2, 3, 4, 5, 6, 7, 8, or 9 R^{BB} substituents;
wherein R^{NB} is selected from the group consisting of hydrogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents; and wherein #^{L3} represents the attachment point to L³ and $^{D} represents the attachment point to D. In some embodiments, B is selected from the group consisting of:

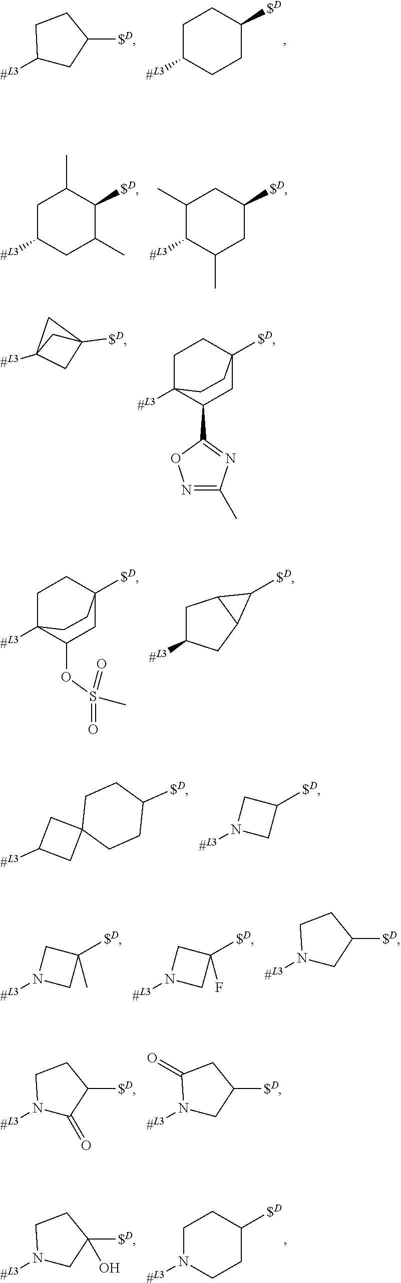

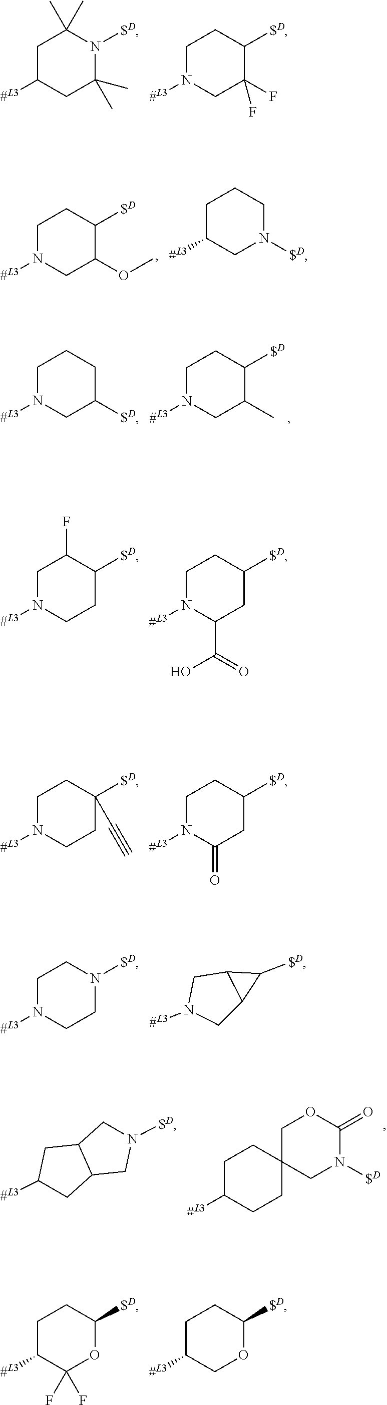

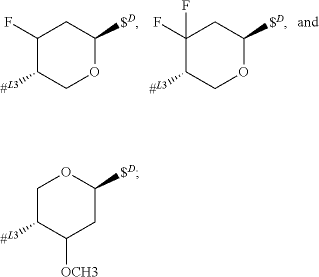

wherein each of the foregoing is optionally substituted with 1, 2, 3, 4, 5, 6, 7, 8, or 9 R^{BB} substituents; and
wherein #^{L3} represents the attachment point to L³ and $^{D} represents the attachment point to D. In some embodiments, B is selected from the group consisting of:

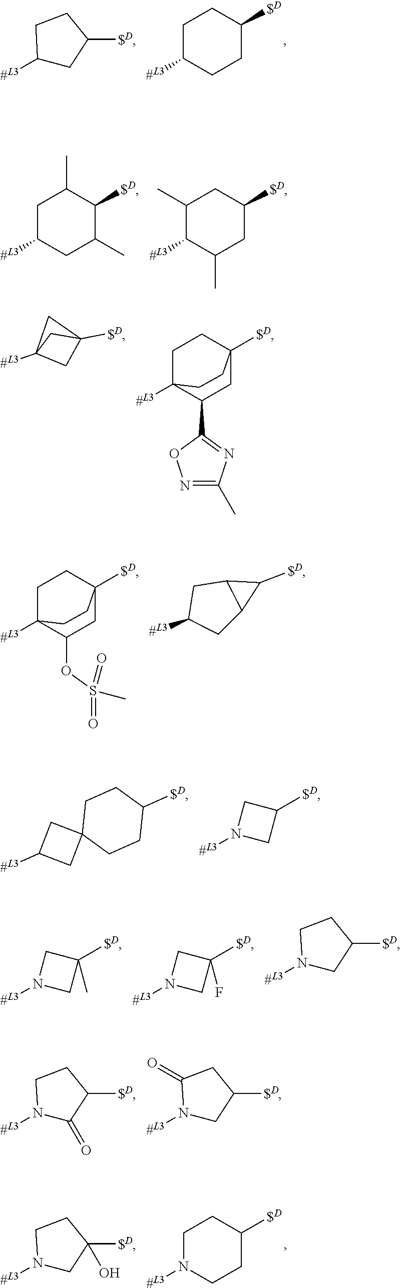

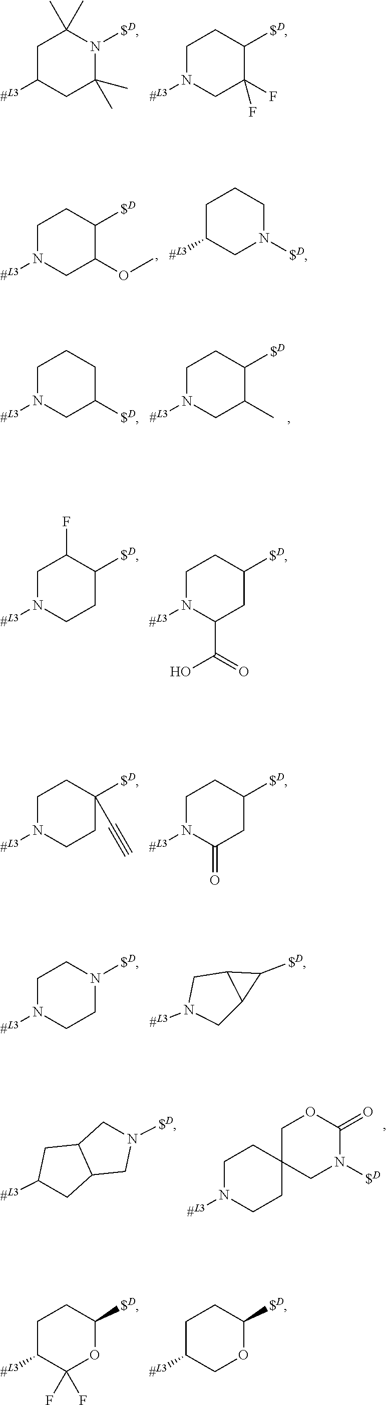

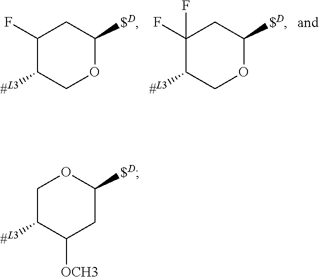

wherein #^{L3} represents the attachment point to L³ and $^{D} represents the attachment point to D. In some embodiments, R^{B}, independently at each occurrence, is selected from the group consisting of halogen, oxo, C₁-C₆ alkyl, C₂-C₆ alkynyl, OH, O(C₁-C₆ alkyl), C(O)OH, OS(O)₂(C₁-C₆ alkyl), and 5-14 membered heteroaryl optionally substituted with 1, 2, 3, 4, 5, 6, 7, 8, or 9 R^{BB} substituents. In some embodiments, R^{B}, independently at each occurrence, is selected from the group consisting of fluoro, oxo, methyl, ethynyl, OH, OCH₃, C(O)OH, OS(O)₂CH₃, and 3-methyl-1,2,4-oxadiazol-5-yl. In some embodiments, B is selected from the group consisting of cyclohexyl and 6 membered heterocycloalkyl, wherein each of the cyclohexyl and 6 membered heterocycloalkyl are optionally substituted with 1, 2, 3, 4, 5, 6, 7, 8, or 9 R^{B} substituents. In some embodiments, B is cyclohexyl, optionally substituted with 1, 2, 3, 4, 5, 6, 7, 8, or 9 R^{B} substituents. In some embodiments, B is 6 membered heterocycloalkyl, optionally substituted with 1, 2, 3, 4, 5, 6, 7, 8, or 9 R^{B} substituents. In some embodiments, B is selected from the group consisting of:

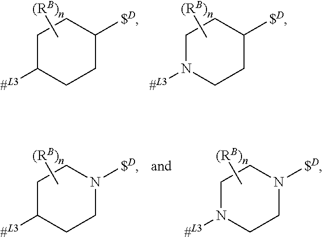

wherein #^{L3} represents the attachment point to L³ and $^{D} represents the attachment point to D. In some embodiments, B is

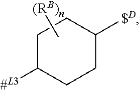

wherein #^{L3} represents the attachment point to L³ and $^{D} represents the attachment point to D. In some embodiments, B is

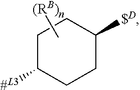

wherein #^{L3} represents the attachment point to L³ and $^{D} represents the attachment point to D. In some embodiments, B is

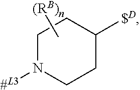

wherein #^{L3} represents the attachment point to L³ and $^{D} represents the attachment point to D. In some embodiments, B is

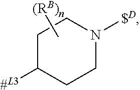

wherein #^{L3} represents the attachment point to L³ and $^{D} represents the attachment point to D. In some embodiments, B is

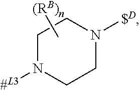

wherein #^{L3} represents the attachment point to L³ and $^{D} represents the attachment point to D. In some embodiments, B is selected from the group consisting of:

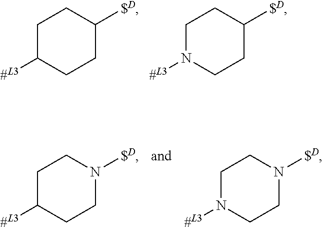

wherein #^{L3} represents the attachment point to L³ and $^{D} represents the attachment point to D. In some embodiments, B is

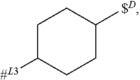

wherein #^{L3} represents the attachment point to L³ and $^{D} represents the attachment point to D. In some embodiments, B is

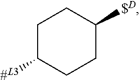

wherein #^{L3} represents the attachment point to L³ and $^{D} represents the attachment point to D. In some embodiments, B is

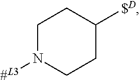

wherein #^{L3} represents the attachment point to L³ and $^{D} represents the attachment point to D. In some embodiments, B is

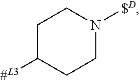

wherein #^{L3} represents the attachment point to L³ and $^{D} represents the attachment point to D. In some embodiments, B is

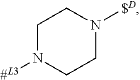

wherein #^{L3} represents the attachment point to L³ and $^{D} represents the attachment point to D.

In some embodiments of the compounds of formula (I), or the salts thereof, D is a 5-membered carbocycle, 5-membered heterocycle, or 5-membered heteroaryl, each optionally substituted with 1, 2, 3, 4, 5, 6, 7, or 8 R^{D} substituents. In some embodiments, D is selected from the group consisting of:

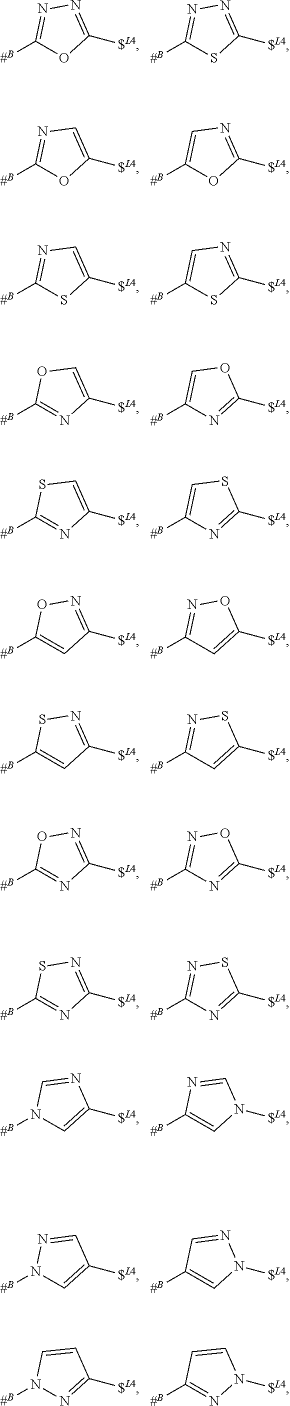

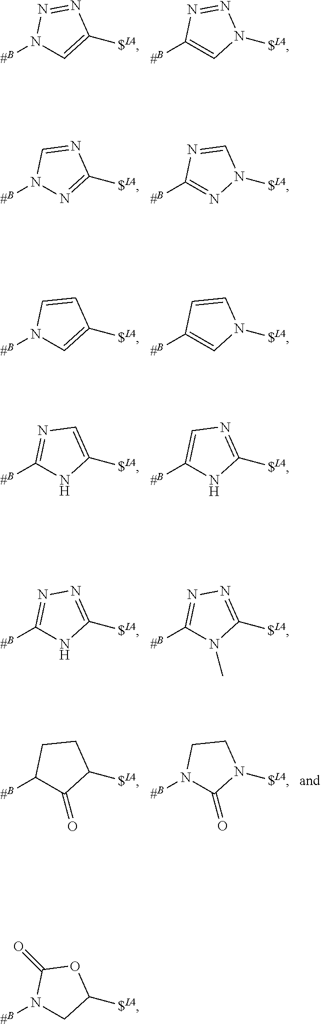

wherein #^{B} represents the attachment point to B and $^{L4} represents the attachment point to L⁴.
In some embodiments, D is selected from the group consisting of:

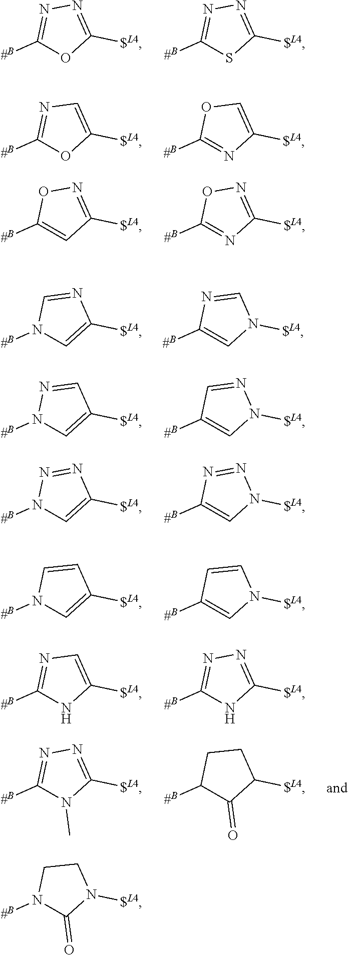

wherein #^{B} represents the attachment point to B and $^{L4} represents the attachment point to L⁴. In some embodiments, D is 5-membered heteroaryl optionally substituted with 1, 2, 3, 4, 5, 6, 7, or 8 RP substituents. In some embodiments, D is selected from the group consisting of:

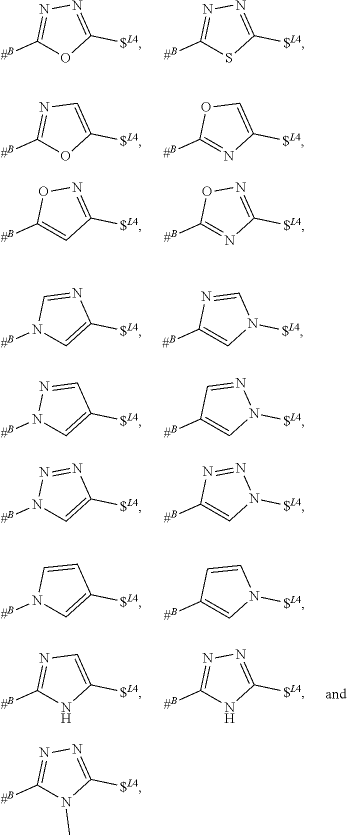

wherein #^{B} represents the attachment point to B and $^{L4} represents the attachment point to L⁴. In some embodiments, D is

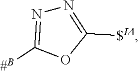

wherein #^{B} represents the attachment point to B and $^{L4} represents the attachment point to L4. In some embodiments, D is

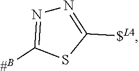

wherein #^{B} represents the attachment point to B and $^{L4} represents the attachment point to L⁴. In some embodiments, D is

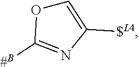

wherein #^{B} represents the attachment point to B and $^{L4} represents the attachment point to L4. In some embodiments, D is

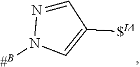

wherein #^{B} represents the attachment point to B and $^{L4} represents the attachment point to L⁴. In some embodiments, D is 5-membered carbocycle optionally substituted with 1, 2, 3, 4, 5, 6, 7, or 8 R^{D} substituents. In some embodiments, D is

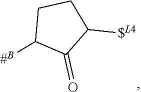

wherein #^{B} represents the attachment point to B and $^{L4} represents the attachment point to L⁴. In some embodiments, D is 5-membered heterocycle optionally substituted with 1, 2, 3, 4, 5, 6, 7, or 8 R^{D} substituents. In some embodiments, D is

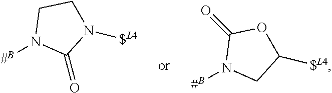

wherein #^{B} represents the attachment point to B and $^{L4} represents the attachment point to L⁴.

In some embodiments, A is selected from the group consisting of:
C₁₀-C₁₄ aryl optionally substituted with 1, 2, 3, 4, 5, 6, 7, 8, or 9 R^{A} substituents;
9-14 membered heteroaryl optionally substituted with 1, 2, 3, 4, 5, 6, 7, 8, or 9 R^{A} substituents; and
8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of:

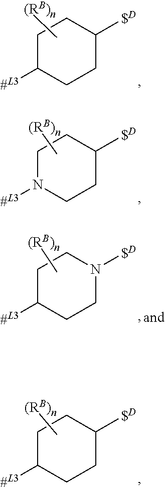

wherein
#^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
   C₄-C₁₄ cycloalkyl optionally substituted with 1, 2, 3, 4, 5, 6, 7, 8, or 9 R^{E} substituents;
   3-14 membered heterocycloalkyl optionally substituted with 1, 2, 3, 4, 5, 6, 7, 8, or 9 R^{E} substituents;
   C₆-C₁₄ aryl substituted with 1, 2, 3, 4, 5, 6, 7, 8, or 9 R^{E} substituents;
   5-14 membered heteroaryl substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)-NH—(C₁—C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)-NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)-NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)-NH—(C₁-C₆ haloalkyl);
R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
n is an integer selected from the group consisting of 0, 1, 2, 3, 4, 5, 6, 7, 8, and 9.

In some embodiments, the compound of formula (I), or the salt thereof, is a compound of is a compound of formula (II):

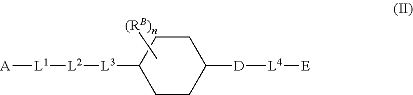

or a pharmaceutically acceptable salt thereof. In some embodiments, D is selected from the group consisting of:

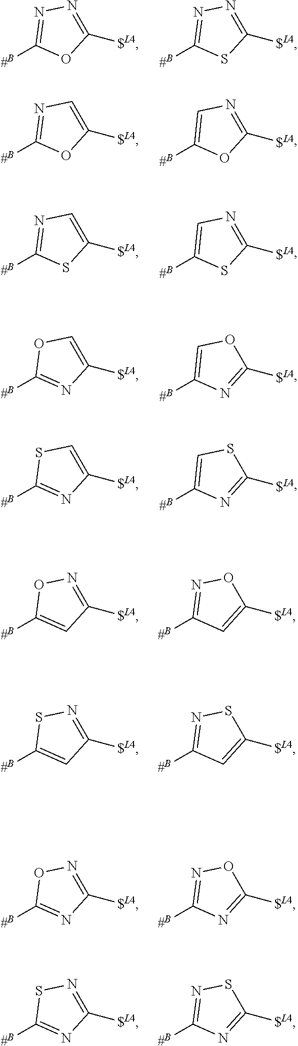

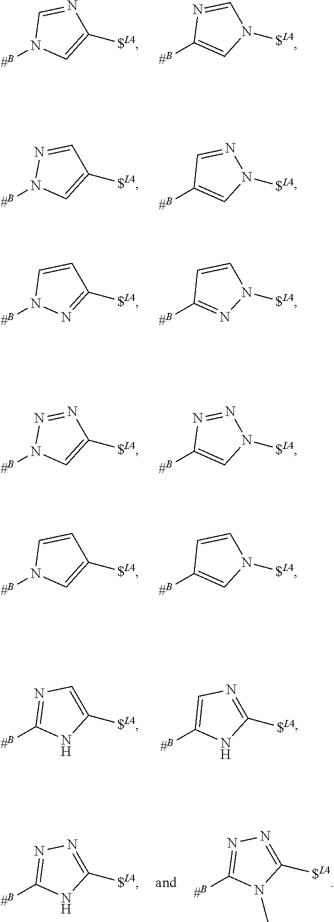

In some embodiments, D is selected from the group consisting of:

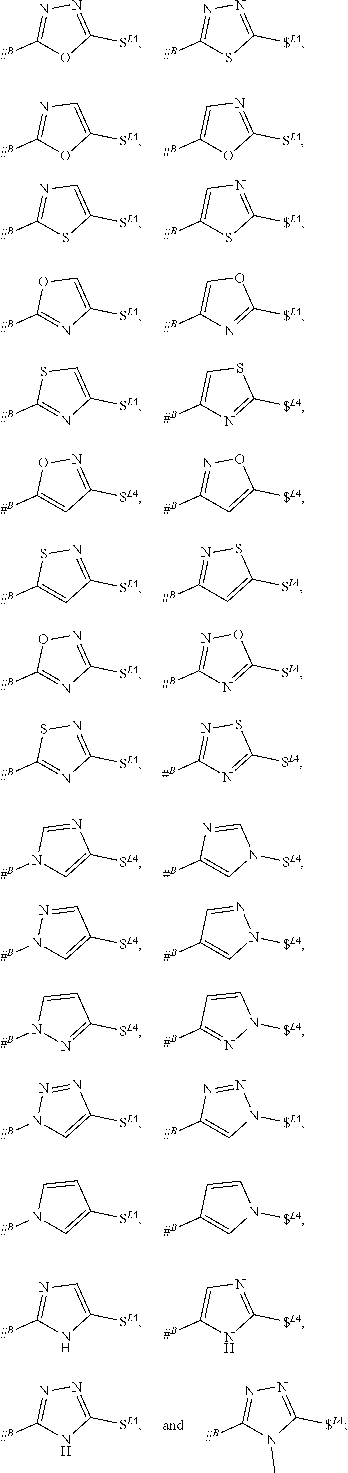

A is selected from the group consisting of:
   C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
   9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
   8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
   C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
   8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
n is an integer selected from the group consisting of 0, 1, 2, 3, 4, 5, 6, 7, 8, and 9, and
provided that when L² is #^{L1}—N(R^{L2})—C(O)—$^{L3}, then L¹ is selected from the group consisting of a bond, #^{A}—(C₁-C₆ alkylene)—$^{L2}, #^{A}—O—$^{L2}, #^{A}—O—(C₁-C₆ alkylene)—$^{L2}, and #^{A}—N(R^{L1})—$^{L2}.
   In some embodiments, D is selected from the group consisting of:

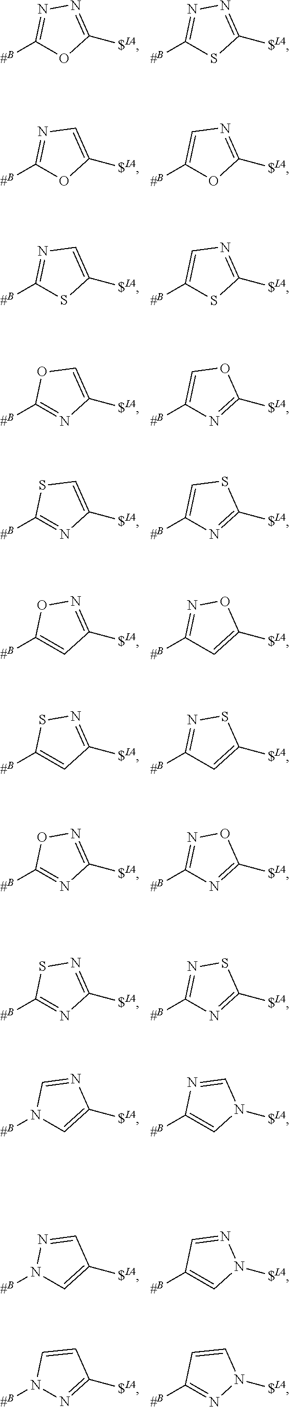

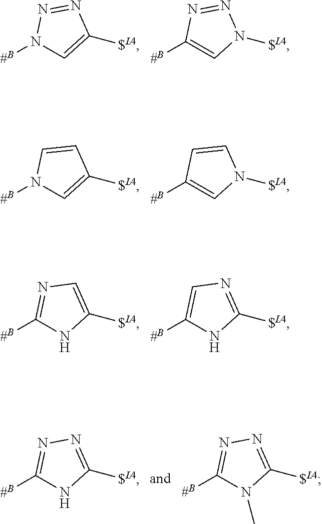

A is selected from the group consisting of:

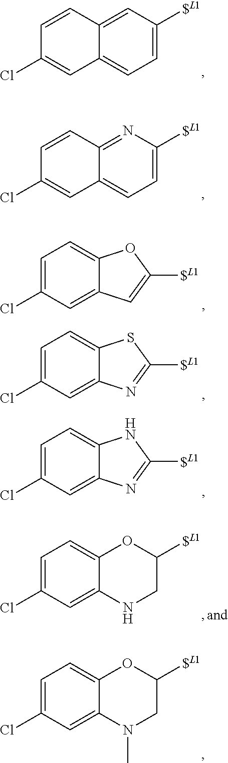

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (II), or the salt thereof, is a compound of is a compound of formula (II-i):

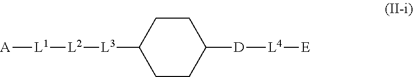

or a pharmaceutically acceptable salt thereof. In some embodiments, D is selected from the group consisting of:

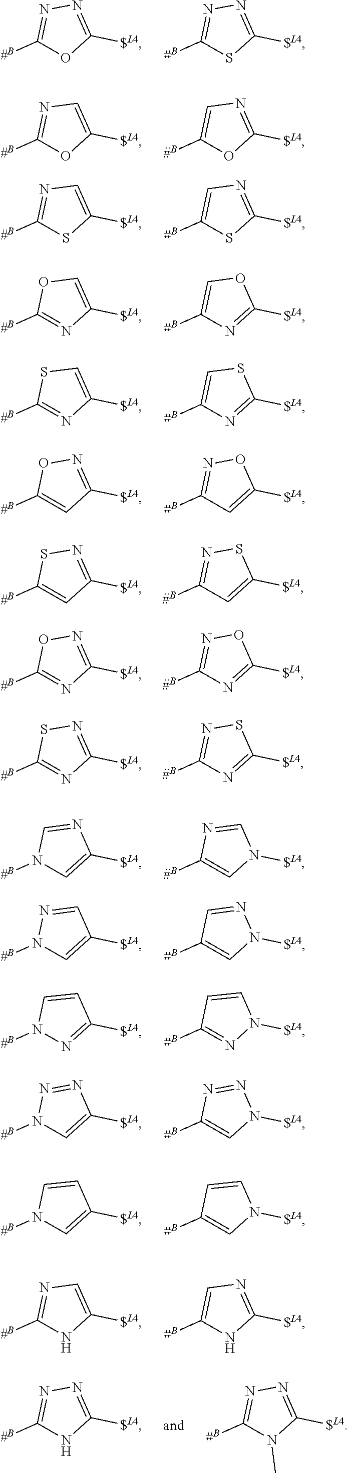

In some embodiments, D is selected from the group consisting of:

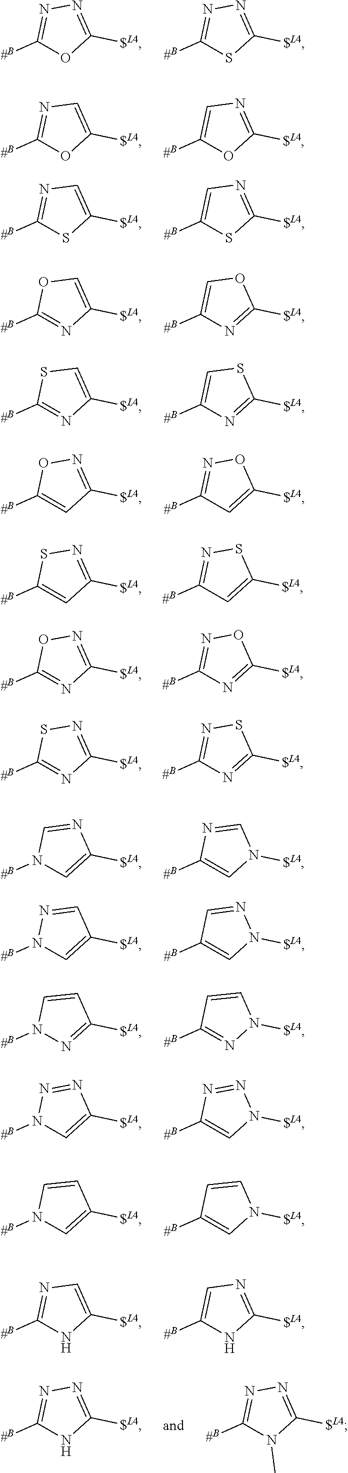

A is selected from the group consisting of:
   C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
   8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
   C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
   8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
provided that when L² is #^{L1}—N(R^{L2})—C(O)—$^{L3}, then L¹ is selected from the group consisting of a bond, #^{A}—(C₁-C₆ alkylene)—$^{L2}, #^{A}—O—$^{L2}, #^{A}—O—(C₁-C₆ alkylene)—$^{L2}, and #^{A}—N(R^{L1})—$^{L2}.
   In some embodiments, D is selected from the group consisting of:

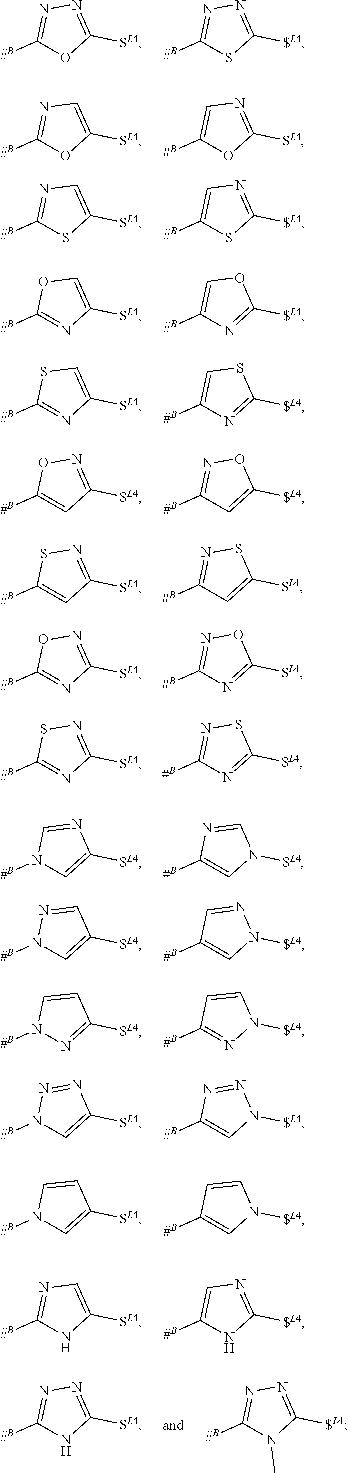

A is selected from the group consisting of:

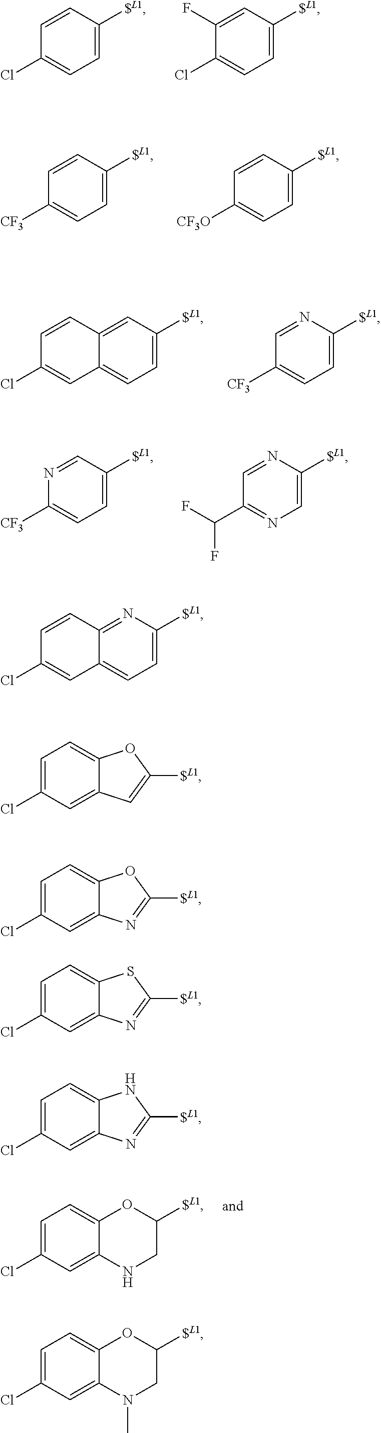

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (II), or the salt thereof, is a compound of is a compound of formula (II-a):

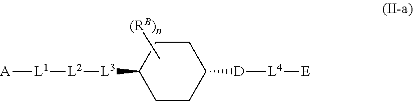

or a pharmaceutically acceptable salt thereof. In some embodiments, D is selected from the group consisting of:

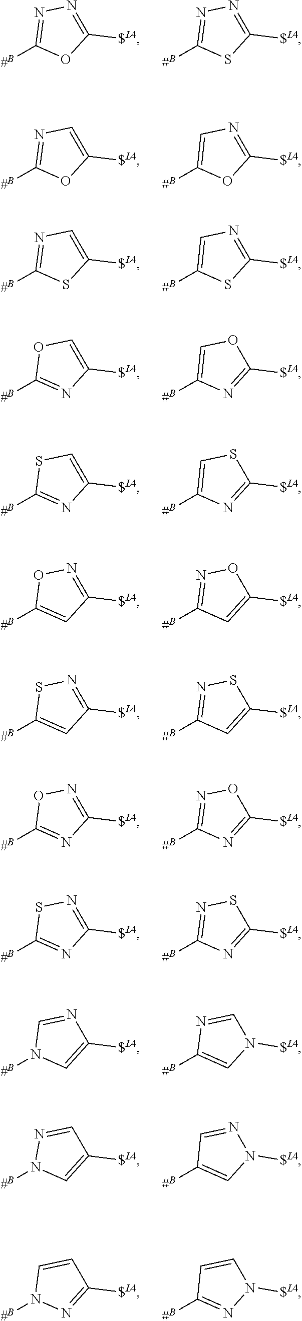

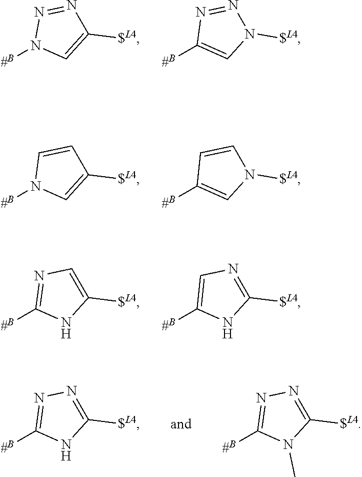

In some embodiments, D is selected from the group consisting of:

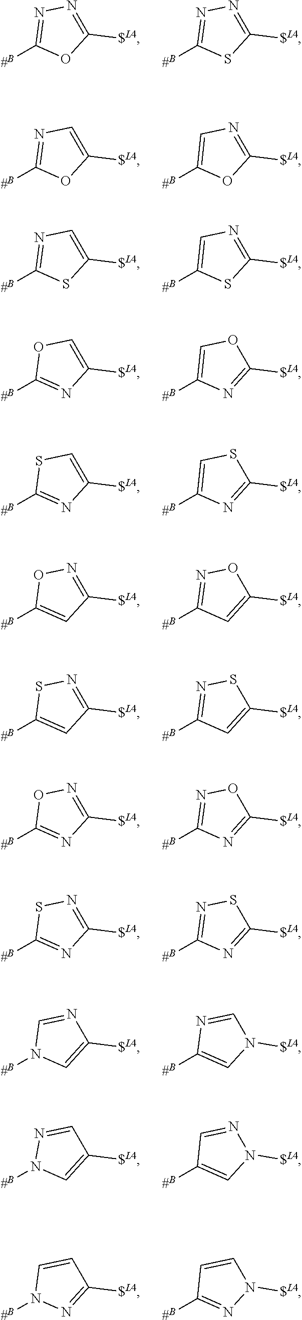

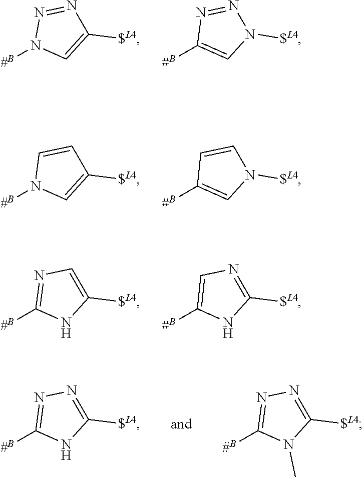

A is selected from the group consisting of:
   C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH₁(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₁ haloalkyl)S(O)₂(C₁-C₆ haloalkyl), and optionally further substituted with 1, 2, 3, 4, 5, 6, 7, or 8 R^{A} substituents;
   9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
   8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
   C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
   8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
n is an integer selected from the group consisting of 0, 1, 2, 3, 4, 5, 6, 7, 8, and 9; and
provided that when L² is #^{L1}—N(R^{L2})—C(O)—$^{L3}, then L¹ is selected from the group consisting of a bond, #^{A}—(C₁-C₆ alkylene)—$^{L2}, #^{A}—O—$^{L2}, #^{A}—O—(C₁-C₆ alkylene)—$^{L2}, and #^{A}—N(R^{L1})—$^{L2}.
   In some embodiments, D is selected from the group consisting of:

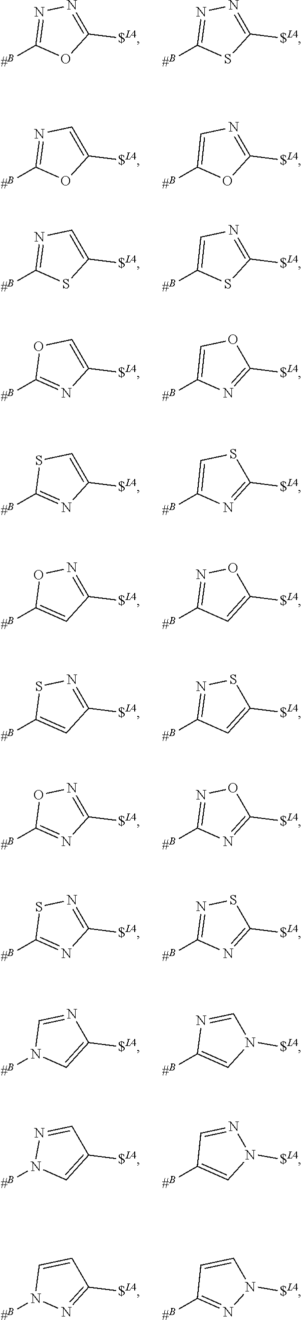

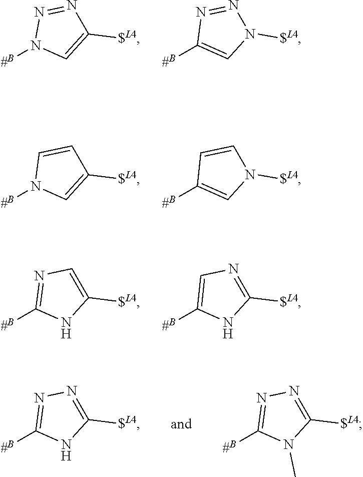

A is selected from the group consisting of:

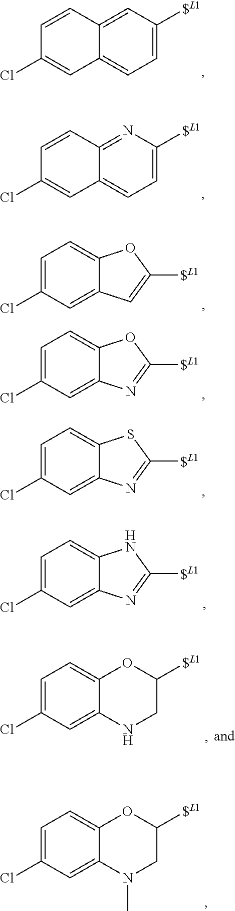

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (II-a), or the salt thereof, is a compound of is a compound of formula (II-a-i):

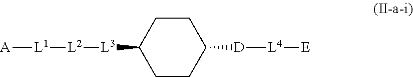

or a pharmaceutically acceptable salt thereof. In some embodiments, D is selected from the group consisting of:

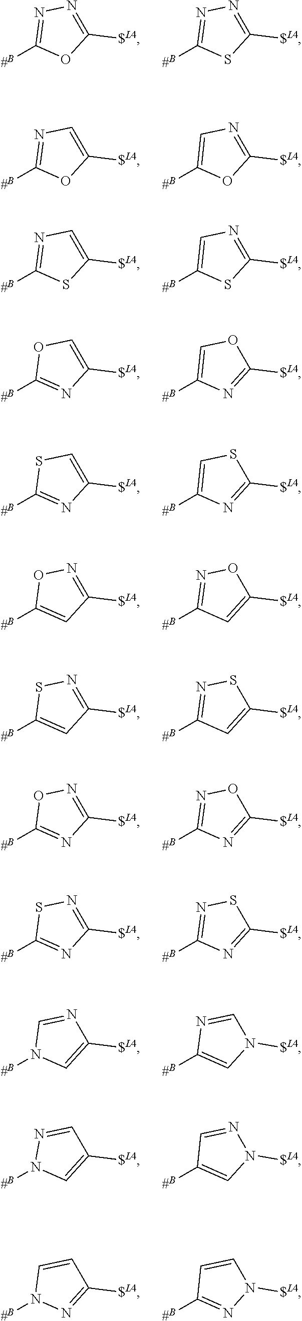

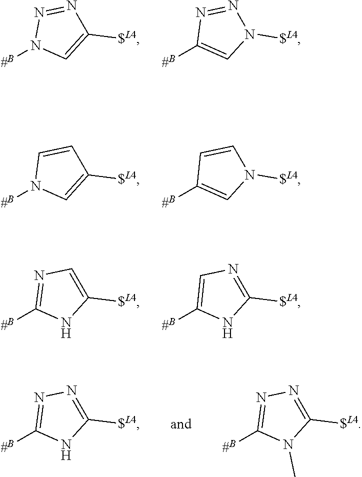

In some embodiments, D is selected from the group consisting of:

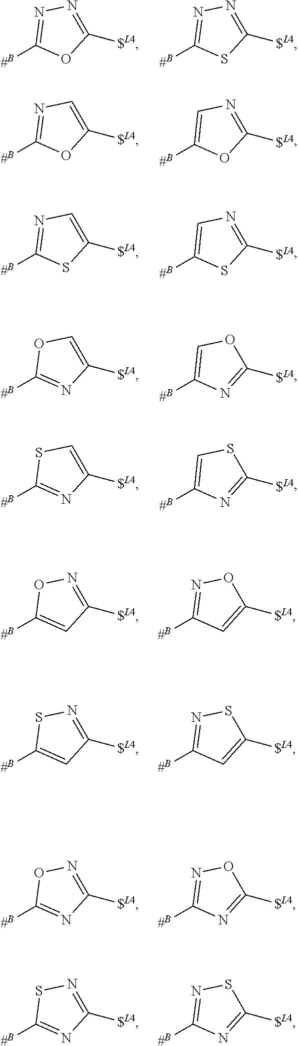

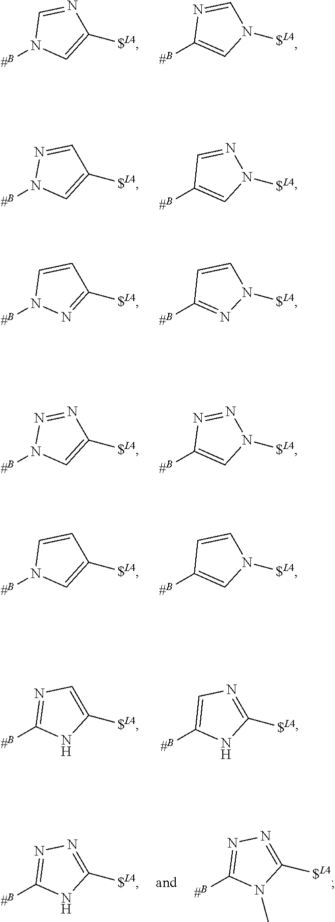

A is selected from the group consisting of:
   C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH₁(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 RA substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH₁(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
   8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
   C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
   8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
provided that when L² is #^{L1}—N(R^{L2})—C(O)—$^{L3}, then L¹ is selected from the group consisting of a bond, #^{A}—(C₁-C₆ alkylene)—$^{L2}, #^{A}—O—$^{L2}, #^{A}—O—(C₁-C₆ alkylene)—$^{L2}, and #^{A}—N(R^{L1})—$^{L2}.
   In some embodiments, D is selected from the group consisting of:

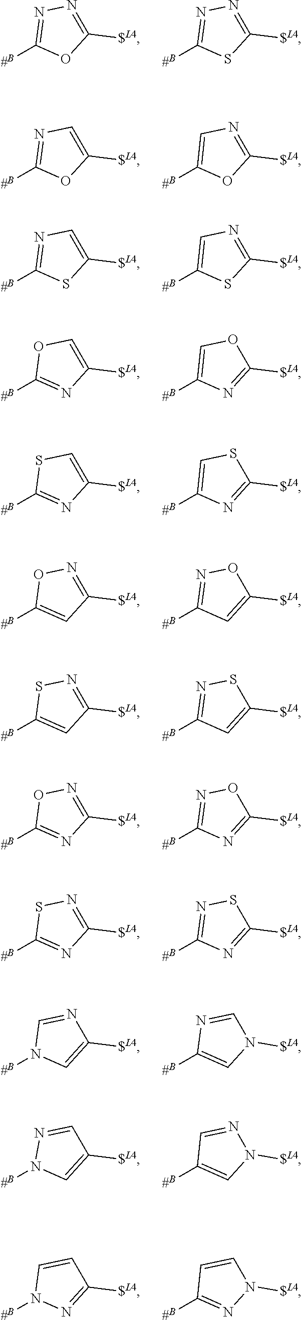

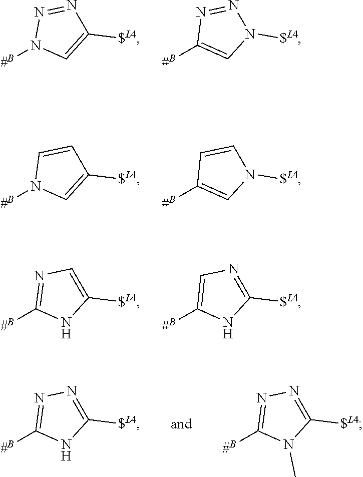

A is selected from the group consisting of:

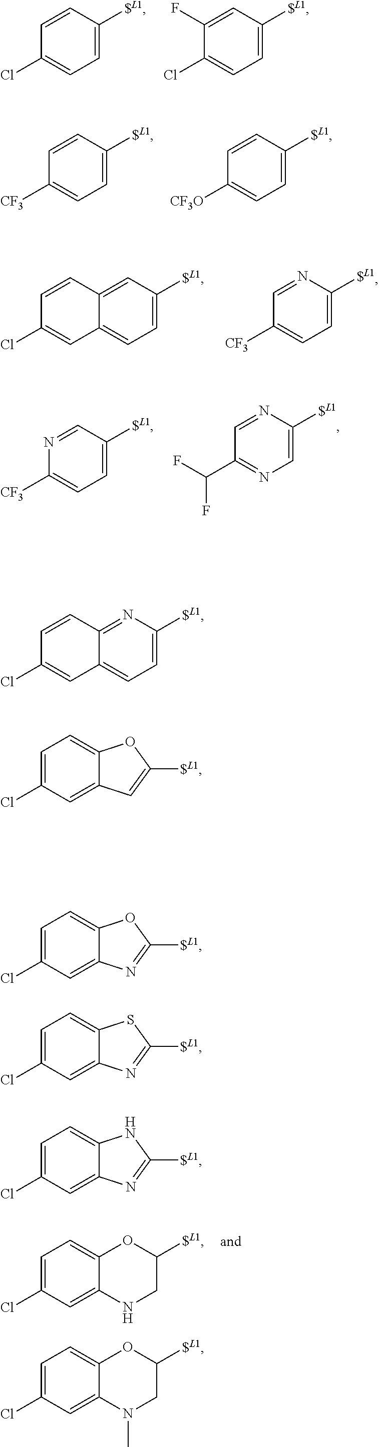

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, is a compound of is a compound of formula (III):

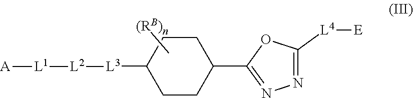

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

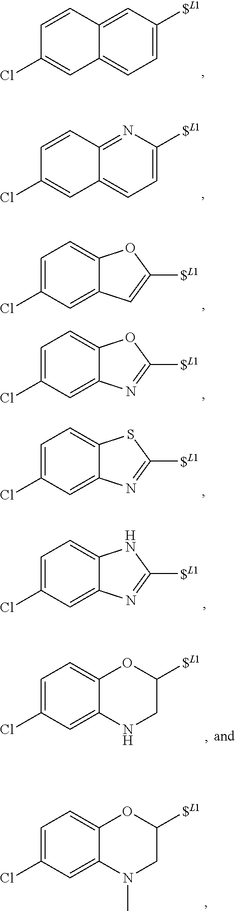

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (III), or the salt thereof, is a compound of is a compound of formula (III-i):

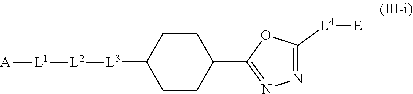

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

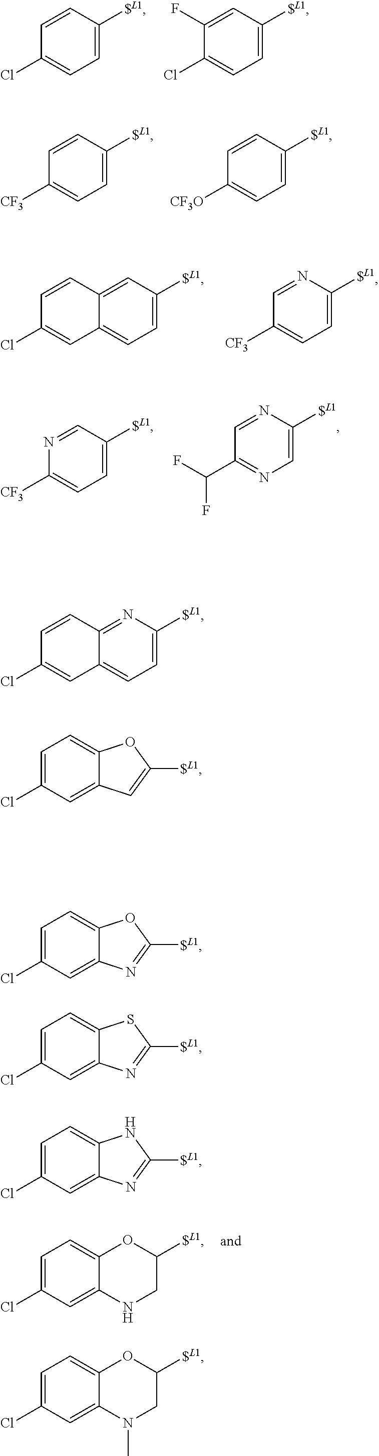

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (III), or the salt thereof, is a compound of is a compound of formula (III-a):

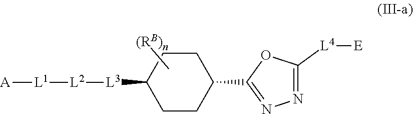

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
   5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
   8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

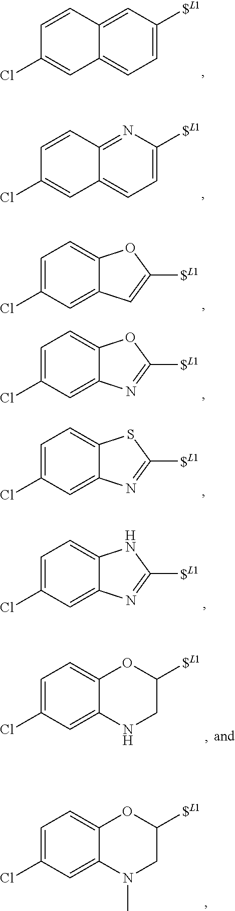

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (III-a), or the salt thereof, is a compound of formula (III-a-i):

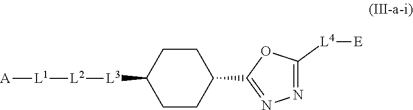

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

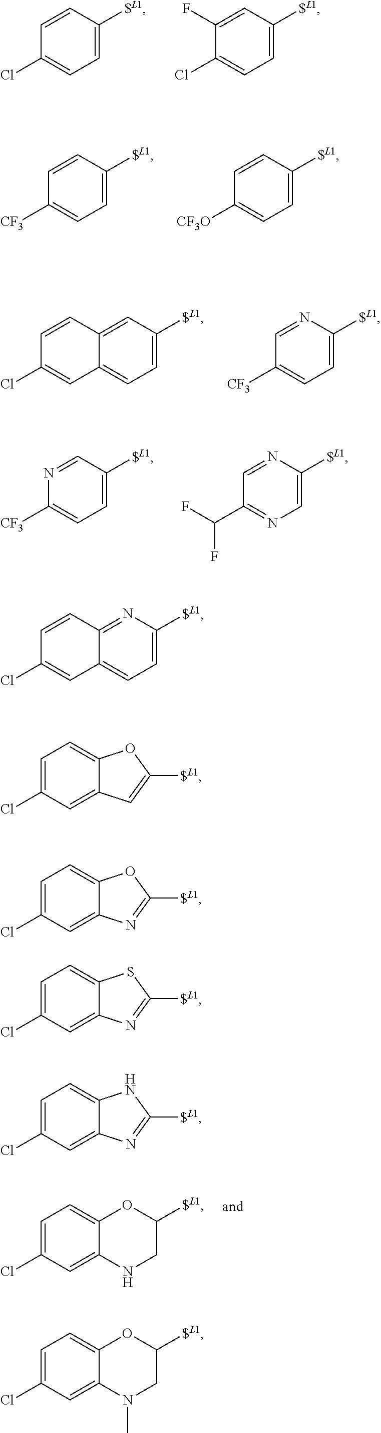

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, is a compound of is a compound of formula (IV):

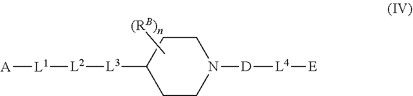

or a pharmaceutically acceptable salt thereof.

In some embodiments, the compound of formula (I), or the salt thereof, or the compound of formula (IV), or the salt thereof, is a compound of is a compound of formula (IV-i):

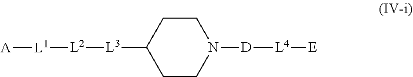

or a pharmaceutically acceptable salt thereof.

In some embodiments, the compound of formula (I), or the salt thereof, is a compound of is a compound of formula (V):

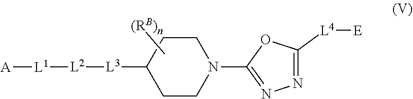

or a pharmaceutically acceptable salt thereof.

In some embodiments, the compound of formula (I), or the salt thereof or the compound of formula (V), or the salt thereof, is a compound of is a compound of formula (V-i):

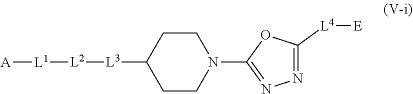

or a pharmaceutically acceptable salt thereof.

In some embodiments, the compound of formula (I), or the salt thereof, is a compound of is a compound of formula (VI):

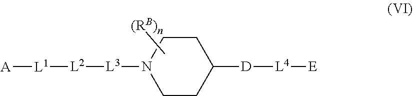

or a pharmaceutically acceptable salt thereof.

In some embodiments, the compound of formula (I), or the salt thereof, or the compound of formula (VI), or the salt thereof, is a compound of is a compound of formula (VI-i):

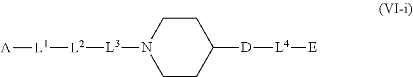

or a pharmaceutically acceptable salt thereof.

In some embodiments, the compound of formula (I), or the salt thereof, is a compound of is a compound of formula (VII):

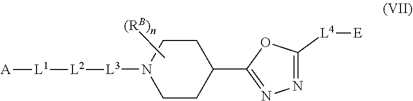

or a pharmaceutically acceptable salt thereof.

In some embodiments, the compound of formula (I), or the salt thereof, or the compound of formula (VII), or the salt thereof, is a compound of is a compound of formula (VII-i):

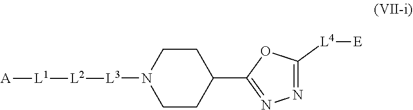

or a pharmaceutically acceptable salt thereof.

In some embodiments, the compound of formula (I), or the salt thereof, is a compound of is a compound of formula (VIII):

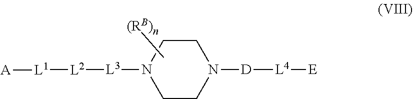

or a pharmaceutically acceptable salt thereof.

In some embodiments, the compound of formula (I), or the salt thereof, or the compound of formula (VIII), or the salt thereof, is a compound of is a compound of formula (VIII-i):

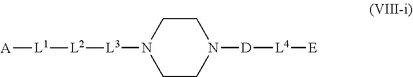

or a pharmaceutically acceptable salt thereof.

In some embodiments, the compound of formula (I), or the salt thereof, is a compound of is a compound of formula (IX):

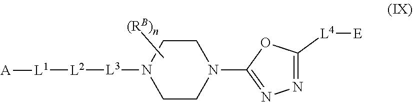

or a pharmaceutically acceptable salt thereof.

In some embodiments, the compound of formula (I), or the salt thereof, or the compound of formula (IX), or the salt thereof, is a compound of is a compound of formula (IX-i):

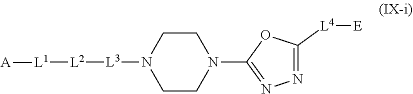

or a pharmaceutically acceptable salt thereof.

In some embodiments, the compound of formula (I), or the salt thereof, is a compound of is a compound of formula (X):

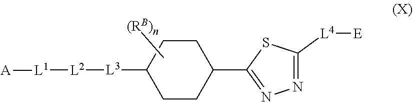

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

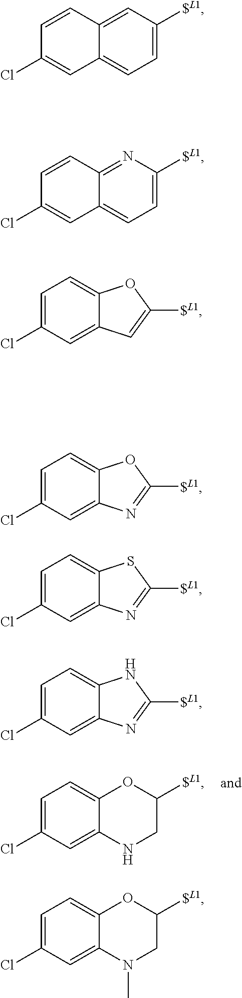

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (X), or the salt thereof, is a compound of is a compound of formula (X-i):

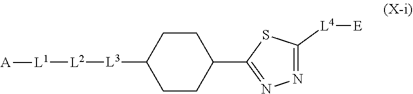

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

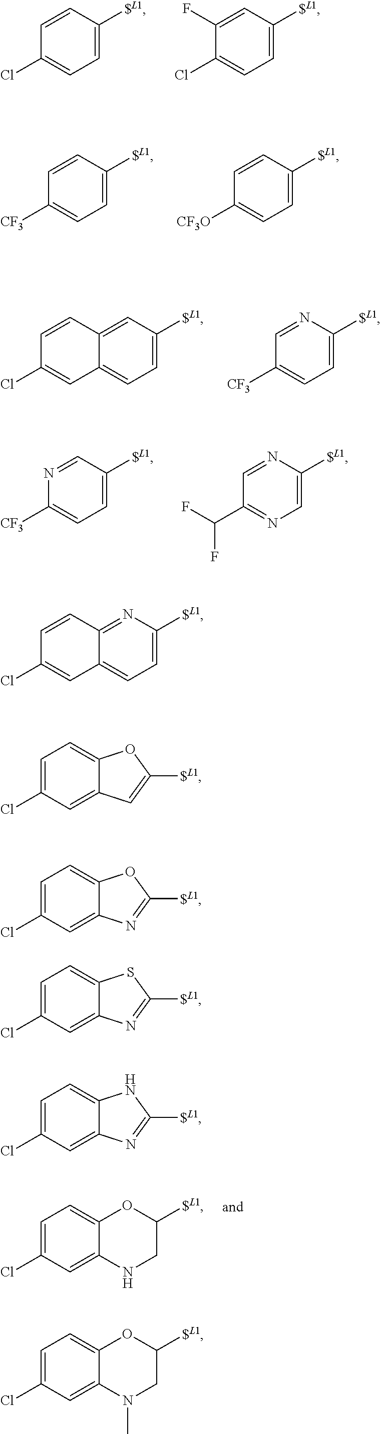

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (X), or the salt thereof, is a compound of is a compound of formula (X-a):

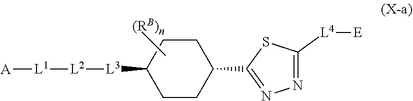

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

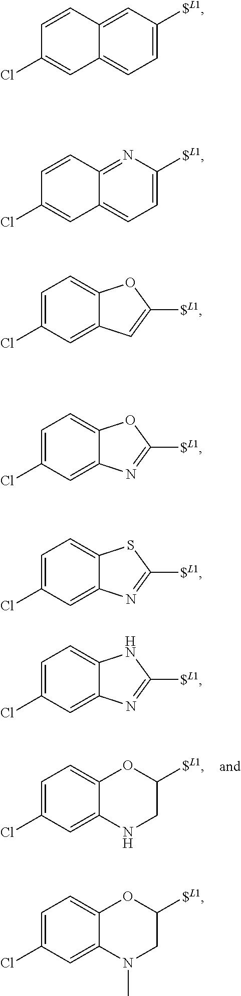

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (X-a), or the salt thereof, is a compound of is a compound of formula (X-a-i):

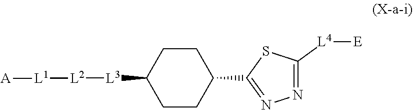

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

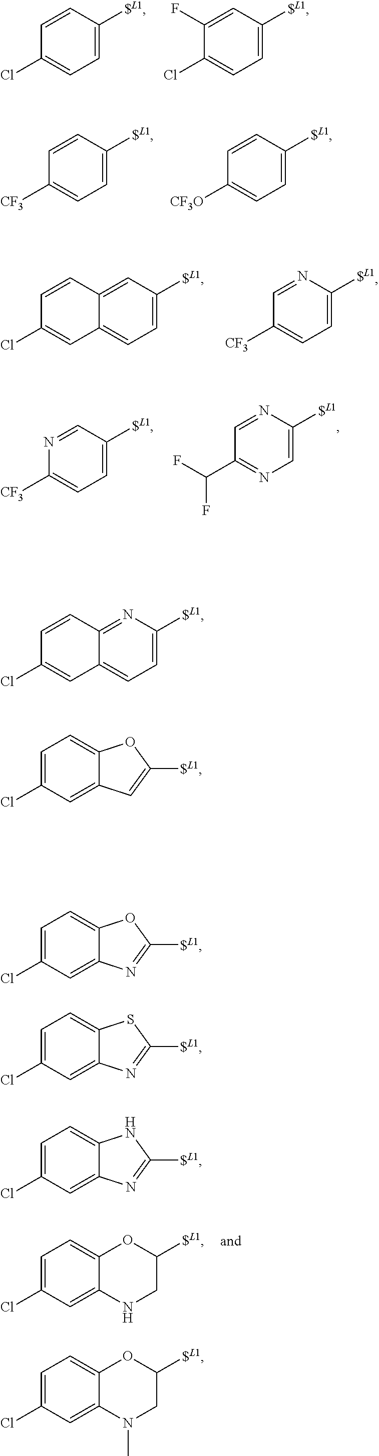

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, is a compound of is a compound of formula (XI):

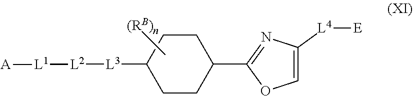

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

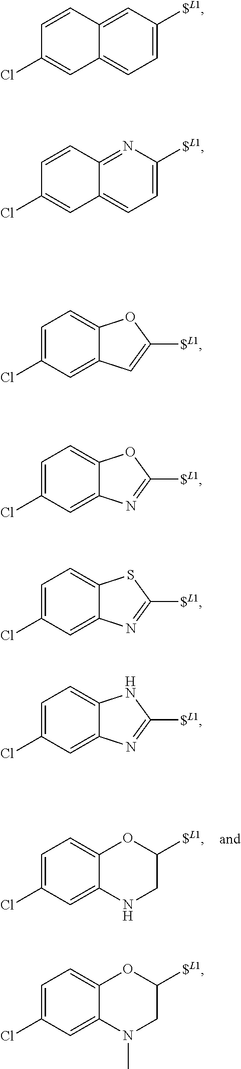

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (XI), or the salt thereof, is a compound of is a compound of formula (XI-i):

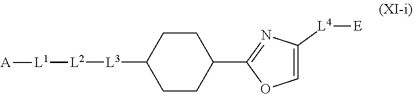

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

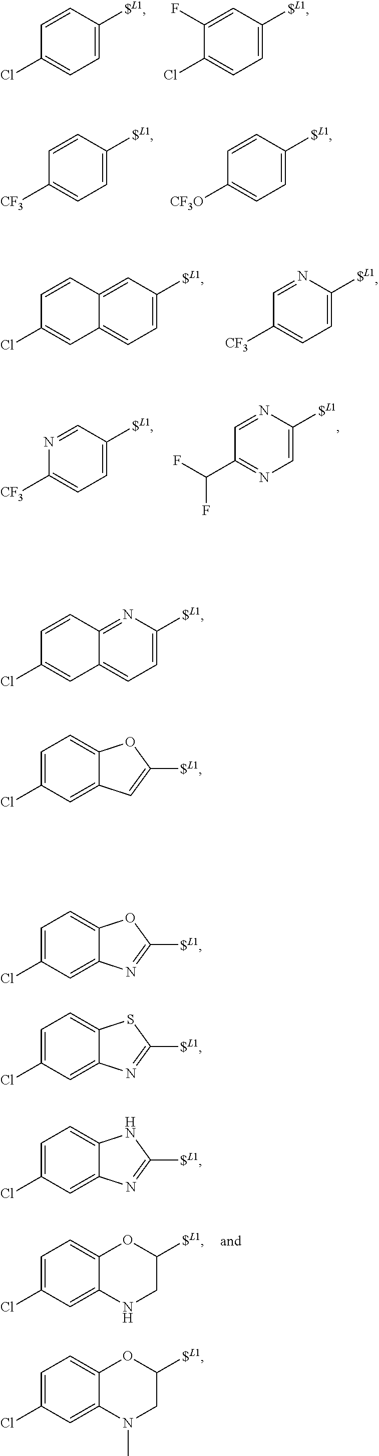

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (XI), or the salt thereof, is a compound of is a compound of formula (XI-a):

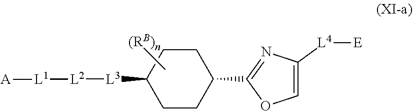

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

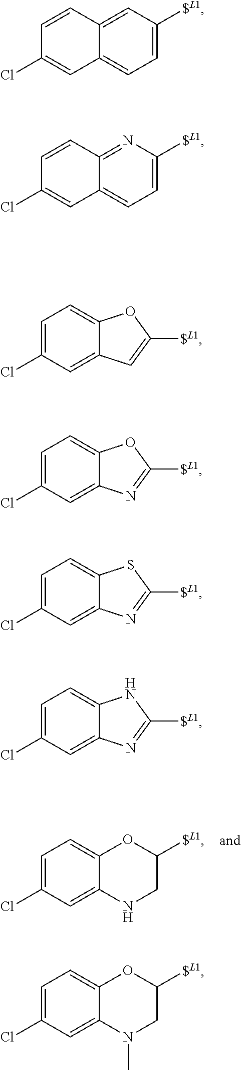

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (XI-a), or the salt thereof, is a compound of is a compound of formula (XI-a-i):

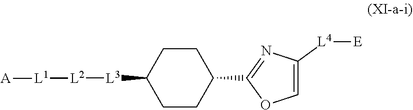

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

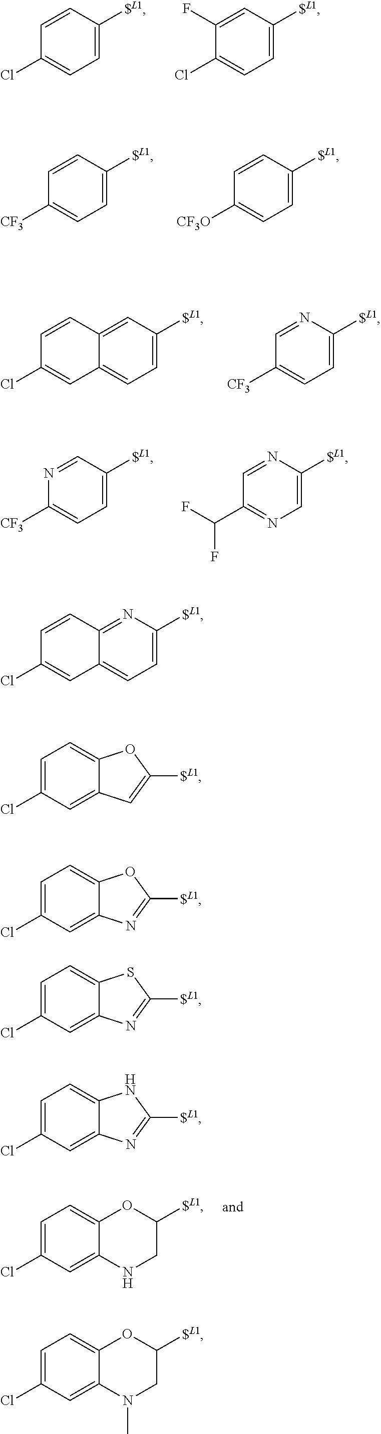

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, is a compound of is a compound of formula (XII):

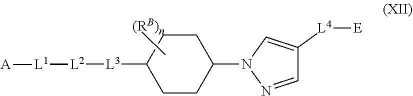

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8, and 9.
   In some embodiments, A is selected from the group consisting of:

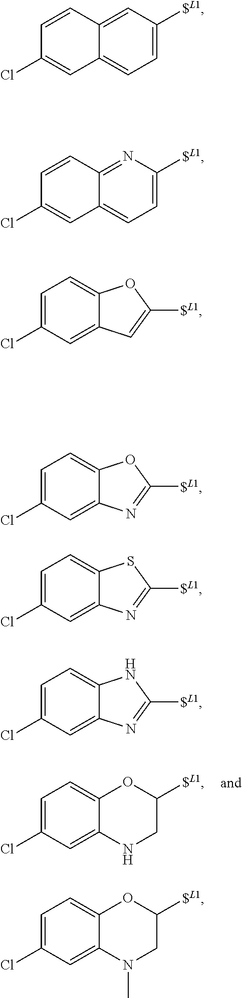

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (XII), or the salt thereof, is a compound of is a compound of formula (XII-i):

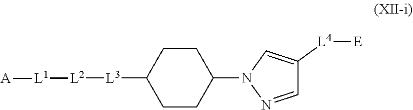

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

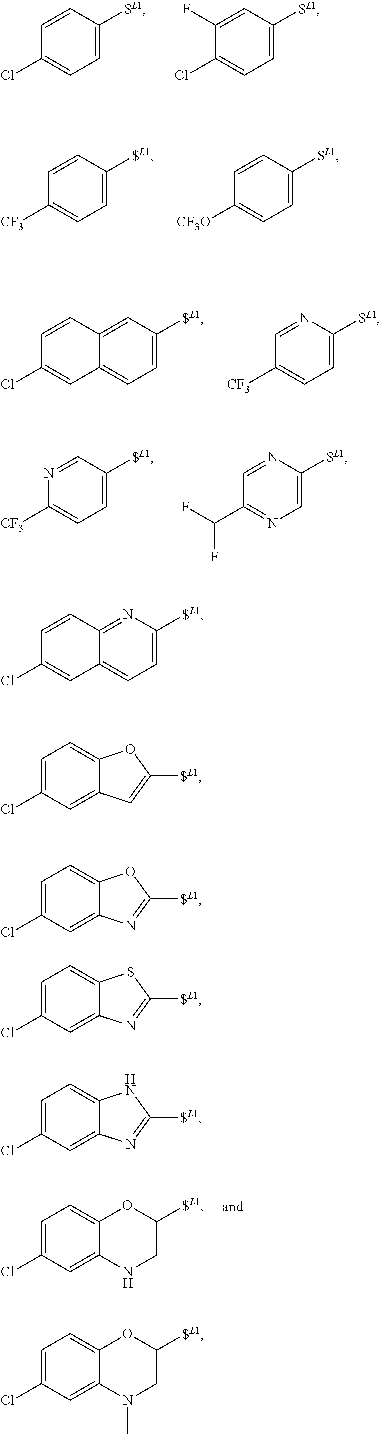

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (XII), or the salt thereof, is a compound of is a compound of formula (XII-a):

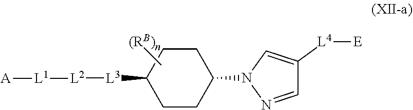

or a pharmaceutically acceptable salt thereof. In some embodiments, A is selected from the group consisting of:
C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
n is an integer selected from the group consisting of 0, 1, 2, 3, 4, 5, 6, 7, 8 and 9.
   In some embodiments, A is selected from the group consisting of:

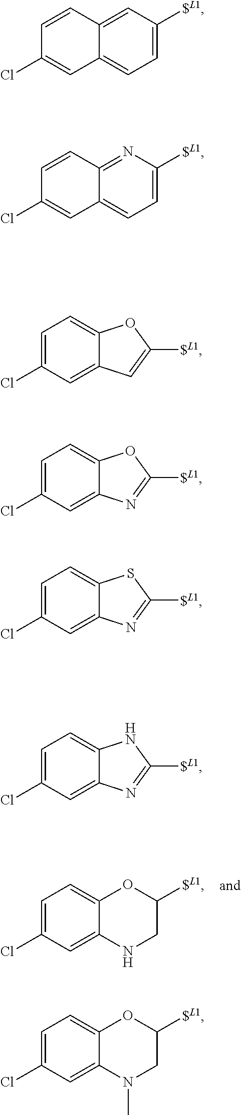

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments, the compound of formula (I), or the salt thereof, or the compound of formula (XII-a), or the salt thereof, is a compound of is a compound of formula (XII-a-i):

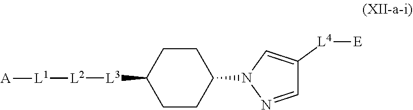

or a pharmaceutically acceptable salt thereof. In some embodiments, A is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents; and
8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.
   In some embodiments, A is selected from the group consisting of:

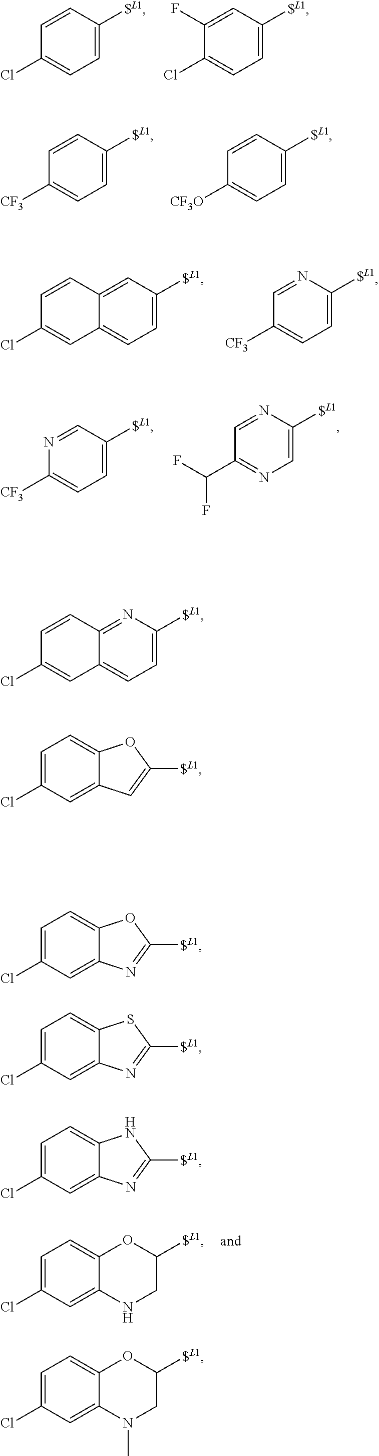

wherein $^{L1} represents the attachment point to L¹;
E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), or the salts thereof, A is selected from the group consisting of:
A is selected from the group consisting of:
   C₁₀-C₁₄ aryl optionally substituted with 1, 2, 3, 4, 5, 6, 7, 8, or 9 R^{A} substituents;
   9-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}O—$^{L2}, #^{A}—O—(C₁-C₆ alkylene)—$^{L2}, #^{A}—C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of:

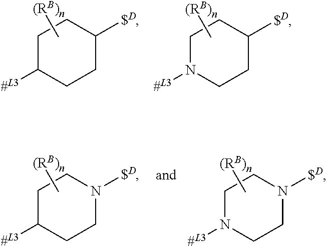

wherein
#^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
   C₄-C₁₄ cycloalkyl optionally substituted with 1, 2, 3, 4, 5, 6, 7, 8, or 9 R^{E} substituents;
   3-14 membered heterocycloalkyl optionally substituted with 1, 2, 3, 4, 5, 6, 7, 8, or 9 R^{E} substituents;
   C₆-C₁₄ aryl substituted with 1, 2, 3, 4, 5, 6, 7, 8, or 9 R^{E} substituents;
   5-14 membered heteroaryl substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)-NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)-NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)-NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)-NH—(C₁-C₆ haloalkyl);
R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
n is an integer selected from the group consisting of 0, 1, 2, 3, 4, 5, 6, 7, 8, and 9.

In another aspect, provided is a compound of formula (XIII)

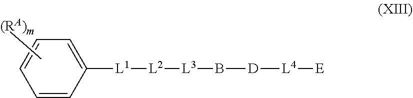

or a pharmaceutically acceptable salt thereof,
wherein:
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of:

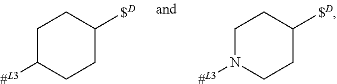

wherein #^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
   C₄-C₁₄ cycloalkyl optionally substituted with 1, 2, 3, 4, 5, 6, 7, 8, or 9 R^{E} substituents;
   3-14 membered heterocycloalkyl optionally substituted with 1, 2, 3, 4, 5, 6, 7, 8, or 9 R^{E} substituents;
   C₆-C₁₄ aryl substituted with a substituent selected from the group consisting of halogen, C₁-C₆ haloalkyl, and O(C₁-C₆ haloalkyl), and optionally further substituted with 1, 2, 3, 4, 5, 6, 7, or 8R^{E} substituents;
   5-14 membered heteroaryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
m is an integer selected from the group consisting of 0, 1, 2, 3, 4, 5, 6, 7, 8, and 9; and
provided that
(i) when B is

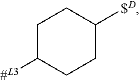

### D is

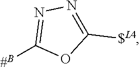

and E is cyclobutyl substituted with 1, 2, 3, 4, 5, 6, 7, 8, or 9 R^{E} substituents, then L⁴ is #^{D}—CH₂—O—$^{E}; or
(ii) when B is

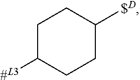

L³ is —CH₂—, and L² is #^{L1}—C(O)—N(R^{L2})—$^{L3}, then L¹ is selected from the group consisting of a bond, C₁-C₆ alkylene, #^{A}—O—(C₁-C₆ alkylene)—$^{L2}, and #^{A}—N(R^{L1})—(C₁-C₆ alkylene)—$^{L2}, wherein #^{A} represents the attachment point to A and $^{L2} represents the attachment point to L².

In some embodiments, the compound of formula (XIII), or the salt thereof, is a compound of formula (XIV)

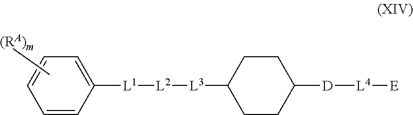

or a pharmaceutically acceptable salt thereof;
wherein L¹, L², L³, L⁴, D, E, R^{A}, and m are as defined for the compound of formula (XIII).

In some embodiments, the compound of formula (XIII), or the salt thereof, or the compound of formula (XIV), or the salt thereof, is a compound of formula (XIV-i)

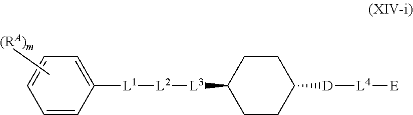

or a pharmaceutically acceptable salt thereof;
wherein L¹, L², L³, L⁴, D, E, R^{A}, and m are as defined for the compound of formula (XIII).
wherein L¹, L², L³, L⁴, D, E, R^{A}, and m are as defined for the compound of formula (XIII).

In some embodiments, the compound of formula (XIII), or the salt thereof, is a compound of formula (XV)

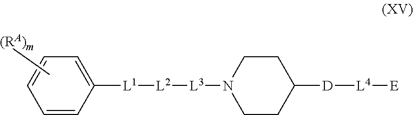

or a pharmaceutically acceptable salt thereof;
wherein L¹, L², L³, L⁴, D, E, R^{A}, and m are as defined for the compound of formula (XIII).

In another aspect, provided is a compound of formula (XVI)

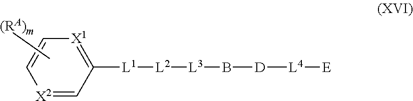

or a pharmaceutically acceptable salt thereof,
wherein:
X¹ and X² are each independently N or CH, provided at least one of X¹ and X² is N;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
   wherein R^{L2} is H, C₁-C₆ alkyl, or C₁-C₆ haloalkyl;
L³ is a bond, —N(R^{L3})—, or —CH₂—;
   wherein R^{L3} is H, C₁-C₆ alkyl, or C₁-C₆ haloalkyl;
B is selected from the group consisting of:

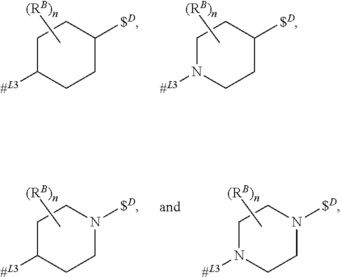

wherein #^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
   C₄-C₁₄ cycloalkyl optionally substituted with 1, 2, 3, 4, 5, 6, 7, 8, or 9 R^{E} substituents;
   3-14 membered heterocycloalkyl optionally substituted with 1, 2, 3, 4, 5, 6, 7, 8, or 9 R^{E} substituents;
   C₆-C₁₄ aryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents;
   5-14 membered heteroaryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)-NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)-NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)-NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)-NH—(C₁-C₆ haloalkyl);
R^{A}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
m and n are each independently an integer selected from the group consisting of 0, 1, 2, 3, 4, 5, 6, 7, 8, and 9.

In some embodiments, the compound of formula (XVI), or the salt thereof, is a compound of formula (XVII)

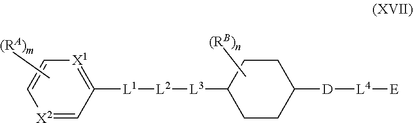

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, or the compound of formula (XVII), or the salt thereof, is a compound of formula (XVII-i)

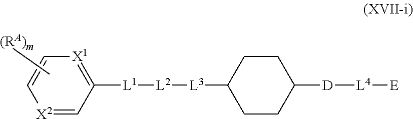

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, or the compound of formula (XVII), or the salt thereof, is a compound of formula (XVII-a)

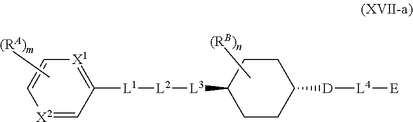

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, or the compound of formula (XVII-a), or the salt thereof, is a compound of formula (XVII-a-i)

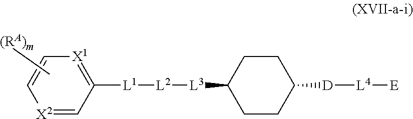

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, is a compound of is a compound of formula (XVIII):

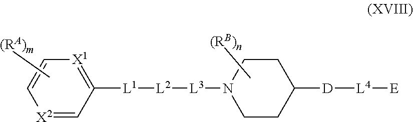

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, or the compound of formula (XVIII), or the salt thereof, is a compound of is a compound of formula (XVIII-i):

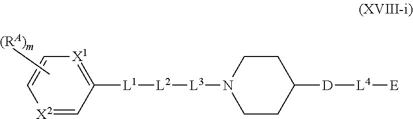

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, is a compound of is a compound of formula (XIX):

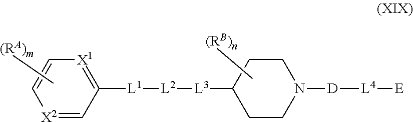

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, or the compound of formula (XIX), or the salt thereof, is a compound of is a compound of formula (XIX-i):

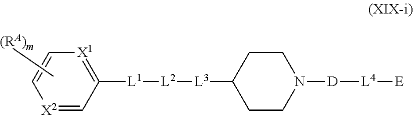

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, is a compound of is a compound of formula (XX):

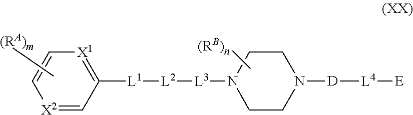

or a pharmaceutically acceptable salt thereof;
wherein L¹, L², L³, L⁴, D, E, R^{A}, R^{B}, X¹, X², m, and n are as defined for the compound of formula (XVI).

In some embodiments, the compound of formula (XVI), or the salt thereof, or the compound of formula (XX), or the salt thereof, is a compound of is a compound of formula (XIX-i):

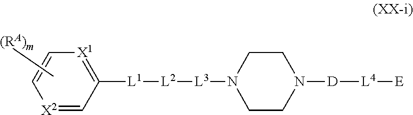

or a pharmaceutically acceptable salt thereof;
wherein L¹, L², L³, L⁴, D, E, R^{A}, R^{B}, X¹, X², m, and n are as defined for the compound of formula (XVI).

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L³ is a bond or —CH₂—. In some embodiments, L³ is a bond. In some embodiments, L³ is —CH₂—.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L² is #^{L1}—C(O)—N(R^{L2})—$^{L3}, wherein #^{L} represents the attachment point to L¹ and $^{L3} represents the attachment point to L³. In some embodiments, L² is #^{L1}—C(O)—NH—$^{L3}, wherein #^{L1} represents the attachment point to L¹ and $^{L3} represents the attachment point to L³.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L² is #^{L1}—N(R^{L2})—C(O)—$^{L3}, wherein #^{L} represents the attachment point to L¹ and $^{L3} represents the attachment point to L³. In some embodiments, L² is #^{L1}—NH—C(O)—$^{L3}, wherein #^{L1} represents the attachment point to L¹ and $^{L3} represents the attachment point to L³.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L¹ is selected from the group consisting of a bond, #^{A}—CH₂—$^{L2}, #^{A}—C(CH₃)₂—$^{L2}, #^{A}—CH₂—CH₂—$^{L2}, #^{A}—O—$^{L2}, #^{A}—O—CH₂—$^{L2}, and #^{A}—N(R^{L1})—$^{L2} wherein #^{A} represents the attachment point to A and $^{L2} represents the attachment point to L². In some embodiments, L¹ is a bond. In some embodiments, L¹ is #^{A}—CH₂—$^{L2}, wherein #^{A} represents the attachment point to A and $^{L2} represents the attachment point to L². In some embodiments, L¹ is #^{A}—C(CH₃)₂—$^{L2}, wherein #^{A} represents the attachment point to A and $^{L2} represents the attachment point to L². In some embodiments, L¹ is #^{A}—CH₂—CH₂—$^{L2}, wherein #^{A} represents the attachment point to A and $^{L2} represents the attachment point to L². In some embodiments, L¹ is #^{A}—O—$^{L2} wherein #^{A} represents the attachment point to A and $^{L2} represents the attachment point to L². In some embodiments, L¹ is #^{A}—O—CH₂—$^{L2}, wherein #^{A} represents the attachment point to A and $^{L2} represents the attachment point to L². In some embodiments, L¹ is #^{A}—N(R^{L1})—$^{L2} wherein #^{A} represents the attachment point to A and $^{L2} represents the attachment point to L². In some embodiments, L¹ is #^{A}—NH—$^{L2}, wherein #^{A} represents the attachment point to A and $^{L2} represents the attachment point to L².

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, A is selected from the group consisting of:

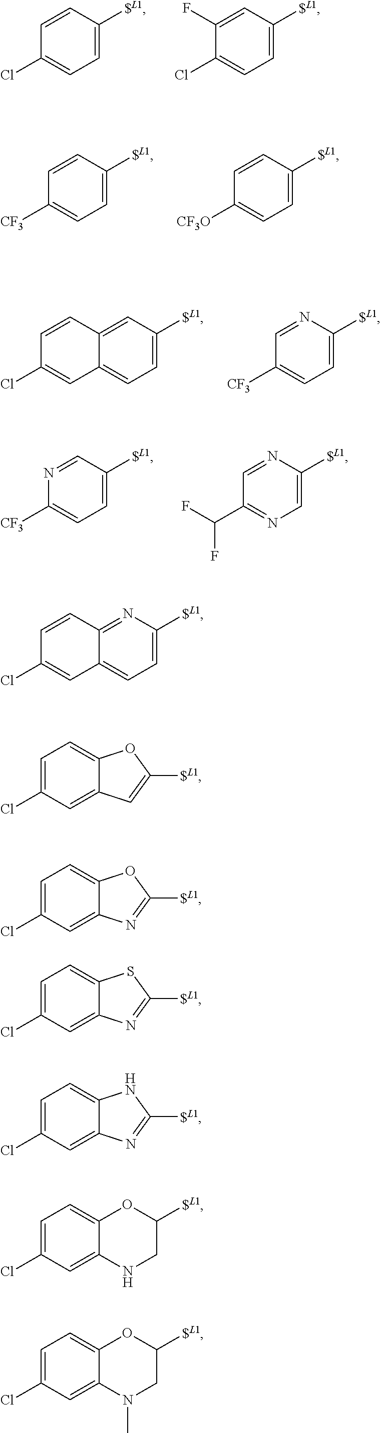

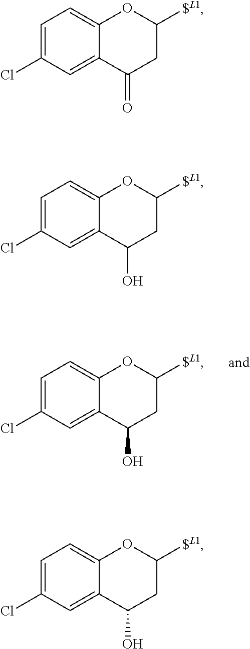

wherein $^{L1} represents the attachment point to L¹. In some embodiments, A is selected from the group consisting of:

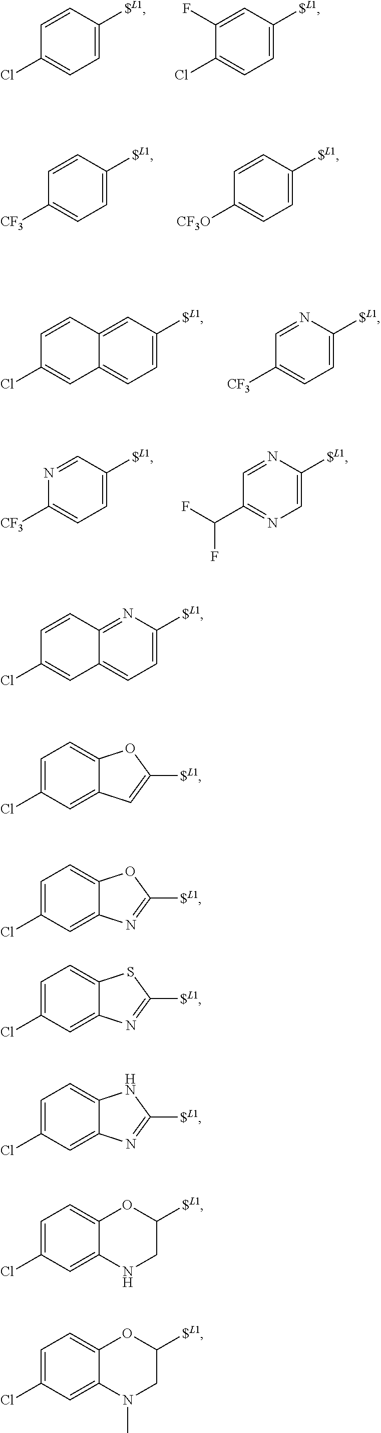

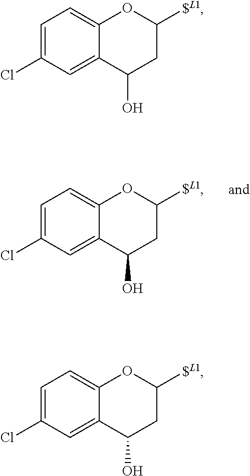

wherein $^{L1} represents the attachment point to L¹. In some embodiments, A is selected from the group consisting of:

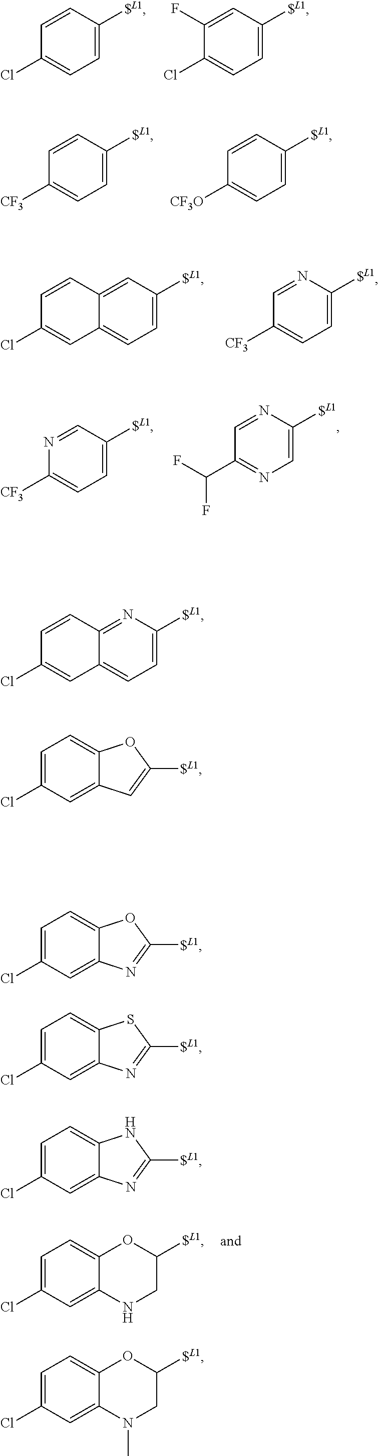

wherein $^{L1} represents the attachment point to L¹. In some embodiments, A is selected from the group consisting of:

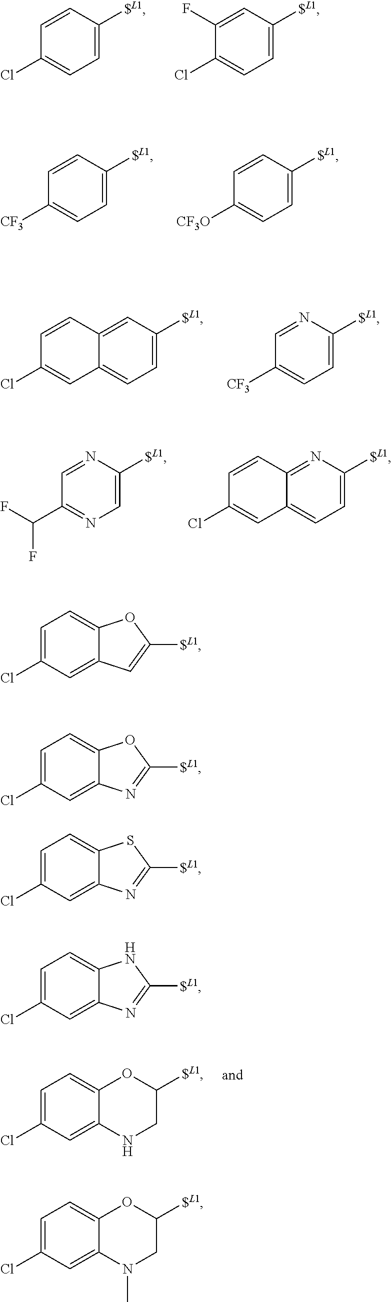

wherein $^{L1} represents the attachment point to L¹. In some embodiments, A is

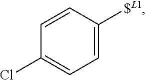

wherein $^{L1} represents the attachment point to L¹. In some embodiments, A is

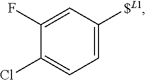

wherein $^{L1} represents the attachment point to L¹. In some embodiments, A is

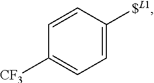

wherein $^{L1} represents the attachment point to L¹. In some embodiments, A is

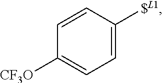

wherein $^{L1} represents the attachment point to L¹. In some embodiments, A is

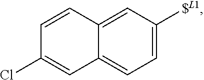

wherein $^{L1} represents the attachment point to L¹. In some embodiments, A is

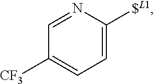

wherein $^{L1} represents the attachment point to L¹. In some embodiments, A is

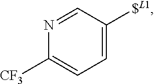

wherein $^{L1} represents the attachment point to L¹. In some embodiments, A is

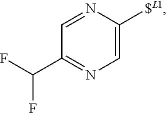

wherein $^{L1} represents the attachment point to L¹. In some embodiments, A

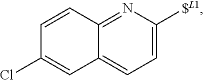

wherein $^{L1} represents the attachment point to L¹. In some embodiments, A is

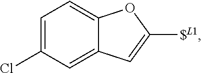

wherein $^{L1} represents the attachment point to L¹. In some embodiments, A is

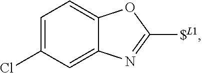

wherein $^{L1} represents the attachment point to L¹. In some embodiments, A is

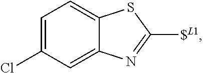

wherein $^{L1} represents the attachment point to L¹. In some embodiments, A is

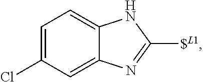

wherein $^{L1} represents the attachment point to L¹. In some embodiments, A is

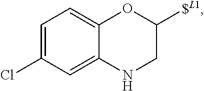

wherein $^{L1} represents the attachment point to L¹. In some embodiments, A is

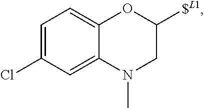

wherein $^{L1} represents the attachment point to L¹. In some embodiments, A is

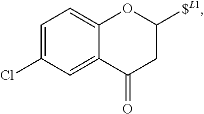

wherein $^{L1} represents the attachment point to L¹. In some embodiments, A is

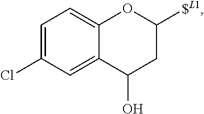

wherein $^{L1} represents the attachment point to L¹. In some embodiments, A is

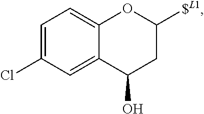

wherein $^{L1} represents the attachment point to L¹. In some embodiments, A is

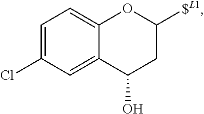

wherein $^{L1} represents the attachment point to L¹.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, A is C₆-C₁₄ aryl optionally substituted with 1, 2, 3, 4, 5, 6, 7, 8, or 9 R^{A} substituents. In some embodiments, A is C₆-C₁₄ aryl substituted with 1, 2, 3, 4, 5, 6, 7, 8, or 9 R^{A} substituents. A is C₁₀-C₁₄ aryl optionally substituted with 1, 2, 3, 4, 5, 6, 7, 8, or 9 R^{A} substituents. In some embodiments, A is C₁₀-C₁₄ aryl substituted with 1, 2, 3, 4, 5, 6, 7, 8, or 9 R^{A} substituents. In some embodiments, A is C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents. In some embodiments,
A is C₁₀-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents. In some embodiments, R^{A}, independently at each occurrence, is selected from the group consisting of halogen, C₁-C₆ haloalkyl, and O(C₁-C₆ haloalkyl). In some embodiments, R^{A}, independently at each occurrence, is selected from the group consisting of chloro, fluoro, trifluoromethyl, and trifluoromethoxy. In some embodiments, A is selected from the group consisting of:

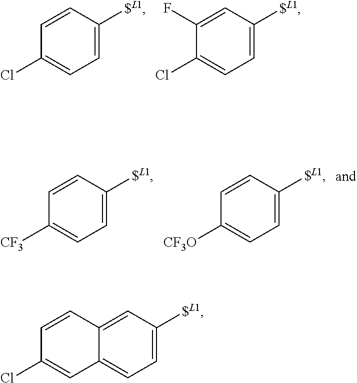

wherein $^{L1} represents the attachment point to L¹.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, A is 5-14 membered heteroaryl optionally substituted with 1, 2, 3, 4, 5, 6, 7, 8, or 9 R^{A} substituents. In some embodiments, A is 5-14 membered heteroaryl substituted with 1, 2, 3, 4, 5, 6, 7, 8, or 9 R^{A} substituents. In some embodiments, A is 5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents. In some embodiments, R^{A}, independently at each occurrence, is selected from the group consisting of halogen and C₁-C₆ haloalkyl. In some embodiments, R^{A}, independently at each occurrence, is selected from the group consisting of chloro, difluoromethyl, and trifluoromethyl. In some embodiments, A is selected from the group consisting of:

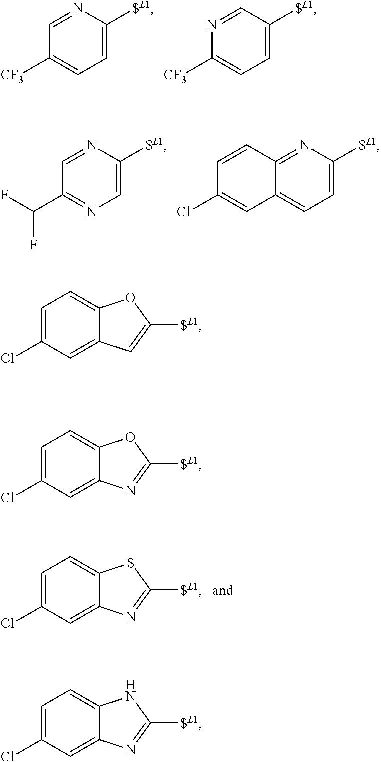

wherein $^{L1} represents the attachment point to L¹. In some embodiments,
A is selected from the group consisting of:

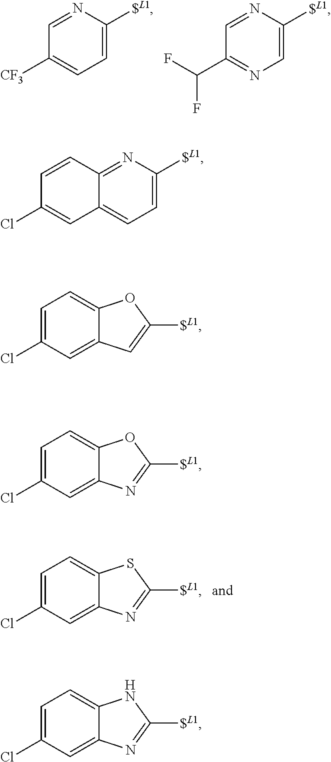

wherein $^{L1} represents the attachment point to L¹.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, A is 8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, 8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, R^{A}, independently at each occurrence, is selected from the group consisting of halogen and C₁-C₆ alkyl. In some embodiments, R^{A}, independently at each occurrence, is selected from the group consisting of chloro and methyl. In some embodiments, A is selected from the group consisting of:

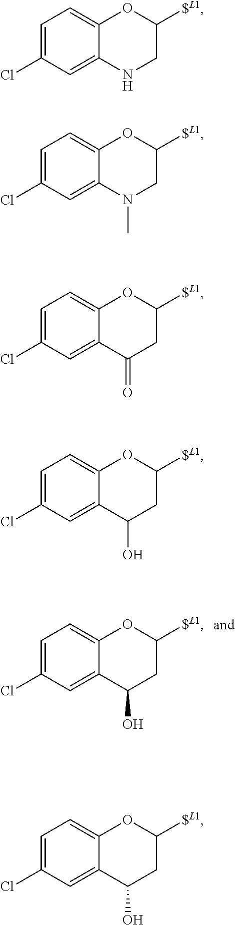

wherein $^{L1} represents the attachment point to L¹. In some embodiments, A is selected from the group consisting of:

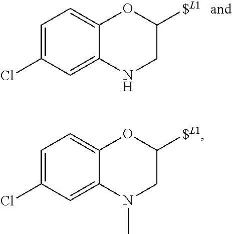

wherein $^{L1} represents the attachment point to L¹. In some embodiments, A is selected from the group consisting of:

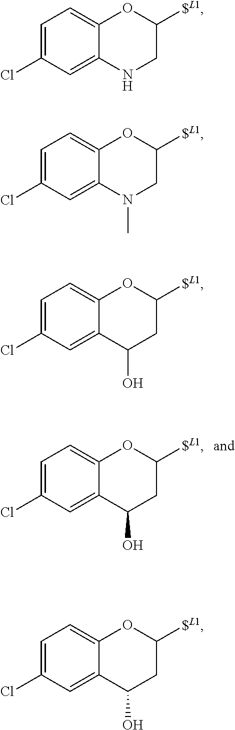

wherein $^{L1} represents the attachment point to L¹. In some embodiments, A is selected from the group consisting of:

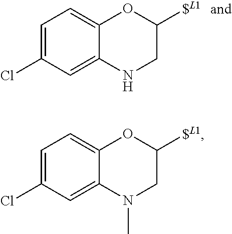

wherein $^{L1} represents the attachment point to L¹.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof:

### A is

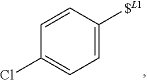

L¹ is #^{A}—O—CH₂—$^{L2}, L² is #^{L1}—C(O)—NH—$^{L3}, and L³ is a bond;

### A is

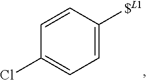

L¹ is #^{A}—O—CH₂—$^{L2}, L² is #^{L1}—C(O)—NH—$^{L3}, and L³ is —CH₂—;

### A is

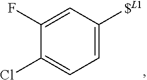

L¹ is #^{A}—O—CH₂—$^{L2}, L² is #^{L1}—C(O)—NH—$^{L3} and L³ is a bond;

### A is

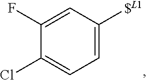

L¹ is #^{A}—O—CH₂—$^{L2}, L² is #L-C(O)—NH—$^{L3}, and L³ is —CH₂—;

### A is

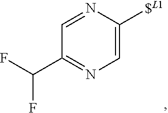

L¹ is a bond, L² is #L-C(O)—NH—$^{L3}, and L³ is a bond;

### A is

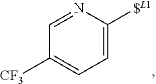

L¹ is a bond, L² is #^{L1}—C(O)—NH—$^{L3}, and L³ is —CH₂—;

### A is

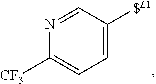

L¹ is a bond, L² is #^{L1}—C(O)—NH—$^{L3}, and L³ is —CH₂—;

### A is

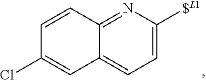

L¹ is a bond, L² is #^{L1}—C(O)—NH—$^{L3}, and L³ is a bond;

### A is

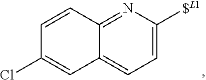

L¹ is a bond, L² is #L-C(O)—NH—$^{L3}, and L³ is —CH₂—;

### A is

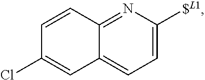

L¹ is a bond, L² is #^{L1}—NH—C(O)—$^{L3}, and L³ is a bond;

### A is

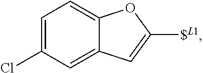

L¹ is a bond, L² is #^{L1}—C(O)—NH—$^{L3} and L³ is a bond;

### A is

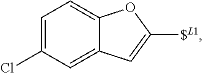

L¹ is a bond, L² is #^{L1}—C(O)—NH—$^{L3}, and L³ is —CH₂—;

### A is

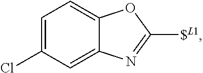

L¹ is a bond, L² is #^{L1}—NH—C(O)—$^{L3} and L³ is a bond;

### A is

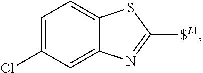

L¹ is a bond, L² is #^{L1}—NH—C(O)—$^{L3}, and L³ is a bond; or

### A is

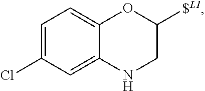

L¹ is a bond, L² is #^{L1}—C(O)—NH—$^{L3} and L³ is a bond;
wherein $^{L1} represents the attachment point to L¹, $^{L2} represents the attachment point to L², #^{L1} represents the attachment point to L¹, and $^{L3} represents the attachment point to L³.
In some embodiments, A is

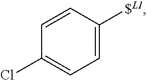

L¹ is #^{A}—O—CH₂—$^{L2}, L² is #^{L1}—C(O)—NH—$^{L3} and L³ is a bond, wherein $^{L1} represents the attachment point to L¹, #^{A} represents the attachment point to A, $^{L2} represents the attachment point to L², #^{L1} represents the attachment point to L¹, and $^{L3} represents the attachment point to L³.
In some embodiments, A is

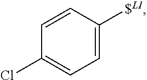

L¹ is #^{A}—O—CH₂—$^{L2}, L² is #^{L1}—C(O)—NH—$^{L3} and L³ is —CH₂—, wherein $^{L1} represents the attachment point to L¹, #^{A} represents the attachment point to A, $^{L2} represents the attachment point to L², #^{L1} represents the attachment point to L¹, and $^{L3} represents the attachment point to L³.
In some embodiments, A is

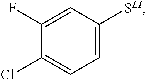

L¹ is #^{A}—O—CH₂—$^{L2}, L² is #^{L1}—C(O)—NH—$^{L3}, and L³ is a bond, wherein $^{L1} represents the attachment point to L¹, #^{A} represents the attachment point to A, $^{L2} represents the attachment point to L², #^{L1} represents the attachment point to L¹, and $^{L3} represents the attachment point to L³.
In some embodiments, A is

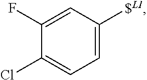

L¹ is #^{A}—O—CH₂—$^{L2}, L² is #^{L1}—C(O)—NH—$^{L3}, and L³ is —CH₂—, wherein $^{L1} represents the attachment point to L¹, #^{A} represents the attachment point to A, $^{L2} represents the attachment point to L², #^{L1} represents the attachment point to L¹, and $^{L3} represents the attachment point to L³.
In some embodiments, A is

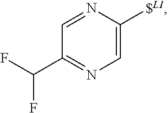

L¹ is a bond, L² is #^{L1}—C(O)—NH—$^{L3} and L³ is a bond, wherein $^{L1} represents the attachment point to L¹, $^{L2} represents the attachment point to L², #^{L1} represents the attachment point to L¹, and $^{L3} represents the attachment point to L³.
In some embodiments, A is

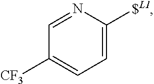

L¹ is a bond, L² is #^{L1}—C(O)—NH—$^{L3} and L³ is —CH₂—, wherein $L represents the attachment point to L¹, $L represents the attachment point to L², #^{L1} represents the attachment point to L¹, and $^{L3} represents the attachment point to L³.
In some embodiments, A is

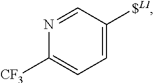

L¹ is a bond, L² is #^{L1}—C(O)—NH—$^{L3} and L³ is —CH₂—, wherein $^{L1} represents the attachment point to L¹, $^{L2} represents the attachment point to L², #^{L1} represents the attachment point to L¹, and $^{L3} represents the attachment point to L³.
In some embodiments, A is

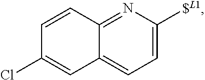

L¹ is a bond, L² is #^{L1}—C(O)—NH—$^{L3}, and L³ is a bond, wherein $^{L1} represents the attachment point to L¹, $^{L2} represents the attachment point to L², #^{L1} represents the attachment point to L¹, and $^{L3} represents the attachment point to L³.
In some embodiments, A is

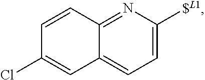

L¹ is a bond, L² is #^{L1}—C(O)—NH—$^{L3}, and L³ is —CH₂—, wherein $^{L1} represents the attachment point to L¹, $^{L2} represents the attachment point to L², #^{L1} represents the attachment point to L¹, and $^{L3} represents the attachment point to L³.
In some embodiments, A is

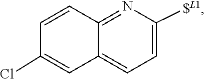

L¹ is a bond, L² is #^{L}—NH—C(O)—$^{L3}, and L³ is a bond, wherein $^{L1} represents the attachment point to L¹, $^{L2} represents the attachment point to L², #^{L1} represents the attachment point to L¹, and $^{L3} represents the attachment point to L³.
In some embodiments A is

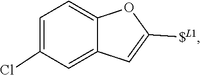

L¹ is a bond, L² is #^{L1}—C(O)—NH—$^{L3} and L³ is a bond, wherein $^{L1} represents the attachment point to L¹, $^{L2} represents the attachment point to L², #^{L1} represents the attachment point to L¹, and $^{L3} represents the attachment point to L³.
In some embodiments, A is

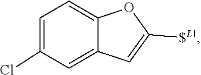

L¹ is a bond, L² is #^{L1}—C(O)—NH—$^{L3} and L³ is —CH₂—, wherein $^{L1} represents the attachment point to L¹, $^{L2} represents the attachment point to L², #^{L1} represents the attachment point to L¹, and $^{L3} represents the attachment point to L³.
In some embodiments, A is

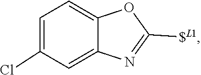

L¹ is a bond, L² is #^{L1}—NH—C(O)—$^{L3} and L³ is a bond, wherein $^{L1} represents the attachment point to L¹, $^{L2} represents the attachment point to L², #^{L1} represents the attachment point to L¹, and $^{L3} represents the attachment point to L³.
In some embodiments, A is

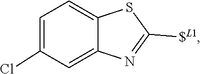

L¹ is a bond, L² is #^{L1}—NH—C(O)—$^{L3}, and L³ is a bond, wherein $^{L1} represents the attachment point to L¹, $^{L2} represents the attachment point to L², #^{L1} represents the attachment point to L¹, and $^{L3} represents the attachment point to L³.
In some embodiments, A is

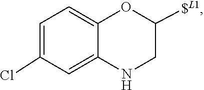

L¹ is a bond, L² is #^{L1}—C(O)—NH—$^{L3} and L³ is a bond, wherein $^{L1} represents the attachment point to L¹, $^{L2} represents the attachment point to L², #^{L1} represents the attachment point to L¹, and $^{L3} represents the attachment point to L³.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L⁴ is a bond.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L⁴ is #^{D}—CH₂—$^{E}, wherein #^{D} represents the attachment point to D and $^{E} represents the attachment point to E.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L⁴ is #^{D}—CH₂—O—$^{E}, wherein #^{D} represents the attachment point to D and $^{E} represents the attachment point to E.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, E is C₃-C₁₄ cycloalkyl optionally substituted with 1, 2, 3, 4, 5, 6, 7, 8, or 9 R^{E} substituents. In some embodiments, E is C₄-C₁₄ cycloalkyl optionally substituted with 1, 2, 3, 4, 5, 6, 7, 8, or 9 R^{E} substituents. In some embodiments, R^{E}, independently at each occurrence, is selected from the group consisting of halogen, C₁-C₆ haloalkyl, OH, O(C₁-C₆ alkyl), and O(C₁-C₆ haloalkyl). In some embodiments, R^{E}, independently at each occurrence, is selected from the group consisting of fluoro, trifluoromethyl, OH, methoxy, and trifluoromethoxy. In some embodiments, E is C₃-C₁₄ cycloalkyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, R^{E}, independently at each occurrence, is selected from the group consisting of fluoro, trifluoromethyl, OH, methoxy, and trifluoromethoxy. In some embodiments, E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, R^{E}, independently at each occurrence, is selected from the group consisting of fluoro, trifluoromethyl, OH, methoxy, and trifluoromethoxy. In some embodiments, E is selected from the group consisting of:

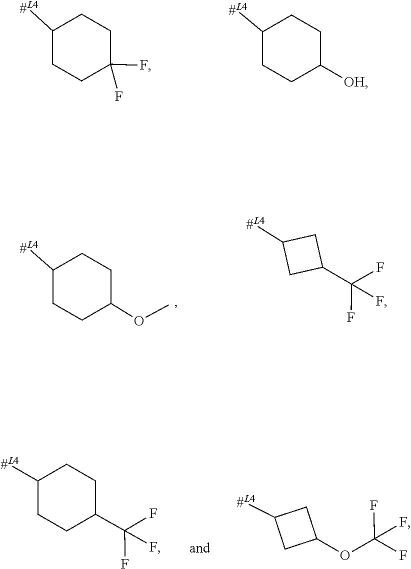

wherein #^{L4} represents the attachment point to L⁴. In some embodiments, E is

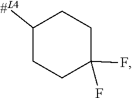

wherein #^{L4} represents the attachment point to L⁴. In some embodiments, E is

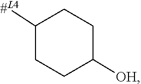

wherein #^{L4} represents the attachment point to L⁴. In some embodiments, E is

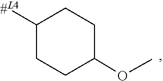

wherein #^{L4} represents the attachment point to L⁴. In some embodiments, E is

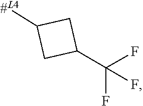

wherein #^{L4} represents the attachment point to L⁴. In some embodiments, E is

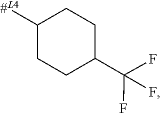

wherein #^{L4} represents the attachment point to L⁴. In some embodiments, E is

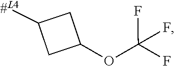

wherein #^{L4} represents the attachment point to L⁴.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, E is 3-14 heterocycloalkyl optionally substituted with 1, 2, 3, 4, 5, 6, 7, 8, or 9 R^{E} substituents. In some embodiments, R^{E}, independently at each occurrence, is selected from the group consisting of halogen, C₁-C₆ alkyl, C₁-C₆ haloalkyl, OH, O(C₁-C₆ alkyl), and O(C₁-C₆ haloalkyl). In some embodiments, R^{E}, independently at each occurrence, is selected from the group consisting of fluoro, methyl, trifluoromethyl, OH, methoxy, and trifluoromethoxy. In some embodiments, E is selected from the group consisting of:

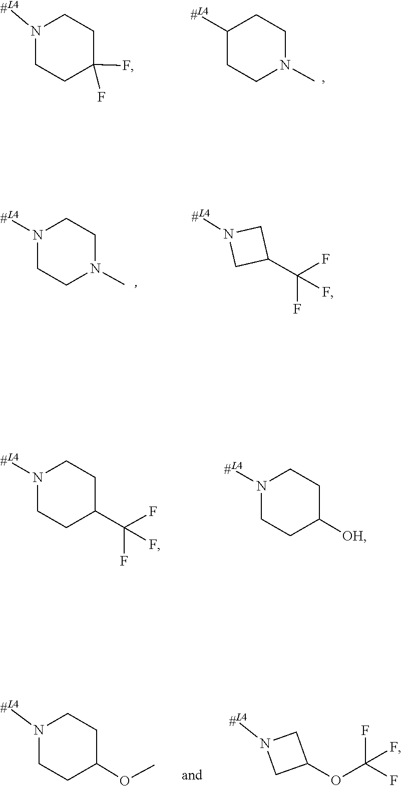

wherein #^{L4} represents the attachment point to L⁴. In some embodiments, E is

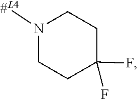

wherein #^{L4} represents the attachment point to L⁴. In some embodiments, E is

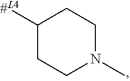

wherein #^{L4} represents the attachment point to L⁴. In some embodiments, E is

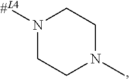

wherein #^{L4} represents the attachment point to L⁴. In some embodiments, E is

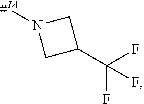

wherein #^{L4} represents the attachment point to L⁴. In some embodiments, E is

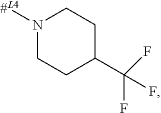

wherein #^{L4} represents the attachment point to L⁴. In some embodiments, E is

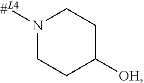

wherein #^{L4} represents the attachment point to L⁴. In some embodiments, E is

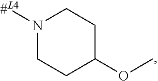

wherein #^{L4} represents the attachment point to L⁴. In some embodiments, E is

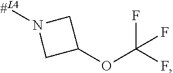

wherein #^{L4} represents the attachment point to L⁴.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, E is C₆-C₁₄ aryl optionally substituted with 1, 2, 3, 4, 5, 6, 7, 8, or 9 R^{E} substituents. In some embodiments, E is C₆-C₁₄ aryl substituted with 1, 2, 3, 4, 5, 6, 7, 8, or 9 R^{E} substituents. In some embodiments, C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, E is C₆-C₁₄ aryl substituted with a substituent selected from the group consisting of halogen, C₁-C₆ haloalkyl, and O(C₁-C₆ haloalkyl), and optionally further substituted with 1, 2, 3, 4, 5, 6, 7, or 8R^{E} substituents. In some embodiments, R^{E}, independently at each occurrence, is halogen. In some embodiments, R^{E}, independently at each occurrence, is selected from the group consisting of fluoro and chloro. In some embodiments, E is selected from the group consisting of:

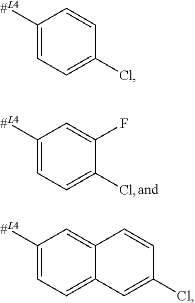

wherein #^{L4} represents the attachment point to L⁴. In some embodiments, E is

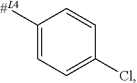

wherein #^{L4} represents the attachment point to L⁴. In some embodiments, E is

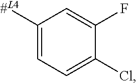

wherein #^{L4} represents the attachment point to L⁴. In some embodiments, E is

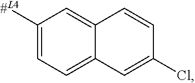

wherein #^{L4} represents the attachment point to L⁴.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, E is 5-14 membered heteroaryl optionally substituted with 1, 2, 3, 4, 5, 6, 7, 8, or 9 R^{E} substituents. In some embodiments, E is 5-14 membered heteroaryl substituted with 1, 2, 3, 4, 5, 6, 7, 8, or 9 R^{E} substituents. In some embodiments, E is 5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, E is 5-14 membered heteroaryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents. In some embodiments, R^{E}, independently at each occurrence, is selected from the group consisting of halogen and O(C₁-C₆ alkyl). In some embodiments, R^{E}, independently at each occurrence, is selected from the group consisting of chloro and methoxy. In some embodiments, E is selected from the group consisting of:

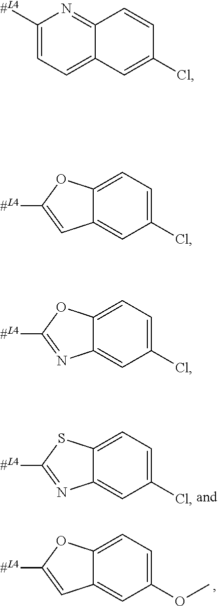

wherein #^{L4} represents the attachment point to L⁴. In some embodiments, E is

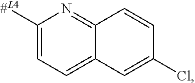

wherein #^{L4} represents the attachment point to L⁴. In some embodiments, E is

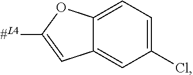

wherein #^{L4} represents the attachment point to L⁴. In some embodiments, E is

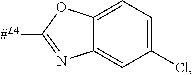

wherein #^{L4} represents the attachment point to L⁴. In some embodiments, E is

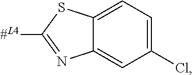

wherein #^{L4} represents the attachment point to L⁴. In some embodiments, E is

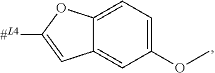

wherein #^{L4} represents the attachment point to L⁴.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, E is 8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, E is 8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, R^{E}, independently at each occurrence, is halogen. In some embodiments, R^{E}, independently at each occurrence, is chloro. In some embodiments, E is

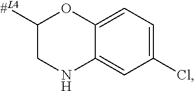

wherein #^{L4} represents the attachment point to L⁴. In some embodiments, R^{E}, independently at each occurrence, is chloro. In some embodiments, E is

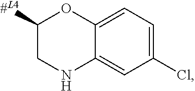

wherein #^{L4} represents the attachment point to L⁴. In some embodiments, R^{E}, independently at each occurrence, is chloro. In some embodiments, E is

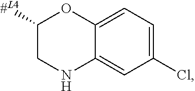

wherein #^{L4} represents the attachment point to L⁴.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof:

### E is

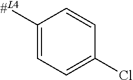

and L⁴ is a bond;

### E is

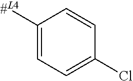

and L⁴ is #^{D}—CH₂—O—$^{E};

### E is

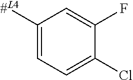

and L⁴ is a bond;

### E is

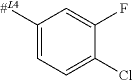

and L⁴ is #^{D}—CH₂—O—$^{E};

### E is

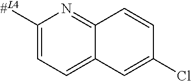

and L⁴ is a bond;

### E is

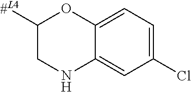

and L⁴ is a bond;

### E is

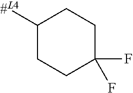

and L⁴ is a bond; or

### E is

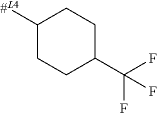

and L⁴ is a bond;
wherein #^{L4} represents the attachment point to L⁴ and #^{D} represents the attachment point to D and $^{E} represents the attachment point to E.
In some embodiments, E is

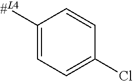

and L⁴ is a bond;

### E is

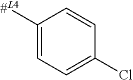

and L⁴ is #^{D}—CH₂—O—$^{E};

### E is

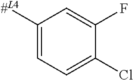

and L⁴ is a bond;

### E is

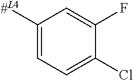

and L⁴ is #^{D}—CH₂—O—$^{E};

### E is

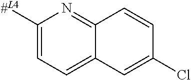

and L⁴ is a bond; or

### E is

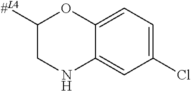

and L⁴ is a bond;
wherein #^{L4} represents the attachment point to L⁴ and #^{D} represents the attachment point to D and $^{E} represents the attachment point to E.
In some embodiments, E is

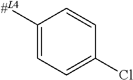

and L⁴ is a bond, wherein #^{L4} represents the attachment point to L⁴ and #^{D} represents the attachment point to D and $^{E} represents the attachment point to E. In some embodiments, E is E is

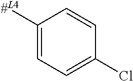

and L⁴ is #^{D}—CH₂—O—$^{E} wherein #^{L4} represents the attachment point to L⁴ and #^{D} represents the attachment point to D and $^{E} represents the attachment point to E. In some embodiments, E is

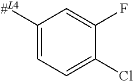

and L⁴ is a bond, wherein #^{L4} represents the attachment point to L⁴ and #^{D} represents the attachment point to D and $^{E} represents the attachment point to E. In some embodiments, E is

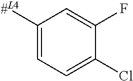

and L⁴ is #^{D}—CH₂—O—$^{E}, wherein #^{L4} represents the attachment point to L⁴ and #^{D} represents the attachment point to D and $^{E} represents the attachment point to E. In some embodiments, E is

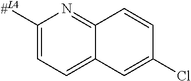

and L⁴ is a bond, wherein #^{L4} represents the attachment point to L⁴ and #^{D} represents the attachment point to D and $^{E} represents the attachment point to E. In some embodiments, E is

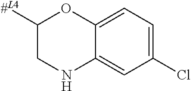

and L⁴ is a bond, wherein #^{L4} represents the attachment point to L⁴ and #^{D} represents the attachment point to D and $^{E} represents the attachment point to E. In some embodiments, E is

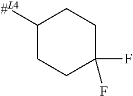

and L⁴ is a bond, wherein #^{L4} represents the attachment point to L⁴ and #^{D} represents the attachment point to D and $^{E} represents the attachment point to E. In some embodiments, E is

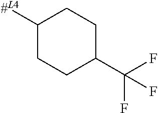

and L⁴ is a bond, wherein #^{L4} represents the attachment point to L⁴ and #^{D} represents the attachment point to D and $^{E} represents the attachment point to E.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L⁴ is a bond or #^{D}—CH₂—O—$^{E}, wherein #^{D} represents the attachment point to D and $^{E} represents the attachment point to E.

In some embodiments of the compounds of formulae (I), (II), (II-i), (II-a), (II-a-i), (III), (III-i), (III-a), (III-a-i), (IV), (IV-i), (V), (V-i), (VI), (VI-i), (VII), (VII-i), (VIII), (VIII-i), (IX), (IX-i), (X), (X-i), (X-a), (X-a-i), (XI), (XI-i), (XI-a), (XI-a-i), (XII), (XII-i), (XII-a), (XII-a-i), (XIII), (XIV), (XIV-i), (XV), (XVI), (XVII), (XVII-i), (XVII-a), (XVII-a-i), (XVIII), (XVIII-i), (XIX), (XIX-i), (XX), and (XX-i), or the salts thereof, L⁴ and E are taken together to form a group selected from the group consisting of C₁-C₆ haloalkyl, (C₁-C₆ alkylene)—O—(C₁-C₆ haloalkyl), —O—(C₁-C₆ alkylene)—O—(C₁-C₆ haloalkyl), and —NH—(C₁-C₆ alkylene)—O—(C₁-C₆ haloalkyl). In some embodiments, L⁴ and E are taken together to form a group selected from the group consisting of

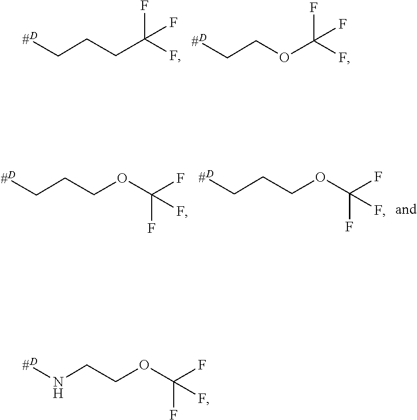

wherein #^{D} represents the attachment point D. In some embodiments, L⁴ and E are taken together to form

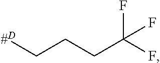

wherein #^{D} represents the attachment point to D. In some embodiments, L⁴ and E are taken together to form

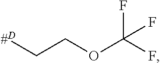

wherein #^{D} represents the attachment point to D. In some embodiments, L⁴ and E are taken together to form

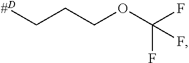

wherein #^{D} represents the attachment point to D. In some embodiments, L⁴ and E are taken together to form

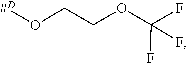

wherein #^{D} represents the attachment point to D. In some embodiments, L⁴ and E are taken together to form

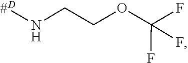

wherein #^{D} represents the attachment point to D.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to A of formula (I) may be combined with every description, variation, embodiment or aspect of R^{A}, L¹, R^{L1}, L², R^{L2}, L³, R^{L3}, B, R^{B}, R^{NB}, R^{BB}, D, R^{D}, L⁴, E, R^{E}, R⁶, R⁷, and R⁸ the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae.

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described and embraced by the invention.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

In some embodiments, provided is compound selected from compounds in Table 1, Table 2, or Table 3, or a stereoisomer, tautomer, solvate, prodrug or salt thereof. Although certain compounds described in Table 1, Table 2, or Table 3 are presented as specific stereoisomers and/or in a non-stereochemical form, it is understood that any or all stereochemical forms, including any enantiomeric or diastereomeric forms, and any tautomers or other forms of any of the compounds of Table 1, Table 2, or Table 3 are herein described.

TABLE 1

| Compound | |
|---|---|
| No. | Structure |
| 1 | 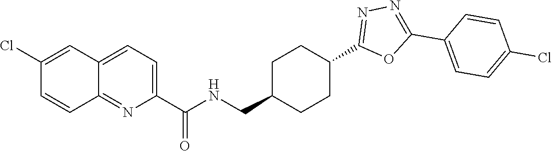 [SMILES: O=C(NC[C@H]1CC[C@H](c2nnc(-c3ccc(Cl)cc3)o2)CC1)c1ccc2cc(Cl)ccc2n1] |
| 2 | 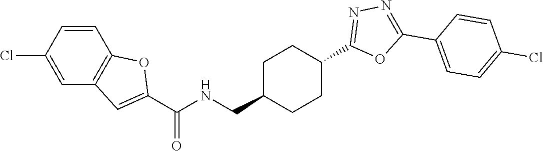 [SMILES: O=C(NC[C@H]1CC[C@H](c2nnc(-c3ccc(Cl)cc3)o2)CC1)c1cc2cc(Cl)ccc2o1] |
| 3 | 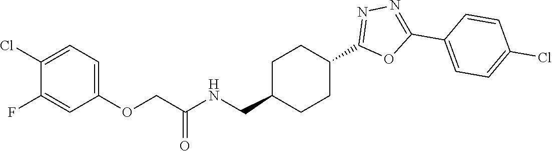 [SMILES: O=C(COc1ccc(Cl)c(F)c1)NC[C@H]1CC[C@H](c2nnc(-c3ccc(Cl)cc3)o2)CC1] |
| 4 | 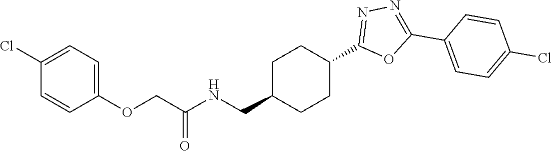 [SMILES: O=C(COc1ccc(Cl)cc1)NC[C@H]1CC[C@H](c2nnc(-c3ccc(Cl)cc3)o2)CC1] |
| 5 | 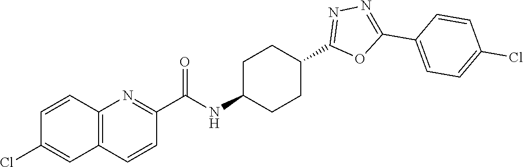 [SMILES: O=C(N[C@H]1CC[C@H](c2nnc(-c3ccc(Cl)cc3)o2)CC1)c1ccc2cc(Cl)ccc2n1] |
| 6 | 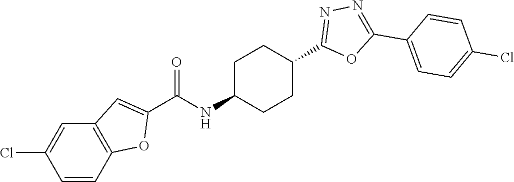 [SMILES: O=C(N[C@H]1CC[C@H](c2nnc(-c3ccc(Cl)cc3)o2)CC1)c1cc2cc(Cl)ccc2o1] |
| 7 | 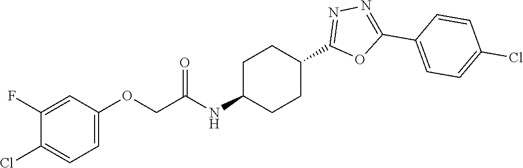 [SMILES: O=C(COc1ccc(Cl)c(F)c1)N[C@H]1CC[C@H](c2nnc(-c3ccc(Cl)cc3)o2)CC1] |
| 8 | 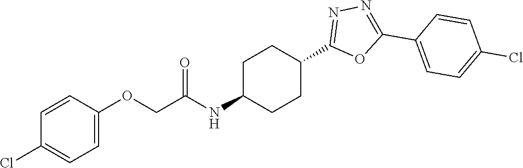 [SMILES: O=C(COc1ccc(Cl)cc1)N[C@H]1CC[C@H](c2nnc(-c3ccc(Cl)cc3)o2)CC1] |
| 9 | 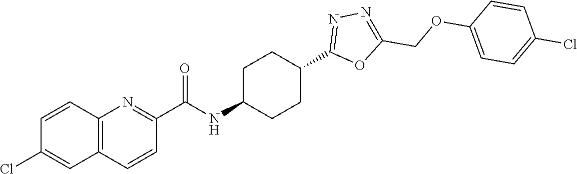 [SMILES: O=C(N[C@H]1CC[C@H](c2nnc(COc3ccc(Cl)cc3)o2)CC1)c1ccc2cc(Cl)ccc2n1] |
| 10 | 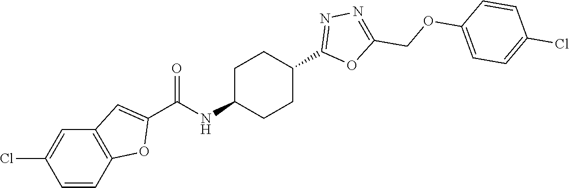 [SMILES: O=C(N[C@H]1CC[C@H](c2nnc(COc3ccc(Cl)cc3)o2)CC1)c1cc2cc(Cl)ccc2o1] |
| 11 | 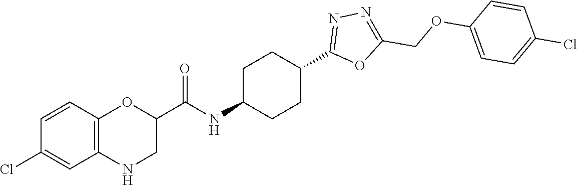 [SMILES: O=C(N[C@H]1CC[C@H](c2nnc(COc3ccc(Cl)cc3)o2)CC1)C1CNc2cc(Cl)ccc2O1] |
| 12 | 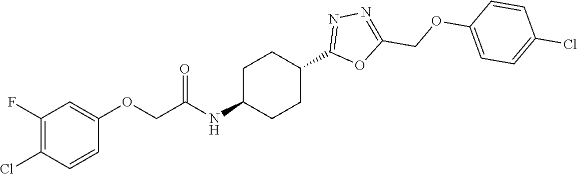 [SMILES: O=C(COc1ccc(Cl)c(F)c1)N[C@H]1CC[C@H](c2nnc(COc3ccc(Cl)cc3)o2)CC1] |
| 13 | 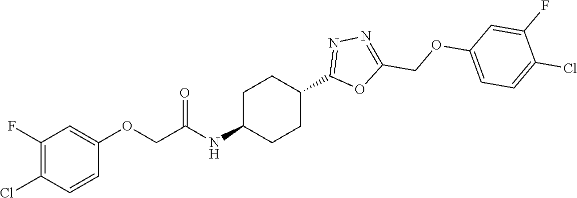 [SMILES: O=C(COc1ccc(Cl)c(F)c1)N[C@H]1CC[C@H](c2nnc(COc3ccc(Cl)c(F)c3)o2)CC1] |
| 14 | 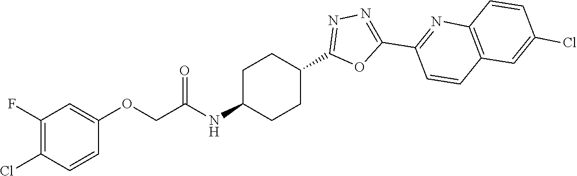 [SMILES: O=C(COc1ccc(Cl)c(F)c1)N[C@H]1CC[C@H](c2nnc(-c3ccc4cc(Cl)ccc4n3)o2)CC1] |
| 15 | 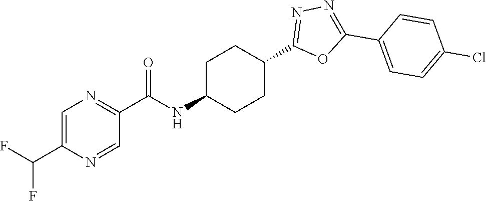 [SMILES: O=C(N[C@H]1CC[C@H](c2nnc(-c3ccc(Cl)cc3)o2)CC1)c1cnc(C(F)F)cn1] |
| 16 | 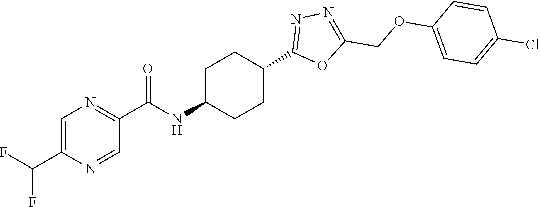 [SMILES: O=C(N[C@H]1CC[C@H](c2nnc(COc3ccc(Cl)cc3)o2)CC1)c1cnc(C(F)F)cn1] |
| 17 | 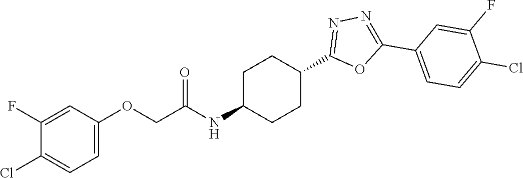 [SMILES: O=C(COc1ccc(Cl)c(F)c1)N[C@H]1CC[C@H](c2nnc(-c3ccc(Cl)c(F)c3)o2)CC1] |
| 18 | 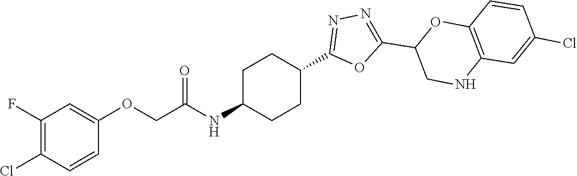 [SMILES: O=C(COc1ccc(Cl)c(F)c1)N[C@H]1CC[C@H](c2nnc(C3CNc4cc(Cl)ccc4O3)o2)CC1] |
| 19 | 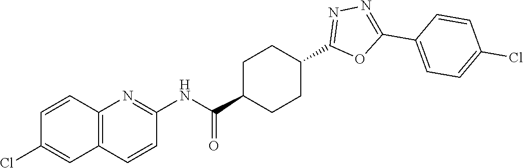 [SMILES: O=C(Nc1ccc2cc(Cl)ccc2n1)[C@H]1CC[C@H](c2nnc(-c3ccc(Cl)cc3)o2)CC1] |
| 20 | 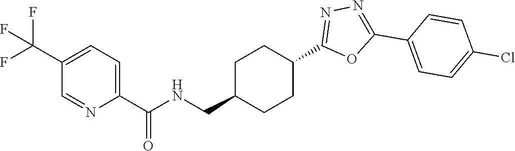 [SMILES: O=C(NC[C@H]1CC[C@H](c2nnc(-c3ccc(Cl)cc3)o2)CC1)c1ccc(C(F)(F)F)cn1] |
| 21 | 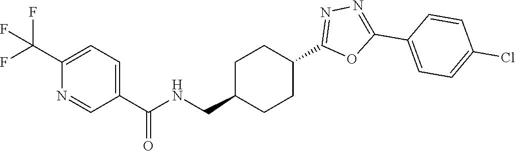 [SMILES: O=C(NC[C@H]1CC[C@H](c2nnc(-c3ccc(Cl)cc3)o2)CC1)c1ccc(C(F)(F)F)nc1] |
| 22 | 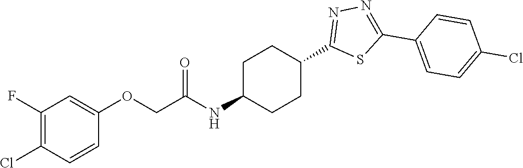 [SMILES: O=C(COc1ccc(Cl)c(F)c1)N[C@H]1CC[C@H](c2nnc(-c3ccc(Cl)cc3)s2)CC1] |
| 23 | 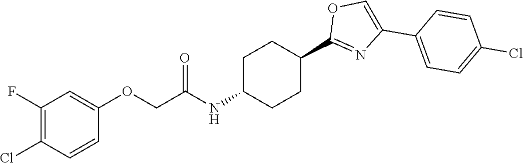 [SMILES: O=C(COc1ccc(Cl)c(F)c1)N[C@H]1CC[C@H](c2nc(-c3ccc(Cl)cc3)co2)CC1] |
| 24 | 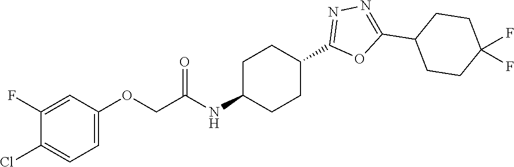 [SMILES: O=C(COc1ccc(Cl)c(F)c1)N[C@H]1CC[C@H](c2nnc(C3CCC(F)(F)CC3)o2)CC1] |
| 25 | 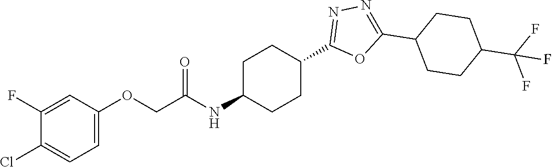 [SMILES: O=C(COc1ccc(Cl)c(F)c1)N[C@H]1CC[C@H](c2nnc(C3CCC(C(F)(F)F)CC3)o2)CC1] |
| 26 | 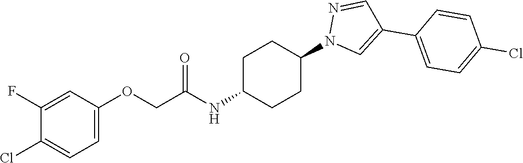 [SMILES: O=C(COc1ccc(Cl)c(F)c1)N[C@H]1CC[C@H](n2cc(-c3ccc(Cl)cc3)cn2)CC1] |
| 27 | 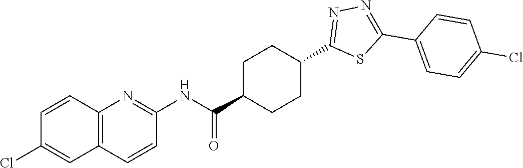 [SMILES: O=C(Nc1ccc2cc(Cl)ccc2n1)[C@H]1CC[C@H](c2nnc(-c3ccc(Cl)cc3)s2)CC1] |

TABLE 2

| Compound | |
|---|---|
| No. | Structure |
| 28 | 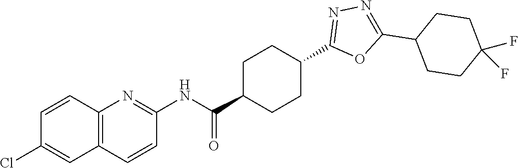 [SMILES: O=C(Nc1ccc2cc(Cl)ccc2n1)[C@H]1CC[C@H](c2nnc(C3CCC(F)(F)CC3)o2)CC1] |
| 29 | 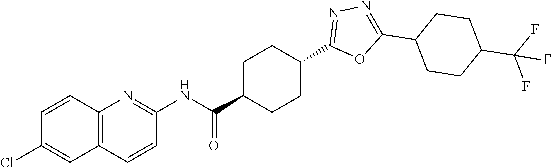 [SMILES: O=C(Nc1ccc2cc(Cl)ccc2n1)[C@H]1CC[C@H](c2nnc(C3CCC(C(F)(F)F)CC3)o2)CC1] |
| 30 | 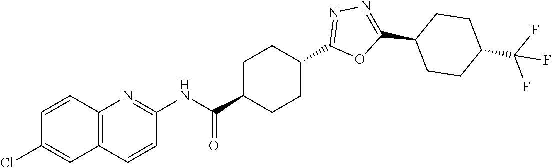 [SMILES: O=C(Nc1ccc2cc(Cl)ccc2n1)[C@H]1CC[C@H](c2nnc([C@H]3CC[C@H](C(F)(F)F)CC3)o2)CC1] |
| 31 | 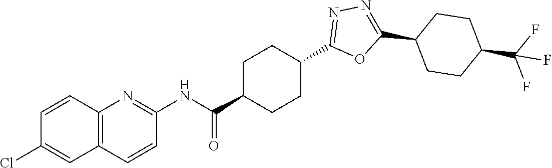 [SMILES: O=C(Nc1ccc2cc(Cl)ccc2n1)[C@H]1CC[C@H](c2nnc([C@H]3CC[C@@H](C(F)(F)F)CC3)o2)CC1] |
| 32 | 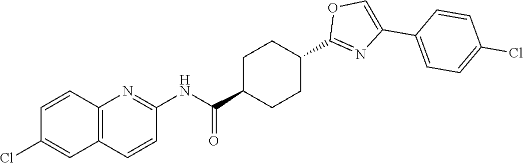 [SMILES: O=C(Nc1ccc2cc(Cl)ccc2n1)[C@H]1CC[C@H](c2nc(-c3ccc(Cl)cc3)co2)CC1] |
| 33 | 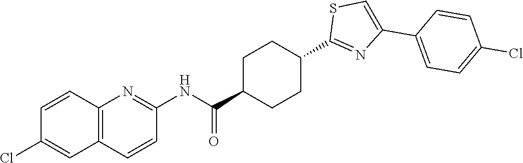 [SMILES: O=C(Nc1ccc2cc(Cl)ccc2n1)[C@H]1CC[C@H](c2nc(-c3ccc(Cl)cc3)cs2)CC1] |
| 34 | 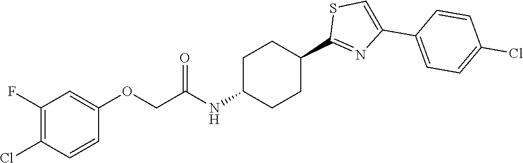 [SMILES: O=C(COc1ccc(Cl)c(F)c1)N[C@H]1CC[C@H](c2nc(-c3ccc(Cl)cc3)cs2)CC1] |
| 35 | 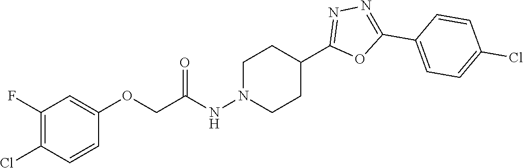 [SMILES: O=C(COc1ccc(Cl)c(F)c1)NN1CCC(c2nnc(-c3ccc(Cl)cc3)o2)CC1] |
| 36 | 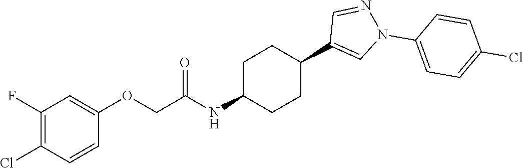 [SMILES: O=C(COc1ccc(Cl)c(F)c1)N[C@H]1CC[C@@H](c2cnn(-c3ccc(Cl)cc3)c2)CC1] |
| 37 | 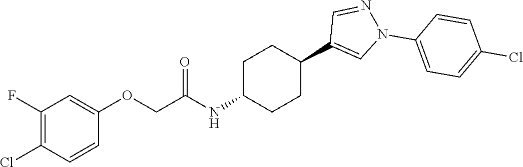 [SMILES: O=C(COc1ccc(Cl)c(F)c1)N[C@H]1CC[C@H](c2cnn(-c3ccc(Cl)cc3)c2)CC1] |
| 38 | 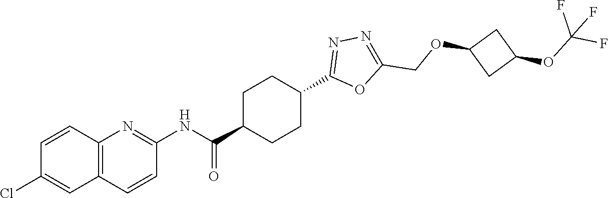 [SMILES: O=C(Nc1ccc2cc(Cl)ccc2n1)[C@H]1CC[C@H](c2nnc(CO[C@H]3C[C@@H](OC(F)(F)F)C3)o2)CC1] |
| 39 | 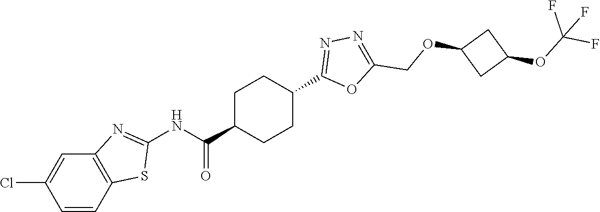 [SMILES: O=C(Nc1nc2cc(Cl)ccc2s1)[C@H]1CC[C@H](c2nnc(CO[C@H]3C[C@@H](OC(F)(F)F)C3)o2)CC1] |
| 40 | 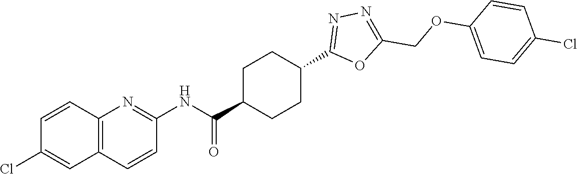 [SMILES: O=C(Nc1ccc2cc(Cl)ccc2n1)[C@H]1CC[C@H](c2nnc(COc3ccc(Cl)cc3)o2)CC1] |
| 41 | 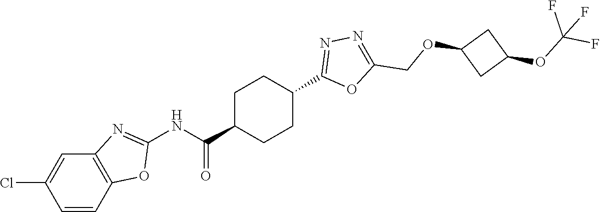 [SMILES: O=C(Nc1nc2cc(Cl)ccc2o1)[C@H]1CC[C@H](c2nnc(CO[C@H]3C[C@@H](OC(F)(F)F)C3)o2)CC1] |
| 42 | 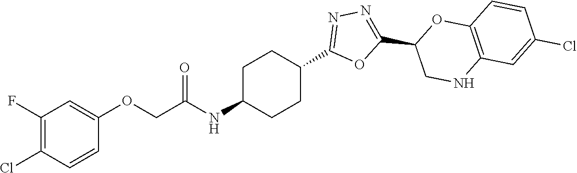 [SMILES: O=C(COc1ccc(Cl)c(F)c1)N[C@H]1CC[C@H](c2nnc([C@@H]3CNc4cc(Cl)ccc4O3)o2)CC1] |
| 43 | 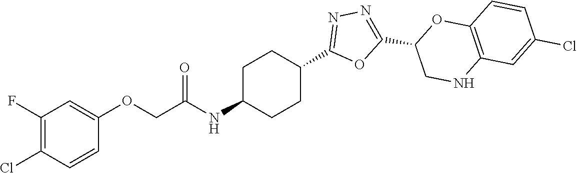 [SMILES: O=C(COc1ccc(Cl)c(F)c1)N[C@H]1CC[C@H](c2nnc([C@H]3CNc4cc(Cl)ccc4O3)o2)CC1] |
| 44 | 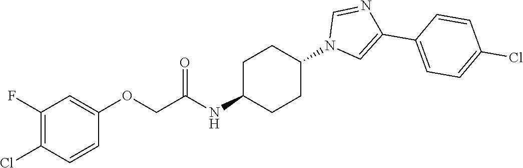 [SMILES: O=C(COc1ccc(Cl)c(F)c1)N[C@H]1CC[C@H](n2cnc(-c3ccc(Cl)cc3)c2)CC1] |
| 45 | 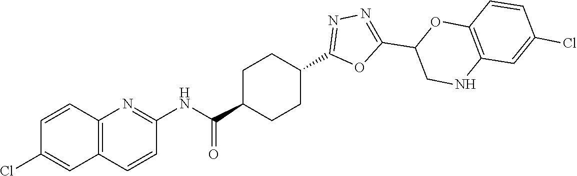 [SMILES: O=C(Nc1ccc2cc(Cl)ccc2n1)[C@H]1CC[C@H](c2nnc(C3CNc4cc(Cl)ccc4O3)o2)CC1] |
| 46 | 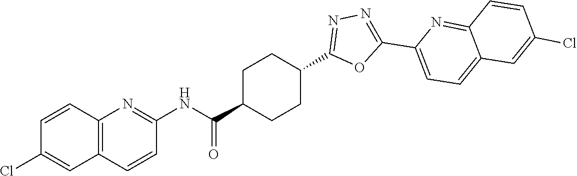 [SMILES: O=C(Nc1ccc2cc(Cl)ccc2n1)[C@H]1CC[C@H](c2nnc(-c3ccc4cc(Cl)ccc4n3)o2)CC1] |
| 47 | 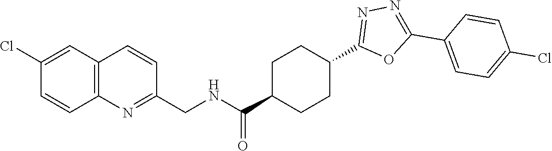 [SMILES: O=C(NCc1ccc2cc(Cl)ccc2n1)[C@H]1CC[C@H](c2nnc(-c3ccc(Cl)cc3)o2)CC1] |
| 48 | 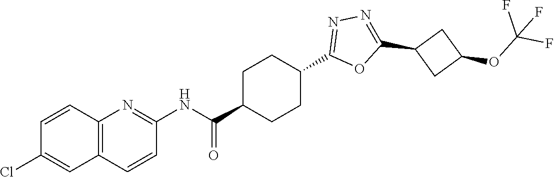 [SMILES: O=C(Nc1ccc2cc(Cl)ccc2n1)[C@H]1CC[C@H](c2nnc([C@H]3C[C@@H](OC(F)(F)F)C3)o2)CC1] |
| 49 | 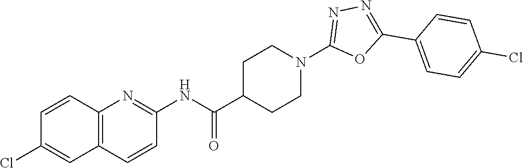 [SMILES: O=C(Nc1ccc2cc(Cl)ccc2n1)C1CCN(c2nnc(-c3ccc(Cl)cc3)o2)CC1] |
| 50 | 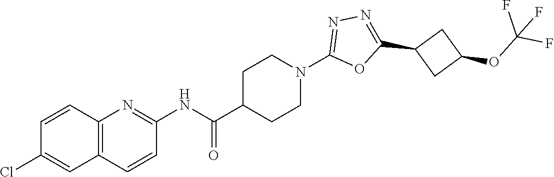 [SMILES: O=C(Nc1ccc2cc(Cl)ccc2n1)C1CCN(c2nnc([C@H]3C[C@@H](OC(F)(F)F)C3)o2)CC1] |
| 51 | 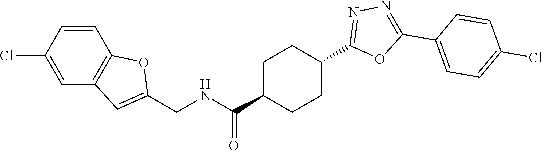 [SMILES: O=C(NCc1cc2cc(Cl)ccc2o1)[C@H]1CC[C@H](c2nnc(-c3ccc(Cl)cc3)o2)CC1] |
| 52 | 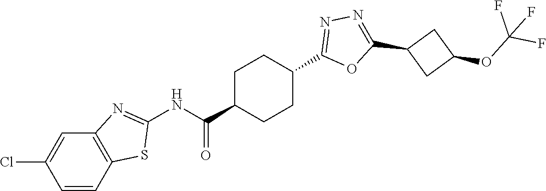 [SMILES: O=C(Nc1nc2cc(Cl)ccc2s1)[C@H]1CC[C@H](c2nnc([C@H]3C[C@@H](OC(F)(F)F)C3)o2)CC1] |
| 53 | 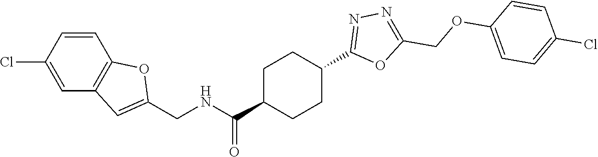 [SMILES: O=C(NCc1cc2cc(Cl)ccc2o1)[C@H]1CC[C@H](c2nnc(COc3ccc(Cl)cc3)o2)CC1] |
| 54 | 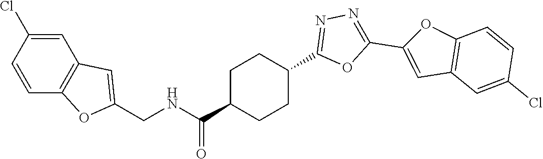 [SMILES: O=C(NCc1cc2cc(Cl)ccc2o1)[C@H]1CC[C@H](c2nnc(-c3cc4cc(Cl)ccc4o3)o2)CC1] |

TABLE 3

| Compound | |
|---|---|
| No. | Structure |
| 55 | 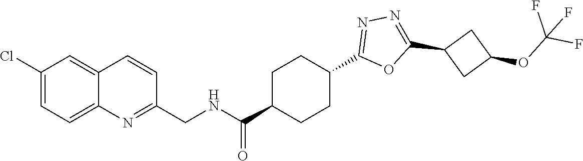 [SMILES: O=C(NCc1ccc2cc(Cl)ccc2n1)[C@H]1CC[C@H](c2nnc([C@H]3C[C@@H](OC(F)(F)F)C3)o2)CC1] |
| 56 | 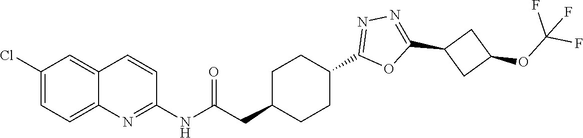 [SMILES: O=C(C[C@H]1CC[C@H](c2nnc([C@H]3C[C@@H](OC(F)(F)F)C3)o2)CC1)Nc1ccc2cc(Cl)ccc2n1] |
| 57 | 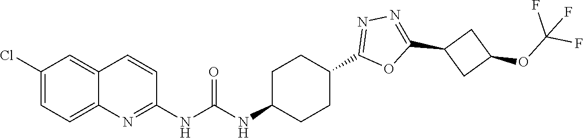 [SMILES: O=C(Nc1ccc2cc(Cl)ccc2n1)N[C@H]1CC[C@H](c2nnc([C@H]3C[C@@H](OC(F)(F)F)C3)o2)CC1] |
| 58 | 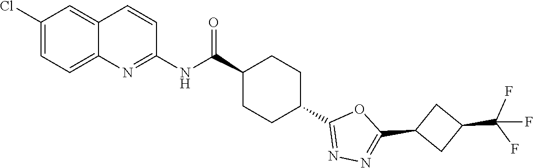 [SMILES: O=C(Nc1ccc2cc(Cl)ccc2n1)[C@H]1CC[C@H](c2nnc([C@H]3C[C@@H](C(F)(F)F)C3)o2)CC1] |
| 59 | 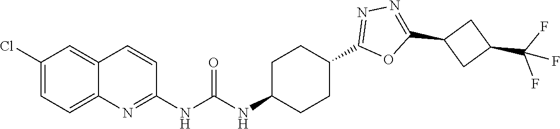 [SMILES: O=C(Nc1ccc2cc(Cl)ccc2n1)N[C@H]1CC[C@H](c2nnc([C@H]3C[C@@H](C(F)(F)F)C3)o2)CC1] |
| 60 | 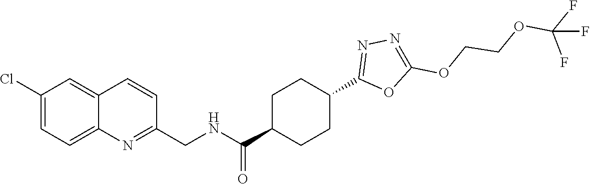 [SMILES: O=C(NCc1ccc2cc(Cl)ccc2n1)[C@H]1CC[C@H](c2nnc(OCCOC(F)(F)F)o2)CC1] |
| 61 | 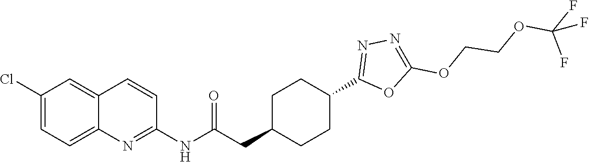 [SMILES: O=C(C[C@H]1CC[C@H](c2nnc(OCCOC(F)(F)F)o2)CC1)Nc1ccc2cc(Cl)ccc2n1] |
| 62 | 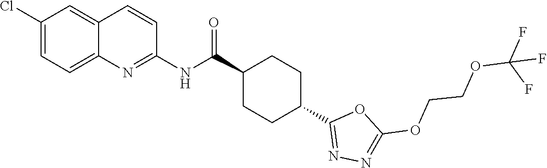 [SMILES: O=C(Nc1ccc2cc(Cl)ccc2n1)[C@H]1CC[C@H](c2nnc(OCCOC(F)(F)F)o2)CC1] |
| 63 | 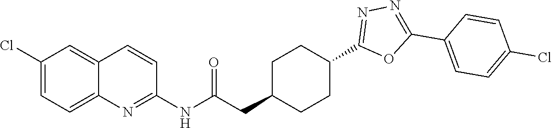 [SMILES: O=C(C[C@H]1CC[C@H](c2nnc(-c3ccc(Cl)cc3)o2)CC1)Nc1ccc2cc(Cl)ccc2n1] |
| 64 | 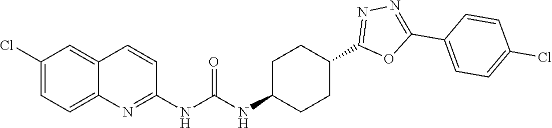 [SMILES: O=C(Nc1ccc2cc(Cl)ccc2n1)N[C@H]1CC[C@H](c2nnc(-c3ccc(Cl)cc3)o2)CC1] |
| 65 | 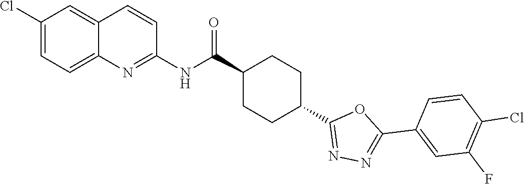 [SMILES: O=C(Nc1ccc2cc(Cl)ccc2n1)[C@H]1CC[C@H](c2nnc(-c3ccc(Cl)c(F)c3)o2)CC1] |
| 66 | 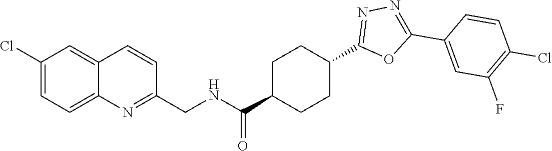 [SMILES: O=C(NCc1ccc2cc(Cl)ccc2n1)[C@H]1CC[C@H](c2nnc(-c3ccc(Cl)c(F)c3)o2)CC1] |
| 67 | 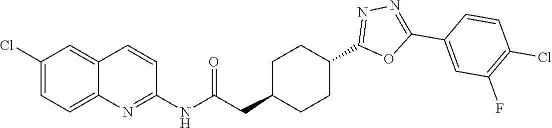 [SMILES: O=C(C[C@H]1CC[C@H](c2nnc(-c3ccc(Cl)c(F)c3)o2)CC1)Nc1ccc2cc(Cl)ccc2n1] |
| 68 | 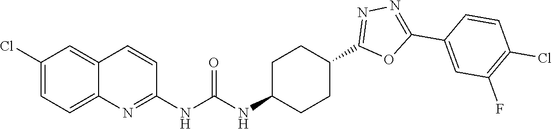 [SMILES: O=C(Nc1ccc2cc(Cl)ccc2n1)N[C@H]1CC[C@H](c2nnc(-c3ccc(Cl)c(F)c3)o2)CC1] |
| 69 | 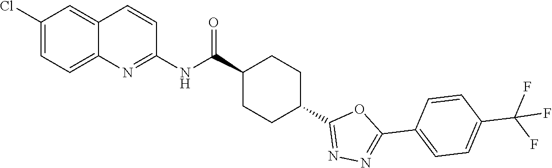 [SMILES: O=C(Nc1ccc2cc(Cl)ccc2n1)[C@H]1CC[C@H](c2nnc(-c3ccc(C(F)(F)F)cc3)o2)CC1] |
| 70 | 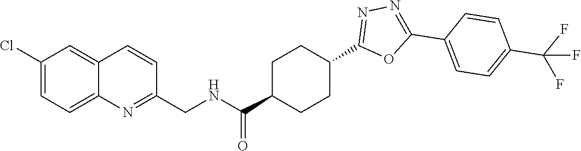 [SMILES: O=C(NCc1ccc2cc(Cl)ccc2n1)[C@H]1CC[C@H](c2nnc(-c3ccc(C(F)(F)F)cc3)o2)CC1] |
| 71 | 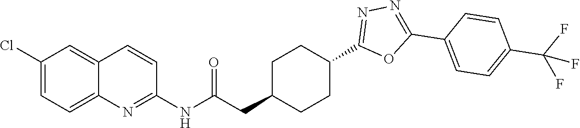 [SMILES: O=C(C[C@H]1CC[C@H](c2nnc(-c3ccc(C(F)(F)F)cc3)o2)CC1)Nc1ccc2cc(Cl)ccc2n1] |
| 72 | 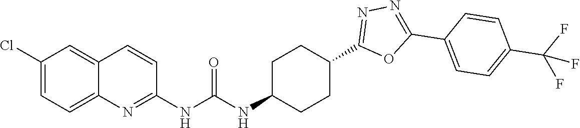 [SMILES: O=C(Nc1ccc2cc(Cl)ccc2n1)N[C@H]1CC[C@H](c2nnc(-c3ccc(C(F)(F)F)cc3)o2)CC1] |
| 73 | 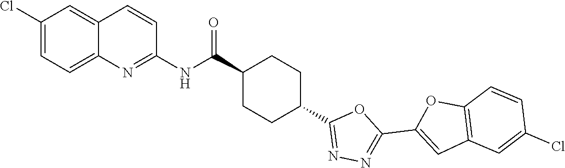 [SMILES: O=C(Nc1ccc2cc(Cl)ccc2n1)[C@H]1CC[C@H](c2nnc(-c3cc4cc(Cl)ccc4o3)o2)CC1] |
| 74 | 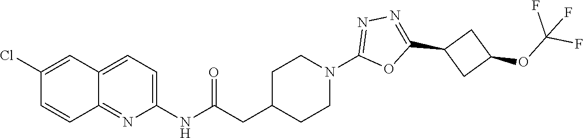 [SMILES: O=C(CC1CCN(c2nnc([C@H]3C[C@@H](OC(F)(F)F)C3)o2)CC1)Nc1ccc2cc(Cl)ccc2n1] |
| 75 | 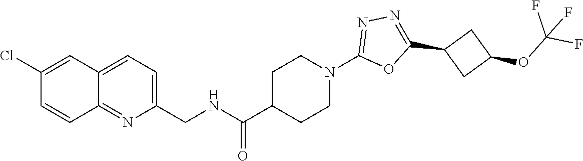 [SMILES: O=C(NCc1ccc2cc(Cl)ccc2n1)C1CCN(c2nnc([C@H]3C[C@@H](OC(F)(F)F)C3)o2)CC1] |
| 76 | 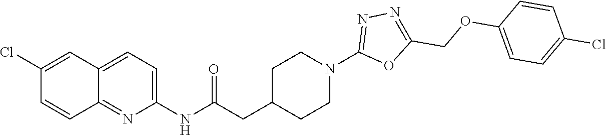 [SMILES: O=C(CC1CCN(c2nnc(COc3ccc(Cl)cc3)o2)CC1)Nc1ccc2cc(Cl)ccc2n1] |
| 77 | 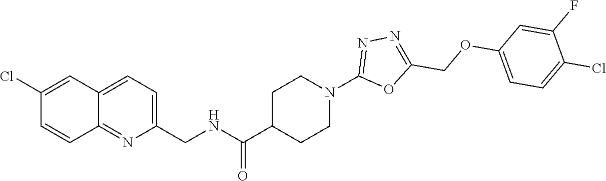 [SMILES: O=C(NCc1ccc2cc(Cl)ccc2n1)C1CCN(c2nnc(COc3ccc(Cl)c(F)c3)o2)CC1] |
| 78 | 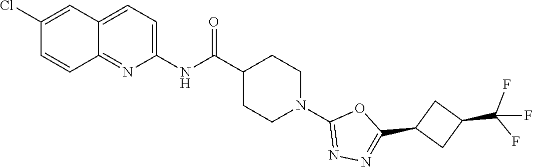 [SMILES: O=C(Nc1ccc2cc(Cl)ccc2n1)C1CCN(c2nnc([C@H]3C[C@@H](C(F)(F)F)C3)o2)CC1] |
| 79 | 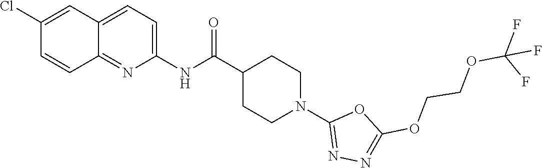 [SMILES: O=C(Nc1ccc2cc(Cl)ccc2n1)C1CCN(c2nnc(OCCOC(F)(F)F)o2)CC1] |
| 80 | 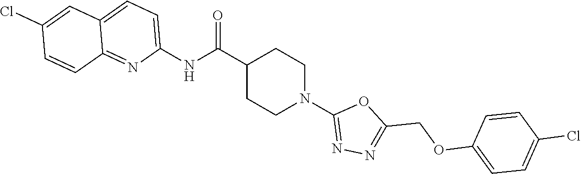 [SMILES: O=C(Nc1ccc2cc(Cl)ccc2n1)C1CCN(c2nnc(COc3ccc(Cl)cc3)o2)CC1] |
| 81 | 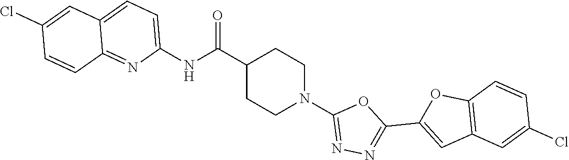 [SMILES: O=C(Nc1ccc2cc(Cl)ccc2n1)C1CCN(c2nnc(-c3cc4cc(Cl)ccc4o3)o2)CC1] |
| 82 | 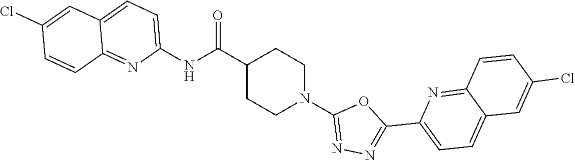 [SMILES: O=C(Nc1ccc2cc(Cl)ccc2n1)C1CCN(c2nnc(-c3ccc4cc(Cl)ccc4n3)o2)CC1] |
| 83 | 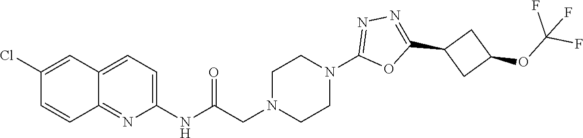 [SMILES: O=C(CN1CCN(c2nnc([C@H]3C[C@@H](OC(F)(F)F)C3)o2)CC1)Nc1ccc2cc(Cl)ccc2n1] |
| 84 | 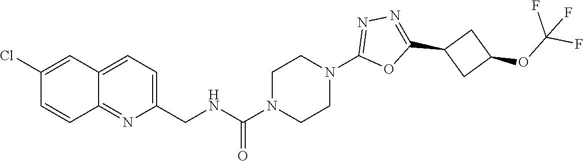 [SMILES: O=C(NCc1ccc2cc(Cl)ccc2n1)N1CCN(c2nnc([C@H]3C[C@@H](OC(F)(F)F)C3)o2)CC1] |
| 85 | 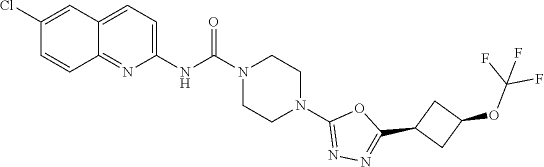 [SMILES: O=C(Nc1ccc2cc(Cl)ccc2n1)N1CCN(c2nnc([C@H]3C[C@@H](OC(F)(F)F)C3)o2)CC1] |
| 86 | 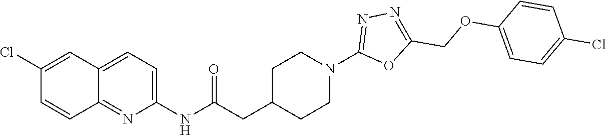 [SMILES: O=C(CC1CCN(c2nnc(COc3ccc(Cl)cc3)o2)CC1)Nc1ccc2cc(Cl)ccc2n1] |
| 87 | 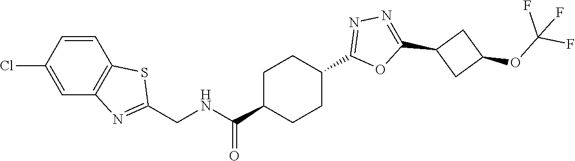 [SMILES: O=C(NCc1nc2cc(Cl)ccc2s1)[C@H]1CC[C@H](c2nnc([C@H]3C[C@@H](OC(F)(F)F)C3)o2)CC1] |
| 88 | 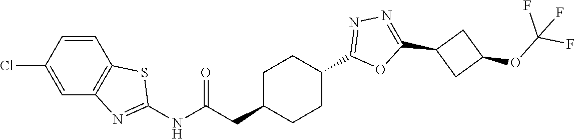 [SMILES: O=C(C[C@H]1CC[C@H](c2nnc([C@H]3C[C@@H](OC(F)(F)F)C3)o2)CC1)Nc1nc2cc(Cl)ccc2s1] |
| 89 | 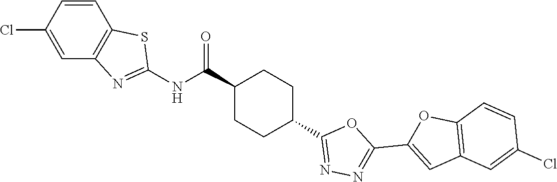 [SMILES: O=C(Nc1nc2cc(Cl)ccc2s1)[C@H]1CC[C@H](c2nnc(-c3cc4cc(Cl)ccc4o3)o2)CC1] |
| 90 | 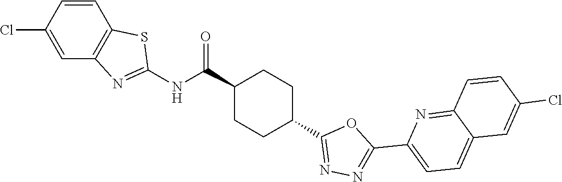 [SMILES: O=C(Nc1nc2cc(Cl)ccc2s1)[C@H]1CC[C@H](c2nnc(-c3ccc4cc(Cl)ccc4n3)o2)CC1] |
| 91 | 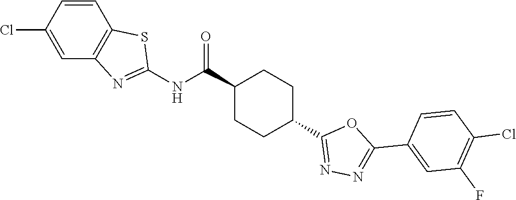 [SMILES: O=C(Nc1nc2cc(Cl)ccc2s1)[C@H]1CC[C@H](c2nnc(-c3ccc(Cl)c(F)c3)o2)CC1] |
| 92 | 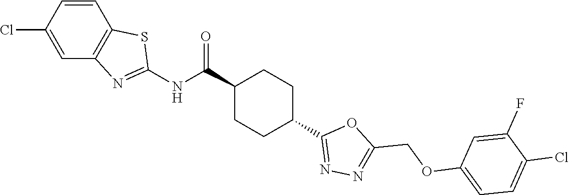 [SMILES: O=C(Nc1nc2cc(Cl)ccc2s1)[C@H]1CC[C@H](c2nnc(COc3ccc(Cl)c(F)c3)o2)CC1] |
| 93 | 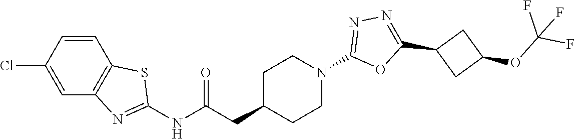 |
| 94 | 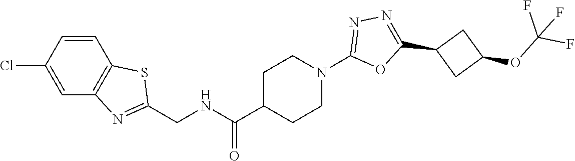 [SMILES: O=C(NCc1nc2cc(Cl)ccc2s1)C1CCN(c2nnc([C@H]3C[C@@H](OC(F)(F)F)C3)o2)CC1] |
| 95 | 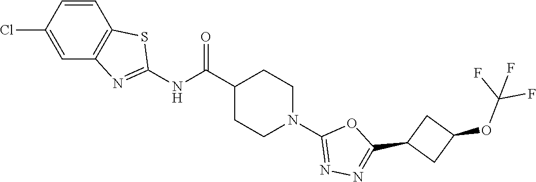 [SMILES: O=C(Nc1nc2cc(Cl)ccc2s1)C1CCN(c2nnc([C@H]3C[C@@H](OC(F)(F)F)C3)o2)CC1] |
| 96 | 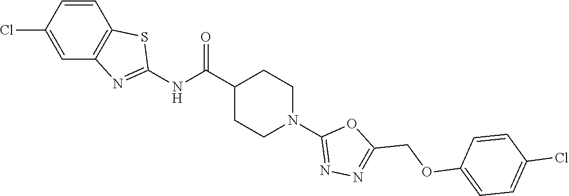 [SMILES: O=C(Nc1nc2cc(Cl)ccc2s1)C1CCN(c2nnc(COc3ccc(Cl)cc3)o2)CC1] |
| 97 | 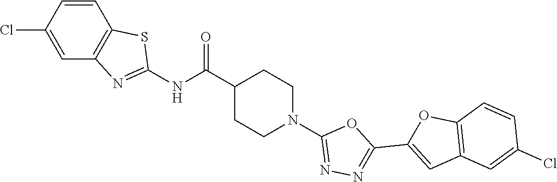 [SMILES: O=C(Nc1nc2cc(Cl)ccc2s1)C1CCN(c2nnc(-c3cc4cc(Cl)ccc4o3)o2)CC1] |
| 98 | 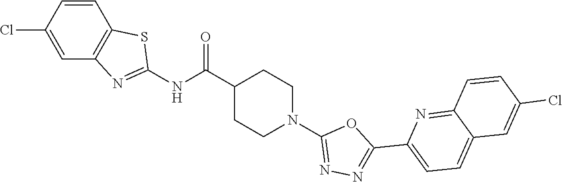 [SMILES: O=C(Nc1nc2cc(Cl)ccc2s1)C1CCN(c2nnc(-c3ccc4cc(Cl)ccc4n3)o2)CC1] |
| 99 | 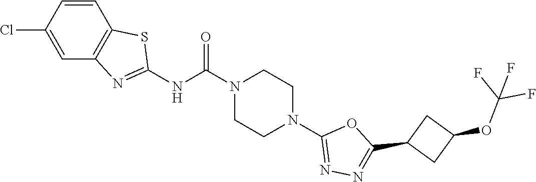 [SMILES: O=C(Nc1nc2cc(Cl)ccc2s1)N1CCN(c2nnc([C@H]3C[C@@H](OC(F)(F)F)C3)o2)CC1] |
| 100 | 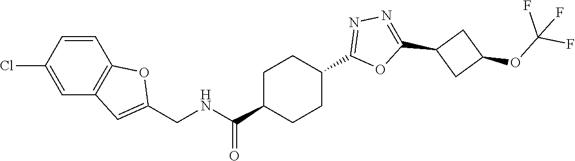 [SMILES: O=C(NCc1cc2cc(Cl)ccc2o1)[C@H]1CC[C@H](c2nnc([C@H]3C[C@@H](OC(F)(F)F)C3)o2)CC1] |
| 101 | 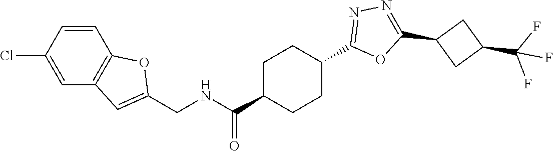 [SMILES: O=C(NCc1cc2cc(Cl)ccc2o1)[C@H]1CC[C@H](c2nnc([C@H]3C[C@@H](C(F)(F)F)C3)o2)CC1] |
| 102 | 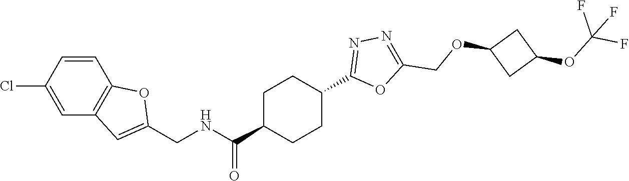 [SMILES: O=C(NCc1cc2cc(Cl)ccc2o1)[C@H]1CC[C@H](c2nnc(CO[C@H]3C[C@@H](OC(F)(F)F)C3)o2)CC1] |
| 103 | 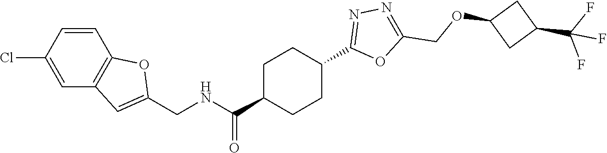 [SMILES: O=C(NCc1cc2cc(Cl)ccc2o1)[C@H]1CC[C@H](c2nnc(CO[C@H]3C[C@@H](C(F)(F)F)C3)o2)CC1] |
| 104 | 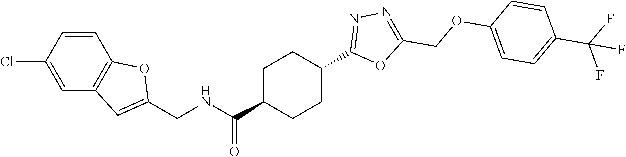 [SMILES: O=C(NCc1cc2cc(Cl)ccc2o1)[C@H]1CC[C@H](c2nnc(COc3ccc(C(F)(F)F)cc3)o2)CC1] |
| 105 | 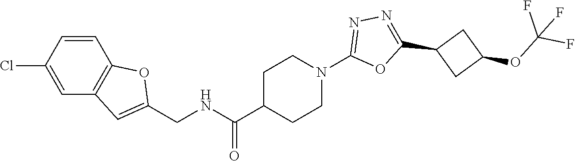 [SMILES: O=C(NCc1cc2cc(Cl)ccc2o1)C1CCN(c2nnc([C@H]3C[C@@H](OC(F)(F)F)C3)o2)CC1] |
| 106 | 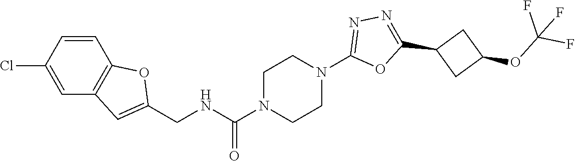 [SMILES: O=C(NCc1cc2cc(Cl)ccc2o1)N1CCN(c2nnc([C@H]3C[C@@H](OC(F)(F)F)C3)o2)CC1] |
| 107 | 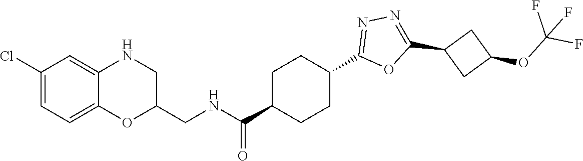 [SMILES: O=C(NCC1CNc2cc(Cl)ccc2O1)[C@H]1CC[C@H](c2nnc([C@H]3C[C@@H](OC(F)(F)F)C3)o2)CC1] |
| 108 | 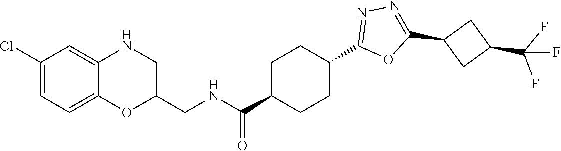 [SMILES: O=C(NCC1CNc2cc(Cl)ccc2O1)[C@H]1CC[C@H](c2nnc([C@H]3C[C@@H](C(F)(F)F)C3)o2)CC1] |
| 109 | 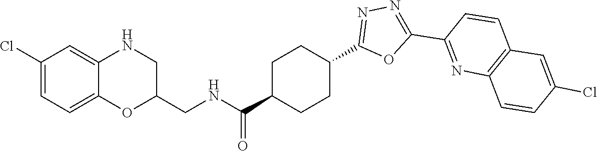 [SMILES: O=C(NCC1CNc2cc(Cl)ccc2O1)[C@H]1CC[C@H](c2nnc(-c3ccc4cc(Cl)ccc4n3)o2)CC1] |
| 110 | 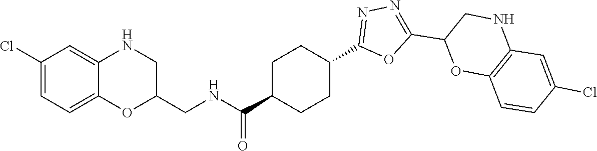 [SMILES: O=C(NCC1CNc2cc(Cl)ccc2O1)[C@H]1CC[C@H](c2nnc(C3CNc4cc(Cl)ccc4O3)o2)CC1] |
| 111 | 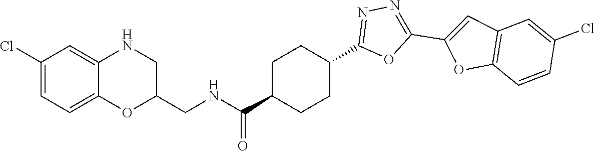 [SMILES: O=C(NCC1CNc2cc(Cl)ccc2O1)[C@H]1CC[C@H](c2nnc(-c3cc4cc(Cl)ccc4o3)o2)CC1] |
| 112 | 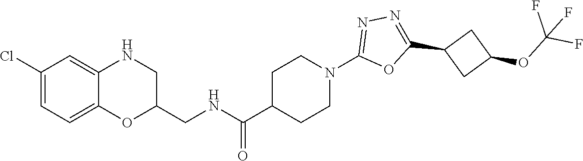 [SMILES: O=C(NCC1CNc2cc(Cl)ccc2O1)C1CCN(c2nnc([C@H]3C[C@@H](OC(F)(F)F)C3)o2)CC1] |
| 113 | 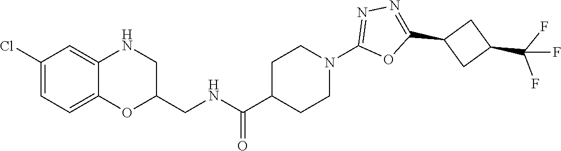 [SMILES: O=C(NCC1CNc2cc(Cl)ccc2O1)C1CCN(c2nnc([C@H]3C[C@@H](C(F)(F)F)C3)o2)CC1] |
| 114 | 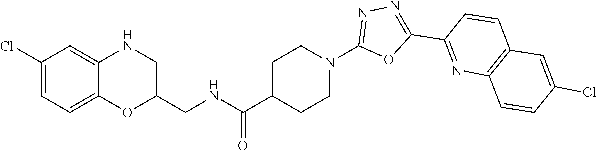 [SMILES: O=C(NCC1CNc2cc(Cl)ccc2O1)C1CCN(c2nnc(-c3ccc4cc(Cl)ccc4n3)o2)CC1] |
| 115 | 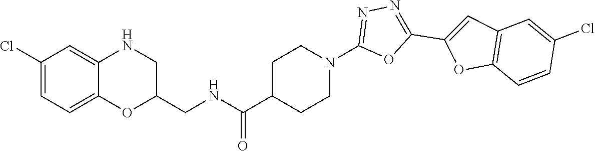 [SMILES: O=C(NCC1CNc2cc(Cl)ccc2O1)C1CCN(c2nnc(-c3cc4cc(Cl)ccc4o3)o2)CC1] |
| 116 | 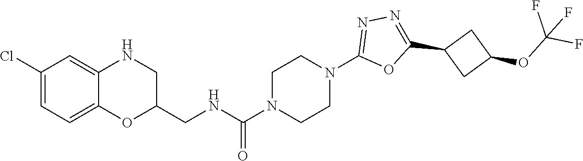 [SMILES: O=C(NCC1CNc2cc(Cl)ccc2O1)N1CCN(c2nnc([C@H]3C[C@@H](OC(F)(F)F)C3)o2)CC1] |
| 117 | 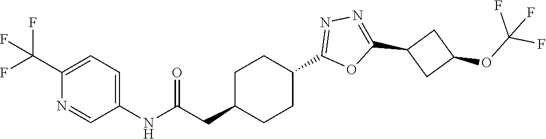 [SMILES: O=C(C[C@H]1CC[C@H](c2nnc([C@H]3C[C@@H](OC(F)(F)F)C3)o2)CC1)Nc1ccc(C(F)(F)F)nc1] |
| 118 | 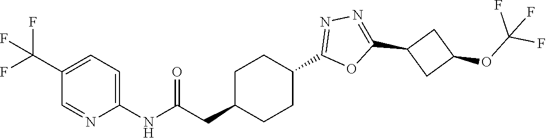 [SMILES: O=C(C[C@H]1CC[C@H](c2nnc([C@H]3C[C@@H](OC(F)(F)F)C3)o2)CC1)Nc1ccc(C(F)(F)F)cn1] |
| 119 | 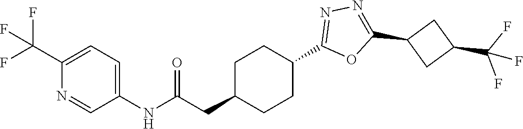 [SMILES: O=C(C[C@H]1CC[C@H](c2nnc([C@H]3C[C@@H](C(F)(F)F)C3)o2)CC1)Nc1ccc(C(F)(F)F)nc1] |
| 120 | 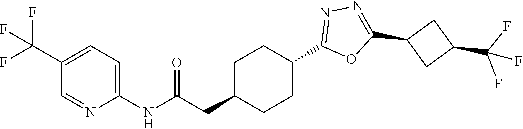 [SMILES: O=C(C[C@H]1CC[C@H](c2nnc([C@H]3C[C@@H](C(F)(F)F)C3)o2)CC1)Nc1ccc(C(F)(F)F)cn1] |
| 121 | 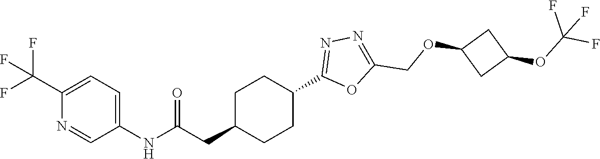 [SMILES: O=C(C[C@H]1CC[C@H](c2nnc(CO[C@H]3C[C@@H](OC(F)(F)F)C3)o2)CC1)Nc1ccc(C(F)(F)F)nc1] |
| 122 | 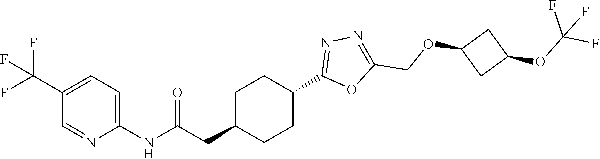 [SMILES: O=C(C[C@H]1CC[C@H](c2nnc(CO[C@H]3C[C@@H](OC(F)(F)F)C3)o2)CC1)Nc1ccc(C(F)(F)F)cn1] |
| 123 | 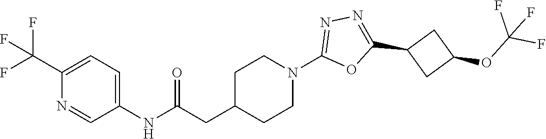 [SMILES: O=C(CC1CCN(c2nnc([C@H]3C[C@@H](OC(F)(F)F)C3)o2)CC1)Nc1ccc(C(F)(F)F)nc1] |
| 124 | 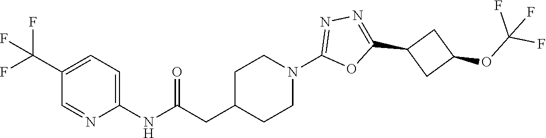 [SMILES: O=C(CC1CCN(c2nnc([C@H]3C[C@@H](OC(F)(F)F)C3)o2)CC1)Nc1ccc(C(F)(F)F)cn1] |
| 125 | 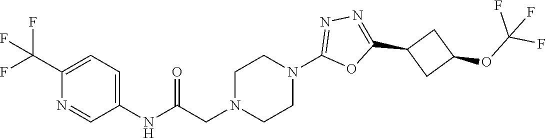 [SMILES: O=C(CN1CCN(c2nnc([C@H]3C[C@@H](OC(F)(F)F)C3)o2)CC1)Nc1ccc(C(F)(F)F)nc1] |
| 126 | 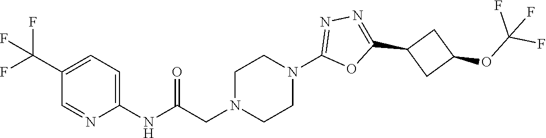 [SMILES: O=C(CN1CCN(c2nnc([C@H]3C[C@@H](OC(F)(F)F)C3)o2)CC1)Nc1ccc(C(F)(F)F)cn1] |
| 127 | 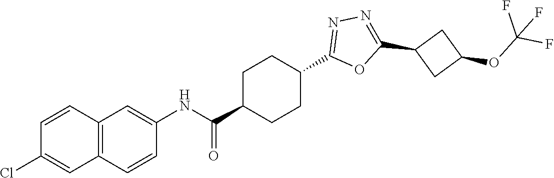 [SMILES: O=C(Nc1ccc2cc(Cl)ccc2c1)[C@H]1CC[C@H](c2nnc([C@H]3C[C@@H](OC(F)(F)F)C3)o2)CC1] |
| 128 | 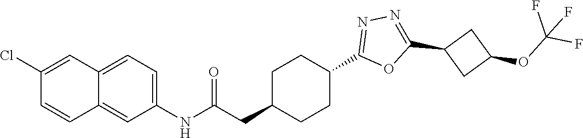 [SMILES: O=C(C[C@H]1CC[C@H](c2nnc([C@H]3C[C@@H](OC(F)(F)F)C3)o2)CC1)Nc1ccc2cc(Cl)ccc2c1] |
| 129 | 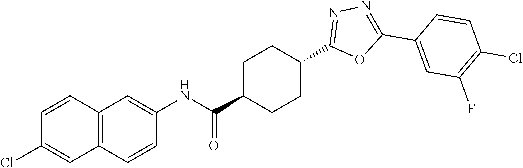 [SMILES: O=C(Nc1ccc2cc(Cl)ccc2c1)[C@H]1CC[C@H](c2nnc(-c3ccc(Cl)c(F)c3)o2)CC1] |
| 130 | 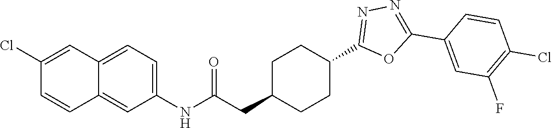 [SMILES: O=C(C[C@H]1CC[C@H](c2nnc(-c3ccc(Cl)c(F)c3)o2)CC1)Nc1ccc2cc(Cl)ccc2c1] |
| 131 | 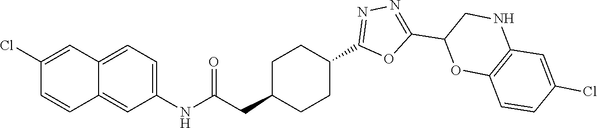 [SMILES: O=C(C[C@H]1CC[C@H](c2nnc(C3CNc4cc(Cl)ccc4O3)o2)CC1)Nc1ccc2cc(Cl)ccc2c1] |
| 132 | 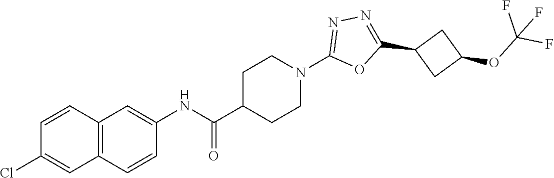 [SMILES: O=C(Nc1ccc2cc(Cl)ccc2c1)C1CCN(c2nnc([C@H]3C[C@@H](OC(F)(F)F)C3)o2)CC1] |
| 133 | 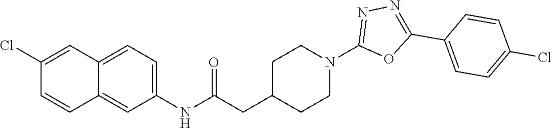 [SMILES: O=C(CC1CCN(c2nnc(-c3ccc(Cl)cc3)o2)CC1)Nc1ccc2cc(Cl)ccc2c1] |
| 134 | 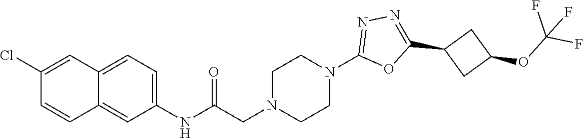 [SMILES: O=C(CN1CCN(c2nnc([C@H]3C[C@@H](OC(F)(F)F)C3)o2)CC1)Nc1ccc2cc(Cl)ccc2c1] |
| 135 | 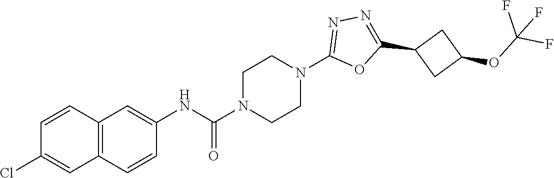 [SMILES: O=C(Nc1ccc2cc(Cl)ccc2c1)N1CCN(c2nnc([C@H]3C[C@@H](OC(F)(F)F)C3)o2)CC1] |
| 136 | 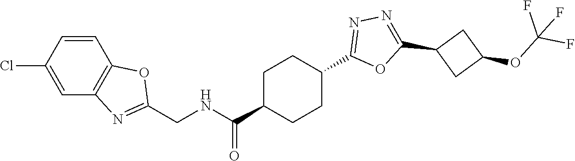 [SMILES: O=C(NCc1nc2cc(Cl)ccc2o1)[C@H]1CC[C@H](c2nnc([C@H]3C[C@@H](OC(F)(F)F)C3)o2)CC1] |
| 137 | 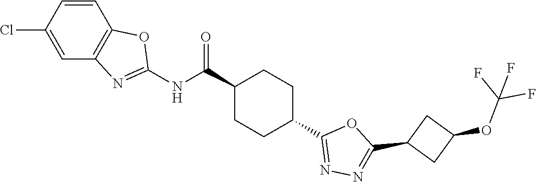 [SMILES: O=C(Nc1nc2cc(Cl)ccc2o1)[C@H]1CC[C@H](c2nnc([C@H]3C[C@@H](OC(F)(F)F)C3)o2)CC1] |
| 138 | 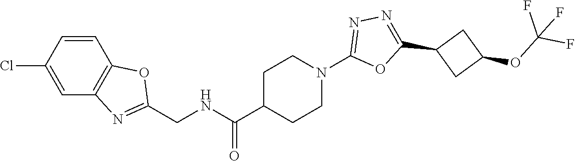 [SMILES: O=C(NCc1nc2cc(Cl)ccc2o1)C1CCN(c2nnc([C@H]3C[C@@H](OC(F)(F)F)C3)o2)CC1] |
| 139 | 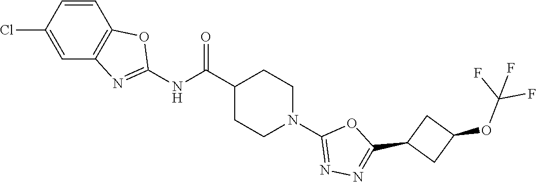 [SMILES: O=C(Nc1nc2cc(Cl)ccc2o1)C1CCN(c2nnc([C@H]3C[C@@H](OC(F)(F)F)C3)o2)CC1] |
| 140 | 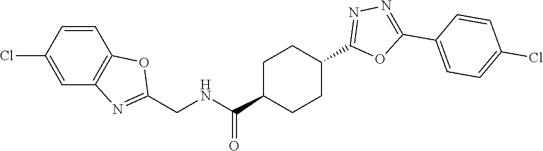 [SMILES: O=C(NCc1nc2cc(Cl)ccc2o1)[C@H]1CC[C@H](c2nnc(-c3ccc(Cl)cc3)o2)CC1] |
| 141 | 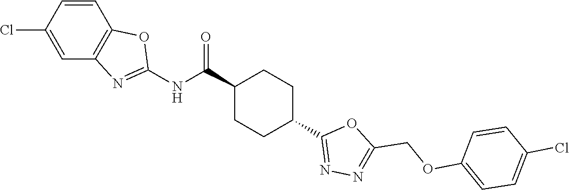 [SMILES: O=C(Nc1nc2cc(Cl)ccc2o1)[C@H]1CC[C@H](c2nnc(COc3ccc(Cl)cc3)o2)CC1] |
| 142 | 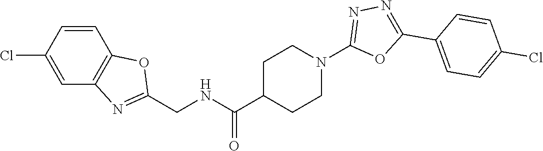 [SMILES: O=C(NCc1nc2cc(Cl)ccc2o1)C1CCN(c2nnc(-c3ccc(Cl)cc3)o2)CC1] |
| 143 | 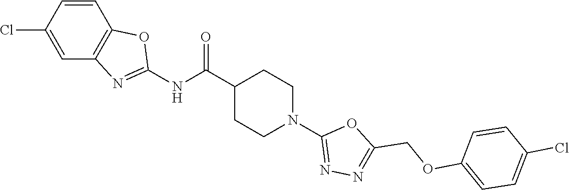 [SMILES: O=C(Nc1nc2cc(Cl)ccc2o1)C1CCN(c2nnc(COc3ccc(Cl)cc3)o2)CC1] |
| 144 | 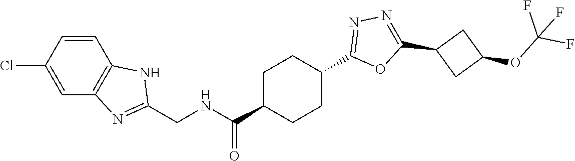 [SMILES: O=C(NCc1nc2cc(Cl)ccc2[nH]1)[C@H]1CC[C@H](c2nnc([C@H]3C[C@@H](OC(F)(F)F)C3)o2)CC1] |
| 145 | 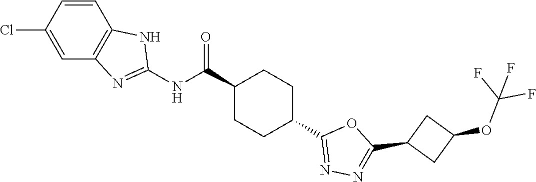 [SMILES: O=C(Nc1nc2cc(Cl)ccc2[nH]1)[C@H]1CC[C@H](c2nnc([C@H]3C[C@@H](OC(F)(F)F)C3)o2)CC1] |
| 146 | 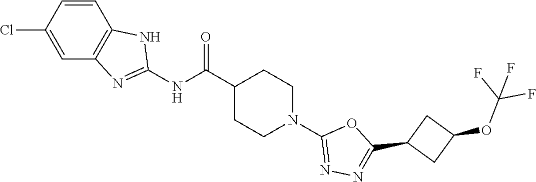 [SMILES: O=C(Nc1nc2cc(Cl)ccc2[nH]1)C1CCN(c2nnc([C@H]3C[C@@H](OC(F)(F)F)C3)o2)CC1] |
| 147 | 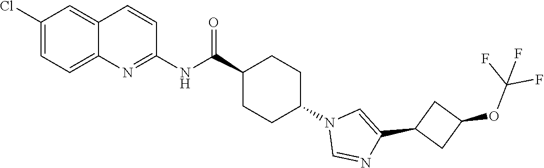 [SMILES: O=C(Nc1ccc2cc(Cl)ccc2n1)[C@H]1CC[C@H](n2cnc([C@H]3C[C@@H](OC(F)(F)F)C3)c2)CC1] |
| 148 | 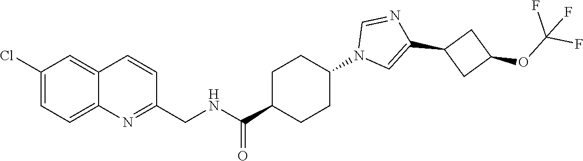 [SMILES: O=C(NCc1ccc2cc(Cl)ccc2n1)[C@H]1CC[C@H](n2cnc([C@H]3C[C@@H](OC(F)(F)F)C3)c2)CC1] |
| 149 | 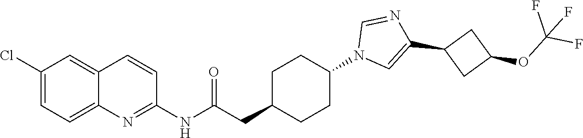 [SMILES: O=C(C[C@H]1CC[C@H](n2cnc([C@H]3C[C@@H](OC(F)(F)F)C3)c2)CC1)Nc1ccc2cc(Cl)ccc2n1] |
| 150 | 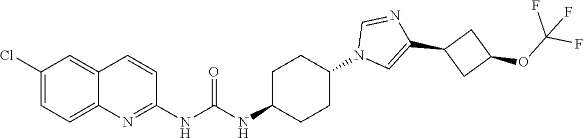 [SMILES: O=C(Nc1ccc2cc(Cl)ccc2n1)N[C@H]1CC[C@H](n2cnc([C@H]3C[C@@H](OC(F)(F)F)C3)c2)CC1] |
| 151 | 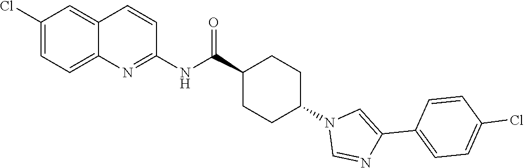 [SMILES: O=C(Nc1ccc2cc(Cl)ccc2n1)[C@H]1CC[C@H](n2cnc(-c3ccc(Cl)cc3)c2)CC1] |
| 152 | 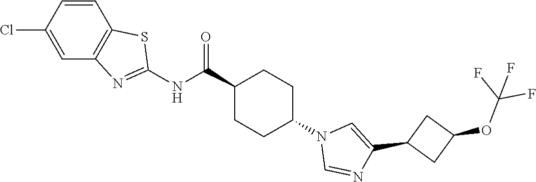 [SMILES: O=C(Nc1nc2cc(Cl)ccc2s1)[C@H]1CC[C@H](n2cnc([C@H]3C[C@@H](OC(F)(F)F)C3)c2)CC1] |
| 153 | 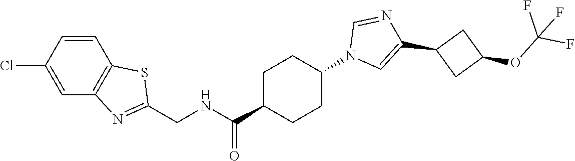 [SMILES: O=C(NCc1nc2cc(Cl)ccc2s1)[C@H]1CC[C@H](n2cnc([C@H]3C[C@@H](OC(F)(F)F)C3)c2)CC1] |
| 154 | 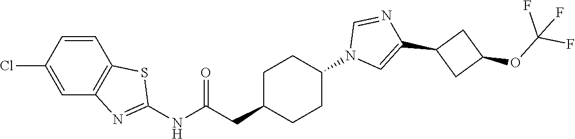 [SMILES: O=C(C[C@H]1CC[C@H](n2cnc([C@H]3C[C@@H](OC(F)(F)F)C3)c2)CC1)Nc1nc2cc(Cl)ccc2s1] |
| 155 | 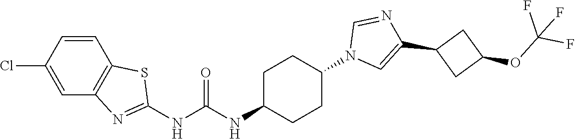 [SMILES: O=C(Nc1nc2cc(Cl)ccc2s1)N[C@H]1CC[C@H](n2cnc([C@H]3C[C@@H](OC(F)(F)F)C3)c2)CC1] |
| 156 | 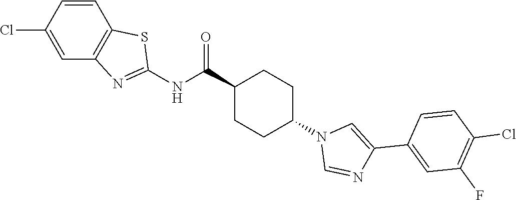 [SMILES: O=C(Nc1nc2cc(Cl)ccc2s1)[C@H]1CC[C@H](n2cnc(-c3ccc(Cl)c(F)c3)c2)CC1] |
| 157 | 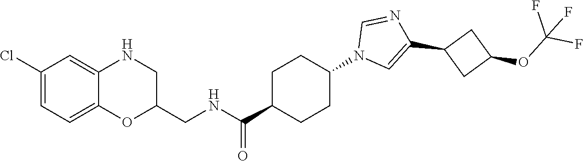 [SMILES: O=C(NCC1CNc2cc(Cl)ccc2O1)[C@H]1CC[C@H](n2cnc([C@H]3C[C@@H](OC(F)(F)F)C3)c2)CC1] |
| 158 | 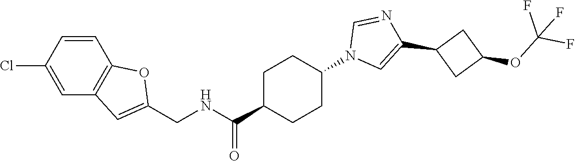 [SMILES: O=C(NCc1cc2cc(Cl)ccc2o1)[C@H]1CC[C@H](n2cnc([C@H]3C[C@@H](OC(F)(F)F)C3)c2)CC1] |
| 159 | 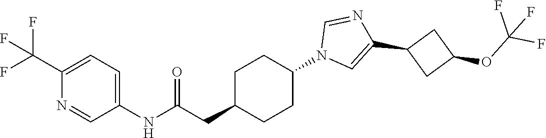 [SMILES: O=C(C[C@H]1CC[C@H](n2cnc([C@H]3C[C@@H](OC(F)(F)F)C3)c2)CC1)Nc1ccc(C(F)(F)F)nc1] |
| 160 | 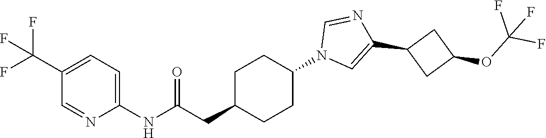 [SMILES: O=C(C[C@H]1CC[C@H](n2cnc([C@H]3C[C@@H](OC(F)(F)F)C3)c2)CC1)Nc1ccc(C(F)(F)F)cn1] |
| 161 | 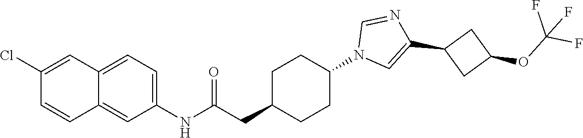 [SMILES: O=C(C[C@H]1CC[C@H](n2cnc([C@H]3C[C@@H](OC(F)(F)F)C3)c2)CC1)Nc1ccc2cc(Cl)ccc2c1] |
| 162 | 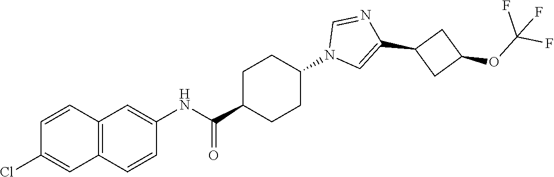 [SMILES: O=C(Nc1ccc2cc(Cl)ccc2c1)[C@H]1CC[C@H](n2cnc([C@H]3C[C@@H](OC(F)(F)F)C3)c2)CC1] |
| 163 | 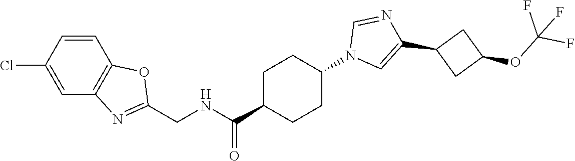 [SMILES: O=C(NCc1nc2cc(Cl)ccc2o1)[C@H]1CC[C@H](n2cnc([C@H]3C[C@@H](OC(F)(F)F)C3)c2)CC1] |
| 164 | 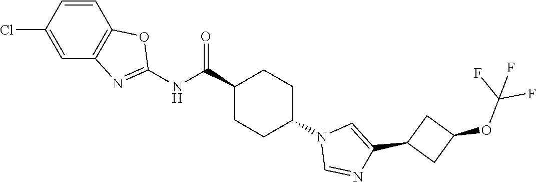 [SMILES: O=C(Nc1nc2cc(Cl)ccc2o1)[C@H]1CC[C@H](n2cnc([C@H]3C[C@@H](OC(F)(F)F)C3)c2)CC1] |
| 165 | 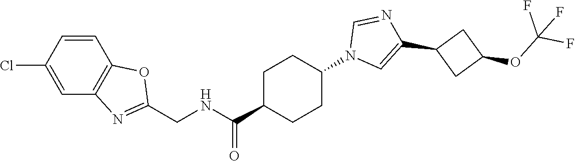 [SMILES: O=C(NCc1nc2cc(Cl)ccc2o1)[C@H]1CC[C@H](n2cnc([C@H]3C[C@@H](OC(F)(F)F)C3)c2)CC1] |
| 166 | 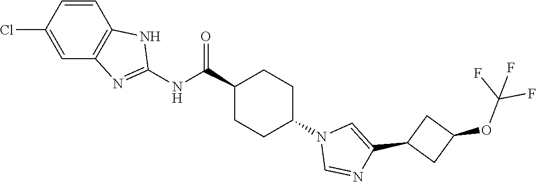 [SMILES: O=C(Nc1nc2cc(Cl)ccc2[nH]1)[C@H]1CC[C@H](n2cnc([C@H]3C[C@@H](OC(F)(F)F)C3)c2)CC1] |
| 167 | 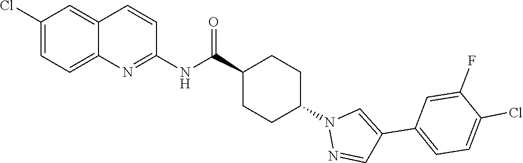 [SMILES: O=C(Nc1ccc2cc(Cl)ccc2n1)[C@H]1CC[C@H](n2cc(-c3ccc(Cl)c(F)c3)cn2)CC1] |
| 168 | 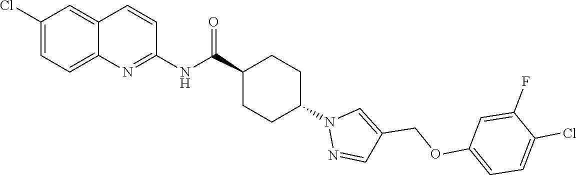 [SMILES: O=C(Nc1ccc2cc(Cl)ccc2n1)[C@H]1CC[C@H](n2cc(COc3ccc(Cl)c(F)c3)cn2)CC1] |
| 169 | 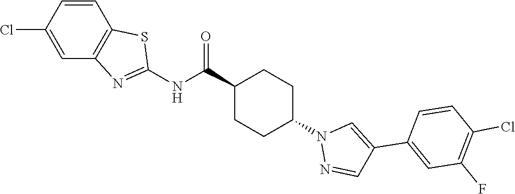 [SMILES: O=C(Nc1nc2cc(Cl)ccc2s1)[C@H]1CC[C@H](n2cc(-c3ccc(Cl)c(F)c3)cn2)CC1] |
| 170 | 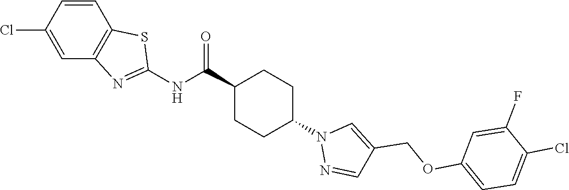 [SMILES: O=C(Nc1nc2cc(Cl)ccc2s1)[C@H]1CC[C@H](n2cc(COc3ccc(Cl)c(F)c3)cn2)CC1] |
| 171 | 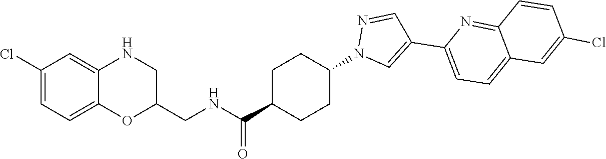 [SMILES: O=C(NCC1CNc2cc(Cl)ccc2O1)[C@H]1CC[C@H](n2cc(-c3ccc4cc(Cl)ccc4n3)cn2)CC1] |
| 172 | 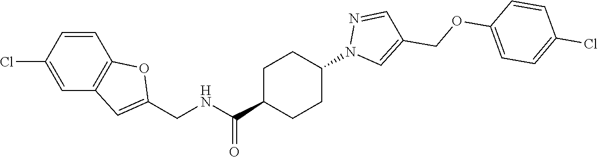 [SMILES: O=C(NCc1cc2cc(Cl)ccc2o1)[C@H]1CC[C@H](n2cc(COc3ccc(Cl)cc3)cn2)CC1] |
| 173 | 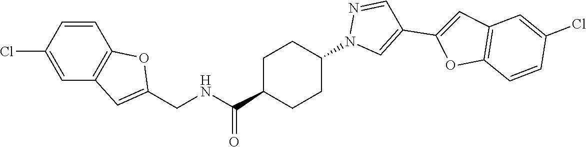 [SMILES: O=C(NCc1cc2cc(Cl)ccc2o1)[C@H]1CC[C@H](n2cc(-c3cc4cc(Cl)ccc4o3)cn2)CC1] |
| 174 | 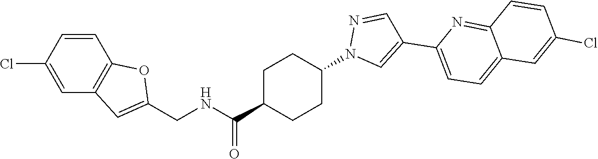 [SMILES: O=C(NCc1cc2cc(Cl)ccc2o1)[C@H]1CC[C@H](n2cc(-c3ccc4cc(Cl)ccc4n3)cn2)CC1] |
| 175 | 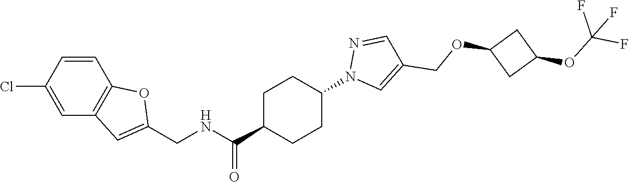 [SMILES: O=C(NCc1cc2cc(Cl)ccc2o1)[C@H]1CC[C@H](n2cc(CO[C@H]3C[C@@H](OC(F)(F)F)C3)cn2)CC1] |
| 176 | 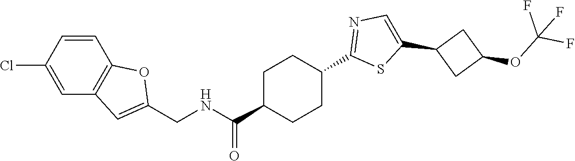 [SMILES: O=C(NCc1cc2cc(Cl)ccc2o1)[C@H]1CC[C@H](c2ncc([C@H]3C[C@@H](OC(F)(F)F)C3)s2)CC1] |
| 177 | 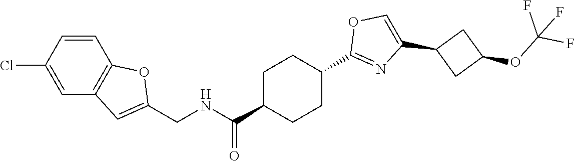 [SMILES: O=C(NCc1cc2cc(Cl)ccc2o1)[C@H]1CC[C@H](c2nc([C@H]3C[C@@H](OC(F)(F)F)C3)co2)CC1] |
| 178 | 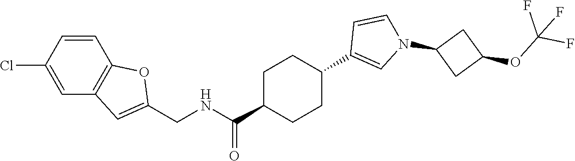 [SMILES: O=C(NCc1cc2cc(Cl)ccc2o1)[C@H]1CC[C@H](c2ccn([C@H]3C[C@@H](OC(F)(F)F)C3)c2)CC1] |
| 179 | 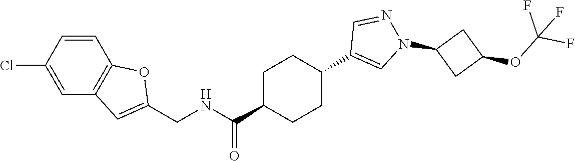 [SMILES: O=C(NCc1cc2cc(Cl)ccc2o1)[C@H]1CC[C@H](c2cnn([C@H]3C[C@@H](OC(F)(F)F)C3)c2)CC1] |
| 180 | 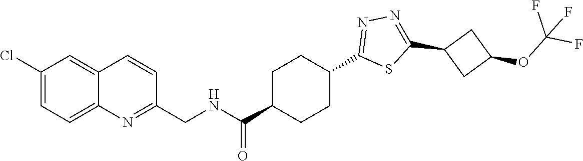 [SMILES: O=C(NCc1ccc2cc(Cl)ccc2n1)[C@H]1CC[C@H](c2nnc([C@H]3C[C@@H](OC(F)(F)F)C3)s2)CC1] |
| 181 | 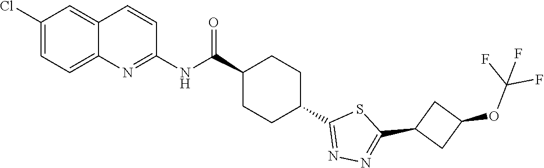 [SMILES: O=C(Nc1ccc2cc(Cl)ccc2n1)[C@H]1CC[C@H](c2nnc([C@H]3C[C@@H](OC(F)(F)F)C3)s2)CC1] |
| 182 | 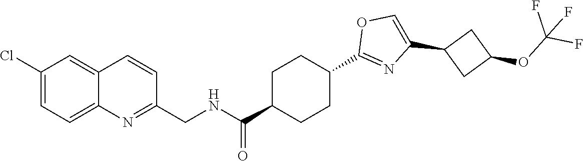 [SMILES: O=C(NCc1ccc2cc(Cl)ccc2n1)[C@H]1CC[C@H](c2nc([C@H]3C[C@@H](OC(F)(F)F)C3)co2)CC1] |
| 183 | 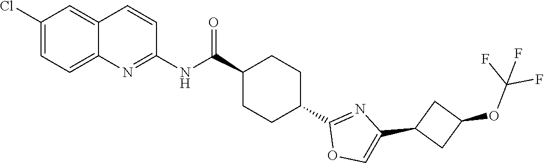 [SMILES: O=C(Nc1ccc2cc(Cl)ccc2n1)[C@H]1CC[C@H](c2nc([C@H]3C[C@@H](OC(F)(F)F)C3)co2)CC1] |
| 184 | 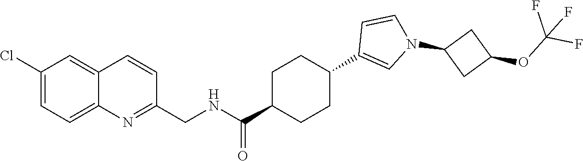 [SMILES: O=C(NCc1ccc2cc(Cl)ccc2n1)[C@H]1CC[C@H](c2ccn([C@H]3C[C@@H](OC(F)(F)F)C3)c2)CC1] |
| 185 | 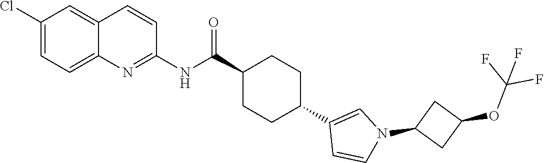 [SMILES: O=C(Nc1ccc2cc(Cl)ccc2n1)[C@H]1CC[C@H](c2ccn([C@H]3C[C@@H](OC(F)(F)F)C3)c2)CC1] |
| 186 | 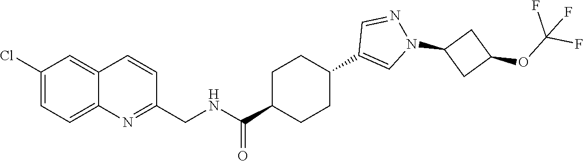 [SMILES: O=C(NCc1ccc2cc(Cl)ccc2n1)[C@H]1CC[C@H](c2cnn([C@H]3C[C@@H](OC(F)(F)F)C3)c2)CC1] |
| 187 | 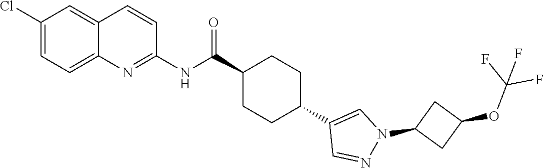 [SMILES: O=C(Nc1ccc2cc(Cl)ccc2n1)[C@H]1CC[C@H](c2cnn([C@H]3C[C@@H](OC(F)(F)F)C3)c2)CC1] |
| 188 | 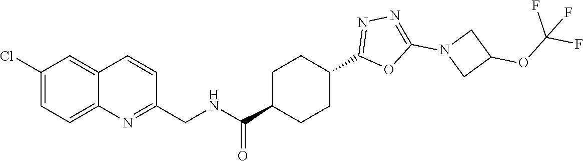 [SMILES: O=C(NCc1ccc2cc(Cl)ccc2n1)[C@H]1CC[C@H](c2nnc(N3CC(OC(F)(F)F)C3)o2)CC1] |
| 189 | 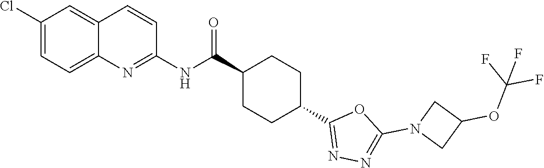 [SMILES: O=C(Nc1ccc2cc(Cl)ccc2n1)[C@H]1CC[C@H](c2nnc(N3CC(OC(F)(F)F)C3)o2)CC1] |
| 190 | 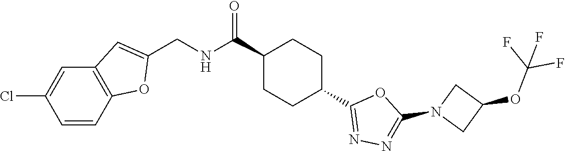 |
| 191 | 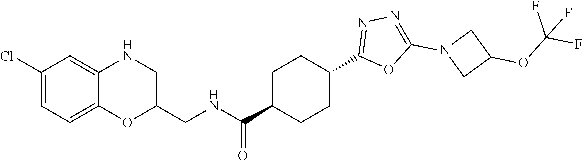 [SMILES: O=C(NCC1CNc2cc(Cl)ccc2O1)[C@H]1CC[C@H](c2nnc(N3CC(OC(F)(F)F)C3)o2)CC1] |
| 192 | 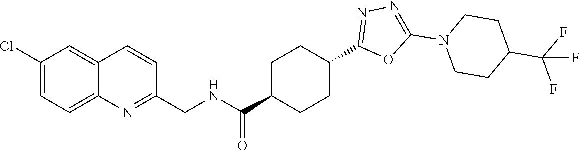 [SMILES: O=C(NCc1ccc2cc(Cl)ccc2n1)[C@H]1CC[C@H](c2nnc(N3CCC(C(F)(F)F)CC3)o2)CC1] |
| 193 | 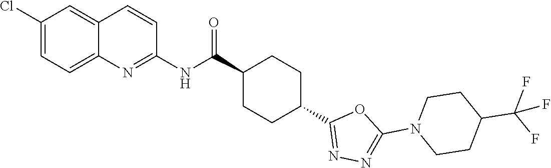 [SMILES: O=C(Nc1ccc2cc(Cl)ccc2n1)[C@H]1CC[C@H](c2nnc(N3CCC(C(F)(F)F)CC3)o2)CC1] |
| 194 | 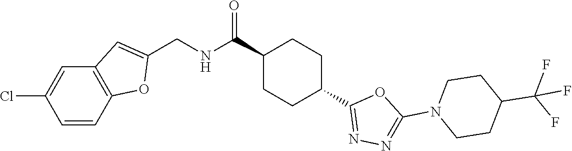 [SMILES: O=C(NCc1cc2cc(Cl)ccc2o1)[C@H]1CC[C@H](c2nnc(N3CCC(C(F)(F)F)CC3)o2)CC1] |
| 195 | 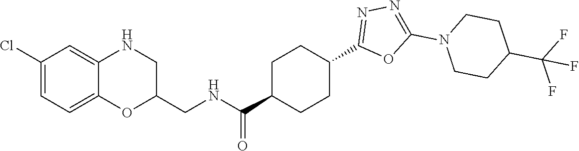 [SMILES: O=C(NCC1CNc2cc(Cl)ccc2O1)[C@H]1CC[C@H](c2nnc(N3CCC(C(F)(F)F)CC3)o2)CC1] |
| 196 | 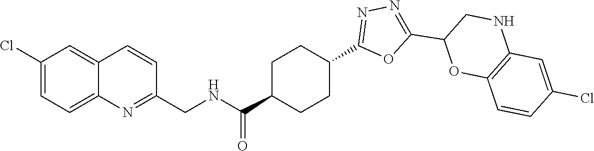 [SMILES: O=C(NCc1ccc2cc(Cl)ccc2n1)[C@H]1CC[C@H](c2nnc(C3CNc4cc(Cl)ccc4O3)o2)CC1] |
| 197 | 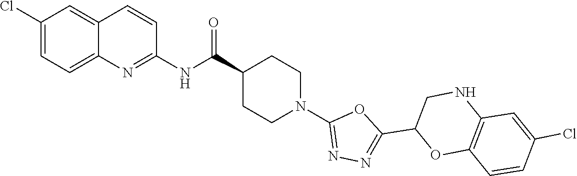 |
| 198 | 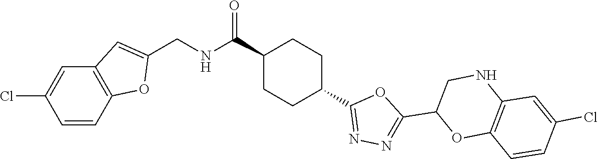 [SMILES: O=C(NCc1cc2cc(Cl)ccc2o1)[C@H]1CC[C@H](c2nnc(C3CNc4cc(Cl)ccc4O3)o2)CC1] |
| 199 | 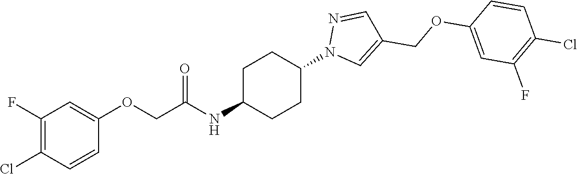 [SMILES: O=C(COc1ccc(Cl)c(F)c1)N[C@H]1CC[C@H](n2cc(COc3ccc(Cl)c(F)c3)cn2)CC1] |
| 200 | 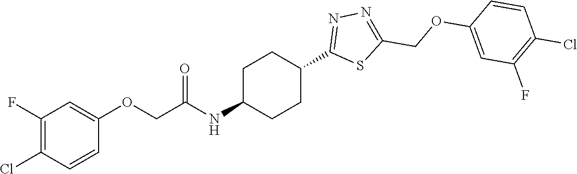 [SMILES: O=C(COc1ccc(Cl)c(F)c1)N[C@H]1CC[C@H](c2nnc(COc3ccc(Cl)c(F)c3)s2)CC1] |
| 201 | 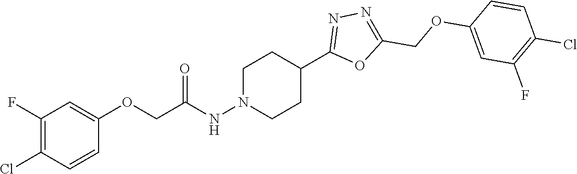 [SMILES: O=C(COc1ccc(Cl)c(F)c1)NN1CCC(c2nnc(COc3ccc(Cl)c(F)c3)o2)CC1] |
| 202 | 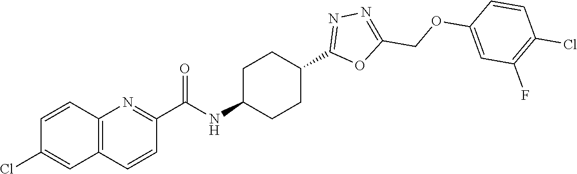 [SMILES: O=C(N[C@H]1CC[C@H](c2nnc(COc3ccc(Cl)c(F)c3)o2)CC1)c1ccc2cc(Cl)ccc2n1] |
| 203 | 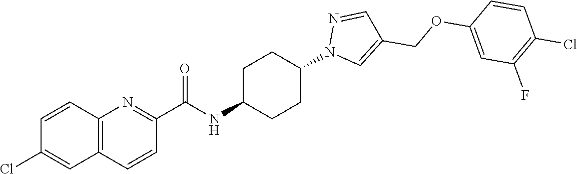 [SMILES: O=C(N[C@H]1CC[C@H](n2cc(COc3ccc(Cl)c(F)c3)cn2)CC1)c1ccc2cc(Cl)ccc2n1] |
| 204 | 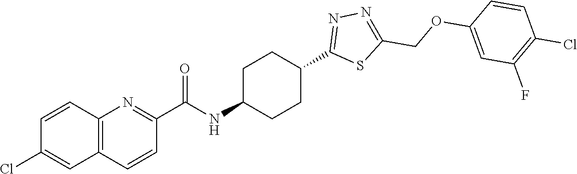 [SMILES: O=C(N[C@H]1CC[C@H](c2nnc(COc3ccc(Cl)c(F)c3)s2)CC1)c1ccc2cc(Cl)ccc2n1] |
| 205 | 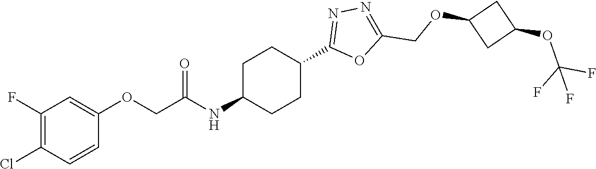 [SMILES: O=C(COc1ccc(Cl)c(F)c1)N[C@H]1CC[C@H](c2nnc(CO[C@H]3C[C@@H](OC(F)(F)F)C3)o2)CC1] |
| 206 | 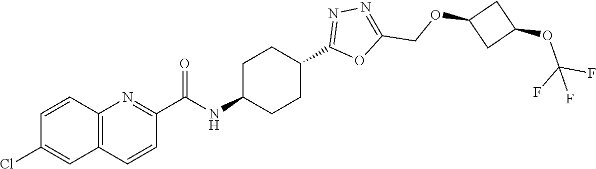 [SMILES: O=C(N[C@H]1CC[C@H](c2nnc(CO[C@H]3C[C@@H](OC(F)(F)F)C3)o2)CC1)c1ccc2cc(Cl)ccc2n1] |
| 207 | 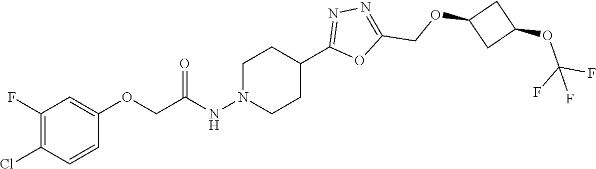 [SMILES: O=C(COc1ccc(Cl)c(F)c1)NN1CCC(c2nnc(CO[C@H]3C[C@@H](OC(F)(F)F)C3)o2)CC1] |
| 208 | 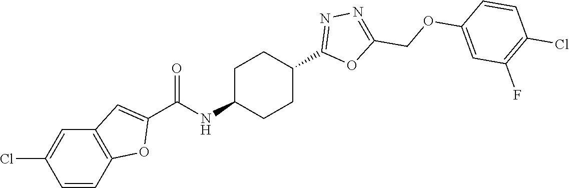 [SMILES: O=C(N[C@H]1CC[C@H](c2nnc(COc3ccc(Cl)c(F)c3)o2)CC1)c1cc2cc(Cl)ccc2o1] |
| 209 | 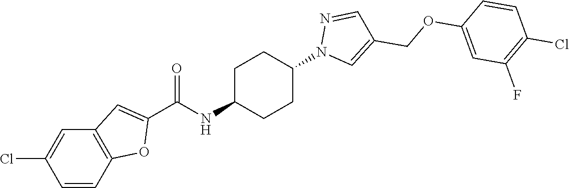 [SMILES: O=C(N[C@H]1CC[C@H](n2cc(COc3ccc(Cl)c(F)c3)cn2)CC1)c1cc2cc(Cl)ccc2o1] |
| 210 | 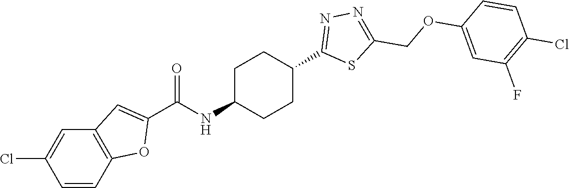 [SMILES: O=C(N[C@H]1CC[C@H](c2nnc(COc3ccc(Cl)c(F)c3)s2)CC1)c1cc2cc(Cl)ccc2o1] |
| 211 | 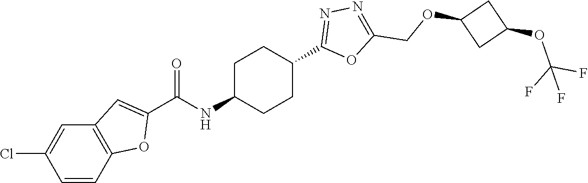 [SMILES: O=C(N[C@H]1CC[C@H](c2nnc(CO[C@H]3C[C@@H](OC(F)(F)F)C3)o2)CC1)c1cc2cc(Cl)ccc2o1] |
| 212 | 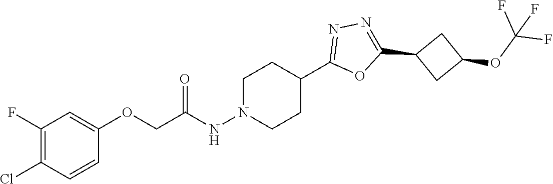 [SMILES: O=C(COc1ccc(Cl)c(F)c1)NN1CCC(c2nnc([C@H]3C[C@@H](OC(F)(F)F)C3)o2)CC1] |
| 213 | 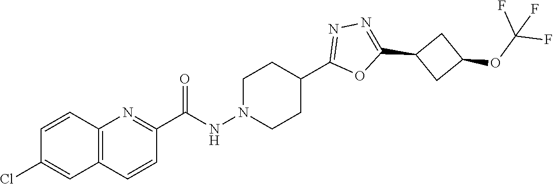 [SMILES: O=C(NN1CCC(c2nnc([C@H]3C[C@@H](OC(F)(F)F)C3)o2)CC1)c1ccc2cc(Cl)ccc2n1] |
| 214 | 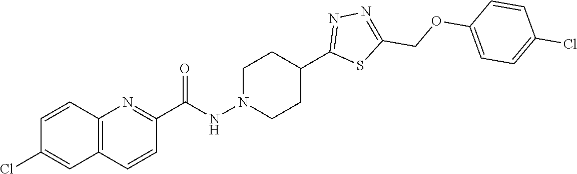 [SMILES: O=C(NN1CCC(c2nnc(COc3ccc(Cl)cc3)s2)CC1)c1ccc2cc(Cl)ccc2n1] |
| 215 | 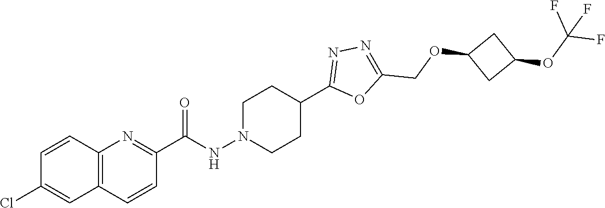 [SMILES: O=C(NN1CCC(c2nnc(CO[C@H]3C[C@@H](OC(F)(F)F)C3)o2)CC1)c1ccc2cc(Cl)ccc2n1] |
| 216 | 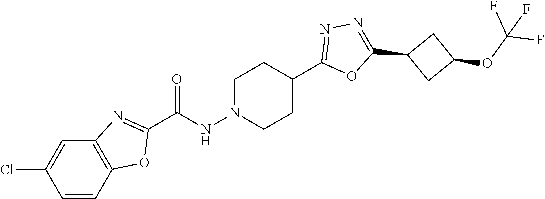 [SMILES: O=C(NN1CCC(c2nnc([C@H]3C[C@@H](OC(F)(F)F)C3)o2)CC1)c1nc2cc(Cl)ccc2o1] |
| 217 | 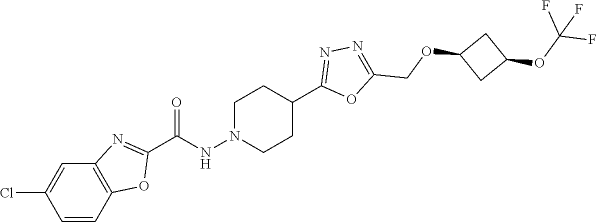 [SMILES: O=C(NN1CCC(c2nnc(CO[C@H]3C[C@@H](OC(F)(F)F)C3)o2)CC1)c1nc2cc(Cl)ccc2o1] |
| 218 | 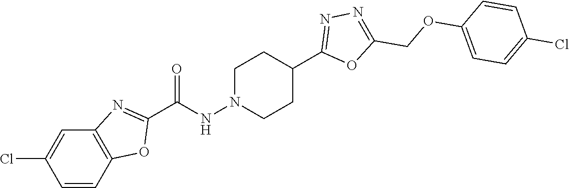 [SMILES: O=C(NN1CCC(c2nnc(COc3ccc(Cl)cc3)o2)CC1)c1nc2cc(Cl)ccc2o1] |
| 219 | 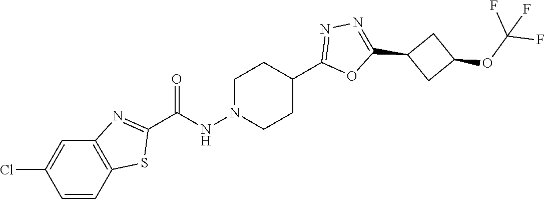 [SMILES: O=C(NN1CCC(c2nnc([C@H]3C[C@@H](OC(F)(F)F)C3)o2)CC1)c1nc2cc(Cl)ccc2s1] |
| 220 | 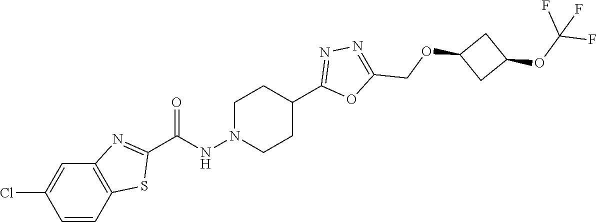 [SMILES: O=C(NN1CCC(c2nnc(CO[C@H]3C[C@@H](OC(F)(F)F)C3)o2)CC1)c1nc2cc(Cl)ccc2s1] |
| 221 | 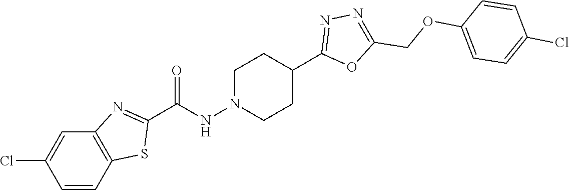 [SMILES: O=C(NN1CCC(c2nnc(COc3ccc(Cl)cc3)o2)CC1)c1nc2cc(Cl)ccc2s1] |
| 222 | 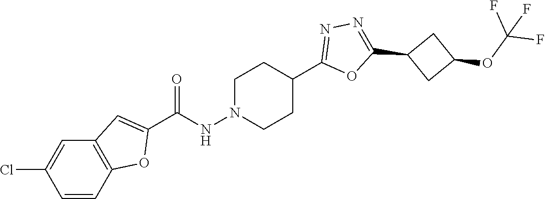 [SMILES: O=C(NN1CCC(c2nnc([C@H]3C[C@@H](OC(F)(F)F)C3)o2)CC1)c1cc2cc(Cl)ccc2o1] |
| 223 | 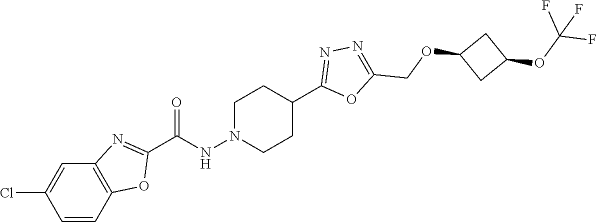 [SMILES: O=C(NN1CCC(c2nnc(CO[C@H]3C[C@@H](OC(F)(F)F)C3)o2)CC1)c1nc2cc(Cl)ccc2o1] |
| 224 | 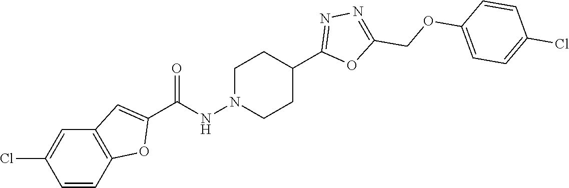 [SMILES: O=C(NN1CCC(c2nnc(COc3ccc(Cl)cc3)o2)CC1)c1cc2cc(Cl)ccc2o1] |
| 225 | 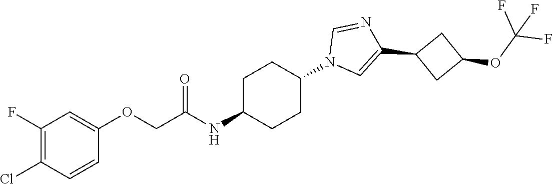 [SMILES: O=C(COc1ccc(Cl)c(F)c1)N[C@H]1CC[C@H](n2cnc([C@H]3C[C@@H](OC(F)(F)F)C3)c2)CC1] |
| 226 | 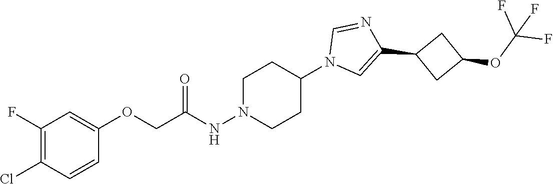 [SMILES: O=C(COc1ccc(Cl)c(F)c1)NN1CCC(n2cnc([C@H]3C[C@@H](OC(F)(F)F)C3)c2)CC1] |
| 227 | 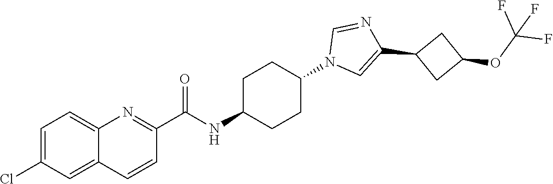 [SMILES: O=C(N[C@H]1CC[C@H](n2cnc([C@H]3C[C@@H](OC(F)(F)F)C3)c2)CC1)c1ccc2cc(Cl)ccc2n1] |
| 228 | 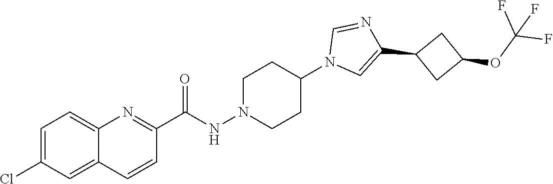 [SMILES: O=C(NN1CCC(n2cnc([C@H]3C[C@@H](OC(F)(F)F)C3)c2)CC1)c1ccc2cc(Cl)ccc2n1] |
| 229 | 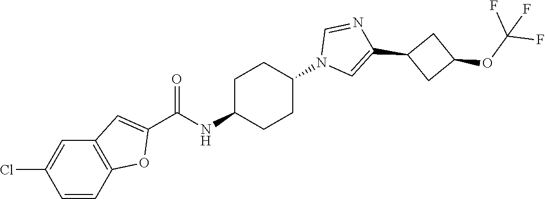 [SMILES: O=C(N[C@H]1CC[C@H](n2cnc([C@H]3C[C@@H](OC(F)(F)F)C3)c2)CC1)c1cc2cc(Cl)ccc2o1] |
| 230 | 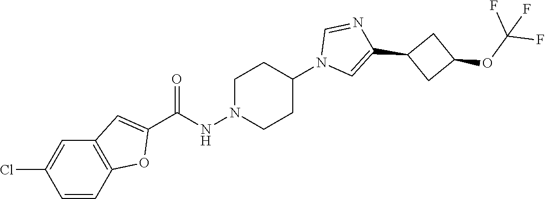 [SMILES: O=C(NN1CCC(n2cnc([C@H]3C[C@@H](OC(F)(F)F)C3)c2)CC1)c1cc2cc(Cl)ccc2o1] |
| 231 | 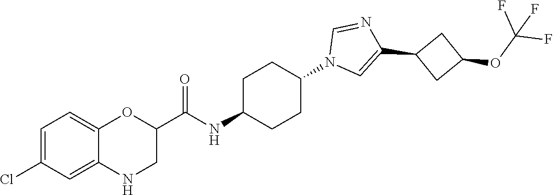 [SMILES: O=C(N[C@H]1CC[C@H](n2cnc([C@H]3C[C@@H](OC(F)(F)F)C3)c2)CC1)C1CNc2cc(Cl)ccc2O1] |
| 232 | 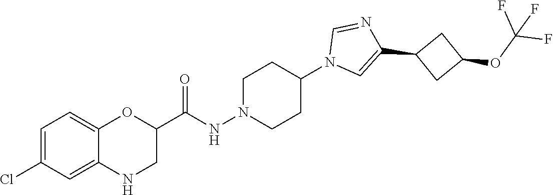 [SMILES: O=C(NN1CCC(n2cnc([C@H]3C[C@@H](OC(F)(F)F)C3)c2)CC1)C1CNc2cc(Cl)ccc2O1] |
| 233 | 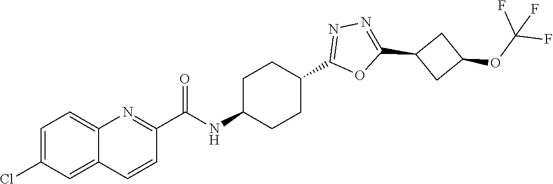 [SMILES: O=C(N[C@H]1CC[C@H](c2nnc([C@H]3C[C@@H](OC(F)(F)F)C3)o2)CC1)c1ccc2cc(Cl)ccc2n1] |
| 234 | 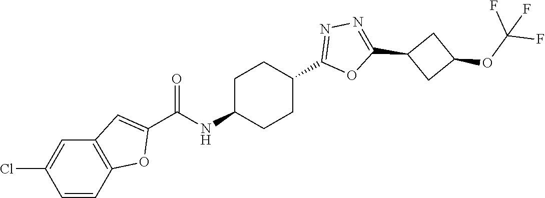 [SMILES: O=C(N[C@H]1CC[C@H](c2nnc([C@H]3C[C@@H](OC(F)(F)F)C3)o2)CC1)c1cc2cc(Cl)ccc2o1] |
| 235 | 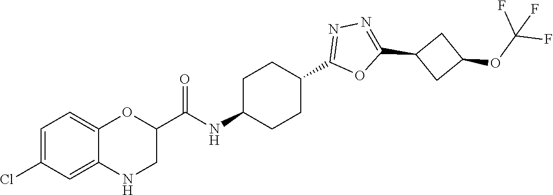 [SMILES: O=C(N[C@H]1CC[C@H](c2nnc([C@H]3C[C@@H](OC(F)(F)F)C3)o2)CC1)C1CNc2cc(Cl)ccc2O1] |
| 236 | 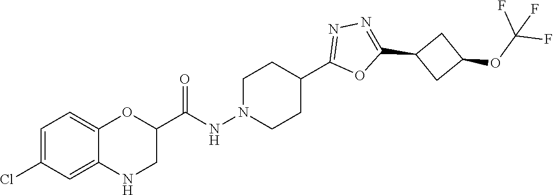 [SMILES: O=C(NN1CCC(c2nnc([C@H]3C[C@@H](OC(F)(F)F)C3)o2)CC1)C1CNc2cc(Cl)ccc2O1] |

TABLE 4

| Compound | |
|---|---|
| No. | Structure |
| 237 | 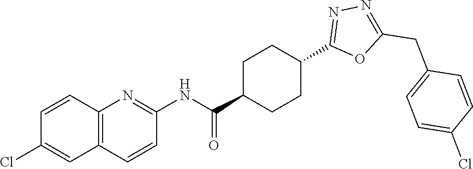 [SMILES: O=C(Nc1ccc2cc(Cl)ccc2n1)[C@H]1CC[C@H](c2nnc(Cc3ccc(Cl)cc3)o2)CC1] |
| 238 | 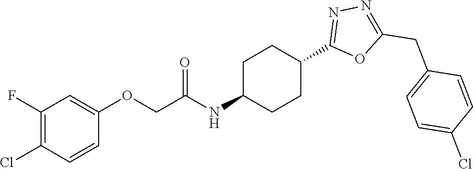 [SMILES: O=C(COc1ccc(Cl)c(F)c1)N[C@H]1CC[C@H](c2nnc(Cc3ccc(Cl)cc3)o2)CC1] |
| 239 | 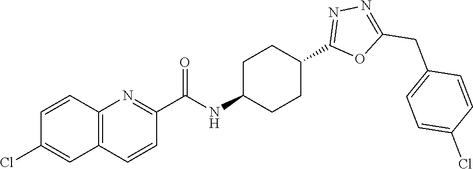 [SMILES: O=C(N[C@H]1CC[C@H](c2nnc(Cc3ccc(Cl)cc3)o2)CC1)c1ccc2cc(Cl)ccc2n1] |
| 240 | 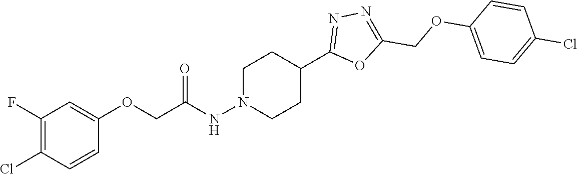 [SMILES: O=C(COc1ccc(Cl)c(F)c1)NN1CCC(c2nnc(COc3ccc(Cl)cc3)o2)CC1] |
| 241 | 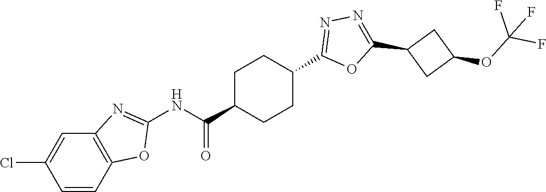 [SMILES: O=C(Nc1nc2cc(Cl)ccc2o1)[C@H]1CC[C@H](c2nnc([C@H]3C[C@@H](OC(F)(F)F)C3)o2)CC1] |
| 242 | 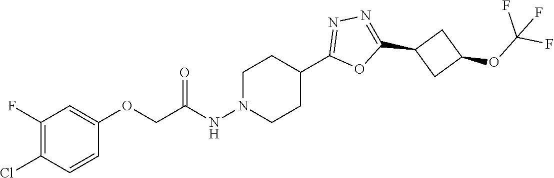 [SMILES: O=C(COc1ccc(Cl)c(F)c1)NN1CCC(c2nnc([C@H]3C[C@@H](OC(F)(F)F)C3)o2)CC1] |
| 243 | 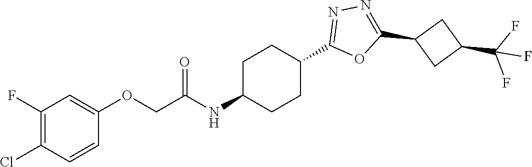 [SMILES: O=C(COc1ccc(Cl)c(F)c1)N[C@H]1CC[C@H](c2nnc([C@H]3C[C@@H](C(F)(F)F)C3)o2)CC1] |
| 244 | 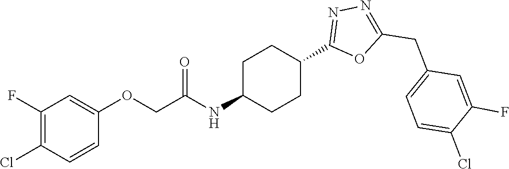 [SMILES: O=C(COc1ccc(Cl)c(F)c1)N[C@H]1CC[C@H](c2nnc(Cc3ccc(Cl)c(F)c3)o2)CC1] |
| 245 | 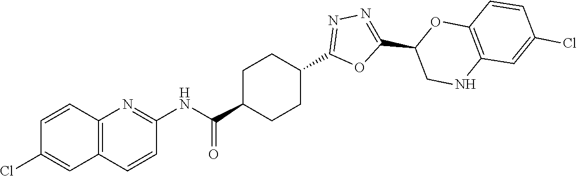 [SMILES: O=C(Nc1ccc2cc(Cl)ccc2n1)[C@H]1CC[C@H](c2nnc([C@@H]3CNc4cc(Cl)ccc4O3)o2)CC1] |
| 246 | 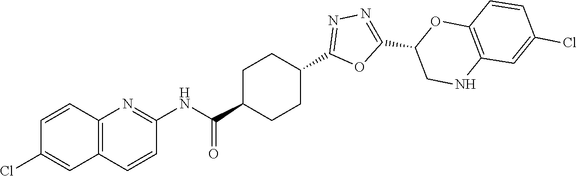 [SMILES: O=C(Nc1ccc2cc(Cl)ccc2n1)[C@H]1CC[C@H](c2nnc([C@H]3CNc4cc(Cl)ccc4O3)o2)CC1] |
| 247 | 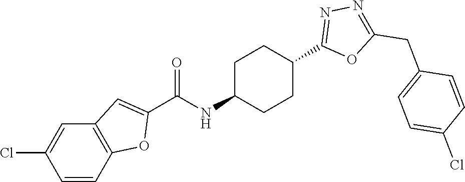 [SMILES: O=C(N[C@H]1CC[C@H](c2nnc(Cc3ccc(Cl)cc3)o2)CC1)c1cc2cc(Cl)ccc2o1] |
| 248 | 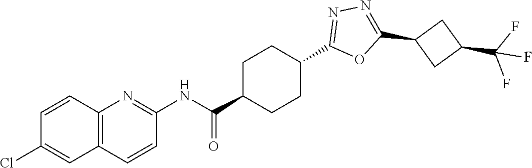 [SMILES: O=C(Nc1ccc2cc(Cl)ccc2n1)[C@H]1CC[C@H](c2nnc([C@H]3C[C@@H](C(F)(F)F)C3)o2)CC1] |
| 249 | 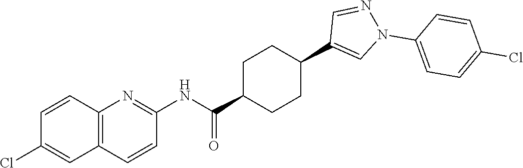 [SMILES: O=C(Nc1ccc2cc(Cl)ccc2n1)[C@H]1CC[C@@H](c2cnn(-c3ccc(Cl)cc3)c2)CC1] |
| 250 | 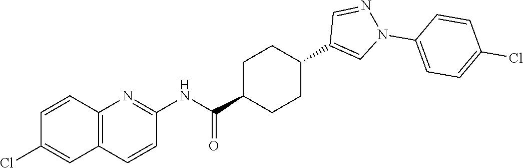 [SMILES: O=C(Nc1ccc2cc(Cl)ccc2n1)[C@H]1CC[C@H](c2cnn(-c3ccc(Cl)cc3)c2)CC1] |
| 251 | 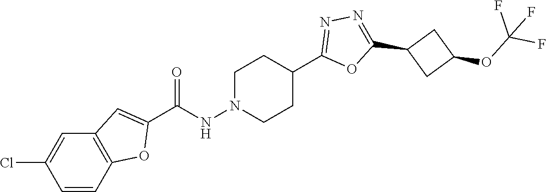 [SMILES: O=C(NN1CCC(c2nnc([C@H]3C[C@@H](OC(F)(F)F)C3)o2)CC1)c1cc2cc(Cl)ccc2o1] |
| 252 | 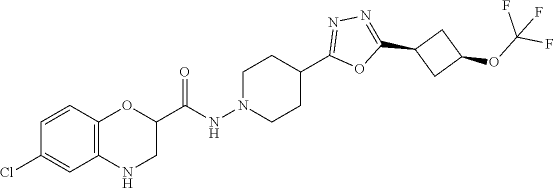 [SMILES: O=C(NN1CCC(c2nnc([C@H]3C[C@@H](OC(F)(F)F)C3)o2)CC1)C1CNc2cc(Cl)ccc2O1] |
| 253 | 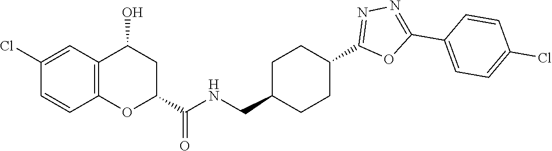 [SMILES: O=C(NC[C@H]1CC[C@H](c2nnc(-c3ccc(Cl)cc3)o2)CC1)[C@H]1C[C@@H](O)c2cc(Cl)ccc2O1] |

TABLE 5

| Compound | |
|---|---|
| No. | Structure |
| 254 | 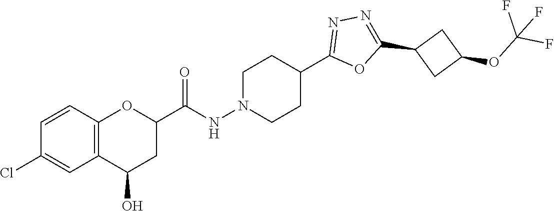 [SMILES: O=C(NN1CCC(c2nnc([C@H]3C[C@@H](OC(F)(F)F)C3)o2)CC1)C1C[C@@H](O)c2cc(Cl)ccc2O1] |
| 255 | 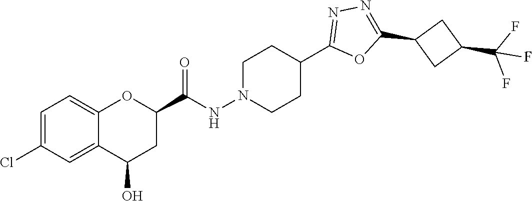 [SMILES: O=C(NN1CCC(c2nnc([C@H]3C[C@@H](C(F)(F)F)C3)o2)CC1)[C@H]1C[C@@H](O)c2cc(Cl)ccc2O1] |
| 256 | 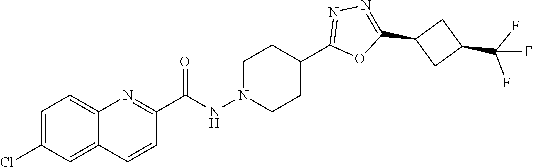 [SMILES: O=C(NN1CCC(c2nnc([C@H]3C[C@@H](C(F)(F)F)C3)o2)CC1)c1ccc2cc(Cl)ccc2n1] |
| 257 | 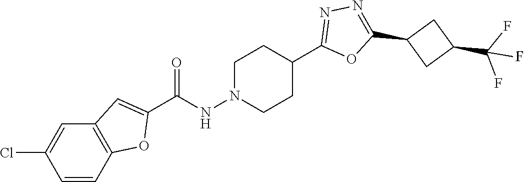 [SMILES: O=C(NN1CCC(c2nnc([C@H]3C[C@@H](C(F)(F)F)C3)o2)CC1)c1cc2cc(Cl)ccc2o1] |
| 258 | 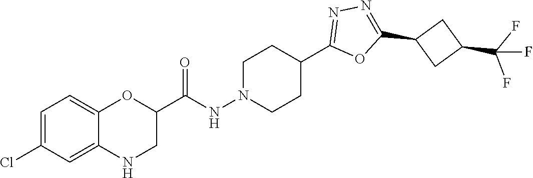 [SMILES: O=C(NN1CCC(c2nnc([C@H]3C[C@@H](C(F)(F)F)C3)o2)CC1)C1CNc2cc(Cl)ccc2O1] |
| 259 | 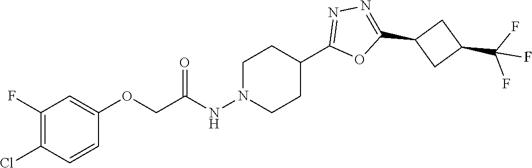 [SMILES: O=C(COc1ccc(Cl)c(F)c1)NN1CCC(c2nnc([C@H]3C[C@@H](C(F)(F)F)C3)o2)CC1] |
| 260 | 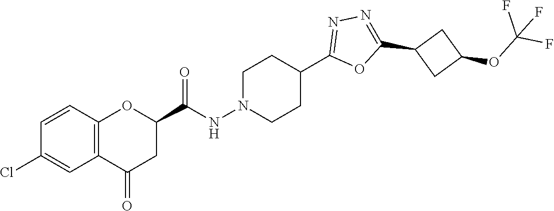 [SMILES: O=C1C[C@H](C(=O)NN2CCC(c3nnc([C@H]4C[C@@H](OC(F)(F)F)C4)o3)CC2)Oc2ccc(Cl)cc21] |
| 261 | 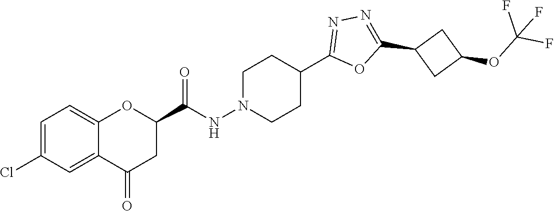 [SMILES: O=C1C[C@H](C(=O)NN2CCC(c3nnc([C@H]4C[C@@H](OC(F)(F)F)C4)o3)CC2)Oc2ccc(Cl)cc21] |

### Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

Agricultural compositions of any of the compounds detailed herein are embraced by this disclosure. Thus, the present disclosure includes agricultural compositions comprising a compound as detailed herein or a agriculturally acceptable salt thereof and a agriculturally acceptable carrier or excipient. In one aspect, the agriculturally acceptable salt is an acid addition salt, such as a salt formed with an inorganic or organic acid. Agricultural compositions may take a form suitable for applying to a plant, such as a for suitable for spraying, chemigation (applying the composition through an irrigation system), granular application, or applying to fertilizer.

Agricultural compositions disclosed herein may comprise excipients or adjuvants, such as solvents, anti-caking agents, stabilizers, defoamers, slip agents, humectants, dispersants, wetting agents, thickening agents, emulsifiers, and preservatives. The agricultural composition may be a concentrated formulation or a ready-to-use formulation.

### Methods of Use and Uses

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound describes herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

The compounds or salts thereof described herein and compositions described herein are believed to be effective for treating a variety of diseases and disorders. In some embodiments, a compound or salt thereof described herein or a composition described herein may be used in a method of treating a disease or disorder mediated by an integrated stress response (ISR) pathway. In some embodiments, the disease or disorder is mediated by eukaryotic translation initiation factor 2a (eIF2a) or eukaryotic translation initiation factor 2B (eIF2B). In some embodiments, the disease or disorder is mediated by phosphorylation of eIF2α and/or the guanine nucleotide exchange factor (GEF) activity of eIF2B. In some embodiments, the disease or disorder is mediated by a decrease in protein synthesis. In some embodiments, the disease or disorder is mediated by the expression of ATF4, ATF3, CHOP, or BACE-1.

In some embodiments, a compound or salt thereof described herein or a composition described herein may be used in a method of treating a disease or disorder, wherein the disease or disorder is a neurodegenerative disease, an inflammatory disease, an autoimmune disease, a metabolic syndrome, a cancer, a vascular disease, a musculoskeletal disease (such as a myopathy), an ocular disease, or a genetic disorder.

In some embodiments, the disease or disorder is a neurodegenerative disease. In some embodiments, the neurodegenerative disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, Pelizaeus-Merzbacher disease, a cognitive impairment, a traumatic brain injury, a postoperative cognitive dysfunction (PCD), a neuro-otological syndrome, hearing loss, Huntington's disease, stroke, chronic traumatic encephalopathy, spinal cord injury, dementia, frontotemporal dementia (FTD), depression, or a social behavior impairment. In some embodiments, the cognitive impairment is triggered by ageing, radiation, sepsis, seizure, heart attack, heart surgery, liver failure, hepatic encephalopathy, anesthesia, brain injury, brain surgery, ischemia, chemotherapy, cancer treatment, critical illness, concussion, fibromyalgia, or depression. In some embodiments, the neurodegenerative disease is Alzheimer's disease. In some embodiments, the neurodegenerative disease is ageing-related cognitive impairment. In some embodiments, the neurodegenerative disease is a traumatic brain injury.

In some embodiments, a compound or salt thereof described herein or a composition described herein may be used in a method of treating Alzheimer's disease. In some embodiments, neurodegeneration, cognitive impairment, and/or amyloidogenesis is decreased.

In some embodiments, the disease or disorder is an inflammatory disease. In some embodiments, the inflammatory disease is arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis, or inflammatory bowel disease. In some embodiments, the inflammatory bowel disease is Crohn' disease, ulcerative colitis, or celiac disease.

In some embodiments, the disease or disorder is an autoimmune disease. In some embodiments, the autoimmune disease is systemic lupus erythematosus, type 1 diabetes, multiple sclerosis, or rheumatoid arthritis.

In some embodiments, the disease or disorder is a metabolic syndrome. In some embodiments, the metabolic syndrome is acute pancreatitis, chronic pancreatitis, alcoholic liver steatosis, obesity, glucose intolerance, insulin resistance, hyperglycemia, fatty liver, dyslipidemia, hyperlipidemia, hyperhomocysteinemia, or type 2 diabetes. In some embodiments, the metabolic syndrome is alcoholic liver steatosis, obesity, glucose intolerance, insulin resistance, hyperglycemia, fatty liver, dyslipidemia, hyperlipidemia, hyperhomocysteinemia, or type 2 diabetes.

In some embodiments, the disease or disorder is a cancer. In some embodiments, the cancer is pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, neuroblastoma, or lung cancer. In some embodiments, the cancer of secretory cells is non-Hodgkin's lymphoma, Burkitt's lymphoma, chronic lymphocytic leukemia, monoclonal gammopathy of undetermined significance (MGUS), plasmocytoma, lymphoplasmacytic lymphoma or acute lymphoblastic leukemia.

In some embodiments, the disease or disorder is a musculoskeletal disease (such as a myopathy). In some embodiments, the musculoskeletal disease is a myopathy, a muscular dystrophy, a muscular atrophy, a muscular wasting, or sarcopenia. In some embodiments, the muscular dystrophy is Duchenne muscular dystrophy (DMD), Becker's disease, myotonic dystrophy, X-linked dilated cardiomyopathy, spinal muscular atrophy (SMA), or metaphyseal chondrodysplasia, Schmid type (MCDS). In some embodiments, the myopathy is a skeletal muscle atrophy. In some embodiments, the musculoskeletal disease (such as the skeletal muscle atrophy) is triggered by ageing, chronic diseases, stroke, malnutrition, bedrest, orthopedic injury, bone fracture, cachexia, starvation, heart failure, obstructive lung disease, renal failure, Acquired Immunodeficiency Syndrome (AIDS), sepsis, an immune disorder, a cancer, ALS, a burn injury, denervation, diabetes, muscle disuse, limb immobilization, mechanical unload, myositis, or a dystrophy.

In some embodiments, the disease or disorder is a genetic disorder, such as Down syndrome or MEHMO syndrome (Mental retardation, Epileptic seizures, Hypogenitalism, Microcephaly, and Obesity).

In some embodiments, a compound or salt thereof described herein or a composition described herein may be used in a method of treating musculoskeletal disease. In some embodiments, skeletal muscle mass, quality and/or strength are increased. In some embodiments, synthesis of muscle proteins is increased. In some embodiments, skeletal muscle fiber atrophy is inhibited.

In some embodiments, the disease or disorder is a vascular disease. In some embodiments, the vascular disease is atherosclerosis, abdominal aortic aneurism, carotid artery disease, deep vein thrombosis, Buerger's disease, chronic venous hypertension, vascular calcification, telangiectasia or lymphoedema.

In some embodiments, the disease or disorder is an ocular disease. In some embodiments, the ocular disease is glaucoma, age-related macular degeneration, inflammatory retinal disease, retinal vascular disease, diabetic retinopathy, uveitis, rosacea, Sjogren's syndrome, or neovascularization in proliferative retinopathy.

In some embodiments, provided herein is a method of modulating an ISR pathway. The compounds or salts thereof described herein and compositions described herein are believed to be effective for modulating an ISR pathway. In some embodiments, the method of modulating an ISR pathway comprises modulating the ISR pathway in a cell by administering or delivering to the cell a compound described herein, or a pharmaceutically acceptable salt thereof, or a pharmaceutical composition described herein. In some embodiments, the method of modulating an ISR pathway comprises modulating the ISR pathway in an individual by administering to the individual a compound described herein, or a pharmaceutically acceptable salt thereof, or a pharmaceutical composition described herein. Modulating of the ISR pathway can be determined by methods known in the art, such as western blot, immunohistochemistry, or reporter cell line assays.

In some embodiments, the modulation of the ISR pathway comprises binding eIF2B. In some embodiments, the modulation of the ISR pathway comprises increasing protein translation, increasing guanine nucleotide exchange factor (GEF) activity of eIF2B, delaying or preventing apoptosis in a cell, and/or modulating translation of one or more mRNAs comprising a 5′ untranslated region (5′UTR) comprising at least one upstream open reading frame (uORF).

In some embodiments, provided herein are methods of increasing protein production using a compound or salt described herein. The protein production is increased relative to the same condition without the compound or salt. Protein production can be increased either in vivo or in vitro. For example, protein production can be increased in vivo by administering the compound or salt to an individual. In some embodiments, protein production is increased in vitro using the compound or salt with a cell-free protein synthesis system (CFPS) or a cell-based protein expression system. The protein produced can be a heterologous protein (e.g., a recombinant protein) or a native protein. Heterologous protein production can be achieved using a recombinant nucleic acid encoding the protein. In some embodiments, the protein produced is an antibody or a fragment thereof. Other exemplary proteins can include, but are not limited to, enzymes, allergenic peptides or proteins (for example, for use as a vaccine), recombinant protein, cytokines, peptides, hormones, growth factors, erythropoietin (EPO), interferons, granulocyte-colony stimulating factor (G-CSF), anticoagulants, and clotting factors. The increase in protein production can be determined by methods known in the art, such as western blot or immunohistochemistry.

Cell-free protein synthesis (CFPS) systems are generally known, and include cellular machinery for protein expression in an in vitro environment. In some embodiments, the CFPS system includes a cellular extract (such as a eukaryotic cellular extract), which includes protein expression machinery. In some embodiment, the cellular machinery in the CFPS system comprises eukaryotic cellular machinery, such as eukaryotic initiation factor 2 (eIF2) and/or eukaryotic initiation factor 2B (eIF2B), or one or more subunits thereof.

In some embodiments, there is a cell-free protein synthesis (CFPS) system comprising eukaryotic initiation factor 2 (eIF2) and a nucleic acid encoding a protein with a compound or salt as described herein. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, growth factors, erythropoietin (EPO), interferons, granulocyte-colony stimulating factor (G-CSF), anticoagulants, and clotting factors. In some embodiments, the CFPS system comprises a cell extract comprising the eIF2. In some embodiments, the CFPS system further comprises eIF2B.

In some embodiments, there is a method of producing a protein, comprising contacting a cell-free protein synthesis (CFPS) system comprising eukaryotic initiation factor 2 (eIF2) and a nucleic acid encoding a protein with a compound or salt thereof as described herein. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, growth factors, erythropoietin (EPO), interferons, granulocyte-colony stimulating factor (G-CSF), anticoagulants, and clotting factors. In some embodiments, the CFPS system comprises a cell extract comprising the eIF2. In some embodiments, the CFPS system further comprises eIF2B. In some embodiments, the method comprises purifying the protein.

In some embodiments, there is a method of producing a protein, comprising contacting a eukaryotic cell comprising a nucleic acid encoding the protein with a compound or salt as described herein. In some embodiments, the method comprises culturing the cell in an in vitro culture medium comprising the compound or salt. In some embodiments, the nucleic acid encoding the protein is a recombinant nucleic acid. In some embodiments, the eukaryotic cell is a human embryonic kidney (HEK) cell, a Chinese hamster ovary (CHO) cell, or a HeLa cell. In some embodiments, the eukaryotic cell is a human embryonic kidney (HEK) cell or a Chinese hamster ovary (CHO) cell. In some embodiments, the eukaryotic cell is a human embryonic kidney (HEK) cell. In some embodiments, the eukaryotic cell is a Chinese hamster ovary (CHO) cell. In some embodiments, the eukaryotic cell is a HeLa cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a hybridoma cell, a human blood derived leukocyte, or a plant cell. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, growth factors, erythropoietin (EPO), interferons, granulocyte-colony stimulating factor (G-CSF), anticoagulants, and clotting factors. In some embodiments, the method comprises purifying the protein.

In some embodiments, there is a method of culturing a eukaryotic cell comprising a nucleic acid encoding a protein, comprising contacting the eukaryotic cell with an in vitro culture medium comprising a compound or salt as described herein. In some embodiments, the nucleic acid encoding the protein is a recombinant nucleic acid. In some embodiments, the eukaryotic cell is a human embryonic kidney (HEK) cell, a Chinese hamster ovary (CHO) cell, or a HeLa cell. In some embodiments, the eukaryotic cell is a human embryonic kidney (HEK) cell or a Chinese hamster ovary (CHO) cell. In some embodiments, the eukaryotic cell is a human embryonic kidney (HEK) cell. In some embodiments, the eukaryotic cell is a Chinese hamster ovary (CHO) cell. In some embodiments, the eukaryotic cell is a HeLa cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a hybridoma cell, a human blood derived leukocyte, or a plant cell. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, growth factors, erythropoietin (EPO), interferons, granulocyte-colony stimulating factor (G-CSF), anticoagulants, and clotting factors. In some embodiments, the method comprises purifying the protein.

In some embodiments, there is an in vitro cell culture medium, comprising the compound or salt described herein, and nutrients for cellular growth. In some embodiments, the culture medium comprises a eukaryotic cell comprising a nucleic acid encoding a protein. In some embodiments, the culture medium further comprises a compound for inducing protein expression. In some embodiments, the nucleic acid encoding the protein is a recombinant nucleic acid. In some embodiments, the protein is an antibody or a fragment thereof. Other exemplary proteins can include, but are not limited to, enzymes, allergenic peptides or proteins (for example, for use as a vaccine), recombinant protein, cytokines, peptides, hormones, growth factors, erythropoietin (EPO), interferons, granulocyte-colony stimulating factor (G-CSF), anticoagulants, and clotting factors. In some embodiments, the eukaryotic cell is a human embryonic kidney (HEK) cell, a Chinese hamster ovary (CHO) cell, or a HeLa cell. In some embodiments, the eukaryotic cell is a human embryonic kidney (HEK) cell or a Chinese hamster ovary (CHO) cell. In some embodiments, the eukaryotic cell is a human embryonic kidney (HEK) cell. In some embodiments, the eukaryotic cell is a Chinese hamster ovary (CHO) cell. In some embodiments, the eukaryotic cell is a HeLa cell. In other embodiments, the eukaryotic cell is a yeast cell (such as Saccharomyces cerevisiae or Pichia pastoris), a wheat germ cell, an insect cell, a rabbit reticulocyte, a cervical cancer cell (such as a HeLa cell), a baby hamster kidney cell (such as BHK21 cells), a murine myeloma cell (such as NSO or Sp2/0 cells), an HT-1080 cell, a PER.C6 cell, a hybridoma cell, a human blood derived leukocyte, or a plant cell.

In some embodiments, provided herein is a method of increasing protein translation in a cell or cell free expression system. In some embodiments, the cell was stressed prior to administration of the compound, salt thereof, or composition. In some embodiments, protein translation is increased by at least about 10%, 20%, 30%, 40%, 50%, 60%, 70%, 80%, 90%, 95%, 98%, 100%, 125%, 150%, 175%, 200%, 250%, or 300% or more. In some embodiments, protein translation is increased by about 10% to about 300% (such as about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, or about 250% to about 300%) In some embodiments, protein translation is increased as compared to prior to the administration of the compounds, salt thereof, or composition. In some embodiments, protein translation is increased as compared to an unstressed cell, a basal condition where cells are not subjected to a specific stress that activates the ISR. In some embodiments, protein translation is increased as compared to a stressed cell where ISR is active.

The compounds described herein may increase protein synthesis in a cell without full inhibition of ATF4 translation, under ISR-stressed or non-ISR stressed conditions. Despite ATF4 participation in various pathologies, the ATF4 protein is an important factor for restoring cellular homeostasis in stressed cells, for example during oxidative stress response, cholesterol metabolism, protein folding amino acid synthesis, and autophagy. Thus, for certain treatments, it may be preferable to limit or avoid ATF4 inhibition. In some embodiments, the compound is used to increase protein synthesis by about 10% or more, about 20% or more, about 30% or more, about 40% or more, about 50% or more, about 60% or more, about 70% or more, about 80% or more, about 90% or more, about 100% or more, about 125% or more, about 150% or more, about 175% or more, about 200% or more, about 250% or more, or about 300% or more wherein ATF4 protein expression is not substantially inhibited or is inhibited by about 75% or less, about 50% or less, about 40% or less, about 30% or less, about 20% or less, about 10% or less, or about 5% or less. In some embodiments the compound is used to increase protein synthesis by about 10% to about 1000% (such as about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 125% to about 150%, about 150% to about 175%, about 175% to about 200%, about 200% to about 250%, about 250% to about 300%, about 300% to about 350%, about 350% to about 400%, about 400% to about 450%, about 450% to about 500%, about 500% to about 600%, about 600% to about 700%, about 700% to about 800%, about 800% to about 900%, or about 900% to about 1000%), wherein ATF4 protein expression is not substantially inhibited or is inhibited by about 75% or less (such as about 50% or less, about 40% or less, about 30% or less, about 20% or less, about 10% or less, or about 5% or less).

In some embodiments, provided herein is a method of increasing protein translation in a cell. In some embodiments, the cell was stressed prior to administration of the compound, salt thereof, or composition. In some embodiments, protein translation is increased by at least about 10%, 20%, 30%, 40%, 50%, 60%, 70%, 80%, 90%, 95%, 98%, 100%, 125%, 150%, 175%, 200%, 250%, or 300% or more. In some embodiments, protein translation is increased as compared to prior to the administration of the compounds, salt thereof, or composition. In some embodiments, protein translation is increased as compared to an unstressed cell, a basal condition where cells are not subjected to a specific stress that activates the ISR. In some embodiments, protein translation is increased as compared to a stressed cell where ISR is active.

In some embodiments, provided herein is a method of increasing guanine nucleotide exchange factor (GEF) activity of eIF2B in cells. In some embodiments, provided herein is a method of delaying or preventing apoptosis in a cell. In some embodiments, provided herein is a method of inhibiting translation of one or more mRNAs comprising a 5′ untranslated region (5′UTR) that contains at least one upstream open reading frame (uORF), encoding proteins with translational preferences, including but not limited to ATF4, ATF2, ATF5, ATF3, FGF-21, CHOP, GADD34, BACE-1, C/EBPα, or MAP1LC3B. In some embodiments, the mRNA encodes ATF4, ATF3, FGF-21, BACE-1, GADD34, or CHOP. In some embodiments, the mRNA encodes ATF4, ATF2, ATF5, CHOP, GADD34, BACE-1, C/EBPα, or MAP1LC3B. In some embodiments, the mRNA encodes ATF4, BACE-1, GADD34, or CHOP. In some embodiments, the mRNA encodes ATF4.

In some embodiments, expression of ATF4, BACE-1, GADD34 or CHOP is inhibited. In some embodiments, expression of ATF4 is inhibited. In some embodiments, expression of Aβ is inhibited. ATF4 increases expression of, among others, GADD45A, CDKN1A, and EIF4EBP1, which encode DDIT-1, p21, and 4E-BP1, respectively. These proteins induce musculoskeletal disease (such as skeletal muscle atrophy), and can be modulated by inhibiting expression of ATF4. Accordingly, in some embodiments, expression of one or more of CDKN1A, GADD45A, or EIF4EBP1 is inhibited.

In some embodiments, the compound, salt thereof, or composition inhibits translation of one or more mRNAs comprising a 5′ untranslated region (5′UTR) comprising at least one upstream open reading frame (uORF) with an IC₅₀ of less than about 100 μM, such as less than about 75 μM, about 50 μM, about 25 μM, about 20 μM, about 10 μM, about 5 μM, about 1 μM, about 750 nM, 600 nM, 500 nM, 300 nM, 200 nM, 100 nM, 80 nM, 60 nM, 40 nM, 25 nM, or less. In some embodiments, the compound, salt thereof, or composition inhibits translation of one or more mRNAs comprising a 5′ untranslated region (5′UTR) comprising at least one upstream open reading frame (uORF) with an IC₅₀ between about 1 nM and 100 μM, such as between about 10 nM and 600 nM, 15 nM and 200 nM, or 20 nM and 180 nM.

In some embodiments, the compound, salt thereof, or composition inhibits expression of ATF4 with an IC₅₀ of less than about 100 μM, such as less than about 75 μM, about 50 μM, about 25 μM, about 20 μM, about 10 μM, about 5 μM, about 1 μM, about 750 nM, 600 nM, 500 nM, 300 nM, 200 nM, 100 nM, 80 nM, 60 nM, 40 nM, 25 nM, or less. In some embodiments, the compound, salt thereof, or composition inhibits expression of ATF4 with an IC₅₀ between about 1 nM and 100 μM, such as between about 2 nM and 800 nM, 10 nM and 600 nM, 15 nM and 200 nM, or 20 nM and 180 nM.

In some aspects, the half maximal inhibitory concentration (IC₅₀) is a measure of the effectiveness of a substance in inhibiting a specific biological or biochemical function. In some aspects, the IC₅₀ is a quantitative measure that indicates how much of an inhibitor is needed to inhibit a given biological process or component of a process such as an enzyme, cell, cell receptor or microorganism by half Methods of determining IC₅₀ in vitro and in vivo are known in the art.

In some embodiments, the individual is a mammal. In some embodiments, the individual is a primate, bovine, ovine, porcine, equine, canine, feline, rabbit, or rodent. In some embodiments, the individual is a human. In some embodiments, the individual has any of the diseases or disorders disclosed herein. In some embodiments, the individual is a risk for developing any of the diseases or disorders disclosed herein.

In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about any of 21, 18, 15, 12, 10, 8, 6, 5, 4, 3, 2, or 1 years old.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease mediated by an ISR pathway. In some embodiments, the manufacture of a medicament is for the prevention and/or treatment of a disorder or disease mediated by eIF2α or eIF2B. In some embodiments, the manufacture of a medicament is for the prevention and/or treatment of a disorder or disease mediated by phosphorylation of eIF2α and/or the GEF activity of eIF2B.

In some embodiments, there is a method for enhancing protein synthesis in a living organism, comprising administering to the living organism an effective amount of a compound or salt thereof as provided herein. In some embodiments, the living organism is selected from the group consisting of a cell suspension, a hairy root culture, moss protonema, an aquatic plant (including but not limited to duckweed and microalgae), and a terrestrial plant. In some embodiments, the living organism is a terrestrial plant. In some embodiments, the terrestrial plant is selected from soybean, sunflower, grain legume, rice, wheat germ, maize, tobacco, a cereal, and a lupin crop. In some embodiments, the terrestrial plant is tobacco.

In some embodiments, provided is a method for producing a protein in a living organism, comprising contacting the living organism with a compound described herein or a salt thereof (such as an agriculturally acceptable salt thereof), and wherein the protein is selected from the group consisting of a biopolymer, an industrial protein, an industrial enzyme, and a therapeutic protein. In some embodiments, the living organism is selected from the group consisting of a cell suspension, a hairy root culture, moss protonema, an aquatic plant (including but not limited to duckweed and microalgae), and a terrestrial plant. In some embodiments, the living organism is a terrestrial plant. In some embodiments, the terrestrial plant is tobacco. In some embodiments, the protein is an industrial protein selected from the group consisting of a hydrolase, a glycosidase (such as a cellulase, and a-amylase, a P-glucuronidase, and the likes), a protease (such as trypsin), and the likes. In some embodiments, the protein is a therapeutic protein selected from the group consisting of an antibody, a vaccine, a human growth-factor, a cytokine, and the likes.

In some embodiments, there is a method for accelerating growth of a plant, comprising administering to the plant an effective amount of a compound or salt thereof as provided herein. In some embodiments, the plant is an aquatic plant. In some embodiments, the plant is a terrestrial plant. In some embodiments, the terrestrial plant is selected from soybean, sunflower, grain legume, rice, wheat germ, maize, tobacco, a cereal, and a lupin crop. In some embodiments, the terrestrial plant is tobacco.

In some embodiments, there is a method for improving protein yield or quality in a plant, comprising administering to the plant an effective amount of a compound or salt thereof as provided herein. In some embodiments, the plant is an aquatic plant. In some embodiments, the plant is a terrestrial plant. In some embodiments, the terrestrial plant is selected from soybean, sunflower, grain legume, rice, wheat germ, maize, tobacco, a cereal, and a lupin crop. In some embodiments, the terrestrial plant is tobacco.

### Combinations

In certain aspects, a compound described herein is administered to an individual for treatment of a disease in combination with one or more additional pharmaceutical agents that can treat the disease. For example, in some embodiments, an effective amount of the compound is administered to an individual for the treatment of cancer in combination with one or more additional anticancer agents.

In some embodiments, activity of the additional pharmaceutical agent (such as additional anticancer agent) is inhibited by an activated ISR pathway. An ISR modulator, such as one of the compounds described herein, can inhibit the ISR pathway to enhance functionality of the additional pharmaceutical agent. By way of example, certain BRAF inhibitors (e.g., vemurafenib or dabrafenib) activate the ISR pathway in BRAF-mutated melanoma cells (e.g., BRAF with a V600F mutation) through the expression of ATF4. In some embodiments, there is a method of treating cancer comprising administering to an individual with cancer an effective amount of a compound described herein in combination with an effective amount of a BRAF inhibitor. In some embodiments, there is a method of treating a BRAF-mutated melanoma comprising administering to an individual with a BRAF-mutated melanoma an effective amount of a compound described herein in combination with an effective amount of a BRAF inhibitor. In some embodiments, there is a method of treating a BRAF-mutated melanoma comprising administering to an individual with a BRAF-mutated melanoma an effective amount of a compound described herein in combination with an effective amount of vemurafenib or dabrafenib.

As another example, certain anticancer agents (such as ubiquitin-proteasome pathway inhibitors (such as bortezomib), Cox-2 inhibitors (e.g., celecoxib), platinum-based antineoplastic drugs (e.g., cisplatin), anthracyclines (e.g. doxorubicin), or topoisomerase inhibitors (e.g., etoposide)) are used to treat cancer, but may have limited functionality against solid tumors. Resistance in certain solid tumors (e.g., breast cancers) has been associated with ATF4 stabilization and induction of autophagy. In some embodiments, an effective amount of an ISR inhibitor compound as described herein is administered to an individual with cancer to increase sensitivity to one or more anticancer agents.

In some embodiments, there is a method of treating a refractory cancer (such as a solid tumor) in an individual, comprising administering to the individual an effective amount of a compound described herein in combination with an effective amount of an anticancer agent. In some embodiments, there is a method of treating a refractory cancer (such as a solid tumor) in an individual, comprising administering to the individual an effective amount of a compound described herein in combination with an effective amount of an ubiquitin-proteasome pathway inhibitor (e.g., bortezomib), a Cox-2 inhibitor (e.g., celecoxib), a platinum-based antineoplastic drug (e.g., cisplatin), an anthracycline (e.g. doxorubicin), or a topoisomerase inhibitor (e.g., etoposide). In some embodiments, the refractory cancer is breast cancer. In some embodiments, the refractory cancer is melanoma.

In some embodiments, a compound described herein is used to treat cancer in combination with one or more anti-cancer agents, such as an anti-neoplastic agent, an immune checkpoint inhibitor, or any other suitable anti-cancer agent. Exemplary immune checkpoint inhibitors include anti-PD-1, anti-PD-L¹, anti GITR, anti-OX-40, anti-LAG3, anti-TIM-3, anti-41BB, anti-CTLA-4 antibodies. Exemplary anti-neoplastic agents can include, for example, anti-microtubule agents, platinum coordination complexes, alkylating agents, topoisomerase II inhibitors, topoisomerase I inhibitors, antimetabolites, antibiotic agents, hormones and hormonal analogs, signal transduction pathway inhibitors, non-receptor tyrosine kinase angiogenesis inhibitors, proteasome inhibitors, and inhibitors of cancer metabolism. Other anti-cancer agents can include one or more of an immuno-stimulant, an antibody or fragment thereof (e.g., an anti-CD20, anti-HER2, anti-CD52, or anti-VEGF antibody or fragment thereof), or an immunotoxin (e.g., an anti-CD33 antibody or fragment thereof, an anti-CD22 antibody or fragment thereof, a calicheamicin conjugate, or a pseudomonas exotoxin conjugate).

ATF4-mediated expression of CHOP has also been shown to regulate the function and accumulation of myeloid-derived suppressor cells (MDSCs) in tumors. MDSCs in tumors reduce the ability to prime T cell function and reduce antitumoral or anticancer responses. Certain immunotherapeutic agents (such as anti-PD-1, anti PD-L¹, anti-GITR, anti-OX-40, anti-LAG3, anti-TIM-3, anti-41BB, or anti-CTLA-4 antibodies) have been used to boost the immune response against cancer. ATF4-mediated expression of AXL has been associated with poor response to anti-PD1 therapy in melanoma. In some embodiments, an effective amount of an ISR modulator compound as described herein is administered to an individual with cancer to increase sensitivity to one or more immunotherapeutic agents. In some embodiments, there is a method of treating a refractory cancer (such as a melanoma) in an individual, comprising administering to the individual an effective amount of a compound described herein in combination with an effective amount of an immunotherapeutic agent (e.g. anti-PD-1, anti PD-L¹, anti-GITR, anti-OX-40, anti-LAG3, anti-TIM-3, anti-41BB, or anti-CTLA-4 antibodies). In some embodiments, the refractory cancer is melanoma.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or subunit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

Chromatography, recrystallization and other conventional separation procedures may also be used with intermediates or final products where it is desired to obtain a particular isomer of a compound or to otherwise purify a product of a reaction.

General methods of preparing compounds according to the present disclosure are depicted in the schemes below.

Compounds disclosed herein, such as compounds of formula (A-10) and (A-11), or pharmaceutically acceptable salts thereof, can be synthesized according to the general method described in the schemes below.

Provided herein are methods of synthesizing a compound of formula (A-10) or (A-11)

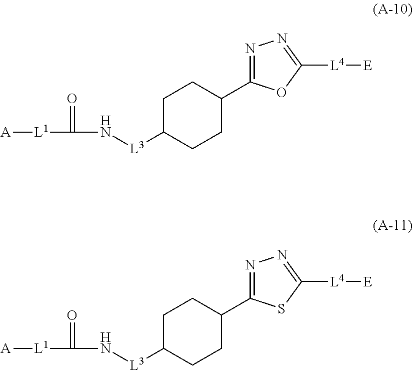

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L³ is a bond, —N(R^{L3})—, or —CH₂—;
   wherein R^{L3} is H, C₁-C₆ alkyl, or C₁-C₆ haloalkyl;
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (A-1), or a salt thereof, wherein L³ is as defined for the compound of formula (A-10) or the compound of formula (A-11), under suitable conditions to introduce a protecting group PG suitable to protect an amine group (such as, for example, a tert-butoxycarbonyl group), thereby giving a compound of formula (A-2), or a salt thereof, wherein L³ is as defined for the compound of formula (A-10) or the compound of formula (A-11). In some embodiments, the method further comprises reacting the compound of formula (A-2), or a salt thereof, under conditions suitable to give a compound of formula (A-3), or a salt thereof, wherein L³ is as defined for the compound of formula (A-10) or the compound of formula (A-11), and wherein PG is as defined for the compound of formula (A-2). In some embodiments, the method further comprises reacting the compound of formula (A-3), or a salt thereof, to give a compound of formula (A-4), or a salt thereof, wherein L³ is as defined for the compound of formula (A-10) or the compound of formula (A-11), and wherein PG is as defined for the compound of formula (A-2).

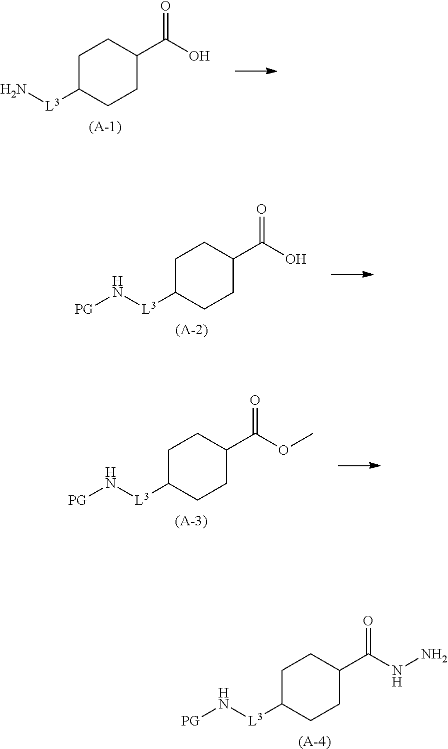

In some embodiments, the method comprises reacting a compound of formula (A-4), or a salt thereof, wherein L³ is as defined for the compound of formula (A-10) or the compound of formula (A-11), and wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (A-10) or the compound of formula (A-11), to give a compound of formula (A-5), or a salt thereof, wherein L³, L⁴, and E are as defined for the compound of formula (A-10) or the compound of formula (A-11).

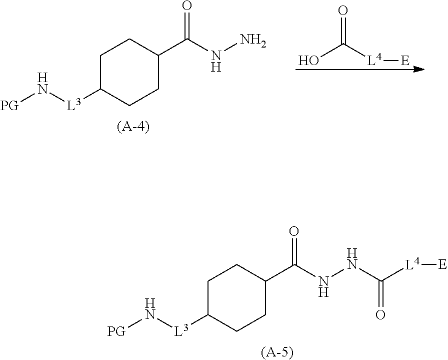

In some embodiments, the method comprises cyclizing a compound of formula (A-5), or a salt thereof, wherein L³, L⁴, and E are as defined for the compound of formula (A-10) or the compound of formula (A-11), to give a compound of formula (A-6), or a salt thereof, wherein L³, L⁴, and E are as defined for the compound of formula (A-10), or a compound formula (A-7), or a salt thereof, wherein L³, L⁴, and E are as defined for the compound of formula (A-11).

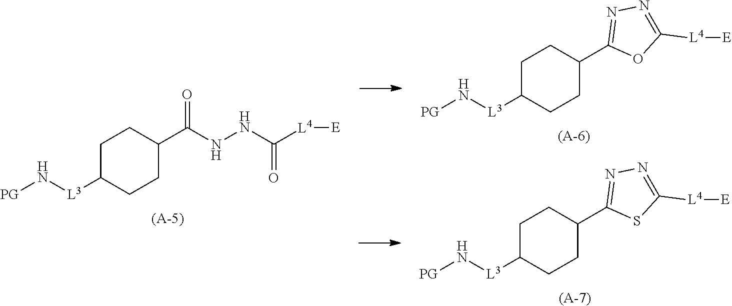

In some embodiments, the method comprises deprotecting a compound of formula (A-6), wherein L³, L⁴, and E are as defined for the compound of formula (A-10), or a salt thereof, to give a compound of formula (A-8), or a salt thereof, wherein L³, L⁴, and E are as defined for the compound of formula (A-10), or deprotecting a compound formula (A-7), or a salt thereof, wherein L³, L⁴, and E are as defined for the compound of formula (A-11), to give a compound of formula (A-9), or a salt thereof, wherein L³, L⁴, and E are as defined for the compound of formula (A-11).

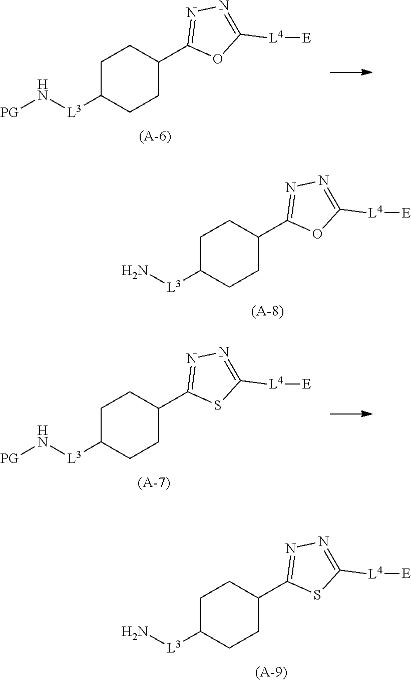

In some embodiments, the method comprises reacting a compound of formula (A-8), or a salt thereof, wherein L³, L⁴, and E are as defined for the compound of formula (A-10), to give the compound of formula (A-10), or a salt thereof, or reacting a compound formula (A-9), or a salt thereof, wherein L³, L⁴, and E are as defined for the compound of formula (A-11), to give a compound of formula (A-11), or a salt thereof.

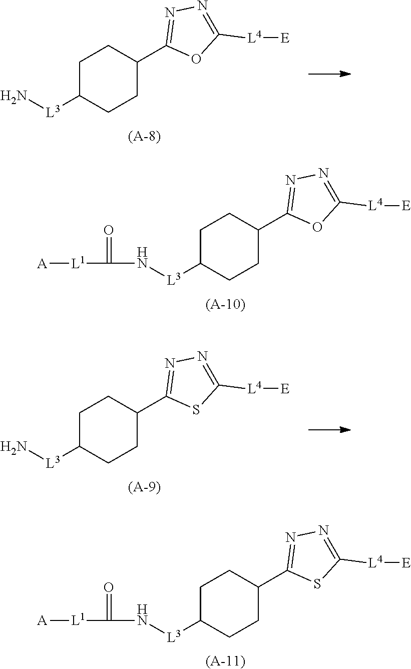

Provided herein are compounds of formula (A-10-a) and (A-11-a), or pharmaceutically acceptable salts thereof, synthesized according to the general method described in the schemes above.

In some embodiments, the methods can be performed with enantiopure materials. Provided herein are methods of synthesizing a compound of formula (A-10-a) or (A-11-a)

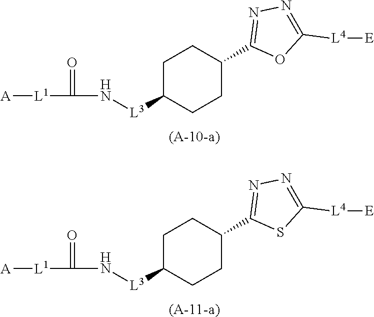

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L³ is a bond, —N(R^{L3})—, or —CH₂—;
   wherein R^{L3} is H, C₁-C₆ alkyl, or C₁-C₆ haloalkyl;
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (A-1-a), or a salt thereof, wherein L³ is as defined for the compound of formula (A-10-a) or the compound of formula (A-11-a), under suitable conditions to introduce a protecting group PG suitable to protect an amine group (such as, for example, a tert-butoxycarbonyl group), thereby giving a compound of formula (A-2-a), or a salt thereof, wherein L³ is as defined for the compound of formula (A-10-a) or the compound of formula (A-11-a). In some embodiments, the method further comprises reacting the compound of formula (A-2-a), or a salt thereof, under conditions suitable to give a compound of formula (A-3-a), or a salt thereof, wherein L³ is as defined for the compound of formula (A-10-a) or the compound of formula (A-11-a), and wherein PG is as defined for the compound of formula (A-2-a). In some embodiments, the method further comprises reacting the compound of formula (A-3-a), or a salt thereof, to give a compound of formula (A-4-a), or a salt thereof, wherein L³ is as defined for the compound of formula (A-10-a) or the compound of formula (A-11-a), and wherein PG is as defined for the compound of formula (A-2-a).

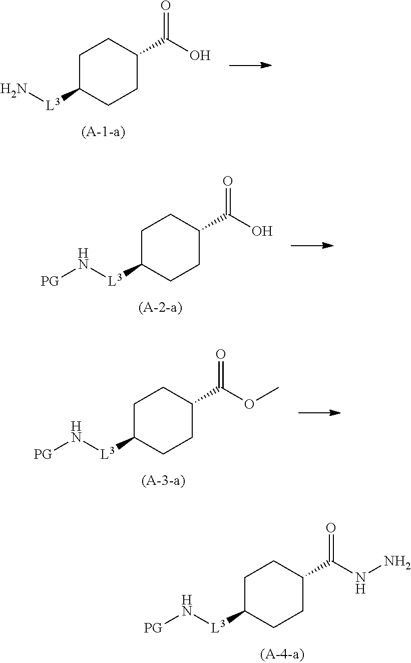

In some embodiments, the method comprises reacting a compound of formula (A-4-a), or a salt thereof, wherein L³ is as defined for the compound of formula (A-10-a) or the compound of formula (A-11-a), and wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (A-10-a) or the compound of formula (A-11-a), to give a compound of formula (A-5-a), or a salt thereof, wherein L³, L⁴, and E are as defined for the compound of formula (A-10-a) or the compound of formula (A-11-a), and wherein PG is a protecting group suitable to protect an amine group (such as, for example, a tert-butoxycarbonyl group).

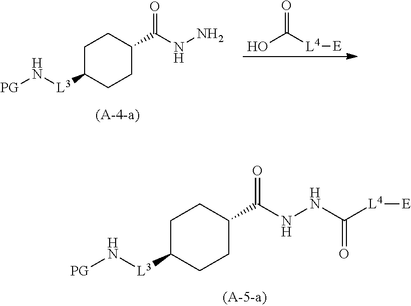

In some embodiments, the method comprises cyclizing a compound of formula (A-5-a), or a salt thereof, wherein L³, L⁴, and E are as defined for the compound of formula (A-10-a) or the compound of formula (A-11-a), and wherein PG is a protecting group suitable to protect an amine group (such as, for example, a tert-butoxycarbonyl group), to give a compound of formula (A-6-a), or a salt thereof, wherein L³, L⁴, and E are as defined for the compound of formula (A-10-a), or a compound formula (A-7-a), or a salt thereof, wherein L³, L⁴, and E are as defined for the compound of formula (A-11-a), and wherein PG is a protecting group suitable to protect an amine group (such as, for example, a tert-butoxycarbonyl group).

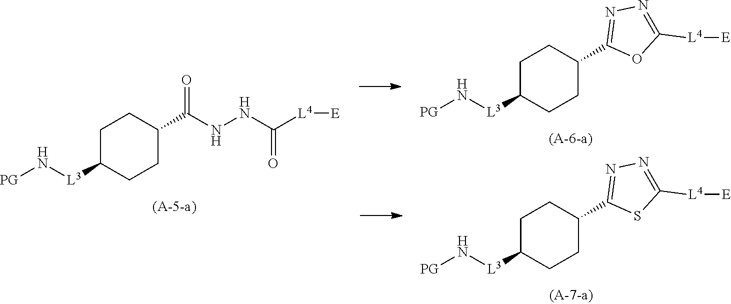

In some embodiments, the method comprises deprotecting a compound of formula (A-6-a), wherein L³, L⁴, and E are as defined for the compound of formula (A-10-a), and wherein PG is a protecting group suitable to protect an amine group (such as, for example, a tert-butoxycarbonyl group), or a salt thereof, to give a compound of formula (A-8-a), or a salt thereof, wherein L³, L⁴, and E are as defined for the compound of formula (A-10-a), or deprotecting a compound formula (A-7-a), or a salt thereof, wherein L³, L⁴, and E are as defined for the compound of formula (A-11-a), and wherein PG is a protecting group suitable to protect an amine group (such as, for example, a tert-butoxycarbonyl group), to give a compound of formula (A-9-a), or a salt thereof, wherein L³, L⁴, and E are as defined for the compound of formula (A-11-a).

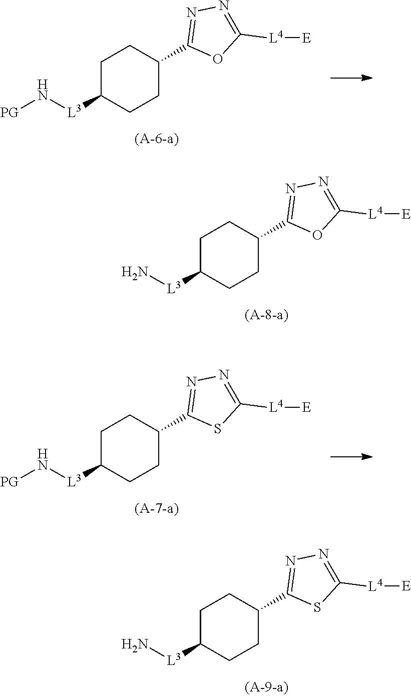

In some embodiments, the method comprises reacting a compound of formula (A-8-a), or a salt thereof, wherein L³, L⁴, and E are as defined for the compound of formula (A-10-a), to give the compound of formula (A-10-a), or a salt thereof, or reacting a compound formula (A-9-a), or a salt thereof, wherein L³, L⁴, and E are as defined for the compound of formula (A-11-a), to give a compound of formula (A-11-a), or a salt thereof.

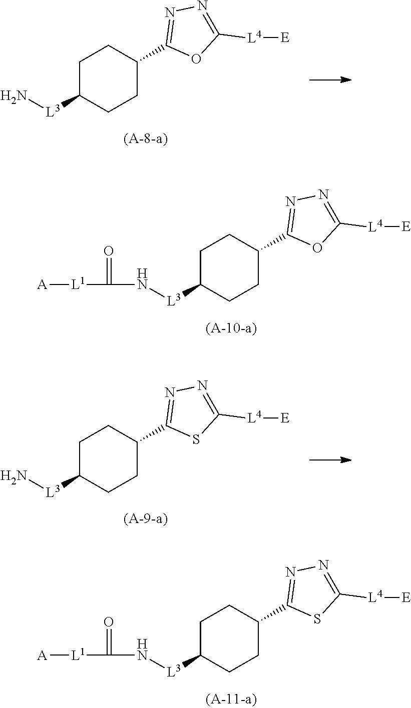

Provided herein are compounds of formula (A-10-a) and (A-11-a), or pharmaceutically acceptable salts thereof, synthesized according to the general method described in the schemes above.

In some embodiments, L³ is —(CH₂)ₙ—, wherein n is 0 (i.e. L³ is a bond) or n is 1 (i.e. L³ is —CH₂—. Compounds disclosed herein, such as compounds of formula (B-10) and (B-11), or pharmaceutically acceptable salts thereof, can be synthesized according to the general method described in the schemes below.

Provided herein are methods of synthesizing a compound of formula (B-10) or (B-11)

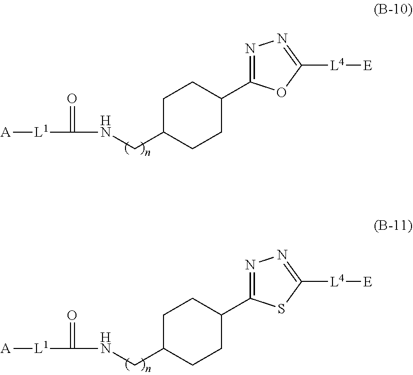

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
n is 0 or 1;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (B-1), or a salt thereof, wherein n is as defined for the compound of formula (B-10) or the compound of formula (B-11), under suitable conditions to introduce a protecting group PG suitable to protect an amine group (such as, for example, a tert-butoxycarbonyl group), thereby giving a compound of formula (B-2), or a salt thereof, wherein n is as defined for the compound of formula (B-10) or the compound of formula (B-11). In some embodiments, the method further comprises reacting the compound of formula (B-2), or a salt thereof, under conditions suitable to give a compound of formula (B-3), or a salt thereof, wherein n is as defined for the compound of formula (B-10) or the compound of formula (B-11), and wherein PG is as defined for the compound of formula (B-2). In some embodiments, the method further comprises reacting the compound of formula (B-3), or a salt thereof, to give a compound of formula (B-4), or a salt thereof, wherein n is as defined for the compound of formula (B-10) or the compound of formula (B-11), and wherein PG is as defined for the compound of formula (B-2).

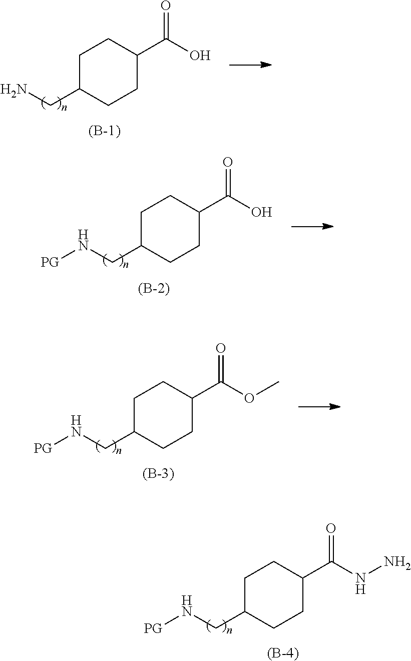

In some embodiments, the method comprises reacting a compound of formula (B-4), or a salt thereof, wherein n is as defined for the compound of formula (B-10) or the compound of formula (B-11), and wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (B-10) or the compound of formula (B-11), to give a compound of formula (B-5), or a salt thereof, wherein n, L⁴, and E are as defined for the compound of formula (B-10) or the compound of formula (B-11).

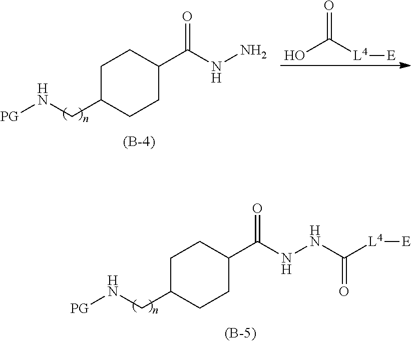

In some embodiments, the method comprises cyclizing a compound of formula (B-5), or a salt thereof, wherein n, L⁴, and E are as defined for the compound of formula (B-10) or the compound of formula (B-11), to give a compound of formula (B-6), or a salt thereof, wherein n, L4, and E are as defined for the compound of formula (B-10), or a compound formula (B-7), or a salt thereof, wherein n, L⁴, and E are as defined for the compound of formula (B-11).

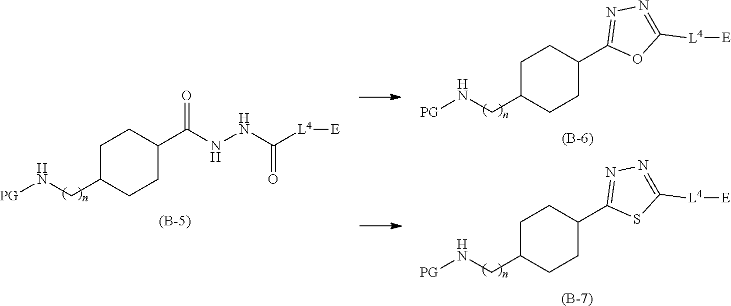

In some embodiments, the method comprises deprotecting a compound of formula (B-6), wherein n, L⁴, and E are as defined for the compound of formula (B-10), or a salt thereof, to give a compound of formula (B-8), or a salt thereof, wherein n, L⁴, and E are as defined for the compound of formula (B-10), or deprotecting a compound formula (B-7), or a salt thereof, wherein n, L⁴, and E are as defined for the compound of formula (B-11), to give a compound of formula (B-9), or a salt thereof, wherein n, L⁴, and E are as defined for the compound of formula (B-11).

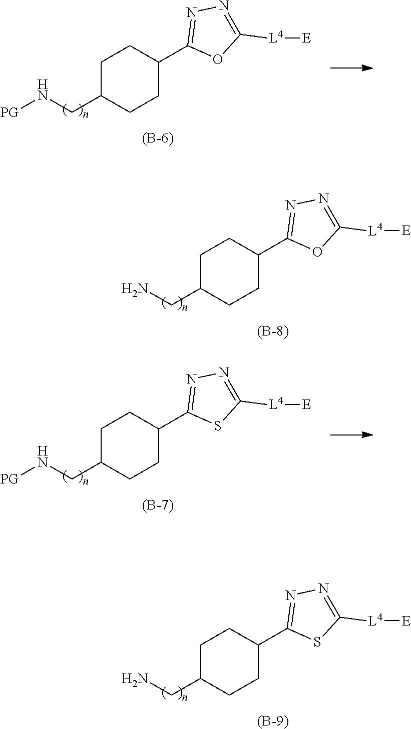

In some embodiments, the method comprises reacting a compound of formula (B-8), or a salt thereof, wherein n, L⁴, and E are as defined for the compound of formula (B-10), to give the compound of formula (B-10), or a salt thereof, or reacting a compound formula (B-9), or a salt thereof, wherein n, L⁴, and E are as defined for the compound of formula (B-11), to give a compound of formula (B-11), or a salt thereof.

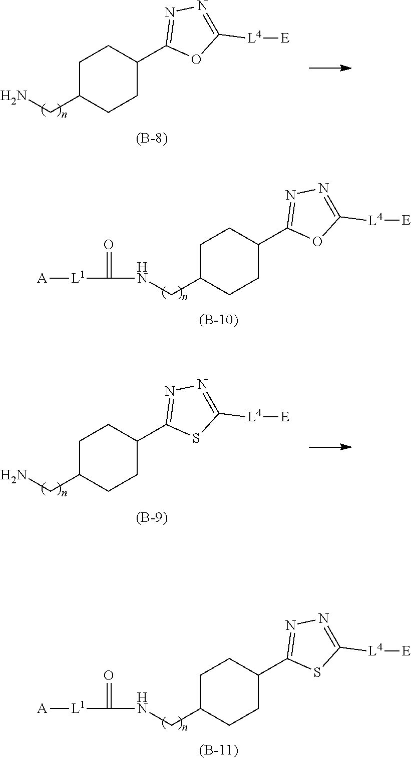

Provided herein are compounds of formula (B-10-a) and (B-11-a), or pharmaceutically acceptable salts thereof, synthesized according to the general method described in the schemes above.

In some embodiments, the methods can be performed with enantiopure materials. Provided herein are methods of synthesizing a compound of formula (B-10-a) or (B-11-a)

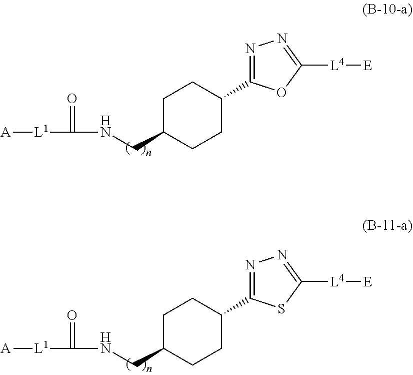

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
n is 0 or 1;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (B-1-a), or a salt thereof, wherein n is as defined for the compound of formula (B-10-a) or the compound of formula (B-11-a), under suitable conditions to introduce a protecting group PG suitable to protect an amine group (such as, for example, a tert-butoxycarbonyl group), thereby giving a compound of formula (B-2-a), or a salt thereof, wherein n is as defined for the compound of formula (B-10-a) or the compound of formula (B-11-a). In some embodiments, the method further comprises reacting the compound of formula (B-2-a), or a salt thereof, under conditions suitable to give a compound of formula (B-3-a), or a salt thereof, wherein n is as defined for the compound of formula (B-10-a) or the compound of formula (B-11-a), and wherein PG is as defined for the compound of formula (B-2-a). In some embodiments, the method further comprises reacting the compound of formula (B-3-a), or a salt thereof, to give a compound of formula (B-4-a), or a salt thereof, wherein n is as defined for the compound of formula (B-10-a) or the compound of formula (B-11-a), and wherein PG is as defined for the compound of formula (B-2-a).

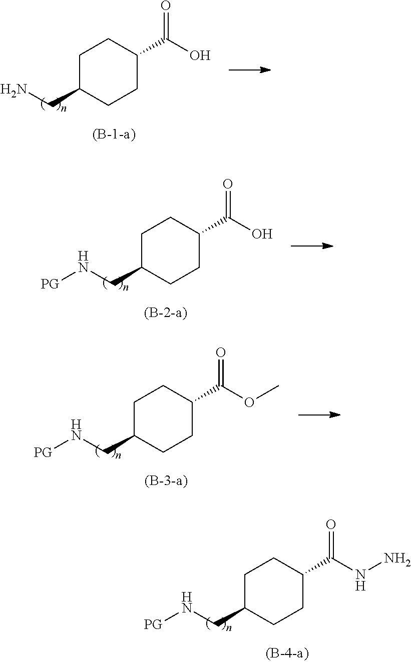

In some embodiments, the method comprises reacting a compound of formula (B-4-a), or a salt thereof, wherein n is as defined for the compound of formula (B-10-a) or the compound of formula (B-11-a), and wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (B-10-a) or the compound of formula (B-11-a), to give a compound of formula (B-5-a), or a salt thereof, wherein n, L⁴, and E are as defined for the compound of formula (B-10-a) or the compound of formula (B-11-a), and wherein PG is a protecting group suitable to protect an amine group (such as, for example, a tert-butoxycarbonyl group).

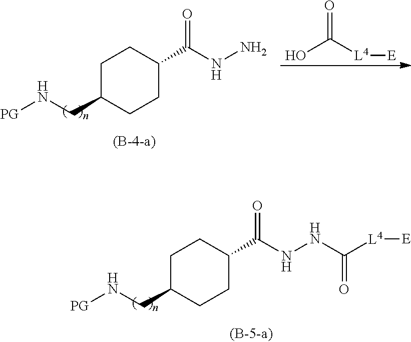

In some embodiments, the method comprises cyclizing a compound of formula (B-5-a), or a salt thereof, wherein n, L⁴, and E are as defined for the compound of formula (B-10-a) or the compound of formula (B-11-a), and wherein PG is a protecting group suitable to protect an amine group (such as, for example, a tert-butoxycarbonyl group), to give a compound of formula (B-6-a), or a salt thereof, wherein n, L⁴, and E are as defined for the compound of formula (B-10-a), or a compound formula (B-7-a), or a salt thereof, wherein n, L⁴, and E are as defined for the compound of formula (B-11-a), and wherein PG is a protecting group suitable to protect an amine group (such as, for example, a tert-butoxycarbonyl group).

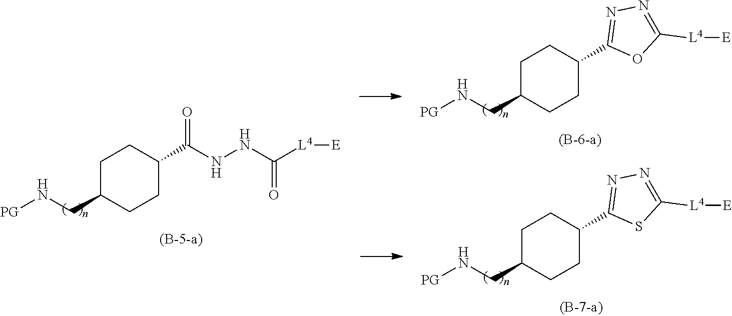

In some embodiments, the method comprises deprotecting a compound of formula (B-6-a), wherein n, L⁴, and E are as defined for the compound of formula (B-10-a), and wherein PG is a protecting group suitable to protect an amine group (such as, for example, a tert-butoxycarbonyl group), or a salt thereof, to give a compound of formula (B-8-a), or a salt thereof, wherein n, L⁴, and E are as defined for the compound of formula (B-10-a), or deprotecting a compound formula (B-7-a), or a salt thereof, wherein n, L⁴, and E are as defined for the compound of formula (B-11-a), and wherein PG is a protecting group suitable to protect an amine group (such as, for example, a tert-butoxycarbonyl group), to give a compound of formula (B-9-a), or a salt thereof, wherein n, L⁴, and E are as defined for the compound of formula (B-11-a).

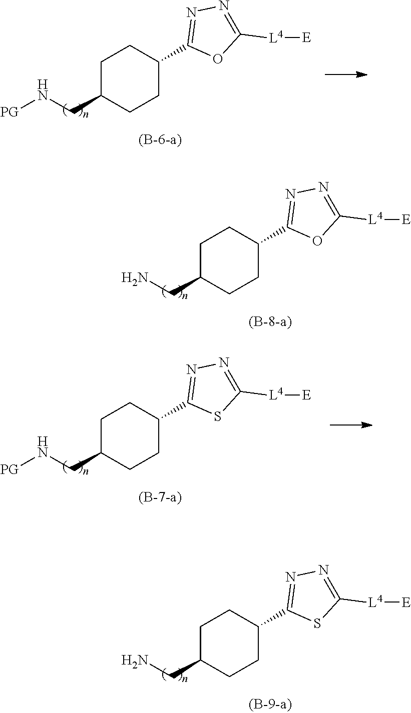

In some embodiments, the method comprises reacting a compound of formula (B-8-a), or a salt thereof, wherein n, L⁴, and E are as defined for the compound of formula (B-10-a), to give the compound of formula (B-10-a), or a salt thereof, or reacting a compound formula (B-9-a), or a salt thereof, wherein n, L⁴, and E are as defined for the compound of formula (B-11-a), to give a compound of formula (B-11-a), or a salt thereof.

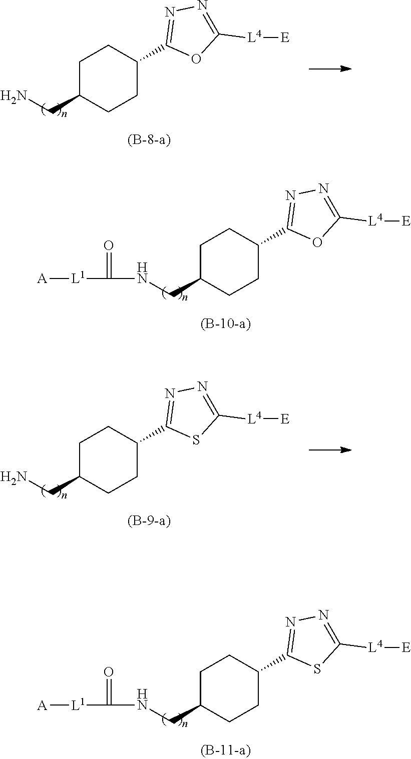

Provided herein are compounds of formula (B-10-a) and (B-11-a), or pharmaceutically acceptable salts thereof, synthesized according to the general method described in the schemes above.

In some embodiments, L³ is —CH₂—. Compounds disclosed herein, such as compounds of formula (C-10) and (C-11), or pharmaceutically acceptable salts thereof, can be synthesized according to the general method described in the schemes below.

Provided herein are methods of synthesizing a compound of formula (C-10) or (C-11)

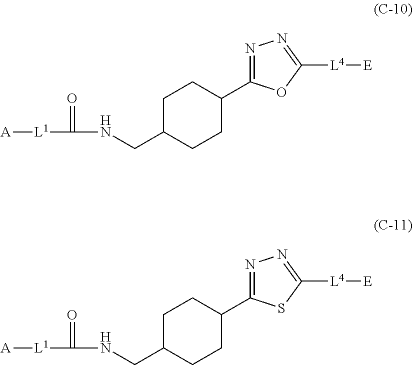

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (C-1), or a salt thereof, under suitable conditions to introduce a protecting group PG suitable to protect an amine group (such as, for example, a tert-butoxycarbonyl group), thereby giving a compound of formula (C-2), or a salt thereof. In some embodiments, the method further comprises reacting the compound of formula (C-2), or a salt thereof, under conditions suitable to give a compound of formula (C-3), or a salt thereof, wherein PG is as defined for the compound of formula (C-2). In some embodiments, the method further comprises reacting the compound of formula (C-3), or a salt thereof, to give a compound of formula (C-4), or a salt thereof, wherein PG is as defined for the compound of formula (C-2).

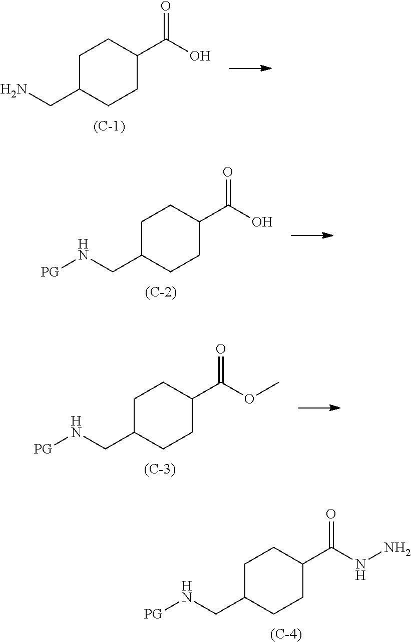

In some embodiments, the method comprises reacting a compound of formula (C-4), or a salt thereof, wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (C-10) or the compound of formula (C-11), to give a compound of formula (C-5), or a salt thereof, wherein L⁴ and E are as defined for the compound of formula (C-10) or the compound of formula (C-11).

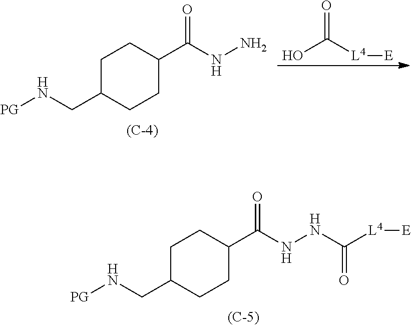

In some embodiments, the method comprises cyclizing a compound of formula (C-5), or a salt thereof, wherein L⁴ and E are as defined for the compound of formula (C-10) or the compound of formula (C-11), to give a compound of formula (C-6), or a salt thereof, wherein L⁴ and E are as defined for the compound of formula (C-10), or a compound formula (C-7), or a salt thereof, wherein L⁴ and E are as defined for the compound of formula (C-11).

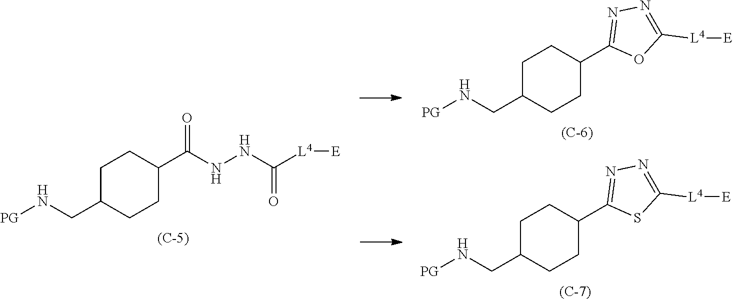

In some embodiments, the method comprises deprotecting a compound of formula (C-6), wherein L⁴ and E are as defined for the compound of formula (C-10), or a salt thereof, to give a compound of formula (C-8), or a salt thereof, wherein L⁴ and E are as defined for the compound of formula (C-10), or deprotecting a compound formula (C-7), or a salt thereof, wherein L⁴ and E are as defined for the compound of formula (C-11), to give a compound of formula (C-9), or a salt thereof, wherein L⁴ and E are as defined for the compound of formula (C-11).

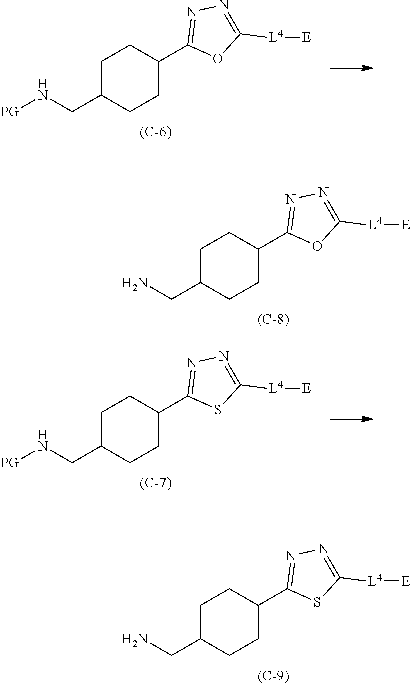

In some embodiments, the method comprises reacting a compound of formula (C-8), or a salt thereof, wherein L⁴ and E are as defined for the compound of formula (C-10), to give the compound of formula (C-10), or a salt thereof, or reacting a compound formula (C-9), or a salt thereof, wherein L⁴ and E are as defined for the compound of formula (C-11), to give a compound of formula (C-11), or a salt thereof.

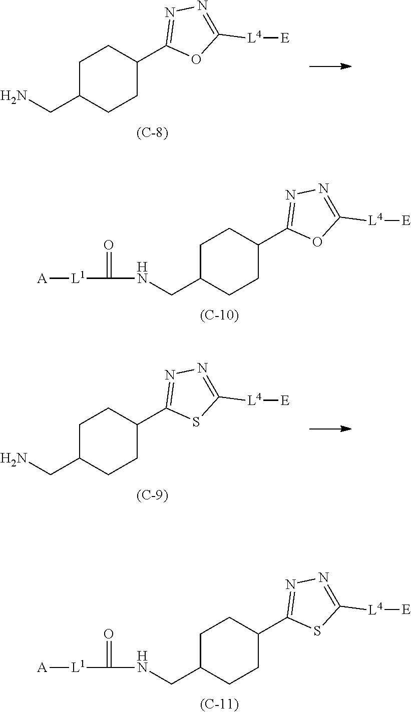

Provided herein are compounds of formula (C-10-a) and (C-11-a), or pharmaceutically acceptable salts thereof, synthesized according to the general method described in the schemes above.

In some embodiments, the methods can be performed with enantiopure materials. Provided herein are methods of synthesizing a compound of formula (C-10-a) or (C-11-a)

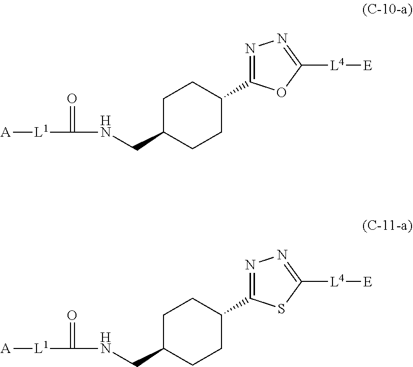

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁—C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (C-1-a), or a salt thereof, under suitable conditions to introduce a protecting group PG suitable to protect an amine group (such as, for example, a tert-butoxycarbonyl group), thereby giving a compound of formula (C-2-a), or a salt thereof. In some embodiments, the method further comprises reacting the compound of formula (C-2-a), or a salt thereof, under conditions suitable to give a compound of formula (C-3-a), or a salt thereof, wherein PG is as defined for the compound of formula (C-2-a). In some embodiments, the method further comprises reacting the compound of formula (C-3-a), or a salt thereof, to give a compound of formula (C-4-a), or a salt thereof, wherein PG is as defined for the compound of formula (C-2-a).

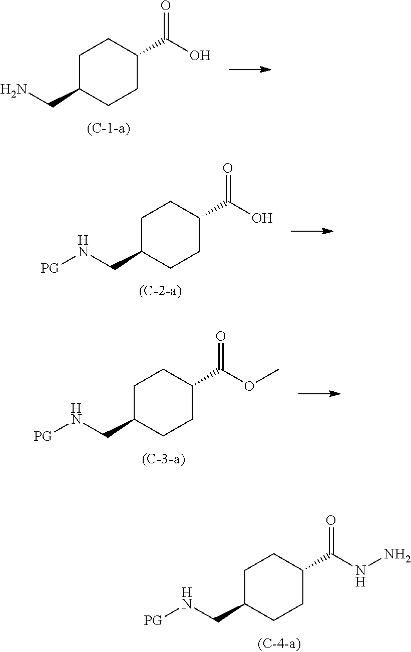

In some embodiments, the method comprises reacting a compound of formula (C-4-a), or a salt thereof, wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (C-10-a) or the compound of formula (C-11-a), to give a compound of formula (C-5-a), or a salt thereof, wherein L⁴ and E are as defined for the compound of formula (C-10-a) or the compound of formula (C-11-a), and wherein PG is a protecting group suitable to protect an amine group (such as, for example, a tert-butoxycarbonyl group).

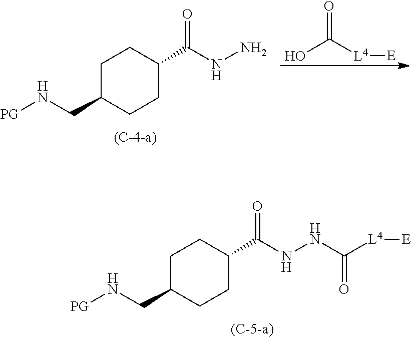

In some embodiments, the method comprises cyclizing a compound of formula (C-5-a), or a salt thereof, wherein L⁴ and E are as defined for the compound of formula (C-10-a) or the compound of formula (C-11-a), and wherein PG is a protecting group suitable to protect an amine group (such as, for example, a tert-butoxycarbonyl group), to give a compound of formula (C-6-a), or a salt thereof, wherein L⁴ and E are as defined for the compound of formula (C-10-a), or a compound formula (C-7-a), or a salt thereof, wherein L⁴ and E are as defined for the compound of formula (C-11-a), and wherein PG is a protecting group suitable to protect an amine group (such as, for example, a tert-butoxycarbonyl group).

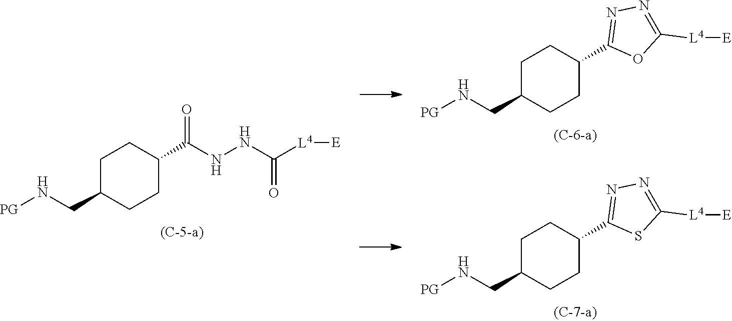

In some embodiments, the method comprises deprotecting a compound of formula (C-6-a), wherein L⁴ and E are as defined for the compound of formula (C-10-a), and wherein PG is a protecting group suitable to protect an amine group (such as, for example, a tert-butoxycarbonyl group), or a salt thereof, to give a compound of formula (C-8-a), or a salt thereof, wherein L⁴ and E are as defined for the compound of formula (C-10-a), or deprotecting a compound formula (C-7-a), or a salt thereof, wherein L⁴ and E are as defined for the compound of formula (C-11-a), and wherein PG is a protecting group suitable to protect an amine group (such as, for example, a tert-butoxycarbonyl group), to give a compound of formula (C-9-a), or a salt thereof, wherein L⁴ and E are as defined for the compound of formula (C-11-a).

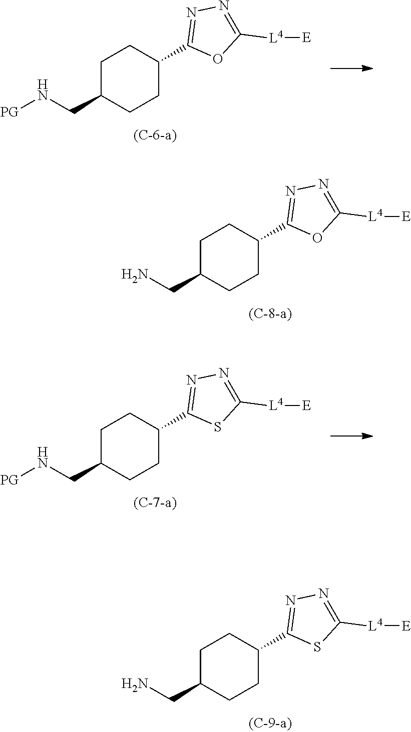

In some embodiments, the method comprises reacting a compound of formula (C-8-a), or a salt thereof, wherein L⁴ and E are as defined for the compound of formula (C-10-a), to give the compound of formula (C-10-a), or a salt thereof, or reacting a compound formula (C-9-a), or a salt thereof, wherein L⁴ and E are as defined for the compound of formula (C-11-a), to give a compound of formula (C-11-a), or a salt thereof.

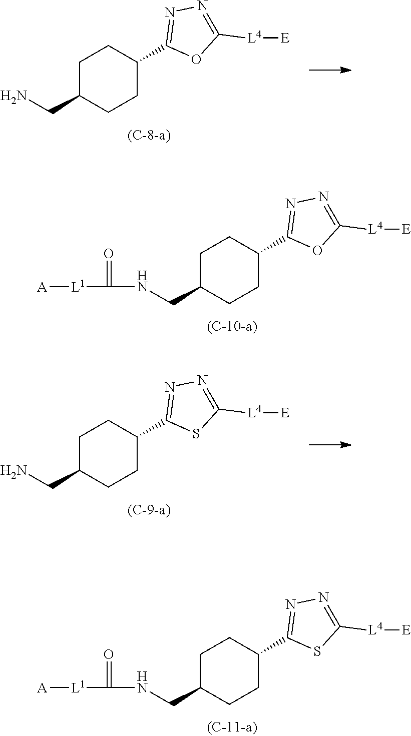

Provided herein are compounds of formula (C-10-a) and (C-11-a), or pharmaceutically acceptable salts thereof, synthesized according to the general method described in the schemes above.

In some embodiments, L³ is a bond. Compounds disclosed herein, such as compounds of formula (D-10) and (D-11), or pharmaceutically acceptable salts thereof, can be synthesized according to the general method described in the schemes below.

Provided herein are methods of synthesizing a compound of formula (D-10) or (D-11)

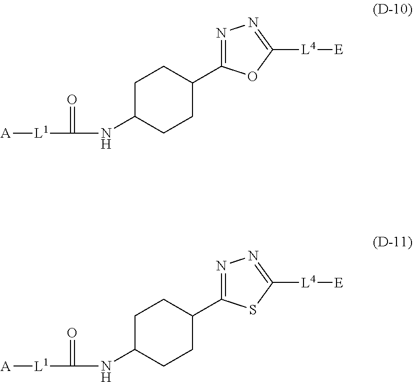

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (D-1), or a salt thereof, under suitable conditions to introduce a protecting group PG suitable to protect an amine group (such as, for example, a tert-butoxycarbonyl group), thereby giving a compound of formula (D-2), or a salt thereof. In some embodiments, the method further comprises reacting the compound of formula (D-2), or a salt thereof, under conditions suitable to give a compound of formula (D-3), or a salt thereof, wherein PG is as defined for the compound of formula (D-2). In some embodiments, the method further comprises reacting the compound of formula (D-3), or a salt thereof, to give a compound of formula (D-4), or a salt thereof, wherein PG is as defined for the compound of formula (D-2).

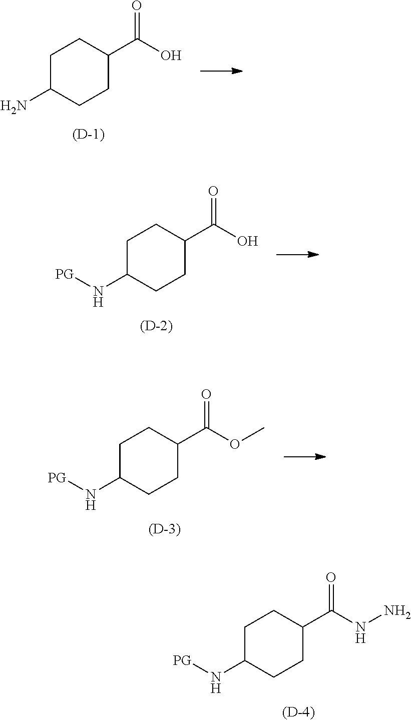

In some embodiments, the method comprises reacting a compound of formula (D-4), or a salt thereof, wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (D-10) or the compound of formula (D-11), to give a compound of formula (D-5), or a salt thereof, wherein L⁴ and E are as defined for the compound of formula (D-10) or the compound of formula (D-11).

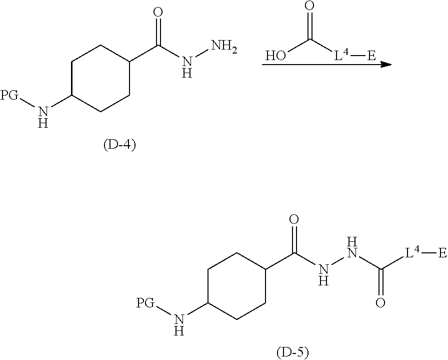

In some embodiments, the method comprises cyclizing a compound of formula (D-5), or a salt thereof, wherein L⁴ and E are as defined for the compound of formula (D-10) or the compound of formula (D-11), to give a compound of formula (D-6), or a salt thereof, wherein L⁴ and E are as defined for the compound of formula (D-10), or a compound formula (D-7), or a salt thereof, wherein L⁴ and E are as defined for the compound of formula (D-11).

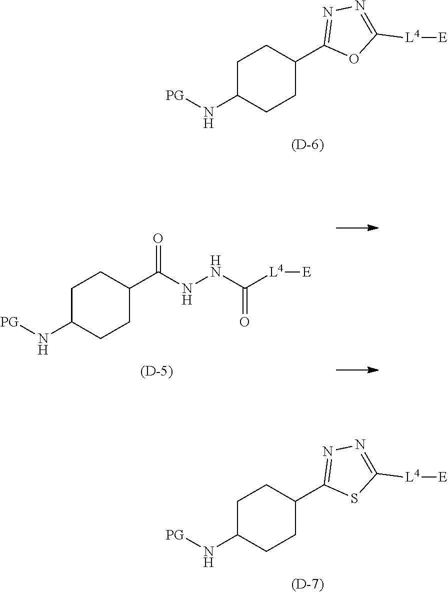

In some embodiments, the method comprises deprotecting a compound of formula (D-6), wherein L⁴ and E are as defined for the compound of formula (D-10), or a salt thereof, to give a compound of formula (D-8), or a salt thereof, wherein L⁴ and E are as defined for the compound of formula (D-10), or deprotecting a compound formula (D-7), or a salt thereof, wherein L⁴ and E are as defined for the compound of formula (D-11), to give a compound of formula (D-9), or a salt thereof, wherein L⁴ and E are as defined for the compound of formula (D-11).

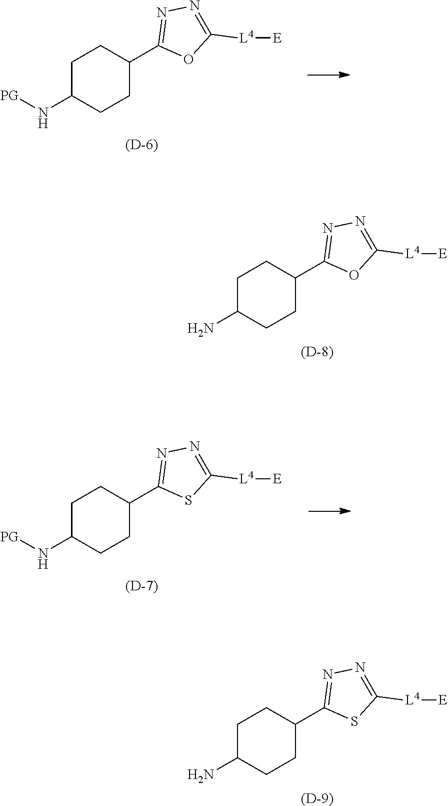

In some embodiments, the method comprises reacting a compound of formula D-8), or a salt thereof, wherein L⁴ and E are as defined for the compound of formula (D-10), to give the compound of formula (D-10), or a salt thereof, or reacting a compound formula (D-9), or a salt thereof, wherein L⁴ and E are as defined for the compound of formula (D-11), to give a compound of formula (D-11), or a salt thereof.

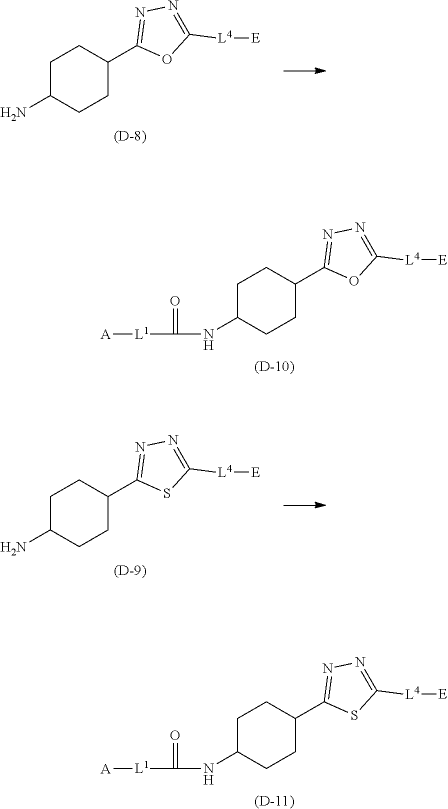

Provided herein are compounds of formula (D-10-a) and (D-11-a), or pharmaceutically acceptable salts thereof, synthesized according to the general method described in the schemes above.

In some embodiments, the methods can be performed with enantiopure materials. Provided herein are methods of synthesizing a compound of formula (D-10-a) or (D-11-a)

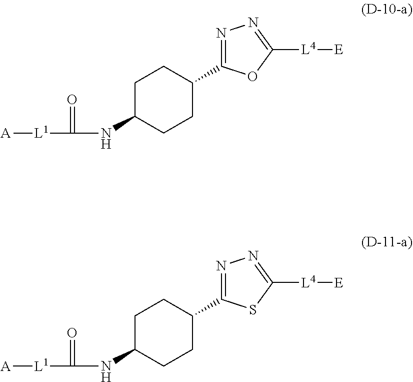

wherein A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁—C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (D-1-a), or a salt thereof, under suitable conditions to introduce a protecting group PG suitable to protect an amine group (such as, for example, a tert-butoxycarbonyl group), thereby giving a compound of formula (D-2-a), or a salt thereof. In some embodiments, the method further comprises reacting the compound of formula (D-2-a), or a salt thereof, under conditions suitable to give a compound of formula (D-3-a), or a salt thereof, wherein PG is as defined for the compound of formula (D-2-a). In some embodiments, the method further comprises reacting the compound of formula (D-3-a), or a salt thereof, to give a compound of formula (D-4-a), or a salt thereof, wherein PG is as defined for the compound of formula (D-2-a).

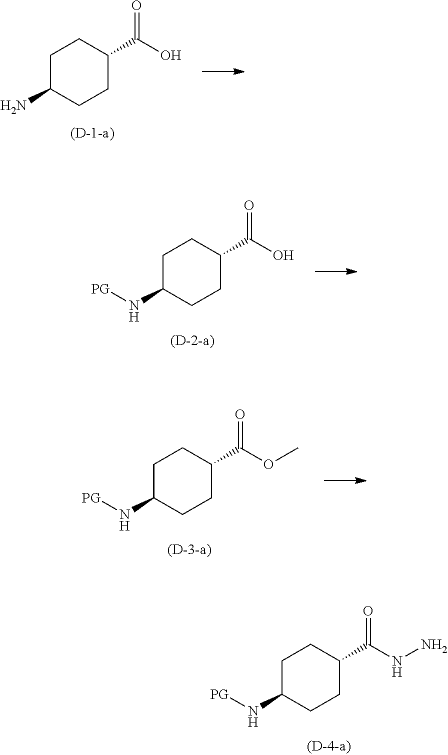

In some embodiments, the method comprises reacting a compound of formula (D-4-a), or a salt thereof, wherein PG is a protecting group suitable to protect an amine group (such as, for example, a tert-butoxycarbonyl group), with a carboxylic acid of formula E-L⁴-COOH, or a derivative of said carboxylic acid, wherein L⁴ and E are as defined for the compound of formula (D-10-a) or the compound of formula (D-11-a), to give a compound of formula (D-5-a), or a salt thereof, wherein L⁴ and E are as defined for the compound of formula (D-10-a) or the compound of formula (D-11-a), and wherein PG is a protecting group suitable to protect an amine group (such as, for example, a tert-butoxycarbonyl group).

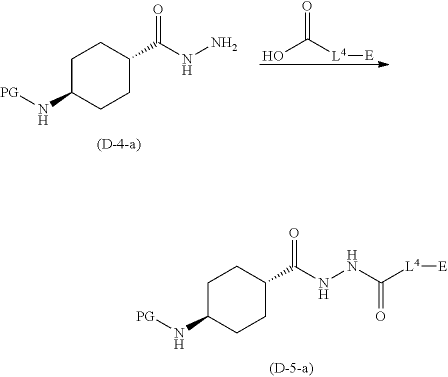

In some embodiments, the method comprises cyclizing a compound of formula (D-5-a), or a salt thereof, wherein L⁴ and E are as defined for the compound of formula (D-10-a) or the compound of formula (D-11-a), and wherein PG is a protecting group suitable to protect an amine group (such as, for example, a tert-butoxycarbonyl group), to give a compound of formula (D-6-a), or a salt thereof, wherein L⁴ and E are as defined for the compound of formula (D-10-a), or a compound formula (D-7-a), or a salt thereof, wherein L⁴ and E are as defined for the compound of formula (D-11-a), and wherein PG is a protecting group suitable to protect an amine group (such as, for example, a tert-butoxycarbonyl group).

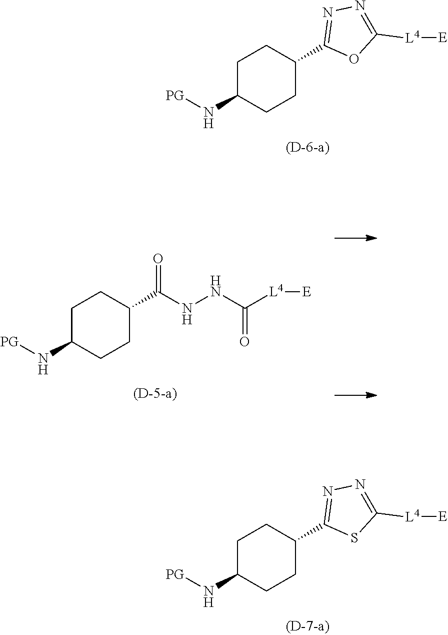

In some embodiments, the method comprises deprotecting a compound of formula (D-6-a), wherein L⁴ and E are as defined for the compound of formula (D-10-a), and wherein PG is a protecting group suitable to protect an amine group (such as, for example, a tert-butoxycarbonyl group), or a salt thereof, to give a compound of formula (D-8-a), or a salt thereof, wherein L⁴ and E are as defined for the compound of formula (D-10-a), or deprotecting a compound formula (D-7-a), or a salt thereof, wherein L⁴ and E are as defined for the compound of formula (D-11-a), and wherein PG is a protecting group suitable to protect an amine group (such as, for example, a tert-butoxycarbonyl group), to give a compound of formula (D-9-a), or a salt thereof, wherein L⁴ and E are as defined for the compound of formula (D-11-a).

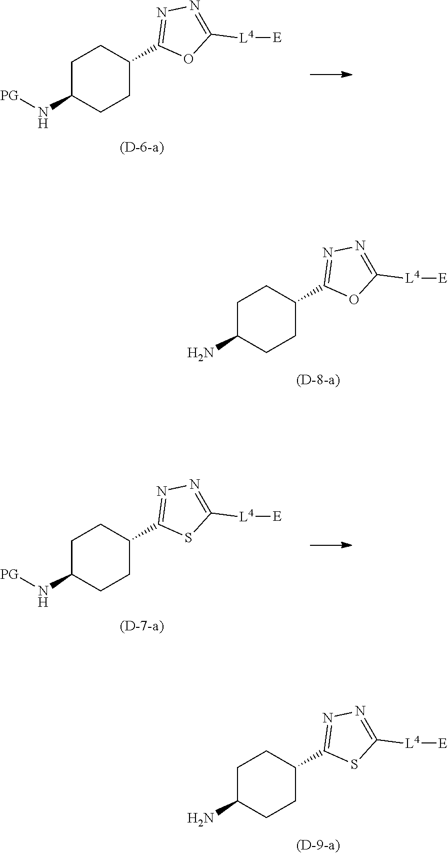

In some embodiments, the method comprises reacting a compound of formula (D-8-a), or a salt thereof, wherein L⁴ and E are as defined for the compound of formula (D-10-a), to give the compound of formula (D-10-a), or a salt thereof, or reacting a compound formula (D-9-a), or a salt thereof, wherein L⁴ and E are as defined for the compound of formula (D-11-a), to give a compound of formula (D-11-a), or a salt thereof.

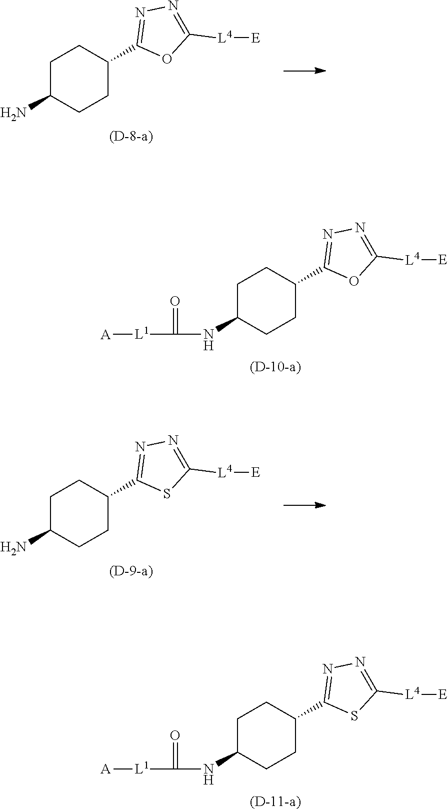

Provided herein are compounds of formula (D-10-a) and (D-11-a), or pharmaceutically acceptable salts thereof, synthesized according to the general method described in the schemes above.

Compounds disclosed herein, such as compounds of formula (E-5), or pharmaceutically acceptable salts thereof, can be synthesized according to the general method described in the schemes below.

Provided herein are methods of synthesizing a compound of formula (E-5)

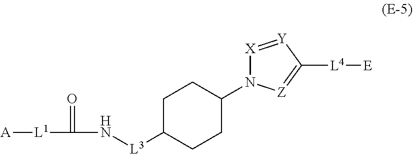

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L³ is a bond, —N(R^{L3})—, or —CH₂—; wherein R^{L3} is H, C₁-C₆ alkyl, or C₁-C₆ haloalkyl;
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (E-1), or a salt thereof, wherein L³ is as defined for the compound of formula (E-5), and wherein PG is a protecting group suitable to protect an amine group (such as, for example, a tert-butoxycarbonyl group), under suitable conditions to convert the hydroxyl group of the compound of formula (E-1) into a leaving group (such as, for example, a methanesulfonate group, a trifluoromethanesulfonate group, or a 4-methylbenzenesulfonate group), thereby giving a compound of formula (E-2), or a salt thereof, wherein L³ is as defined for the compound of formula (E-5), and wherein PG is as defined for the compound of formula (E-1). In some embodiments, the method further comprises reacting the compound of formula (E-2), or a salt thereof, under conditions suitable to give a compound of formula (E-3), or a salt thereof, wherein L³, X, Y, Z, L⁴, and E, are as defined for the compound of formula (E-5), and wherein PG is as defined for the compound of formula (E-1).

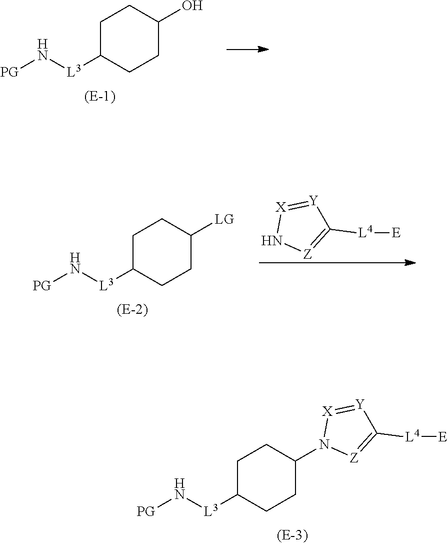

In some embodiments, the method comprises deprotecting a compound of formula (E-3), wherein L³, X, Y, Z, L⁴, and E, are as defined for the compound of formula (E-5), and wherein PG is a protecting group suitable to protect an amine group (such as, for example, a tert-butoxycarbonyl group), to give a compound of formula (E-4), or a salt thereof, wherein L³, X, Y, Z, L⁴, and E, are as defined for the compound of formula (E-5).

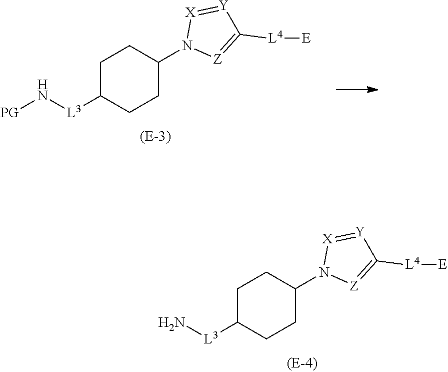

In some embodiments, the method comprises reacting a compound of formula (E-4), or a salt thereof, wherein L³, X, Y, Z, L⁴, and E, are as defined for the compound of formula (E-5), to give the compound of formula (E-5), or a salt thereof.

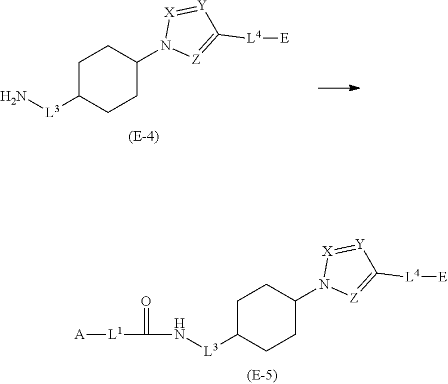

Provided herein are compounds of formula (E-5), or pharmaceutically acceptable salts thereof, synthesized according to the general method described in the schemes above.

In some embodiments, the methods can be performed with enantiopure materials. Compounds disclosed herein, such as compounds of formula (E-5-a), or pharmaceutically acceptable salts thereof, can be synthesized according to the general method described in the schemes below.

Provided herein are methods of synthesizing a compound of formula (E-5-a)

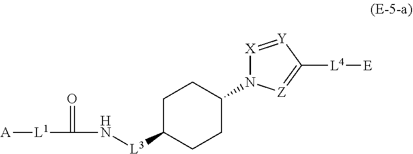

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L³ is a bond, —N(R^{L3})—, or —CH₂—; wherein R^{L3} is H, C₁-C₆ alkyl, or C₁-C₆ haloalkyl;
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁—C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (E-1-a), or a salt thereof, wherein L³ is as defined for the compound of formula (E-5-a), and wherein PG is a protecting group suitable to protect an amine group (such as, for example, a tert-butoxycarbonyl group), under suitable conditions to convert the hydroxyl group of the compound of formula (E-1-a) into a leaving group (such as, for example, a methanesulfonate group, a trifluoromethanesulfonate group, or a 4-methylbenzenesulfonate group), thereby giving a compound of formula (E-2-a), or a salt thereof, wherein L³ is as defined for the compound of formula (E-5-a), and wherein PG is as defined for the compound of formula (E-1-a). In some embodiments, the method further comprises reacting the compound of formula (E-2-a), or a salt thereof, under conditions suitable to give a compound of formula (E-3-a), or a salt thereof, wherein L³, X, Y, Z, L⁴, and E, are as defined for the compound of formula (E-5-a), and wherein PG is as defined for the compound of formula (E-1-a).

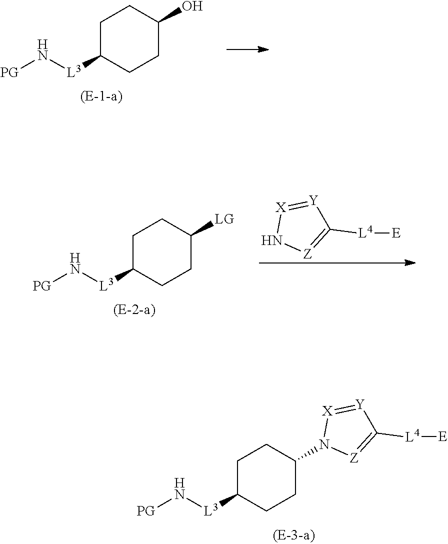

In some embodiments, the method comprises deprotecting a compound of formula (E-3-a), wherein L³, X, Y, Z, L⁴, and E, are as defined for the compound of formula (E-5-a), and wherein PG is as defined for the compound of formula (E-1-a), and wherein PG is a protecting group suitable to protect an amine group (such as, for example, a tert-butoxycarbonyl group), to give a compound of formula (E-4-a), or a salt thereof, wherein L³, X, Y, Z, L⁴, and E, are as defined for the compound of formula (E-5-a).

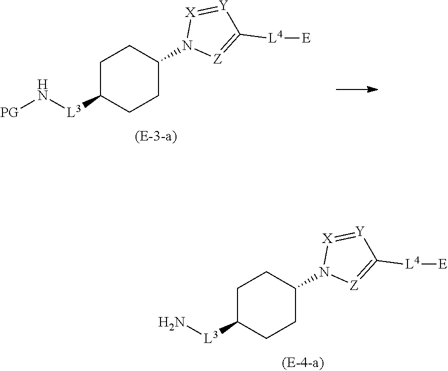

In some embodiments, the method comprises reacting a compound of formula (E-4-a), or a salt thereof, wherein L³, X, Y, Z, L⁴, and E, are as defined for the compound of formula (E-5-a), to give the compound of formula (E-5-a), or a salt thereof.

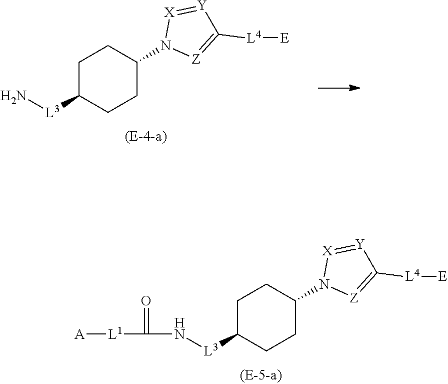

Provided herein are compounds of formula (E-5-a), or pharmaceutically acceptable salts thereof, synthesized according to the general method described in the schemes above.

In some embodiments, L³ is —(CH₂)ₙ—, wherein n is 0 (i.e. L³ is a bond) or n is 1 (i.e. L³ is —CH₂—. Compounds disclosed herein, such as compounds of formula (F-5), or pharmaceutically acceptable salts thereof, can be synthesized according to the general method described in the schemes below.

Provided herein are methods of synthesizing a compound of formula (F-5)

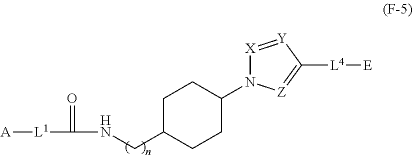

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
n is 0 or 1;
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (F-1), or a salt thereof, wherein n is as defined for the compound of formula (F-5), and wherein PG is a protecting group suitable to protect an amine group (such as, for example, a tert-butoxycarbonyl group), under suitable conditions to convert the hydroxyl group of the compound of formula (F-1) into a leaving group (such as, for example, a methanesulfonate group, a trifluoromethanesulfonate group, or a 4-methylbenzenesulfonate group), thereby giving a compound of formula (F-2), or a salt thereof, wherein PG is as defined for the compound of formula (F-1). In some embodiments, the method further comprises reacting the compound of formula (F-2), or a salt thereof, under conditions suitable to give a compound of formula (F-3), or a salt thereof, wherein n, X, Y, Z, and L4, are as defined for the compound of formula (F-5), and wherein PG is as defined for the compound of formula (F-1).

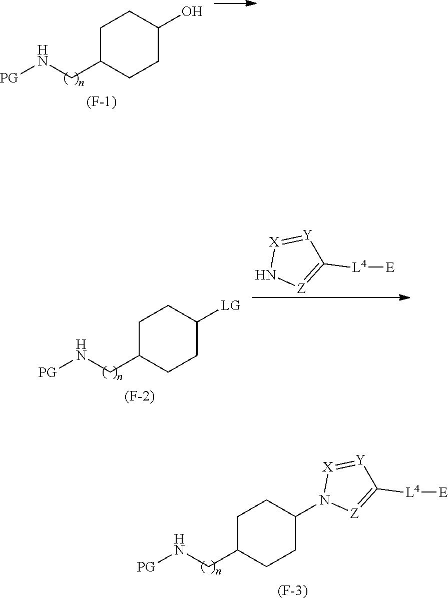

In some embodiments, the method comprises deprotecting a compound of formula (F-3), wherein n, X, Y, Z, and L⁴, are as defined for the compound of formula (F-5), and wherein PG is a protecting group suitable to protect an amine group (such as, for example, a tert-butoxycarbonyl group), to give a compound of formula (F-4), or a salt thereof, wherein n, X, Y, Z, and L⁴, are as defined for the compound of formula (F-5).

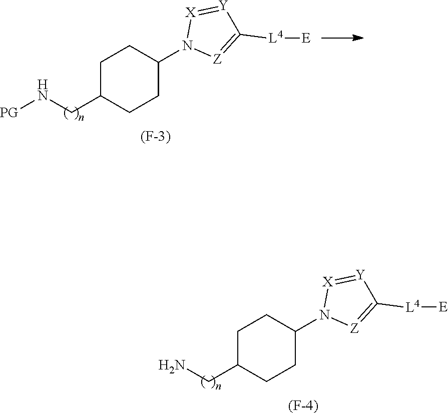

In some embodiments, the method comprises reacting a compound of formula (F-4), or a salt thereof, wherein n, X, Y, Z, and L⁴, are as defined for the compound of formula (F-5), to give the compound of formula (F-5), or a salt thereof.

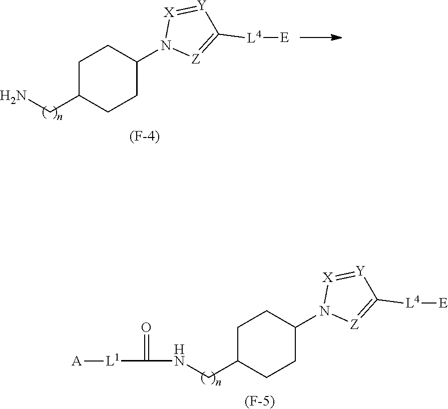

Provided herein are compounds of formula (F-5), or pharmaceutically acceptable salts thereof, synthesized according to the general method described in the schemes above.

In some embodiments, the methods can be performed with enantiopure materials. Compounds disclosed herein, such as compounds of formula (F-5-a), or pharmaceutically acceptable salts thereof, can be synthesized according to the general method described in the schemes below.

Provided herein are methods of synthesizing a compound of formula (F-5-a)

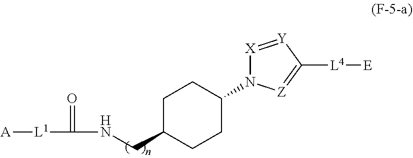

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
n is 0 or 1;
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁—C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (F-1-a), or a salt thereof, wherein n is as defined for the compound of formula (F-5-a), and wherein PG is a protecting group suitable to protect an amine group (such as, for example, a tert-butoxycarbonyl group), under suitable conditions to convert the hydroxyl group of the compound of formula (F-1-a) into a leaving group (such as, for example, a methanesulfonate group, a trifluoromethanesulfonate group, or a 4-methylbenzenesulfonate group), thereby giving a compound of formula (F-2-a), or a salt thereof, wherein n is as defined for the compound of formula (F-5-a), and wherein PG is as defined for the compound of formula (F-1-a). In some embodiments, the method further comprises reacting the compound of formula (F-2-a), or a salt thereof, under conditions suitable to give a compound of formula (F-3-a), or a salt thereof, wherein n, X, Y, Z, and L⁴, are as defined for the compound of formula (F-5-a), and wherein PG is as defined for the compound of formula (F-1-a).

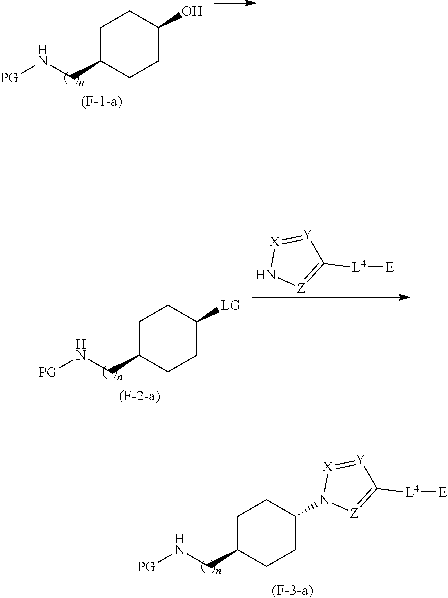

In some embodiments, the method comprises deprotecting a compound of formula (F-3-a), wherein n, X, Y, Z, and L⁴, are as defined for the compound of formula (F-5-a), and wherein PG is a protecting group suitable to protect an amine group (such as, for example, a tert-butoxycarbonyl group), to give a compound of formula (F-4-a), or a salt thereof, wherein n, X, Y, Z, and L⁴, are as defined for the compound of formula (F-5-a).

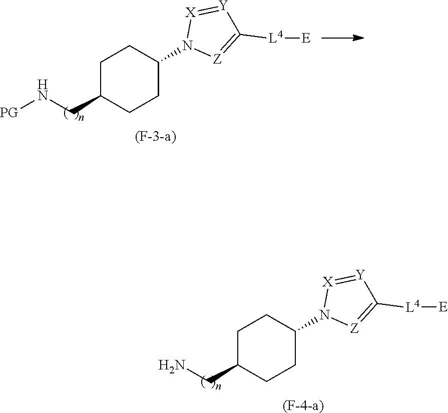

In some embodiments, the method comprises reacting a compound of formula (F-4-a), or a salt thereof, wherein n, X, Y, Z, and L⁴, are as defined for the compound of formula (F-5-a), to give the compound of formula (F-5-a), or a salt thereof.

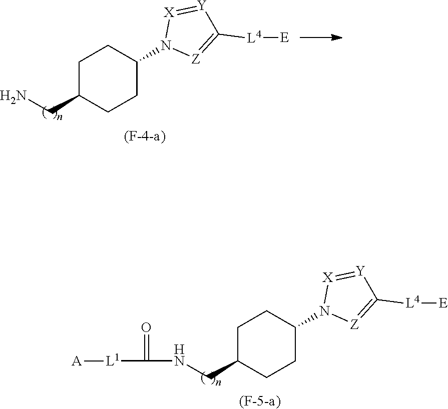

Provided herein are compounds of formula (F-5-a), or pharmaceutically acceptable salts thereof, synthesized according to the general method described in the schemes above.

In some embodiments, L³ is —CH₂—. Compounds disclosed herein, such as compounds of formula (G-5), or pharmaceutically acceptable salts thereof, can be synthesized according to the general method described in the schemes below.

Provided herein are methods of synthesizing a compound of formula (G-5)

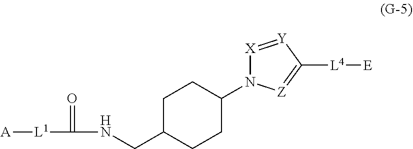

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (G-1), or a salt thereof, wherein PG is a protecting group suitable to protect an amine group (such as, for example, a tert-butoxycarbonyl group), under suitable conditions to convert the hydroxyl group of the compound of formula (G-1) into a leaving group (such as, for example, a methanesulfonate group, a trifluoromethanesulfonate group, or a 4-methylbenzenesulfonate group), thereby giving a compound of formula (G-2), or a salt thereof, wherein PG is as defined for the compound of formula (G-1). In some embodiments, the method further comprises reacting the compound of formula (G-2), or a salt thereof, under conditions suitable to give a compound of formula (G-3), or a salt thereof, wherein X, Y, Z, and L⁴, are as defined for the compound of formula (G-5), and wherein PG is as defined for the compound of formula (G-1).

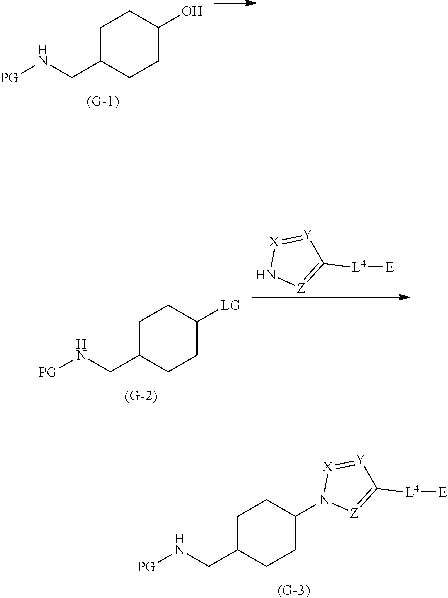

In some embodiments, the method comprises deprotecting a compound of formula (G-3), wherein X, Y, Z, and L⁴, are as defined for the compound of formula (G-5), and wherein PG is a protecting group suitable to protect an amine group (such as, for example, a tert-butoxycarbonyl group), to give a compound of formula (G-4), or a salt thereof, wherein X, Y, Z, and L⁴, are as defined for the compound of formula (G-5).

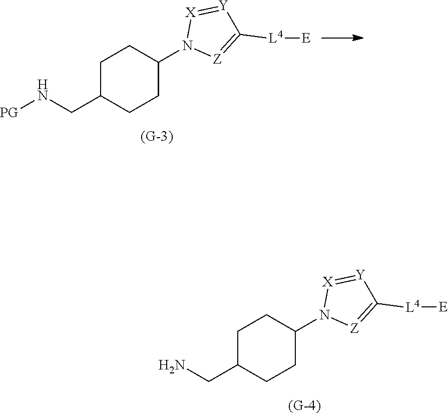

In some embodiments, the method comprises reacting a compound of formula (G-4), or a salt thereof, wherein X, Y, Z, and L⁴, are as defined for the compound of formula (G-5), to give the compound of formula (G-5), or a salt thereof.

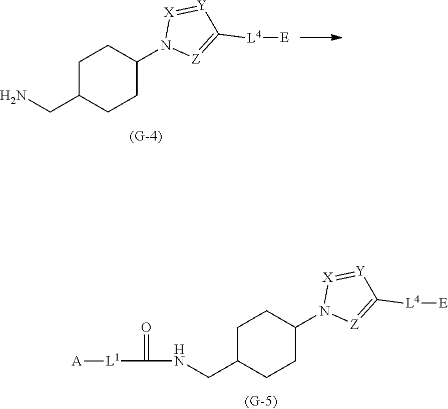

Provided herein are compounds of formula (G-5), or pharmaceutically acceptable salts thereof, synthesized according to the general method described in the schemes above.

In some embodiments, the methods can be performed with enantiopure materials. Compounds disclosed herein, such as compounds of formula (G-5-a), or pharmaceutically acceptable salts thereof, can be synthesized according to the general method described in the schemes below.

Provided herein are methods of synthesizing a compound of formula (G-5-a)

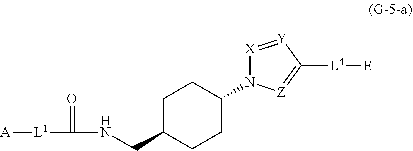

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (G-1-a), or a salt thereof, wherein PG is a protecting group suitable to protect an amine group (such as, for example, a tert-butoxycarbonyl group), under suitable conditions to convert the hydroxyl group of the compound of formula (G-1-a) into a leaving group (such as, for example, a methanesulfonate group, a trifluoromethanesulfonate group, or a 4-methylbenzenesulfonate group), thereby giving a compound of formula (G-2-a), or a salt thereof, wherein PG is as defined for the compound of formula (G-1-a). In some embodiments, the method further comprises reacting the compound of formula (G-2-a), or a salt thereof, under conditions suitable to give a compound of formula (G-3-a), or a salt thereof, wherein X, Y, Z, and L⁴, are as defined for the compound of formula (G-5-a), and wherein PG is as defined for the compound of formula (G-1-a).

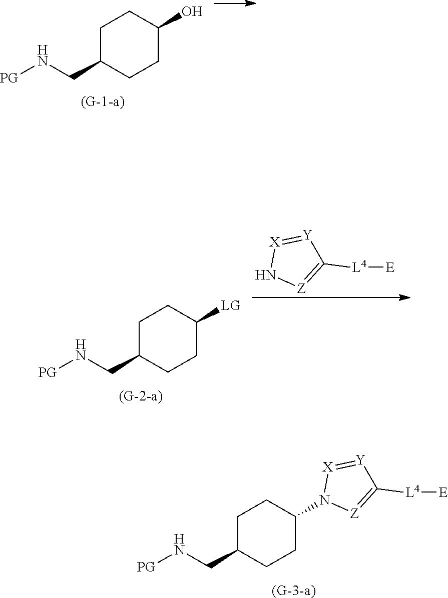

In some embodiments, the method comprises deprotecting a compound of formula (G-3-a), wherein X, Y, Z, and L⁴, are as defined for the compound of formula (G-5-a), and wherein PG is a protecting group suitable to protect an amine group (such as, for example, a tert-butoxycarbonyl group), to give a compound of formula (G-4-a), or a salt thereof, wherein X, Y, Z, and L⁴, are as defined for the compound of formula (G-5-a).

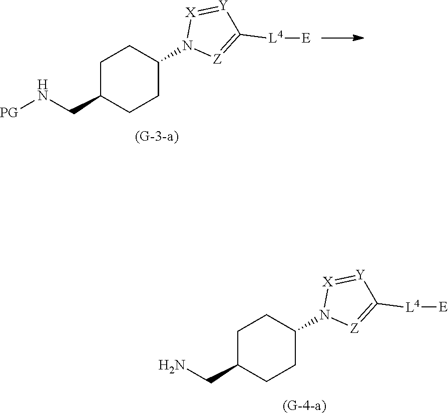

In some embodiments, the method comprises reacting a compound of formula (G-4-a), or a salt thereof, wherein X, Y, Z, and L⁴, are as defined for the compound of formula (G-5-a), to give the compound of formula (G-5-a), or a salt thereof.

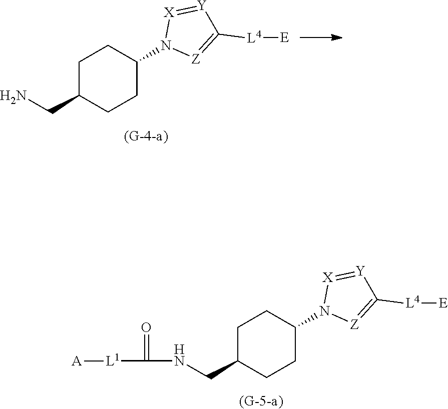

Provided herein are compounds of formula (G-5-a), or pharmaceutically acceptable salts thereof, synthesized according to the general method described in the schemes above.

In some embodiments, L³ is a bond. Compounds disclosed herein, such as compounds of formula (H-5), or pharmaceutically acceptable salts thereof, can be synthesized according to the general method described in the schemes below.

Provided herein are methods of synthesizing a compound of formula (H-5)

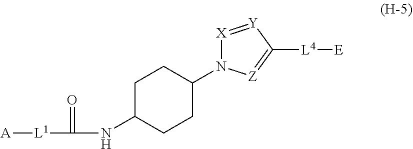

wherein X, Y, and Z, are, independently from each other, selected from the group consisting of NH, NR^{D}, CH, and CR^{D}, provided that:
   at least one of X, Y, and Z, is CH or CR^{D};
   when Z is NH or NR^{D}, then X is CH or CR^{D}; and
   when X is NH or NR^{D}, and Y is NH or NR^{D}, then Z is CH or CR^{D};
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁—C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (H-1), or a salt thereof, wherein PG is a protecting group suitable to protect an amine group (such as, for example, a tert-butoxycarbonyl group), under suitable conditions to convert the hydroxyl group of the compound of formula (H-1) into a leaving group (such as, for example, a methanesulfonate group, a trifluoromethanesulfonate group, or a 4-methylbenzenesulfonate group), thereby giving a compound of formula (H-2), or a salt thereof, wherein PG is as defined for the compound of formula (H-1). In some embodiments, the method further comprises reacting the compound of formula (H-2), or a salt thereof, under conditions suitable to give a compound of formula (H-3), or a salt thereof, wherein X, Y, Z, and L⁴, are as defined for the compound of formula (H-5), and wherein PG is as defined for the compound of formula (H-1).

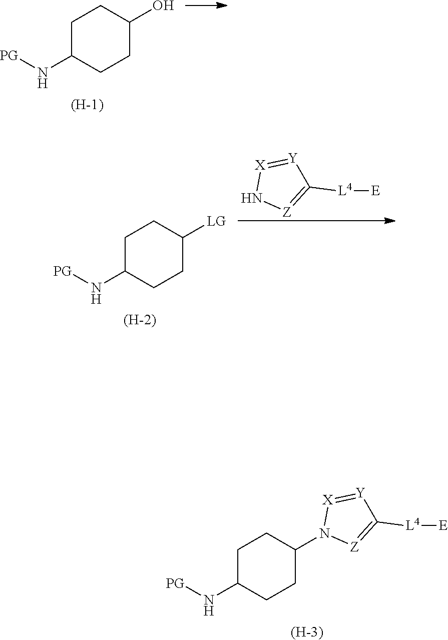

In some embodiments, the method comprises deprotecting a compound of formula (H-3), wherein X, Y, Z, and L⁴, are as defined for the compound of formula (H-5), and wherein PG is a protecting group suitable to protect an amine group (such as, for example, a tert-butoxycarbonyl group), to give a compound of formula (H-4), or a salt thereof, wherein X, Y, Z, and L⁴, are as defined for the compound of formula (H-5).

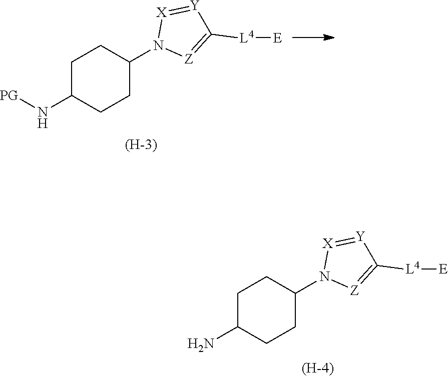

In some embodiments, the method comprises reacting a compound of formula (H-4), or a salt thereof, wherein X, Y, Z, and L⁴, are as defined for the compound of formula (H-5), to give the compound of formula (H-5), or a salt thereof.

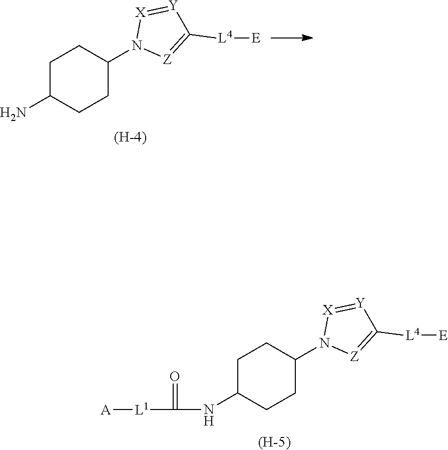

Provided herein are compounds of formula (H-5), or pharmaceutically acceptable salts thereof, synthesized according to the general method described in the schemes above.

In some embodiments, the methods can be performed with enantiopure materials. Compounds disclosed herein, such as compounds of formula (H-5-a), or pharmaceutically acceptable salts thereof, can be synthesized according to the general method described in the schemes below.

Provided herein are methods of synthesizing a compound of formula (H-5-a)

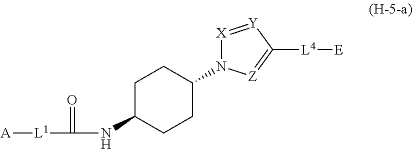

wherein X, Y, and Z, are, independently from each other, selected from the group consisting of NH, NR^{D}, CH, and CR^{D}, provided that:
   at least one of X, Y, and Z, is CH or CR^{D}.
   when Z is NH or NR^{D}, then X is CH or CR^{D}; n
   when X is NH or NR^{D}, and Y is NH or NR^{D}, then Z is CH or CR^{D}.
R^{D}, independently at each occurrence, is selected from the group consisting of C₁-C₆ alkyl, C₁-C₆ haloalkyl, —C(O)OH, —C(O)O(C₁-C₆ alkyl), —C(O)O(C₁-C₆ haloalkyl), and halogen;
A is selected from the group consisting of:
   C₆-C₁₄ aryl optionally substituted with 1, 2, 3, 4, 5, 6, 7, 8, or 9 R^{A} substituents;
   5-14 membered heteroaryl optionally substituted with 1, 2, 3, 4, 5, 6, 7, 8, or 9 R^{A} substituents; and
   8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to the remainder of the molecule;
   wherein R^{L1} is H, C₁-C₆ alkyl, or C₁-C₆ haloalkyl;
   wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L⁴ is a bond or #^{D}—CH₂—O—$^{E}, wherein $^{E} represents the attachment point to E and #^{D} represents the remainder of the molecule;
E is selected from the group consisting of:
   C₃-C₁₄ cycloalkyl optionally substituted with 1, 2, 3, 4, 5, 6, 7, 8, or 9 R^{E} substituents;
   3-14 heterocycloalkyl optionally substituted with 1, 2, 3, 4, 5, 6, 7, 8, or 9 R^{E} substituents;
   C₆-C₁₄ aryl optionally substituted with 1, 2, 3, 4, 5, 6, 7, 8, or 9 R^{E} substituents;
   5-14 membered heteroaryl optionally substituted with 1, 2, 3, 4, 5, 6, 7, 8, or 9 R^{E} substituents; and
   8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, oxo. NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl).

In some embodiments, the method comprises reacting a compound of formula (H-1-a), or a salt thereof, wherein PG is a protecting group suitable to protect an amine group (such as, for example, a tert-butoxycarbonyl group), under suitable conditions to convert the hydroxyl group of the compound of formula (H-1-a) into a leaving group (such as, for example, a methanesulfonate group, a trifluoromethanesulfonate group, or a 4-methylbenzenesulfonate group), thereby giving a compound of formula (H-2-a), or a salt thereof, wherein PG is as defined for the compound of formula (H-1-a). In some embodiments, the method further comprises reacting the compound of formula (H-2-a), or a salt thereof, under conditions suitable to give a compound of formula (H-3-a), or a salt thereof, wherein X, Y, Z, and L⁴, are as defined for the compound of formula (H-5-a), and wherein PG is as defined for the compound of formula (H-1-a).

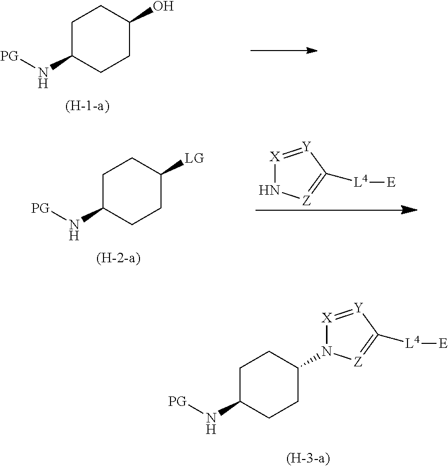

In some embodiments, the method comprises deprotecting a compound of formula (H-3-a), wherein X, Y, Z, and L⁴, are as defined for the compound of formula (H-5-a), and wherein PG is a protecting group suitable to protect an amine group (such as, for example, a tert-butoxycarbonyl group), to give a compound of formula (H-4-a), or a salt thereof, wherein X, Y, Z, and L⁴, are as defined for the compound of formula (H-5-a).

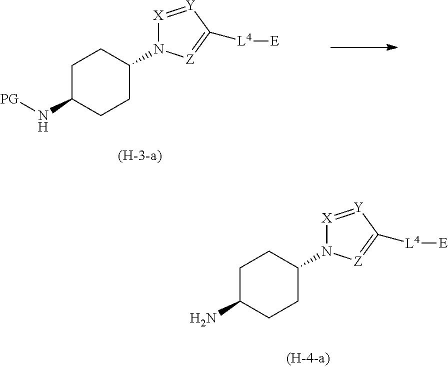

In some embodiments, the method comprises reacting a compound of formula (H-4-a), or a salt thereof, wherein X, Y, Z, and L⁴, are as defined for the compound of formula (H-5-a), to give the compound of formula (H-5-a), or a salt thereof.

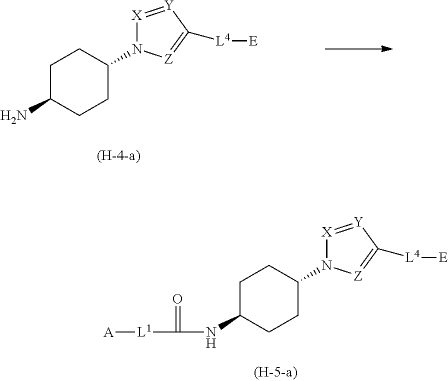

Provided herein are compounds of formula (H-5-a), or pharmaceutically acceptable salts thereof, synthesized according to the general method described in the schemes above.

In some embodiments of the compounds of formula (E-3), (E-4), (E-5), (E-3-a), (E-4-a), (E-5-a), (F-3), (F-4), (F-5), (F-3-a), (F-4-a), (F-5-a), (G-3), (G-4), (G-5), (G-3-a), (G-4-a), (G-5-a), (H-3), (H-4), (H-5), (H-3-a), (H-4-a), and (H-5-a), or the salts thereof, X, Y, and Z, are selected from the group consisting of:

X is CH or CR^{D}, Y is CH or CR^{D}, and Z is CH or CR^{D};

X is NH or NR^{D}, Y is CH or CR^{D}, and Z is CH or CR^{D};

X is CH or CR^{D}, Y is NH or NR^{D}, and Z is CH or CR^{D};

X is CH or CR^{D}, Y is CH or CR^{D}, and Z is NH or NR^{D};

X is NH or NR^{D}, Y is NH or NR^{D}, and Z is CH or CR^{D}; and

X is CH or CR^{D}, Y is NH or NR^{D}, and Z is NH or NR^{D}.

In some embodiments, X, Y, and Z, are selected from the group consisting of:

X is CH, Y is CH, and Z is CH;

X is NH, Y is CH, and Z is CH;

X is CH, Y is NH, and Z is CH;

X is CH, Y is CH, and Z is NH;

X is NH, Y is NH, and Z is CH; and

X is CH, Y is NH, and Z is NH.

In some embodiments of the compounds of formula (E-2), (E-2-a), (F-2), (F-2-a), (G-2), (G-2-a), (H-2), and (H-2-a), LG is methanesulfonate. In some embodiments of the compounds of formula (E-2), (E-2-a), (F-2), (F-2-a), (G-2), (G-2-a), (H-2), and (H-2-a), LG is trifluoromethanesulfonate. In some embodiments of the compounds of formula (E-2), (E-2-a), (F-2), (F-2-a), (G-2), (G-2-a), (H-2), and (H-2-a), or the salts thereof, LG is 4-methylbenzenesulfonate.

In some embodiments of the compounds of formula (A-2), (A-2-a), (A-3), (A-3-a), (A-4), (A-4-a), (A-5), (A-5-a), (A-6), (A-6-a), (A-7), (A-7-a), (B-2), (B-2-a), (B-3), (B-3-a), (B-4), (B-4-a), (B-5), (B-5-a), (B-6), (B-6-a), (B-7), (B-7-a), (C-2), (C-2-a), (C-3), (C-3-a), (C-4), (C-4-a), (C-5), (C-5-a), (C-6), (C-6-a), (C-7), (C-7-a), (D-2), (D-2-a), (D-3), (D-3-a), (D-4), (D-4-a), (D-5), (D-5-a), (D-6), (D-6-a), (D-7), (D-7-a), (E-1), (E-1-a), (E-2), (E-2-a), (E-3), (E-3-a), (F-1), (F-1-a), (F-2), (F-2-a), (F-3), (F-3-a), (G-1), (G-1-a), (G-2), (G-2-a), (G-3), (G-3-a), (H-1), (H-1-a), (H-2), (H-2-a), (H-3), and (H-3-a), or the salts thereof, PG is tert-butoxycarbonyl.

In some embodiments of the compounds of formula (A-10), (A-10-a), (A-11), (A-11-a), (B-10), (B-10-a), (B-11), (B-11-a), (C-10), (C-10-a), (C-11), (C-11-a), (D-10), (D-10-a), (D-11), (D-11-a), (E-5), (E-5-a), (F-5), (F-5-a), (G-5), (G-5-a), (H-5), and (H-5-a), or the salts thereof, A is selected from the group consisting of:

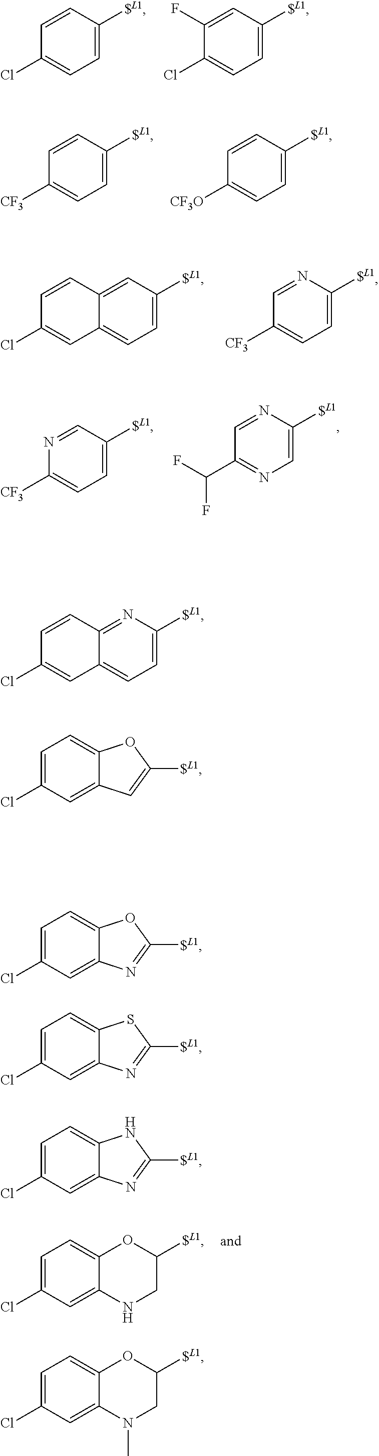

wherein $^{L1} represents the attachment point to L¹. In some embodiments, A is selected from the group consisting of:

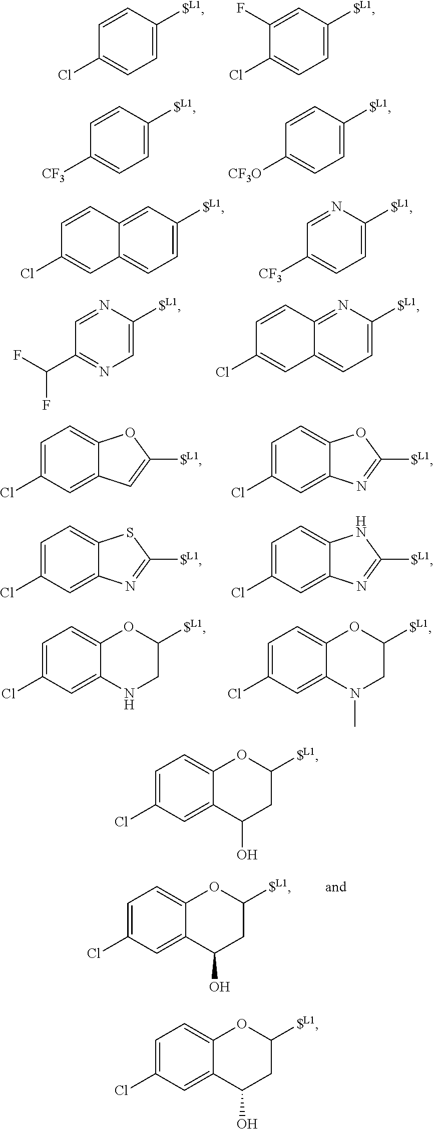

wherein $^{L1} represents the attachment point to L¹. In some embodiments, A is

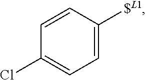

wherein $^{L1} represents the attachment point to L¹. In some embodiments, A is

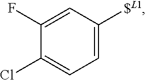

wherein $^{L1} represents the attachment point to L¹. In some embodiments, A is

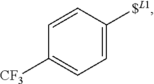

wherein $^{L1} represents the attachment point to L¹. In some embodiments, A is

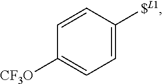

wherein $^{L1} represents the attachment point to L¹. In some embodiments, A is

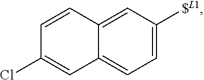

wherein $^{L1} represents the attachment point to L¹. In some embodiments, A is

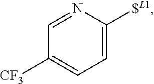

wherein $^{L1} represents the attachment point to L¹. In some embodiments, A is

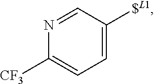

wherein $^{L1} represents the attachment point to L¹. In some embodiments, A is

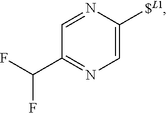

wherein $^{L1} represents the attachment point to L¹. In some embodiments, A is

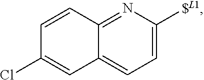

wherein $^{L1} represents the attachment point to L¹. In some embodiments, A is

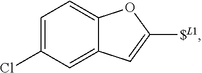

wherein $^{L1} represents the attachment point to L¹. In some embodiments, A is

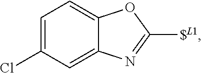

wherein $^{L1} represents the attachment point to L¹. In some embodiments, A is

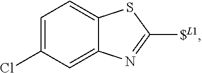

wherein $^{L1} represents the attachment point to L¹. In some embodiments, A is

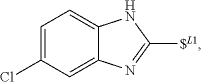

wherein $^{L1} represents the attachment point to L¹. In some embodiments, A is

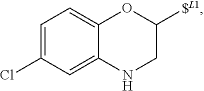

wherein $^{L1} represents the attachment point to L¹. In some embodiments, A is

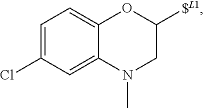

wherein $^{L1} represents the attachment point to L¹. In some embodiments, A is

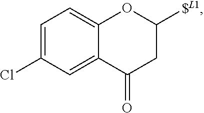

wherein $^{L1} represents the attachment point to L¹. In some embodiments, A is

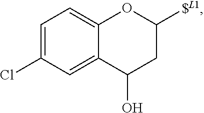

wherein $^{L1} represents the attachment point to L¹. In some embodiments, A is

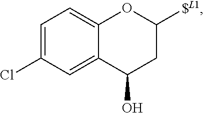

wherein $^{L1} represents the attachment point to L¹. In some embodiments, A is

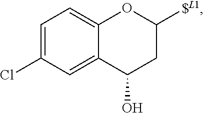

wherein $^{L1} represents the attachment point to L¹.

In some embodiments of the compounds of formula (A-10), (A-10-a), (A-11), (A-11-a), (B-10), (B-10-a), (B-11), (B-11-a), (C-10), (C-10-a), (C-11), (C-11-a), (D-10), (D-10-a), (D-11), (D-11-a), (E-5), (E-5-a), (F-5), (F-5-a), (G-5), (G-5-a), (H-5), and (H-5-a), or the salts thereof, A is C₆-C₁₄ aryl optionally substituted with 1, 2, 3, 4, 5, 6, 7, 8, or 9 R^{A} substituents. In some embodiments, A is C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents. In some embodiments, R^{A} independently at each occurrence, is selected from the group consisting of halogen, C₁-C₆ haloalkyl, and O(C₁-C₆ haloalkyl). In some embodiments, R^{A}, independently at each occurrence, is selected from the group consisting of chloro, fluoro, trifluoromethyl, and trifluoromethoxy. In some embodiments, A is selected from the group consisting of:

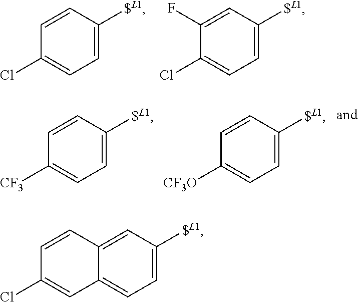

wherein $^{L1} represents the attachment point to L¹.

In some embodiments of the compounds of formula (A-10), (A-10-a), (A-11), (A-11-a), (B-10), (B-10-a), (B-11), (B-l-a), (C-10), (C-10-a), (C-11), (C-l-a), (D-10), (D-10-a), (D-11), (D-11-a), (E-5), (E-5-a), (F-5), (F-5-a), (G-5), (G-5-a), (H-5), and (H-5-a), or the salts thereof, A is 5-14 membered heteroaryl optionally substituted with 1, 2, 3, 4, 5, 6, 7, 8, or 9 R^{A} substituents. In some embodiments, A is 5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents. In some embodiments, R^{A}, independently at each occurrence, is selected from the group consisting of halogen and C₁-C₆ haloalkyl. In some embodiments, R^{A}, independently at each occurrence, is selected from the group consisting of chloro, difluoromethyl, and trifluoromethyl. In some embodiments, A is selected from the group consisting of:

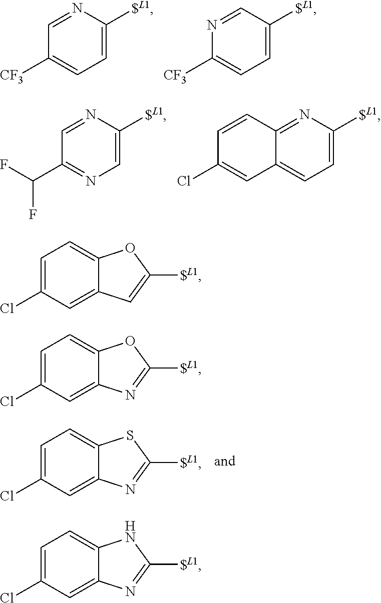

wherein $^{L1} represents the attachment point to L¹.

In some embodiments of the compounds of formula (A-10), (A-10-a), (A-11), (A-11-a), (B-10), (B-10-a), (B-11), (B-l-a), (C-10), (C-10-a), (C-11), (C-l-a), (D-10), (D-10-a), (D-11), (D-11-a), (E-5), (E-5-a), (F-5), (F-5-a), (G-5), (G-5-a), (H-5), and (H-5-a), or the salts thereof, A is 8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, 8-14 membered partially unsaturated fused bicyclic ring moiety, substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, R^{A}, independently at each occurrence, is selected from the group consisting of halogen and C₁-C₆ alkyl. In some embodiments, R^{A}, independently at each occurrence, is selected from the group consisting of chloro and methyl. In some embodiments, A is selected from the group consisting of.

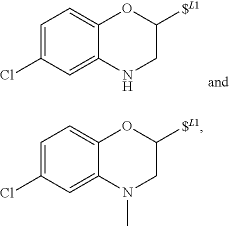

wherein $^{L1} represents the attachment point to L¹.

In some embodiments of the compounds of formula (A-10), (A-10-a), (A-11), (A-11-a), (B-10), (B-10-a), (B-11), (B-l-a), (C-10), (C-10-a), (C-11), (C-l-a), (D-10), (D-10-a), (D-11), (D-11-a), (E-5), (E-5-a), (F-5), (F-5-a), (G-5), (G-5-a), (H-5), and (H-5-a), or the salts thereof, L¹ is selected from the group consisting of a bond, #^{A}—CH₂—$^{L2}, #^{A}—C(CH₃)₂—$^{L2}, #^{A}—CH₂—CH₂—$^{L2}, #^{A}—O—$^{L2}, #^{A}—O—CH₂—$^{L2}, and #^{A}—N(R^{L1})—$^{L2}, wherein #^{A} represents the attachment point to A and $^{L2} represents the attachment point to the remainder of the molecule. In some embodiments, L¹ is a bond. In some embodiments, L¹ is #^{A}—CH₂—$^{L2}, wherein #^{A} represents the attachment point to A and $^{L2} represents the attachment point to the remainder of the molecule. In some embodiments, L¹ is #^{A}—C(CH₃)₂—$^{L2}, wherein #^{A} represents the attachment point to A and $^{L2} represents the attachment point to the remainder of the molecule. In some embodiments, L¹ is #^{A}—CH₂—CH₂—$^{L2}, wherein #^{A} represents the attachment point to A and $^{L2} represents the attachment point to the remainder of the molecule. In some embodiments, L¹ is #^{A}—O—$^{L2}, wherein #^{A} represents the attachment point to A and $^{L2} represents the attachment point to the remainder of the molecule. In some embodiments, L¹ is #^{A}—O—CH₂—$^{L2}, wherein #^{A} represents the attachment point to A and $^{L2} represents the attachment point to the remainder of the molecule. In some embodiments, L¹ is #^{A}—N(R^{L1})—$^{L2}, wherein #^{A} represents the attachment point to A and $^{L2} represents the attachment point to the remainder of the molecule. In some embodiments, L¹ is #^{A}—NH—$^{L2}, wherein #^{A} represents the attachment point to A and $^{L2} represents the attachment point to the remainder of the molecule.

In some embodiments of the compounds of formula (A-10), (A-10-a), (A-11), (A-11-a), (B-10), (B-10-a), (B-11), (B-11-a), (C-10), (C-10-a), (C-11), (C-11-a), (D-10), (D-10-a), (D-11), (D-11-a), (E-5), (E-5-a), (F-5), (F-5-a), (G-5), (G-5-a), (H-5), and (H-5-a), or the salts thereof, or the salts thereof.

### A is

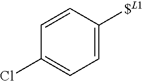

and L¹ is #^{A}—O—CH₂—$^{L2};

### A is

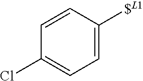

and L¹ is #^{A}—O—CH₂—$^{L2};

### A is

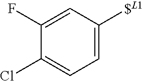

and L¹ is #^{A}—O—CH₂—$^{L2};

### A is

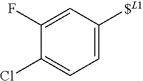

and L¹ is #^{A}—O—CH₂—$^{L2};

### A is

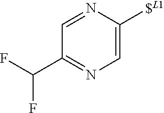

and L¹ is a bond;

### A is

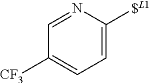

and L¹ is a bond;

### A is

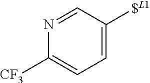

and L¹ is a bond;

### A is

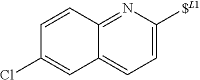

and L¹ is a bond;

### A is

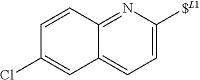

and L¹ is a bond;

### A is

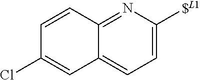

and L¹ is a bond;

### A is

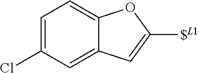

and L¹ is a bond;

### A is

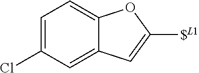

and L¹ is a bond;

### A is

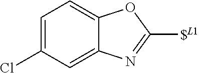

and L¹ is a bond;

### A is

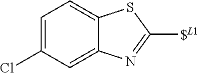

and L¹ is a bond; or

### A is

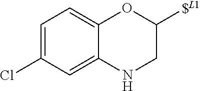

and L¹ is a bond;
wherein $^{L1} represents the attachment point to L¹ and $^{L2} represents the attachment point to the remainder of the molecule. In some embodiments, A is

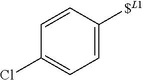

and L¹ is #^{A}—O—CH₂—$^{L2}, wherein $^{L1} represents the attachment point to L¹ and $^{L2} represents the attachment point to the remainder of the molecule. In some embodiments, A is

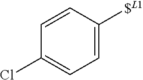

and L¹ is #^{A}—O—CH₂—$^{L2}, wherein $^{L1} represents the attachment point to L¹ and $^{L2} represents the attachment point to the remainder of the molecule. In some embodiments, A is

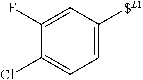

and L¹ is #^{A}—O—CH₂—$^{L2}, wherein $^{L1} represents the attachment point to L¹ and $^{L2} represents the attachment point to the remainder of the molecule. In some embodiments, A is

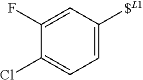

and L¹ is #^{A}—O—CH₂—$^{L2}, wherein $^{L1} represents the attachment point to L¹ and $^{L2} represents the attachment point to the remainder of the molecule. In some embodiments, A is

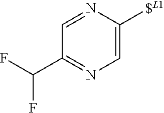

and L¹ is a bond, wherein $^{L1} represents the attachment point to L¹. In some embodiments, A is

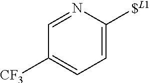

and L¹ is a bond, wherein $^{L1} represents the attachment point to L¹. In some embodiments, A is

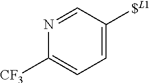

and L¹ is a bond, wherein $^{L1} represents the attachment point to L¹. In some embodiments, A is

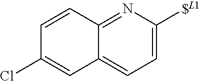

and L¹ is a bond, wherein $^{L1} represents the attachment point to L¹. In some embodiments, A is

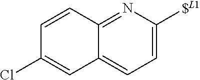

and L¹ is a bond, wherein $^{L1} represents the attachment point to L¹. In some embodiments, A is

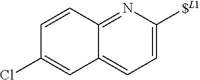

and L¹ is a bond, wherein $^{L1} represents the attachment point to L¹. In some embodiments, A is

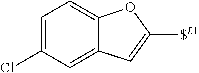

and L¹ is a bond, wherein $^{L1} represents the attachment point to L¹. In some embodiments, A is

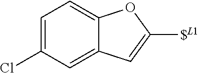

and L¹ is a bond, wherein $^{L1} represents the attachment point to L¹. In some embodiments, A is

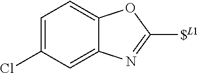

and L¹ is a bond, wherein $^{L1} represents the attachment point to L¹. In some embodiments, A is

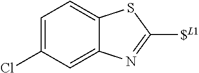

and L¹ is a bond, wherein $^{L1} represents the attachment point to L¹. In some embodiments, A is

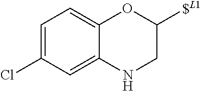

and L¹ is a bond, wherein $^{L1} represents the attachment point to L¹.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, L⁴ is a bond.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, L⁴ is #^{D}—CH₂—O—$^{E}, wherein $^{E} represents the attachment point to E and #^{D} represents the remainder of the molecule.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, E is C₃-C₁₄ cycloalkyl optionally substituted with 1, 2, 3, 4, 5, 6, 7, 8, or 9 R^{E} substituents. In some embodiments, R^{E}, independently at each occurrence, is selected from the group consisting of halogen, C₁-C₆ haloalkyl, OH, O(C₁-C₆ alkyl), and O(C₁-C₆ haloalkyl). In some embodiments, R^{E}, independently at each occurrence, is selected from the group consisting of fluoro, trifluoromethyl, OH, methoxy, and trifluoromethoxy. In some embodiments, E is C₃-C₁₄ cycloalkyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, R^{E}, independently at each occurrence, is selected from the group consisting of fluoro, trifluoromethyl, OH, methoxy, and trifluoromethoxy. In some embodiments, E is cyclohexyl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, R^{E}, independently at each occurrence, is selected from the group consisting of fluoro, trifluoromethyl, OH, methoxy, and trifluoromethoxy. In some embodiments, E is selected from the group consisting of:

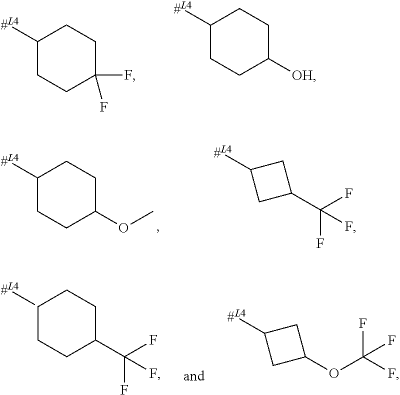

wherein #^{L4} represents the attachment point to L⁴. In some embodiments, E is

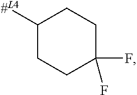

wherein #^{L4} represents the attachment point to L⁴. In some embodiments, E is

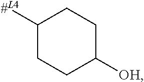

wherein #^{L4} represents the attachment point to L⁴. In some embodiments, E is

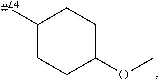

wherein #^{L4} represents the attachment point to L⁴. In some embodiments, E is

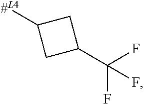

wherein #^{L4} represents the attachment point to L⁴. In some embodiments, E is

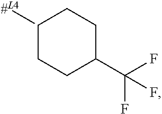

wherein #^{L4} represents the attachment point to L⁴. In some embodiments, E is

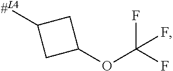

wherein #^{L4} represents the attachment point to L⁴.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, E is 3-14 heterocycloalkyl optionally substituted with 1, 2, 3, 4, 5, 6, 7, 8, or 9 R^{E} substituents. In some embodiments, R^{E}, independently at each occurrence, is selected from the group consisting of halogen, C₁-C₆ alkyl, C₁-C₆ haloalkyl, OH, O(C₁-C₆ alkyl), and O(C₁-C₆ haloalkyl). In some embodiments, R^{E}, independently at each occurrence, is selected from the group consisting of fluoro, methyl, trifluoromethyl, OH, methoxy, and trifluoromethoxy. In some embodiments, E is selected from the group consisting of:

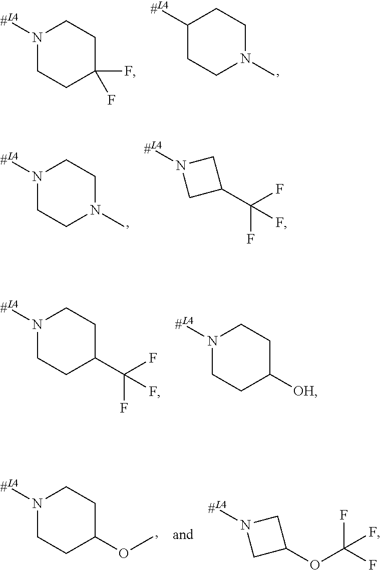

wherein #^{L4} represents the attachment point to L⁴. In some embodiments, E is

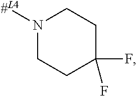

wherein #^{L4} represents the attachment point to L⁴. In some embodiments, E is

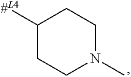

wherein #^{L4} represents the attachment point to L⁴. In some embodiments, E is

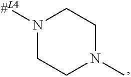

wherein #^{L4} represents the attachment point to L⁴. In some embodiments, E is

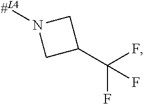

wherein #^{L4} represents the attachment point to L⁴. In some embodiments, E is

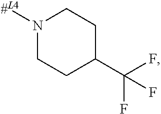

wherein #^{L4} represents the attachment point to L⁴. In some embodiments, E is

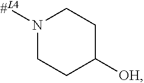

wherein #^{L4} represents the attachment point to L⁴. In some embodiments, E is

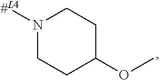

wherein #^{L4} represents the attachment point to L⁴. In some embodiments, E is

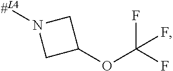

wherein #^{L4} represents the attachment point to L⁴.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, E is C₆-C₁₄ aryl optionally substituted with 1, 2, 3, 4, 5, 6, 7, 8, or 9 R^{E} substituents. In some embodiments, C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, R^{E}, independently at each occurrence, is halogen. In some embodiments, R^{E}, independently at each occurrence, is selected from the group consisting of fluoro and chloro. In some embodiments, E is selected from the group consisting of:

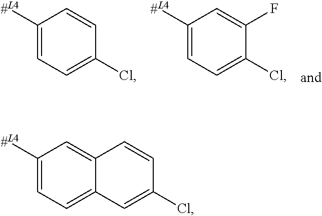

wherein #^{L4} represents the attachment point to L⁴. In some embodiments, E is

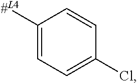

wherein #^{L4} represents the attachment point to L⁴. In some embodiments, E is

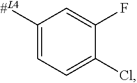

wherein #^{L4} represents the attachment point to L⁴. In some embodiments, E is

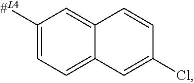

wherein #^{L4} represents the attachment point to L⁴.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, E is 5-14 membered heteroaryl optionally substituted with 1, 2, 3, 4, 5, 6, 7, 8, or 9 R^{E} substituents. In some embodiments, E is 5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents. In some embodiments, R^{E}, independently at each occurrence, is selected from the group consisting of halogen and O(C₁-C₆ alkyl). In some embodiments, R^{E}, independently at each occurrence, is selected from the group consisting of chloro and methoxy. In some embodiments, E is selected from the group consisting of:

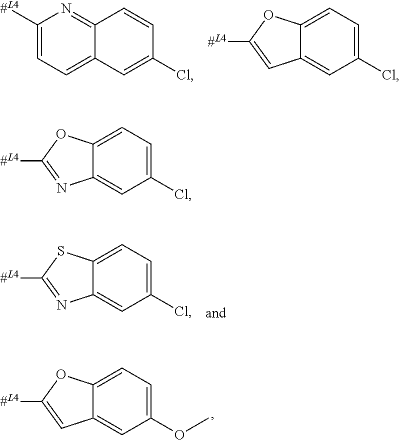

wherein #^{L4} represents the attachment point to L⁴. In some embodiments, E is

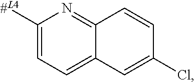

wherein #^{L4} represents the attachment point to L⁴. In some embodiments, E is

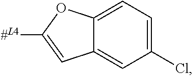

wherein #^{L4} represents the attachment point to L⁴. In some embodiments, E is

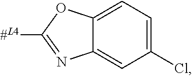

wherein #^{L4} represents the attachment point to L⁴. In some embodiments, E is

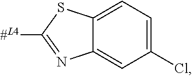

wherein #^{L4} represents the attachment point to L⁴. In some embodiments, E is

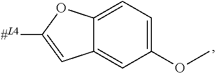

wherein #^{L4} represents the attachment point to L⁴.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, E is 8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, E is 8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl. In some embodiments, R^{E}, independently at each occurrence, is halogen. In some embodiments, R^{E}, independently at each occurrence, is chloro. In some embodiments, E is

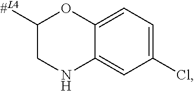

wherein #^{L4} represents the attachment point to L⁴. In some embodiments, R^{E}, independently at each occurrence, is chloro. In some embodiments, E is

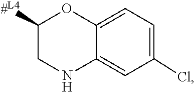

wherein #^{L4} represents the attachment point to L⁴. In some embodiments, R^{E}, independently at each occurrence, is chloro. In some embodiments, E is

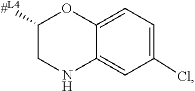

wherein #^{L4} represents the attachment point to L⁴.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof:

### E is

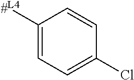

and L⁴ is a bond;

### E is

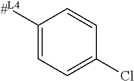

and L⁴ is #^{D}—CH₂—O—$^{E};

### E is

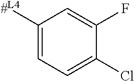

and L⁴ is a bond;

### E is

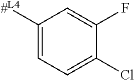

and L⁴ is #^{D}—CH₂—O—$^{E};

### E is

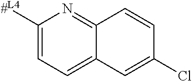

and L⁴ is a bond;

### E is

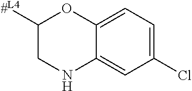

and L⁴ is a bond;

### E is

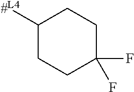

and L⁴ is a bond; or

### E is

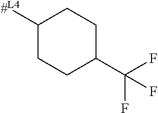

and L⁴ is a bond;
wherein #^{L4} represents the attachment point to L⁴, $^{E} represents the attachment point to E, and #^{D} represents the remainder of the molecule.
In some embodiments E is

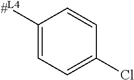

and L⁴ is a bond;

### E is

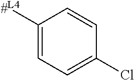

and L⁴ is #^{D}—CH₂—O—$^{E}.

### E is

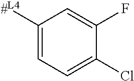

and L4 is a bond;

### E is

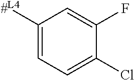

and L⁴ is #^{D}—CH₂—O—$^{E};

### E is

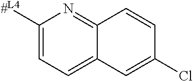

and L⁴ is a bond; or

### E is

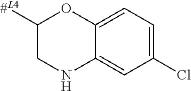

and L⁴ is a bond;
wherein #^{L4} represents the attachment point to L⁴, $^{E} represents the attachment point to E, and #^{D} represents the remainder of the molecule.
In some embodiments, E is

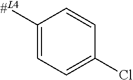

and L⁴ is a bond, wherein #^{L4} represents the attachment point to L⁴ and $^{E} represents the attachment point to E. In some embodiments, E is E is

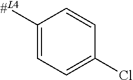

and L⁴ is #^{D}—CH₂—O—$^{E} wherein #^{L4} represents the attachment point to L⁴, $^{E} represents the attachment point to E, and #^{D} represents the remainder of the molecule. In some embodiments, E is

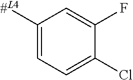

and L⁴ is a bond, wherein #^{L4} represents the attachment point to L⁴ and $^{E} represents the attachment point to E. In some embodiments, E is

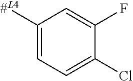

and L⁴ is #^{D}—CH₂—O—$^{E}, wherein #^{L4} represents the attachment point to L⁴, $^{E} represents the attachment point to E, and #^{D} represents the remainder of the molecule. In some embodiments, E is

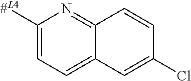

and L⁴ is a bond, wherein #^{L4} represents the attachment point to L⁴ and $^{E} represents the attachment point to E. In some embodiments, E is

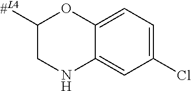

and L⁴ is a bond, wherein #^{L4} represents the attachment point to L⁴ and $^{E} represents the attachment point to E. In some embodiments, E is

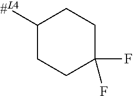

and L⁴ is a bond, wherein #^{L4} represents the attachment point to L⁴ and $^{E} represents the attachment point to E. In some embodiments, E is

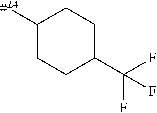

and L⁴ is a bond, wherein #^{L4} represents the attachment point to L⁴ and $^{E} represents the attachment point to E.

In some embodiments of the compounds of formula (A-5), (A-5-a), (A-6), (A-6-a), (A-7), (A-7-a), (A-8), (A-8-a), (A-9), (A-9-a), (A-10), (A-10-a), (A-11), (A-11-a), (B-5), (B-5-a), (B-6), (B-6-a), (B-7), (B-7-a), (B-8), (B-8-a), (B-9), (B-9-a), (B-10), (B-10-a), (B-11), (B-11-a), (C-5), (C-5-a), (C-6), (C-6-a), (C-7), (C-7-a), (C-8), (C-8-a), (C-9), (C-9-a), (C-10), (C-10-a), (C-11), (C-11-a), (D-5), (D-5-a), (D-6), (D-6-a), (D-7), (D-7-a), (D-8), (D-8-a), (D-9), (D-9-a), (D-10), (D-10-a), (D-11), (D-11-a), (E-3), (E-3-a), (E-4), (E-4-a), (E-5), (E-5-a), (F-3), (F-3-a), (F-4), (F-4-a), (F-5), (F-5-a), (G-3), (G-3-a), (G-4), (G-4-a), (G-5), (G-5-a), (H-3), (H-3-a), (H-4), (H-4-a), (H-5), and (H-5-a), or the salts thereof, L⁴ and E are taken together to form a group selected from the group consisting of C₁-C₆ haloalkyl, (C₁-C₆ alkylene)—O—(C₁-C₆ haloalkyl), —O—(C₁-C₆ alkylene)—O—(C₁-C₆ haloalkyl), and —NH—(C₁-C₆ alkylene)—O—(C₁-C₆ haloalkyl). In some embodiments, L⁴ and E are taken together to form a group selected from the group consisting of

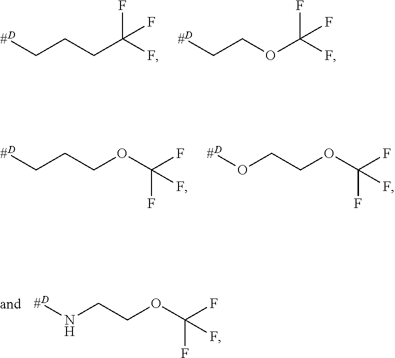

wherein #^{D} represents the attachment point to D. In some embodiments, L⁴ and E are taken together to form

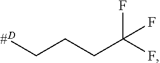

wherein #^{D} represents the attachment point to D. In some embodiments, L⁴ and E are taken together to form

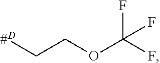

wherein #^{D} represents the attachment point to D. In some embodiments, L⁴ and E are taken together to form

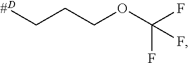

wherein #^{D} represents the attachment point to D. In some embodiments, L⁴ and E are taken together to form

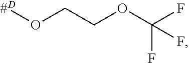

wherein #^{D} represents the attachment point to D. In some embodiments, L⁴ and E are taken together to form

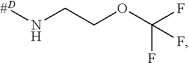

wherein #^{D} represents the attachment point to D.

### EXAMPLES

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

In some cases, stereoisomers are separated to give single enantiomers or diastereomers as single, unknown stereoisomers, and are arbitrarily drawn as single isomers. Where appropriate, information is given on separation method and elution time and order. In the biological examples, compounds tested were prepared in accordance to the synthetic procedures described therein. For any given compound of unknown absolute stereochemistry for which a stereochemistry has been arbitrarily assigned and for which a specific rotation and/or chiral HPLC elution time has been measured, biological data reported for that compound was obtained using the enantiomer or diastereoisomer associated with said specific rotation and/or chiral HPLC elution time.

In some cases, optical rotation was determined on Jasco DIP-360 digital polarimeter at a wavelength of 589 nm (sodium D line) and are reported as [α]_{D}^{T} for a given temperature T (expressed in ° C.). Where appropriate, information is given on solvent and concentration (expressed as g/100 mL).

Abbreviations:
br. s. Broad singlet
DCM Dichloromethane
DIPEA Diisopropylethylamine
DMF N,N-Dimethylformamide
DMSO-d₆ Deuterated dimethylsulfoxide
d Doublet
EtOAc Ethyl acetate
g Gram
HATU (O-(7-azabenzotriazol-1-yl)-N,N,N′,N′-tetramethyluronium hexafluorophosphate)
HPLC High Performance Liquid Chromatography
L Litre
LCMS Liquid Chromatography Mass Spectrometry
MeCN Acetonitrile
MeOH Methanol
mg Milligram
mL Millilitre
mmol Millimoles
m multiplet
NMR Nuclear Magnetic Resonance
q quartet
RT Room temperature
s singlet
TFA trifluoroacetic acid
THF Tetrahydrofuran
TLC Thin layer chromatography
t triplet

### EXAMPLES

### Example 1

### Synthesis of trans-6-chloro-N-((4-(5-(4-chlorophenyl)-1,3,4-oxadiazol-2-yl)cyclohexyl)methyl)quinoline-2-carboxamide

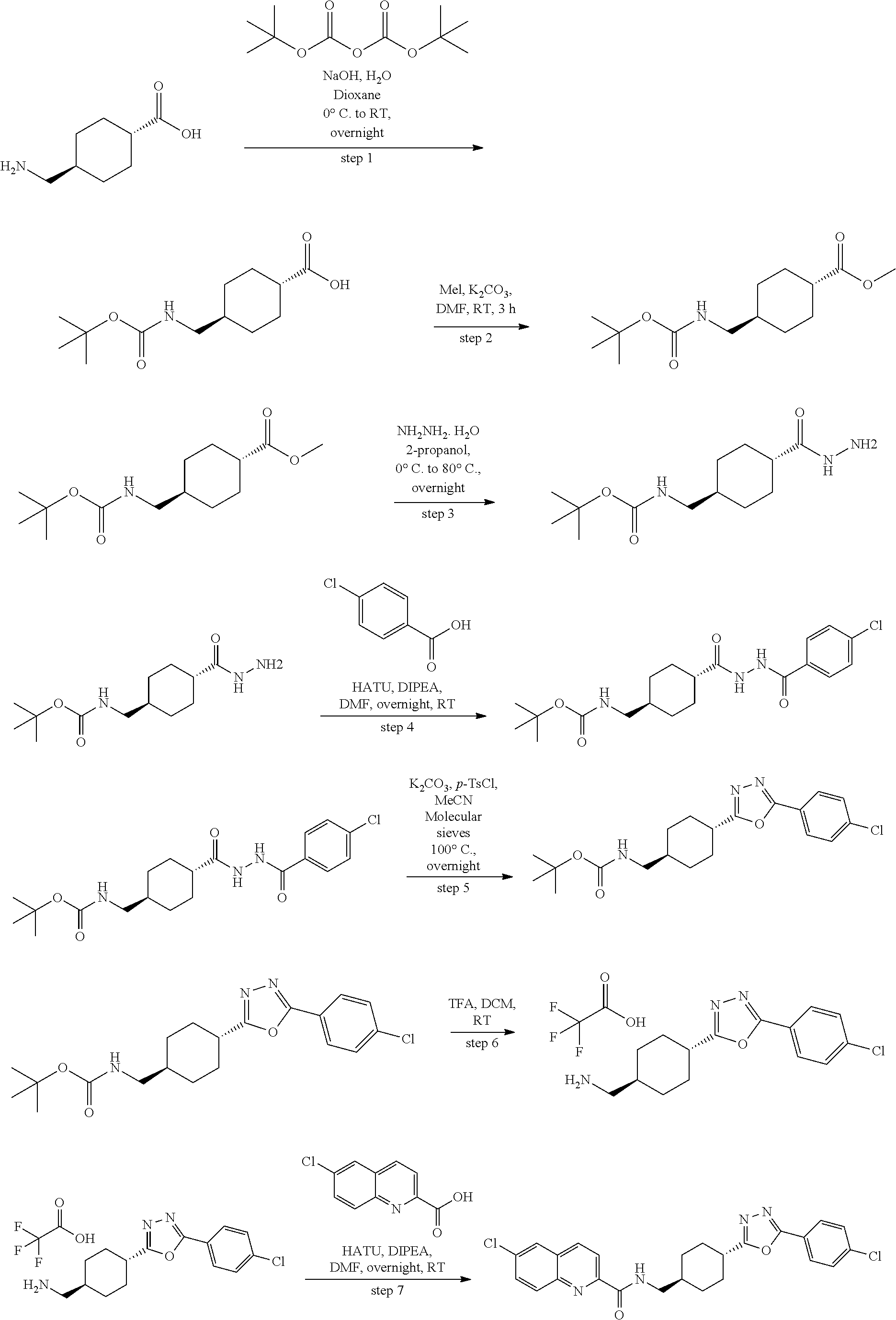

### Step 1: Synthesis of trans-4-(((tert-butoxycarbonyl)amino)methyl)cyclohexane-1-carboxylic acid

To a stirred solution of trans-4-(aminomethyl)cyclohexane-1-carboxylic acid (500 mg, 3.18 mmol, 1.0 eq.), in dioxane (3 mL) was added NaOH (254 mg, 4.8 mmol, 1.5 eq.) and followed by addition of di-tert-butyl dicarbonate (1040 mg, 6.36 mmol, 2.0 eq.) at 0° C. The resulting reaction mixture was allowed to stir at RT overnight. Product formation was confirmed by ¹H NMR. Upon completion, the reaction mixture was concentrated under reduced pressure and the resulting residue was dissolved in water (10 mL), and acidified with 6 M HCl (pH˜3). The resulting solid was filtered off and dried under vacuum to obtain trans-4-(((tert-butoxycarbonyl)amino)methyl)cyclohexane-1-carboxylic acid (800 mg, 97.9% yield) as a white solid. LCMS 258.0 [M+H]⁺; ¹H NMR (400 MHz, DMSO-d₆) δ 11.97 (br. s., 1H), 6.79 (br. s., 1H), 2.75 (t, J=6.1 Hz, 2H), 2.09 (t, J=12.3 Hz, 1H), 1.87 (d, J=11.8 Hz, 2H), 1.69 (d, J=10.5 Hz, 3H), 1.37 (s, 9H), 1.28-1.13 (m, 2H), 0.91-0.68 (m, 2H).

### Step 2: Synthesis of trans-methyl 4-(((tert-butoxycarbonyl)amino)methyl)cyclohexane-1-carboxylate

To a stirred solution of trans-methyl 4-(((tert-butoxycarbonyl)amino)methyl)cyclohexane-1-carboxylate (800 mg, 3.11 mmol, 1 eq.) in DMF (5 mL) was added K₂CO₃ (1288 mg, 9.336 mmol, 2 eq.) and methyl iodide (0.6 mL, 9.336 mmol, 1 eq.) The resulting reaction mixture was stirred at RT for 3 hours under nitrogen atmosphere. Product formation was confirmed by TLC and LCMS. Upon completion, the reaction mixture was poured into ice cold water (10 ml). The resulting solid was filtered off and dried under vacuum to obtain trans-methyl 4-(((tert-butoxycarbonyl)amino)methyl)cyclohexane-1-carboxylate (800 mg, 94.89% yield) as a brown solid. ¹H NMR (400 MHz, DMSO-d₆) δ 6.80 (t, J=5.7 Hz, 1H), 3.57 (s, 3H), 2.81-2.68 (m, 2H), 2.28-2.12 (m, 1H), 2.01-1.77 (m, 2H), 1.69 (d, J=10.5 Hz, 3H), 1.42-1.32 (m, 9H), 1.32-1.10 (m, 2H), 1.00-0.81 (m, 2H).

### Step 3: Synthesis of trans-tert-butyl ((4-(hydrazinecarbonyl)cyclohexyl)methyl)carbamate

To a stirred solution of trans-methyl 4-(((tert-butoxycarbonyl)amino)methyl)cyclohexane-1-carboxylate (1500 mg, 5.523 mmol, 1 eq.) in 2-propanol (6 mL) was added hydrazine hydrate (60%) (1.38 mL, 16.61 mmol, 3.0 eq.). The resulting reaction mixture was stirred at 80° C. overnight. Product formation was confirmed by ¹H NMR. Upon completion, the reaction mixture was cooled to 0° C. The resulting white crystals were filtered off, washed with hexane, and dried under vacuum to obtain trans-tert-butyl ((4-(hydrazinecarbonyl)cyclohexyl)methyl)carbamate (1400 mg, 93.58% yield) as a white solid. LCMS 272.3 [M+H]⁺; ¹H NMR (400 MHz, DMSO-d₆) δ 8.87 (s, 1H), 6.79 (t, J=5.5 Hz, 1H), 4.29 (br. s., 1H), 4.12 (br. s., 1H), 2.75 (t, J=6.1 Hz, 2H), 2.02-1.89 (m, 1H), 1.67 (t, J=11.6 Hz, 3H), 1.37 (s, 9H), 1.35-1.24 (m, 2H), 0.94-0.74 (m, 2H).

### Step 4: Synthesis of trans-tert-butyl ((4-(2-(4-chlorobenzoyl)hydrazine-1-carbonyl)cyclohexyl)methyl)carbamate

To a stirred solution of trans-tert-butyl ((4-(hydrazinecarbonyl)cyclohexyl)methyl)carbamate (1000 mg, 3.69 mmol, 1 eq.) in DMF (5 mL) was added 4-chlorobenzoic acid (691 mg, 4.428 mmol, 1 eq.) and HATU (2103 mg, 5.54 mmol, 1 eq.), followed by the addition of DIPEA (2.0 mL, 11.07 mmol, 3.0 eq.). The resulting reaction mixture was stirred at RT overnight. Product formation was confirmed by LCMS. Upon completion, the reaction mixture was poured into ice cold water (100 ml). The resulting off white solid was filtered and dried under vacuum to obtain trans-tert-butyl ((4-(2-(4-chlorobenzoyl)hydrazine-1-carbonyl)cyclohexyl)methyl)carbamate (450 mg, 29.82% yield) as an off white solid. LCMS 410.1 [M+H]⁺; ¹H NMR (400 MHz, DMSO-d₆) δ 10.37 (br. s., 1H), 9.81 (s, 1H), 7.95-7.78 (m, 2H), 7.65-7.49 (m, 2H), 6.81 (br. s., 1H), 2.87-2.71 (m, 2H), 2.23-2.09 (m, 1H), 1.83-1.66 (m, 3H), 1.48-1.33 (m, 9H), 1.32 (br. s., 2H), 0.98-0.80 (m, 2H).

### Step 5: Synthesis of trans-tert-butyl ((4-(5-(4-chlorophenyl)-1,3,4-oxadiazol-2-yl)cyclohexyl)methyl)carbamate

To a stirred mixture of trans-tert-butyl ((4-(2-(4-chlorobenzoyl)hydrazine-1-carbonyl)cyclohexyl)methyl)carbamate (450 mg, 1.100 mmol, 1 eq.), K₂CO₃ (759 mg, 5.501 mmol, 1 eq.), and molecular sieves (450 mg) in acetonitrile (40 mL) was added 4-toluenesulfonyl chloride (522 mg, 2.751 mmol, 1 eq.) and the resulting reaction mixture was stirred at 100° C. overnight. Product formation was confirmed by LCMS and TLC. Upon completion, the reaction mixture filtered through Celite® and the filtrate was diluted with EtOAc (50 mL). The organic layer was washed with water (25 mL) and brine (25 mL), dried over anhydrous Na₂SO₄, and concentrated. The crude product was crystallized in hexane to obtain trans-tert-butyl ((4-(5-(4-chlorophenyl)-1,3,4-oxadiazol-2-yl)cyclohexyl)methyl)carbamate (300 mg, 69.76% yield) as an off white solid. LCMS 392.3 [M+H]⁺; ¹H NMR (400 MHz, DMSO-d₆) δ 8.06-7.92 (m, 2H), 7.73-7.58 (m, 2H), 6.86 (t, J=5.5 Hz, 1H), 3.00-2.88 (m, 1H), 2.82 (t, J=6.4 Hz, 2H), 2.14 (d, J=11.4 Hz, 2H), 1.80 (d, J=11.0 Hz, 2H), 1.57-1.45 (m, 2H), 1.38 (s, 9H), 1.29-1.17 (m, 1H), 1.09-0.94 (m, 2H).

### Step 6: Synthesis of trans-(4-(5-(4-chlorophenyl)-1,3,4-oxadiazol-2-yl)cyclohexyl)methanamine 2,2,2-trifluoroacetate

To a stirred solution of trans-tert-butyl ((4-(5-(4-chlorophenyl)-1,3,4-oxadiazol-2-yl)cyclohexyl)methyl)carbamate (110 mg, 0.281 mmol, 1 eq.) in DCM (10 mL) was added trifluoroacetic acid (0.1 mL) and the resulting reaction mixture was stirred at RT overnight under nitrogen atmosphere. Product formation was confirmed by LCMS. Upon completion, the reaction mixture was concentrated under reduced pressure. The crude product was crystallized in diethyl ether and dried under vacuum to obtain trans-(4-(5-(4-chlorophenyl)-1,3,4-oxadiazol-2-yl)cyclohexyl)methanamine 2,2,2-trifluoroacetate (100 mg, 87.7% yield) as an off white solid. LCMS 292.2 [M+H]⁺; ¹H NMR (400 MHz, DMSO-d₆) δ 8.00 (d, J=8.3 Hz, 2H), 7.80-7.55 (m, 4H), 2.98 (t, J=12.3 Hz, 1H), 2.81-2.62 (m, 2H), 2.20 (d, J=11.4 Hz, 3H), 1.89 (d, J=12.3 Hz, 2H), 1.64-1.42 (m, 3H), 1.25-1.01 (m, 2H).

### Step 7: Synthesis of trans-6-chloro-N-((4-(5-(4-chlorophenyl)-1,3,4-oxadiazol-2-yl)cyclohexyl)methyl)quinoline-2-carboxamide

To a stirred solution of trans-(4-(5-(4-chlorophenyl)-1,3,4-oxadiazol-2-yl)cyclohexyl)methanamine 2,2,2-trifluoroacetate (50 mg, 0.1234 mmol, 1 eq.), 6-chloroquinoline-2-carboxylic acid (25.5 mg, 0.1234 mmol, 1 eq.) and HATU (71 mg, 0.185 mmol, 1.5 eq.) in DMF (1 mL) was added DIPEA (0.07 mL, 0.379 mmol, 3 eq.), and the resulting reaction mixture was stirred at RT overnight. Product formation was confirmed by LCMS. Upon completion, the reaction mixture was poured into ice cold water (10 ml) and extracted with ethyl acetate (2×20 mL). The combined organic layers were washed with water (4×10 mL) and brine solution (1×10 mL), dried over anhydrous Na₂SO₄, and concentrated under reduced pressure. The crude product was crystallized in hexane/pentane (1:1), filtered, and dried under vacuum to obtain trans-6-chloro-N-((4-(5-(4-chlorophenyl)-1,3,4-oxadiazol-2-yl)cyclohexyl)methyl)quinoline-2-carboxamide (Compound 1-55 mg, 93.2% yield) as a white solid. LCMS 481.4 [M+H]⁺; ¹H NMR (400 MHz, DMSO-d₆) δ 8.99 (br. s., 1H), 8.55 (d, J=8.3 Hz, 1H), 8.25 (d, J=2.2 Hz, 1H), 8.24-8.15 (m, 2H), 8.08-7.93 (m, J=8.8 Hz, 2H), 7.93-7.83 (m, 1H), 7.70-7.52 (m, J=8.3 Hz, 2H), 2.99 (t, J=11.6 Hz, 1H), 2.18 (d, J=12.7 Hz, 3H), 1.89 (d, J=11.0 Hz, 2H), 1.72 (br. s., 1H), 1.61-1.42 (m, 3H), 1.26-1.09 (m, 2H).

### Example 2

### Synthesis of trans-5-chloro-N-((4-(5-(4-chlorophenyl)-1,3,4-oxadiazol-2-yl)cyclohexyl)methyl)benzofuran-2-carboxamide

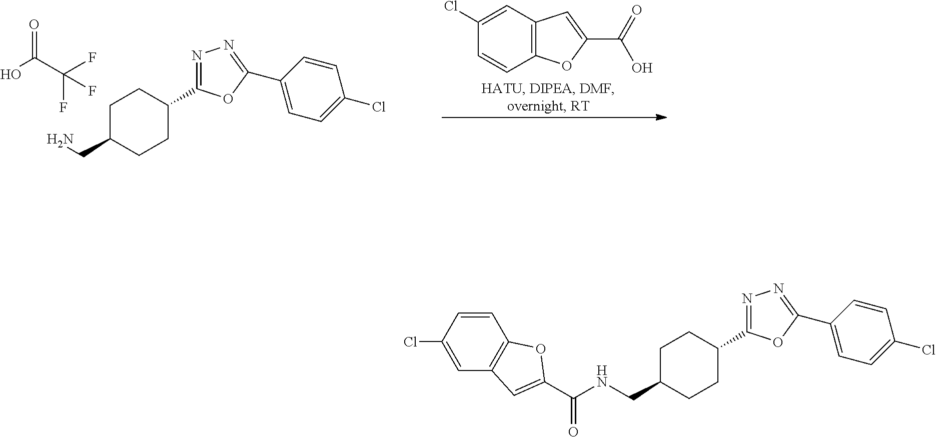

To a stirred solution of trans-(4-(5-(4-chlorophenyl)-1,3,4-oxadiazol-2-yl)cyclohexyl)methanamine 2,2,2-trifluoroacetate (70 mg, 0.173 mmol, 1 eq.), 5-chlorobenzofuran-2-carboxylic acid (34 mg, 0.173 mmol, 1 eq.) and HATU (98 mg, 0.259 mmol, 1.5 eq.) in DMF (3 mL) was added DIPEA (0.1 mL, 0.518 mmol, 3 eq.) and the resulting reaction mixture was stirred at RT overnight. Product formation was confirmed by LCMS. Upon completion, the reaction mixture was poured into ice cold water (50 ml). The resulting solid was filtered off and dried under vacuum to obtain a crude solid which was crystallized in diethyl ether/pentane (1:1) to obtain trans-5-chloro-N-((4-(5-(4-chlorophenyl)-1,3,4-oxadiazol-2-yl)cyclohexyl)methyl)benzofuran-2-carboxamide (Compound 2-50 mg, 61.7% yield) as an off white solid. LCMS 470.4 [M+H]⁺; ¹H NMR (400 MHz, DMSO-d₆) δ 8.91-8.78 (m, 1H), 8.00 (d, J=8.8 Hz, 2H), 7.87 (d, J=2.2 Hz, 1H), 7.76-7.58 (m, 3H), 7.55-7.42 (m, 2H), 3.19 (t, J=6.4 Hz, 2H), 2.98 (t, J=12.1 Hz, 1H), 2.17 (d, J=11.0 Hz, 2H), 1.87 (d, J=9.6 Hz, 2H), 1.65 (br. s., 1H), 1.58-1.42 (m, 2H), 1.23-1.04 (m, 2H).

### Example 3

### Synthesis of trans-2-(4-chloro-3-fluorophenoxy)-N-((4-(5-(4-chlorophenyl)-1,3,4-oxadiazol-2-yl)cyclohexyl)methyl)acetamide

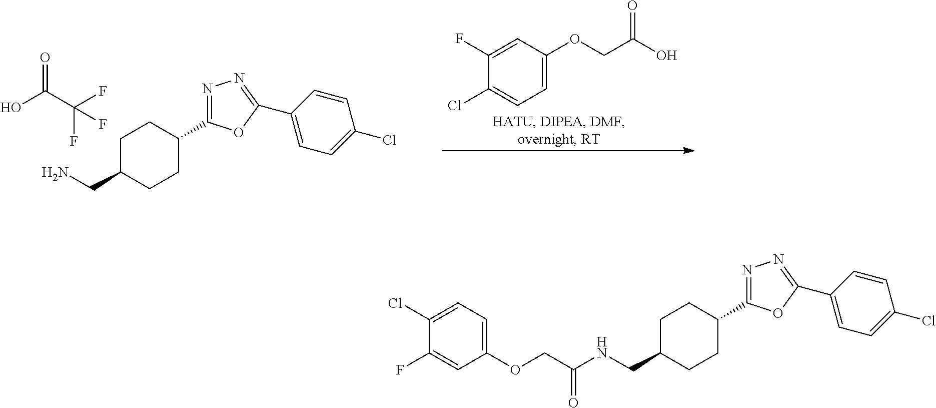

To a stirred solution of trans-(4-(5-(4-chlorophenyl)-1,3,4-oxadiazol-2-yl)cyclohexyl)methanamine 2,2,2-trifluoroacetate (50 mg, 0.1234 mmol, 1 eq.), 2-(4-chloro-3-fluorophenoxy)acetic acid (25 mg, 0.1234 mmol, 1 eq.), and HATU (71 mg, 0.185 mmol, 1.5 eq.) in DMF (1 mL) was added DIPEA (0.07 mL, 0.379 mmol, 3 eq.) and the resulting reaction mixture was stirred at RT overnight. Product formation was confirmed by LCMS. Upon completion, the reaction mixture was poured into ice cold water (50 ml). The resulting solid was filtered off, dried under vacuum and crystallized in diethyl ether/pentane (1:1) to obtain trans-2-(4-chloro-3-fluorophenoxy)-N-((4-(5-(4-chlorophenyl)-1,3,4-oxadiazol-2-yl)cyclohexyl)methyl)acetamide (Compound 3-25 mg, 42.5% yield) as an off white solid. LCMS 478.4 [M+H]⁺; ¹H NMR (400 MHz, DMSO-d₆) δ 8.16 (br. s., 1H), 8.05-7.90 (m, J=8.3 Hz, 2H), 7.73-7.58 (m, J=8.3 Hz, 2H), 7.51 (t, J=8.8 Hz, 1H), 7.08 (dd, J=3.1, 11.4 Hz, 1H), 6.86 (d, J=7.0 Hz, 1H), 4.55 (s, 2H), 3.07-2.95 (m, 2H), 2.95-2.85 (m, 1H), 2.14 (d, J=11.4 Hz, 2H), 1.78 (d, J=10.5 Hz, 2H), 1.57-1.33 (m, 3H), 1.13-0.98 (m, 2H).

### Example 4

### Synthesis of trans-2-(4-chlorophenoxy)-N-((-4-(5-(4-chlorophenyl)-1,3,4-oxadiazol-2-yl)cyclohexyl)methyl)acetamide

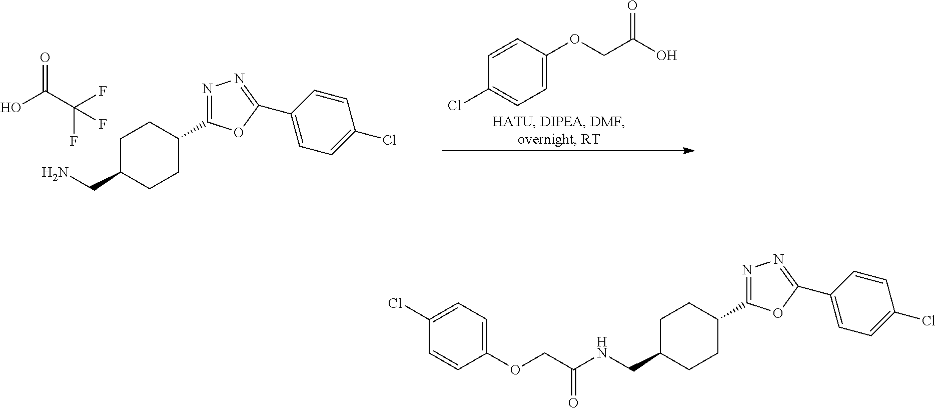

To a stirred solution of trans-(4-(5-(4-chlorophenyl)-1,3,4-oxadiazol-2-yl)cyclohexyl)methanamine 2,2,2-trifluoroacetate (50 mg, 0.1234 mmol, 1 eq.), 2-(4-chlorophenoxy)acetic acid (25 mg, 0.1234 mmol, 1 eq.) and HATU (71 mg, 0.185 mmol, 1.5 eq.) in DMF (1 mL) was added DIPEA (0.07 mL, 0.379 mmol, 3 eq.) and the resulting reaction mixture was stirred at RT overnight. Product formation was confirmed by LCMS. Upon completion, the reaction mixture was poured into ice cold water (50 ml). The resulting solid was filtered off, dried under vacuum, and crystallized in diethyl ether/pentane (1:1) to obtain trans-2-(4-chlorophenoxy)-N-((-4-(5-(4-chlorophenyl)-1,3,4-oxadiazol-2-yl)cyclohexyl)methyl)acetamide (Compound 4-53 mg, 93.6% yield) as an off white solid. LCMS 460.4 [M+H]⁺; ¹H NMR (400 MHz, DMSO-d₆) δ 8.13 (t, J=5.9 Hz, 1H), 8.05-7.92 (m, J=8.8 Hz, 2H), 7.74-7.58 (m, J=8.8 Hz, 2H), 7.42-7.27 (m, J=9.2 Hz, 2H), 7.06-6.91 (m, J=9.2 Hz, 2H), 4.51 (s, 2H), 3.03 (t, J=6.4 Hz, 2H), 2.93 (t, J=12.1 Hz, 1H), 2.13 (d, J=11.4 Hz, 2H), 1.77 (d, J=11.0 Hz, 2H), 1.57-1.38 (m, 3H), 1.13-1.00 (m, 2H).

### Example 5

### Synthesis of trans-6-chloro-N-(4-(5-(4-chlorophenyl)-1,3,4-oxadiazol-2-yl)cyclohexyl)quinoline-2-carboxamide

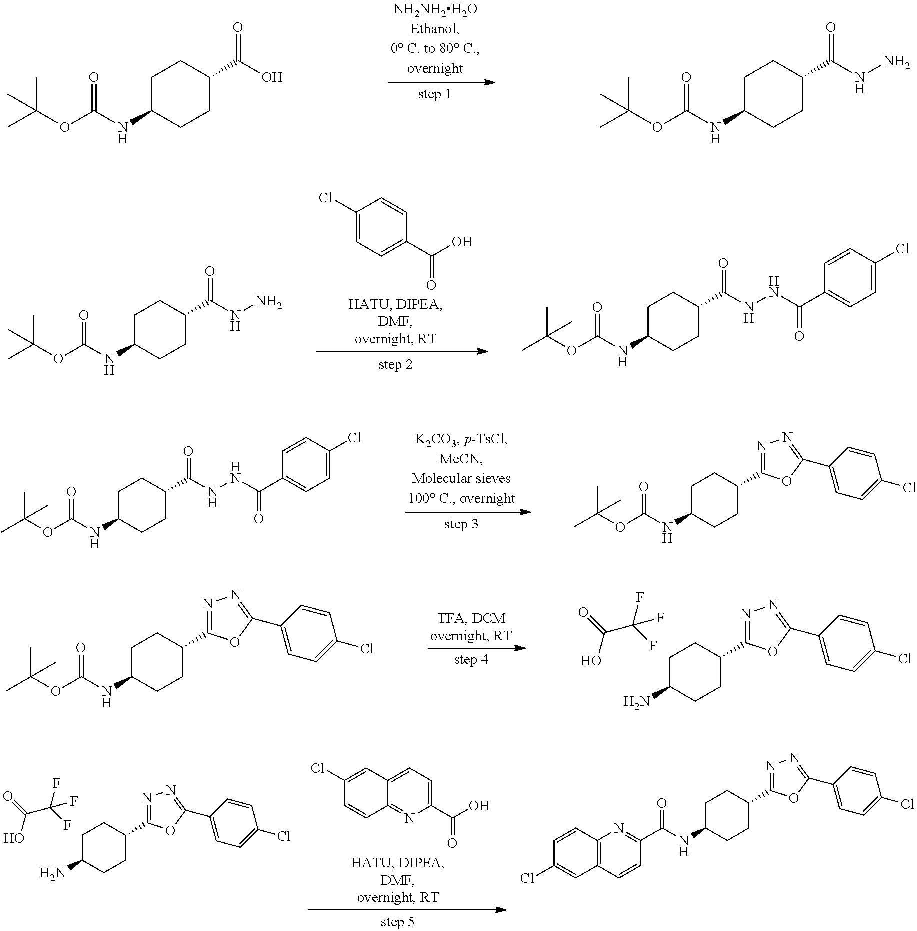

### Step 1: Synthesis of trans-tert-butyl (4-(hydrazinecarbonyl)cyclohexyl)carbamate

To a stirred solution of trans-4-((tert-butoxycarbonyl)amino)cyclohexanecarboxylic acid (500 mg, 1.943 mmol, 1 eq.) in ethanol (10 mL) was added hydrazine hydrate (80%) (0.4 mL, 5.83 mmol, 3.0 eq.), and the resulting reaction mixture was heated at 100° C. overnight. Product formation was confirmed by NMR. Upon completion, the reaction mixture was cooled to 0° C. Resulting white crystals were filtered off and washed with hexane and dried under vacuum to obtain trans-tert-butyl (4-(hydrazinecarbonyl)cyclohexyl)carbamate (250 mg, 50.1% yield) as a white solid. ¹H NMR (400 MHz, DMSO-d₆) δ 8.87 (s, 1H), 6.70 (d, J=7.0 Hz, 1H), 4.12 (br. s., 2H), 3.13 (br. s., 1H), 1.95-1.85 (m, 1H), 1.77 (d, J=10.5 Hz, 2H), 1.64 (d, J=11.4 Hz, 2H), 1.39-1.28 (m, 11H), 1.11 (q, J=12.7 Hz, 2H).

### Step 2: Synthesis of trans-tert-butyl (4-(2-(4-chlorobenzoyl)hydrazine-1-carbonyl)cyclohexyl)carbamate

To a stirred solution of trans-tert-butyl (4-(hydrazinecarbonyl)cyclohexyl)carbamate (250 mg, 0.973 mmol, 1 eq.), 4-chlorobenzoic acid (152 mg, 0.973 mmol, 1 eq.), and HATU (545 mg, 1.46 mmol, 1 eq.) in DMF (5 mL) was added DIPEA (0.7 mL, 3.89 mmol, 4.0 eq.), and the resulting reaction mixture was stirred at RT overnight. Product formation was confirmed by LCMS. Upon completion, the reaction mixture was poured into ice cold water (100 ml). The resulting off white solid was filtered off and dried under vacuum to obtain trans-tert-butyl (4-(2-(4-chlorobenzoyl)hydrazine-1-carbonyl)cyclohexyl)carbamate (300 mg, 78.13% yield) as an off white solid. LCMS 396.3 [M+H]⁺; ¹H NMR (400 MHz, DMSO-d₆) δ 10.38 (s, 1H), 9.82 (s, 1H), 7.96-7.81 (m, J=8.3 Hz, 2H), 7.65-7.46 (m, J=8.8 Hz, 2H), 6.74 (d, J=8.3 Hz, 1H), 3.17 (br. s., 1H), 2.15 (t, J=11.6 Hz, 1H), 1.91-1.62 (m, 4H), 1.51-1.26 (m, 11H), 1.25-1.07 (m, 2H).

### Step 3: Synthesis of trans-tert-butyl (4-(5-(4-chlorophenyl)-1,3,4-oxadiazol-2-yl)cyclohexyl)carbamate

To a stirred mixture of trans-tert-butyl (4-(2-(4-chlorobenzoyl)hydrazine-1-carbonyl)cyclohexyl)carbamate (1000 mg, 2.53 mmol, 1 eq.), K₂CO₃ (1750 mg, 12.65 mmol, 5.0 eq.), and molecular sieves (1000 mg) in acetonitrile (50 mL) was added 4-toluenesulfonyl chloride (1200 mg, 6.329 mmol, 2.5 eq.), and the resulting reaction mixture was heated at 100° C. overnight. Product formation was confirmed by LCMS and TLC. Upon completion, the reaction mixture was filtered through Celite®. The filtrate was dissolved in EtOAc (100 mL). The organic layer was washed with water (50 mL) and brine (50 mL), dried over anhydrous Na₂SO₄, and concentrated. The crude product was washed with pentane to obtain trans-tert-butyl (4-(5-(4-chlorophenyl)-1,3,4-oxadiazol-2-yl)cyclohexyl)carbamate (900 mg, 94.33% yield) as an off white solid. LCMS 378.3 [M+H]⁺; ¹H NMR (400 MHz, DMSO-d₆) δ 8.06-7.87 (m, J=8.8 Hz, 2H), 7.74-7.54 (m, 2H), 6.84 (d, J=7.9 Hz, 1H), 2.94-2.87 (m, 1H), 2.14 (d, J=12.3 Hz, 2H), 1.89 (d, J=10.5 Hz, 2H), 1.64-1.54 (m, 3H), 1.39 (s, 9H), 1.28-1.06 (m, 2H).

### Step 4: Synthesis of trans-4-(5-(4-chlorophenyl)-1,3,4-oxadiazol-2-yl)cyclohexan-1-amine 2,2,2-trifluoroacetate

To a stirred solution of trans-tert-butyl (4-(5-(4-chlorophenyl)-1,3,4-oxadiazol-2-yl)cyclohexyl)carbamate (500 mg, 1.326 mmol, 1 eq.) in DCM (10 mL) was added trifluoroacetic acid (0.4 mL, 3.978 mmol, 3 eq.), and the resulting reaction mixture was stirred at RT overnight under nitrogen atmosphere. Product formation was confirmed by LCMS. Upon completion, the reaction mixture was concentrated under reduced pressure. The crude product was crystallized in diethyl ether, dried under vacuum to obtain trans-4-(5-(4-chlorophenyl)-1,3,4-oxadiazol-2-yl)cyclohexan-1-amine 2,2,2-trifluoroacetate (450 mg, 86.8% yield) as an off white solid. LCMS 278.2 [M+H]⁺; ¹H NMR (400 MHz, DMSO-d₆) δ 8.06-7.93 (m, 2H), 7.86 (br. s., 2H), 7.74-7.61 (m, J=8.8 Hz, 2H), 3.10 (br. s., 1H), 3.06-2.93 (m, 1H), 2.21 (d, J=11.0 Hz, 2H), 2.11-1.99 (m, 2H), 1.72-1.54 (m, 2H), 1.53-1.41 (m, 2H).

### Step 5: Synthesis of trans-6-chloro-N-(4-(5-(4-chlorophenyl)-1,3,4-oxadiazol-2-yl)cyclohexyl)quinoline-2-carboxamide

To a stirred solution of trans-4-(5-(4-chlorophenyl)-1,3,4-oxadiazol-2-yl)cyclohexan-1-amine 2,2,2-trifluoroacetate (100 mg, 0.256 mmol, 1 eq.), 6-chloroquinoline-2-carboxylic acid (53 mg, 0.256 mmol, 1 eq.) and HATU (146 mg, 0.384 mmol, 1.5 eq.) in DMF (4 mL) was added DIPEA (0.2 mL, 1.023 mmol, 4 eq.) and the resulting reaction mixture was stirred at RT overnight. Product formation was confirmed by LCMS. Upon completion, the reaction mixture was poured into ice cold water (100 ml). The resulting solid was filtered off and dried under vacuum to obtain crude product which was crystallized in methanol and washed with diethyl ether to obtain trans-6-chloro-N-(4-(5-(4-chlorophenyl)-1,3,4-oxadiazol-2-yl)cyclohexyl)quinoline-2-carboxamide (Compound 5-85 mg, 71.4% yield) as a white solid. LCMS 467.4 [M+H]⁺; ¹H NMR (400 MHz, DMSO-d₆) δ 8.74 (d, J=8.3 Hz, 1H), 8.55 (d, J=8.3 Hz, 1H), 8.26 (br. s., 1H), 8.24-8.09 (m, 2H), 8.09-7.94 (m, J=8.3 Hz, 2H), 7.89 (d, J=8.8 Hz, 1H), 7.78-7.59 (m, J=8.3 Hz, 2H), 3.95 (br. s., 1H), 3.03 (br. s., 1H), 2.23 (br. s., 2H), 2.04 (br. s., 2H), 1.84-1.58 (m, 4H).

### Example 6

### Synthesis of trans-5-chloro-N-(4-(5-(4-chlorophenyl)-1,3,4-oxadiazol-2-yl)cyclohexyl)benzofuran-2-carboxamide

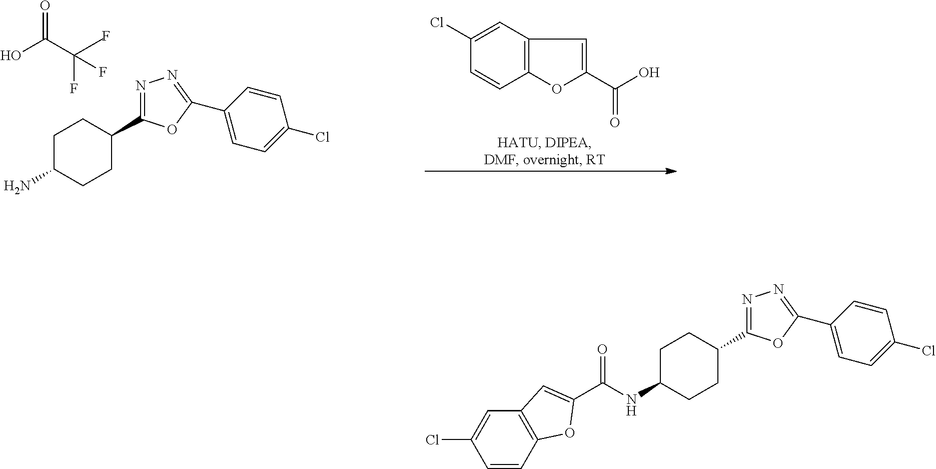

To a stirred solution of trans-4-(5-(4-chlorophenyl)-1,3,4-oxadiazol-2-yl)cyclohexan-1-amine 2,2,2-trifluoroacetate (100 mg, 0.256 mmol, 1 eq.), 5-chlorobenzofuran-2-carboxylic acid (50 mg, 0.256 mmol, 1 eq.), and HATU (146 mg, 0.383 mmol, 1.5 eq.) in DMF (4 mL) was added DIPEA (0.2 mL, 1.022 mmol, 4 eq.) and the resulting reaction mixture was stirred at RT overnight. Product formation was confirmed by LCMS. Upon completion, the reaction mixture was poured into ice cold water (50 ml). The resulting solid was filtered off and dried under vacuum and washed with diethyl ether/pentane (1:1) to obtain trans-5-chloro-N-(4-(5-(4-chlorophenyl)-1,3,4-oxadiazol-2-yl)cyclohexyl)benzofuran-2-carboxamide (Compound 6-108 mg, 93.10% yield) as an off white solid. LCMS 456.4 [M+H]⁺; ¹H NMR (400 MHz, DMSO-d₆) δ 8.70 (d, J=7.9 Hz, 1H), 8.10-7.91 (m, 2H), 7.88 (s, 1H), 7.77-7.59 (m, 3H), 7.55 (s, 1H), 7.54-7.39 (m, 1H), 3.87 (br. s., 1H), 3.06-2.95 (m, 1H), 2.22 (d, J=12.7 Hz, 2H), 1.99 (d, J=11.0 Hz, 2H), 1.79-1.50 (m, 4H).

### Example 7

### Synthesis of trans-2-(4-chloro-3-fluorophenoxy)-N-(4-(5-(4-chlorophenyl)-1,3,4-oxadiazol-2-yl)cyclohexyl)acetamide

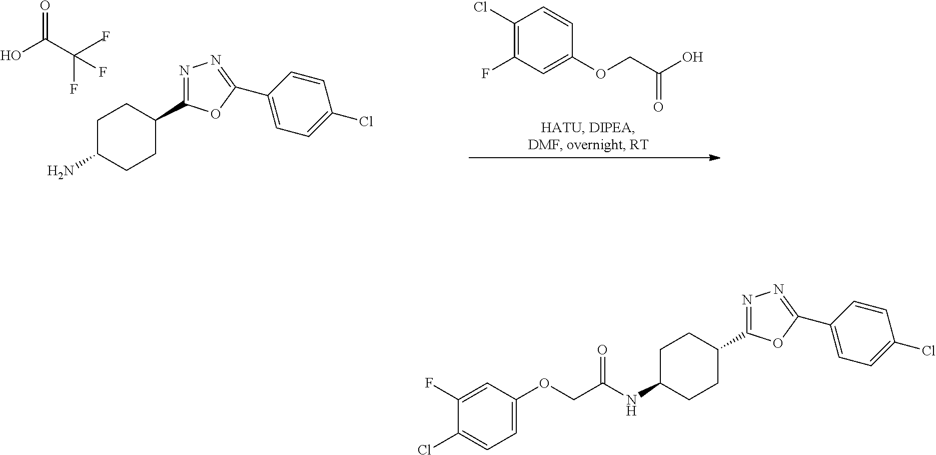

To a stirred solution of trans-4-(5-(4-chlorophenyl)-1,3,4-oxadiazol-2-yl)cyclohexan-1-amine 2,2,2-trifluoroacetate (100 mg, 0.256 mmol, 1 eq.), 2-(4-chloro-3-fluorophenoxy)acetic acid (52 mg, 0.256 mmol, 1 eq.) and HATU (146 mg, 0.383 mmol, 1.5 eq.) in DMF (4 mL) was added DIPEA (0.2 mL, 1.022 mmol, 4 eq.) and the resulting reaction mixture was stirred at RT overnight. Product formation was confirmed by LCMS. Upon completion, the reaction mixture was poured into ice cold water (50 ml). The resulting solid was filtered off and dried under vacuum and crystallized in diethyl ether/pentane (1:1) to obtain trans-2-(4-chloro-3-fluorophenoxy)-N-(4-(5-(4-chlorophenyl)-1,3,4-oxadiazol-2-yl)cyclohexyl)acetamide (Compound 7-110 mg, 93.22% yield) as an off white solid. LCMS 464.4 [M+H]⁺; ¹H NMR (400 MHz, DMSO-d₆) δ 8.06 (d, J=7.9 Hz, 1H), 8.00 (d, J=8.8 Hz, 2H), 7.67 (d, J=8.8 Hz, 2H), 7.50 (t, J=8.8 Hz, 1H), 7.08 (dd, J=2.6, 11.4 Hz, 1H), 6.86 (dd, J=1.8, 8.8 Hz, 1H), 3.70 (d, J=7.9 Hz, 1H), 2.99 (t, J=11.8 Hz, 1H), 2.18 (d, J=11.0 Hz, 2H), 1.90 (d, J=10.1 Hz, 2H), 1.73-1.58 (m, 2H), 1.52-1.35 (m, 2H).

### Example 8

### Synthesis of trans-2-(4-chlorophenoxy)-N-(4-(5-(4-chlorophenyl)-1,3,4-oxadiazol-2-yl)cyclohexyl)acetamide

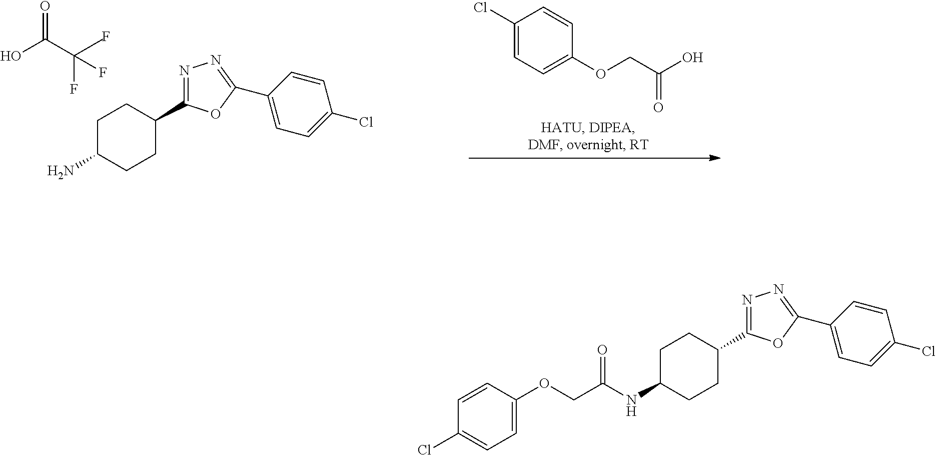

To a stirred solution of trans-4-(5-(4-chlorophenyl)-1,3,4-oxadiazol-2-yl)cyclohexan-1-amine 2,2,2-trifluoroacetate (100 mg, 0.256 mmol, 1 eq.), 2-(4-chloro-3-fluorophenoxy)acetic acid (48 mg, 0.256 mmol, 1 eq.) and HATU (146 mg, 0.383 mmol, 1.5 eq.) in DMF (4 mL) was added DIPEA (0.2 mL, 1.022 mmol, 4 eq.) and the resulting reaction mixture was stirred at RT overnight. Product formation was confirmed by LCMS. Upon completion, the reaction mixture was poured into ice cold water (50 ml). The resulting solid was filtered off and dried under vacuum and crystallized in diethyl ether/pentane (1:1) to obtain trans-2-(4-chlorophenoxy)-N-(4-(5-(4-chlorophenyl)-1,3,4-oxadiazol-2-yl)cyclohexyl)acetamide (Compound 8-112 mg, 98.5% yield) as an off white solid. LCMS 446.4 [M+H]⁺; ¹H NMR (400 MHz, DMSO-d₆) δ 8.10-7.95 (m, 2H), 7.67 (d, J=8.8 Hz, 2H), 7.39-7.23 (m, 2H), 7.05-6.82 (m, 2H), 3.81-3.64 (m, 1H), 3.02-2.89 (m, 1H), 2.17 (d, J=11.4 Hz, 2H), 1.89 (d, J=10.1 Hz, 2H), 1.75-1.57 (m, 2H), 1.48-1.34 (m, 2H).

### Example 9

### Synthesis of trans-6-chloro-N-(4-(5-((4-chlorophenoxy)methyl)-1,3,4-oxadiazol-2-yl)cyclohexyl)quinoline-2-carboxamide

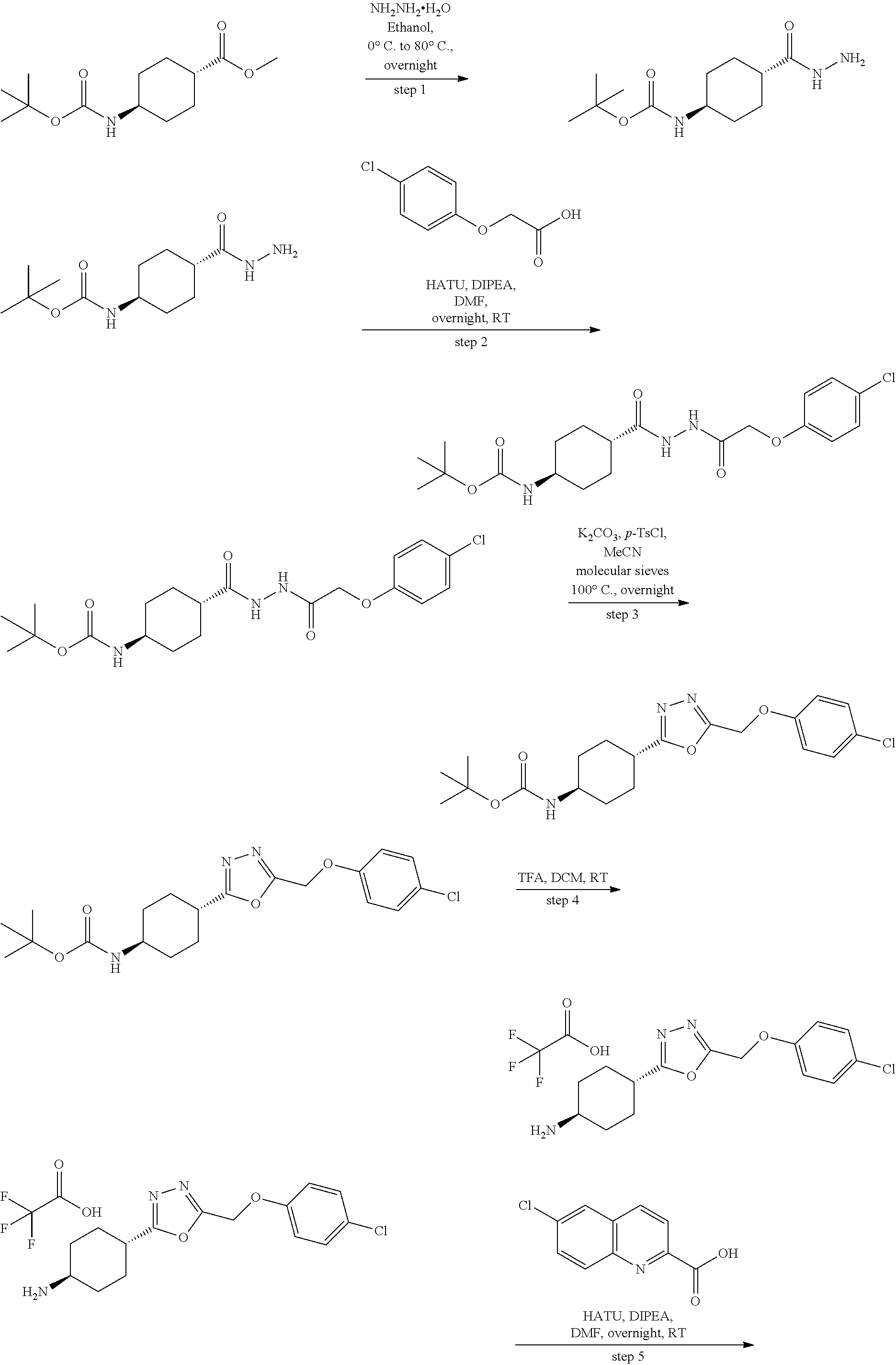

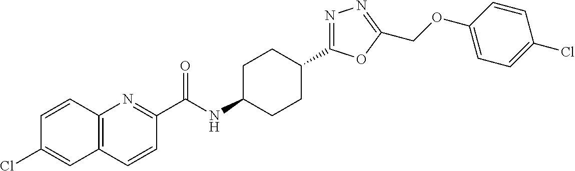 [SMILES: O=C(N[C@H]1CC[C@H](c2nnc(COc3ccc(Cl)cc3)o2)CC1)c1ccc2cc(Cl)ccc2n1]

### Step 1: Synthesis of trans-tert-butyl (4-(hydrazinecarbonyl)cyclohexyl)carbamate

To a stirred solution of trans-methyl 4-((tert-butoxycarbonyl)amino)cyclohexane-1-carboxylate (500 mg, 1.943 mmol, 1 eq.) in ethanol (10 mL) was added hydrazine hydrate (80%) (0.4 mL, 5.83 mmol, 3.0 eq.), and the resulting reaction mixture was heated at 100° C. overnight. Product formation was confirmed by ¹H NMR. Upon completion, the reaction mixture was cooled to 0° C. Resulting crystals were filtered off, washed with hexane, and dried under vacuum to obtain trans-tert-butyl (4-(hydrazinecarbonyl)cyclohexyl)carbamate (250 mg, 50.1% yield) as a white solid. ¹H NMR (400 MHz, DMSO-d₆) δ 8.87 (s, 1H), 6.70 (d, J=7.0 Hz, 1H), 4.12 (br. s., 2H), 3.13 (br. s., 1H), 1.95-1.85 (m, 1H), 1.77 (d, J=10.5 Hz, 2H), 1.64 (d, J=11.4 Hz, 2H), 1.39-1.28 (m, 11H), 1.11 (q, J=12.7 Hz, 2H).

### Step 2: Synthesis of trans-tert-butyl (4-(2-(2-(4-chlorophenoxy)acetyl)hydrazine-1-carbonyl)cyclohexyl)carbamate

To a stirred solution of trans-tert-butyl (4-(hydrazinecarbonyl)cyclohexyl)carbamate (330 mg, 1.28 mmol, 1 eq.), 2-(4-chlorophenoxy)acetic acid (287 mg, 1.54 mmol, 1 eq.) and HATU (730 mg, 1.92 mmol, 1.5 eq.) in DMF (10 mL) was added DIPEA (0.7 mL, 3.89 mmol, 4.0 eq.) and the resulting reaction mixture was stirred at RT overnight. Product formation was confirmed by LCMS. Upon completion, the reaction mixture was poured into ice cold water (100 ml). The resulting off white solid was filtered off and dried under vacuum to obtain trans-tert-butyl (4-(2-(2-(4-chlorophenoxy)acetyl)hydrazine-1-carbonyl)cyclohexyl)carbamate (220 mg, 40.44% yield) as a white solid. LCMS 426.2 [M+H]⁺; ¹H NMR (400 MHz, DMSO-d₆) δ 10.04 (br. s., 1H), 9.74 (br. s., 1H), 7.35 (d, J=9.2 Hz, 2H), 7.10-6.92 (m, 2H), 6.73 (d, J=8.3 Hz, 1H), 4.66-4.47 (m, 2H), 3.15 (br. s., 1H), 2.10 (t, J=11.8 Hz, 1H), 1.90-1.66 (m, 4H), 1.43 (br. s., 2H), 1.37 (s, 9H), 1.23-1.03 (m, 2H).

### Step 3: Synthesis of trans-tert-butyl (4-(5-((4-chlorophenoxy)methyl)-1,3,4-oxadiazol-2-yl)cyclohexyl)carbamate

To a stirred mixture of trans-tert-butyl (4-(2-(2-(4-chlorophenoxy)acetyl)hydrazine-1-carbonyl)cyclohexyl)carbamate (220 mg, 0.5176 mmol, 1 eq.), K₂CO₃ (357 mg, 2.586 mmol, 5.0 eq.), and molecular sieves (220 mg) in acetonitrile (15 mL) was added 4-toluenesulfonyl chloride (246 mg, 1.29 mmol, 2.5 eq.), and the resulting reaction mixture was heated at 100° C. overnight. Product formation was confirmed by LCMS and TLC. Upon completion, the reaction mixture was filtered through Celite®. The filtrate was diluted with EtOAc (100 mL), washed with water (50 mL) and brine (50 mL), dried over anhydrous Na₂SO₄, and concentrated. The crude product was washed with pentane to obtain trans-tert-butyl (4-(5-((4-chlorophenoxy)methyl)-1,3,4-oxadiazol-2-yl)cyclohexyl)carbamate (210 mg, 99.05% yield) as an off white solid. LCMS 408.2 [M+H]⁺; ¹H NMR (400 MHz, DMSO-d₆) δ 7.47 (d, J=7.9 Hz, 1H), 7.37 (d, J=8.8 Hz, 1H), 7.16-7.01 (m, 2H), 6.83 (d, J=7.9 Hz, 1H), 5.37 (s, 2H), 2.87 (t, J=11.6 Hz, 1H), 2.34-2.22 (m, 1H), 2.06 (d, J=12.3 Hz, 2H), 1.86 (d, J=11.0 Hz, 2H), 1.57-1.42 (m, 2H), 1.38 (s, 9H), 1.30-1.20 (m, 2H).

### Step 4: Synthesis of trans-4-(5-((4-chlorophenoxy)methyl)-1,3,4-oxadiazol-2-yl)cyclohexan-1-amine 2,2,2-trifluoroacetate

To a stirred solution of trans-tert-butyl (4-(5-((4-chlorophenoxy)methyl)-1,3,4-oxadiazol-2-yl)cyclohexyl)carbamate (210 mg, 0.516 mmol, 1 eq.) in DCM (10 mL) was added trifluoroacetic acid (0.2 mL, 2.578 mmol, 3.0 eq.), and the resulting reaction mixture was stirred at RT overnight under nitrogen atmosphere. Product formation was confirmed by LCMS. Upon completion, the reaction mixture was concentrated under reduced pressure. The crude product was crystallized in diethyl ether and dried under vacuum to obtain trans-4-(5-((4-chlorophenoxy)methyl)-1,3,4-oxadiazol-2-yl)cyclohexan-1-amine 2,2,2-trifluoroacetate (200 mg, 92.16% yield) as an off white solid. LCMS 308.1 [M+H]⁺; ¹H NMR (400 MHz, DMSO-d₆) δ 7.84 (br. s., 2H), 7.47 (d, J=8.3 Hz, 1H), 7.40-7.33 (m, 1H), 7.14-7.00 (m, 2H), 5.38 (s, 2H), 3.08 (br. s., 1H), 3.00-2.88 (m, 1H), 2.12 (d, J=11.8 Hz, 2H), 2.00 (d, J=10.5 Hz, 2H), 1.63-1.49 (m, 2H), 1.49-1.33 (m, 2H).

### Step 5: Synthesis of trans-6-chloro-N-(4-(5-((4-chlorophenoxy)methyl)-1,3,4-oxadiazol-2-yl)cyclohexyl)quinoline-2-carboxamide

To a stirred solution of trans-4-(5-((4-chlorophenoxy)methyl)-1,3,4-oxadiazol-2-yl)cyclohexan-1-amine 2,2,2-trifluoroacetate (50 mg, 0.119 mmol, 1 eq.), 6-chloroquinoline-2-carboxylic acid (30 mg, 0.143 mmol, 1 eq.), and HATU (68 mg, 0.178 mmol, 1.5 eq.) in DMF (1.5 mL) was added DIPEA (0.1 mL, 0.475 mmol, 4 eq.), and the resulting reaction mixture was stirred at RT overnight. Product formation was confirmed by LCMS. Upon completion, the reaction mixture was poured into ice cold water (100 ml). The resulting solid was filtered off and dried under vacuum to obtain a crude solid which was crystallized in diethyl ether to obtain trans-6-chloro-N-(4-(5-((4-chlorophenoxy)methyl)-1,3,4-oxadiazol-2-yl)cyclohexyl)quinoline-2-carboxamide (Compound 9-25 mg, 42.37%) as a white solid. LCMS 497.4 [M+H]⁺; ¹H NMR (400 MHz, DMSO-d₆) δ 8.70 (d, J=8.3 Hz, 1H), 8.53 (d, J=7.9 Hz, 1H), 8.23 (br. s., 1H), 8.19-8.13 (m, 2H), 7.87 (d, J=9.2 Hz, 1H), 7.46-7.30 (m, J=7.0 Hz, 2H), 7.20-6.98 (m, J=7.5 Hz, 2H), 5.38 (s, 2H), 3.90 (br. s., 1H), 2.97 (br. s., 1H), 2.14 (br. s., 2H), 1.97 (br. s., 2H), 1.64 (br. s., 4H).

### Example 10

### Synthesis of trans-5-chloro-N-(4-(5-((4-chlorophenoxy)methyl)-1,3,4-oxadiazol-2-yl)cyclohexyl)benzofuran-2-carboxamide

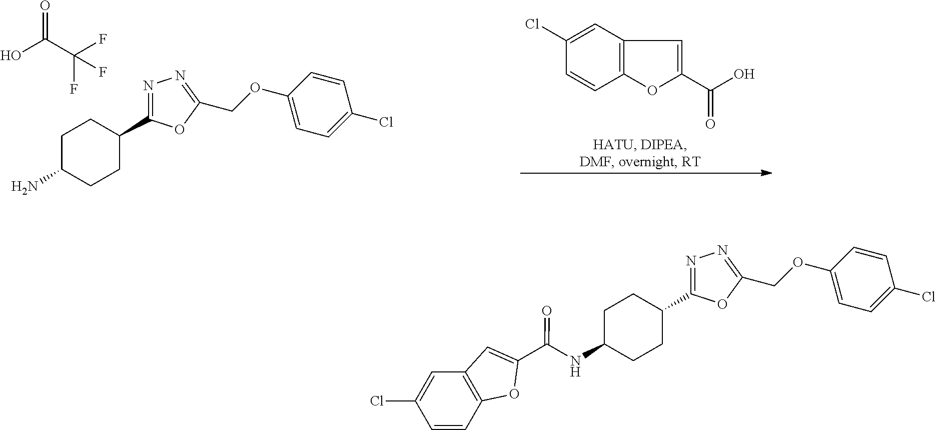

To a stirred solution of trans-4-(5-((4-chlorophenoxy)methyl)-1,3,4-oxadiazol-2-yl)cyclohexan-1-amine 2,2,2-trifluoroacetate (50 mg, 0.119 mmol, 1 eq.), 5-chlorobenzofuran-2-carboxylic acid (28 mg, 0.143 mmol, 1 eq.), and HATU (68 mg, 0.178 mmol, 1.5 eq.) in DMF (1.5 mL) was added DIPEA (0.1 mL, 0.475 mmol, 4 eq.), and the resulting reaction mixture was stirred at RT overnight. Product formation was confirmed by LCMS. Upon completion, the reaction mixture was poured into ice cold water (100 ml). The resulting solid was filtered off and dried under vacuum to obtain a crude solid which was crystallized in diethyl ether to obtain trans-5-chloro-N-(4-(5-((4-chlorophenoxy)methyl)-1,3,4-oxadiazol-2-yl)cyclohexyl)benzofuran-2-carboxamide (Compound 10-12 mg, 20.79% yield) as an off white solid. LCMS 486.4 [M+H]⁺; H NMR (400 MHz, DMSO-d₆) δ 8.66 (d, J=7.5 Hz, 1H), 7.85 (br. s., 1H), 7.75-7.63 (m, 1H), 7.52 (s, 1H), 7.46 (d, J=6.6 Hz, 1H), 7.37 (d, J=8.8 Hz, 2H), 7.08 (d, J=9.2 Hz, 2H), 5.37 (s, 2H), 3.82 (br. s., 1H), 2.91 (br. s., 1H), 2.08 (d, J=18.9 Hz, 2H), 1.92 (br. s., 2H), 1.55 (br. s., 4H).

### Example 11

### Synthesis of trans-6-chloro-N-(4-(5-((4-chlorophenoxy)methyl)-1,3,4-oxadiazol-2-yl)cyclohexyl)-3,4-dihydro-2H-benzo[b][1,4]oxazine-2-carboxamide

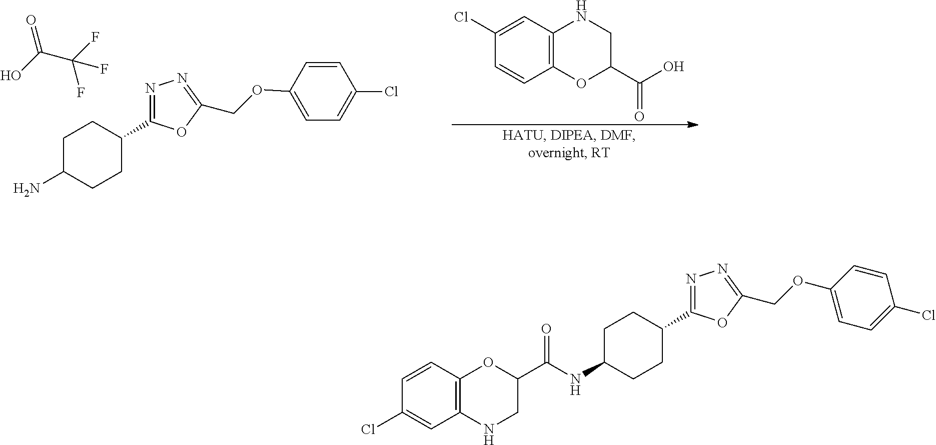

To a stirred solution of trans-4-(5-((4-chlorophenoxy)methyl)-1,3,4-oxadiazol-2-yl)cyclohexan-1-amine 2,2,2-trifluoroacetate (50 mg, 0.119 mmol, 1 eq.), 6-chloro-3,4-dihydro-2H-benzo[b][1,4]oxazine-2-carboxylic acid (30 mg, 0.143 mmol, 1 eq.), and HATU (68 mg, 0.178 mmol, 1.5 eq.) in DMF (1.5 mL) was added DIPEA (0.1 mL, 0.475 mmol, 4 eq.), and the resulting reaction mixture was stirred at RT overnight. Product formation was confirmed by LCMS. Upon completion, the reaction mixture was poured into ice cold water (100 ml). The resulting solid was filtered off and dried under vacuum. The crude product was purified by flash chromatography (3% MeOH in DCM as an eluent) to obtain trans-6-chloro-N-(4-(5-((4-chlorophenoxy)methyl)-1,3,4-oxadiazol-2-yl)cyclohexyl)-3,4-dihydro-2H-benzo[b][1,4]oxazine-2-carboxamide (Compound 11—5 mg, 8.3% yield) as an off white solid. LCMS 503.4 [M+H]⁺; ¹H NMR (400 MHz, DMSO-d₆) δ 7.85 (br. s., 1H), 7.36 (d, J=8.8 Hz, 2H), 7.07 (d, J=9.6 Hz, 2H), 7.00 (br. s., 1H), 6.78 (br. s., 1H), 6.58 (br. s., 1H), 6.49 (br. s., 1H), 6.17 (br. s., 2H), 5.36 (s, 2H), 4.42 (br. s., 1H), 3.63 (br. s., 1H), 2.90 (br. s., 2H), 2.06 (br. s., 2H), 1.81 (br. s., 2H), 1.55 (br. s., 1H), 1.42 (d, J=12.3 Hz, 2H).

### Example 12

### Synthesis of trans-2-(4-chloro-3-fluorophenoxy)-N-(4-(5-((4-chlorophenoxy)methyl)-1,3,4-oxadiazol-2-yl)cyclohexyl)acetamide

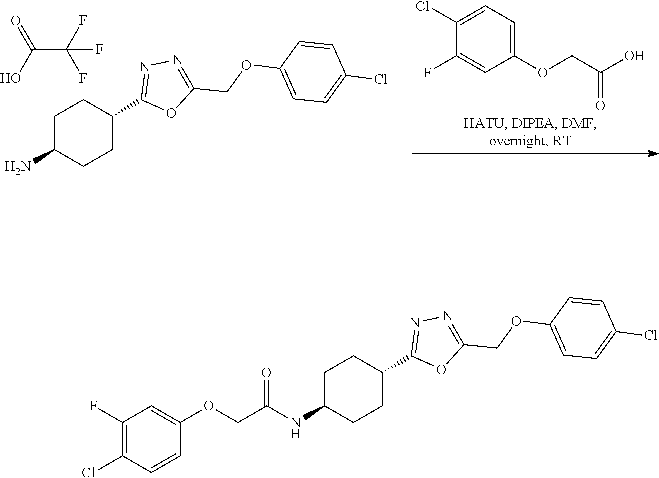

To a stirred solution of trans-4-(5-((4-chlorophenoxy)methyl)-1,3,4-oxadiazol-2-yl)cyclohexan-1-amine 2,2,2-trifluoroacetate (70 mg, 0.1662 mmol, 1 eq.), 2-(4-chloro-3-fluorophenoxy)acetic acid (34 mg, 0.166 mmol, 1 eq.), and HATU (95 mg, 0.249 mmol, 1.5 eq.) in DMF (2 mL) was added DIPEA (0.12 mL, 0.665 mmol, 4 eq.), and the resulting reaction mixture was stirred at RT overnight. Product formation was confirmed by LCMS. Upon completion, the reaction mixture was poured into ice cold water (50 ml). The resulting solid was filtered off and dried under vacuum and the crude product was crystallized in diethyl ether/pentane (1:1) to obtain trans-2-(4-chloro-3-fluorophenoxy)-N-(4-(5-((4-chlorophenoxy)methyl)-1,3,4-oxadiazol-2-yl)cyclohexyl)acetamide (Compound 12-20 mg, 24.42% yield) as an off white solid. LCMS 494.4 [M+H]⁺; ¹H NMR (400 MHz, DMSO-d₆) δ 8.09 (d, J=7.5 Hz, 1H), 7.49 (t, J=8.6 Hz, 1H), 7.37 (d, J=8.3 Hz, 2H), 7.13-6.91 (m, 3H), 6.85 (d, J=8.8 Hz, 1H), 5.37 (br. s., 2H), 4.51 (br. s., 2H), 3.65 (br. s., 1H), 2.93 (br. s., 1H), 2.09 (d, J=12.7 Hz, 2H), 1.86 (d, J=11.0 Hz, 2H), 1.58-1.49 (m, 2H), 1.41 (d, J=11.8 Hz, 2H).

### Example 13

### Synthesis of trans-2-(4-chloro-3-fluorophenoxy)-N-(4-(5-((4-chloro-3-fluorophenoxy)methyl)-1,3,4-oxadiazol-2-yl)cyclohexyl)acetamide

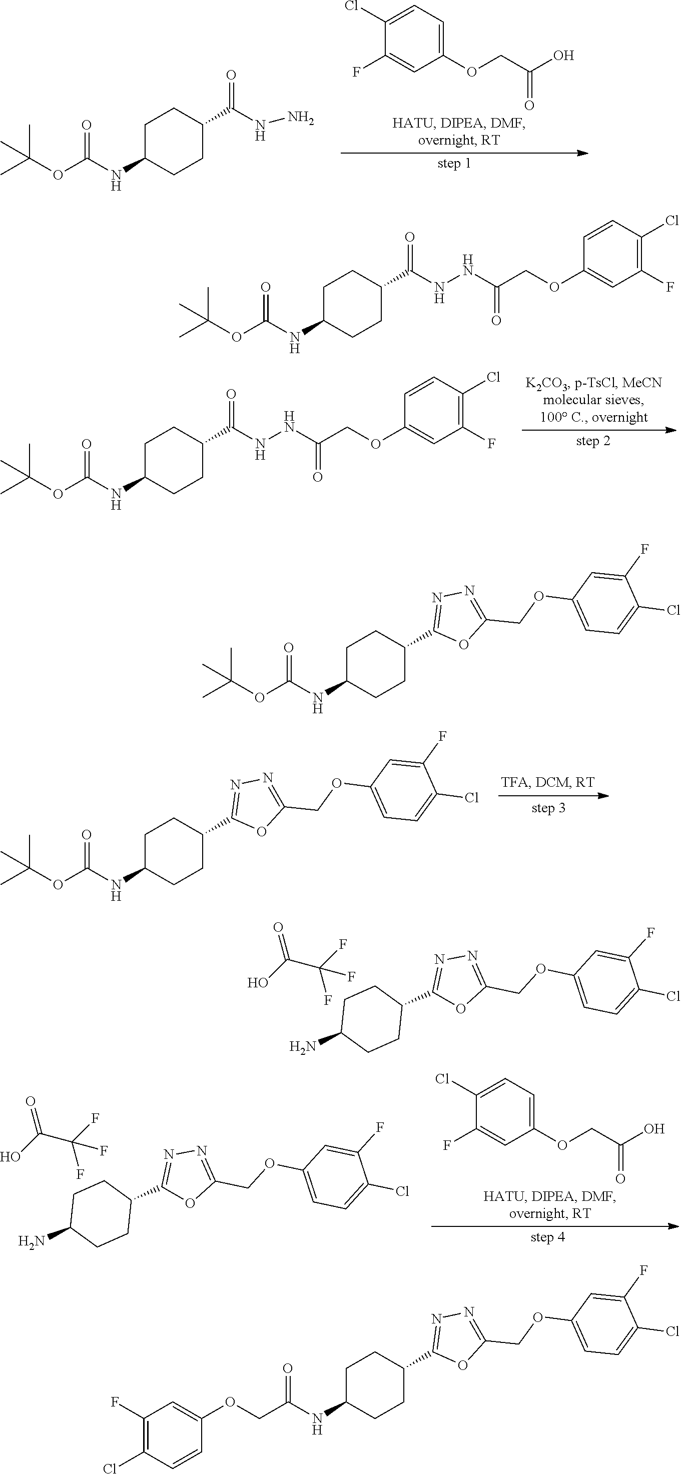

### Step 1: Synthesis of trans-tert-butyl (4-(2-(2-(4-chloro-3-fluorophenoxy)acetyl)hydrazinecarbonyl)cyclohexyl)carbamate

To a stirred mixture of trans-tert-butyl (4-(hydrazinecarbonyl)cyclohexyl)carbamate (400 mg, 1.556 mmol, 1 eq.), 2-(4-chloro-3-fluorophenoxy)acetic acid (381 mg, 1.867 mmol, 1.2 eq.), and HATU (887 mg, 2.334 mmol, 1.5 eq.) in DMF (10 mL) was added DIPEA (1.2 mL, 6.224 mmol, 4.0 eq.), and the resulting reaction mixture was stirred at RT overnight. Product formation was confirmed by LCMS. Upon completion, the reaction mixture was poured into ice cold water (100 ml). The resulting off white solid was filtered off and dried under vacuum to obtain trans-tert-butyl (4-(2-(2-(4-chloro-3-fluorophenoxy)acetyl)hydrazinecarbonyl)cyclohexyl)carbamate (600 mg, 87.08% yield) as an off white solid. LCMS 444.2 [M+H]⁺; ¹H NMR (400 MHz, DMSO-d₆) δ 10.06 (s, 1H), 9.75 (s, 1H), 7.50 (t, J=8.8 Hz, 1H), 7.09 (dd, J=2.9, 11.6 Hz, 1H), 6.88 (d, J=8.8 Hz, 1H), 6.73 (d, J=8.3 Hz, 1H), 4.68-4.52 (m, 2H), 3.16 (d, J=5.3 Hz, 1H), 2.09 (t, J=11.8 Hz, 1H), 1.89-1.62 (m, 4H), 1.45-1.25 (m, 11H), 1.20-0.97 (m, 2H).

### Step 2: Synthesis of trans-tert-butyl (4-(5-((4-chloro-3-fluorophenoxy)methyl)-1,3,4-oxadiazol-2-yl)cyclohexyl)carbamate

To a stirred mixture of trans-tert-butyl (4-(2-(2-(4-chloro-3-fluorophenoxy)acetyl)hydrazinecarbonyl)cyclohexyl)carbamate (600 mg, 1.354 mmol, 1 eq.), K₂CO₃ (936 mg, 6.78 mmol, 5.0 eq.), and molecular sieves (600 mg) in acetonitrile (15 mL) was added 4-toluenesulfonyl chloride (643 mg, 3.39 mmol, 2.5 eq.), and the resulting reaction mixture was stirred at 100° C. overnight. Product formation was confirmed by LCMS and TLC. Upon completion, the reaction mixture was filtered through Celite®. The filtrate was diluted with EtOAc (100 mL), washed with water (50 mL) and brine (50 mL), dried over anhydrous Na₂SO₄, and concentrated. The crude product was washed with pentane to obtain trans-tert-butyl (4-(5-((4-chloro-3-fluorophenoxy)methyl)-1,3,4-oxadiazol-2-yl)cyclohexyl)carbamate (400 mg, 69.6% yield) as an off white solid. LCMS 426.3 [M+H]⁺; ¹H NMR (400 MHz, DMSO-d₆) δ 7.53 (t, J=8.8 Hz, 1H), 7.24 (dd, J=2.9, 11.2 Hz, 1H), 6.96 (dd, J=1.8, 8.8 Hz, 1H), 6.83 (d, J=7.5 Hz, 1H), 5.41 (s, 2H), 3.24 (br. s., 1H), 2.87 (t, J=12.1 Hz, 1H), 2.06 (d, J=11.4 Hz, 2H), 1.86 (d, J=11.0 Hz, 2H), 1.64-1.49 (m, 2H), 1.38 (s, 9H), 1.32-1.11 (m, 2H).

### Step 3: Synthesis of trans-4-(5-((4-chloro-3-fluorophenoxy)methyl)-1,3,4-oxadiazol-2-yl)cyclohexan-1-amine 2,2,2-trifluoroacetate

To a stirred solution of trans-tert-butyl (4-(5-((4-chloro-3-fluorophenoxy)methyl)-1,3,4-oxadiazol-2-yl)cyclohexyl)carbamate (400 mg, 0.941 mmol, 1 eq.) in DCM (10 mL) was added trifluoroacetic acid (1.0 mL) and the resulting reaction mixture was stirred at RT overnight. Product formation was confirmed by LCMS. Upon completion, the reaction mixture was concentrated under reduced pressure. The crude product was crystallized in diethyl ether, dried under vacuum to obtain trans-4-(5-((4-chloro-3-fluorophenoxy)methyl)-1,3,4-oxadiazol-2-yl)cyclohexan-1-amine 2,2,2-trifluoroacetate (360 mg, 87.2% yield) as an off white solid. LCMS 326.2 [M+H]⁺; ¹H NMR (400 MHz, DMSO-d₆) δ 7.78 (br. s., 1H), 7.55 (d, J=9.6 Hz, 1H), 7.24 (d, J=11.0 Hz, 1H), 6.99 (d, J=19.7 Hz, 1H), 5.43 (s, 2H), 3.17 (br. s., 1H), 3.07 (br. s., 1H), 2.97 (br. s., 2H), 2.11 (br. s., 2H), 1.59 (d, J=12.7 Hz, 2H), 1.45 (br. s., 2H).

### Step 4: Synthesis of trans-2-(4-chloro-3-fluorophenoxy)-N-(4-(5-((4-chloro-3-fluorophenoxy)methyl)-1,3,4-oxadiazol-2-yl)cyclohexyl)acetamide

To a stirred solution of trans-4-(5-((4-chloro-3-fluorophenoxy)methyl)-1,3,4-oxadiazol-2-yl)cyclohexan-1-amine 2,2,2-trifluoroacetate (100 mg, 0.228 mmol, 1 eq.), 2-(4-chloro-3-fluorophenoxy)acetic acid (56 mg, 0.273 mmol, 1.2 eq.), and HATU (130 mg, 0.342 mmol, 1.5 eq.) in DMF (3.0 mL) was added DIPEA (0.2 mL, 0.9112 mmol, 4 eq.), and the resulting reaction mixture was stirred at RT overnight. Product formation was confirmed by LCMS. Upon completion, the reaction mixture was poured into ice cold water (100 ml). The resulting solid was filtered off and dried under vacuum. The crude product was purified by flash chromatography (2% MeOH in DCM as an eluent) to obtain trans-2-(4-chloro-3-fluorophenoxy)-N-(4-(5-((4-chloro-3-fluorophenoxy)methyl)-1,3,4-oxadiazol-2-yl)cyclohexyl)acetamide (Compound 13-84 mg, 72.2% yield) as a white solid. LCMS 512.4 [M+H]⁺; ¹H NMR (400 MHz, DMSO-d₆) δ 8.04 (d, J=8.3 Hz, 1H), 7.50 (t, J=8.8 Hz, 1H), 7.53 (t, J=8.8 Hz, 1H), 7.25 (dd, J=2.4, 11.2 Hz, 1H), 7.07 (dd, J=2.9, 11.2 Hz, 1H), 6.96 (d, J=9.2 Hz, 1H), 6.85 (d, J=9.2 Hz, 1H), 5.42 (s, 2H), 4.51 (s, 2H), 3.67 (d, J=7.5 Hz, 1H), 2.94 (t, J=11.6 Hz, 1H), 2.09 (d, J=12.3 Hz, 2H), 1.86 (d, J=10.1 Hz, 2H), 1.70-1.51 (m, 2H), 1.47-1.25 (m, 2H).

### Example 14

### Synthesis of trans-2-(4-chloro-3-fluorophenoxy)-N-(4-(5-(6-chloroquinolin-2-yl)-1,3,4-oxadiazol-2-yl)cyclohexyl)acetamide

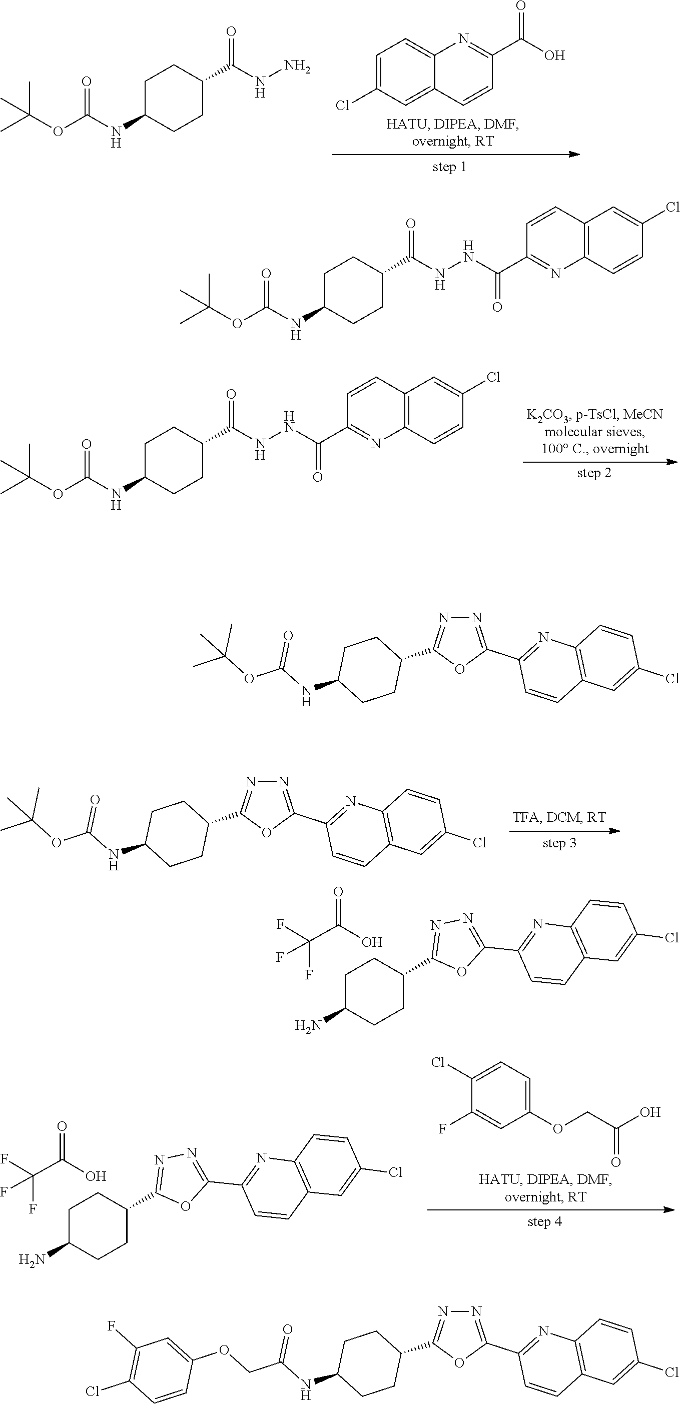

### Step 1: Synthesis of trans-tert-butyl (4-(2-(6-chloroquinoline-2-carbonyl)hydrazine-l-carbonyl)cyclohexyl)carbamate

To a stirred mixture of trans-tert-butyl (4-(hydrazinecarbonyl)cyclohexyl)carbamate (500 mg, 1.95 mmol, 1 eq.), 6-chloroquinoline-2-carboxylic acid (483 mg, 2.33 mmol, 1.2 eq.), and HATU (1108 mg, 2.92 mmol, 1.5 eq.) in DMF (5 mL) was added DIPEA (1.4 mL, 7.78 mmol, 4.0 eq.), and the resulting reaction mixture was stirred at RT overnight. Product formation was confirmed by LCMS. Upon completion, the reaction mixture was poured into ice cold water (100 ml). The resulting off white solid was filtered off and dried under vacuum to obtain trans-tert-butyl (4-(2-(6-chloroquinoline-2-carbonyl)hydrazine-1-carbonyl)cyclohexyl)carbamate (860 mg, 99.2% yield) as an off white solid. LCMS 447.3 [M+H]⁺; ¹H NMR (400 MHz, DMSO-d₆) δ 10.59 (s, 1H), 9.98 (s, 1H), 8.63-8.53 (m, 1H), 8.32-8.25 (m, 1H), 8.21-8.06 (m, 2H), 7.91 (dd, J=2.4, 9.0 Hz, 1H), 6.75 (d, J=7.9 Hz, 1H), 3.25-3.10 (m, 1H), 2.19 (t, J=12.1 Hz, 1H), 1.81 (d, J=11.4 Hz, 4H), 1.58-1.29 (m, 11H), 1.26-1.09 (m, 2H).

### Step 2: Synthesis of trans-tert-butyl (4-(5-(6-chloroquinolin-2-yl)-1,3,4-oxadiazol-2-yl)cyclohexyl)carbamate

To a stirred solution of trans-tert-butyl (4-(2-(6-chloroquinoline-2-carbonyl)hydrazine-1-carbonyl)cyclohexyl)carbamate (860 mg, 1.93 mmol, 1 eq.), K₂CO₃ (1330 mg, 9.64 mmol, 5.0 eq.) and molecular sieves (1000 mg) in acetonitrile (20 mL) was added 4-toluenesulfonyl chloride (915 mg, 4.82 mmol, 2.5 eq.) and the resulting reaction mixture was stirred at 100° C. overnight. Product formation was confirmed by LCMS and TLC. Upon completion, the reaction mixture was filtered through Celite®. The filtrate was diluted with EtOAc (100 mL). Organic layer was washed with water (50 mL) and brine (50 mL), dried over anhydrous Na₂SO₄ and concentrated. The crude product was washed with pentane to obtain trans-tert-butyl (4-(5-(6-chloroquinolin-2-yl)-1,3,4-oxadiazol-2-yl)cyclohexyl)carbamate (630 mg, 76.36% yield) as an off white solid. LCMS 429.3 [M+H]⁺; ¹H NMR (400 MHz, DMSO-d₆) δ 8.59 (d, J=8.3 Hz, 1H), 8.39-8.22 (m, 2H), 8.18 (d, J=8.8 Hz, 1H), 7.88 (dd, J=2.6, 9.2 Hz, 1H), 6.84 (br. s., 1H), 3.01 (br. s., 2H), 2.17 (d, J=11.8 Hz, 2H), 1.89 (br. s., 2H), 1.70-1.57 (m, 2H), 1.44-1.26 (m, 9H), 1.21 (br. s., 2H).

### Step 3: Synthesis of trans-4-(5-(6-chloroquinolin-2-yl)-1,3,4-oxadiazol-2-yl)cyclohexan-1-amine 2,2,2-trifluoroacetate

To a stirred solution of trans-tert-butyl (4-(5-(6-chloroquinolin-2-yl)-1,3,4-oxadiazol-2-yl)cyclohexyl)carbamate (630 mg, 1.47 mmol, 1 eq.) in DCM (10 mL) was added trifluoroacetic acid (1.5 mL), and the resulting reaction mixture was stirred at RT overnight. Product formation was confirmed by LCMS. Upon completion, the reaction mixture was concentrated under reduced pressure. The crude product was crystallized in diethyl ether and dried under vacuum to obtain trans-4-(5-(6-chloroquinolin-2-yl)-1,3,4-oxadiazol-2-yl)cyclohexan-1-amine 2,2,2-trifluoroacetate (600 mg, 92.3% yield) as an off white solid. LCMS 329.3 [M+H]⁺; ¹H NMR (400 MHz, DMSO-d₆) δ 8.61 (d, J=8.8 Hz, 1H), 8.37-8.21 (m, 2H), 8.17 (d, J=9.2 Hz, 1H), 7.97-7.83 (m, 3H), 3.20-3.08 (m, 2H), 2.31-2.19 (m, 2H), 2.07 (d, J=10.1 Hz, 2H), 1.79-1.61 (m, 2H), 1.61-1.44 (m, 2H).

### Step 4: Synthesis of trans-2-(4-chloro-3-fluorophenoxy)-N-(4-(5-(6-chloroquinolin-2-yl)-1,3,4-oxadiazol-2-yl)cyclohexyl)acetamide

To a stirred solution of trans-4-(5-(6-chloroquinolin-2-yl)-1,3,4-oxadiazol-2-yl)cyclohexan-1-amine 2,2,2-trifluoroacetate (100 mg, 0.23 mmol, 1 eq.), 2-(4-chloro-3-fluorophenoxy)acetic acid (46.2 mg, 0.23 mmol, 1 eq.), and HATU (133 mg, 0.345 mmol, 1.5 eq.) in DMF (3.0 mL) was added DIPEA (0.2 mL, 0.922 mmol, 4 eq.), and the resulting reaction mixture was stirred at RT overnight. Product formation was confirmed by LCMS. Upon completion, the reaction mixture was poured into ice cold water (100 ml). The resulting solid was filtered off and dried under vacuum. The crude product was crystallized in diethyl ether to obtain trans-2-(4-chloro-3-fluorophenoxy)-N-(4-(5-(6-chloroquinolin-2-yl)-1,3,4-oxadiazol-2-yl)cyclohexyl)acetamide (Compound 14-90 mg, 76.2% yield) as an off white solid. LCMS 515.5 [M+H]⁺; ¹H NMR (400 MHz, DMSO-d₆) δ 8.61 (d, J=8.8 Hz, 1H), 8.37-8.22 (m, 2H), 8.19 (d, J=8.8 Hz, 1H), 8.08 (d, J=7.9 Hz, 1H), 7.90 (dd, J=2.4, 9.0 Hz, 1H), 7.51 (t, J=9.0 Hz, 1H), 7.09 (dd, J=3.1, 11.4 Hz, 1H), 6.87 (d, J=7.0 Hz, 1H), 4.53 (s, 2H), 3.74 (br. s., 1H), 3.08 (d, J=11.8 Hz, 1H), 2.22 (d, J=13.2 Hz, 2H), 1.93 (d, J=9.2 Hz, 2H), 1.79-1.60 (m, 2H), 1.54-1.37 (m, 2H).

### Example 15

### Synthesis of trans-N-(4-(5-(4-chlorophenyl)-1,3,4-oxadiazol-2-yl)cyclohexyl)-5-(difluoromethyl)pyrazine-2-carboxamide

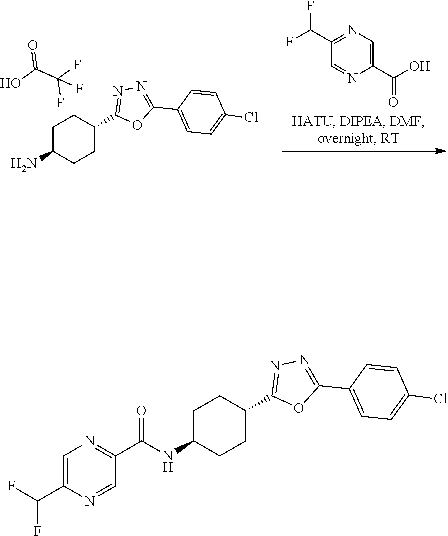

To a stirred solution of trans-4-(5-(4-chlorophenyl)-1,3,4-oxadiazol-2-yl)cyclohexan-1-amine 2,2,2-trifluoroacetate (60 mg, 0.256 mmol, 1 eq.), 5-(difluoromethyl)pyrazine-2-carboxylic acid (45 mg, 0.256 mmol, 1 eq.), and HATU (146 mg, 0.383 mmol, 1.5 eq.) in DMF (4 mL) was added DIPEA (0.2 mL, 1.022 mmol, 4 eq.), and the resulting reaction mixture was stirred at RT overnight. Product formation was confirmed by LCMS. Upon completion, the reaction mixture was poured into ice cold water (20 ml). The resulting solid was filtered off and dried under vacuum to obtain a crude solid which was crystallized in methanol to obtain trans-N-(4-(5-(4-chlorophenyl)-1,3,4-oxadiazol-2-yl)cyclohexyl)-5-(difluoromethyl)pyrazine-2-carboxamide (Compound 15-25 mg, 22.72%) as an off white solid. LCMS 434.4 [M+H]⁺; ¹H NMR (400 MHz, DMSO-d₆) δ 9.29 (s, 1H), 9.02 (s, 1H), 8.92 (d, J=8.3 Hz, 1H), 8.02 (d, J=8.3 Hz, 2H), 7.68 (d, J=8.3 Hz, 2H), 7.22 (s, 1H), 3.95 (br. s., 1H), 2.99 (br. s., 1H), 2.21 (br. s., 2H), 1.97 (d, J=9.6 Hz, 2H), 1.80-1.61 (m, 4H).

### Example 16

### Synthesis of trans-N-(4-(5-((4-chlorophenoxy)methyl)-1,3,4-oxadiazol-2-yl)cyclohexyl)-5-(difluoromethyl)pyrazine-2-carboxamide

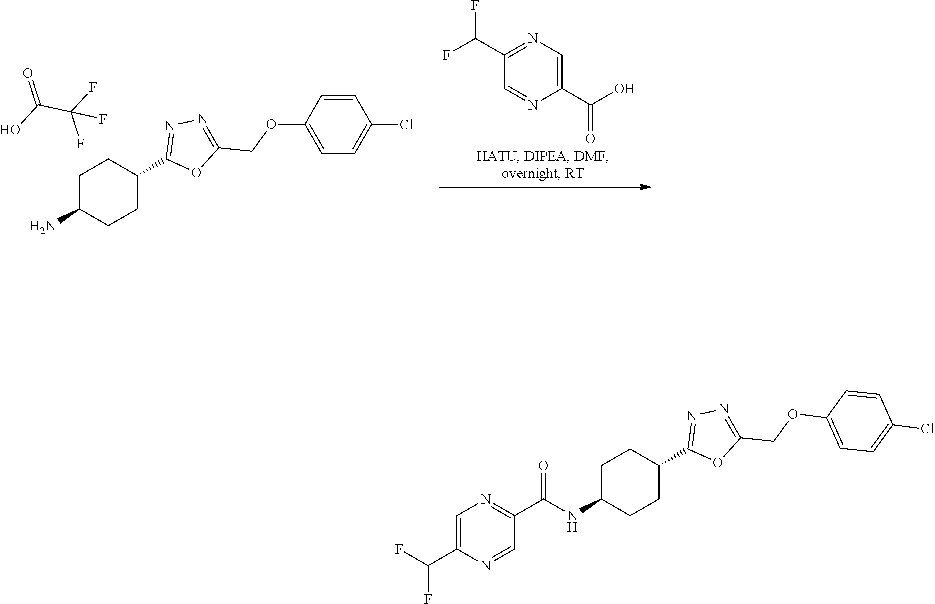

To a stirred solution of trans-4-(5-((4-chlorophenoxy)methyl)-1,3,4-oxadiazol-2-yl)cyclohexan-1-amine 2,2,2-trifluoroacetate (60 mg, 0.143 mmol, 1 eq.), 5-(difluoromethyl)pyrazine-2-carboxylic acid (25 mg, 0.143 mmol, 1 eq.), and HATU (81 mg, 0.214 mmol, 1.5 eq.) in DMF (2 mL) was added DIPEA (0.1 mL, 0.57 mmol, 4 eq.), and the resulting reaction mixture was stirred at RT overnight. Product formation was confirmed by LCMS. Upon completion, the reaction mixture was poured into ice cold water (50 ml). The resulting solid was filtered off and dried under vacuum. The crude product was crystallized in methanol to obtain trans-N-(4-(5-((4-chlorophenoxy)methyl)-1,3,4-oxadiazol-2-yl)cyclohexyl)-5-(difluoromethyl)pyrazine-2-carboxamide (Compound 16-10 mg, 24.42% yield) as an off white solid. LCMS 464.4 [M+H]⁺; ¹H NMR (400 MHz, DMSO-d₆) δ 9.27 (br. s., 1H), 9.01 (br. s., 1H), 8.90 (d, J=7.9 Hz, 1H), 7.38 (d, J=8.3 Hz, 2H), 7.22 (br. s., 1H), 7.10 (d, J=9.2 Hz, 2H), 5.39 (s, 2H), 3.89 (br. s., 1H), 2.94 (br. s., 1H), 2.13 (br. s., 2H), 1.94 (br. s., 2H), 1.63 (br. s., 4H).

### Example 17

### Synthesis of trans-2-(4-chloro-3-fluorophenoxy)-N-(4-(5-(4-chloro-3-fluorophenyl)-1,3,4-oxadiazol-2-yl cyclohexyl)acetamide

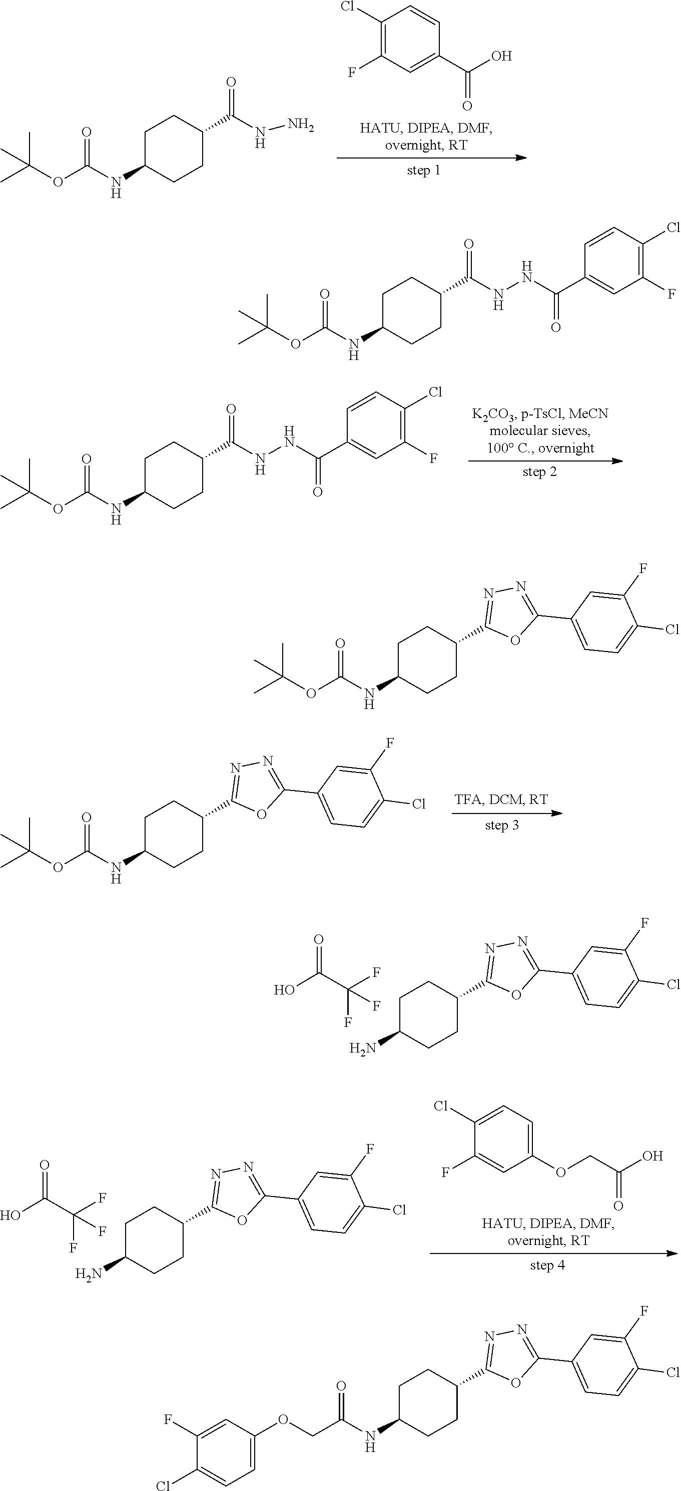

### Step 1: Synthesis of trans-tert-butyl (4-(2-(4-chloro-3-fluorobenzoyl)hydrazine-l-carbonyl)cyclohexyl)carbamate

To a stirred mixture of trans-tert-butyl (4-(hydrazinecarbonyl)cyclohexyl)carbamate (200 mg, 0.778 mmol, 1 eq.), 4-chloro-3-fluorobenzoic acid (136 mg, 0.778 mmol, 1 eq.), and HATU (443 mg, 1.17 mmol, 1 eq.) in DMF (4 mL) was added DIPEA (0.56 mL, 3.14 mmol, 4.0 eq.), and the resulting reaction mixture was stirred at RT overnight. Product formation was confirmed by LCMS. Upon completion, the reaction mixture was poured into ice cold water (100 ml). The resulting off white solid was filtered off and dried under vacuum to obtain trans-tert-butyl (4-(2-(4-chloro-3-fluorobenzoyl)hydrazine-1-carbonyl)cyclohexyl)carbamate (180 mg, 56.07% yield) as an off white solid. LCMS 414.3 [M+H]⁺; ¹H NMR (400 MHz, DMSO-d₆) δ 10.51 (br. s., 1H), 9.88 (br. s., 1H), 7.84 (d, J=9.6 Hz, 1H), 7.80-7.60 (m, 2H), 6.74 (d, J=7.9 Hz, 1H), 3.19 (br. s., 1H), 2.14 (d, J=11.4 Hz, 1H), 1.79 (d, J=13.6 Hz, 4H), 1.38 (s, 9H), 1.23-1.03 (m, 4H).

### Step 2: Synthesis of tert-butyl (4-(5-(4-chloro-3-fluorophenyl)-1,3,4-oxadiazol-2-yl)cyclohexyl)carbamate

To a stirred mixture of trans-tert-butyl (4-(2-(4-chloro-3-fluorobenzoyl)hydrazine-1-carbonyl)cyclohexyl)carbamate (180 mg, 0.436 mmol, 1 eq.), K₂CO₃ (300 mg, 2.18 mmol, 5.0 eq.), and molecular sieves (180 mg) in acetonitrile (5 mL) was added 4-toluenesulfonyl chloride (207 mg, 1.09 mmol, 2.5 eq.), and the resulting reaction mixture was stirred at 100° C. overnight. Product formation was confirmed by LCMS and TLC. Upon completion, the reaction mixture filtered through Celite®. The filtrate was diluted with EtOAc (100 mL), washed with water (50 mL) and brine (50 mL), dried over anhydrous Na₂SO₄, and concentrated. The crude product was washed with pentane to obtain trans-tert-butyl (4-(5-(4-chloro-3-fluorophenyl)-1,3,4-oxadiazol-2-yl)cyclohexyl)carbamate (140 mg, 81.4% yield) as off white solid. LCMS 396.3 [M+H]⁺; ¹H NMR (400 MHz, DMSO-d₆) δ 7.87-7.71 (m, 1H), 7.47 (d, J=7.9 Hz, 1H), 7.11 (d, J=8.3 Hz, 1H), 6.84 (d, J=7.5 Hz, 1H), 2.92 (t, J=12.1 Hz, 1H), 2.36-2.28 (m, 1H), 2.15 (d, J=13.2 Hz, 2H), 1.90 (d, J=12.3 Hz, 2H), 1.70-1.51 (m, 2H), 1.39 (s, 9H), 1.36-1.08 (m, 2H).

### Step 3: Synthesis of trans-4-(5-(4-chloro-3-fluorophenyl)-1,3,4-oxadiazol-2-yl)cyclohexan-1-amine 2,2,2-trifluoroacetate

To a stirred solution of trans-tert-butyl (4-(5-(4-chloro-3-fluorophenyl)-1,3,4-oxadiazol-2-yl)cyclohexyl)carbamate (140 mg, 0.354 mmol, 1 eq.) in DCM (10 mL) was added trifluoroacetic acid (0.5 mL), and the resulting reaction mixture was stirred at RT overnight under nitrogen atmosphere. Product formation was confirmed by LCMS. Upon completion, the reaction mixture was concentrated under reduced pressure. The crude product was crystallized in diethyl ether and dried under vacuum to obtain trans-4-(5-(4-chloro-3-fluorophenyl)-1,3,4-oxadiazol-2-yl)cyclohexan-1-amine 2,2,2-trifluoroacetate (120 mg, 82.9% yield) as an off white solid. LCMS 296.3 [M+H]⁺; ¹H NMR (400 MHz, DMSO-d₆) δ 7.99 (d, J=8.8 Hz, 1H), 7.90-7.70 (m, 2H), 7.47 (d, J=7.9 Hz, 1H), 7.11 (d, J=8.3 Hz, 1H), 3.14-2.98 (m, 2H), 2.22 (d, J=11.0 Hz, 2H), 2.11-1.98 (m, 2H), 1.67 (q, J=12.9 Hz, 2H), 1.57-1.35 (m, 2H).

### Step 4: Synthesis of trans-2-(4-chloro-3-fluorophenoxy)-N-(4-(5-(4-chloro-3-fluorophenyl)-1,3,4-oxadiazol-2-yl)cyclohexyl)acetamide

To a stirred solution of trans-4-(5-(4-chloro-3-fluorophenyl)-1,3,4-oxadiazol-2-yl)cyclohexan-1-amine 2,2,2-trifluoroacetate (60 mg, 0.147 mmol, 1 eq.), 2-(4-chloro-3-fluorophenoxy)acetic acid (30 mg, 0.147 mmol, 1 eq.), and HATU (84 mg, 0.22 mmol, 1.5 eq.) in DMF (1 mL) was added DIPEA (0.1 mL, 0.586 mmol, 4 eq.), and the resulting reaction mixture was stirred at RT overnight. Product formation was confirmed by LCMS. Upon completion, the reaction mixture was poured into ice cold water (50 ml). The resulting solid was filtered off and dried under vacuum. The crude product was crystallized in methanol to obtain trans-2-(4-chloro-3-fluorophenoxy)-N-(4-(5-(4-chloro-3-fluorophenyl)-1,3,4-oxadiazol-2-yl)cyclohexyl)acetamide (Compound 17-60 mg, 85% yield) as a white solid. LCMS 482.4 [M+H]⁺; ¹H NMR (400 MHz, DMSO-d₆) δ 8.06 (d, J=7.5 Hz, 1H), 8.01 (d, J=9.6 Hz, 1H), 7.85 (br. s., 2H), 7.53-7.48 (t, J=8.6 Hz, 1H), 7.06-7.09 (s, 1H), 6.86 (d, J=7.5 Hz, 1H), 4.52 (s, 2H), 3.71 (br. s., 1H), 2.99 (br. s., 1H), 2.20-2.17 (s, 2H), 1.91-1.89 (s, 2H), 1.68-1.62 (m, 2H), 1.46-1.43 (s, 2H).

### Example 18

### Synthesis of trans-N-(4-(5-(6-chloro-3,4-dihydro-2H-benzo[b][1,4]oxazin-2-yl)-1,3,4-oxadiazol-2-yl)cyclohexyl)-2-(4-chloro-3-fluorophenoxy)acetamide

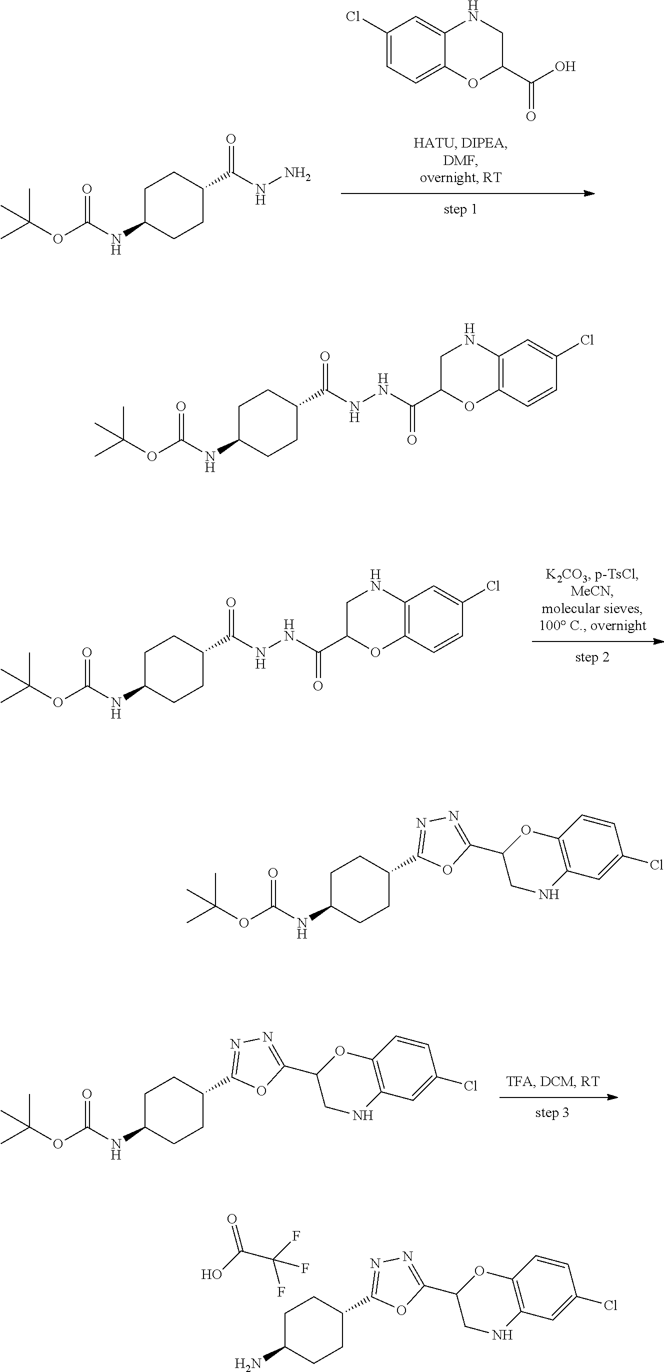

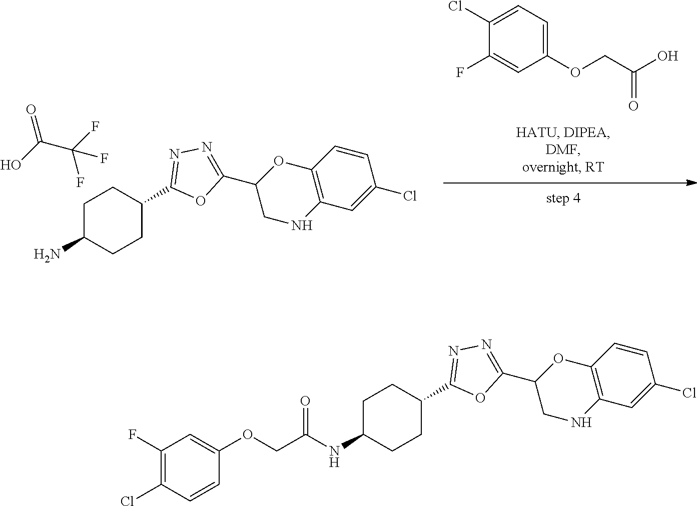

### Step 1: Synthesis of trans-tert-butyl 4-(2-(6-chloro-3,4-dihydro-2H-benzo[b][1,4]oxazine-2-carbonyl)hydrazine-1-carbonyl)cyclohexylcarbamate

To a stirred mixture of trans-tert-butyl (4-(hydrazinecarbonyl)cyclohexyl)carbamate (200 mg, 0.778 mmol, 1 eq.), 6-chloro-3,4-dihydro-2H-benzo[b][1,4]oxazine-2-carboxylic acid (166 mg, 0.778 mmol, 1.1 eq.), and HATU (443 mg, 1.17 mmol, 1.5 eq.) in DMF (4 mL) was added DIPEA (0.56 mL, 3.11 mmol, 4.0 eq.), and the resulting reaction mixture was stirred at RT overnight. Product formation was confirmed by LCMS. Upon completion, the reaction mixture was poured into ice cold water (50 ml). The resulting off white solid was filtered off and dried under vacuum to obtain trans-tert-butyl 4-(2-(6-chloro-3,4-dihydro-2H-benzo[b][1,4]oxazine-2-carbonyl)hydrazine-1-carbonyl)cyclohexylcarbamate (240 mg, 68.3% yield) as an off white solid. LCMS 453.4 [M+H]⁺; ¹H NMR (400 MHz, DMSO-d₆) δ 9.93 (br. s., 1H), 9.74 (br. s., 1H), 6.82-6.66 (m, 2H), 6.60 (d, J=2.6 Hz, 1H), 6.51 (dd, J=2.4, 8.6 Hz, 1H), 6.20 (br. s., 1H), 4.60 (br. s., 1H), 3.43 (d, J=12.3 Hz, 1H), 3.16 (br. s., 1H), 2.08 (br. s., 1H), 1.90-1.61 (m, 4H), 1.37 (s, 9H), 1.20-1.03 (m, 2H).

### Step 2: Synthesis of trans-tert-butyl (4-(5-(6-chloro-3,4-dihydro-2H-benzo[b][1,4]oxazin-2-yl)-1,3,4-oxadiazol-2-yl)cyclohexyl)carbamate

To a stirred solution of trans-tert-butyl 4-(2-(6-chloro-3,4-dihydro-2H-benzo[b][1,4]oxazine-2-carbonyl)hydrazine-1-carbonyl)cyclohexylcarbamate (240 mg, 0.531 mmol, 1 eq.), K₂CO₃ (366 mg, 2.65 mmol, 5.0 eq.), and molecular sieves (200 mg) in acetonitrile (5 mL) was added 4-toluenesulfonyl chloride (252 mg, 1.328 mmol, 2.5 eq.), and the resulting reaction mixture was heated at 100° C. overnight. Product formation was confirmed by LCMS and TLC. Upon completion, the reaction mixture was filtered through Celite®. The filtrate was diluted with EtOAc (50 mL), washed with water (50 mL) and brine (50 mL), dried over anhydrous Na₂SO₄, and concentrated. The crude product was washed with pentane to obtain trans-tert-butyl (4-(5-(6-chloro-3,4-dihydro-2H-benzo[b][1,4]oxazin-2-yl)-1,3,4-oxadiazol-2-yl)cyclohexyl)carbamate (200 mg, 87% yield) as an off white solid. LCMS 435.3 [M+H]⁺; ¹H NMR (400 MHz, DMSO-d₆) δ 6.89-6.76 (m, 1H), 6.64 (d, J=2.2 Hz, 1H), 6.51 (dd, J=2.4, 8.6 Hz, 1H), 6.37 (br. s., 1H), 5.58-5.41 (m, 1H), 3.68 (br. s., 1H), 3.61-3.49 (m, 1H), 2.87 (d, J=13.2 Hz, 1H), 2.78 (br. s., 2H), 2.05 (d, J=14.9 Hz, 2H), 1.86 (d, J=11.8 Hz, 2H), 1.51 (d, J=12.7 Hz, 2H), 1.38 (s, 9H), 1.35-1.20 (m, 2H).

### Step 3: Synthesis of trans-4-(5-(6-chloro-3,4-dihydro-2H-benzo[b][1,4]oxazin-2-yl)-1,3,4-oxadiazol-2-yl)cyclohexanamine 2,2,2-trifluoroacetate

To a stirred solution of trans-tert-butyl (4-(5-(6-chloro-3,4-dihydro-2H-benzo[b][1,4]oxazin-2-yl)-1,3,4-oxadiazol-2-yl)cyclohexyl)carbamate (200 mg, 0.459 mmol, 1 eq.) in DCM (10 mL) was added trifluoroacetic acid (0.5 mL) and the resulting reaction mixture was stirred at RT overnight. Product formation was confirmed by LCMS. Upon completion, the reaction mixture was concentrated under reduced pressure. The crude product was crystallized in diethyl ether and dried under vacuum to obtain trans-4-(5-(6-chloro-3,4-dihydro-2H-benzo[b][1,4]oxazin-2-yl)-1,3,4-oxadiazol-2-yl)cyclohexanamine 2,2,2-trifluoroacetate (200 mg, 97.6% yield) as an off white solid. LCMS 335.1 [M+H]⁺; ¹H NMR (400 MHz, DMSO-d₆) δ 7.82 (br. s., 2H), 7.47 (d, J=7.9 Hz, 1H), 7.11 (d, J=7.5 Hz, 1H), 6.76 (d, J=8.8 Hz, 1H), 6.64 (d, J=2.2 Hz, 1H), 6.51 (dd, J=2.6, 8.3 Hz, 1H), 5.50 (dd, J=3.1, 6.1 Hz, 1H), 3.67 (dd, J=2.9, 12.9 Hz, 1H), 3.56 (dd, J=6.1, 12.7 Hz, 1H), 3.07 (br. s., 1H), 2.97 (d, J=11.4 Hz, 2H), 2.11 (d, J=11.8 Hz, 2H), 2.00 (d, J=10.1 Hz, 1H), 1.64-1.50 (m, 2H), 1.48-1.19 (m, 2H).

### Step 4: Synthesis of trans-N-(4-(5-(6-chloro-3,4-dihydro-2H-benzo[b][1,4]oxazin-2-yl)-1,3,4-oxadiazol-2-yl)cyclohexyl)-2-(4-chloro-3-fluorophenoxy)acetamide

To a stirred solution of trans-4-(5-(6-chloro-3,4-dihydro-2H-benzo[b][1,4]oxazin-2-yl)-1,3,4-oxadiazol-2-yl)cyclohexanamine 2,2,2-trifluoroacetate (100 mg, 0.223 mmol, 1 eq.), 2-(4-chloro-3-fluorophenoxy)acetic acid (45.4 mg, 0.223 mmol, 1 eq.), and HATU (127 mg, 0.345 mmol, 1.5 eq.) in DMF (2.0 mL) was added DIPEA (0.2 mL, 0.892 mmol, 4 eq.), and the resulting reaction mixture was stirred at RT overnight. Product formation was confirmed by LCMS. Upon completion, the reaction mixture was poured into ice cold water (50 ml). The resulting solid was filtered off and dried under vacuum. The crude product was purified by reverse phase HPLC to obtain trans-N-(4-(5-(6-chloro-3,4-dihydro-2H-benzo[b][1,4]oxazin-2-yl)-1,3,4-oxadiazol-2-yl)cyclohexyl)-2-(4-chloro-3-fluorophenoxy)acetamide (Compound 18-20 mg, 17.25% yield) as a white solid. LCMS 512.4 [M+H]⁺; ¹H NMR (400 MHz, DMSO-d₆) δ 8.06 (d, J=7.9 Hz, 1H), 7.50 (t, J=8.8 Hz, 1H), 7.10-7.04 (m, 1H), 6.85 (d, J=7.0 Hz, 1H), 6.77 (d, J=8.8 Hz, 1H), 6.64 (d, J=2.2 Hz, 1H), 6.51 (d, J=6.1 Hz, 1H), 6.38 (br. s., 1H), 5.50 (d, J=3.1 Hz, 1H), 4.51 (s, 2H), 3.65 (s, 2H), 3.57 (br. s., 1H), 2.93 (br.s., 1H) 2.09 (d, J=9.6 Hz, 2H), 1.86 (d, J=9.6 Hz, 2H), 1.59-1.50 (m, 2H), 1.47-1.35 (m, 2H).

### Example 19

### Synthesis of trans-4-(5-(4-chlorophenyl)-1,3,4-oxadiazol-2-yl)-N-(6-chloroquinolin-2-yl)cyclohexane-1-carboxamide

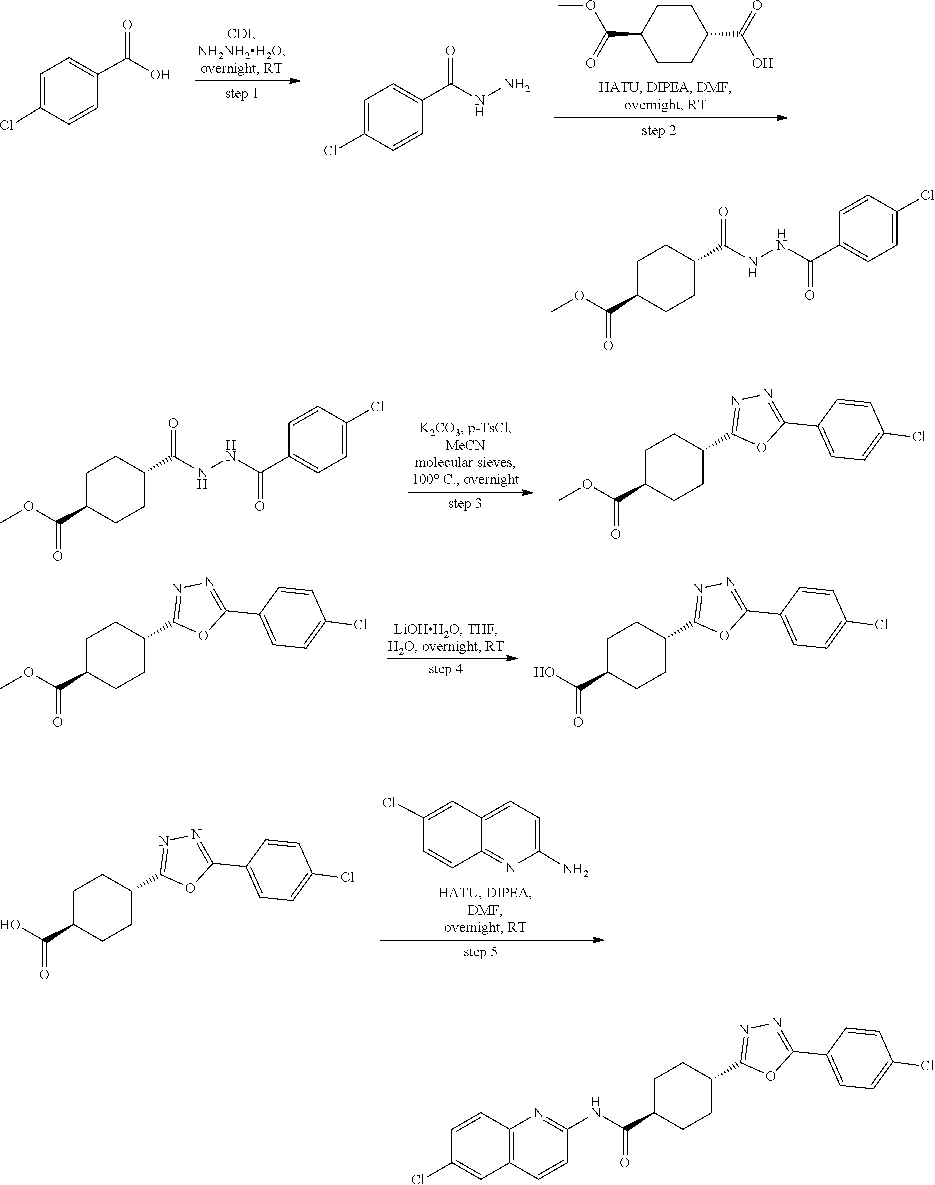

### Step 1: Synthesis of 4-chlorobenzohydrazide

To a stirred solution of 4-chlorobenzoic acid (1.00 g, 6.410 mmol, 1.0 eq.) in THE (7 mL) was added CDI (1.5 g, 9.615 mmol, 1.5 eq.) and the resulting reaction mixture was stirred at RT for 2 hours. Hydrazine hydrate (0.45 mL, 9.615 mmol, 1.5 eq.) was added and the reaction mixture was stirred overnight at RT. Product formation was confirmed by LCMS. Upon completion, the reaction mixture was concentrated under reduced pressure. The resulting residue was washed with diethyl ether and dried under vacuum to obtain 4-chlorobenzohydrazide (1.0 g, 91.82% yield) as a white solid. LCMS 170.9 [M+H]⁺; ¹H NMR (400 MHz, DMSO-d₆) δ 9.86 (br. s., 1H) 7.83 (m, J=8.77 Hz, 2H) 7.43-7.59 (m, 2H) 4.52 (br. s., 2H).

### Step 2: Synthesis of trans-methyl 4-(2-(4-chlorobenzoyl)hydrazine-1-carbonyl)cyclohexanecarboxylate

To a stirred solution of trans-4-(methoxycarbonyl)cyclohexane-1-carboxylic acid (200 mg, 1.07 mmol, 1.0 eq.), 4-chlorobenzohydrazide (219 mg, 1.29 mmol, 1.2 eq.) and HATU (813 mg, 2.14 mmol, 2.0 eq.) in DMF (1 mL) was added DIPEA (0.36 mL, 2.14 mmol, 2.0 eq.), and the resulting reaction mixture was stirred at RT overnight. Product formation was confirmed by LCMS. Upon completion, the reaction mixture was poured into ice cold water (100 ml). The resulting solid was filtered off and dried under vacuum to obtain trans-methyl 4-(2-(4-chlorobenzoyl)hydrazine-1-carbonyl)cyclohexanecarboxylate (250 mg, 68.8% yield) as an off white solid. LCMS 339.2 [M+H]⁺.

### Step 3: Synthesis of trans-methyl 4-(5-(4-chlorophenyl)-1,3,4-oxadiazol-2-yl)cyclohexanecarboxylate

To a stirred mixture of trans-methyl 4-(2-(4-chlorobenzoyl)hydrazine-1-carbonyl)cyclohexane-1-carboxylate (250 mg, 0.739 mmol, 1.0 eq.), K₂CO₃ (306 mg, 2.218 mmol, 3.0 eq.), and molecular sieves (250 mg) in acetonitrile (10 mL) was added 4-toluenesulfonyl chloride (351 mg, 1.847 mmol, 1.0 eq.), and the resulting reaction mixture was heated at 100° C. overnight. Product formation was confirmed by LCMS and TLC. Upon completion, the reaction mixture was filtered through Celite®. The filtrate was diluted with EtOAc (50 mL), washed with water (20 mL) and brine (20 mL), dried over anhydrous Na₂SO₄, and concentrated. The crude solid was purified by flash chromatography (0-50% ethyl acetate in hexane as an eluent) to obtain trans-methyl 4-(5-(4-chlorophenyl)-1,3,4-oxadiazol-2-yl)cyclohexane-1-carboxylate (140 mg, 59.3% yield) as an off white solid. LCMS 321.0 [M+H]⁺.

### Step 4: Synthesis of trans-4-(5-(4-chlorophenyl)-1,3,4-oxadiazol-2-yl)cyclohexane-1-carboxylic acid

To a stirred solution of trans-methyl 4-(5-(4-chlorophenyl)-1,3,4-oxadiazol-2-yl)cyclohexane-1-carboxylate (140 mg, 0.437 mmol, 1.0 eq.) in THE (3 mL) was added LiOH.H₂O (27 mg, 0.656 mmol, 1.5 eq.) and H₂O (3 ml), and the resulting reaction mixture was stirred at RT overnight. Product formation was confirmed by LCMS. Upon completion, the reaction mixture was concentrated under reduced pressure. The resulting residue was dissolved in water (10 mL) and acidified with 1 M HCl. The resulting solid was filtered off and dried under vacuum to obtain trans-4-(5-(4-chlorophenyl)-1,3,4-oxadiazol-2-yl)cyclohexane-1-carboxylic acid (110 mg, 82.7% yield) as a white solid. LCMS 307.0 [M+H]⁺.

### Step 5: Synthesis of trans-4-(5-(4-chlorophenyl)-1,3,4-oxadiazol-2-yl)-N-(6-chloroquinolin-2-yl)cyclohexane-1-carboxamide

To a stirred solution of trans-4-(5-(4-chlorophenyl)-1,3,4-oxadiazol-2-yl)cyclohexane-1-carboxylic acid (110 mg, 0.359 mmol, 1 eq.), 6-chloroquinolin-2-amine (76 mg, 0.431 mmol, 1.2 eq.), and HATU (272 mg, 0.718 mmol, 2.0 eq.) in DMF (0.5 mL) was added DIPEA (0.12 mL, 0.718 mmol, 2.0 eq.), and the resulting reaction mixture was stirred at RT overnight. Product formation was confirmed by LCMS. Upon completion, the reaction mixture was poured into ice cold water (10 ml). The resulting crude solid was filtered off and purified by flash chromatography (0-5% MeOH in DCM as an eluent). The product was then crystallized in DCM and dried under vacuum to obtain trans-4-(5-(4-chlorophenyl)-1,3,4-oxadiazol-2-yl)-N-(6-chloroquinolin-2-yl)cyclohexane-1-carboxamide (Compound 19-13 mg, 7.78% yield) as a white solid. LCMS 467.3[M+H]⁺; ¹H NMR (400 MHz, DMSO-d₆) δ 10.89 (s, 1H) 8.28-8.44 (m, 2H) 8.05 (d, J=2.19 Hz, 1H) 8.02 (d, J=8.77 Hz, 2H) 7.81 (d, J=8.77 Hz, 1H) 7.56-7.78 (m, 3H) 3.07 (br. s., 1H) 2.67 (br. s., 1H) 2.25 (br. s., 2H) 2.02 (br. s., 2H) 1.49-1.76 (m, 4H).

### Example 20

### Synthesis of trans-N-((4-(5-(4-chlorophenyl)-1,3,4-oxadiazol-2-yl)cyclohexyl)methyl)-5-(trifluoromethyl)picolinamide

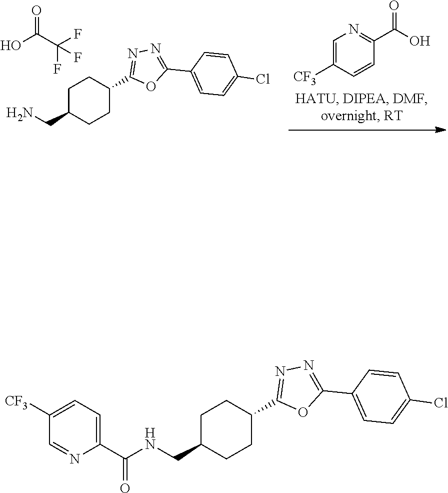

To a stirred solution of trans-(4-(5-(4-chlorophenyl)-1,3,4-oxadiazol-2-yl)cyclohexyl)methanamine 2,2,2-trifluoroacetate (70 mg, 0.173 mmol, 1 eq.), 5-(trifluoromethyl)picolinic acid (40 mg, 0.207 mmol, 1.2 eq.), and HATU (98 mg, 0.259 mmol, 1.5 eq.) in DMF (2 mL) was added DIPEA (0.2 mL, 0.691 mmol, 4 eq.), and the resulting reaction mixture was stirred at RT overnight. Product formation was confirmed by LCMS. Upon completion, the reaction mixture was poured into ice cold water (10 ml) and extracted with ethyl acetate (2×20 mL). The organic layers were washed with water (4×10 mL) and brine (1×10 mL), dried over anhydrous Na₂SO₄, and concentrated under reduced pressure. The crude product was purified by flash chromatography (0-5% MeOH in DCM as an eluent) to obtain trans-N-((4-(5-(4-chlorophenyl)-1,3,4-oxadiazol-2-yl)cyclohexyl)methyl)-5-(trifluoromethyl)picolinamide (Compound 20-26 mg, 32.5% yield) as a white solid. LCMS 465.2 [M+H]⁺; ¹H NMR (400 MHz, DMSO-d₆) δ 9.04 (br. s., 2H), 8.43 (d, J=8.3 Hz, 1H), 8.23 (d, J=8.3 Hz, 1H), 8.06-7.91 (m, 2H), 7.70-7.55 (m, 2H), 3.24 (t, J=6.4 Hz, 2H), 2.98 (t, J=12.1 Hz, 1H), 2.16 (d, J=10.1 Hz, 2H), 1.85 (d, J=13.2 Hz, 2H), 1.69 (br. s., 1H), 1.61-1.48 (m, 2H), 1.19-1.04 (m, 2H).

### Example 21

### Synthesis of trans-N-((4-(5-(4-chlorophenyl)-1,3,4-oxadiazol-2-yl)cyclohexyl)methyl)-6-(trifluoromethyl)nicotinamide

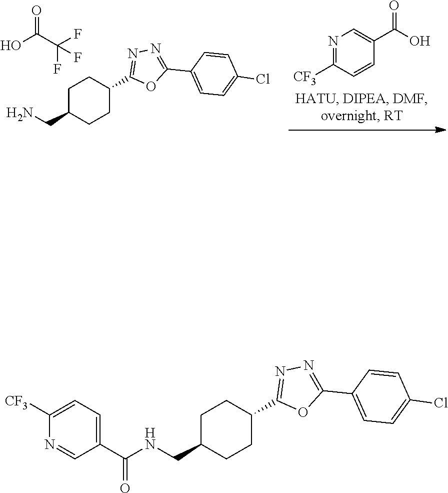

To a stirred solution of trans-(4-(5-(4-chlorophenyl)-1,3,4-oxadiazol-2-yl)cyclohexyl)methanamine 2,2,2-trifluoroacetate (70 mg, 0.173 mmol, 1 eq.), 6-(trifluoromethyl)nicotinic acid (40 mg, 0.207 mmol, 1.2 eq.) and HATU (98 mg, 0.259 mmol, 1.5 eq.) in DMF (2 mL) was added DIPEA (0.2 mL, 0.691 mmol, 4 eq.), and the resulting reaction mixture was stirred at RT overnight. Product formation was confirmed by LCMS. Upon completion, the reaction mixture was poured into ice cold water (10 ml) and extracted with ethyl acetate (2×20 mL). The organic layers were washed with water (4×10 mL) and brine (1×10 mL), dried over anhydrous Na₂SO₄, and concentrated under reduced pressure. The crude product was purified by flash chromatography (0-5% MeOH in DCM as an eluent) to obtain trans-N-((4-(5-(4-chlorophenyl)-1,3,4-oxadiazol-2-yl)cyclohexyl)methyl)-6-(trifluoromethyl)nicotinamide (Compound 21-20 mg, 25% yield) as a white solid. LCMS 465.2 [M+H]⁺; ¹H NMR (400 MHz, DMSO-d₆) δ 9.15 (s, 1H), 8.91 (br. s., 1H), 8.46 (d, J=8.3 Hz, 1H), 8.12-8.01 (m, 1H), 7.99 (s, 2H), 7.67 (d, J=8.8 Hz, 2H), 3.22 (t, J=6.4 Hz, 2H), 2.99 (t, J=12.1 Hz, 1H), 2.18 (d, J=11.0 Hz, 2H), 1.90 (d, J=11.8 Hz, 2H), 1.65 (br. s., 1H), 1.60-1.40 (m, 2H), 1.25-1.07 (m, 2H).

### Example 22

### Synthesis of trans-2-(4-chloro-3-fluorophenoxy)-N-(4-(5-(4-chlorophenyl)-1,3,4-thiadiazol-2-yl)cyclohexyl)acetamide

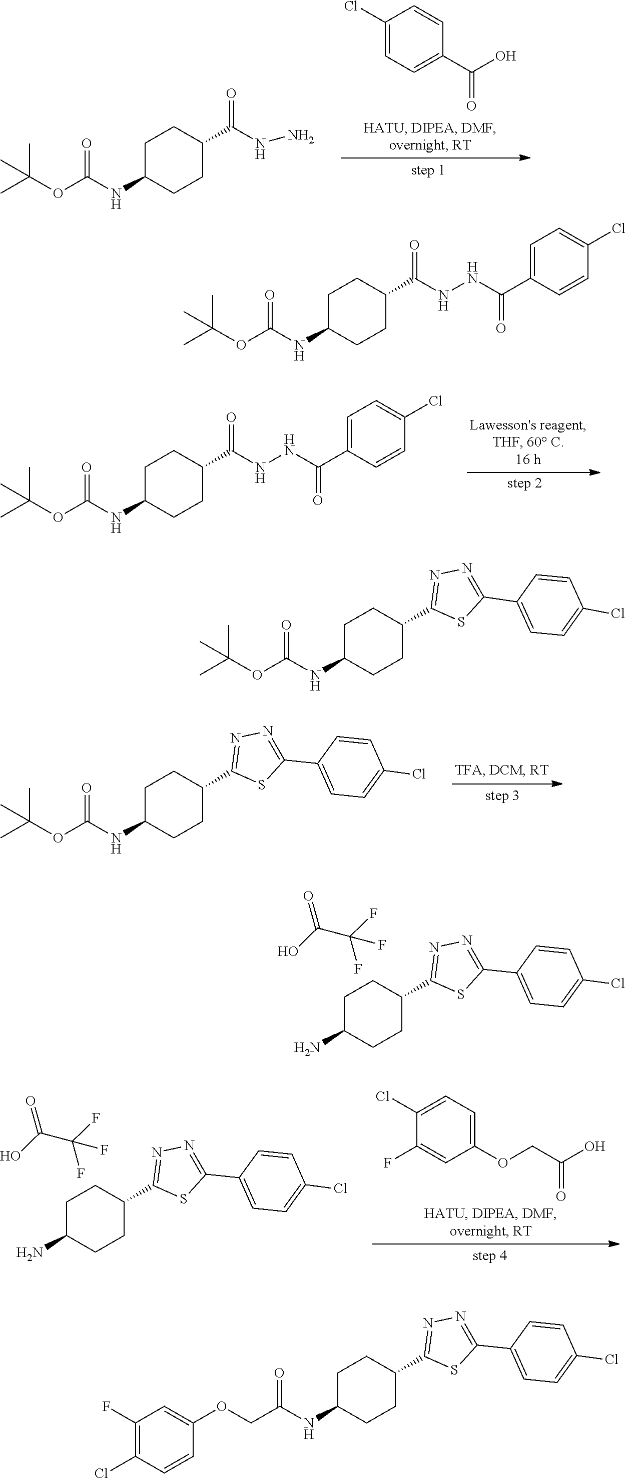

### Step 1: Synthesis of trans-tert-butyl (4-(2-(4-chlorobenzoyl)hydrazine-l-carbonyl)cyclohexyl)carbamate

To a stirred mixture of trans-tert-butyl (4-(hydrazinecarbonyl)cyclohexyl)carbamate (200 mg, 0.778 mmol, 1 eq.), 4-chlorobenzoic acid (134 mg, 0.856 mmol, 1.1 eq.), and HATU (444 mg, 1.17 mmol, 1.5 eq.) in DMF (5 mL) was added DIPEA (0.5 mL, 2.33 mmol, 3.0 eq.), and the resulting reaction mixture was stirred at RT overnight. Product formation was confirmed by LCMS. Upon completion, the reaction mixture was poured into ice cold water (50 ml). The resulting solid was filtered off and dried under vacuum to obtain trans-tert-butyl (4-(2-(4-chlorobenzoyl)hydrazine-1-carbonyl)cyclohexyl)carbamate (250 mg, 81% yield) as an off white solid. LCMS 396.3 [M+H]⁺; ¹H NMR (400 MHz, DMSO-d₆) δ 10.39 (s, 1H) 9.82 (s, 1H) 7.87 (m, J=8.33 Hz, 2H) 7.57 (m, J=8.33 Hz, 2H) 6.75 (d, J=7.89 Hz, 1H) 3.17 (br. s., 1H) 2.15 (t, J=11.84 Hz, 1H) 1.80 (t, J=13.37 Hz, 4H) 1.43-1.51 (m, 1H) 1.30-1.43 (m, 8H) 1.07-1.24 (m, 2H).

### Step 2: Synthesis of trans-tert-butyl (4-(5-(4-chlorophenyl)-1,3,4-thiadiazol-2-yl)cyclohexyl)carbamate

To a stirred solution of trans-tert-butyl (4-(2-(4-chlorobenzoyl)hydrazine-1-carbonyl)cyclohexyl)carbamate (80 mg, 0.202 mmol, 1 eq.) in THE (5 mL) was added Lawesson's reagent (106 mg, 0.262 mmol, 1.3 eq.). The resulting reaction mixture was heated at 60° C. for 16 h. Product formation was confirmed by LCMS and TLC. Upon completion, the reaction mixture was poured in water and extracted with ethyl acetate (25 mL×3). The combined organic layers were washed with brine solution (25 mL), dried over anhydrous Na₂SO₄, and concentrated under reduced pressure. The crude product was purified by flash chromatography (0-30% ethyl acetate in hexane as an eluent) to obtain trans-tert-butyl (4-(5-(4-chlorophenyl)-1,3,4-thiadiazol-2-yl)cyclohexyl)carbamate (50 mg, 62% yield) as an off white solid. LCMS 394.3 [M+H]⁺; ¹H NMR (400 MHz, DMSO-d₆) δ 7.96 (m, J=8.77 Hz, 2H) 7.62 (m, J=8.33 Hz, 2H) 6.84 (d, J=7.02 Hz, 1H) 3.12 (t, J=11.84 Hz, 1H) 2.14 (d, J=11.84 Hz, 2H) 1.90 (d, J=12.72 Hz, 2H) 1.54-1.70 (m, 3H) 1.39 (s, 9H) 1.26-1.36 (m, 2H).

### Step 3: Synthesis of trans-4-(5-(4-chlorophenyl)-1,3,4-thiadiazol-2-yl)cyclohexan-1-amine 2,2,2-trifluoroacetate

To a stirred solution of trans-tert-butyl (4-(5-(4-chlorophenyl)-1,3,4-thiadiazol-2-yl)cyclohexyl)carbamate (50 mg, 0.127 mmol, 1 eq.) in DCM (5 mL) was added trifluoroacetic acid (0.1 mL), and the resulting reaction mixture was stirred at RT overnight. Product formation was confirmed by LCMS. Upon completion, the reaction mixture was concentrated under reduced pressure. The crude product was crystallized in diethyl ether and dried under vacuum to obtain trans-4-(5-(4-chlorophenyl)-1,3,4-thiadiazol-2-yl)cyclohexan-1-amine 2,2,2-trifluoroacetate (45 mg, 88% yield) as an off white solid. LCMS 294.2 [M+H]⁺; ¹H NMR (400 MHz, DMSO-d₆) δ 7.91-8.04 (m, 2H) 7.83 (br. s., 2H) 7.54-7.67 (m, 2H) 3.05-3.27 (m, 3H) 2.20 (d, J=11.84 Hz, 2H) 2.05 (d, J=10.52 Hz, 2H) 1.59-1.75 (m, 2H) 1.36-1.58 (m, 2H).

### Step 4: Synthesis of trans-2-(4-chloro-3-fluorophenoxy)-N-(4-(5-(4-chlorophenyl)-1,3,4-thiadiazol-2-yl)cyclohexyl)acetamide

To a stirred solution of trans-4-(5-(4-chlorophenyl)-1,3,4-thiadiazol-2-yl)cyclohexan-1-amine 2,2,2-trifluoroacetate (45 mg, 0.114 mmol, 1 eq.), 2-(4-chloro-3-fluorophenoxy)acetic acid (26 mg, 0.125 mmol, 1.1 eq.), and HATU (66 mg, 0.171 mmol, 1.5 eq.) in DMF (2.0 mL) was added DIPEA (0.1 mL, 0.343 mmol, 3.0 eq.), and the resulting reaction mixture was stirred at RT overnight. Product formation was confirmed by LCMS. Upon completion, the reaction mixture was poured into ice cold water (50 ml). The resulting solid was filtered off and dried under vacuum. The crude product was purified by flash chromatography (0-30% MeOH in DCM as an eluent) to obtain trans-2-(4-chloro-3-fluorophenoxy)-N-(4-(5-(4-chlorophenyl)-1,3,4-thiadiazol-2-yl)cyclohexyl)acetamide (Compound 22-10 mg, 18% yield) as an off white solid. LCMS 480.4 [M+H]⁺; ¹H NMR (400 MHz, DMSO-d₆) δ 8.07 (d, J=7.89 Hz, 1H) 7.97 (d, J=8.77 Hz, 1H) 7.62 (d, J=8.77 Hz, 1H) 7.50 (t, J=8.99 Hz, 1H) 7.08 (dd, J=11.40, 2.63 Hz, 1H) 6.86 (d, J=9.21 Hz, 1H) 4.52 (s, 2H) 3.72 (br. s., 1H) 3.11-3.22 (m, 1H) 2.17 (d, J=10.96 Hz, 2H) 1.90 (d, J=10.96 Hz, 2H) 1.61-1.74 (m, 2H) 1.29-1.57 (m, 2H).

### Example 23

### Synthesis of trans-2-(4-chloro-3-fluorophenoxy)-N-(4-(4-(4-chlorophenyl)oxazol-2-yl)cyclohexyl)acetamide

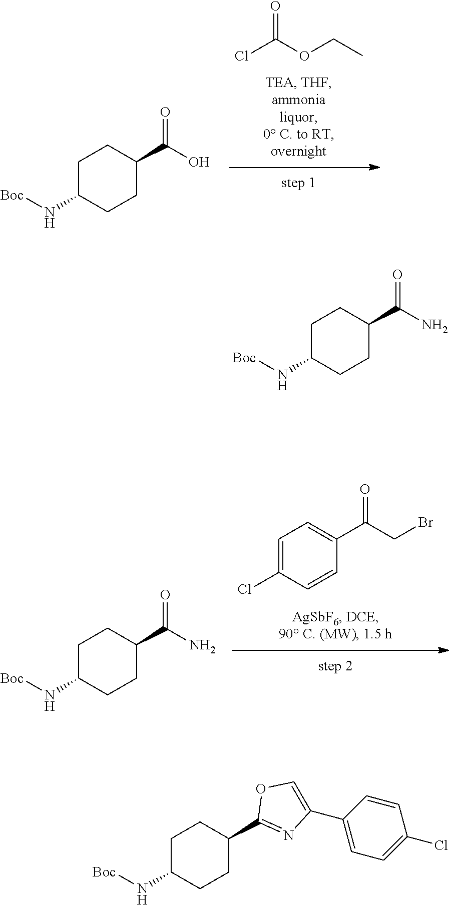

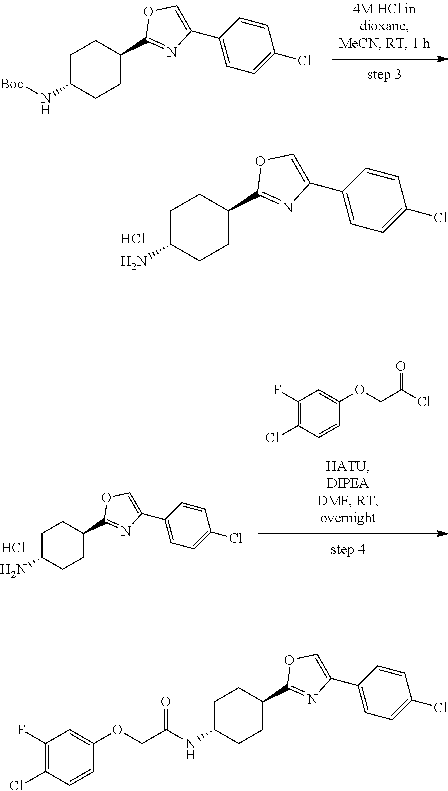

### Step 1: Synthesis of trans-tert-butyl (4-carbamoylcyclohexyl)carbamate

To a stirred solution of trans-4-((tert-butoxycarbonyl)amino)cyclohexane-1-carboxylic acid (500 mg, 2.05 mmol, 1.0 eq.) and ethyl chloroformate (445 mg, 4.11 mmol, 2.0 eq.) in THE (5 ml) was added TEA (1.0 mL, 6.16 mmol, 3.0 eq.) at 0° C., and the resulting reaction mixture was stirred at room temperature for 2 hours. Ammonia liquor (1.5 mL) was added dropwise and the resulting reaction mixture was stirred at room temperature overnight. The reaction was monitored by LCMS and TLC. Upon completion, the reaction mixture was poured into water (30 ml). The resulting solid was filtered off, washed with water, and dried under vacuum to yield trans-tert-butyl (4-carbamoylcyclohexyl)carbamate (409 mg, 82% yield) as a white solid. LCMS 243.1 [M+H]⁺.

### Step 2: Synthesis of trans-tert-butyl (4-(4-(4-chlorophenyl)oxazol-2-yl)cyclohexyl)carbamate

To a stirred solution of trans-tert-butyl (4-carbamoylcyclohexyl)carbamate (130 mg, 0.535 mmol, 1.0 eq.) in DCE (4 mL) was added 2-bromo-1-(4-chlorophenyl)ethan-1-one (149 mg, 0.643 mmol, 1.2 eq.) and AgSbF₆ (57 mg, 0.535 mmol, 1.0 eq.). The resulting reaction mixture was stirred in a microwave reactor at 90° C. for 1.5 hours. The reaction was monitored by LCMS. Upon completion, the reaction mixture was diluted with water (20 mL) and extracted with ethyl acetate (20 mL×2). The combined organic layers were washed with water (15 mL×2), dried over anhydrous Na₂SO₄, and concentrated under reduced pressure. The crude product was purified by flash chromatography (0-15% ethyl acetate in hexane as an eluent) to obtain trans-tert-butyl (4-(4-(4-chlorophenyl)oxazol-2-yl)cyclohexyl)carbamate (50 mg, 24% yield) as a white solid. LCMS 377.5 [M+H]⁺.

### Step 3: Synthesis of trans-4-(4-(4-chlorophenyl)oxazol-2-yl)cyclohexan-1-amine hydrochloride

To a stirred solution trans-tert-butyl (4-(4-(4-chlorophenyl)oxazol-2-yl)cyclohexyl)carbamate (40 mg, 0.106 mmol, 1.0 eq.) in MeCN (4 mL) was added 4 M HCl in dioxane (4 mL), and the resulting reaction mixture was stirred at RT for 1 hour. The reaction was monitored by LCMS. Upon completion, the reaction mixture was concentrated under reduced pressure. The crude product was crystallized in diethyl ether and dried under vacuum to obtain trans-4-(4-(4-chlorophenyl)oxazol-2-yl)cyclohexan-1-amine hydrochloride (40 mg) as a white solid. LCMS 277.3 [M+H]⁺.

### Step 4: Synthesis of trans-2-(4-chloro-3-fluorophenoxy)-N-(4-(4-(4-chlorophenyl)oxazol-2-yl)cyclohexyl)acetamide

To a stirred solution of trans-4-(4-(4-chlorophenyl)oxazol-2-yl)cyclohexan-1-amine hydrochloride (40 mg, 0.144 mmol, 1.0 eq.) and 2-(4-chloro-3-fluorophenoxy)acetic acid (44 mg, 0.217 mmol, 1.5 eq.) in DMF (2 mL) was added HATU (108 mg, 0.288 mmol, 2.0 eq.) and DIPEA (0.07 mL, 0.432 mmol, 3.0 eq.). The resulting reaction mixture was stirred at RT overnight. The reaction was monitored by LCMS. Upon completion, the reaction mixture was poured into water (10 ml) and extracted with ethyl acetate (20 mL×2). The combined organic layers were washed with water (10 mL), dried over anhydrous Na₂SO₄, and concentrated under reduced pressure. The crude product was purified by flash chromatography (0-40% ethyl acetate in hexane as an eluent) to obtain trans-2-(4-chloro-3-fluorophenoxy)-N-(4-(4-(4-chlorophenyl)oxazol-2-yl)cyclohexyl)acetamide (Compound 23-18 mg, 26% yield) as a white solid. LCMS 463.4 [M+H]⁺; ¹H NMR (500 MHz, DMSO-d6) δ 8.55 (s, 1H), 8.05 (d, J=7.9 Hz, 1H), 7.77 (d, J=7.8 Hz, 2H), 7.54-7.45 (m, 3H), 7.11-7.05 (m, 1H), 6.85 (d, J=9.1 Hz, 1H), 4.52 (s, 2H), 3.68 (dq, J=11.2, 7.2 Hz, 1H), 2.81 (dd, J=13.8, 10.2 Hz, 1H), 2.11 (t, J=12.2 Hz, 2H), 1.91-1.84 (m, 2H), 1.59 (q, J=13.1 Hz, 2H), 1.47-1.36 (m, 2H).

### Example 24

### Synthesis of trans-2-(4-chloro-3-fluorophenoxy)-N-(4-(5-(4,4-difluorocyclohexyl)-1,3,4-oxadiazol-2-yl)cyclohexyl)acetamide

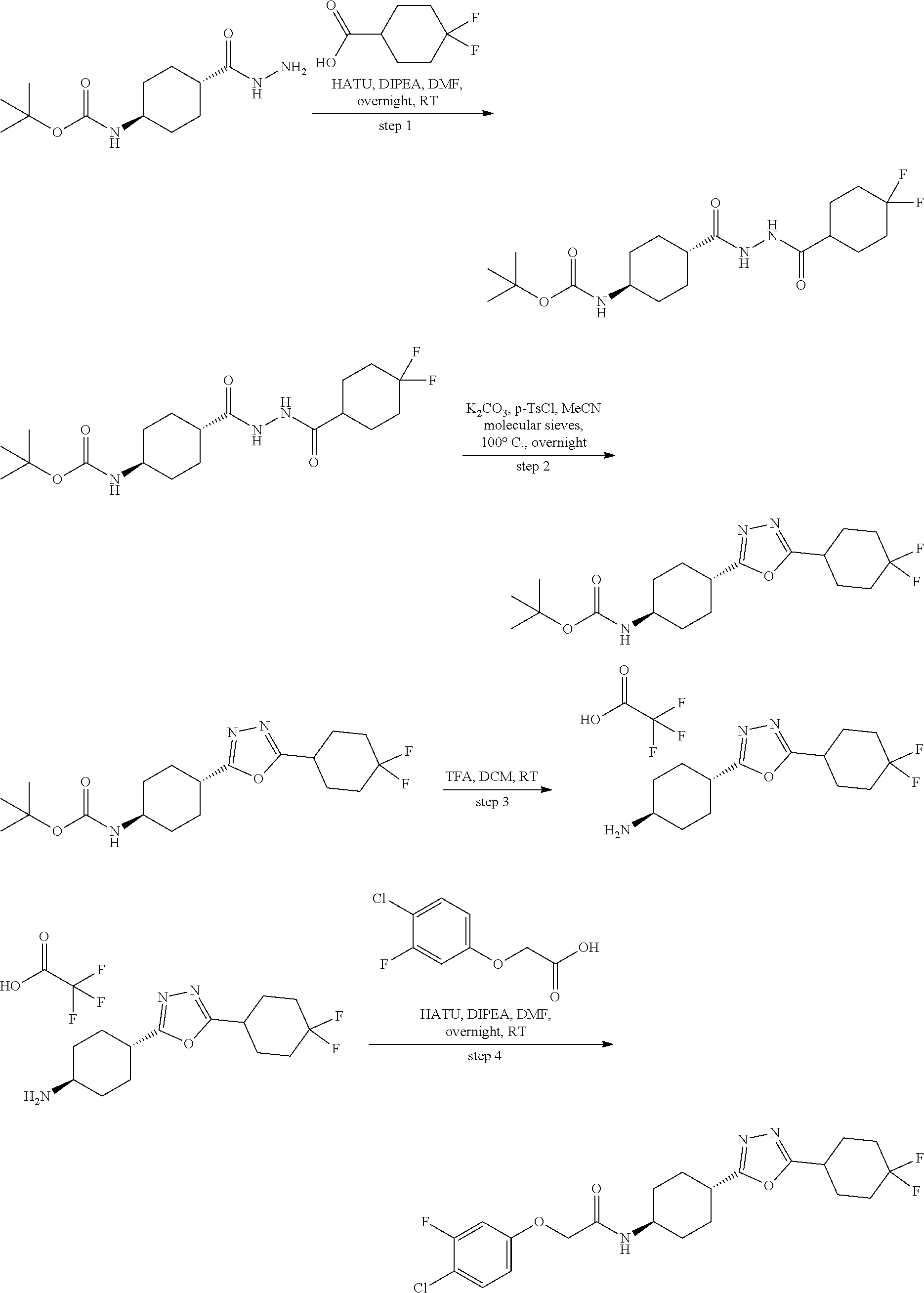

### Step 1: Synthesis of trans-tert-butyl 4-(2-(4,4-difluorocyclohexane-1-carbonyl)hydrazine-l-carbonyl)cyclohexyl)carbamate

To a stirred mixture of trans-tert-butyl 4-(hydrazinecarbonyl)cyclohexylcarbamate (500 mg, 1.945 mmol, 1 eq.), 4,4-difluorocyclohexanecarboxylic acid (383 mg, 2.335 mmol, 1.2 eq.), and HATU (1001 mg, 2.92 mmol, 1.5 eq.) in DMF (5 mL) was added DIPEA (1.4 mL, 7.78 mmol, 4.0 eq.), and the resulting reaction mixture was stirred at RT overnight. The product formation was confirmed by LCMS. Upon completion, the reaction mixture was poured into ice cold water (100 ml). The resulting solid was filtered off and dried under vacuum to obtain trans-tert-butyl 4-(2-(4,4-difluorocyclohexane-1-carbonyl)hydrazine-1-carbonyl)cyclohexyl)carbamate (690 mg, 88% yield) as an off white solid. LCMS 404.5 [M+H]⁺.

### Step 2: Synthesis of trans-tert-butyl (4-(5-(4,4-difluorocyclohexyl)-1,3,4-oxadiazol-2-yl)cyclohexyl)carbamate

To a stirred mixture of trans-tert-butyl 4-(2-(4,4-difluorocyclohexane-1-carbonyl)hydrazine-1-carbonyl)cyclohexyl)carbamate (600 mg, 1.488 mmol, 1 eq.), K₂CO₃ (1026 mg, 7.44 mmol, 5.0 eq.), and molecular sieves (1000 mg) in acetonitrile (20 mL) was added 4-toluenesulfonyl chloride (707 mg, 3.72 mmol, 2.5 eq.), and the resulting reaction mixture was stirred at 100° C. overnight. The product formation was confirmed by LCMS. Upon completion, the reaction mixture was filtered through Celite®. The filtrate was diluted with EtOAc (100 mL), washed with water (50 mL) and brine (50 mL), dried over anhydrous Na₂SO₄, and concentrated. The crude product was washed with pentane to obtain trans-tert-butyl (4-(5-(4,4-difluorocyclohexyl)-1,3,4-oxadiazol-2-yl)cyclohexyl)carbamate (515 mg, 89% yield) as an off white solid. LCMS 386.3 [M+H]⁺.

### Step 3: Synthesis of trans-4-(5-(4,4-difluorocyclohexyl)-1,3,4-oxadiazol-2-yl)cyclohexan-1-amine 2,2,2-trifluoroacetate

To a stirred solution of trans-tert-butyl (4-(5-(4,4-difluorocyclohexyl)-1,3,4-oxadiazol-2-yl)cyclohexyl)carbamate (600 mg, 1.56 mmol, 1 eq.) in DCM (10 mL) was added trifluoroacetic acid (0.5 mL), and the resulting reaction mixture was stirred at RT overnight under nitrogen atmosphere. The product formation was confirmed by LCMS. Upon completion, the reaction mixture was concentrated under reduced pressure. The crude product was crystallized in diethyl ether and dried under vacuum to obtain trans-4-(5-(4,4-difluorocyclohexyl)-1,3,4-oxadiazol-2-yl)cyclohexan-1-amine 2,2,2-trifluoroacetate (600 mg, 92% yield) as an off white solid. LCMS 286.3 [M+H]⁺.

### Step 4: Synthesis of trans-2-(4-chloro-3-fluorophenoxy)-N-(4-(5-(4,4-difluorocyclohexyl)-1,3,4-oxadiazol-2-yl)cyclohexyl)acetamide

To a stirred solution of trans-4-(5-(4,4-difluorocyclohexyl)-1,3,4-oxadiazol-2-yl)cyclohexan-1-amine 2,2,2-trifluoroacetate (200 mg, 0.501 mmol, 1 eq.), 2-(4-chloro-3-fluorophenoxy)acetic acid (102 mg, 0.501 mmol, 1 eq.), and HATU (285 mg, 0.752 mmol, 1.5 eq.) in DMF (4 mL) was added DIPEA (0.4 mL, 2.004 mmol, 4.0 eq.), and the resulting reaction mixture was stirred at RT overnight. The product formation was confirmed by LCMS. Upon completion, the reaction mixture was diluted with EtOAc (100 mL), washed with water (4×50 mL) and brine (50 mL), dried over anhydrous Na₂SO₄, and concentrated under vacuum. The crude product was purified by flash chromatography (0-2.5% MeOH in DCM as eluent) to obtain trans-2-(4-chloro-3-fluorophenoxy)-N-(4-(5-(4,4-difluorocyclohexyl)-1,3,4-oxadiazol-2-yl)cyclohexyl)acetamide (Compound 24-220 mg, 93% yield) as a white solid. LCMS 472.5 [M+H]⁺; ¹H NMR (400 MHz, DMSO-d6) δ 8.05 (d, J=7.9 Hz, 1H), 7.51 (t, J=8.9 Hz, 1H), 7.08 (dd, J=11.4, 2.8 Hz, 1H), 6.86 (ddd, J=9.0, 2.9, 1.2 Hz, 1H), 4.52 (s, 2H), 3.67 (dtd, J=11.6, 7.7, 4.0 Hz, 1H), 3.16 (dd, J=12.1, 8.5 Hz, 1H), 2.87 (tt, J=11.9, 3.6 Hz, 1H), 2.14-1.89 (m, 8H), 1.91-1.70 (m, 4H), 1.56 (qd, J=13.0, 3.1 Hz, 2H), 1.48-1.33 (m, 2H).

### Example 25

### Synthesis of trans-2-(4-chloro-3-fluorophenoxy)-N-(4-(5-(4-(trifluoromethyl)cyclohexyl)-1,3,4-oxadiazol-2-yl)cyclohexyl)acetamide

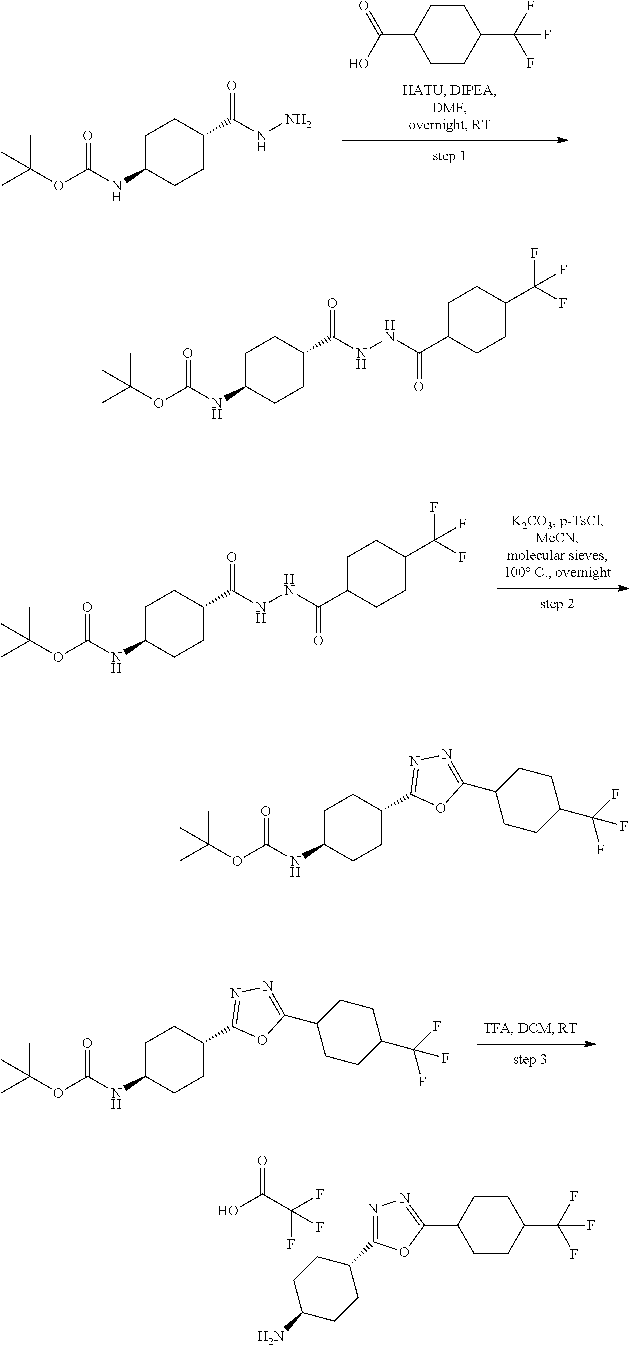

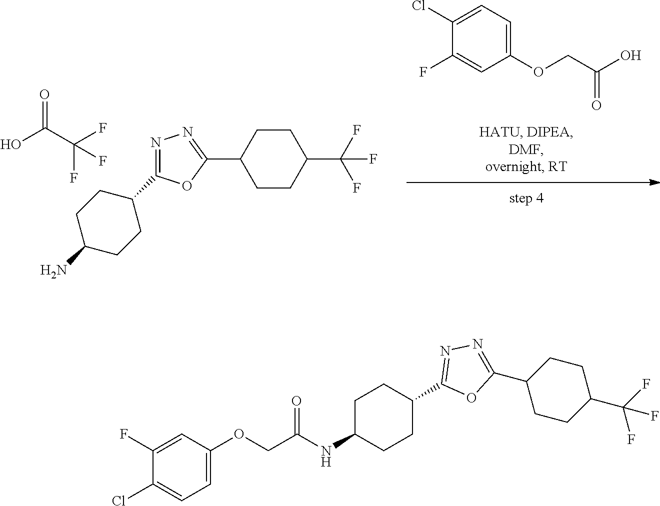

### Step 1: Synthesis of trans-tert-butyl (4-(2-(4-(trifluoromethyl)cyclohexane-1-carbonyl)hydrazine-1-carbonyl)cyclohexyl)carbamate

To a stirred mixture of tert-butyl 4-(hydrazinecarbonyl)cyclohexylcarbamate (200 mg, 0.77 mmol, 1 eq.), 4-(trifluoromethyl)cyclohexanecarboxylic acid (152 mg, 2.335 mmol, 1.2 eq.), and HATU (442 mg, 1.165 mmol, 1.5 eq.) in DMF (5 mL) was added DIPEA (0.5 mL, 3.108 mmol, 4.0 eq.), and the resulting reaction mixture was stirred at RT overnight. The product formation was confirmed by LCMS. Upon completion, the reaction mixture was poured into ice cold water (100 ml). The resulting solid was filtered off and dried under vacuum to obtain trans-tert-butyl (4-(2-(4-(trifluoromethyl)cyclohexane-1-carbonyl)hydrazine-1-carbonyl)cyclohexyl)carbamate (200 mg, 59% yield) as an off white solid. LCMS 436.6 [M+H]⁺.

### Step 2: Synthesis of trans-tert-butyl (4-(5-(4-(trifluoromethyl)cyclohexyl)-1,3,4-oxadiazol-2-yl)cyclohexyl)carbamate

To a stirred mixture of trans-tert-butyl (4-(2-(4-(trifluoromethyl)cyclohexane-1-carbonyl)hydrazine-1-carbonyl)cyclohexyl)carbamate (500 mg, 1.148 mmol, 1 eq.), K₂CO₃ (792 mg, 5.74 mmol, 5.0 eq.), and molecular sieves (1000 mg) in acetonitrile (15 mL) was added 4-toluenesulfonyl chloride (545 mg, 2.87 mmol, 2.5 eq.), and the resulting reaction mixture was stirred at 100° C. overnight. The product formation was confirmed by LCMS. Upon completion, the reaction mixture was filtered through Celite®, diluted with EtOAc (100 mL), washed with water (50 mL) and brine (50 mL), dried over anhydrous Na₂SO₄, and concentrated. The crude product was washed with pentane to obtain trans-tert-butyl (4-(5-(4-(trifluoromethyl)cyclohexyl)-1,3,4-oxadiazol-2-yl)cyclohexyl)carbamate (450 mg, 90% yield) as an off white solid. LCMS 418.4 [M+H]⁺.

### Step 3: Synthesis of trans-4-(5-(4-(trifluoromethyl)cyclohexyl)-1,3,4-oxadiazol-2-yl)cyclohexan-1-amine 2,2,2-trifluoroacetate

To a stirred solution of trans-tert-butyl (4-(5-(4-(trifluoromethyl)cyclohexyl)-1,3,4-oxadiazol-2-yl)cyclohexyl)carbamate (300 mg, 0.719 mmol, 1 eq.) in DCM (10 mL) was added trifluoroacetic acid (0.5 mL). The resulting reaction mixture was stirred at RT overnight under nitrogen atmosphere. The product formation was confirmed by LCMS. Upon completion, the reaction mixture was concentrated under reduced pressure. The crude product was crystallized in diethyl ether and dried under vacuum to obtain trans-4-(5-(4-(trifluoromethyl)cyclohexyl)-1,3,4-oxadiazol-2-yl)cyclohexan-1-amine 2,2,2-trifluoroacetate (300 mg, 96% yield) as an off white solid. LCMS 318.3 [M+H]⁺.

### Step 4: Synthesis of trans-2-(4-chloro-3-fluorophenoxy)-N-(4-(5-(4-(trifluoromethyl)cyclohexyl)-1,3,4-oxadiazol-2-yl)cyclohexyl)acetamide

To a stirred solution of trans-4-(5-(4-(trifluoromethyl)cyclohexyl)-1,3,4-oxadiazol-2-yl)cyclohexan-1-amine 2,2,2-trifluoroacetate (250 mg, 0.580 mmol, 1 eq.), 2-(4-chloro-3-fluorophenoxy)acetic acid (118 mg, 0.580 mmol, 1 eq.), and HATU (330 mg, 0.87 mmol, 1.5 eq.) in DMF (4 mL) was added DIPEA (0.4 mL, 2.32 mmol, 4 eq.). The resulting reaction mixture was stirred at RT overnight. The product formation was confirmed by LCMS. Upon completion, the reaction mixture was diluted with EtOAc (100 mL), washed with water (4×50 mL) and brine (50 mL), dried over anhydrous Na₂SO₄, and concentrated under vacuum. The crude product was purified by flash chromatography (0-2.5% MeOH in DCM as eluent) to obtain trans-2-(4-chloro-3-fluorophenoxy)-N-(4-(5-(4-(trifluoromethyl)cyclohexyl)-1,3,4-oxadiazol-2-yl)cyclohexyl)acetamide (Compound 25-200 mg, 69% yield) as a white solid. LCMS 504.5 [M+H]⁺; ¹H NMR (400 MHz, DMSO-d₆) δ 8.03 (d, J=7.9 Hz, 1H), 7.50 (t, J=8.9 Hz, 1H), 7.07 (dd, J=11.4, 2.9 Hz, 1H), 6.89-6.81 (m, 1H), 4.51 (s, 2H), 3.66 (s, 1H), 3.00-2.80 (m, 2H), 2.69 (s, 1H), 2.11 (t, J=14.1 Hz, 4H), 2.00-1.71 (m, 6H), 1.53 (dd, J=14.6, 11.1 Hz, 2H), 1.41 (d, J=14.3 Hz, 4H).

### Example 26

### Synthesis of trans-2-(4-chloro-3-fluorophenoxy)-N-(4-(4-(4-chlorophenyl)-1H-pyrazol-1-yl)cyclohexyl)acetamide

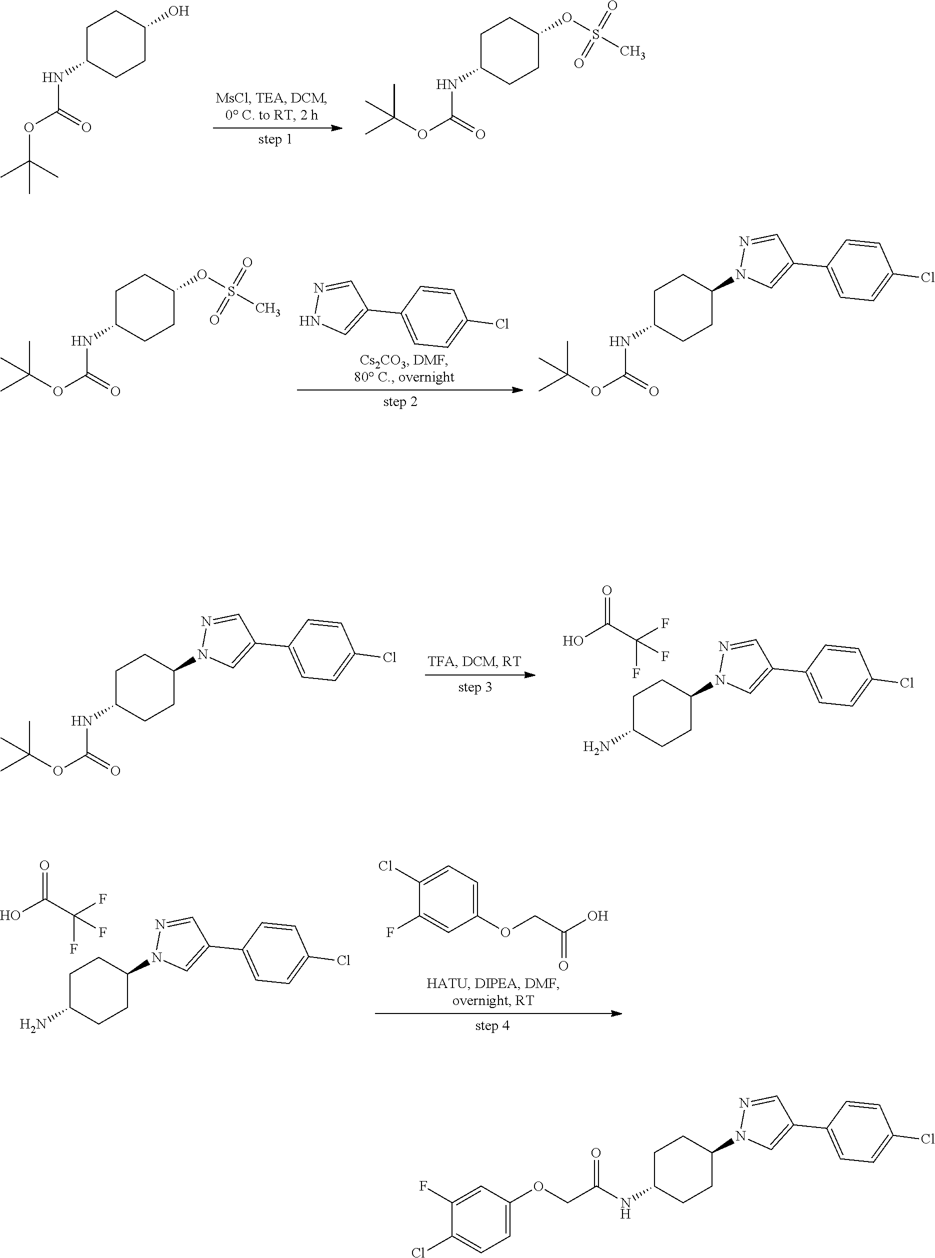

### Step 1: Synthesis of cis-4-((tert-butoxycarbonyl)amino)cyclohexyl methanesulfonate

To a stirred solution of cis-tert-butyl (4-hydroxycyclohexyl)carbamate (1000 mg, 4.65 mmol, 1 eq.) in DCM (10 mL) was added TEA (1.2 mL, 9.30 mmol, 2.0 eq.), followed by the addition of methanesulfonyl chloride (0.36 mL, 4.65 mmol, 1.0 eq.) at 0° C. The resulting reaction mixture was stirred at RT for 2 hours. The product formation was confirmed by NMR. Upon completion, the reaction mixture was diluted with water and extracted with DCM (100 mL). The DCM was then washed with water (20 mL), dried over anhydrous Na₂SO₄, and concentrated under reduced pressure to obtain cis-4-((tert-butoxycarbonyl)amino)cyclohexyl methanesulfonate (800 mg, 58% yield) as an off white solid. ¹H NMR (400 MHz, DMSO-d₆) δ 6.85 (d, J=7.5 Hz, 1H), 4.76 (br. s., 1H), 3.25-3.11 (m, 3H), 3.11-3.01 (m, 1H), 2.02-1.87 (m, 2H), 1.70-1.58 (m, 4H), 1.52-1.42 (m, 2H), 1.42-1.34 (m, 9H).

### Step 2: Synthesis of trans-tert-butyl (4-(4-(4-chlorophenyl)-1H-pyrazol-1-yl)cyclohexyl)carbamate

To a stirred solution of cis-4-((tert-butoxycarbonyl)amino)cyclohexyl methanesulfonate (395 mg, 1.34 mmol, 1.2 eq.) and 4-(4-chlorophenyl)-1H-pyrazole (200 mg, 1.12 mmol, 1 eq.) in DMF (5 mL) was added Cs₂CO₃ (728 mg, 2.24 mmol, 2.0 eq.) at RT. The resulting reaction mixture was heated at 80° C. overnight. The product formation was confirmed by LCMS. Upon completion, the reaction mixture was diluted with water, extracted with EtOAc (100 mL). Organic layer was washed with water (20 mL), dried over anhydrous Na₂SO₄, and concentrated under reduced pressure. The crude product was purified by flash chromatography (0-20% ethyl acetate in hexane as an eluent) to obtain trans-tert-butyl (4-(4-(4-chlorophenyl)-1H-pyrazol-1-yl)cyclohexyl)carbamate (30 mg, 8% yield) as an off white solid. LCMS 376.3 [M+H]⁺.

### Step 3: Synthesis of trans-4-(4-(4-chlorophenyl)-1H-pyrazol-1-yl)cyclohexan-1-amine 2,2,2-trifluoroacetate

To a stirred solution of trans-tert-butyl (4-(4-(4-chlorophenyl)-1H-pyrazol-1-yl)cyclohexyl)carbamate (30 mg, 0.08 mmol, 1 eq.) in DCM (2 mL) was added trifluoroacetic acid (0.2 mL), and the resulting reaction mixture was stirred at RT overnight. The product formation was confirmed by LCMS. Upon completion, the reaction mixture was concentrated under reduced pressure. The crude product was crystallized in diethyl ether and dried under vacuum to obtain trans-4-(4-(4-chlorophenyl)-1H-pyrazol-1-yl)cyclohexan-1-amine 2,2,2-trifluoroacetate (15 mg, 48.3% yield) as an off white solid. LCMS 276.2 [M+H]⁺.

### Step 4: Synthesis of trans-2-(4-chloro-3-fluorophenoxy)-N-(4-(4-(4-chlorophenyl)-1H-pyrazol-1-yl)cyclohexyl)acetamide

To a stirred solution of trans-4-(4-(4-chlorophenyl)-1H-pyrazol-1-yl)cyclohexan-1-amine 2,2,2-trifluoroacetate (20 mg, 0.051 mmol, 1 eq.), 2-(4-chloro-3-fluorophenoxy)acetic acid (12 mg, 0.061 mmol, 1.2 eq.), and HATU (38 mg, 0.102 mmol, 2.0 eq.) in DMF (0.5 mL) was added DIPEA (13 mg, 0.102 mmol, 2.0 eq.), and the resulting reaction mixture was stirred at RT overnight. The product formation was confirmed by LCMS. Upon completion, the reaction mixture was diluted with water and extracted with EtOAc (50 mL). The EtOAc was then washed with water (20 mL) & brine (20 mL), dried over anhydrous Na₂SO₄, and concentrated. The crude product was crystallized in methanol to obtain trans-2-(4-chloro-3-fluorophenoxy)-N-(4-(4-(4-chlorophenyl)-1H-pyrazol-1-yl)cyclohexyl)acetamide (Compound 26-7 mg, 30%) as an off white solid. LCMS 462.4 [M+H]⁺; ¹H NMR (400 MHz, DMSO-d₆) δ 8.28 (s, 1H), 8.07 (d, J=7.9 Hz, 1H), 7.89 (s, 1H), 7.68-7.56 (m, J=8.8 Hz, 2H), 7.51 (t, J=8.8 Hz, 1H), 7.45-7.32 (m, J=8.8 Hz, 2H), 7.08 (dd, J=3.1, 11.4 Hz, 1H), 6.86 (d, J=8.8 Hz, 1H), 4.52 (s, 2H), 4.14 (d, J=11.0 Hz, 1H), 3.72 (d, J=7.9 Hz, 1H), 2.09 (d, J=12.7 Hz, 2H), 1.93-1.64 (m, 4H), 1.57-1.42 (m, 2H).

### Example 27

### Synthesis of trans-4-(5-(4-chlorophenyl)-1,3,4-thiadiazol-2-yl)-N-(6-chloroquinolin-2-yl)cyclohexane-1-carboxamide

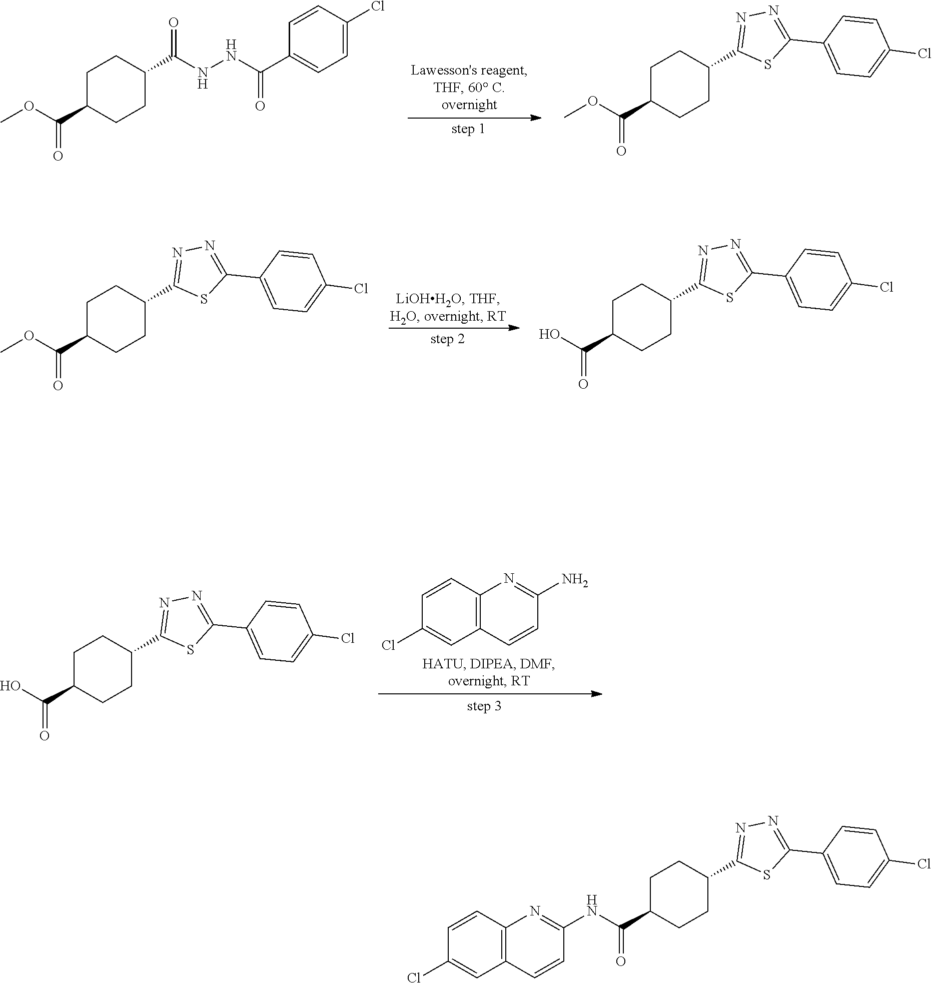

### Step 1: Synthesis of trans-methyl 4-(5-(4-chlorophenyl)-1,3,4-thiadiazol-2-yl)cyclohexane-1-carboxylate

To a stirred solution of trans-methyl 4-(2-(4-chlorobenzoyl)hydrazine-1-carbonyl)cyclohexane-1-carboxylate (200 mg, 0.591 mmol, 1 eq.) in THF (10 mL) was added Lawesson's reagent (358 mg, 0.887 mmol, 1.5 eq.) at RT. The resulting reaction mixture was heated at 100° C. overnight. The product formation was confirmed by LCMS and TLC. Upon completion, the reaction mixture was diluted with water and then extracted into DCM (100 mL). The DCM was then washed with NaHCO₃ (20 mL) & brine (20 mL), dried over anhydrous Na₂SO₄, and concentrated under reduced pressure. The crude product was purified by flash chromatography (0-30% ethyl acetate in hexane as an eluent) to obtain trans-methyl 4-(5-(4-chlorophenyl)-1,3,4-thiadiazol-2-yl)cyclohexane-1-carboxylate (120 mg, 60% yield) as an off white solid. LCMS 337.2 [M+H]⁺; ¹H NMR (400 MHz, DMSO-d₆) δ 8.03-7.89 (m, 2H), 7.67-7.58 (m, J=8.3 Hz, 2H), 3.62 (s, 3H), 3.22 (br. s., 1H), 2.17 (d, J=12.3 Hz, 2H), 2.06-1.89 (m, 3H), 1.67-1.45 (m, 4H).

### Step 2: Synthesis of trans-4-(5-(4-chlorophenyl)-1,3,4-thiadiazol-2-yl)cyclohexane-1-carboxylic acid

To a stirred solution of trans-methyl 4-(5-(4-chlorophenyl)-1,3,4-thiadiazol-2-yl)cyclohexane-1-carboxylate (120 mg, 0.357 mmol, 1.0 eq.) in THE (5 mL) was added LiOH.H₂O (18 mg, 0.428 mmol, 1.2 eq.) and H₂O (1 ml). The resulting reaction mixture was stirred at RT overnight. The product formation was confirmed by LCMS. Upon completion, the reaction mixture was diluted with water (25 mL) and acidified with 1 M HCl. The resulting solid was filtered off and dried under vacuum to obtain trans-4-(5-(4-chlorophenyl)-1,3,4-thiadiazol-2-yl)cyclohexane-1-carboxylic acid (80 mg, 70%) as a white solid. LCMS 323.2 [M+H]⁺; ¹H NMR (400 MHz, DMSO-d₆) δ 12.15 (br. s., 1H), 8.03-7.91 (m, J=8.3 Hz, 2H), 7.67-7.59 (m, J=8.8 Hz, 2H), 3.21 (d, J=11.4 Hz, 1H), 2.33 (br. s., 1H), 2.17 (d, J=9.6 Hz, 2H), 2.02 (d, J=10.1 Hz, 2H), 1.65-1.44 (m, 4H).

### Step 3: Synthesis of trans-4-(5-(4-chlorophenyl)-1,3,4-thiadiazol-2-yl)-N-(6-chloroquinolin-2-yl)cyclohexane-1-carboxamide

To a stirred solution of trans-4-(5-(4-chlorophenyl)-1,3,4-thiadiazol-2-yl)cyclohexane-1-carboxylic acid (80 mg, 0.248 mmol, 1 eq.), 6-chloroquinolin-2-amine (53 mg, 0.298 mmol, 1.2 eq.), and HATU (188 mg, 0.496 mmol, 2.0 eq.) in DMF (2 mL) was added DIPEA (63 mg, 0.496 mmol, 2.0 eq.), and the resultant reaction mixture was stirred at RT overnight. The product formation was confirmed by LCMS. Upon completion, the reaction mixture was diluted with water and extracted with EtOAc (50 mL). The EtOAc was then washed with water (20 mL) & brine (20 mL), dried over anhydrous Na₂SO₄, and concentrated. The crude product was purified by flash chromatography (0-5% MeOH in DCM as an eluent) followed by reverse phase HPLC to obtain trans-4-(5-(4-chlorophenyl)-1,3,4-thiadiazol-2-yl)-N-(6-chloroquinolin-2-yl)cyclohexane-1-carboxamide (Compound 27-9 mg, 7% yield) as a white solid. LCMS 483.4 [M+H]⁺; ¹H NMR (400 MHz, DMSO-d₆) δ 10.89 (s, 1H), 8.41-8.31 (m, 2H), 8.05 (d, J=2.2 Hz, 1H), 8.02-7.91 (m, J=8.3 Hz, 2H), 7.81 (d, J=9.2 Hz, 1H), 7.71 (dd, J=2.2, 9.2 Hz, 1H), 7.68-7.58 (m, J=8.8 Hz, 2H), 3.86 (br. s., 1H), 3.18 (s, 1H), 2.24 (d, J=10.1 Hz, 2H), 2.03 (d, J=11.8 Hz, 2H), 1.71-1.55 (m, 4H).

### Example 28

### Synthesis of trans-N-(6-chloroquinolin-2-yl)-4-(5-(4,4-difluorocyclohexyl)-1,3,4-oxadiazol-2-yl)cyclohexanecarboxamide

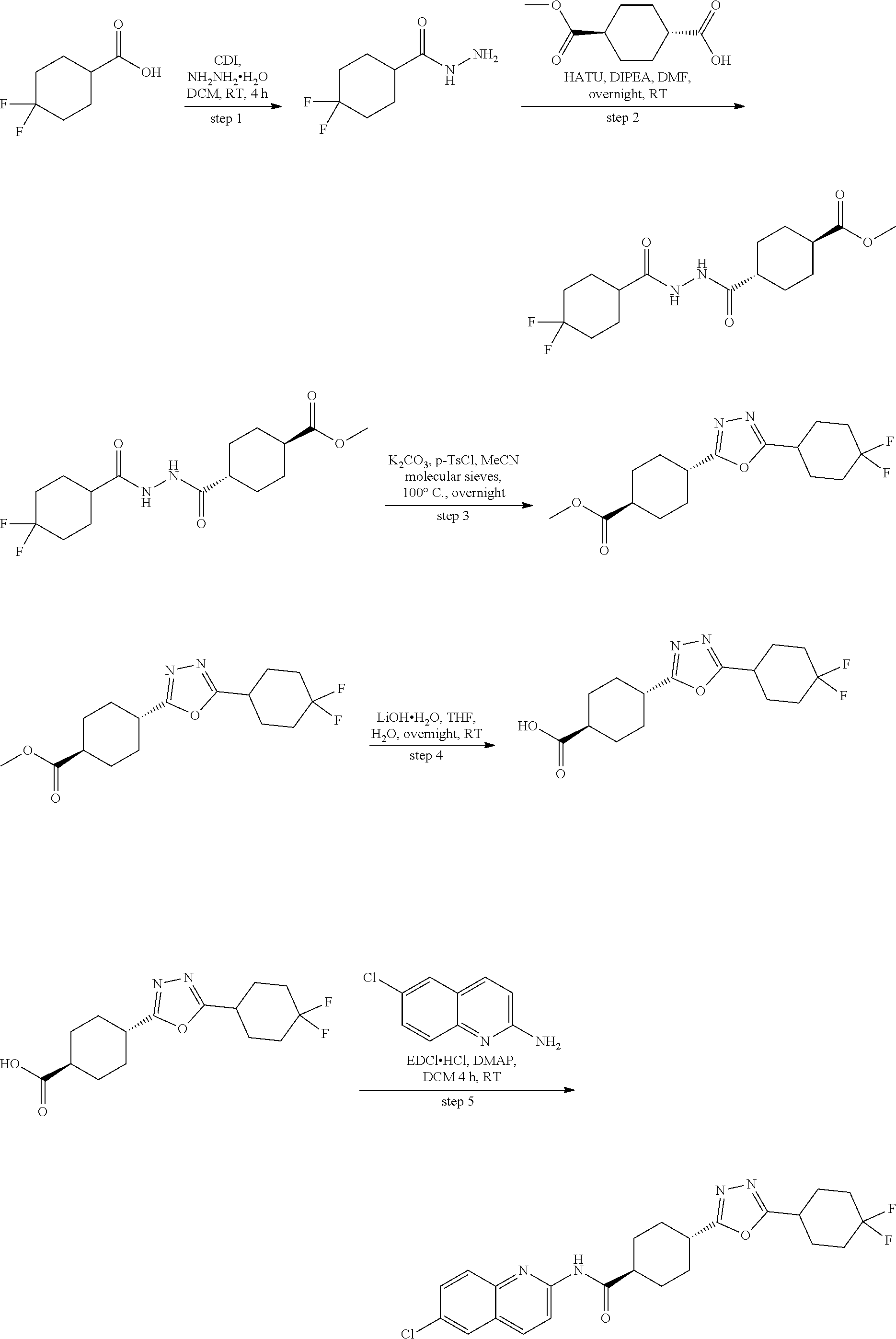

### Step 1: Synthesis of 4,4-difluorocyclohexanecarbohydrazide

To a stirred solution of 4,4-difluorocyclohexanecarboxylic acid (400 mg, 2.439 mmol, 1.0 eq.) in DCM (10 mL) was added Carbonyldiimidazole (593 mg, 3.658 mmol, 1.5 eq.). The resulting reaction mixture was allowed to stir at RT for 2 hours. Hydrazine hydrate (0.2 mL, 3.658 mmol, 1.5 eq.) was added to the reaction mixture and stirred at RT for 2 hours. Product formation was confirmed by LCMS. Upon completion, the reaction mixture was concentrated under reduced pressure. The resulting solid was washed with diethyl ether and dried under vacuum to obtain 4,4-difluorocyclohexanecarbohydrazide (440 mg, 100% yield) as a white solid. LCMS 179.2 [M+H]⁺.

### Step 2: Synthesis of trans-methyl 4-(2-(4,4-difluorocyclohexanecarbonyl)hydrazinecarbonyl)cyclohexanecarboxylate

To a stirred solution of 4,4-difluorocyclohexanecarbohydrazide (440 mg, 2.472 mmol, 1 eq.), trans-4-(methoxycarbonyl)cyclohexanecarboxylic acid (690 mg, 3.707 mmol, 1.2 eq.), and HATU (1400 mg, 3.708 mmol, 1.5 eq.) in DMF (10 mL) was added DIPEA (1.8 mL, 9.88 mmol, 4.0 eq.), and the resulting reaction mixture was stirred at RT overnight. Product formation was confirmed by LCMS. Upon completion of reaction, the reaction mixture was poured into ice cold water (200 ml). The resulting solid was filtered off and dried under vacuum to obtain trans-methyl 4-(2-(4,4-difluorocyclohexanecarbonyl)hydrazinecarbonyl)cyclohexanecarboxylate (700 mg, 82% yield) as white solid. LCMS 347.3 [M+H]⁺.

### Step 3: Synthesis of trans-methyl 4-(5-(4,4-difluorocyclohexyl)-1,3,4-oxadiazol-2-yl)cyclohexanecarboxylate

To a stirred solution of trans-methyl 4-(2-(4,4-difluorocyclohexanecarbonyl)hydrazinecarbonyl)cyclohexanecarboxylate (700 mg, 2.023 mmol, 1 eq.), K₂CO₃ (1400 mg, 10.12 mmol, 5.0 eq.) and molecular sieves (1000 mg) in acetonitrile (100 mL) was added 4-toluenesulfonyl chloride (960 mg, 5.057 mmol, 2.5 eq.) and the resulting reaction mixture was stirred at 100° C. overnight. Product formation was confirmed by LCMS. After completion of reaction, the reaction mixture was filtered and the filtrate was diluted with ethyl acetate (100 mL). Organic layer was washed with water (100 mL) and brine (100 mL), dried over anhydrous Na₂SO₄ and concentrated under reduced pressure to obtain methyl trans-4-(5-(4,4-difluorocyclohexyl)-1,3,4-oxadiazol-2-yl)cyclohexanecarboxylate (560 mg, 84% yield) as an off white solid. LCMS 329.5 [M+H]⁺.

### Step 4: Synthesis of trans-4-(5-(4,4-difluorocyclohexyl)-1,3,4-oxadiazol-2-yl)cyclohexanecarboxylic acid

To a stirred solution of trans-4-(5-(4,4-difluorocyclohexyl)-1,3,4-oxadiazol-2-yl)cyclohexanecarboxylate (500 mg, 1.52 mmol, 1 eq.) in THE (10 mL) and H₂O (10 mL) was added LiOH.H₂O (128 mg, 3.048 mmol, 1 eq.) and the resulting reaction mixture was stirred at RT overnight under nitrogen atmosphere. Product formation was confirmed by LCMS. After completion of reaction, the reaction mixture was concentrated under reduced pressure. The resulting residue was dissolved in water (20 mL) and extracted with ethyl acetate (20 mL). Aqueous layer was acidified with 6 m HCl (pH˜4 to 5). The resulting solid formed was filtered off and dried under vacuum to obtain trans-4-(5-(4,4-difluorocyclohexyl)-1,3,4-oxadiazol-2-yl)cyclohexanecarboxylic acid (400 mg, 83% yield) as an off white solid. LCMS 315.3 [M+H]⁺.

### Step 5: Synthesis of trans-N-(6-chloroquinolin-2-yl)-4-(5-(4,4-difluorocyclohexyl)-1,3,4-oxadiazol-2-yl)cyclohexanecarboxamide

To a stirred solution of trans-4-(5-(4,4-difluorocyclohexyl)-1,3,4-oxadiazol-2-yl)cyclohexanecarboxylic acid (60 mg, 0.19 mmol, 1 eq.) in DCM (20 mL) was added EDCI.HCl (54 mg, 0.285 mmol, 1.5 eq.) and DMAP (35 mg, 0.285 mmol, 1.5 eq.) at RT and stirred for 15 min. 6-chloroquinolin-2-amine (34 mg, 0.19 mmol, 1.0 eq.) was added and the resulting reaction mixture was stirred at RT for 4 h. Product formation was confirmed by LCMS and TLC. After completion of reaction, the reaction mixture was concentrated under reduced pressure and the residue was diluted with EtOAc (20 mL). Organic layer was washed with water (10 mL) and brine (10 mL), dried over anhydrous Na₂SO₄ and concentrated under reduced pressure. The crude product was purified by reversed phase HPLC to obtain trans-N-(6-chloroquinolin-2-yl)-4-(5-(4,4-difluorocyclohexyl)-1,3,4-oxadiazol-2-yl)cyclohexanecarboxamide (Compound 28-51 mg, 56.6% yield) as a white solid. LCMS 474.5 [M+H]⁺; ¹H NMR (400 MHz, DMSO-d₆) δ 10.88 (br. s., 1H), 8.44-8.28 (m, 2H), 8.05 (d, J=1.8 Hz, 1H), 7.86-7.76 (m, 1H), 7.74-7.63 (m, 1H), 3.17 (br. s., 1H), 2.93 (d, J=11.6 Hz, 1H), 2.62 (br. s., 1H), 2.20-2.08 (m, 4H), 2.08 (br. s., 2H), 1.98 (d, J=10.3 Hz, 4H), 1.77 (d, J=10.1 Hz, 2H), 1.69-1.40 (m, 4H).

### Example 29

### Synthesis of trans-N-(6-chloroquinolin-2-yl)-4-(5-(4-(trifluoromethyl)cyclohexyl)-1,3,4-oxadiazol-2-yl)cyclohexanecarboxamide

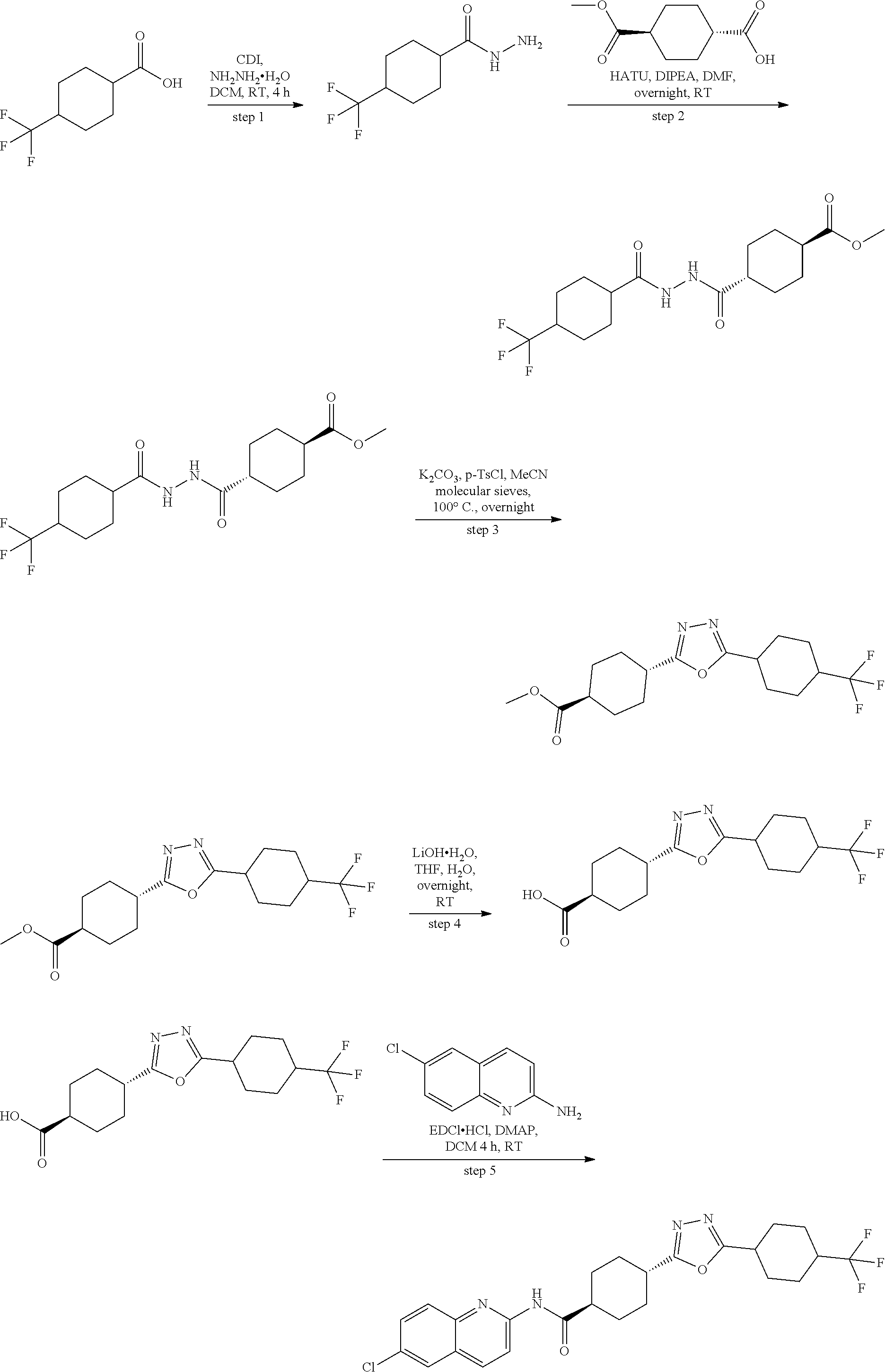

### Step 1: Synthesis of 4-(trifluoromethyl)cyclohexanecarbohydrazide

To a stirred solution of 4,4-difluorocyclohexanecarboxylic acid (500 mg, 2.549 mmol, eq.) in THF (5 mL) was added Carbonyldiimidazole (619 mg, 3.82 mmol, 1.5 eq.). The resulting reaction mixture was allowed to stir at RT for 2 hours. Hydrazine hydrate (0.2 mL, 3.82 mmol, 1.5 eq.) was added to reaction mixture and again stirred at RT for 2 hours. Product formation was confirmed by TLC. After completion of reaction, the reaction mixture was concentrated under reduced pressure to obtain 4-(trifluoromethyl)cyclohexanecarbohydrazide (520 mg, 97% yield) as a white solid (directly used for next step).

### Step 2: Synthesis of trans-methyl 4-(2-(4-(trifluoromethyl)cyclohexanecarbonyl)hydrazinecarbonyl)cyclohexanecarboxylate

To a stirred mixture of 4-(trifluoromethyl)cyclohexanecarbohydrazide (520 mg, 2.476 mmol, 1 eq.), trans-4-(methoxycarbonyl)cyclohexanecarboxylic acid (460 mg, 2.476 mmol, 1.0 eq.) and HATU (1411 mg, 3.71 mmol, 1.5 eq.) in DMF (4 mL) was added DIPEA (1.7 mL, 9.9 mmol, 4.0 eq.) and the resulting reaction mixture was stirred at RT overnight. Product formation was confirmed by LCMS. After completion of reaction, the reaction mixture was poured into ice cold water (200 ml). The resulting off white solid was filtered off and dried under vacuum to obtain trans-methyl 4-(2-(4-(trifluoromethyl)cyclohexanecarbonyl)hydrazinecarbonyl)cyclohexanecarboxylate (760 mg, 91% yield) as a white solid. LCMS 379.3 [M+H]⁺.

### Step 3: Synthesis of trans-methyl 4-(5-(4-(trifluoromethyl)cyclohexyl)-1,3,4-oxadiazol-2-yl)cyclohexanecarboxylate

To a stirred solution of trans-methyl 4-(2-(4-(trifluoromethyl)cyclohexanecarbonyl)hydrazinecarbonyl)cyclohexanecarboxylate (760 mg, 2.010 mmol, 1 eq.), K₂CO₃ (1400 mg, 10.05 mmol, 5.0 eq.) and molecular sieves (1000 mg) in acetonitrile (50 mL) was added 4-Toluenesulfonyl chloride (954 mg, 5.025 mmol, 2.5 eq.) and the resulting reaction mixture was stirred at 100° C. overnight. Product formation was confirmed by LCMS. After completion of reaction, the reaction mixture was filtered and the filtrate was diluted with ethyl acetate (100 mL). Organic layer was washed with water (100 mL) and brine (100 mL), dried over anhydrous Na₂SO₄ and concentrated under reduced pressure to obtain trans-methyl 4-(5-(4-(trifluoromethyl)cyclohexyl)-1,3,4-oxadiazol-2-yl)cyclohexanecarboxylate (700 mg, 96.8% yield) as off white solid. LCMS 361.3 [M+H]⁺.

### Step 4: Synthesis of trans-4-(5-(4-(trifluoromethyl)cyclohexyl)-1,3,4-oxadiazol-2-yl)cyclohexanecarboxylic acid

To a stirred solution of trans-methyl 4-(5-(4-(trifluoromethyl)cyclohexyl)-1,3,4-oxadiazol-2-yl)cyclohexanecarboxylate (700 mg, 1.94 mmol, 1 eq.) in THF (10 mL) and H₂O (10 mL) was added LiOH.H₂O (163 mg, 3.88 mmol, 2.0 eq.) and the resulting reaction mixture was stirred at RT overnight under nitrogen atmosphere. Product formation was confirmed by LCMS. After completion of reaction, the reaction mixture was concentrated under reduced pressure. The resulting residue was dissolved in water (20 mL) and extracted with ethyl acetate (20 mL). Aqueous layer was acidified with 6 M HCl (pH˜4 to 5). The resulting solid was filtered off and dried under vacuum to obtain trans-4-(5-(4-(trifluoromethyl)cyclohexyl)-1,3,4-oxadiazol-2-yl)cyclohexanecarboxylic acid (500 mg, 74% yield) as an off white solid. LCMS 347.3 [M+H]⁺.

### Step 5: Synthesis of trans-N-(6-chloroquinolin-2-yl)-4-(5-(4-(trifluoromethyl)cyclohexyl)-1,3,4-oxadiazol-2-yl)cyclohexanecarboxamide

To a stirred solution of trans-4-(5-(4-(trifluoromethyl)cyclohexyl)-1,3,4-oxadiazol-2-yl)cyclohexanecarboxylic acid (100 mg, 0.289 mmol, 1 eq.) in DCM (10 mL) was added EDCI.HCl (222 mg, 1.156 mmol, 4.0 eq.) and DMAP (99 mg, 0.809 mmol, 2.8 eq.). The resulting reaction mixture was stirred at RT for 5 minutes and followed by addition of 6-chloroquinolin-2-amine (48 mg, 0.268 mmol, 0.93 eq.). The reaction mixture was allowed to stir at RT for 2 hours. Product formation was confirmed by LCMS and TLC. After completion of reaction, the reaction mixture was concentrated under reduced pressure and the residue was diluted with EtOAc (20 mL). Organic layer was washed with water (10 mL) and brine (10 mL), dried over anhydrous Na₂SO₄ and concentrated under reduced pressure. The crude product was purified by flash chromatography (0-30% ethyl acetate in hexane as an eluent) to obtain trans-N-(6-chloroquinolin-2-yl)-4-(5-(4-(trifluoromethyl)cyclohexyl)-1,3,4-oxadiazol-2-yl)cyclohexanecarboxamide (Compound 29-80 mg, 27% yield) as a white solid. LCMS 507.5 [M+H]⁺; ¹H NMR (400 MHz, DMSO-d₆) δ 10.88 (s, 1H), 8.42-8.27 (m, 2H), 8.05 (d, J=2.4 Hz, 1H), 7.82 (s, 1H), 7.72 (dd, J=2.4, 9.0 Hz, 1H), 3.00-2.87 (m, 2H), 2.74-2.64 (m, 1H), 2.61 (d, J=9.2 Hz, 1H), 2.25 (d, J=7.5 Hz, 1H), 2.13 (br. s., 2H), 2.06 (d, J=6.6 Hz, 1H), 1.98 (d, J=7.2 Hz, 2H), 1.82-1.70 (m, 2H), 1.65-1.35 (m, 8H).

### Example 30

### Chiral separation of trans-N-(6-chloroquinolin-2-yl)-4-(5-(trans-4-(trifluoromethyl)cyclohexyl)-1,3,4-oxadiazol-2-yl)cyclohexanecarboxamide and trans-N-(6-chloroquinolin-2-yl)-4-(5-(cis-4-(trifluoromethyl)cyclohexyl)-1,3,4-oxadiazol-2-yl)cyclohexanecarboxamide

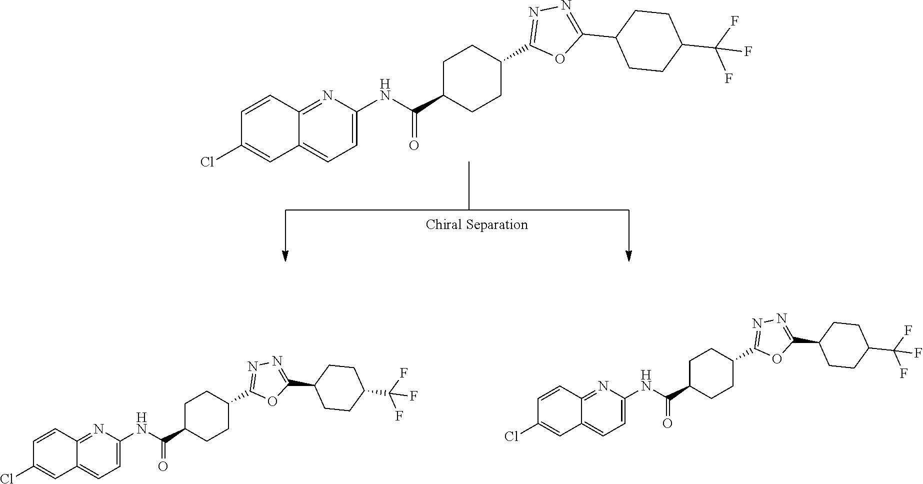

The enantiomers trans-N-(6-chloroquinolin-2-yl)-4-(5-(trans-4-(trifluoromethyl)cyclohexyl)-1,3,4-oxadiazol-2-yl)cyclohexanecarboxamide (Compound 30—[α]_{D}²⁰=NA; elution time: 18.63 min) and trans-N-(6-chloroquinolin-2-yl)-4-(5-(cis-4-(trifluoromethyl)cyclohexyl)-1,3,4-oxadiazol-2-yl)cyclohexanecarboxamide (Compound 31 [α]_{D}²⁰=NA; elution time: 25.42 min) were separated by chiral SFC (Chiralpak-IC, 250×21 mm, 5μ), using an isocratic program with analytical grade liquid carbon dioxide, HPLC grade MeOH, and HPLC grade Hexane (0.2% DEA). LCMS 507.5 [M+H]⁺.

### Example 31

### Synthesis of trans-4-(4-(4-chlorophenyl)oxazol-2-yl)-N-(6-chloroquinolin-2-yl)cyclohexanecarboxamide

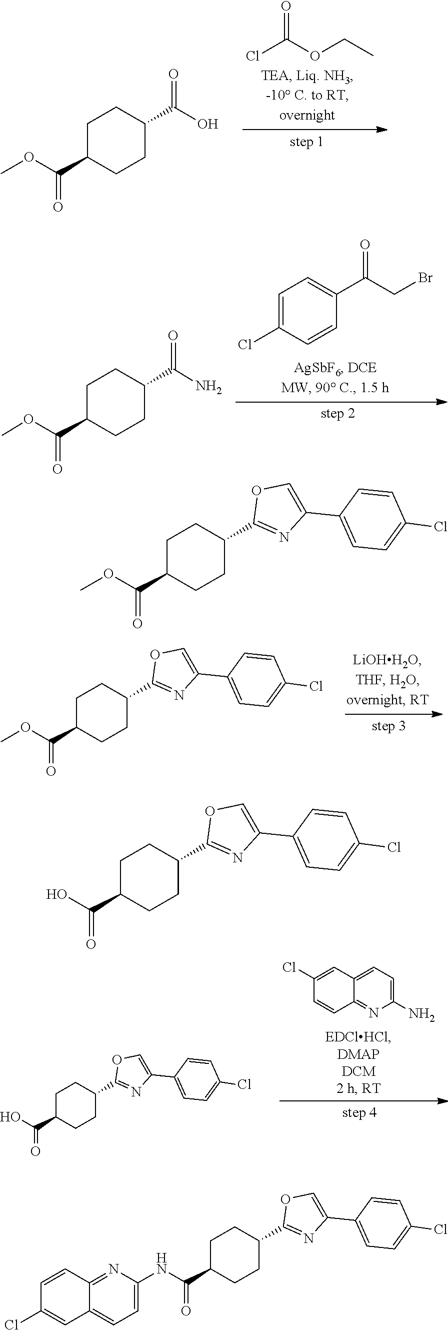

### Step 1: Synthesis of trans-methyl 4-carbamoylcyclohexanecarboxylate

To a stirred solution of trans-4-(methoxycarbonyl)cyclohexanecarboxylic acid (500 mg, 2.68 mmol, 1.0 eq.) in THF (10 mL) was added TEA (679 mg, 6.71 mmol, 2.5 eq.) and ethyl chloroformate (436 mg, 4.02 mmol, 1.5 eq.) at −10° C. under N₂ atmosphere. The resulting reaction mixture was stirred at RT for 3 hours. Liquid ammonia (0.5 mL) was added to the reaction mixture at −10° C., which continued stir at RT overnight. The reaction was monitored by NMR. After completion of reaction, the reaction mixture diluted with ethyl acetate (20 mL). The organic layer was separated and washed with water (10 mL) and brine (10 mL), dried over anhydrous Na₂SO₄ and concentrated under reduced pressure to obtain trans-methyl 4-carbamoylcyclohexanecarboxylate (378 mg, 76% yield) as a white solid. ¹H NMR (400 MHz, DMSO-d₆) δ 7.18 (br. s., 1H), 6.67 (br. s., 1H), 3.58 (s, 3H), 2.30-2.21 (m, 1H), 2.09-2.01 (m, 1H), 1.91 (d, J=10.1 Hz, 2H), 1.84-1.67 (m, 2H), 1.44-1.20 (m, 4H).

### Step 2: Synthesis of trans-methyl 4-(4-(4-chlorophenyl)oxazol-2-yl)cyclohexanecarboxylate

To a stirred solution of trans-methyl 4-carbamoylcyclohexanecarboxylate (200 mg, 1.08 mmol, 1.0 eq.) in DCE (10 mL) was added AgSbF₆ (111 mg, 0.324 mmol, 0.3 eq.) and 2-bromo-1-(4-chlorophenyl)ethanone (302 mg, 1.297 mmol, 1.2 eq.). The resulting reaction mixture was refluxed at 90° C. in microwave for 1.5 hours. Product formation was confirmed by LCMS and TLC. After completion of reaction, the reaction mixture was diluted with ethyl acetate (20 mL). The organic layer washed with water (10 mL) and brine (10 mL), dried over anhydrous Na₂SO₄ and concentrated under reduced pressure. The crude product was purified by flash chromatography (0-5% ethyl acetate in hexane as an eluent) to obtain trans-methyl 4-(4-(4-chlorophenyl)oxazol-2-yl)cyclohexanecarboxylate (100 mg, 29% yield) as an off white solid. LCMS 320.2 [M+H]⁺.

### Step 3: Synthesis of trans-4-(4-(4-chlorophenyl)oxazol-2-yl)cyclohexanecarboxylic acid

To a stirred solution of trans-methyl 4-(4-(4-chlorophenyl)oxazol-2-yl)cyclohexanecarboxylate (100 mg, 0.3134 mmol, 1.0 eq.) in THE (4 mL) and H₂O (4 mL) was added LiOH.H₂O (26 mg, 0.626 mmol, 2.0 eq.) and the resulting reaction mixture was stirred at RT overnight under nitrogen atmosphere. Product formation was confirmed by LCMS. After completion of reaction, the reaction mixture was concentrated under reduced pressure. The residue was dissolved in H₂O (5 mL) and washed with ethyl acetate (10 mL). The aqueous layer was acidified with 6 M HCl (pH˜4 to 5). The resulting solid was filtered off and dried under vacuum to obtain trans-4-(4-(4-chlorophenyl)oxazol-2-yl)cyclohexanecarboxylic acid (70 mg, 73% yield) as an off white solid. LCMS 306.3 [M+H]⁺.

### Step 4: Synthesis of trans-4-(4-(4-chlorophenyl)oxazol-2-yl)-N-(6-chloroquinolin-2-yl)cyclohexanecarboxamide

To a stirred solution of trans-4-(4-(4-chlorophenyl)oxazol-2-yl)cyclohexanecarboxylic acid (50 mg, 0.164 mmol, 1.0 eq.) in DCM (10 mL) was added EDCI.HCl (47 mg, 0.246 mmol, 1.5 eq.) and DMAP (30 mg, 0.246 mmol, 1.5 eq.). The resulting reaction mixture was stirred at RT for 5 minutes followed by the addition of 6-chloroquinolin-2-amine (29 mg, 0.1639 mmol, 1.0 eq.). The reaction mixture was stirred at RT for 2 hours. Product formation was confirmed by LCMS and TLC. After completion of reaction, the reaction mixture was concentrated under reduced pressure and the residue was diluted with EtOAc (20 mL). The organic layer was washed with water (10 mL) and brine (10 mL), dried over anhydrous Na₂SO₄ and concentrated under reduced pressure. The crude product was purified by flash chromatography (0-10% ethyl acetate in hexane as an eluent) to obtain trans-4-(4-(4-chlorophenyl)oxazol-2-yl)-N-(6-chloroquinolin-2-yl)cyclohexanecarboxamide (Compound 32-10 mg, 13% yield) as a yellow solid. LCMS 466.4 [M+H]⁺; ¹H NMR (400 MHz, DMSO-d₆) δ 10.86 (s, 1H), 8.56 (s, 1H), 8.40-8.27 (m, 2H), 8.05 (d, J=2.4 Hz, 1H), 7.88-7.77 (m, 3H), 7.71 (dd, J=2.4, 9.0 Hz, 1H), 7.49 (d, J=8.3 Hz, 2H), 2.90 (br. s., 1H), 2.67 (br. s., 2H), 2.17 (d, J=10.5 Hz, 2H), 2.00 (d, J=9.3 Hz, 2H), 1.69-1.40 (m, 4H).

### Example 32

### Synthesis of trans-4-(4-(4-chlorophenyl)thiazol-2-yl)-N-(6-chloroquinolin-2-yl)cyclohexanecarboxamide

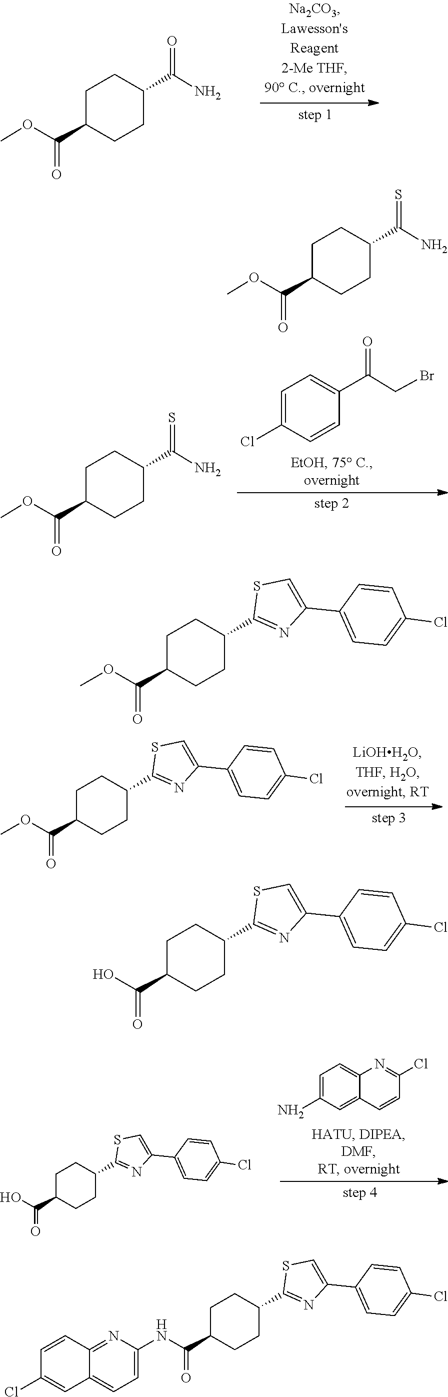

### Step 1: Synthesis trans-methyl 4-carbamothioylcyclohexanecarboxylate

To a solution of trans-methyl 4-carbamoylcyclohexanecarboxylate (0.200 g, 1.08 mmol, 1.0 eq.) in 2-methyl-THF (4 ml) was added Lawesson's reagent (0.521 g, 1.29 mmol, 1.2 eq.) followed by the addition of Na₂CO₃ (0.113 g, 1.08 mmol, 1.0 eq.). The resulting reaction mixture was heated at 90° C. overnight. Product formation was confirmed by LCMS and TLC. After completion of reaction, the reaction mixture was quenched with saturated sodium bicarbonate solution and extracted with ethyl acetate (50 mL×2). The combined organic layers were washed with water (20 mL×3), dried over anhydrous Na₂SO₄ and concentrated under reduced pressure to obtain trans-methyl 4-carbamothioylcyclohexanecarboxylate (0.150 g, 69% yield) as an off white solid. LCMS 202.1 [M+H]⁺.

### Step 2: Synthesis of trans-methyl 4-(4-(4-chlorophenyl)thiazol-2-yl)cyclohexanecarboxylate

A solution of trans-methyl 4-carbamothioylcyclohexanecarboxylate (0.100 g, 0.49 mmol, 1.0 eq.) and 2-bromo-1-(4-chlorophenyl)ethanone (0.114 g, 0.49 mmol, 1.0 eq.) in ethanol (5 ml) was heated at 75° C. overnight. Product formation was confirmed by LCMS and TLC. After completion of reaction, the reaction mixture was concentrated. The crude product was purified by flash chromatography (20-30% Ethyl acetate in hexane as an eluent) to obtain trans-methyl 4-(4-(4-chlorophenyl)thiazol-2-yl)cyclohexanecarboxylate (0.050 g, 30% yield) as a white solid. LCMS 336.2 [M+H]⁺.

### Step 3: Synthesis of trans-4-(4-(4-chlorophenyl)thiazol-2-yl)cyclohexanecarboxylic acid

To a stirred solution of trans-methyl 4-(4-(4-chlorophenyl)thiazol-2-yl)cyclohexanecarboxylate (0.100 g, 0.29 mmol, 1.0 eq.) in THE (2 mL) was added LiOH.H₂O (0.013 g, 0.58 mmol, 2.0 eq.) and H₂O (2 ml) and the resulting reaction mixture was stirred at RT for 2 h. Product formation was confirmed by LCMS. After completion of reaction, the reaction mixture was concentrated under reduced pressure. The residue was dissolved in water (10 mL) and acidified with 1 M HCl. The resulting solid was filtered off and dried under vacuum to obtain trans-4-(4-(4-chlorophenyl)thiazol-2-yl)cyclohexanecarboxylic acid (0.080 g, 84% yield) as an off white solid which was directly used in the next step.

### Step 4: Synthesis of trans-4-(4-(4-chlorophenyl)thiazol-2-yl)-N-(6-chloroquinolin-2-yl)cyclohexanecarboxamide

To a solution of trans-4-(4-(4-chlorophenyl)thiazol-2-yl)cyclohexanecarboxylic acid (0.025 g, 0.077 mmol, 1 eq.) in DMF (1.0 mL) was added 6-chloroquinolin-2-amine (0.016 g, 0.093 mmol, 1.2 eq.) and HATU (0.044 g, 0.116 mmol, 1.5 eq.) followed by the addition of DIPEA (0.04 mL, 0.116 mmol, 3.0 eq.) at room temperature. The resulting reaction mixture was stirred at RT overnight. Product formation was confirmed by LCMS. After completion of reaction, the reaction mixture was poured into water (2 ml) extracted with ethyl acetate (3 mL×3). The combined organic layers were washed with water (3 mL×2), dried over anhydrous Na₂SO₄ and concentrated under reduced pressure. The crude product was purified by flash chromatography (5-15% Ethyl acetate in hexane as an eluent) to obtain trans-4-(4-(4-chlorophenyl)thiazol-2-yl)-N-(6-chloroquinolin-2-yl)cyclohexanecarboxamide (Compound 33-0.005 g, 13% yield) as a white solid. LCMS 482.4 [M+H]⁺; ¹H NMR (400 MHz, DMSO-d₆) δ 10.87 (s, 1H) 8.32-8.38 (q, 2H) 8.05 (s, 2H) 8.02 (d, J=8.77 Hz, 2H) 7.81 (d, J=8.77 Hz, 1H) 7.56-7.78 (m, 2H) 3.07 (br. s., 1H) 2.67 (br. s., 1H) 2.25 (br. s., 2H) 2.02 (br. s., 2H) 1.52-1.71 (m, 4H).

### Example 33

### Synthesis of trans-2-(4-chloro-3-fluorophenoxy)-N-(4-(4-(4-chlorophenyl)thiazol-2-yl)cyclohexyl)acetamide

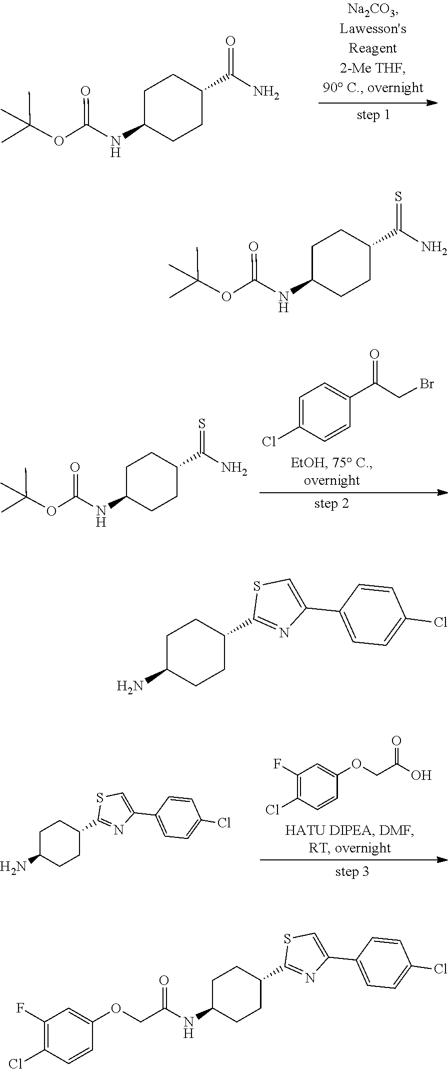

### Step 1: Synthesis of trans-tert-butyl (4-carbamothioylcyclohexyl)carbamate

To a stirred solution of trans-tert-butyl (4-carbamoylcyclohexyl)carbamate (0.400 g, 1.64 mmol, 1.0 eq.) in methyl-THF (8.0 mL) was added Lawesson's reagent (2.65 g, 6.56 mmol, 4.0 eq.), Na₂CO₃ (0.347 g, 3.28 mmol, 2.0 eq.). The reaction mixture was heated at 90° C. overnight. Product formation was confirmed by LCMS and TLC. After completion of reaction, the reaction mixture was quenched with saturated sodium bicarbonate solution and extracted with ethyl acetate (30 mL×3). Combined organic layer was washed with water (3×20 mL), dried over anhydrous Na₂SO₄ and concentrated under reduced pressure to obtain trans-tert-butyl (4-carbamothioylcyclohexyl)carbamate (0.350 g, 85% yield) as an off white solid. LCMS 259.14 [M+H]⁺.

### Step 2: Synthesis of trans-4-(4-(4-chlorophenyl)thiazol-2-yl)cyclohexanamine

Trans-tert-butyl (4-carbamothioylcyclohexyl)carbamate (0.100 g, 0.387 mmol, 1.0 eq.) and 2-bromo-1-(4-chlorophenyl)ethanone (0.108 g, 0.464 mmol, 1.2 eq.) were dissolved in ethanol (4 ml) and heated at 75° C. overnight. Product formation was confirmed by LCMS and TLC. After completion of reaction, the reaction mixture was concentrated under reduced pressure. The crude product was purified by flash chromatography (20-30% ethyl acetate in hexane as an eluent) to obtain trans-4-(4-(4-chlorophenyl)thiazol-2-yl)cyclohexanamine (0.050 g, 44% yield) as a white solid. LCMS 293.08 [M+H]⁺; ¹H NMR (400 MHz, DMSO-d₆) δ 8.06 (s, 1H) 7.96 (d, J=8.33 Hz, 4H) 7.38-7.57 (m, 2H) 2.99-3.15 (m, 2H) 2.19 (d, J=10.96 Hz, 2H) 2.06 (d, J=10.52 Hz, 2H) 1.42-1.70 (m, 4H).

### Step 3: Synthesis of trans-2-(4-chloro-3-fluorophenoxy)-N-(4-(4-(4-chlorophenyl)thiazol-2-yl)cyclohexyl)acetamide

To a stirred solution of trans-4-(4-(4-chlorophenyl)thiazol-2-yl)cyclohexanamine (0.050 g, 0.171 mmol, 1 eq.) and 2-(4-chloro-3-fluorophenoxy)acetic acid (0.052 g, 0.256 mmol, 1.5 eq.) in DMF (1.0 mL) was added HATU (0.129 g, 0.332 mmol, 2.0 eq.) and DIPEA (0.094 mL, 0.513 mmol, 3.0 eq.). The resulting reaction mixture was stirred at RT overnight. Product formation was confirmed by TLC and LCMS. After completion of reaction, the reaction mixture was poured into water (4 ml) extracted with ethyl acetate (5 mL×3). The combined organic layers were washed with water (5 mL×2), dried over anhydrous Na₂SO₄ and concentrated under reduced pressure. The crude product was purified by flash chromatography (5-15% ethyl acetate in hexane as an eluent) to obtain trans-2-(4-chloro-3-fluorophenoxy)-N-(4-(4-(4-chlorophenyl)thiazol-2-yl)cyclohexyl)acetamide (Compound 34-0.014 g, 17% yield) as a white solid. LCMS 482.08 [M+H]⁺; ¹H NMR (400 MHz, DMSO-d₆) δ 8.01-8.09 (m, 2H) 7.97 (d, J=8.77 Hz, 2H) 7.45-7.54 (m, 3H) 7.08 (dd, J=11.62, 2.85 Hz, 1H) 6.86 (dd, J=8.77, 2.19 Hz, 1H) 4.52 (s, 2H) 3.63-3.80 (m, 1H) 2.94-3.08 (m, 1H) 2.18 (d, J=12.72 Hz, 2H) 1.90 (d, J=10.09 Hz, 2H) 1.53-1.69 (m, 2H) 1.38-1.53 (m, 2H).

### Example 34

### Synthesis of 2-(4-chloro-3-fluorophenoxy)-N-(4-(5-(4-chlorophenyl)-1,3,4-oxadiazol-2-yl)piperidin-1-yl)acetamide

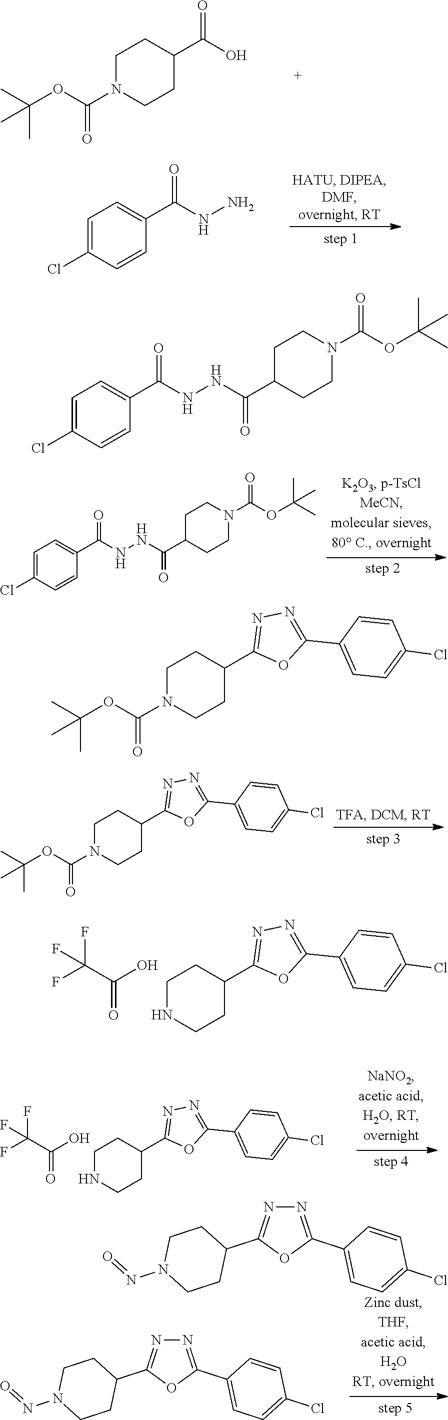

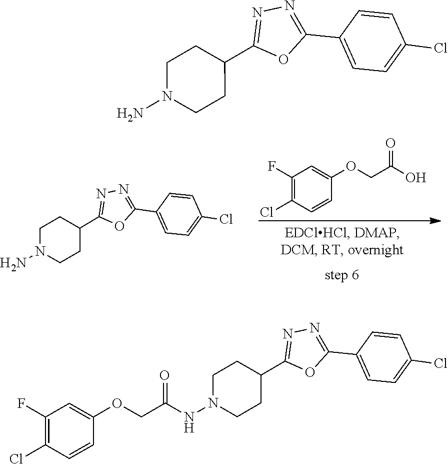 [SMILES: NN1CCC(c2nnc(-c3ccc(Cl)cc3)o2)CC1 ; NN1CCC(c2nnc(-c3ccc(Cl)cc3)o2)CC1 ; O=C(O)COc1ccc(Cl)c(F)c1 ; O=C(COc1ccc(Cl)c(F)c1)NN1CCC(c2nnc(-c3ccc(Cl)cc3)o2)CC1]

### Step 1: Synthesis of tert-butyl 4-(2-(4-chlorobenzoyl)hydrazinecarbonyl)piperidine-1-carboxylate

To a stirred solution of 4-chlorobenzohydrazide (1.00 g, 5.88 mmol, 1.0 eq.), 1-(tert-butoxycarbonyl)piperidine-4-carboxylic acid (1.6 g, 7.05 mmol, 1.2 eq.) and HATU (4.4 g, 11.76 mmol, 2.0 eq.) in DMF (5 mL) was added DIPEA (1.5 g, 11.76 mmol, 2.0 eq.). The resulting reaction mixture was stirred at RT overnight. Product formation was confirmed by LCMS. After completion of reaction, the reaction mixture was poured into ice cold water (10 ml). The resulting solid was filtered off and dried under vacuum to obtain tert-butyl 4-(2-(4-chlorobenzoyl)hydrazinecarbonyl)piperidine-1-carboxylate (1.2 g, 53.5% yield) as an off white solid. LCMS 382.2[M+H]⁺; ¹H NMR (400 MHz, DMSO-d₆) δ 10.41 (s, 1H) 9.93 (s, 1H) 7.88 (m, J=8.33 Hz, 2H) 7.58 (m, J=8.77 Hz, 2H) 3.96 (d, J=12.28 Hz, 2H) 2.79 (br. s., 2H) 2.68 (d, J=7.45 Hz, 2H) 2.50-2.40 (m, 1H), 1.72 (d, J=10.96 Hz, 2H) 1.31-1.60 (m, 9H).

### Step 2: Synthesis of tert-butyl 4-(5-(4-chlorophenyl)-1,3,4-oxadiazol-2-yl)piperidine-1-carboxylate

To a stirred mixture of tert-butyl 4-(2-(4-chlorobenzoyl)hydrazinecarbonyl)piperidine-1-carboxylate (1.00 g, 2.62 mmol, 1.0 eq.), K₂CO₃ (1.0 g, 7.87 mmol, 3.0 eq.) and molecular sieves (1.0 g) in acetonitrile (20 mL) was added 4-toluenesulfonyl chloride (1.2 g, 6.56 mmol, 1.0 eq.) and the resulting reaction mixture was heated at 80° C. overnight. Product formation was confirmed by LCMS and TLC. After completion of reaction, the reaction mixture filtered and filtrate was diluted with EtOAc (25 mL). Organic layer was washed with water (20 mL) and brine (20 mL), dried over anhydrous Na₂SO₄ and concentrated. The crude product was purified by flash chromatography to obtain tert-butyl 4-(5-(4-chlorophenyl)-1,3,4-oxadiazol-2-yl)piperidine-1-carboxylate (0.900 g, 94.5% yield) as off white solid. LCMS 364.3 [M+H]⁺; ¹H NMR (400 MHz, DMSO-d₆) δ 8.01 (d, J=8.77 Hz, 2H) 7.67 (d, J=8.33 Hz, 2H) 3.93 (d, J=13.59 Hz, 2H) 3.13-3.30 (m, 1H) 3.00 (br. s., 2H) 2.06 (d, J=13.59 Hz, 2H) 1.99-1.82 (m, 2H) 1.31-1.54 (m, 9H).

### Step 3: Synthesis of 2-(4-chlorophenyl)-5-(piperidin-4-yl)-1,3,4-oxadiazole 2,2,2-trifluoroacetate

To a stirred solution of tert-butyl 4-(5-(4-chlorophenyl)-1,3,4-oxadiazol-2-yl)piperidine-1-carboxylate (1.00 g, 2.75 mmol, 1.0 eq.) in DCM (10 mL) was added TFA (2.0 mL). The resulting reaction mixture was stirred at RT overnight. Product formation was confirmed by LCMS. After completion of reaction, the reaction mixture was concentrated under reduced pressure. The crude product was crystallized in diethyl ether and dried under vacuum to obtain 2-(4-chlorophenyl)-5-(piperidin-4-yl)-1,3,4-oxadiazole 2,2,2-trifluoroacetate (1.0 g, 97% yield) as an off white solid. LCMS 264.2 [M+H]⁺.

### Step 4: Synthesis of 2-(4-chlorophenyl)-5-(I-nitrosopiperidin-4-yl)-1,3,4-oxadiazole

To a stirred solution of 2-(4-chlorophenyl)-5-(piperidin-4-yl)-1,3,4-oxadiazole 2,2,2-trifluoroacetate (1.0 g, 2.65 mmol, 1.0 eq.) and NaNO₂ (1.4 g, 21.22 mmol, 8.0 eq.) in H₂O (10 mL) was added acetic acid (1.0 mL) at RT. The resulting reaction mixture was stirred at room temperature overnight. Product formation was confirmed by LCMS and TLC. After completion of reaction, the reaction mixture was poured into ice cold water (10 mL). The resulting solid was filtered off, washed with water and dried under vacuum to obtain 2-(4-chlorophenyl)-5-(1-nitrosopiperidin-4-yl)-1,3,4-oxadiazole (0.700 g, 96% yield) as a white solid. LCMS 293.2 [M+H]⁺; ¹H NMR (400 MHz, DMSO-d₆) δ 8.02 (m, J=8.33 Hz, 2H) 7.68 (m, J=8.77 Hz, 2H) 4.62 (tt, J=14.03, 4.38 Hz, 2H) 3.93-4.15 (m, 1H) 3.44-3.67 (m, 1H) 3.00-3.26 (m, 1H) 2.25-2.44 (m, 1H) 2.08-2.21 (m, 1H) 1.87-2.08 (m, 1H) 1.60-1.77 (m, 1H).

### Step 5: Synthesis of 4-(5-(4-chlorophenyl)-1,3,4-oxadiazol-2-yl)piperidin-1-amine

To a stirred solution 2-(4-chlorophenyl)-5-(1-nitrosopiperidin-4-yl)-1,3,4-oxadiazole (0.500 g, 1.71 mmol, 1.0 eq.) in DCM (20 mL) and water (20 mL) was added zinc dust (1.1 g, 17.1 mmol, 10.0 eq.) followed by the addition of acetic acid (0.5 mL) at RT. The reaction mixture was stirred at room temperature overnight. Product formation was confirmed by LCMS. After completion of reaction, the reaction mixture was filtered through Celite®. Filtrate was basified with liquid NH₃ and then extracted with DCM (100 ml). Organic extracts were washed with water (10 mL) and brine (10 mL), dried over anhydrous Na₂SO₄ and concentrated under reduced pressure to obtain 4-(5-(4-chlorophenyl)-1,3,4-oxadiazol-2-yl)piperidin-1-amine (0.370 g, 77% yield) as off white solid. LCMS 279.2 [M+H]⁺.

### Step 6: Synthesis of 2-(4-chloro-3-fluorophenoxy)-N-(4-(5-(4-chlorophenyl)-1,3,4-oxadiazol-2-yl)piperidin-1-yl)acetamide

To a stirred solution of 4-(5-(4-chlorophenyl)-1,3,4-oxadiazol-2-yl)piperidin-1-amine (0.500 mg, 1.79 mmol, 1.0 eq.), DMAP (262 mg, 2.148 mmol, 1.2 eq.) and EDCl.HCl (515 mg, 2.68 mmol, 1.5 eq.) in DCM (20 mL) was added 2-(4-chloro-3-fluorophenoxy)acetic acid (440 mg, 2.15 mmol, 1.2 eq.). The resulting reaction mixture was stirred at RT overnight. Product formation was confirmed by LCMS. After completion of reaction, the reaction mixture was concentrated under reduced pressure. The resulting residue was dissolved in water and extracted with ethyl acetate (25 mL×2). Combined organic layer was washed with brine, dried over Na₂SO₄ and concentrated under reduced pressure. The crude product was purified by flash chromatography (0-5% MeOH in DCM as an eluent) which was further purified by reverse phase HPLC to obtain 2-(4-chloro-3-fluorophenoxy)-N-(4-(5-(4-chlorophenyl)-1,3,4-oxadiazol-2-yl)piperidin-1-yl)acetamide (Compound 35-0.100 g, 12% yield) as an white solid. LCMS 465.4 [M+H]⁺; ¹H NMR (400 MHz, DMSO-d₆) δ 9.25 (s, 1H) 8.90 (s, 1H) 8.01 (d, J=8.33 Hz, 2H) 7.60-7.75 (m, 1H) 7.36-7.58 (m, 1H) 7.04 (ddd, J=19.95, 11.40, 2.85 Hz, 1H) 6.70-6.98 (m, 1H) 4.93 (s, 1H) 4.49 (s, 1H) 3.17 (br. s., 1H) 3.06-3.14 (m, 1H) 2.92-2.92 (m, 1H) 2.67 (d, J=1.32 Hz, 1H) 2.60 (br. s., 1H) 2.02-2.28 (m, 2H) 1.79-2.02 (m, 2H).

### Example 35

### Synthesis of cis-2-(4-chloro-3-fluorophenoxy)-N-(4-(1-(4-chlorophenyl)-1H-pyrazol-4-yl)cyclohexyl)acetamide and trans-2-(4-chloro-3-fluorophenoxy)-N-(4-(1-(4-chlorophenyl)-1H-pyrazol-4-yl)cyclohexyl)acetamide

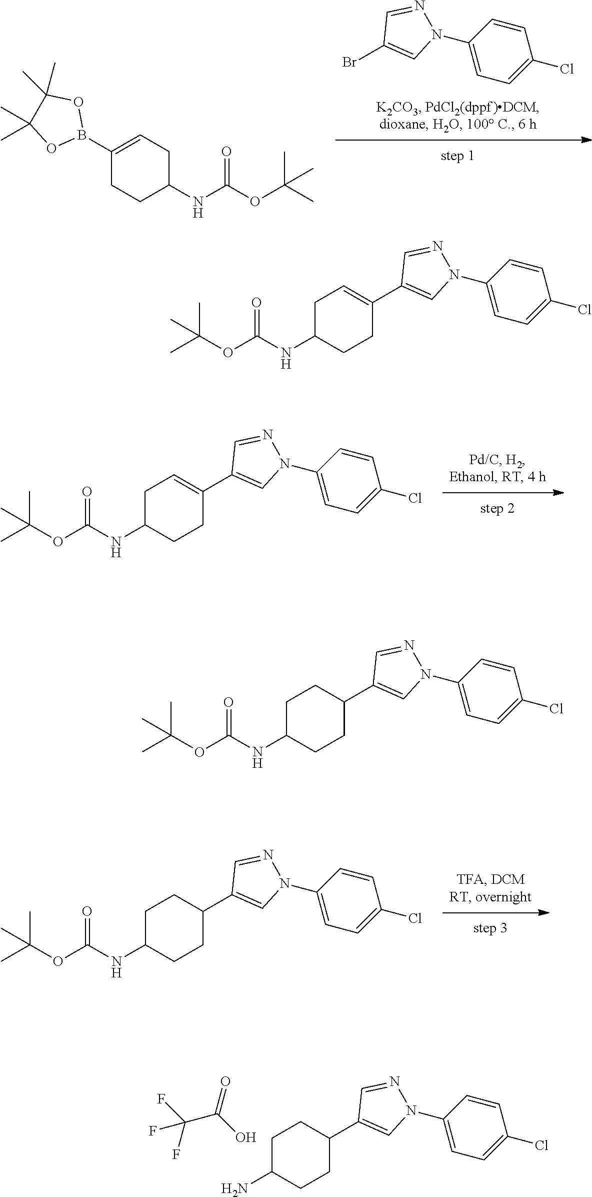

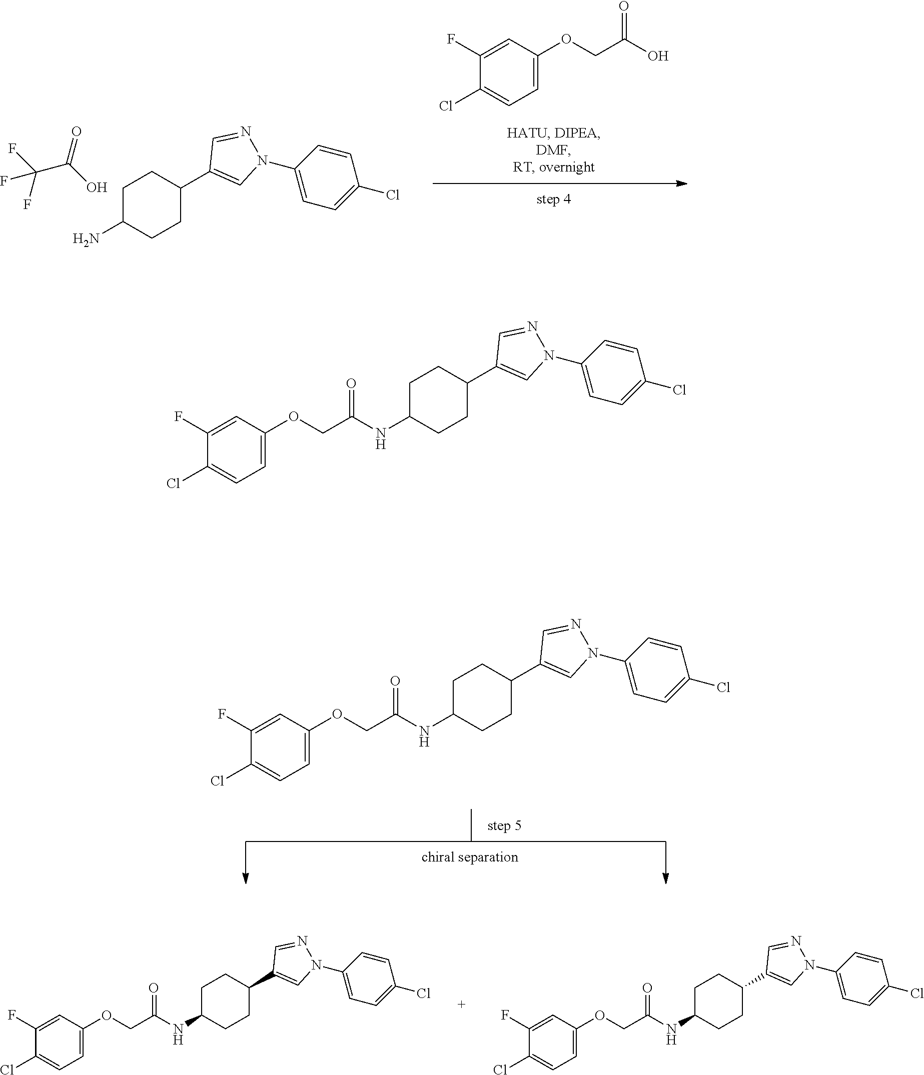

### Step 1: Synthesis of tert-butyl (4-(1-(4-chlorophenyl)-1H-pyrazol-4-yl)cyclohex-3-en-1-yl)carbamate

To a stirred solution of tert-butyl (4-(4,4,5,5-tetramethyl-1,3,2-dioxaborolan-2-yl)cyclohex-3-en-1-yl)carbamate (500 mg, 1.547 mmol, 1 eq.), 4-bromo-1-(4-chlorophenyl)-1H-pyrazole (398 mg, 1.547 mmol, 1 eq.) and K₂CO₃ (427 mg, 3.09 mmol, 2.0 eq.) in dioxane (20 mL) and water (5 mL) was added PdCl₂ (dppf).DCM (126 mg, 1.547 mmol, 0.1 eq.). The resulting reaction mixture was refluxed at 100° C. for 6 hours. Product formation was confirmed by LCMS and TLC. After completion of reaction, the reaction mixture was filtered through Celite® and washed with ethyl acetate (2×20 mL). The organic layer was concentrated under reduced pressure. The crude product was enriched by flash chromatography (0-20% ethyl acetate in hexane as an eluent) to obtain tert-butyl (4-(1-(4-chlorophenyl)-1H-pyrazol-4-yl)cyclohex-3-en-1-yl)carbamate (250 mg, 43% yield) as a white solid. LCMS 374.3 [M+H]⁺; ¹H NMR (400 MHz, DMSO-d₆) δ 8.53 (s, 1H), 7.91 (s, 1H), 7.89-7.71 (m, J=9.2 Hz, 2H), 7.61-7.45 (m, J=8.8 Hz, 2H), 6.83 (d, J=6.6 Hz, 1H), 6.05 (br. s., 1H), 3.51 (br. s., 1H), 2.43-2.28 (m, 1H), 2.05 (br. s., 1H), 1.88 (br. s., 2H), 1.54 (d, J=6.6 Hz, 2H), 1.42-1.29 (m, 9H).

### Step 2: Synthesis of tert-butyl (4-(1-(4-chlorophenyl)-1H-pyrazol-4-yl)cyclohexyl)carbamate

To a stirred solution of tert-butyl (4-(1-(4-chlorophenyl)-1H-pyrazol-4-yl)cyclohex-3-en-1-yl)carbamate (100 mg, 0.268 mmol, 1 eq.) in ethanol (15 mL) was added Pd/C (20 mg, 10% Pd on carbon) under N₂. The reaction mixture was degassed under vacuum and purged with H₂ gas at RT for 1 h. Product formation was confirmed by LCMS. The reaction mixture was filtered through Celite® and concentrated under reduced pressure to obtain tert-butyl (4-(1-(4-chlorophenyl)-1H-pyrazol-4-yl)cyclohexyl)carbamate (50 mg, 50% yield) as a white solid. LCMS 376.3 [M+H]⁺.

### Step 3: Synthesis of 4-(1-(4-chlorophenyl)-1H-pyrazol-4-yl)cyclohexanamine 2,2,2-trifluoroacetate

To a solution of tert-butyl 4-(1-(4-chlorophenyl)-1H-pyrazol-4-yl)cyclohexylcarbamate (110 mg, 0.293 mmol, 1 eq.) in DCM (10 mL) was added TFA (0.2 mL) under N₂. The reaction mixture was stirred at RT for 2 h. Product formation was confirmed by LCMS. The reaction mixture was concentrated under reduced pressure. The crude product was crystallized in diethyl ether to obtain 4-(1-(4-chlorophenyl)-1H-pyrazol-4-yl)cyclohexanamine 2,2,2-trifluoroacetate (110 mg, 96% yield) as a white solid. LCMS 276.2 [M+H]⁺.

### Step 4: Synthesis of 2-(4-chloro-3-fluorophenoxy)-N-(4-(1-(4-chlorophenyl)-1H-pyrazol-4-yl)cyclohexyl)acetamide

To a stirred solution of 4-(1-(4-chlorophenyl)-1H-pyrazol-4-yl)cyclohexanamine 2,2,2-trifluoroacetate (110 mg, 0.282 mmol, 1 eq.), 2-(4-chloro-3-fluorophenoxy)acetic acid (58 mg, 0.284 mmol, 1 eq.) and HATU (161 mg, 0.423 mmol, 1.5 eq.) in DMF (5 mL) was added DIPFA (0.2 mL, 1.13 mmol, 4.0 eq.). The resulting reaction mixture was stirred at RT for 4 hours. Product formation was confirmed by LCMS. After completion of reaction, the reaction mixture was diluted with ice cold water (20 mL) and extracted with ethyl acetate (2×10 mL). The organic layer was washed with cold water (5×10 mL) and brine (10 mL), dried over anhydrous Na₂SO₄, and concentrated under reduced pressure. The crude product was purified by reverse phase HPLC to obtain 2-(4-chloro-3-fluorophenoxy)-N-(4-(1-(4-chlorophenyl)-1H-pyrazol-4-yl)cyclohexyl)acetamide (30 mg, 23% yield) as a white solid. LCMS 462.4 [M+H]⁺; ¹H NMR (400 MHz, DMSO-d₆) δ 8.37-8.30 (m, 1H), 7.99 (d, J=7.9 Hz, 1H), 7.92-7.77 (m, 2H), 7.70-7.63 (m, 1H), 7.57-7.35 (m, 3H), 7.11-7.01 (m, 1H), 6.90-6.78 (m, 1H), 4.59-4.44 (s, 2H), 3.87-3.67 (br. s., 1H), 2.75 (br. s., 1H), 1.99 (br. s., 2H), 1.91-1.74 (m, 2H), 1.60 (d, J=5.0 Hz, 2H), 1.47-1.31 (m, 2H).

### Step 5: Chiral Separation

The enantiomers cis-2-(4-chloro-3-fluorophenoxy)-N-(4-(1-(4-chlorophenyl)-1H-pyrazol-4-yl)cyclohexyl)acetamide (Compound 36—[α]D20=NA; elution time: 17.92 min) and trans-2-(4-chloro-3-fluorophenoxy)-N-(4-(1-(4-chlorophenyl)-1H-pyrazol-4-yl)cyclohexyl)acetamide (Compound 37—[α]D20=NA; elution time: 39.60 min), were separated by chiral SFC (Chiralpak-IG, 250×21 mm, 5μ), using an isocratic program with analytical grade liquid carbon dioxide, HPLC grade isopropyl alcohol and HPLC grade hexane (0.2% DEA). LCMS: 462.4 [M+H]⁺.

### Example 36

### Synthesis of trans-N-(6-chloroquinolin-2-yl)-4-(5-((cis-3-(trifluoromethoxy)cyclobutoxy)methyl)-1,3,4-oxadiazol-2-yl)cyclohexanecarboxamide

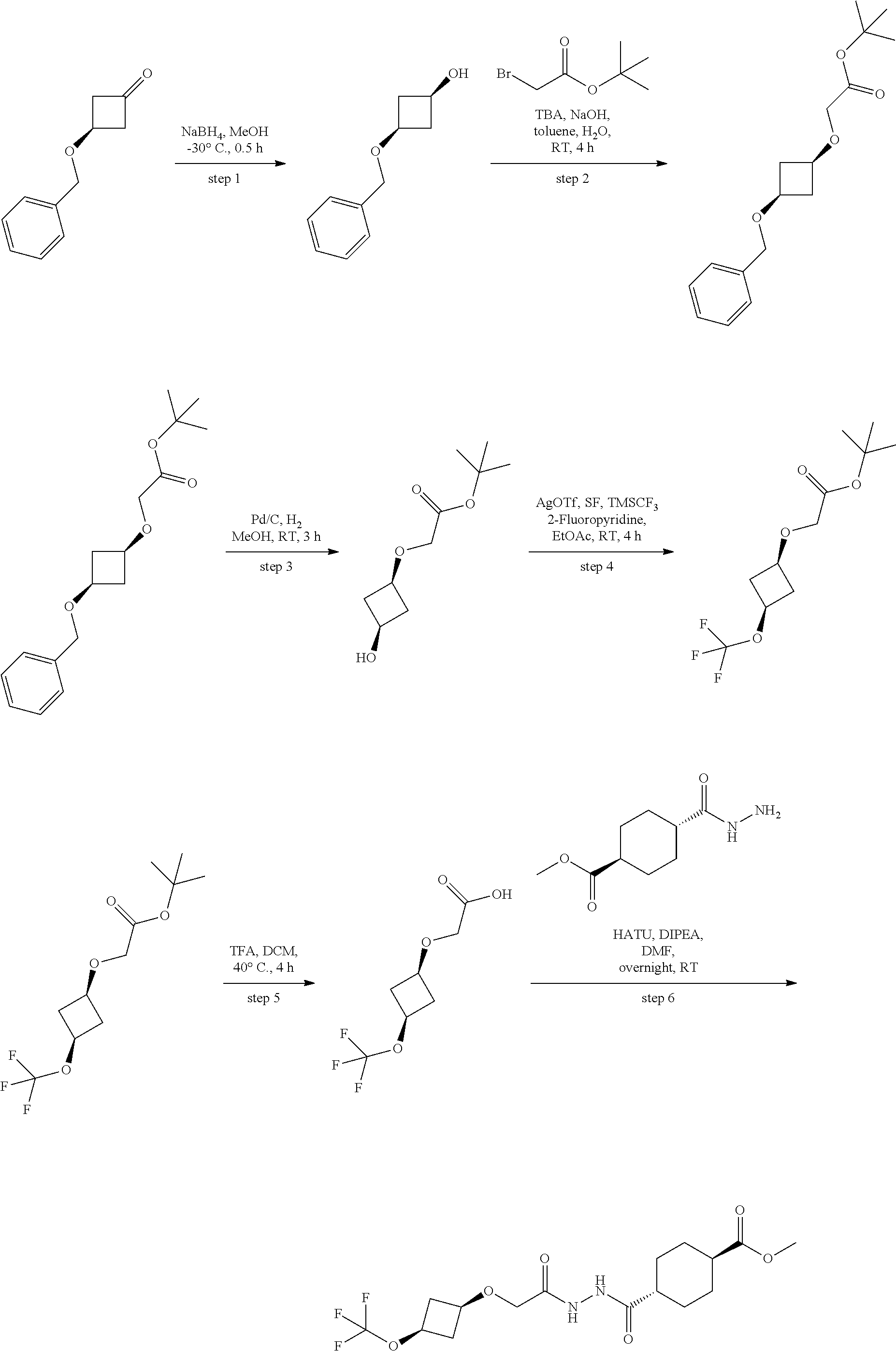

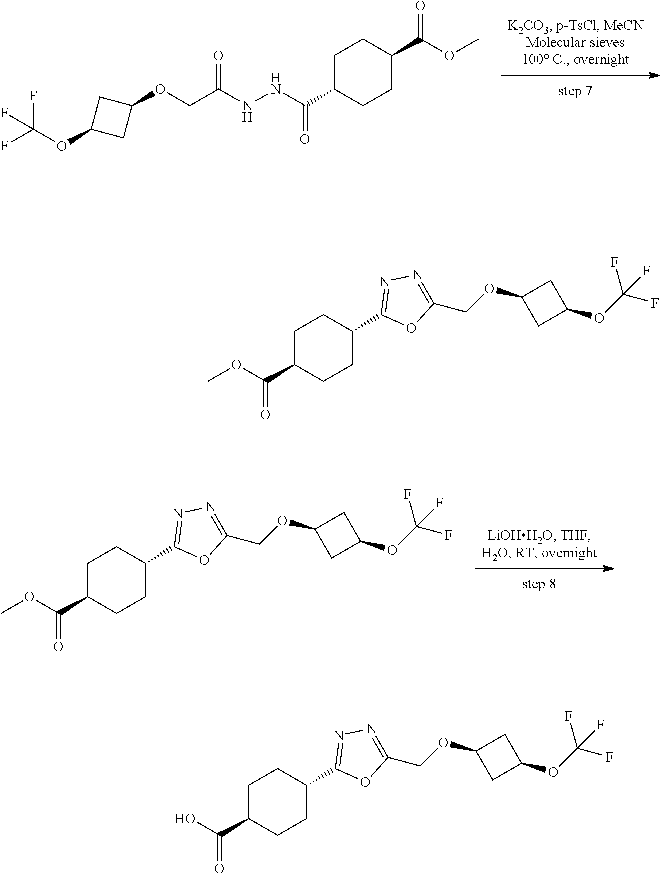

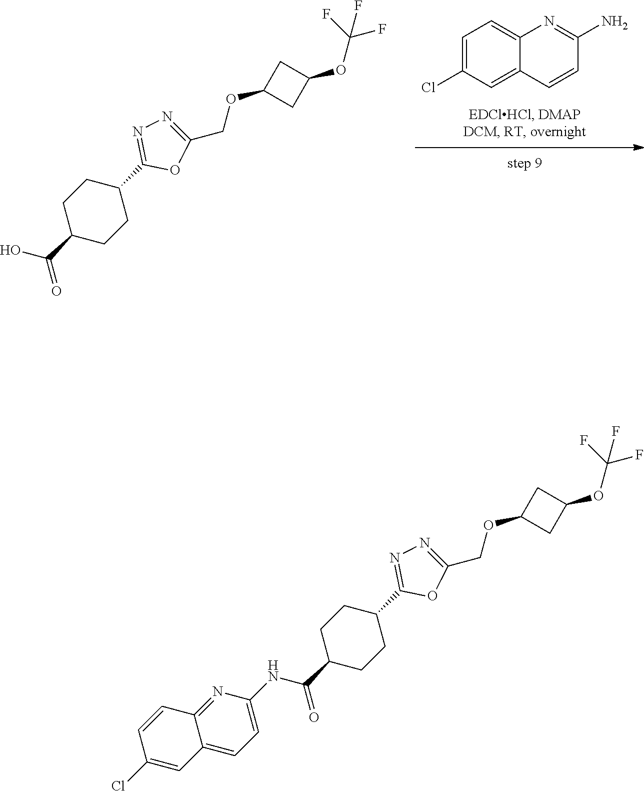 [SMILES: O=C(O)[C@H]1CC[C@H](c2nnc(CO[C@H]3C[C@@H](OC(F)(F)F)C3)o2)CC1 ; Nc1ccc2cc(Cl)ccc2n1 ; O=C(Nc1ccc2cc(Cl)ccc2n1)[C@H]1CC[C@H](c2nnc(CO[C@H]3C[C@@H](OC(F)(F)F)C3)o2)CC1]

### Step 1: Synthesis of cis-3-(benzyloxy)cyclobutanol

To a solution of 3-benzyloxycyclobutanone (1000 mg, 5.68 mmol, 1 eq.) in MeOH (20 mL) was added NaBH₄ (2148 mg, 5.68 mmol, 1 eq.) at −30° C. under N₂ over 2 h. The reaction mixture was stirred at −30° C. for 0.5 h. after completion of reaction the reaction mixture was quenched by the addition of ice followed by saturated NH₄Cl solution (10 mL) slowly at 0° C. The MeOH was removed under reduced pressure and the aqueous layer was extracted with EtOAc (3×20 mL). The combined organic layers were washed with brine (20 mL), dried over anhydrous Na₂SO₄, filtered, and concentrated under reduced pressure to obtain cis-3-(benzyloxy)cyclobutanol (1000 mg, 98% yield) as a transparent oil. ¹H NMR (400 MHz, CDCL₃) δ 7.32-7.18 (m, 5H), 4.35 (s, 2H), 3.83 (quin, J=7.17 Hz, 1H), 3.56 (quin, J=6.95 Hz, 1H), 2.69-2.60 (m, 2H), 1.91-1.82 (m, 2H).

### Step 2: Synthesis of cis-tert-butyl 2-(3-(benzyloxy)cyclobutoxy)acetate

To a solution of cis-3-(benzyloxy)cyclobutanol (1000 mg, 5.62 mmol, 1 eq.), tert-butyl 2-bromoacetate (1643 mg, 8.426 mmol, 1.5 eq.), tetrabutylammonium hydrogen sulfate (95 mg, 0.28 mmol, 0.05 eq.) in toluene (30 mL) was added NaOH (3146 mg, 78.65 mmol, 14 eq.) in water (10 mL). The reaction mixture was stirred at 25° C. for 4 h. After completion of reaction the ice-cold water (40 mL) was added and extracted with ethyl acetate (3×20 mL). The combined organic layers were washed with brine (100 mL), dried over anhydrous Na₂SO₄ and concentrated under reduced pressure to obtain cis-tert-butyl 2-(3-(benzyloxy)cyclobutoxy)acetate (1500 mg, 91% yield) as a transparent oil. ¹H NMR (400 MHz, CDCL₃) δ 7.39-7.27 (m, 5H), 4.42 (s, 2H), 3.89 (s, 2H), 3.75-3.62 (m, 2H), 2.65 (dtd, J 9.26, 6.28, 6.28, 3.31 Hz, 2H), 2.09-2.00 (m, 2H), 1.48 (s, 9H).

### Step 3: Synthesis of cis-tert-butyl 2-(3-hydroxycyclobutoxy)acetate

To a stirred solution of cis-tert-butyl 2-(3-(benzyloxy)cyclobutoxy)acetate (1500 mg, 5.137 mmol, 1 eq.) in MeOH (20 mL) was added Pd/C (150 mg, 10% Pd on carbon) under N₂. The reaction mixture was degassed under vacuum and then purged with H₂ gas at RT for 6 h. The reaction mixture was filtered through Celite® and concentrated under reduced pressure to obtain cis-tert-butyl 2-(3-hydroxycyclobutoxy)acetate (950 mg, 91.6%) as a transparent oil. ¹H NMR (400 MHz, CDCL₃) δ 3.94-3.89 (m, 1H), 3.88 (s, 2H), 3.67 (quin, J 6.89 Hz, 1H), 2.78-2.69 (m, 2H), 2.01-1.92 (m, 2H), 1.80 (br d, J=6.39 Hz, 1H), 1.47 (s, 9H).

### Step 4: Synthesis of cis-tert-butyl 2-(3-(trifluoromethoxy)cyclobutoxy)acetate

To a round bottom flask covered with aluminium foil in a water bath were added AgOTf (3.6 g, 14.11 mmol, 3.0 eq.), Selectfluor (2.5 g, 7.054 mmol, 1.5 eq.), KF (1.1 g, 18.812 mmol, 4.0 eq.), and cis-tert-butyl 2-(3-hydroxycyclobutoxy)acetate (0.95 g, 4.703 mmol, 1.0 equiv) under N₂. And then EtOAc (40 mL), 2-fluoropyridine (1.37 g, 14.11 mmol, 3.0 equiv) and TMSCF₃ (2.0 g, 14.11 mmol, 3.0 eq.) were added drop wise successively while keeping the inner temperature below 30° C. The reaction mixture was stirred at 25° C. for 16 h. The reaction mixture was filtered through Celite® and the filtrate was concentrated under reduced pressure. The residue was purified by flash chromatography (0-5% ethyl acetate in hexane as an eluent) to obtain cis-tert-butyl 2-(3-(trifluoromethoxy)cyclobutoxy)acetate (540 mg, 42% yield) as a transparent oil. ¹H NMR (400 MHz, CDCL₃) δ 3.94-3.89 (m, 1H), 3.88 (s, 2H), 3.67 (quin, J 6.89 Hz, 1H), 2.78-2.69 (m, 2H), 2.01-1.92 (m, 2H), 1.80 (br d, J=6.39 Hz, 1H), 1.47 (s, 9H).

### Step 5: Synthesis of cis-2-(3-(trifluoromethoxy)cyclobutoxy)acetic acid

To a solution of cis-tert-butyl 2-(3-(trifluoromethoxy)cyclobutoxy)acetate (540 mg, 2.0 mmol, 1.0 eq.) in DCM (100 mL) was added TFA (0.5 mL) under N₂. The reaction mixture was stirred at 40° C. for 2 h. The reaction mixture was concentrated under reduced pressure. The residue was dissolved in DCM (10 mL), washed with H₂O (3×5 mL), dried over Na₂SO₄, filtered and concentrated under reduced pressure to obtain cis-2-(3-(trifluoromethoxy)cyclobutoxy)acetic acid (475 mg, 99% yield) as a yellow oil. ¹H NMR (400 MHz, CDCL₃) δ 4.26-4.19 (m, 1H), 4.00 (s, 2H), 3.73-3.70 (m, 1H), 2.77-2.74 (m, 2H), 2.27-2.24 (m, 2H).

### Step 6: Synthesis of trans-methyl 4-(2-(2-(cis-3-(trifluoromethoxy)cyclobutoxy)acetyl)hydrazinecarbonyl)cyclohexanecarboxylate

To a stirred mixture of cis-2-(3-(trifluoromethoxy)cyclobutoxy)acetic acid (200 mg, 0.935 mmol, 1 eq.), trans-methyl 4-(hydrazinecarbonyl)cyclohexanecarboxylate (224 mg, 1.12 mmol, 1.2 eq.) and HATU (532 mg, 1.402 mmol, 1.5 eq.) in DMF (7 mL) was added DIPEA (482 mg, 3.74 mmol, 4.0 eq.) and the resulting reaction mixture was stirred at RT overnight. Product formation was confirmed by LCMS. After completion of reaction, the reaction mixture was poured into ice cold water (50 ml). The resulting off white solid was filtered off and dried under vacuum to obtain trans-methyl 4-(2-(2-(cis-3-(trifluoromethoxy)cyclobutoxy)acetyl)hydrazinecarbonyl)cyclohexanecarboxylate (267 mg, 74%) as an off white solid. LCMS 397.3 [M+H]⁺; ¹H NMR (400 MHz, DMSO-d₆) δ 9.67 (s, 1H), 9.71 (s, 1H), 4.56-4.40 (m, 1H), 3.96-3.81 (m, 2H), 3.74 (td, J=6.9, 13.4 Hz, 1H), 3.59 (s, 3H), 2.82-2.62 (m, 1H), 2.37-2.25 (m, 1H), 2.24-2.09 (m, 2H), 2.01-1.86 (m, 2H), 1.84-1.65 (m, 2H), 1.48-1.19 (m, 6H).

### Step 7: Synthesis of trans-methyl 4-(5-((cis-3-(trifluoromethoxy)cyclobutoxy)methyl)-1,3,4-oxadiazol-2-yl)cyclohexanecarboxylate

To a stirred mixture of trans-methyl 4-(2-(2-(cis-3-(trifluoromethoxy)cyclobutoxy)acetyl)hydrazinecarbonyl)cyclohexanecarboxylate (250 mg, 0.631 mmol, 1.0 eq.), K₂CO₃ (435 mg, 3.16 mmol, 5.0 eq.) and molecular sieves (500 mg) in acetonitrile (50 mL) was added 4-toluenesulfonyl chloride (300 mg, 1.59 mmol, 2.5 eq.) and the resulting reaction mixture was stirred at 90° C. overnight. Product formation was confirmed by LCMS. After completion of reaction, the reaction mixture was filtered off and the filtrate was diluted with EtOAc (100 mL). The organic layer was washed with saturated NaHCO₃ (50 mL) and brine (50 mL), dried over anhydrous Na₂SO₄ and concentrated under reduced pressure to obtain trans-methyl 4-(5-((cis-3-(trifluoromethoxy)cyclobutoxy)methyl)-1,3,4-oxadiazol-2-yl)cyclohexanecarboxylate (208 mg, 87% yield) as an off white solid. LCMS 379.3 [M+H]⁺; ¹H NMR (400 MHz, DMSO-d₆) δ 4.63 (s, 2H), 4.50-4.39 (m, 1H), 3.79 (td, J=6.8, 13.6 Hz, 1H), 3.67-3.39 (m, 3H), 3.04-2.83 (m, 1H), 2.79-2.61 (m, 2H), 2.40 (br. s., 1H), 2.16-2.03 (m, 3H), 2.03-1.73 (m, 3H), 1.62-1.38 (m, 4H).

### Step 8: Synthesis of trans-4-(5-((cis-3-(trifluoromethoxy)cyclobutoxy)methyl)-1,3,4-oxadiazol-2-yl)cyclohexanecarboxylic acid

To a stirred solution of trans-methyl 4-(5-((cis-3-(trifluoromethoxy)cyclobutoxy)methyl)-1,3,4-oxadiazol-2-yl)cyclohexanecarboxylate (250 mg, 0.66 mmol, 1.0 eq.) in THE (10 mL) and H₂O (10 mL) was added LiOH.H₂O (83 mg, 1.98 mmol, 3.0 eq.) and the resulting reaction mixture was stirred at RT for overnight under nitrogen atmosphere. Product formation was confirmed by LCMS. After completion of reaction, the reaction mixture was concentrated under reduced pressure. Residue was dissolved in H₂O (10 mL) and extracted with ethyl acetate (10 mL). The aqueous layer was acidified with 6 M HCl (pH˜4 to 5), the resulting solid was filtered off and dried under vacuum to obtain trans-4-(5-((cis-3-(trifluoromethoxy)cyclobutoxy)methyl)-1,3,4-oxadiazol-2-yl)cyclohexanecarboxylic acid (200 mg, 83% yield) as an off white solid. LCMS 365.4 [M+H]⁺; ¹H NMR (400 MHz, DMSO-d₆) δ 12.13 (s, 1H), 4.63 (s, 2H), 4.49 (quin, J=7.2 Hz, 1H), 3.79 (td, J=7.0, 13.7 Hz, 1H), 2.95 (t, J=11.8 Hz, 1H), 2.76-2.63 (m, 2H), 2.35-2.17 (m, 1H), 2.15-1.88 (m, 6H), 1.60-1.38 (m, 4H).

### Step 9: Synthesis of trans-N-(6-chloroquinolin-2-yl)-4-(5-((cis-3-(trifluoromethoxy)cyclobutoxy)methyl)-1,3,4-oxadiazol-2-yl)cyclohexanecarboxamide

To a stirred solution of trans-4-(5-((cis-3-(trifluoromethoxy)cyclobutoxy)methyl)-1,3,4-oxadiazol-2-yl)cyclohexanecarboxylic acid (50 mg, 0.137 mmol, 1 eq.) in DCM (20 mL) was added EDCI.HCl (120 mg, 0.625 mmol, 4.6 eq.) and DMAP (60 mg, 0.491 mmol, 3.6 eq.) and stirred at RT for 15 minutes followed by the addition of 6-chloroquinolin-2-amine (25 mg, 0.137 mmol, 1 eq.). The resulting reaction mixture was stirred at RT overnight. Product formation was confirmed by LCMS and TLC. After completion of reaction, the reaction mixture was diluted with DCM (20 mL). The organic layer was washed with saturated citric acid solution (10 mL) and brine (10 mL), dried over anhydrous Na₂SO₄ and concentrated under reduced pressure. The crude product was purified by flash chromatography (0-2% methanol in DCM as an eluent) to obtain trans-N-(6-chloroquinolin-2-yl)-4-(5-((cis-3-(trifluoromethoxy)cyclobutoxy)methyl)-1,3,4-oxadiazol-2-yl)cyclohexanecarboxamide (Compound 38-13 mg, 18% yield) as a white solid. LCMS 525.6 [M+H]⁺; ¹H NMR (400 MHz, DMSO-d₆) δ 10.85 (s, 1H), 8.39-8.18 (m, 2H), 8.05 (d, J=2.2 Hz, 1H), 7.81 (d, J=9.2 Hz, 1H), 7.71 (dd, J=2.2, 9.2 Hz, 1H), 4.72-4.59 (m, 2H), 4.53-4.40 (m, 1H), 3.81 (td, J=6.6, 13.5 Hz, 1H), 3.02 (t, J=11.4 Hz, 1H), 2.77-2.67 (m, 2H), 2.65 (d, J=12.7 Hz, 1H), 2.20-2.03 (m, 4H), 1.99 (d, J=10.1 Hz, 2H), 1.69-1.44 (m, 4H).

### Example 37

### Synthesis of trans-N-(5-chlorobenzo[d]thiazol-2-yl)-4-(5-((cis-3-(trifluoromethoxy)cyclobutoxy)methyl)-1,3,4-oxadiazol-2-yl)cyclohexanecarboxamide

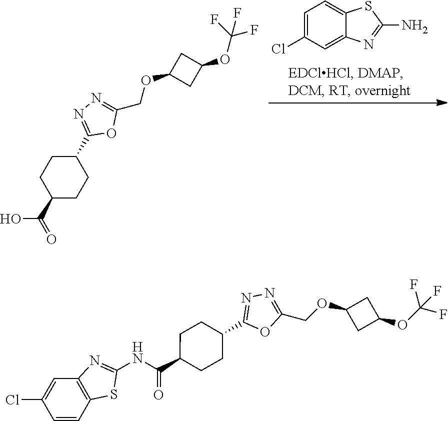 [SMILES: O=C(O)[C@H]1CC[C@H](c2nnc(CO[C@H]3C[C@@H](OC(F)(F)F)C3)o2)CC1 ; Nc1nc2cc(Cl)ccc2s1 ; O=C(Nc1nc2cc(Cl)ccc2s1)[C@H]1CC[C@H](c2nnc(CO[C@H]3C[C@@H](OC(F)(F)F)C3)o2)CC1]

To a stirred solution of trans-4-(5-((cis-3-(trifluoromethoxy)cyclobutoxy)methyl)-1,3,4-oxadiazol-2-yl)cyclohexanecarboxylic acid (50 mg, 0.137 mmol, 1.0 eq.) in DCM (20 mL) was added EDCI.HCl (120 mg, 0.625 mmol, 4.6 eq.) and DMAP (60 mg, 0.491 mmol, 3.6 eq.) and was stirred at RT for 15 minutes followed by addition of 5-chlorobenzo[d]thiazol-2-amine (25 mg, 0.137 mmol, 1.0 eq.). The resulting reaction mixture was stirred at RT overnight. Product formation was confirmed by LCMS and TLC. After completion of reaction, the reaction mixture was diluted with DCM (20 mL). The organic layer was washed with 1 M HCl solution (10 mL) and brine (10 mL), dried over anhydrous Na₂SO₄ and concentrated under reduced pressure. The crude product was purified by flash chromatography (0-40% ethyl acetate in hexane as an eluent) to obtain trans-N-(5-chlorobenzo[d]thiazol-2-yl)-4-(5-((cis-3-(trifluoromethoxy)cyclobutoxy)methyl)-1,3,4-oxadiazol-2-yl)cyclohexanecarboxamide (Compound 39-20 mg, 27% yield) as a white solid. LCMS 531.5 [M+H]⁺; ¹H NMR (400 MHz, DMSO-d₆) δ 12.50 (br. s., 1H), 8.01 (d, J=8.3 Hz, 1H), 7.80 (s, 1H), 7.34 (d, J=8.3 Hz, 1H), 4.64 (s, 2H), 4.50 (td, J=7.2, 14.1 Hz, 1H), 3.80 (td, J=6.8, 13.6 Hz, 1H), 3.02 (t, J=11.4 Hz, 1H), 2.77-2.66 (m, 2H), 2.62 (t, J=11.4 Hz, 1H), 2.16 (d, J=11.8 Hz, 2H), 2.11-1.92 (m, 4H), 1.67-1.41 (m, 4H).

### Example 38

### Synthesis of trans-4-(5-((4-chlorophenoxy)methyl)-1,3,4-oxadiazol-2-yl)-N-(6-chloroquinolin-2-yl)cyclohexanecarboxamide

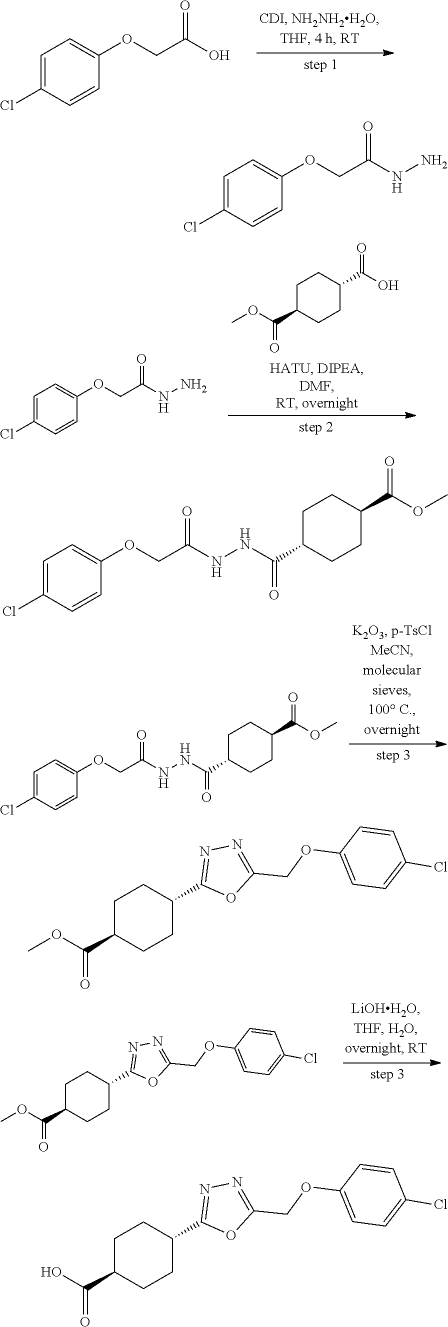

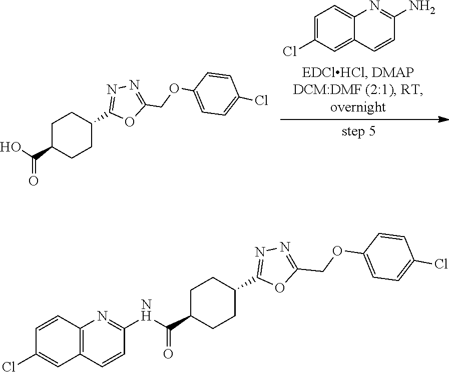 [SMILES: Nc1ccc2cc(Cl)ccc2n1 ; O=C(O)[C@H]1CC[C@H](c2nnc(COc3ccc(Cl)cc3)o2)CC1 ; O=C(Nc1ccc2cc(Cl)ccc2n1)[C@H]1CC[C@H](c2nnc(COc3ccc(Cl)cc3)o2)CC1]

### Step 1: Synthesis of 2-(4-chlorophenoxy)acetohydrazide

To a stirred solution of 2-(4-chlorophenoxy)acetic acid (500 mg, 2.688 mmol, 1.0 eq.) in THF (50 mL) was added CDI (653 mg, 4.032 mmol, 1.5 eq.). The resulting reaction mixture was allowed to stir at RT for 2 hours. Hydrazine hydrate (268 mg, 5.376 mmol, 2.0 eq.) was added to the reaction mixture and stirred at RT for 2 hours. Product formation was confirmed by LCMS. After completion of reaction, the reaction mixture was concentrated under reduced pressure. The resulting solid was washed with diethyl ether and dried under vacuum to obtain 2-(4-chlorophenoxy)acetohydrazide (375 mg, 100% yield) as a white solid. LCMS 201.1 [M+H]⁺.

### Step 2: Synthesis of trans-methyl 4-(2-(2-(4-chlorophenoxy)acetyl)hydrazinecarbonyl)cyclohexanecarboxylate

To a stirred mixture of 2-(4-chlorophenoxy)acetohydrazide (200 mg, 1.0 mmol, 1 eq.), trans-4-(methoxycarbonyl)cyclohexanecarboxylic acid (186 mg, 1.0 mmol, 1.0 eq.) and HATU (570 mg, 1.5 mmol, 1.5 eq.) in DMF (5 mL) was added DIPEA (0.72 mL, 4.0 mmol, 4.0 eq.) and the resulting reaction mixture was stirred at RT overnight. Product formation was confirmed by LCMS. After completion of reaction, the reaction mixture was poured into ice cold water (200 ml). The resulting solid was filtered off and dried under vacuum to obtain trans-methyl 4-(2-(2-(4-chlorophenoxy)acetyl)hydrazinecarbonyl)cyclohexanecarboxylate (300 mg, 81% yield) as a white solid. LCMS 369.3 [M+H]⁺.

### Step 3: Synthesis of trans-methyl 4-(5-((4-chlorophenoxy)methyl)-1,3,4-oxadiazol-2-yl)cyclohexanecarboxylate

To a stirred mixture of trans-methyl 4-(2-(2-(4-chlorophenoxy)acetyl)hydrazinecarbonyl)cyclohexanecarboxylate (300 mg, 0.815 mmol, 1 eq.), K₂CO₃ (562 mg, 4.076 mmol, 5.0 eq.) and molecular sieves (300 mg) in acetonitrile (20 mL) was added 4-toluenesulfonyl chloride (387 mg, 2.04 mmol, 2.5 eq.) and the resulting reaction mixture was stirred at 100° C. overnight. Product formation was confirmed by LCMS. After completion of reaction, the reaction mixture was filtered and the filtrate was diluted with ethyl acetate (100 mL). The organic layer was washed with water (100 mL) and brine (100 mL), dried over anhydrous Na₂SO₄ and concentrated under reduced pressure to obtain trans-methyl 4-(5-((4-chlorophenoxy)methyl)-1,3,4-oxadiazol-2-yl)cyclohexanecarboxylate (250 mg, 87% yield) as an off white solid. LCMS 351.3 [M+H]⁺.

### Step 4: Synthesis of trans-4-(5-((4-chlorophenoxy)methyl)-1,3,4-oxadiazol-2-yl)cyclohexanecarboxylic acid

To a stirred solution of trans-methyl 4-(5-((4-chlorophenoxy)methyl)-1,3,4-oxadiazol-2-yl)cyclohexanecarboxylate (250 mg, 0.679 mmol, 1.0 eq.) in THE (4 mL) and H₂O (4 mL) was added LiOH.H₂O (86 mg, 2.038 mmol, 1.0 eq.) and the resultant reaction mixture was stirred at RT overnight under nitrogen atmosphere. Product formation was confirmed by LCMS. After completion of reaction, the reaction mixture was concentrated under reduced pressure. The residue was dissolved in H₂O (20 mL) and washed with ethyl acetate (20 mL). The aqueous layer was acidified with 6 M HCl (pH˜4 to 5). The resulting solid was filtered off and dried under vacuum to obtain trans-4-(5-((4-chlorophenoxy)methyl)-1,3,4-oxadiazol-2-yl)cyclohexanecarboxylic acid (200 mg, 87% yield) as a white solid. LCMS 337.2 [M+H]⁺; ¹H NMR (400 MHz, DMSO-d₆) δ 12.10 (br. s., 1H), 7.42-7.29 (m, 2H), 7.09 (d, J=9.2 Hz, 2H), 5.37 (s, 2H), 3.02-2.83 (m, 1H), 2.24 (d, J=11.4 Hz, 1H), 2.07 (d, J=11.4 Hz, 2H), 1.97 (d, J=11.4 Hz, 2H), 1.54-1.36 (m, 4H).

### Step 5: Synthesis of trans-4-(5-((4-chlorophenoxy)methyl)-1,3,4-oxadiazol-2-yl)-N-(6-chloroquinolin-2-yl)cyclohexanecarboxamide

To a stirred solution of trans-4-(5-((4-chlorophenoxy)methyl)-1,3,4-oxadiazol-2-yl)cyclohexanecarboxylic acid (50 mg, 0.149 mmol, 1.0 eq.) in DCM:DMF (2:1) (10 mL) was added EDCI.HCl (143 mg, 0.744 mmol, 5.0 eq.) and DMAP (73 mg, 0.595 mmol, 4.0 eq.) at RT and stirred for 15 min. 6-chloroquinolin-2-amine (27 mg, 0.149 mmol, 1.0 eq.) was added and the resulting reaction mixture was stirred at RT for 4 h. Product formation was confirmed by LCMS and TLC. After completion of reaction, the reaction mixture was concentrated under reduced pressure and the residue was diluted with EtOAc (20 mL). The organic layer was washed with water (10 mL) and brine (10 mL), dried over anhydrous Na₂SO₄ and concentrated under reduced pressure. The crude product was crystallized in methanol to obtain trans-4-(5-((4-chlorophenoxy)methyl)-1,3,4-oxadiazol-2-yl)-N-(6-chloroquinolin-2-yl)cyclohexanecarboxamide (Compound 40-14 mg, 18% yield) as a white solid. LCMS 497.5 [M+H]⁺; ¹H NMR (400 MHz, DMSO-d₆) δ 10.85 (s, 1H), 8.43-8.27 (m, 2H), 8.05 (d, J=2.2 Hz, 1H), 7.80 (d, J=9.2 Hz, 1H), 7.71 (dd, J=2.4, 9.0 Hz, 1H), 7.48-7.30 (m, J=9.2 Hz, 2H), 7.14-6.97 (m, J=8.8 Hz, 2H), 5.47-5.31 (m, 2H), 3.01 (d, J=11.8 Hz, 1H), 2.33 (br. s., 1H), 2.15 (d, J=13.2 Hz, 2H), 1.99 (d, J=13.2 Hz, 2H), 1.66-1.40 (m, 4H).

### Example 39

### Synthesis of trans-N-(5-chlorobenzo[d]oxazol-2-yl)-4-(5-((cis-3-(trifluoromethoxy)cyclobutoxy)methyl)-1,3,4-oxadiazol-2-yl)cyclohexanecarboxamide

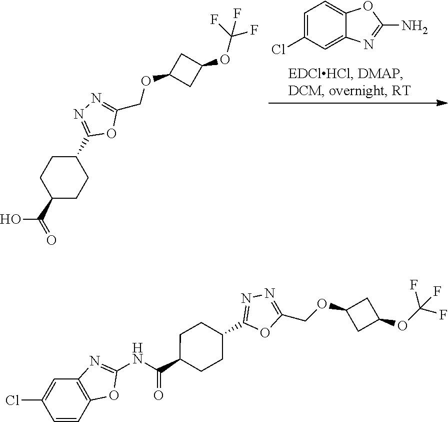 [SMILES: O=C(O)[C@H]1CC[C@H](c2nnc(CO[C@H]3C[C@@H](OC(F)(F)F)C3)o2)CC1 ; Nc1nc2cc(Cl)ccc2o1 ; O=C(Nc1nc2cc(Cl)ccc2o1)[C@H]1CC[C@H](c2nnc(CO[C@H]3C[C@@H](OC(F)(F)F)C3)o2)CC1]

To a stirred solution of trans-4-(5-((cis-3-(trifluoromethoxy)cyclobutoxy)methyl)-1,3,4-oxadiazol-2-yl)cyclohexanecarboxylic acid (50 mg, 0.137 mmol, 1 eq.) in DCM (20 mL) was added EDCI.HCl (120 mg, 0.625 mmol, 4.6 eq.) and DMAP (60 mg, 0.491 mmol, 3.6 eq.) and was stirred at RT for 15 minutes followed by addition of 5-chlorobenzo[d]oxazol-2-amine (23 mg, 0.137 mmol, 1.0 eq.). The resulting reaction mixture was stirred at RT overnight. Product formation was confirmed by LCMS and TLC. After completion of reaction, the reaction mixture was diluted with DCM (20 mL). Organic layer was washed with 1 M HCl solution (10 mL) and brine (10 mL), dried over anhydrous Na₂SO₄ and concentrated under reduced pressure. The crude product was purified by flash chromatography (0-5% methanol in DCM as eluent) to obtained trans-N-(5-chlorobenzo[d]oxazol-2-yl)-4-(5-((cis-3-(trifluoromethoxy)cyclobutoxy)methyl)-1,3,4-oxadiazol-2-yl)cyclohexanecarboxamide (Compound 41-16 mg, 22% yield) as white solid. LCMS 515.5 [M+H]⁺; ¹H NMR (400 MHz, DMSO-d₆) δ 11.78 (br. s., 1H), 7.77-7.57 (m, 2H), 7.31 (d, J=8.8 Hz, 1H), 4.64 (s, 2H), 4.53-4.43 (m, 1H), 3.87-3.76 (m, 1H), 3.02 (br. s., 1H), 2.72 (br. s., 2H), 2.60 (br. s., 1H), 2.15 (d, J=10.1 Hz, 2H), 2.10-1.92 (m, 4H), 1.67-1.42 (m, 4H).

### Example 40

Chiral separation of trans-(S)-N-(4-(5-(6-chloro-3,4-dihydro-2H-benzo[b][1,4]oxazin-2-yl)-1,3,4-oxadiazol-2-yl)cyclohexyl)-2-(4-chloro-3-fluorophenoxy)acetamide and trans-(R)-N-(4-(5-(6-chloro-3,4-dihydro-2B-benzo[b][1,4]oxazin-2-yl)-1,3,4-oxadiazol-2-yl)cyclohexyl)-2-(4-chloro-3-fluorophenoxy)acetamide

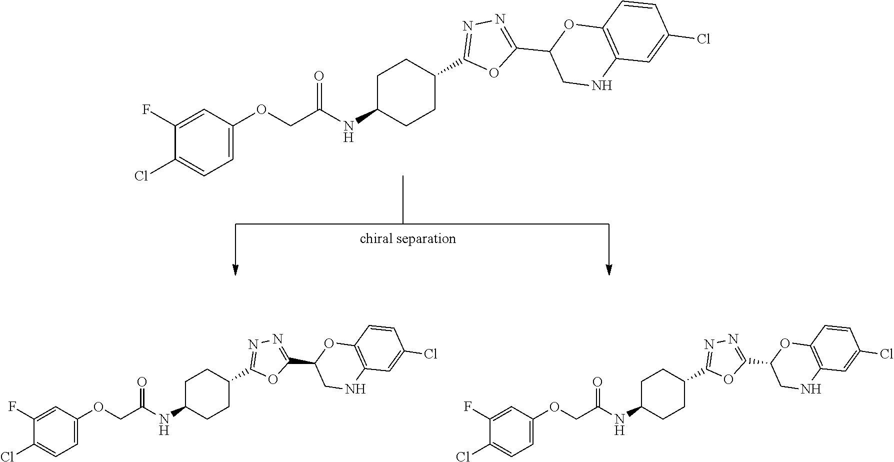 [SMILES: O=C(COc1ccc(Cl)c(F)c1)N[C@H]1CC[C@H](c2nnc(C3CNc4cc(Cl)ccc4O3)o2)CC1 ; O=C(COc1ccc(Cl)c(F)c1)N[C@H]1CC[C@H](c2nnc([C@@H]3CNc4cc(Cl)ccc4O3)o2)CC1 ; O=C(COc1ccc(Cl)c(F)c1)N[C@H]1CC[C@H](c2nnc([C@H]3CNc4cc(Cl)ccc4O3)o2)CC1]

The enantiomers, trans-(S)-N-(4-(5-(6-chloro-3,4-dihydro-2H-benzo[b][1,4]oxazin-2-yl)-1,3,4-oxadiazol-2-yl)cyclohexyl)-2-(4-chloro-3-fluorophenoxy)acetamide (Compound 42-[α]_{D}²⁰=NA; elution time: 14.68 min) and trans-(R)-N-(4-(5-(6-chloro-3,4-dihydro-2H-benzo[b][1,4]oxazin-2-yl)-1,3,4-oxadiazol-2-yl)cyclohexyl)-2-(4-chloro-3-fluorophenoxy)acetamide (Compound 43—[α]_{D}²⁰=NA; elution time: 19.02 min), were separated by chiral SFC (Chiralpak-IG, 250×21 mm, 5μ) using an isocratic program with analytical grade liquid carbon dioxide and HPLC grade methanol (0.2% DEA). LCMS: 521.6 [M+H]⁺.

### Example 41

### Synthesis of trans-2-(4-chloro-3-fluorophenoxy)-N-(4-(4-(4-chlorophenyl)-1H-imidazol-1-yl)cyclohexyl)acetamide

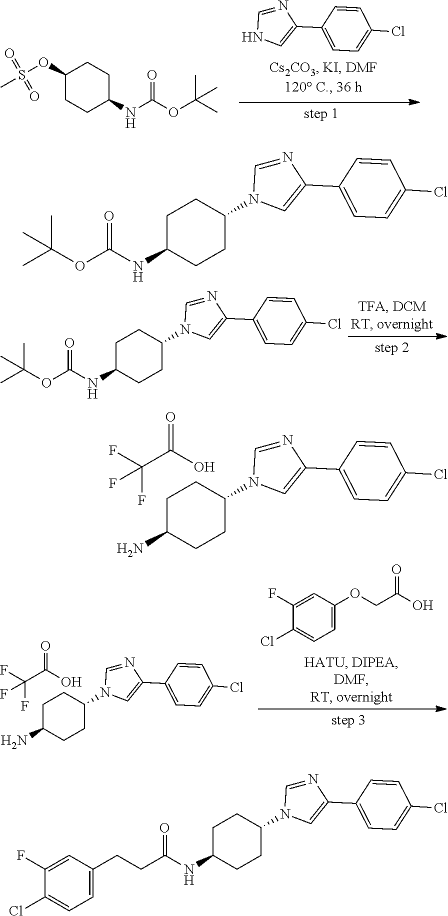

### Step 1: Synthesis of tert-butyl trans-4-(4-(4-chlorophenyl)-1H-imidazol-1-yl)cyclohexylcarbamate

To a stirred mixture of 4-(4-chlorophenyl)-1H-imidazole (180 mg, 1.007 mmol, 1 eq.), cis-4-(tert-butoxycarbonylamino)cyclohexyl methane sulfonate (295 mg, 1.007 mmol, 1 eq.) and Cs₂CO₃ (655 mg, 2.015 mmol, 2.0 eq.) in DMF (5 mL) was added KI (84 mg, 0.682 mmol, 0.5 eq.) after purging with N₂. The resulting reaction mixture was heated at 120° C. for 36 hours. Reaction progress was monitored by LCMS. After completion of reaction, the reaction mixture was diluted with water (20 mL) and extracted with ethyl acetate (2×10 mL). The combined organic layers were washed with cold water (5×5 mL), 1 M HCl (2×5 mL) and brine (10 mL), dried over anhydrous Na₂SO₄ and concentrated under reduced pressure to obtain tert-butyl trans-4-(4-(4-chlorophenyl)-1H-imidazol-1-yl)cyclohexylcarbamate (40 mg, 10% yield) as a brown semi solid. LCMS 376.3 [M+H]⁺.

### Step 2: Synthesis of trans-4-(4-(4-chlorophenyl)-1H-imidazol-1-yl)cyclohexanamine 2,2,2-trifluoroacetate

To a solution of tert-butyl trans-4-(4-(4-chlorophenyl)-1H-imidazol-1-yl)cyclohexylcarbamate (40 mg, 0.106 mmol, 1 eq.) in DCM (2 mL) was added TFA (0.2 mL) under N₂. The reaction mixture was stirred at RT overnight. The product formation was confirmed by LCMS. After completion of reaction the reaction mixture was concentrated under reduced pressure. The crude product was crystallized in diethyl ether to obtain trans-4-(4-(4-chlorophenyl)-1H-imidazol-1-yl)cyclohexanamine 2,2,2-trifluoroacetate (40 mg, 97% yield) as a brown solid. LCMS 276.3 [M+H]⁺.

### Step 3: Synthesis of trans-2-(4-chloro-3-fluorophenoxy)-N-(4-(4-(4-chlorophenyl)-1H-imidazol-1-yl)cyclohexyl)acetamide

To a stirred solution of 4-(1-(4-chlorophenyl)-1H-pyrazol-4-yl)cyclohexanamine 2,2,2-trifluoroacetate (40 mg, 0.102 mmol, 1.0 eq.), 2-(4-chloro-3-fluorophenoxy)acetic acid (21 mg, 0.102 mmol, 1.0 eq.) and HATU (58 mg, 0.153 mmol, 1.5 eq.) in DMF (2 mL) was added DIPFA (0.08 mL, 0.408 mmol, 4.0 eq.). The resulting reaction mixture was stirred at RT overnight. The product formation was confirmed by LCMS. After completion of reaction the reaction mixture was diluted with ice cold water (10 mL) and extracted with ethyl acetate (2×10 mL). The combined organic layers were washed with cold water (4×10 mL) and brine (10 mL), dried over anhydrous Na₂SO₄ and concentrated under reduced pressure. The crude product was purified by flash chromatography (0-5% methanol in DCM as an eluent) to obtain trans-2-(4-chloro-3-fluorophenoxy)-N-(4-(4-(4-chlorophenyl)-1H-imidazol-1-yl)cyclohexyl)acetamide (Compound 44—5 mg, 23% yield) as a white solid. LCMS 462.4 [M+H]⁺; ¹H NMR (400 MHz, DMSO-d₆) δ 8.07 (d, J=7.5 Hz, 1H), 7.82 (s, 1H), 7.80-7.67 (m, 2H), 7.51 (t, J=8.8 Hz, 1H), 7.39 (d, J=8.8 Hz, 2H), 7.08 (dd, J=2.6, 11.4 Hz, 1H), 6.86 (d, J=9.2 Hz, 1H), 4.52 (s, 2H), 4.08 (br. s., 1H), 3.74 (br. s., 1H), 2.04 (d, J=10.5 Hz, 2H), 1.90-1.76 (m, 4H), 1.61-1.36 (m, 2H).

### Example 42

### Synthesis of trans-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide

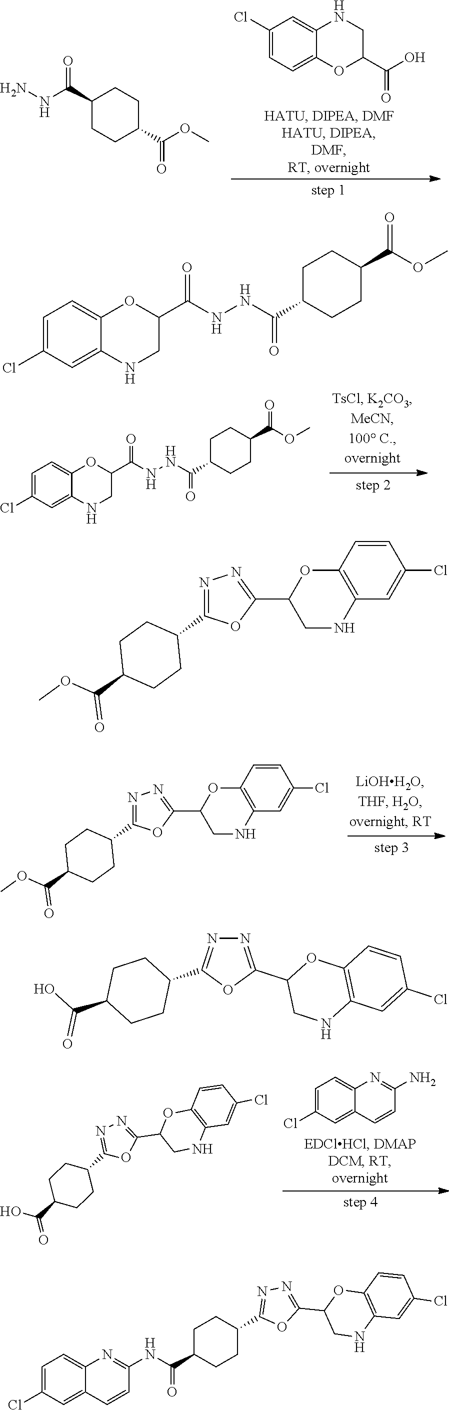

### Step 1: Synthesis of trans-methyl 4-(2-(6-chloro-3,4-dihydro-2H-benzo[b][1,4]oxazine-2-carbonyl)hydrazinecarbonyl)cyclohexanecarboxylate

To a solution of trans-methyl 4-(hydrazinecarbonyl)cyclohexanecarboxylate (250 mg, 1.25 mmol, 1.0 eq.), 6-chloro-3,4-dihydro-2H-benzo[b][1,4]oxazine-2-carboxylic acid (319 mg, 1.5 mmol, 1.2 eq.) and HATU (950 mg, 2.5 mmol, 2.0 eq.) in DMF (3.0 mL) was added DIPEA (0.4 ml, 2.5 mmol, 2.0 eq.). The resulting reaction mixture was allowed to stir at RT overnight. The product formation was confirmed by LCMS. After completion of reaction, the reaction mixture was diluted with cold water (50 mL). The resulting solid was filtered off, washed with water and dried under vacuum. The crude product was crystallized in methanol to obtain trans-methyl 4-(2-(6-chloro-3,4-dihydro-2H-benzo[b][1,4]oxazine-2-carbonyl)hydrazinecarbonyl)cyclohexanecarboxylate (170 mg, 34% yield) as an off-white solid. LCMS 396.3 [M+H]⁺; ¹H NMR (400 MHz, DMSO-d₆) δ 9.94 (s, 1H) 9.76 (s, 1H) 6.76 (d, J=8.33 Hz, 1H) 6.60 (d, J=2.63 Hz, 1H) 6.51 (dd, J=8.55, 2.41 Hz, 1H) 6.19 (br. s., 1H) 4.61 (dd, J=6.80, 2.85 Hz, 1H) 3.59 (s, 3H) 3.44 (d, J=12.28 Hz, 1H) 3.25 (dd, J=10.52, 7.02 Hz, 1H) 2.22-2.38 (m, 1H) 2.12-2.22 (m, 1H) 1.93 (d, J=12.28 Hz, 2H) 1.77 (d, J=10.52 Hz, 2H) 1.21-1.49 (m, 4H).

### Step 2: Synthesis of trans-methyl 4-(5-(6-chloro-3,4-dihydro-2H-benzo[b][1,4]oxazin-2-yl)-1,3,4-oxadiazol-2-yl)cyclohexanecarboxylate

To a stirred mixture of trans-methyl 4-(2-(6-chloro-3,4-dihydro-2H-benzo[b][1,4]oxazine-2-carbonyl)hydrazinecarbonyl)cyclohexanecarboxylate (250 mg, 0.632 mmol, 1.0 eq.), K₂CO₃ (262 mg, 1.898 mmol, 3.0 eq.) and molecular sieves (250 mg) in acetonitrile (40 mL) was added 4-toluenesulfonyl chloride (300 mg, 1.58 mmol, 2.5 eq.). The resulting reaction mixture was stirred at 100° C. overnight. Reaction was monitored by LCMS and TLC. After completion of reaction the reaction mixture was filtered and filtrate was diluted with EtOAc (25 mL). The organic layer was washed with water (20 mL) and brine (20 mL), dried over anhydrous Na₂SO₄ and concentrated under reduced pressure to obtain trans-methyl 4-(5-(6-chloro-3,4-dihydro-2H-benzo[b][1,4]oxazin-2-yl)-1,3,4-oxadiazol-2-yl)cyclohexanecarboxylate (250 mg, quant. yield) as a brown solid. LCMS 378.3 [M+H]⁺; ¹H NMR (400 MHz, DMSO-d₆) δ 6.77 (d, J=8.77 Hz, 1H) 6.64 (d, J=2.63 Hz, 1H) 6.51 (dd, J=8.33, 2.63 Hz, 1H) 6.36 (br. s., 1H) 5.49 (dd, J=6.14, 3.07 Hz, 1H) 3.46-3.73 (m, 5H) 2.92-3.06 (m, 1H) 2.25-2.41 (m, 1H) 2.01-2.17 (m, 2H) 1.98 (d, J=10.09 Hz, 2H) 1.32-1.61 (m, 4H).

### Step 3: Synthesis of trans-4-(5-(6-chloro-3,4-dihydro-2H-benzo[b][1,4]oxazin-2-yl)-1,3,4-oxadiazol-2-yl)cyclohexanecarboxylic acid

To a stirred solution of trans-methyl 4-(5-(6-chloro-3,4-dihydro-2H-benzo[b][1,4]oxazin-2-yl)-1,3,4-oxadiazol-2-yl)cyclohexanecarboxylate (250 mg, 0.663 mmol, 1.0 eq.) in THE (5 mL) was added LiOH.H₂O (41 mg, 0.994 mmol, 1.5 eq.) and H₂O (5 ml). The resulting reaction mixture was stirred at RT overnight. The product formation was confirmed by LCMS. After completion of reaction the reaction mixture was concentrated under reduced pressure. The resulting residue was dissolved in water (10 mL), acidified with 1 M HCl (pH 4-5). The resulting solid was filtered off and dried under vacuum to obtain trans-4-(5-(6-chloro-3,4-dihydro-2H-benzo[b][1,4]oxazin-2-yl)-1,3,4-oxadiazol-2-yl)cyclohexanecarboxylic acid (200 mg, 83% yield) as a brown solid. LCMS 364.3 [M+H]⁺.

### Step 4: Synthesis of trans-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide

To a stirred solution of trans-4-(5-(6-chloro-3,4-dihydro-2H-benzo[b][1,4]oxazin-2-yl)-1,3,4-oxadiazol-2-yl)cyclohexanecarboxylic acid (180 mg, 0.495 mmols, 1.0 eq.), DMAP (72 mg, 0.595 mmol, 1.2 eq.) and EDC.HCl (142 mg, 0.742 mmol, 1.5 eq.) in DCM (10 mL) was added 6-chloroquinolin-2-amine (105 mg, 0.595 mmol, 1.2 eq.) at RT. The resulting reaction mixture was stirred at RT overnight. Product formation was confirmed by LCMS. After completion of reaction the reaction mixture was concentrated under reduced pressure. The resulting residue was diluted with 1 M HCl (10 mL) and extracted with ethyl acetate (25 mL×2). The combined organic layers were washed with brine, dried over Na₂SO₄ and concentrated under reduced pressure. The crude product was purified by flash chromatography (0-5% MeOH in DCM as an eluent) and then crystallized in methanol to obtain trans-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide (Compound 45-90 mg, 34% yield) as an off white solid. LCMS 524.5 [M+H]⁺; ¹H NMR (400 MHz, DMSO-d₆) δ 10.85 (s, 1H) 8.24-8.41 (m, 2H) 8.05 (d, J=2.19 Hz, 1H) 7.80 (d, J=9.21 Hz, 1H) 7.68-7.75 (m, 1H) 6.79 (d, J=8.33 Hz, 1H) 6.65 (d, J=2.19 Hz, 1H) 6.52 (dd, J=8.55, 2.41 Hz, 1H) 6.37 (br. s., 1H) 5.51 (dd, J=6.14, 3.07 Hz, 1H) 3.61-3.76 (m, 1H) 3.54-3.61 (m, 1H) 3.01 (t, J=11.18 Hz, 1H) 2.65 (d, J=16.66 Hz, 1H) 2.14 (d, J=10.96 Hz, 2H) 1.99 (d, J=11.84 Hz, 2H) 1.42-1.72 (m, 4H).

### Example 43

### Synthesis of trans-N-(6-chloroquinolin-2-yl)-4-(5-(6-chloroquinolin-2-yl)-1,3,4-oxadiazol-2-yl)cyclohexanecarboxamide

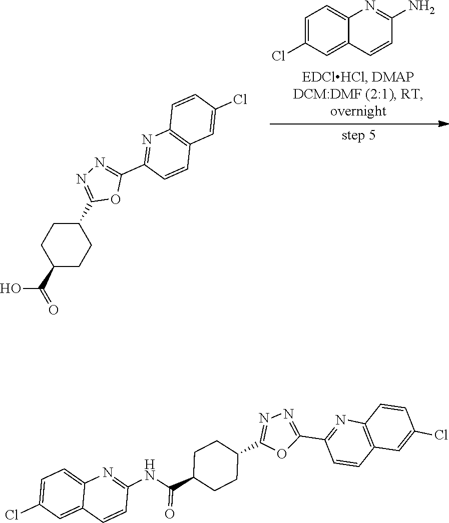 [SMILES: Nc1ccc2cc(Cl)ccc2n1 ; O=C(O)[C@H]1CC[C@H](c2nnc(-c3ccc4cc(Cl)ccc4n3)o2)CC1 ; O=C(Nc1ccc2cc(Cl)ccc2n1)[C@H]1CC[C@H](c2nnc(-c3ccc4cc(Cl)ccc4n3)o2)CC1]

To a stirred solution of trans-4-(5-(6-chloroquinolin-2-yl)-1,3,4-oxadiazol-2-yl)cyclohexanecarboxylic acid (80 mg, 0.224 mmol, 1.0 eq.), DMAP (32 mg, 0.268 mmol, 1.2 eq.) and EDCl.HCl (64 mg, 0.336 mmol, 1.5 eq.) in DCM (20 mL) was added 6-chloroquinolin-2-amine (47 mg, 0.268 mmol, 1.2 eq.) at RT. The resulting reaction mixture was stirred at RT overnight. Product formation was confirmed by LCMS. After completion of reaction the reaction mixture was concentrated under reduced pressure. The resulting residue was diluted with 1 M HCl (10 mL) and extracted with ethyl acetate (25 mL×2). The combined organic layers were washed with brine, dried over Na₂SO₄ and concentrated under reduced pressure. The crude product was purified by flash chromatography (0-5% MeOH in DCM as an eluent) and then crystallized in methanol to obtain trans-N-(6-chloroquinolin-2-yl)-4-(5-(6-chloroquinolin-2-yl)-1,3,4-oxadiazol-2-yl)cyclohexanecarboxamide (Compound 46-16 mg, 13% yield) as a white solid. LCMS 518.5 [M+H]⁺; ¹H NMR (400 MHz, DMSO-d₆) δ10.88 (br. s., 1H) 8.61 (d, J=8.33 Hz, 1H) 8.28-8.57 (m, 4H) 8.22 (d, J=9.21 Hz, 1H) 8.06 (br. s., 1H) 7.91 (d, J=8.77 Hz, 1H) 7.82 (d, J=9.21 Hz, 1H) 7.73 (br. s., 1H) 3.18 (br. s., 1H) 2.67 (br. s., 1H) 2.30 (d, J=18.42 Hz, 2H) 2.05 (br. s., 2H) 1.67-1.52 (br. s., 4H).

### Example 44

### Synthesis of trans-4-(5-(4-chlorophenyl)-1,3,4-oxadiazol-2-yl)-N-((6-chloroquinolin-2-yl)methyl)cyclohexanecarboxamide

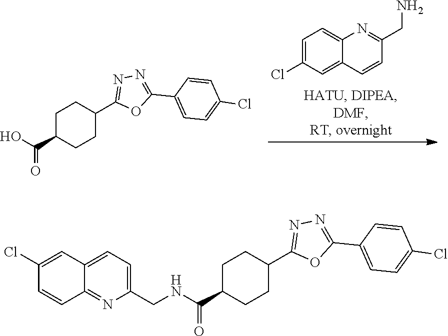

To a stirred solution of trans-4-(5-(4-chlorophenyl)-1,3,4-oxadiazol-2-yl)cyclohexanecarboxylic acid (20 mg, 0.065 mmol, 1.0 eq.) and (6-chloroquinolin-2-yl)methanamine (25.09 mg, 0.130 mmol, 2.0 eq.) in DMF (1.0 mL) was added HATU (49.4 mg, 0.130 mmol, 2.0 eq.) and DIPEA (0.04 mL, 0.195 mmol, 3.0 eq.). The resulting reaction mixture was allowed to stir overnight at room temperature. The product formation was confirmed by LCMS. After completion of reaction the reaction mixture was diluted with water (2 mL). The resulting solid was filtered off, washed with diethyl ether and dried under vacuum to obtain trans-4-(5-(4-chlorophenyl)-1,3,4-oxadiazol-2-yl)-N-((6-chloroquinolin-2-yl)methyl)cyclohexanecarboxamide (Compound 47-4.0 mg, 13% yield) as a white solid. LCMS 481.5 [M+H]⁺; ¹H NMR (400 MHz, DMSO-d₆) δ 8.56 (br. s., 1H) 8.33 (d, J=8.33 Hz, 1H) 8.10 (d, J=2.19 Hz, 1H) 7.94-8.05 (m, 3H) 7.73-7.80 (m, 1H) 7.67 (d, J=8.77 Hz, 2H) 7.48 (d, J=8.77 Hz, 1H) 4.54 (d, J=6.14 Hz, 2H) 2.33 (br. s., 2H) 2.21 (br. s., 2H) 1.96 (br. s., 2H) 1.61 (t, J=10.30 Hz, 3H).

### Example 45

### Synthesis of trans-N-(6-chloroquinolin-2-yl)-4-(5-(cis-3-(trifluoromethoxy)cyclobutyl)-1,3,4-oxadiazol-2-yl)cyclohexanecarboxamide

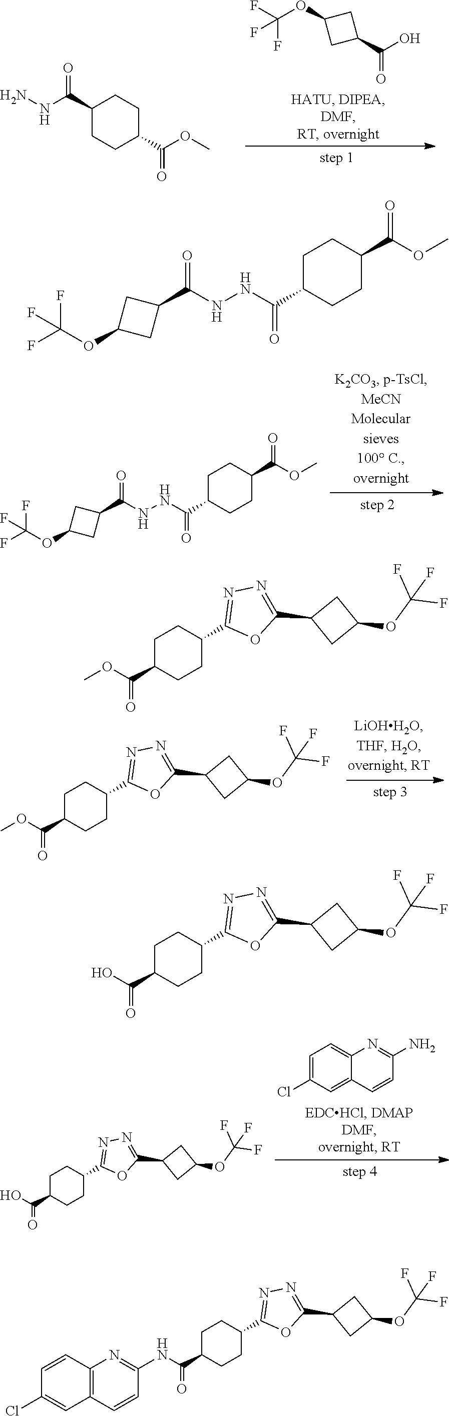

### Step 1: Synthesis of trans-methyl 4-(2-(cis-3-(trifluoromethoxy)cyclobutanecarbonyl)hydrazinecarbonyl)cyclohexanecarboxylate

To a stirred solution of trans-methyl 4-(hydrazinecarbonyl)cyclohexanecarboxylate (0.100 g, 0.500 mmol, 1.0 eq.) and cis-3-(trifluoromethoxy)cyclobutanecarboxylic acid (0.092 g, 0.500 mmol, 1.0 eq.) in DMF (2 mL) was added HATU (0.285 g, 0.750 mmol, 1.5 eq.) DIPEA (0.25 mL, 1.50 mmol, 3.0 eq.) and the resulting reaction mixture was stirred at RT overnight. The product formation was confirmed by LCMS. After completion of reaction the reaction mixture was poured into water (5 ml). The resulting solid was filtered off and dried under vacuum to obtain trans-methyl 4-(2-(cis-3-(trifluoromethoxy)cyclobutanecarbonyl)hydrazinecarbonyl)cyclohexanecarboxylate (0.080 g, 43% yield) as a white solid. ¹H NMR (400 MHz, DMSO-d₆) δ 9.82 (s, 1H) 9.71 (s, 1H) 4.73-4.85 (m, 1H) 3.59 (s, 3H) 2.59-2.75 (m, 2H) 2.19-2.38 (m, 3H) 2.15 (br. s., 1H) 1.93 (d, J=10.09 Hz, 2H) 1.76 (d, J=12.28 Hz, 2H) 1.20-1.49 (m, 5H).

### Step 2: Synthesis of trans-methyl 4-(5-(cis-3-(trifluoromethoxy)cyclobutyl)-1,3,4-oxadiazol-2-yl)cyclohexanecarboxylate

To a stirred solution of trans-methyl 4-(2-(cis-3-(trifluoromethoxy)cyclobutanecarbonyl)hydrazinecarbonyl)cyclohexanecarboxylate (0.030 g, 0.081 mmol, 1.0 eq.), K₂CO₃ (0.056 g, 0.409 mmol, 5.0 eq.) and molecular sieves (250 mg) in acetonitrile (2 mL) was added 4-toluenesulfonyl chloride (0.033 g, 0.204 mmol, 2.5 eq.). The resulting reaction mixture heated at 100° C. for overnight. The product formation was confirmed by LCMS and TLC. After completion of reaction the reaction mixture was filtered and filtrate was diluted with EtOAc (15 mL). The organic layer was washed with water (20 mL) and brine (20 mL), dried over anhydrous Na₂SO₄ and concentrated under reduced pressure to obtain trans-methyl 4-(5-(cis-3-(trifluoromethoxy)cyclobutyl)-1,3,4-oxadiazol-2-yl)cyclohexanecarboxylate (0.030 g quant. yield) as an off white solid. LCMS 349.3 [M+H]⁺.

### Step 3: Synthesis of trans-4-(5-(cis-3-(trifluoromethoxy)cyclobutyl)-1,3,4-oxadiazol-2-yl)cyclohexanecarboxylic acid

To a stirred solution of trans-methyl 4-(5-(cis-3-(trifluoromethoxy)cyclobutyl)-1,3,4-oxadiazol-2-yl)cyclohexanecarboxylate (0.030 g, 0.129 mmol, 1.0 eq.) in THE (1 mL) was added LiOH.H₂O (0.008 g, 0.193 mmol, 1.5 eq.) and H₂O (1 ml) and the resulting reaction mixture was stirred at RT for 3 h. The product formation was confirmed by LCMS. After completion of reaction, the reaction mixture was concentrated under reduced pressure and the resulting residue was dissolved in water (1 mL), acidified with 1 M HCl, extracted with ethyl acetate (2×3 mL), dried over anhydrous Na₂SO₄ and concentrated under reduced pressure to obtain trans-4-(5-(cis-3-(trifluoromethoxy)cyclobutyl)-1,3,4-oxadiazol-2-yl)cyclohexanecarboxylic acid (0.020 g, 71% yield) as a white solid. LCMS 335.3 [M+H]⁺.

### Step 4: Synthesis of trans-N-(6-chloroquinolin-2-yl)-4-(5-(cis-3-(trifluoromethoxy)cyclobutyl)-1,3,4-oxadiazol-2-yl)cyclohexanecarboxamide

To a stirred solution of trans-4-(5-(cis-3-(trifluoromethoxy)cyclobutyl)-1,3,4-oxadiazol-2-yl)cyclohexanecarboxylic acid (0.020 g, 0.059 mmol, 1 eq.), 6-chloroquinolin-2-amine (0.005 g, 0.029 mmol, 0.5 eq.) in DMF (0.5 mL) was added EDC.HCl (0.017 g, 0.089 mmol, 1.5 eq.) followed by the addition of DMAP (0.010 g, 0.089 mmol, 1.5 eq.) at RT. The resulting reaction mixture was stirred at RT overnight. The product formation was confirmed by LCMS. After completion of reaction the reaction mixture was diluted with water (5 ml) and extracted with ethyl acetate (2×6 mL). The combined organic layers were washed with water (3 mL×2), dried over anhydrous Na₂SO₄ and concentrated under reduced pressure. The crude product was purified by flash chromatography (0-5% MeOH/DCM as an eluent) to obtain trans-N-(6-chloroquinolin-2-yl)-4-(5-(cis-3-(trifluoromethoxy)cyclobutyl)-1,3,4-oxadiazol-2-yl)cyclohexanecarboxamide (Compound 48-0.003 g) as a white solid. LCMS 495.5[M+H]⁺; ¹H NMR (400 MHz, DMSO-d₆) δ 10.84 (s, 1H) 8.25-8.42 (m, 2H) 8.05 (d, J=2.19 Hz, 1H) 7.81 (d, J=9.21 Hz, 1H) 7.71 (dd, J=8.99, 2.41 Hz, 1H) 4.81-4.99 (m, 1H) 3.41 (d, J=8.33 Hz, 2H) 2.95 (br. s., 1H) 2.84 (d, J=10.09 Hz, 2H) 2.59-2.71 (m, 2H) 2.14 (d, J=11.40 Hz, 2H) 1.99 (d, J=10.96 Hz, 2H) 1.39-1.68 (m, 4H).

### Example 46

### Synthesis of 1-(5-(4-chlorophenyl)-1,3,4-oxadiazol-2-yl)-N-(6-chloroquinolin-2-yl)piperidine-4-carboxamide

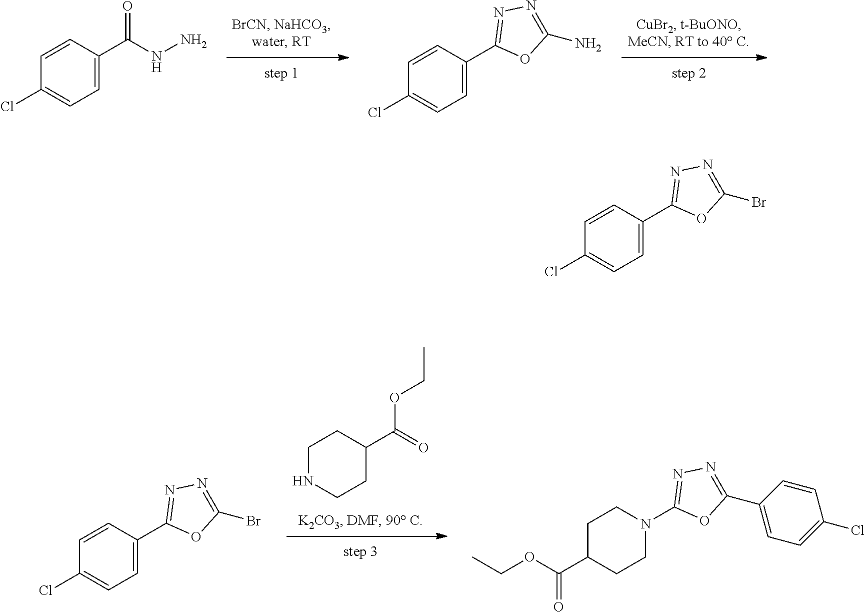 [SMILES: NNC(=O)c1ccc(Cl)cc1 ; Nc1nnc(-c2ccc(Cl)cc2)o1 ; Clc1ccc(-c2nnc(Br)o2)cc1 ; Clc1ccc(-c2nnc(Br)o2)cc1 ; CCOC(=O)C1CCNCC1 ; CCOC(=O)C1CCN(c2nnc(-c3ccc(Cl)cc3)o2)CC1]

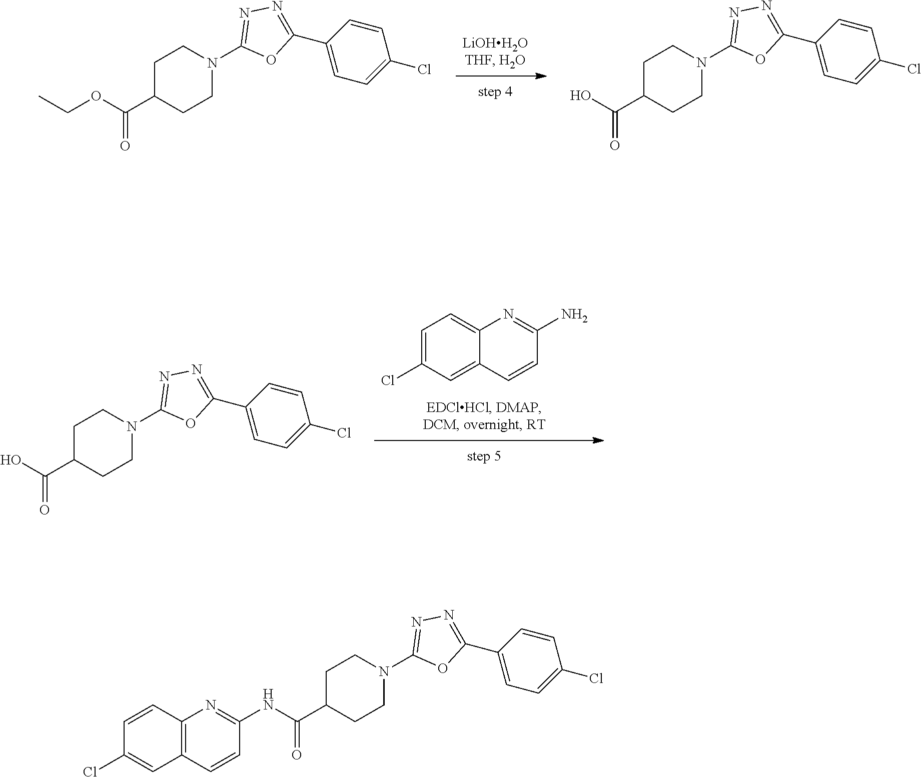 [SMILES: CCOC(=O)C1CCN(c2nnc(-c3ccc(Cl)cc3)o2)CC1 ; O=C(O)C1CCN(c2nnc(-c3ccc(Cl)cc3)o2)CC1 ; O=C(O)C1CCN(c2nnc(-c3ccc(Cl)cc3)o2)CC1 ; Nc1ccc2cc(Cl)ccc2n1 ; O=C(Nc1ccc2cc(Cl)ccc2n1)C1CCN(c2nnc(-c3ccc(Cl)cc3)o2)CC1]

### Step 1: Synthesis of 5-(4-chlorophenyl)-1,3,4-oxadiazol-2-amine

To a stirred solution of 4-chlorobenzohydrazide (500 mg, 2.92 mmol, 1.0 eq.) in 1-4 dioxane (5 mL), was added NaHCO₃ (360 mg, 4.38 mmol, 1.5 eq.) in water (3 ml) followed by the addition of cyanogen bromide (300 mg, 2.92 mmol, 1.0 eq.). The resulting reaction mixture was stirred at RT overnight. The product formation was confirmed by LCMS. After completion of reaction the reaction mixture diluted with EtOAc (100 mL). The organic layer was washed with water (50 mL) and brine (50 mL), dried over anhydrous Na₂SO₄ and concentrated under reduced pressure. The crude product was purified by flash chromatography (0-30% ethyl acetate in hexane as an eluent) to obtain 5-(4-chlorophenyl)-1,3,4-oxadiazol-2-amine (300 mg, 52% yield) as a brown solid. LCMS 196.1 [M+H]⁺.

### Step 2: Synthesis of 2-bromo-5-(4-chlorophenyl)-1,3,4-oxadiazole

To a stirred mixture of CuBr₂ (250 mg, 1.27 mmol, 1.0 eq.) in MeCN (3 ml) under nitrogen atmosphere was added tert-butyl nitrite (289 mg, 2.806 mmol, 2.2 eq.) dropwise and stirred for 15 min at RT. 5-(4-chlorophenyl)-1,3,4-oxadiazol-2-amine (250 mg, 1.27 mmol, 1.0 eq.) in MeCN (3 ml) was added and the resulting reaction mixture was stirred at RT for 2 h. After completion of reaction the reaction mixture was diluted with EtOAc (100 mL). The organic layer was washed with water (50 mL) and brine (50 mL), dried over anhydrous Na₂SO₄ and concentrated under reduced pressure. The crude product was purified by flash chromatography (0-30% ethyl acetate in hexane as an eluent) to obtain 2-bromo-5-(4-chlorophenyl)-1,3,4-oxadiazole (170 mg, 52% yield) as a brown solid. LCMS 259.1 [M+H]⁺.

### Step 3: Synthesis of ethyl 1-(5-(4-chlorophenyl)-1,3,4-oxadiazol-2-yl)piperidine-4-carboxylate

To a stirred mixture of 2-bromo-5-(4-chlorophenyl)-1,3,4-oxadiazole (60 mg, 0.232 mmol, 1.0 eq.), ethyl piperidine-4-carboxylate (36 mg, 0.232 mmol, 1.0 eq.) in DMF (1 mL) was added potassium carbonate (64 mg, 0.465 mmol, 2.0 eq.). The resulting reaction mixture was stirred at 90° C. for 2 h. The product formation was confirmed by LCMS. After completion of reaction the reaction mixture was diluted with EtOAc (100 mL). The organic layer was washed with water (50 mL) and brine (50 mL), dried over anhydrous Na₂SO₄ and concentrated under reduced pressure. The crude product was purified by flash chromatography (0-30% ethyl acetate in hexane as an eluent) to obtain ethyl 1-(5-(4-chlorophenyl)-1,3,4-oxadiazol-2-yl)piperidine-4-carboxylate (40 mg, 51% yield) as a brown solid. LCMS 336.3 [M+H]⁺.

### Step 4: Synthesis of 1-(5-(4-chlorophenyl)-1,3,4-oxadiazol-2-yl)piperidine-4-carboxylic acid

To a stirred solution of ethyl 1-(5-(4-chlorophenyl)-1,3,4-oxadiazol-2-yl)piperidine-4-carboxylate (40 mg, 0.119 mmol, 1 eq.) in THE (5 mL) was added LiOH.H₂O (15 mg, 0.357 mmol, 3.0 eq.) in water (5 mL) and the resulting reaction mixture was stirred at RT for 1 h. The product formation was confirmed by LCMS. After completion of reaction the reaction mixture was concentrated under reduced pressure. The resulting residue was dissolved in water (10 mL) and washed with EtOAc (10 mL×2). The aqueous layer was separated and acidified with 1 M HCl at pH-3. The resulting solid was filtered off and dried under vacuum to obtain 1-(5-(4-chlorophenyl)-1,3,4-oxadiazol-2-yl)piperidine-4-carboxylic acid (20 mg, 55% yield) as an off white solid. LCMS 308.2 [M+H]⁺.

### Step 5: Synthesis of 1-(5-(4-chlorophenyl)-1,3,4-oxadiazol-2-yl)-N-(6-chloroquinolin-2-yl)piperidine-4-carboxamide

To a stirred solution of 1-(5-(4-chlorophenyl)-1,3,4-oxadiazol-2-yl)piperidine-4-carboxylic acid (25 mg, 0.081 mmol, 1.0 eq.), 6-chloroquinolin-2-amine (15 mg, 0.081 mmol, 1 eq.) and EDC.HCl (60 mg, 0.325 mmol, 4.0 eq.) in DCM (5 mL) was added DMAP (40 mg, 0.325 mmol, 4.0 eq.). The resulting reaction mixture was stirred at RT overnight. The product formation was confirmed by LCMS. After completion of reaction the reaction mixture diluted with DCM (30 mL). The organic layer washed with water (5 mL) and brine (5 mL), dried over anhydrous Na₂SO₄ and concentrated under reduced pressure. The crude product was purified by reverse phase HPLC to obtain 1-(5-(4-chlorophenyl)-1,3,4-oxadiazol-2-yl)-N-(6-chloroquinolin-2-yl)piperidine-4-carboxamide (Compound 49-3 mg, 8% yield) as a white solid. LCMS 468.5 [M+H]⁺; ¹H NMR (400 MHz, DMSO-d₆) δ 10.96 (s, 1H), 8.34 (s, 2H), 8.05 (d, J=2.2 Hz, 1H), 7.99-7.85 (m, J=8.3 Hz, 2H), 7.82 (d, J=9.2 Hz, 1H), 7.76-7.68 (m, 1H), 7.67-7.49 (m, J=8.8 Hz, 2H), 4.05 (d, J=13.6 Hz, 2H), 3.15 (d, J=10.5 Hz, 2H), 2.85 (br. s., 1H), 1.97 (d, J=11.8 Hz, 2H), 1.74 (d, J=9.6 Hz, 2H).

### Example 47

### Synthesis of N-(6-chloroquinolin-2-yl)-1-(5-(cis-3-(trifluoromethoxy)cyclobutyl)-1,3,4-oxadiazol-2-yl)piperidine-4-carboxamide

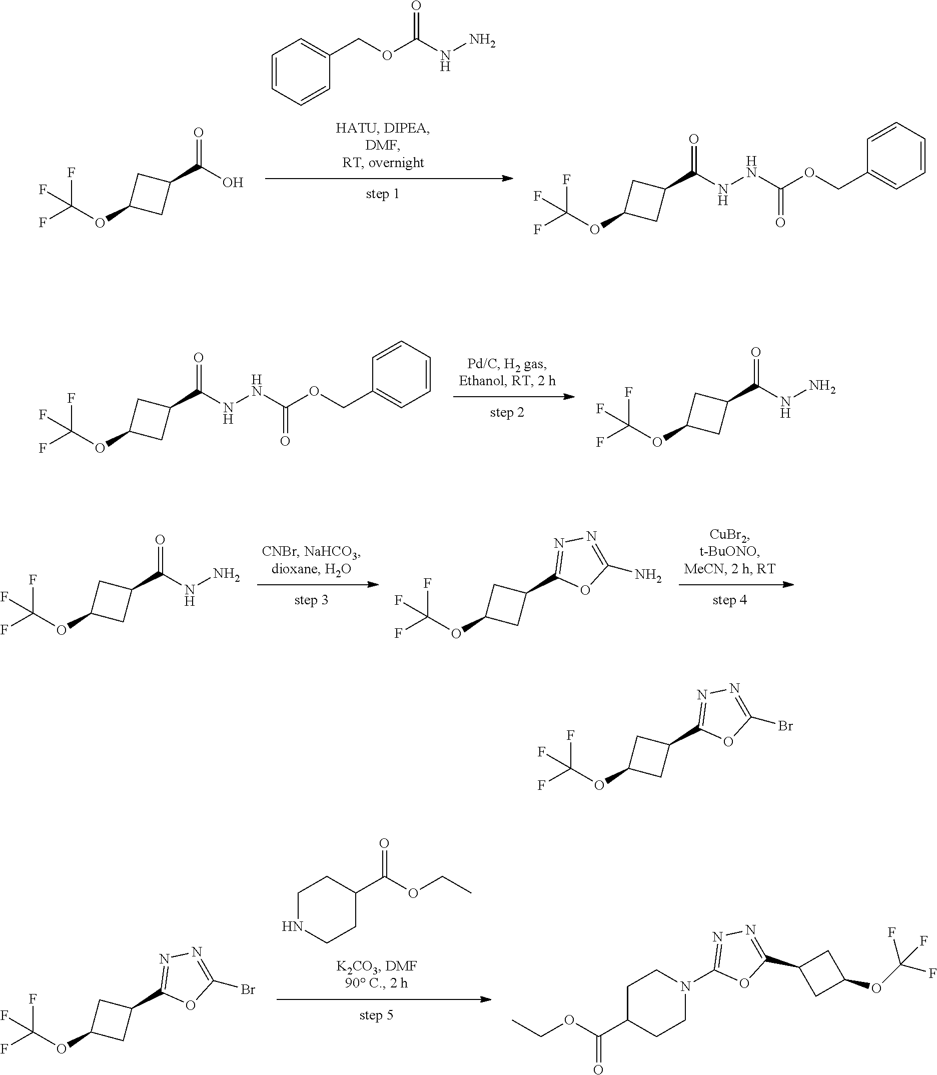

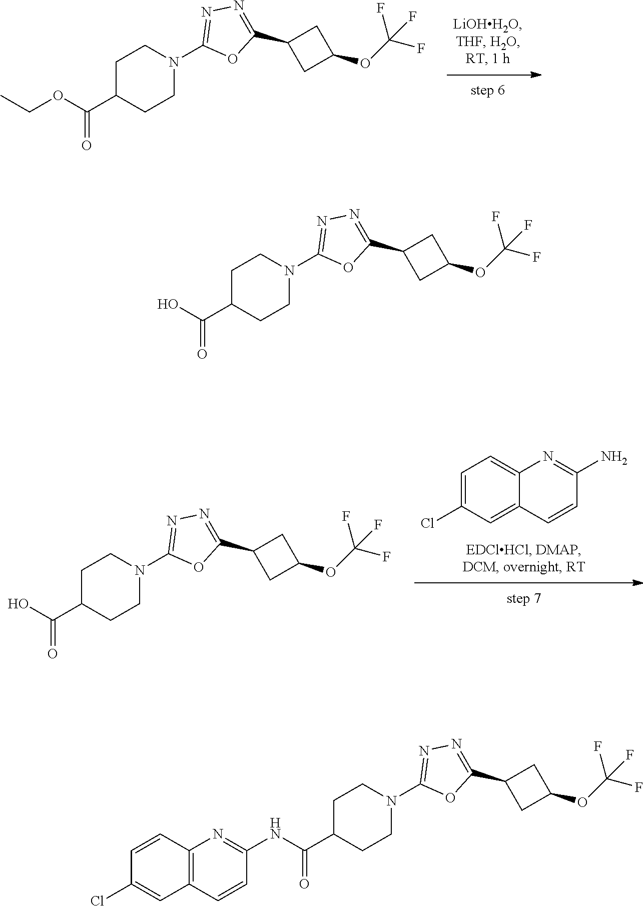 [SMILES: CCOC(=O)C1CCN(c2nnc([C@H]3C[C@@H](OC(F)(F)F)C3)o2)CC1 ; O=C(O)C1CCN(c2nnc([C@H]3C[C@@H](OC(F)(F)F)C3)o2)CC1 ; O=C(O)C1CCN(c2nnc([C@H]3C[C@@H](OC(F)(F)F)C3)o2)CC1 ; Nc1ccc2cc(Cl)ccc2n1 ; O=C(Nc1ccc2cc(Cl)ccc2n1)C1CCN(c2nnc([C@H]3C[C@@H](OC(F)(F)F)C3)o2)CC1]

### Step 1: Synthesis of benzyl 2-(cis-3-(trifluoromethoxy)cyclobutanecarbonyl)hydrazinecarboxylate

To a stirred solution of cis-3-(trifluoromethoxy)cyclobutanecarboxylic acid (300 mg, 1.163 mmol, 1.0 eq.), benzyl hydrazinecarboxylate (270 mg, 1.630 mmol, 1 eq.) and HATU (743 mg, 0.178 mmol, 1.5 eq.) in DMF (5 mL) was added DIPEA (0.58 mL, 3.260 mmol, 2.0 eq.). The resulting reaction mixture was stirred at RT overnight. The product formation was confirmed by LCMS. After completion of reaction the reaction mixture was poured into ice cold water (100 ml). The resulting solid was filtered off and dried under vacuum. The crude product was crystallized in diethyl ether to obtain benzyl 2-(cis-3-(trifluoromethoxy)cyclobutanecarbonyl)hydrazinecarboxylate (170 mg, 34% yield) as a white solid. LCMS 333.3 [M+H]⁺; ¹H NMR (400 MHz, DMSO-d₆) δ 7.36 (s, 5H), 7.26 (s, 1H), 7.21 (br. s., 1H), 6.69 (br. s., 1H), 5.18 (s, 2H), 4.60-4.51 (m, 1H), 2.57 (br. s., 4H).

### Step 2: Synthesis of cis-3-(trifluoromethoxy)cyclobutanecarbohydrazide

To a stirred solution of benzyl 2-(cis-3-(trifluoromethoxy)cyclobutanecarbonyl)hydrazinecarboxylate (230 mg, 0.69 mmol, 1.0 eq.) in ethanol (5 mL) was added Pd/C (115 mg) under nitrogen atmosphere and purged with H₂ gas at RT for 2 h. The product formation was confirmed by LCMS and TLC. After completion of reaction the reaction mixture was filtered through Celite®. The filtrate was concentrated under reduced pressure to obtain cis-3-(trifluoromethoxy)cyclobutanecarbohydrazide (80 mg, 59% yield) as a yellow oil. LCMS 199.2 [M+H]⁺; ¹H NMR (400 MHz, DMSO-d₆) δ 9.10 (br. s., 1H), 4.76 (td, J=7.6, 15.0 Hz, 1H), 4.22 (d, J=3.9 Hz, 2H), 2.63-2.52 (m, 1H), 2.47-2.36 (m, 2H), 2.36-2.20 (m, 2H).

### Step 3: Synthesis of 5-(cis-3-(trifluoromethoxy)cyclobutyl)-1,3,4-oxadiazol-2-amine

To a stirred solution of cis-3-(trifluoromethoxy)cyclobutanecarbohydrazide ((80 mg, 0.402 mmol, 1.0 eq.) in 1-4 dioxane (3 mL), was added NaHCO₃ (50 mg, 0.603 mmol, 1.5 eq.) in water (1 mL). Cyanogen bromide (42 mg, 0.402 mmol, 1.0 eq.) was added and the resulting reaction mixture was stirred at RT overnight. The product formation was confirmed by LCMS. After completion of reaction the reaction mixture was diluted with EtOAc (50 mL). The organic layer was washed with water (20 mL) and brine (20 mL), dried over anhydrous Na₂SO₄ and concentrated under reduced pressure to obtain 5-(cis-3-(trifluoromethoxy)cyclobutyl)-1,3,4-oxadiazol-2-amine (60 mg, 66% yield) as a yellow oil. LCMS 224.2 [M+H]⁺.

### Step 4: Synthesis of 2-bromo-5-(cis-3-(trifluoromethoxy)cyclobutyl)-1,3,4-oxadiazole

To a stirred solution of CuBr₂ (131 mg, 0.58 mmol, 1.0 eq.) in MeCN (5 mL) under nitrogen atmosphere was added t-BuONa (60 mg, 0.580 mmol, 2.2 eq.) dropwise and stirred for 15 min at RT. 5-(cis-3-(trifluoromethoxy)cyclobutyl)-1,3,4-oxadiazol-2-amine (60 mg, 1.27 mmol, 1.0 eq.) in MeCN (3 mL) was added and the resulting reaction mixture was stirred at RT for 2 h. After completion of reaction the reaction mixture was diluted with EtOAc (50 mL). The organic layer was washed with water (20 mL) and brine (20 mL), dried over anhydrous Na₂SO₄ and concentrated under reduced pressure to obtain 2-bromo-5-(cis-3-(trifluoromethoxy)cyclobutyl)-1,3,4-oxadiazole (50 mg, 67% yield) as a yellow solid. LCMS 287.1 [M+H]⁺.

### Step 5: Synthesis of cis-ethyl 1-(5-(3-(trifluoromethoxy)cyclobutyl)-1,3,4-oxadiazol-2-yl)piperidine-4-carboxylate

To a stirred mixture of 2-bromo-5-(cis-3-(trifluoromethoxy)cyclobutyl)-1,3,4-oxadiazole (50 mg, 0.174 mmol, 1.0 eq.) and ethyl piperidine-4-carboxylate (27 mg, 0.174 mmol, 1.0 eq.) in DMF (1 mL) was added potassium carbonate (48 mg, 0.348 mmol, 2.0 eq.). The resulting reaction mixture was stirred at 90° C. for 2 h. The product formation was confirmed by LCMS. After completion of reaction the reaction mixture was diluted with EtOAc (10 mL). The organic layer was washed with water (5×5 mL) and brine (5 mL), dried over anhydrous Na₂SO₄ and concentrated under reduced pressure. The crude product was purified by flash chromatography to obtain cis-ethyl 1-(5-(3-(trifluoromethoxy)cyclobutyl)-1,3,4-oxadiazol-2-yl)piperidine-4-carboxylate (40 mg, 63% yield) as a yellow solid. LCMS 364.4 [M+H]⁺.

### Step 6: Synthesis of 1-(5-(cis-3-(trifluoromethoxy)cyclobutyl)-1,3,4-oxadiazol-2-yl)piperidine-4-carboxylic acid

To a stirred solution of ethyl 1-(5-(cis-3-(trifluoromethoxy)cyclobutyl)-1,3,4-oxadiazol-2-yl)piperidine-4-carboxylate (40 mg, 0.11 mmol, 1 eq.) in THF (5 mL) was added LiOH.H₂O (13 mg, 0.330 mmol, 3.0 eq.) in water (5 mL) and the resulting reaction mixture was stirred at RT for 1 h. The product formation was confirmed by LCMS. After completion of reaction the reaction mixture was concentrated under reduced pressure. The residue was acidified with 1 M HCl (pH˜3) and extracted with EtOAc (20 mL). The organic layer was dried over anhydrous Na₂SO₄ and concentrated under reduced pressure to obtain 1-(5-(cis-3-(trifluoromethoxy)cyclobutyl)-1,3,4-oxadiazol-2-yl)piperidine-4-carboxylic acid (25 mg, 67% yield) as an off white solid. LCMS 336.3 [M+H]⁺.

### Step 7: Synthesis of N-(6-chloroquinolin-2-yl)-1-(5-(cis-3-(trifluoromethoxy)cyclobutyl)-1,3,4-oxadiazol-2-yl)piperidine-4-carboxamide

To a stirred mixture of 1-(5-(cis-3-(trifluoromethoxy)cyclobutyl)-1,3,4-oxadiazol-2-yl)piperidine-4-carboxylic acid (65 mg, 0.194 mmol, 1 eq.), 6-chloroquinolin-2-amine (34 mg, 0.194 mmol, 1 eq.) and EDC.HCl (149 mg, 0.774 mmol, 4.0 eq.) in DCM (5 mL) was added DMAP (94 mg, 0.774 mmol, 4.0 eq.). The resulting reaction mixture was stirred at RT overnight. The product formation was confirmed by LCMS. After completion of reaction the reaction mixture was diluted with DCM (20 mL). The organic layer was washed with saturated citric acid (10 mL) and brine (10 mL), dried over anhydrous Na₂SO₄ and concentrated under reduced pressure. The crude product was purified by reverse phase HPLC to obtain N-(6-chloroquinolin-2-yl)-1-(5-(cis-3-(trifluoromethoxy)cyclobutyl)-1,3,4-oxadiazol-2-yl)piperidine-4-carboxamide (Compound 50-3 mg, 3% yield) as a white solid. LCMS 496.5 [M+H]⁺; ¹H NMR (400 MHz, DMSO-d₆) δ 10.93 (s, 1H), 8.33 (s, 2H), 8.05 (s, 1H), 7.81 (d, J=8.8 Hz, 1H), 7.74-7.67 (m, 1H), 4.90-4.80 (m, 1H), 3.89 (d, J=11.8 Hz, 2H), 3.05 (t, J=11.6 Hz, 3H), 2.80 (d, J=9.6 Hz, 3H), 1.92 (d, J=14.9 Hz, 3H), 1.75-1.57 (m, 3H).

### Example 48

### Synthesis of trans-N-((5-chlorobenzofuran-2-yl)methyl)-4-(5-(4-chlorophenyl)-1,3,4-oxadiazol-2-yl)cyclohexanecarboxamide

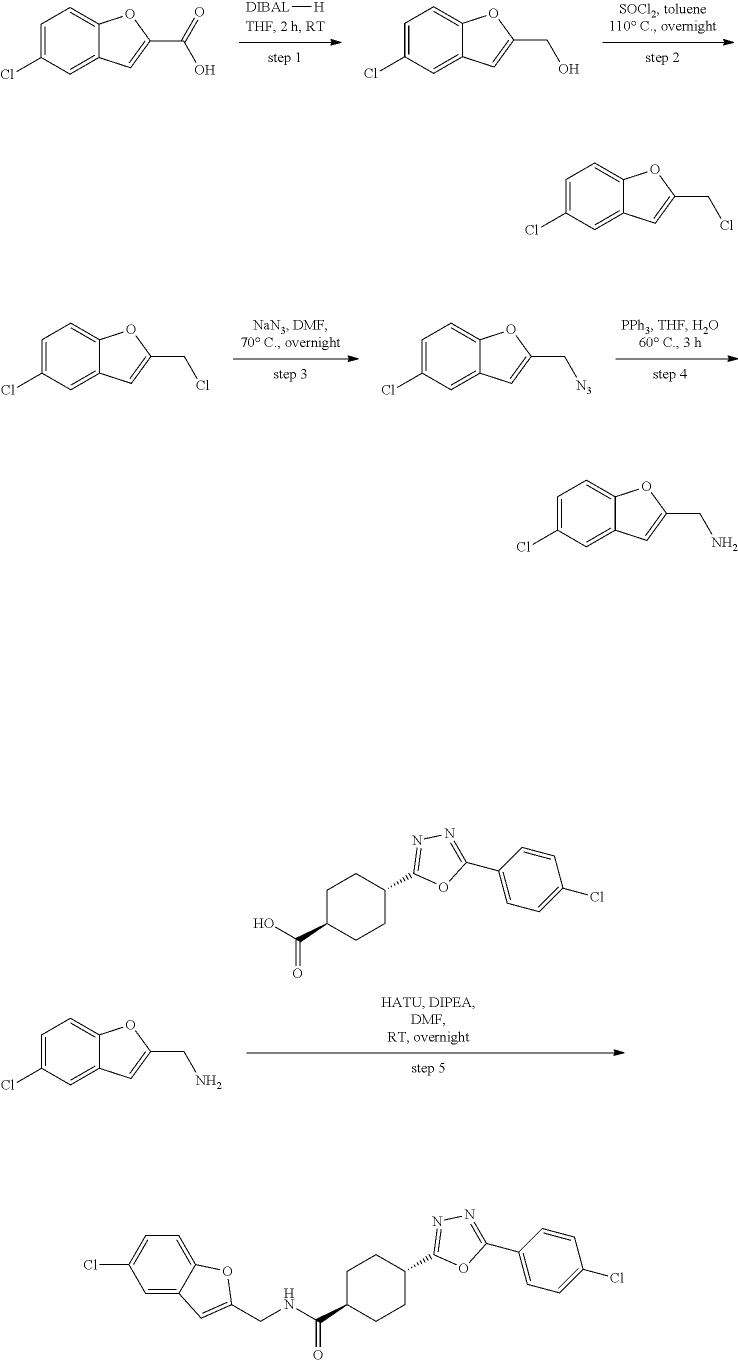

### Step 1: Synthesis of (5-chlorobenzofuran-2-yl)methanol

To a stirred mixture of 5-chlorobenzofuran-2-carboxylic acid (3000 mg, 15.3 mmol, 1.0 eq.) in THE (40 mL) was added DIBAL-H (50 mL, 76.53 mmol, 5.0 eq.) at −78° C. and the resulting reaction mixture was stirred at same temperature for 30 min and then allowed to stir at RT for 2 hours. The product formation was confirmed by NMR. After completion of reaction the reaction mixture was quenched with a saturated solution of NH₄Cl (20 ml). The resulting mixture was acidified with 1 M HCl (pH˜2) and extracted with EtOAc (30 ml×2). The combined organic layers were dried over Na₂SO₄ and concentrated under reduced pressure to obtain (5-chlorobenzofuran-2-yl)methanol (2500 mg, 90% yield) as a brown solid. ¹H NMR (400 MHz, DMSO-d₆) δ 7.69 (s, 1H), 7.60-7.57 (d, 1H), 6.75 (s, 1H), 5.52-5.48 (m, 1H), 4.59-4.53 (d, 2H).

### Step 2: Synthesis of 5-chloro-2-(chloromethyl)benzofuran

To a stirred solution of (5-chlorobenzofuran-2-yl)methanol (3100 mg, 17.02 mmol, 1.0 eq.) in toluene (15 mL) was added SOCl₂ (6080 mg, 51.09 mmol, 3.0 eq.) and the resulting reaction mixture was refluxed overnight at 110° C. The product formation was confirmed by NMR. After completion of reaction the reaction mixture was concentrated under reduced pressure. The crude product was purified by flash chromatography (0-30% ethyl acetate in hexane as eluent) to obtain 5-chloro-2-(chloromethyl)benzofuran (2100 mg, 62% yield) as a brown oil. ¹H NMR (400 MHz, DMSO-d₆) δ 7.75 (s, 1H), 7.67-7.61 (d, 1H), 7.39-7.33 (d, 1H), 7.10 (s, 1H), 4.98 (s, 2H).

### Step 3: Synthesis of 2-(azidomethyl)-5-chlorobenzofuran

To a stirred solution of 5-chloro-2-(chloromethyl)benzofuran (2100 mg, 10.4 mmol, 1 eq.) in DMF (10 mL) was added NaN₃ (1350 mg, 20.8 mmol, 1.9 eq.) and the resulting reaction mixture was heated at 70° C. overnight. The product formation was confirmed by NMR. After completion of reaction the reaction mixture was quenched with water (20 mL) and extracted with EtOAc (30 ml×2). The combined organic layers were dried over Na₂SO₄ and concentrated under reduced pressure. The crude product was purified by flash chromatography (0-30% ethyl acetate in hexane as eluent) to obtain 2-(azidomethyl)-5-chlorobenzofuran (1300 mg, 96% yield) as a brown solid. ¹H NMR (400 MHz, DMSO-d₆) δ 7.70 (s, 1H), 7.63-7.58 (d, 1H), 7.37-7.30 (d, 1H), 6.93 (s, 1H), 4.53 (s, 2H).

### Step 4: Synthesis of (5-chlorobenzofuran-2-yl)methanamine

To a stirred solution of 2-(azidomethyl)-5-chlorobenzofuran (1300 mg, 6.28 mmol, 1.0 eq.) in THE (30 mL) and water (5 mL) was added PPh₃ (1800 mg, 6.9 mmol, 1.1 eq.) and the resulting reaction mixture was heated at 60° C. for 3 hours. The product formation was confirmed by LCMS. After completion of reaction the reaction mixture was concentrated under reduced pressure. The crude product was purified by flash chromatography (0-5% MeOH in DCM as eluent) to obtain (5-chlorobenzofuran-2-yl)methanamine (700 mg, 87% yield) as brown solid. LCMS 182.2 [M+H]⁺.

### Step 5: Synthesis of trans-N-((5-chlorobenzofuran-2-yl)methyl)-4-(5-(4-chlorophenyl)-1,3,4-oxadiazol-2-yl)cyclohexanecarboxamide

To a stirred mixture of trans-4-(5-(4-chlorophenyl)-1,3,4-oxadiazol-2-yl)cyclohexanecarboxylic acid (20 mg, 0.065 mmol, 1 eq.), (5-chlorobenzofuran-2-yl)methanamine (12 mg, 0.065 mmol, 1.2 eq.) and HATU (37 mg, 0.097 mmol, 1.5 eq.) in DMF (2 mL) was added DIPEA (34 mg, 0.2608 mmol, 4.0 eq.). The resulting reaction mixture was stirred at RT overnight. The product formation was confirmed by LCMS. After completion of reaction the reaction mixture was poured into ice cold water (10 mL). The resulting solid was filtered off and dried under vacuum. The crude product was washed with pentane to obtain trans-N-((5-chlorobenzofuran-2-yl)methyl)-4-(5-(4-chlorophenyl)-1,3,4-oxadiazol-2-yl)cyclohexanecarboxamide (Compound 51—12 mg, 39% yield) as a white solid. LCMS 470.5 [M+H]⁺; ¹H NMR (400 MHz, DMSO-d₆) δ 8.47 (s, 1H), 8.00 (d, J=8.3 Hz, 2H), 7.71-7.64 (m, 3H), 7.57 (d, J=9.2 Hz, 1H), 7.28 (dd, J=2.0, 8.6 Hz, 1H), 6.68 (s, 1H), 4.43 (d, J=5.3 Hz, 2H), 3.02 (br. s., 1H), 2.33 (br. s., 1H), 2.19 (br. s., 2H), 1.90 (br. s., 2H), 1.67-1.47 (m, 4H).

### Example 49

### Synthesis of trans-N-(5-chlorobenzo[d]thiazol-2-yl)-4-(5-(cis-3-(trifluoromethoxy)cyclobutyl)-1,3,4-oxadiazol-2-yl)cyclohexanecarboxamide

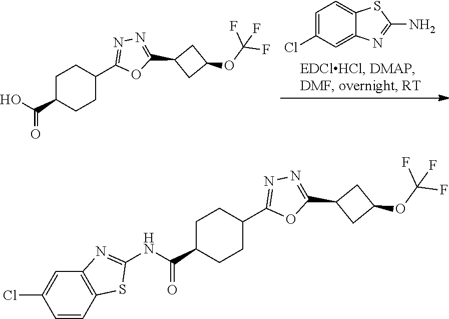

To a stirred solution of trans-4-(5-(cis-3-(trifluoromethoxy)cyclobutyl)-1,3,4-oxadiazol-2-yl)cyclohexanecarboxylic acid (40 mg, 0.1197 mmol, 1.0 eq.) in DCM (5 mL) was added EDCI.HCl (92 mg, 0.479 mmol, 4.0 eq.) and DMAP (58 mg, 0.479 mmol, 4.0 eq.). The resulting reaction mixture was stirred at RT for 15 minutes followed by the addition of 5-chlorobenzo[d]thiazol-2-amine (22 mg, 0.1197 mmol, 1.0 eq.) at RT and allowed to stir at RT overnight. The product formation was confirmed by LCMS and TLC. After completion of reaction the reaction mixture was diluted with DCM (20 mL). The organic layer was washed with 1 M HCl solution (10 mL), brine (10 mL), dried over anhydrous Na₂SO₄ and concentrated under reduced pressure. The crude product was crystallized in methanol to obtain trans-N-(5-chlorobenzo[d]thiazol-2-yl)-4-(5-(cis-3-(trifluoromethoxy)cyclobutyl)-1,3,4-oxadiazol-2-yl)cyclohexanecarboxamide (Compound 52-30 mg, 50% yield) as a white solid. LCMS 531.5 [M+H]⁺; ¹H NMR (400 MHz, DMSO-d₆) δ 12.50 (s, 1H), 8.01 (d, J=8.3 Hz, 1H), 7.82-7.70 (m, 1H), 7.34 (dd, J=1.5, 8.6 Hz, 1H), 5.02-4.83 (m, 1H), 3.41 (d, J=7.9 Hz, 2H), 2.96 (br. s., 1H), 2.84 (d, J=9.6 Hz, 2H), 2.61 (br. s., 2H), 2.15 (d, J=11.8 Hz, 2H), 2.02 (d, J=13.2 Hz, 2H), 1.72-1.42 (m, 4H).

### Example 50

### Synthesis of trans-N-((5-chlorobenzofuran-2-yl)methyl)-4-(5-((4-chlorophenoxy)methyl)-1,3,4-oxadiazol-2-yl)cyclohexanecarboxamide

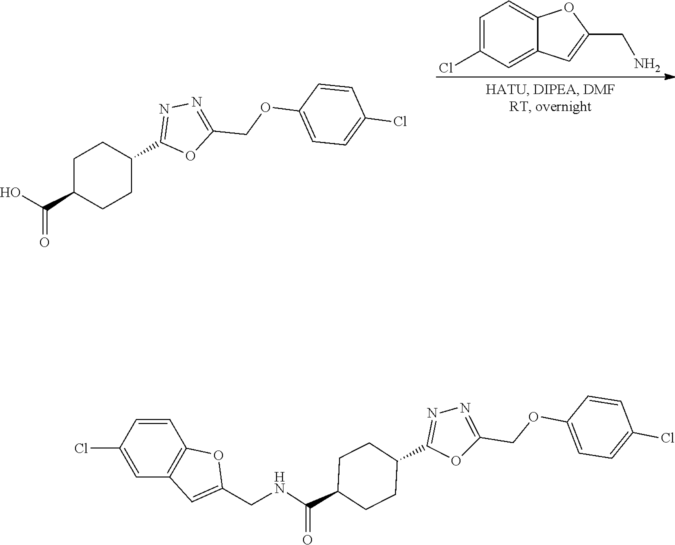 [SMILES: NCc1cc2cc(Cl)ccc2o1 ; O=C(O)[C@H]1CC[C@H](c2nnc(COc3ccc(Cl)cc3)o2)CC1 ; O=C(NCc1cc2cc(Cl)ccc2o1)[C@H]1CC[C@H](c2nnc(COc3ccc(Cl)cc3)o2)CC1]

To a stirred mixture of trans-4-(5-((4-chlorophenoxy)methyl)-1,3,4-oxadiazol-2-yl)cyclohexanecarboxylic acid (20 mg, 0.0593 mmol, 1 eq.), (5-chlorobenzofuran-2-yl)methanamine (11 mg, 0.0593 mmol, 1.0 eq.) and HATU (34 mg, 0.0895 mmol, 1.5 eq.) in DMF (2 mL) was added DIPEA (0.42 mL, 0.238 mmol, 4.0 eq.). The resulting reaction mixture was stirred at RT overnight. The product formation was confirmed by LCMS. After completion of reaction the reaction mixture was poured into ice cold water (10 ml). The resulting solid was filtered off and dried under vacuum. The crude product was washed with pentane to obtain trans-N-((5-chlorobenzofuran-2-yl)methyl)-4-(5-((4-chlorophenoxy)methyl)-1,3,4-oxadiazol-2-yl)cyclohexanecarboxamide (Compound 53-8 mg, 27% yield) as an off white solid. LCMS 500.5 [M+H]⁺; ¹H NMR (400 MHz, DMSO-d₆) δ 8.44 (br. s., 1H), 7.64 (s, 1H), 7.55 (d, J=8.8 Hz, 1H), 7.36 (d, J=9.2 Hz, 2H), 7.26 (d, J=8.8 Hz, 1H), 7.07 (d, J=8.8 Hz, 2H), 6.65 (s, 1H), 5.36 (s, 2H), 4.40 (d, J=4.8 Hz, 2H), 2.96 (br. s., 1H), 2.24 (br. s., 1H), 2.09 (d, J=8.8 Hz, 2H), 1.84 (br. s., 2H), 1.55-1.35 (m, 4H).

### Example 51

### Synthesis of trans-4-(5-(5-chlorobenzofuran-2-yl)-1,3,4-oxadiazol-2-yl)-N-((5-chlorobenzofuran-2-yl)methyl)cyclohexanecarboxamide

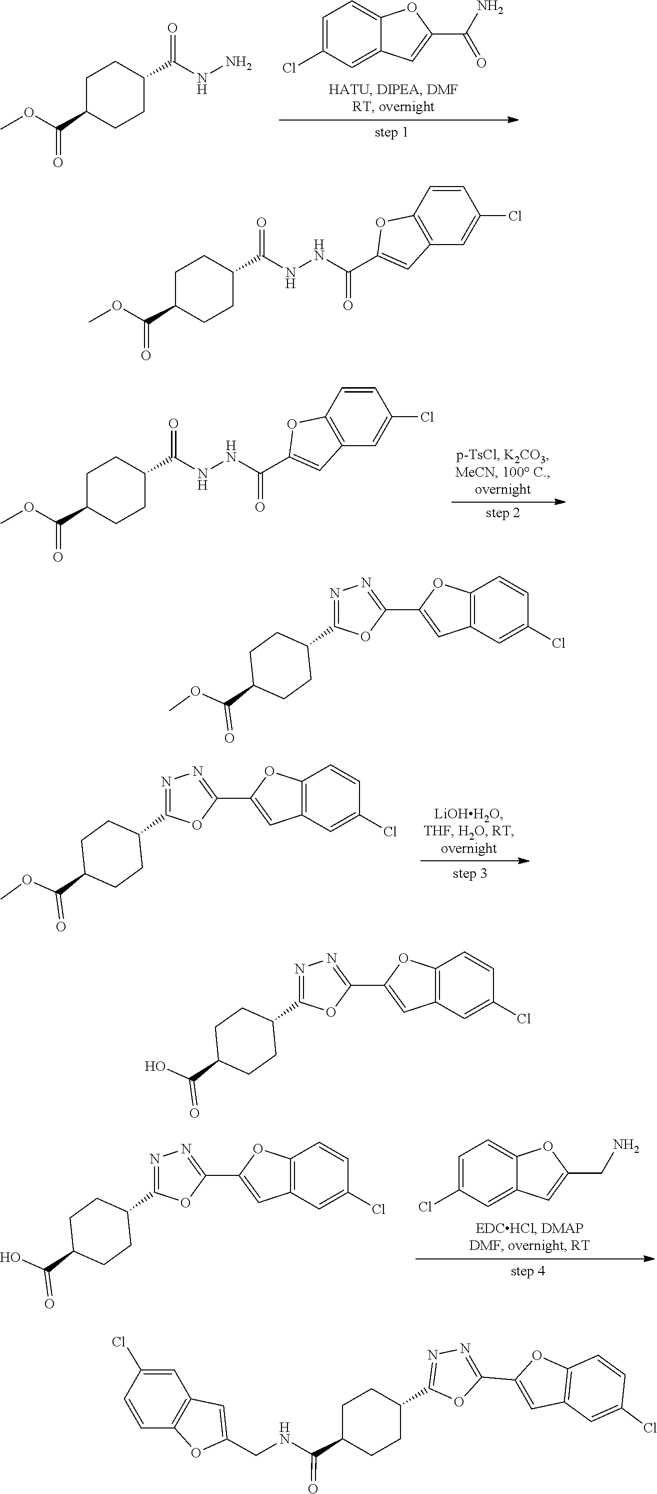

### Step 1: Synthesis of trans-methyl 4-(2-(5-chlorobenzofuran-2-carbonyl)hydrazinecarbonyl)cyclohexanecarboxylate

To a solution of trans-methyl 4-(hydrazinecarbonyl)cyclohexanecarboxylate (100 mg, 0.5 mmol, 1.0 eq.), 5-chlorobenzofuran-2-carboxylic acid (117 mg, 0.6 mmol, 1.2 eq.) and HATU (380 mg, 2.0 mmol, 2.0 eq.) in DMF (1.0 mL) was added DIPEA (129 mg, 1 mmol, 2.0 eq.) was added. The resulting reaction mixture was allowed to stir at RT overnight. Progress of the reaction was monitored by LCMS. The reaction mixture was diluted with cold water (50 mL) and the resulting solid was filtered off, washed with water and dried under vacuum to obtain trans-methyl 4-(2-(5-chlorobenzofuran-2-carbonyl)hydrazinecarbonyl)cyclohexanecarboxylate (100 mg, 52% yield) as an off-white solid. LCMS 379.3 [M+H]⁺; ¹H NMR (400 MHz, DMSO-d₆) δ 10.98 (br. s., 1 H) 10.62 (br. s., 1H) 7.94 (d, J=1.75 Hz, 1H) 7.73-7.82 (m, 1H) 7.62 (s, 1H) 7.45-7.55 (m, 1H) 3.43-3.70 (m, 3H) 2.39-2.17 (m, 2H) 1.89-1.84 (m, 2H) 1.75 (d, J=13.59 Hz, 1H) 1.29-1.53 (m, 1H) 1.23 (d, J=6.58 Hz, 4H).

### Step 2: Synthesis of trans-methyl 4-(5-(5-chlorobenzofuran-2-yl)-1,3,4-oxadiazol-2-yl)cyclohexanecarboxylate

To a stirred mixture of trans-methyl 4-(2-(5-chlorobenzofuran-2-carbonyl)hydrazinecarbonyl)cyclohexanecarboxylate (200 mg, 0.529 mmol, 1.0 eq.), K₂CO₃ (219 mg, 1.587 mmol, 3.0 eq.) and molecular sieves (200 mg) in acetonitrile (10 mL) was added 4-toluenesulfonyl chloride (251 mg, 1.322 mmol, 2.5 eq.) and the resulting reaction mixture was heated at 100° C. overnight. The product formation was confirmed by LCMS and TLC. After completion of reaction the reaction mixture was filtered and the filtrate was diluted with EtOAc (25 mL). The organic layer was washed with water (20 mL) and brine (20 mL), dried over anhydrous Na₂SO₄ and concentrated under reduced pressure to obtain trans-methyl 4-(5-(5-chlorobenzofuran-2-yl)-1,3,4-oxadiazol-2-yl)cyclohexanecarboxylate (200 mg, quant. yield) as an off white solid. LCMS 361.2 [M+H]⁺; ¹H NMR (400 MHz, DMSO-d₆) δ 7.83 (d, J=9.21 Hz, 1H) 7.70-7.80 (m, 1H) 7.48 (m, J=7.89 Hz, 1H) 7.02-7.19 (m, 1H) 3.53-3.70 (m, 3H) 3.50 (br. s., 1H) 2.25-2.34 (m, 1H) 2.11-2.22 (m, 2H) 2.03 (d, J=12.72 Hz, 2H) 1.41-1.70 (m, 4H).

### Step 3: Synthesis of trans-4-(5-(5-chlorobenzofuran-2-yl)-1,3,4-oxadiazol-2-yl)cyclohexanecarboxylic acid

To a stirred solution of trans-methyl 4-(5-(5-chlorobenzofuran-2-yl)-1,3,4-oxadiazol-2-yl)cyclohexanecarboxylate (150 mg, 0.416 mmol, 1.0 eq.) in THE (2 mL) was added LiOH.H2O (26 mg, 0.624 mmol, 1.5 eq.) and H₂O (2 ml) and the resulting reaction mixture was stirred at RT overnight. The product formation was confirmed by LCMS. After completion of reaction, the reaction mixture was concentrated under reduced pressure and the residue was dissolved in water (10 mL). The aqueous layer was acidified with 1 M HCl. The resulting solid was filtered off and dried under vacuum to obtain trans-4-(5-(5-chlorobenzofuran-2-yl)-1,3,4-oxadiazol-2-yl)cyclohexanecarboxylic acid (120 mg, 83% yield) as an off white solid. LCMS 347.3 [M+H]⁺; ¹H NMR (400 MHz, DMSO-d₆) δ 12.07 (br. s., 1H) 7.84-7.95 (m, 1H) 7.82 (d, J=8.77 Hz, 1H) 7.74 (s, 1H) 7.46-7.58 (m, 1H) 3.04 (br. s., 1H) 2.31 (br. s., 1H) 2.16 (d, J=10.96 Hz, 2H) 2.01 (d, J=13.15 Hz, 2H) 1.38-1.68 (m, 4H).

### Step 4: Synthesis of trans-4-(5-(5-chlorobenzofuran-2-yl)-1,3,4-oxadiazol-2-yl)-N-((5-chlorobenzofuran-2-yl)methyl)cyclohexanecarboxamide

To a stirred solution of trans-4-(5-(5-chlorobenzofuran-2-yl)-1,3,4-oxadiazol-2-yl)cyclohexanecarboxylic acid (30 mg, 0.086 mmol, 1.0 eq.), DMAP (41 mg, 0.344 mmol, 4.0 equiv) and EDCl.HCl (66 mg, 0.344 mmol, 4.0 eq.) in DCM (5 mL) was added (5-chlorobenzofuran-2-yl)methanamine (31 mg, 0.172 mmol, 2.0 eq.). The resulting reaction mixture was stirred at RT overnight. Product formation was confirmed by LCMS. After completion of reaction the reaction mixture diluted with water (10 mL) and extracted with DCM (25 ml×2). The combined organic layers were washed with brine, dried over Na₂SO₄ and concentrated under reduced pressure. The crude product was purified by flash chromatography (0-2.5% MeOH in DCM as an eluent) to obtain trans-4-(5-(5-chlorobenzofuran-2-yl)-1,3,4-oxadiazol-2-yl)-N-((5-chlorobenzofuran-2-yl)methyl)cyclohexanecarboxamide (Compound 54-5 mg, 11% yield) as an off white solid. LCMS 510.5 [M+H]⁺; ¹H NMR (400 MHz, DMSO-d₆) δ 8.48 (d, J=5.26 Hz, 1H) 7.90 (s, 1H) 7.84 (d, J=9.21 Hz, 1H) 7.76 (s, 1H) 7.52-7.64 (m, 3H) 7.28 (d, J=8.77 Hz, 1H) 6.68 (s, 1H) 4.44 (d, J=5.70 Hz, 2H) 3.07 (br. s., 1H) 2.33 (br. s., 1H) 2.20 (br. s., 2H) 1.93 (br. s., 2H) 1.59 (t, J=10.52 Hz, 4H).

### Example 52

### Synthesis of trans-4-(5-(4-chlorobenzyl)-1,3,4-oxadiazol-2-yl)-N-(6-chloroquinolin-2-yl)cyclohexanecarboxamide

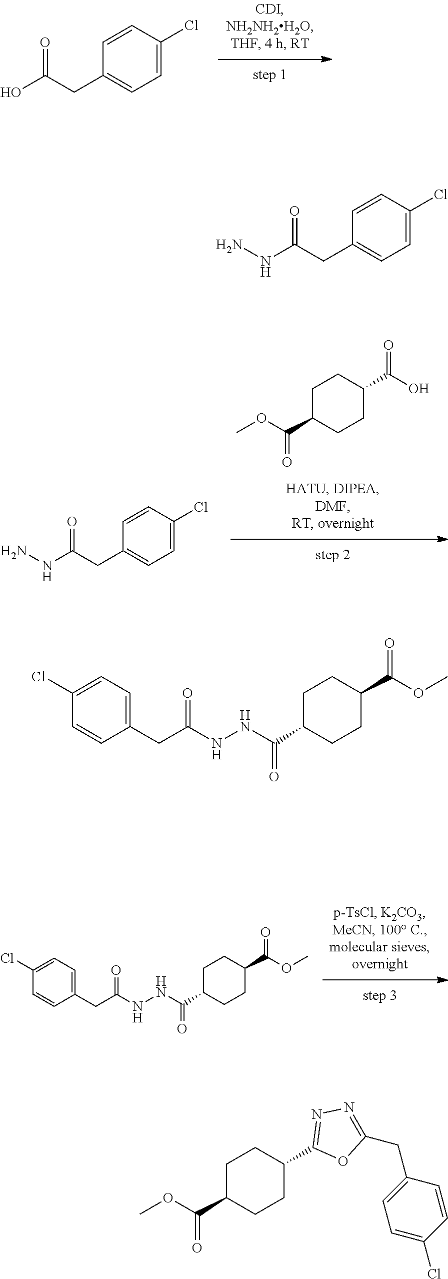

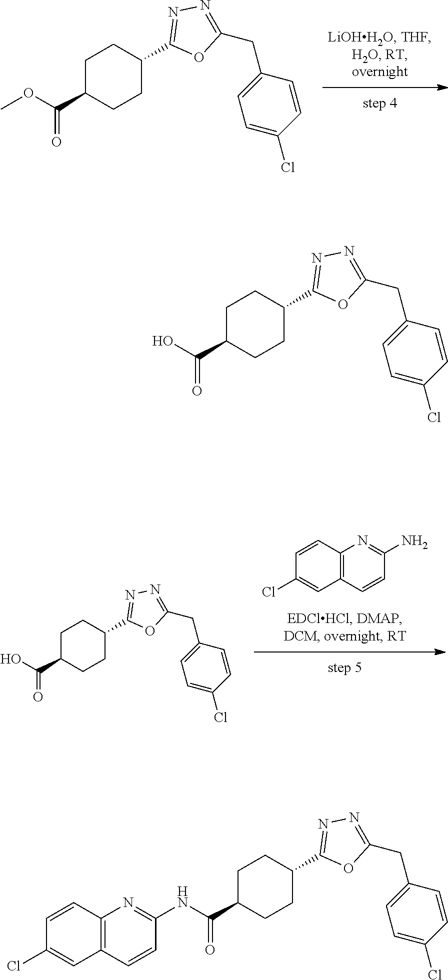 [SMILES: COC(=O)[C@H]1CC[C@H](c2nnc(Cc3ccc(Cl)cc3)o2)CC1 ; O=C(O)[C@H]1CC[C@H](c2nnc(Cc3ccc(Cl)cc3)o2)CC1 ; O=C(O)[C@H]1CC[C@H](c2nnc(Cc3ccc(Cl)cc3)o2)CC1 ; Nc1ccc2cc(Cl)ccc2n1 ; O=C(Nc1ccc2cc(Cl)ccc2n1)[C@H]1CC[C@H](c2nnc(Cc3ccc(Cl)cc3)o2)CC1]

### Step 1: Synthesis of 2-(4-chlorophenyl)acetohydrazide

To a stirred solution of 2-(4-chlorophenyl)acetohydrazide (500 mg, 2.93 mmol, 1.0 equiv) in THF (50 mL) was added 1,1′-Carbonyldiimidazole (712 mg, 4.396 mmol, 1.5 equiv) and the resulting reaction mixture was stirred at RT for 2 h. Hydrazine hydrate (0.3 mL, 5.86 mmol, 2.0 equiv) was added drop wise and allowed to stir at RT for 2 h. The product formation was confirmed by LCMS. After completion of reaction, the reaction mixture was concentrated under reduced pressure. The crude product was washed with in hexane, dried under vacuum to obtain 2-(4-chlorophenyl)acetohydrazide (435 mg, 80.7% yield) as a white solid. LCMS 185.1 [M+H]⁺.

### Step 2: Synthesis of trans-methyl 4-(2-(2-(4-chlorophenyl)acetyl)hydrazinecarbonyl)cyclohexanecarboxylate

To a stirred mixture of trans-4-(methoxycarbonyl)cyclohexanecarboxylic acid (200 mg, 1.075 mmol, 1.0 equiv), 2-(4-chlorophenyl)acetohydrazide (237 mg, 1.29 mmol, 1.2 equiv) & HATU (612 mg, 1.613 mmol, 1.5 equiv) in DMF (5 mL) was added DIPEA (0.8 mL, 4.3 mmol, 4.0 equiv) and the resulting reaction mixture was stirred at RT overnight. The product formation was confirmed by LCMS. After completion of reaction, the reaction mixture was poured into ice cold water (100 ml). The resulting off white solid was filtered off, washed with ether and dried under vacuum to obtain trans-methyl 4-(2-(2-(4-chlorophenyl)acetyl)hydrazinecarbonyl)cyclohexanecarboxylate (200 mg, 53% yield) as an off white solid. LCMS 353.2 [M+H]⁺.

### Step 3: Synthesis of trans-methyl 4-(5-(4-chlorobenzyl)-1,3,4-oxadiazol-2-yl)cyclohexanecarboxylate

To a stirred mixture of trans-methyl 4-(2-(2-(4-chlorophenyl)acetyl)hydrazinecarbonyl)cyclohexanecarboxylate (200 mg, 0.568 mmol, 1.0 equiv), K₂CO₃ (392 mg, 2.84 mmol, 5.0 equiv) & molecular sieves (200 mg) in acetonitrile (20 mL) was added 4-toluenesulfonyl chloride (270 mg, 1.42 mmol, 2.5 equiv). The resulting reaction mixture was stirred at 100° C. overnight. The product formation was confirmed by LCMS. After completion of reaction, the reaction mixture filtered off. The filtrate was diluted with EtOAc (50 mL). Organic layer was washed with water (50 mL) & brine (50 mL), dried over anhydrous Na₂SO₄ and concentrated under reduced pressure to obtain trans-methyl 4-(5-(4-chlorobenzyl)-1,3,4-oxadiazol-2-yl)cyclohexanecarboxylate (150 mg, 79% yield) as an off white solid. LCMS 335.2 [M+H]⁺.

### Step 4: Synthesis of trans-4-(5-(4-chlorobenzyl)-1,3,4-oxadiazol-2-yl)cyclohexanecarboxylic acid

To a stirred solution of trans-methyl 4-(5-(4-chlorobenzyl)-1,3,4-oxadiazol-2-yl)cyclohexanecarboxylate (150 mg, 0.449 mmol, 1.0 equiv) in THF (4 mL) & H₂O (4 mL) was added Lithium hydroxide monohydrate (57 mg, 1.347 mmol, 1.0 equiv). The resulting reaction mixture was stirred at RT for overnight. The product formation was confirmed by LCMS. After completion of reaction, the reaction mixture was concentrated under reduced pressure and crude product dissolved in water (10 mL), and acidified with 1 M HCl. The resulting solid was filtered off and dried under vacuum to obtain trans-4-(5-(4-chlorobenzyl)-1,3,4-oxadiazol-2-yl)cyclohexanecarboxylic acid (80 mg, 56% yield) as a white solid. LCMS 321.2 [M+H]⁺.

### Step 5: Synthesis of trans-4-(5-(4-chlorobenzyl)-1,3,4-oxadiazol-2-yl)-N-(6-chloroquinolin-2-yl)cyclohexanecarboxamide

To a stirred solution of trans-4-(5-(4-chlorobenzyl)-1,3,4-oxadiazol-2-yl)cyclohexanecarboxylic acid (50 mg, 0.156 mmol, 1 equiv), EDCI.HCl (45 mg, 0.234 mmol, 1 equiv) & DMAP (29 mg, 0.234 mmol, 1.5 equiv) in DCM (10 mL) was added 6-chloroquinolin-2-amine (22 mg, 0.125 mmol, 0.8 equiv). The resulting reaction mixture was stirred at RT for overnight. The product formation was confirmed by LCMS. After completion of reaction, the mixture was concentrated under reduced pressure and diluted with EtOAc (20 mL). Organic layer was washed with water (5 mL) & brine (10 mL), dried over anhydrous Na₂SO₄ and concentrated. The crude product was crystallized in methanol to obtain trans-4-(5-(4-chlorobenzyl)-1,3,4-oxadiazol-2-yl)-N-(6-chloroquinolin-2-yl)cyclohexanecarboxamide (Compound 237-14 mg, 18.7% yield) as a white solid. LCMS 481.4 [M+H]⁺. ¹H NMR (400 MHz, DMSO-d₆) δ 10.84 (s, 1H), 8.45-8.23 (m, 2H), 8.05 (d, J=2.2 Hz, 1H), 7.80 (d, J=8.8 Hz, 1H), 7.71 (dd, J=2.2, 9.2 Hz, 1H), 7.43 (d, J=8.3 Hz, 2H), 7.35 (d, J=8.8 Hz, 2H), 4.26 (s, 2H), 2.94 (br. s., 1H), 2.61 (br. s., 1H), 2.10 (d, J=13.6 Hz, 2H), 1.97 (d, J=12.7 Hz, 2H), 1.65-1.40 (m, 4H).

### Example 53

### Synthesis of 2-(4-chloro-3-fluorophenoxy)-N-(trans-4-(5-(4-chlorobenzyl)-1,3,4-oxadiazol-2-yl)cyclohexyl)acetamide

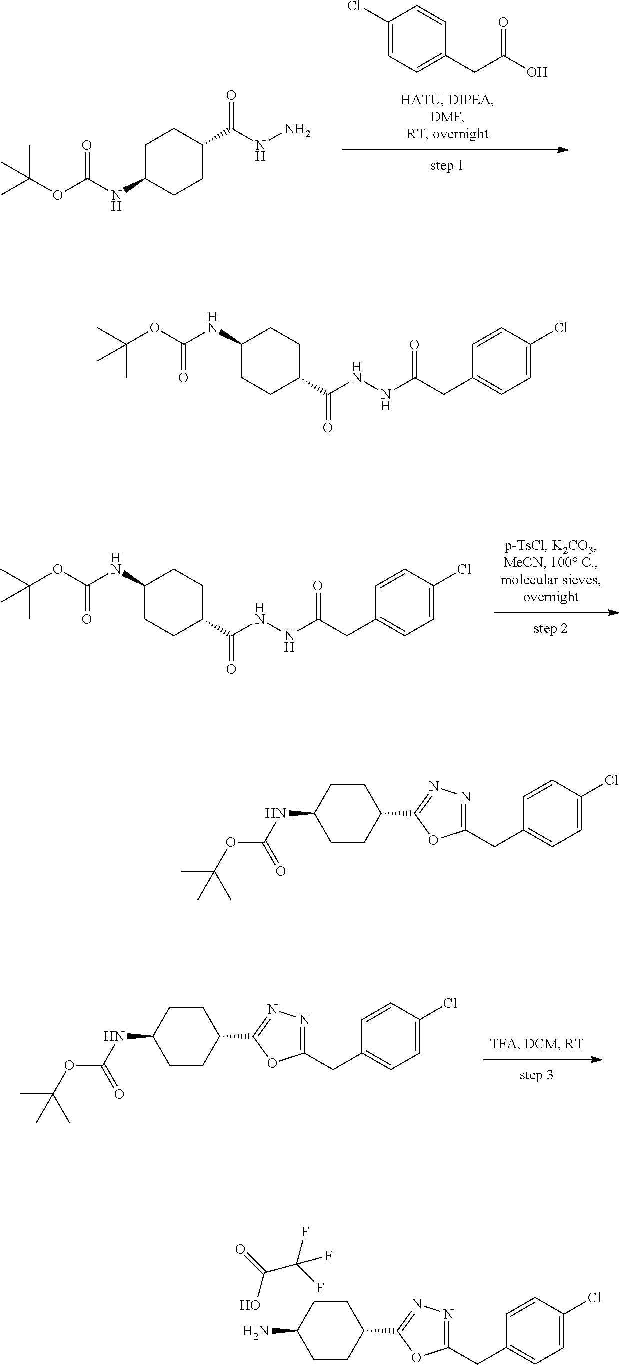

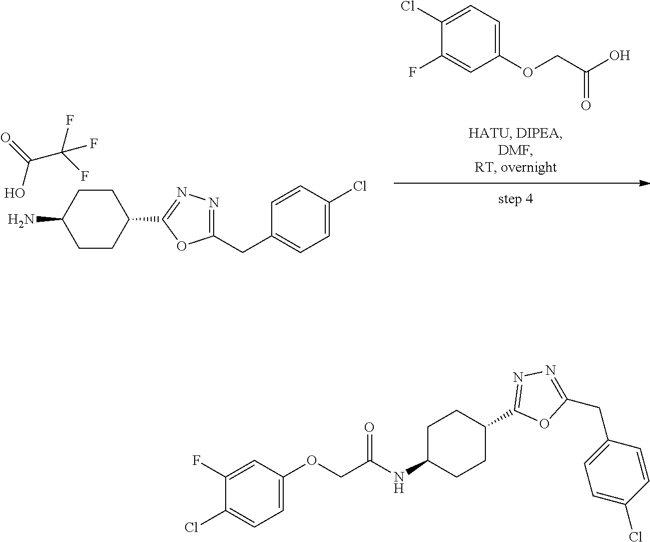

### Step 1: Synthesis of tert-butyl trans-4-(2-(2-(4-chlorophenyl)acetyl)hydrazinecarbonyl)cyclohexylcarbamate

To a stirred solution of trans-tert-butyl 4-(hydrazinecarbonyl)cyclohexylcarbamate (500 mg, 1.945 mmol, 1.0 equiv), 2-(4-chlorophenyl)acetic acid (332 mg, 1.945 mmol, 1.0 equiv) and HATU (1108 mg, 2.92 mmol, 1 equiv) in DMF (7 mL) was added DIPEA (1.4 mL, 3.89 mmol, 4.0 equiv). The resulting reaction mixture was stirred at RT for overnight. Product formation was confirmed by LCMS. After completion of reaction, the reaction mixture was poured into ice cold water (100 ml). The resulting solid was filtered off and dried under vacuum to obtain tert-butyl trans-4-(2-(2-(4-chlorophenyl)acetyl)hydrazinecarbonyl)cyclohexylcarbamate (650 mg, 81.76% Yield) as an off white solid. LCMS 410.3 [M+H]⁺. ¹H NMR (400 MHz, DMSO-d₆) δ 10.00 (br. s., 1H), 9.71 (br. s., 1H), 7.36 (d, J=8.3 Hz, 2H), 7.30 (d, J=7.9 Hz, 2H), 6.71 (d, J=7.5 Hz, 1H), 3.45 (s, 2H), 3.16 (br. s., 1H), 2.05 (d, J=11.0 Hz, 1H), 1.78 (br. s., 2H), 1.71 (d, J=11.4 Hz, 2H), 1.37 (m, 11H), 1.22-1.04 (m, 2H).

### Step 2: Synthesis of tert-butyl trans-4-(5-(4-chlorobenzyl)-1,3,4-oxadiazol-2-yl)cyclohexylcarbamate

To a stirred solution of tert-butyl trans-4-(2-(2-(4-chlorophenyl)acetyl)hydrazinecarbonyl)cyclohexylcarbamate (650 mg, 1.589 mmol, 1.0 equiv), K₂CO₃ (1096 mg, 7.95 mmol, 5.0 equiv) and molecular sieves (1000 mg) in acetonitrile (50 mL) was added 4-toluenesulfonyl chloride (755 mg, 3.97 mmol, 2.5 equiv) and the resulting reaction mixture was heated at 100° C. for overnight. Product formation was confirmed by LCMS and TLC. After completion of reaction, the reaction mixture was filtered through Celite®. The filtrate was dissolved in EtOAc (100 mL). The organic layer was washed with water (50 mL) and brine (50 mL), dried over anhydrous Na₂SO₄ and concentrated under reduced pressure to obtain tert-butyl trans-4-(5-(4-chlorobenzyl)-1,3,4-oxadiazol-2-yl)cyclohexylcarbamate (500 mg, 80.51% yield) as an off white solid. LCMS 392.4 [M+H]⁺. ¹H NMR (400 MHz, DMSO-d₆) δ 7.43-7.38 (m, 2H), 7.38-7.29 (m, 2H), 6.81 (d, J=7.9 Hz, 1H), 4.24 (s, 2H), 3.22 (br. s., 1H), 2.78 (t, J=12.3 Hz, 1H), 2.02 (d, J=12.3 Hz, 2H), 1.85 (d, J=11.4 Hz, 2H), 1.54-1.42 (m, 2H), 1.37 (s, 9H), 1.31-1.16 (m, 2H).

### Step 3: Synthesis of trans-4-(5-(4-chlorobenzyl)-1,3,4-oxadiazol-2-yl)cyclohexanamine 2,2,2-trifluoroacetate

To a stirred solution of tert-butyl trans-4-(5-(4-chlorobenzyl)-1,3,4-oxadiazol-2-yl)cyclohexylcarbamate (500 mg, 1.278 mmol, 1.0 equiv) in DCM (40 mL) was added trifluoroacetic acid (2.0 mL) and the resulting reaction mixture was stirred at RT overnight under nitrogen atmosphere. Product formation was confirmed by LCMS. After completion of reaction, the reaction mixture was concentrated under reduced pressure. The crude product was crystallized in diethyl ether and dried under vacuum to obtain trans-4-(5-(4-chlorobenzyl)-1,3,4-oxadiazol-2-yl)cyclohexanamine 2,2,2-trifluoroacetate (500 mg, 96.8% Yield) as a brown solid. LCMS 292.3 [M+H]⁺.

### Step 4: Synthesis of 2-(4-chloro-3-fluorophenoxy)-N-(trans-4-(5-(4-chlorobenzyl)-1,3,4-oxadiazol-2-yl)cyclohexyl)acetamide

To a stirred solution of trans-4-(5-(4-chlorobenzyl)-1,3,4-oxadiazol-2-yl)cyclohexanamine 2,2,2-trifluoroacetate (100 mg, 0.247 mmol, 1 equiv), 20-(4-chloro-3-fluorophenoxy)acetic acid (50 mg, 0.247 mmol, 1.0 equiv) and HATU (141 mg, 0.37 mmol, 1.5 equiv) in DMF (2 mL) was added DIPEA (0.2 mL, 0.988 mmol, 4.0 equiv) and the resulting reaction mixture was stirred at RT overnight. Product formation was confirmed by LCMS and TLC. After completion of reaction, the reaction mixture was poured into ice cold water (100 ml). The resulting solid was filtered off and dried under vacuum to obtain crude product which was crystallized in methanol to obtain 2-(4-chloro-3-fluorophenoxy)-N-(trans-4-(5-(4-chlorobenzyl)-1,3,4-oxadiazol-2-yl)cyclohexyl)acetamide (Compound 238-25 mg, 21.3% Yield) as an off white solid. LCMS 478.5 [M+H]⁺. ¹H NMR (400 MHz, DMSO-d₆) δ 8.02 (d, J=7.9 Hz, 1H), 7.49 (t, J=8.6 Hz, 1H), 7.42 (d, J=7.9 Hz, 2H), 7.34 (d, J=8.3 Hz, 2H), 7.08-7.04 (m, 1H), 6.85 (d, J=11.0 Hz, 1H), 4.51 (s, 2H), 4.25 (s, 2H), 3.64 (br. s., 1H), 2.85 (t, J=11.6 Hz, 1H), 2.05 (d, J=11.8 Hz, 2H), 1.85 (d, J=9.2 Hz, 2H), 1.63-1.48 (m, 2H), 1.44-1.26 (m, 2H).

### Example 54

### Synthesis of 6-chloro-N-(trans-4-(5-(4-chlorobenzyl)-1,3,4-oxadiazol-2-yl)cyclohexyl)quinoline-2-carboxamide

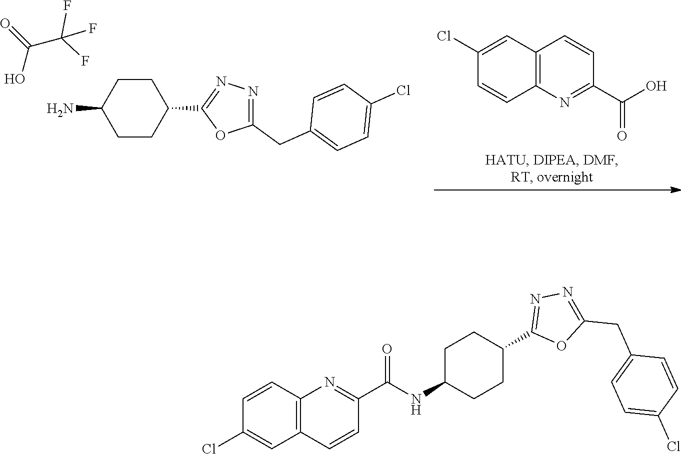

To a stirred solution of trans-4-(5-(4-chlorobenzyl)-1,3,4-oxadiazol-2-yl)cyclohexanamine 2,2,2-trifluoroacetate (100 mg, 0.247 mmol, 1.0 equiv), 6-chloroquinoline-2-carboxylic acid (51 mg, 0.247 mmol, 1 equiv) and HATU (141 mg, 0.37 mmol, 1.5 equiv) in DMF (2 mL) was added DIPEA (0.2 mL, 0.988 mmol, 4.0 equiv) and the resulting reaction mixture was stirred at RT for overnight. Product formation was confirmed by LCMS and TLC. After completion of reaction, the reaction mixture was poured into ice cold water (100 ml). The resulting solid was filtered off and dried under vacuum to obtain crude product which was crystallized in methanol to obtain 6-chloro-N-(trans-4-(5-(4-chlorobenzyl)-1,3,4-oxadiazol-2-yl)cyclohexyl)quinoline-2-carboxamide (Compound 239-25 mg, 21.3% yield) as an off white solid. LCMS 481.5 [M+H]⁺. H NMR (400 MHz, DMSO-d₆) δ 8.69 (d, J=9.2 Hz, 1H), 8.54 (d, J=8.8 Hz, 1H), 8.25 (d, J=2.2 Hz, 1H), 8.18 (dd, J=6.4, 8.6 Hz, 2H), 7.94-7.81 (m, 1H), 7.43 (d, J=8.8 Hz, 2H), 7.36 (d, J=8.3 Hz, 2H), 4.27 (s, 2H), 3.91 (br. s., 1H), 2.90 (br. s., 1H), 2.13 (br. s., 2H), 1.97 (br. s., 2H), 1.68-1.42 (m, 4H).

### Example 55

### Synthesis of 2-(4-chloro-3-fluorophenoxy)-N-(4-(5-((4-chlorophenoxy)methyl)-1,3,4-oxadiazol-2-yl)piperidin-1-yl)acetamide

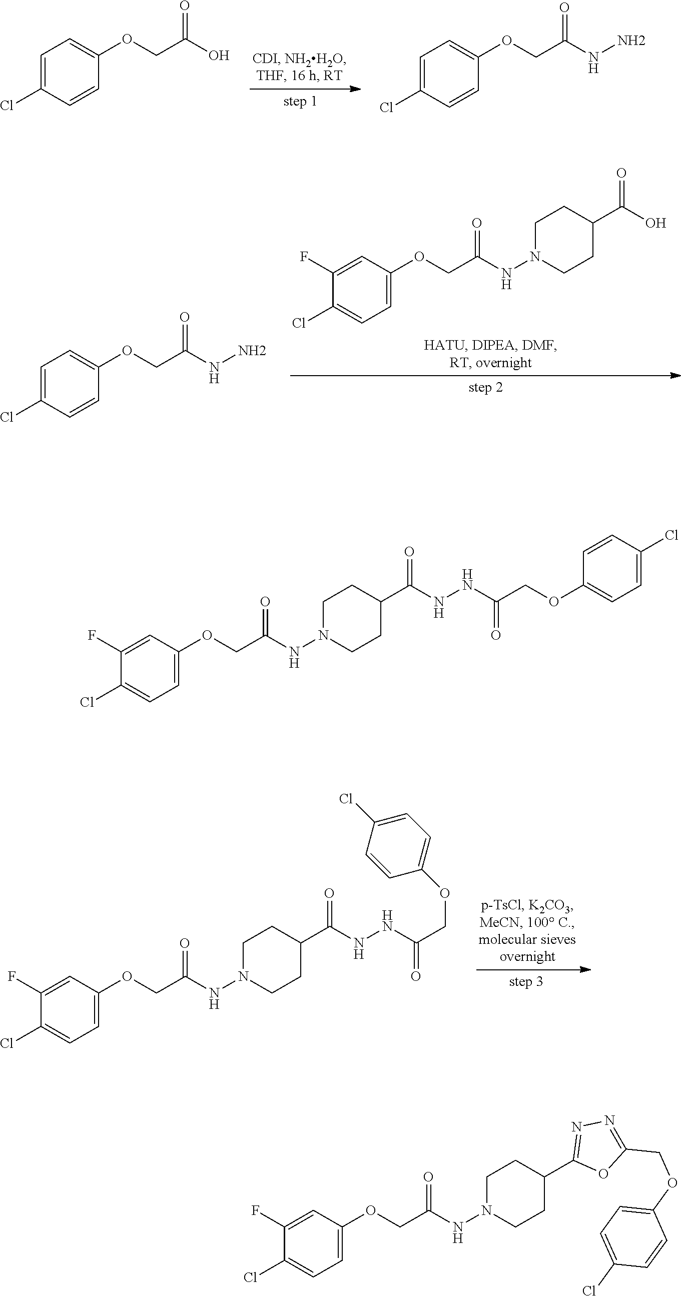

### Step 1: Synthesis of 2-(4-chlorophenoxy)acetohydrazide

To a stirred solution of 2-(4-chlorophenoxy) acetic acid (0.300 g, 1.6078 mmol, 1.0 equiv) in THF (10 mL) was added 1,1′-Carbonyldiimidazole (0.391 g, 2.4117 mmol, 1.5 equiv) and the resulting reaction mixture was stirred at RT for 16 h. Hydrazine hydrate (0.160 g, 3.2156 mmol, 2.0 equiv) was added dropwise. The product formation was confirmed by LCMS. After completion of reaction, the reaction mixture was concentrated under reduced pressure. The crude product was washed with pentane, dried under vacuum to obtain 2-(4-chlorophenoxy)acetohydrazide (0.300 g, 93% yield) as a white solid. LCMS 201.2 [M+H]⁺.

### Step 2: Synthesis of 2-(4-chloro-3-fluorophenoxy)-N-(4-(2-(2-(4-chlorophenoxy)acetyl)hydrazinecarbonyl)piperidin-1-yl)acetamide

To a stirred mixture of 1-(2-(4-chloro-3-fluorophenoxy)acetamido)piperidine-4-carboxylic acid (0.100 g, 0.3023 mmol, 1.0 equiv), 2-(4-chlorophenoxy)acetohydrazide (0.121 g, 0.6047 mmol, 2.0 equiv) & HATU (0.172 mg, 0.4534 mmol, 1.5 equiv) in DMF (3 mL) was added DIPEA (0.156 g, 1.2092 mmol, 4.0 equiv). The resulting reaction mixture was stirred at RT overnight. The product formation was confirmed by LCMS. After completion of reaction, the reaction mixture was poured into ice cold water (10 ml). The resulting solid was filtered off, washed with water and dried under vacuum to obtain 2-(4-chloro-3-fluorophenoxy)-N-(4-(2-(2-(4-chlorophenoxy)acetyl)hydrazinecarbonyl)piperidin-1-yl)acetamide (0.090 g, 58.44% yield) as an off white solid. LCMS 513.3 [M+H]⁺.

### Step 3: Synthesis of 2-(4-chloro-3-fluorophenoxy)-N-(4-(5-((4-chlorophenoxy)methyl)-1,3,4-oxadiazol-2-yl)piperidin-1-yl)acetamide

To a stirred mixture of 2-(4-chloro-3-fluorophenoxy)-N-(4-(2-(2-(4-chlorophenoxy)acetyl)hydrazinecarbonyl)piperidin-1-yl)acetamide (0.090 g, 0.1754 mmol, 1.0 equiv), K₂CO₃ (0.121 g, 0.877 mmol, 5.0 equiv) & molecular sieves (0.090 g) in acetonitrile (10 mL) was added 4-toluenesulfonyl chloride (0.083 g, 0.4385 mmol, 2.5 equiv). The resulting reaction mixture was stirred at 100° C. overnight. The product formation was confirmed by LCMS. After completion of reaction, the reaction mixture was filtered and filtrate was diluted with EtOAc (20 mL). Organic layer was washed with water (2×10 mL) & brine (50 mL), dried over anhydrous Na₂SO₄ and concentrated. The crude product was purified by flash chromatography to obtain 2-(4-chloro-3-fluorophenoxy)-N-(4-(5-((4-chlorophenoxy)methyl)-1,3,4-oxadiazol-2-yl)piperidin-1-yl)acetamide (Compound 240-0.018 g, 20.93% Yield) as an off white solid. LCMS 495.5 [M+H]⁺. ¹H NMR (400 MHz, DMSO-d₆) δ 9.25 (s, 0.5H), 8.89 (s, 0.5H), 7.55-7.45 (m, 1H), 7.44-7.29 (m, 2H), 7.09 (d, J=8.8 Hz, 2H), 7.05-6.97 (m, 1H), 6.86-6.72 (m, 1H), 5.39 (s, 2H), 4.91 (s, 1H), 4.48 (s, 1H), 3.17 (d, J=5.3 Hz, 2H), 2.93 (br. s., 2H), 2.77 (br. s., 1H), 2.03 (br. s., 2H), 1.81 (d, J=11.4 Hz, 2H).

### Example 56

### Synthesis of trans-N-(5-chlorobenzo[d]oxazol-2-yl)-4-(5-(3-(cis-trifluoromethoxy)cyclobutyl)-1,3,4-oxadiazol-2-yl)cyclohexanecarboxamide

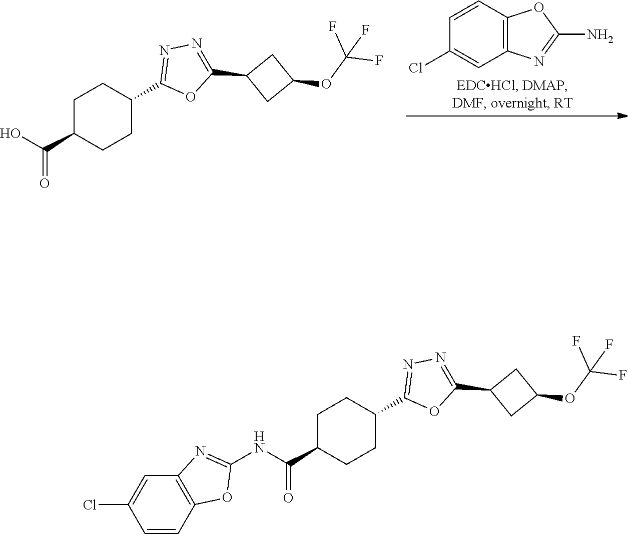

To a stirred mixture of trans-4-(5-(cis-3-(trifluoromethoxy)cyclobutyl)-1,3,4-oxadiazol-2-yl)cyclohexanecarboxylic acid (25 mg, 0.075 mmol, 1.0 equiv), EDCI.HCl (57 mg, 0.299 mmol, 4.0 equiv) & DMAP (37 mg, 0.299 mmol, 4.0 equiv) & in DCM (0.5 mL) was added 5-chlorobenzo[d]oxazol-2-amine (13 mg, 0.075 mmol, 1.0 equiv). The resulting reaction mixture was stirred at RT overnight. The product formation was confirmed by LCMS. After completion of reaction, the reaction mixture was concentrated under reduced pressure. The crude product was enriched by flash chromatography (0-5% MeOH in DCM as an eluent) which was further crystallized in methanol to obtain trans-N-(5-chlorobenzo[d]oxazol-2-yl)-4-(5-(3-(cis-trifluoromethoxy)cyclobutyl)-1,3,4-oxadiazol-2-yl)cyclohexanecarboxamide (Compound 241-6 mg, 16.7% yield) as a white solid. LCMS 531.5 [M+H]⁺. ¹H NMR (400 MHz, DMSO-d₆) δ 11.78 (br. s., 1H), 7.65 (d, J=9.6 Hz, 2H), 7.30 (dd, J=2.0, 8.6 Hz, 1H), 4.90 (td, J=7.5, 14.9 Hz, 1H), 3.55-3.37 (m, 2H), 2.96 (br. s., 1H), 2.89-2.76 (m, 2H), 2.67 (br. s., 2H), 2.15 (d, J=10.5 Hz, 2H), 2.00 (d, J=11.0 Hz, 2H), 1.69-1.42 (m, 4H).

### Example 57

### Synthesis of 2-(4-chloro-3-fluorophenoxy)-N-(4-(5-(cis-3-(trifluoromethoxy)cyclobutyl)-1,3,4-oxadiazol-2-yl)piperidin-1-yl)acetamide

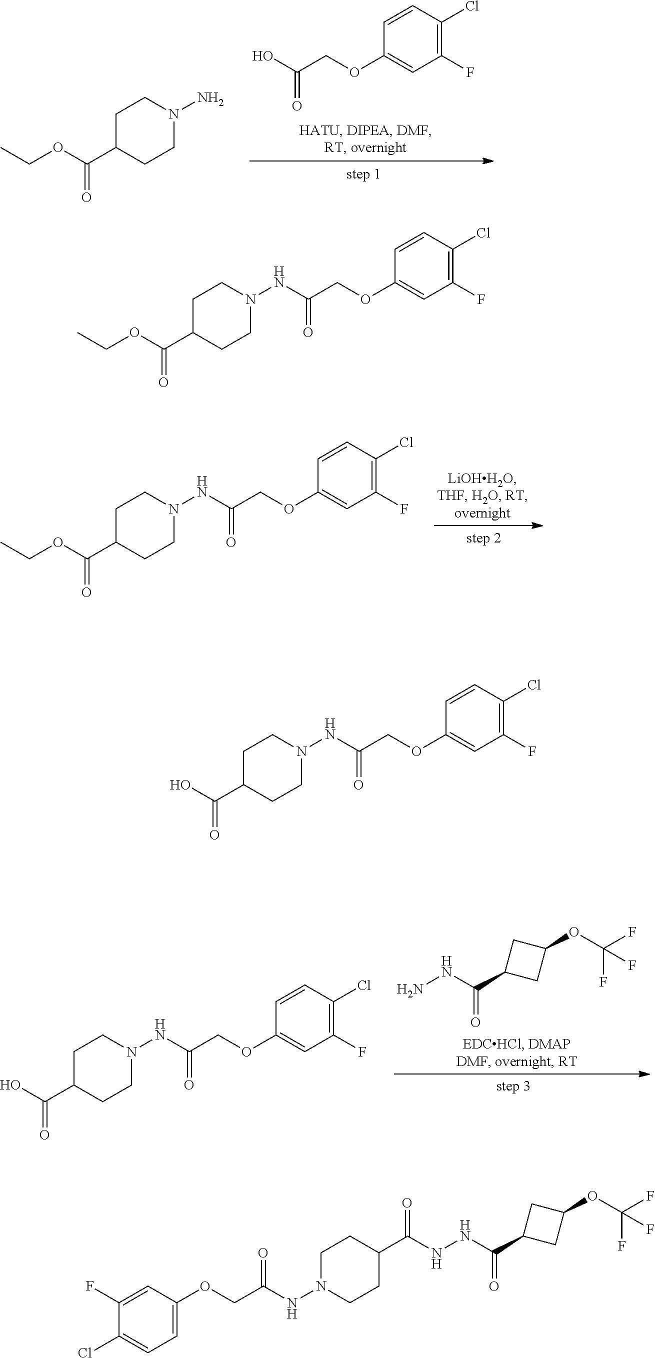

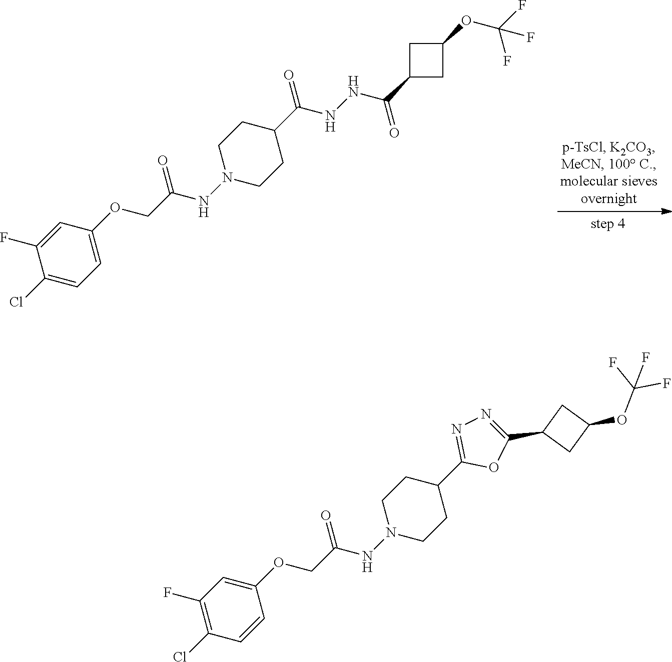 [SMILES: O=C(COc1ccc(Cl)c(F)c1)NN1CCC(C(=O)NNC(=O)[C@H]2C[C@@H](OC(F)(F)F)C2)CC1 ; O=C(COc1ccc(Cl)c(F)c1)NN1CCC(c2nnc([C@H]3C[C@@H](OC(F)(F)F)C3)o2)CC1]

### Step 1: Synthesis of ethyl 1-(2-(4-chloro-3-fluorophenoxy)acetamido)piperidine-4-carboxylate

To a stirred solution of ethyl 1-aminopiperidine-4-carboxylate (100 mg, 0.581 mmols, 1.0 equiv), 2-(4-chloro-3-fluorophenoxy)acetic acid (142 mg, 0.697 mmols, 1.0 equiv) and HATU (441 mg, 1.162 mmols, 2.0 equiv) in DMF (2 mL) was added DIPEA (0.2 mL, 1.162 mmols, 2.0 equiv). The resulting reaction mixture was stirred at RT overnight. Product formation was confirmed by LCMS. After completion of reaction, the reaction mixture was poured into ice cold water (10 ml). The resulting solid was filtered off and dried under vacuum. The crude product was enriched by flash chromatography (0-5% MeOH in DCM as an eluent) to obtain ethyl 1-(2-(4-chloro-3-fluorophenoxy)acetamido)piperidine-4-carboxylate (100 mg, 48% yield) as an off white solid. LCMS 359.2 [M+H]⁺.

### Step 2: Synthesis of 1-(2-(4-chloro-3-fluorophenoxy)acetamido)piperidine-4-carboxylic acid

To as stirred solution of ethyl 1-(2-(4-chloro-3-fluorophenoxy)acetamido)piperidine-4-carboxylate (200 mg, 0.558 mmols) in THF:Water (5:5 mL) was added LiOH.H₂O (28 mg, 0.669 mmols) at RT overnight. Product formation was confirmed by LCMS. The reaction mixture was acidified with 2 N HCl. The resulting solid was filtered off and dried under vacuum to obtain 1-(2-(4-chloro-3-fluorophenoxy)acetamido)piperidine-4-carboxylic acid (130 mg, 70% yield) as an off white solid. LCMS 331.4 [M+H]⁺.

### Step 3: Synthesis of 2-(4-chloro-3-fluorophenoxy)-N-(4-(2-(cis-3-(trifluoromethoxy)cyclobutanecarbonyl)hydrazinecarbonyl)piperidin-1-yl)acetamide

To a stirred mixture of 1-(2-(4-chloro-3-fluorophenoxy)acetamido)piperidine-4-carboxylic acid (100 mg, 0.303 mmol, 1.0 equiv), EDCI.HCl (175 mg, 0.909 mmol, 3 equiv) & DMAP (110 mg, 0.909 mmol, 3 equiv) & in DCM (10 mL) was added cis-3-(trifluoromethoxy)cyclobutanecarbohydrazide (60 mg, 0.303 mmol, 1.0 equiv). The resulting reaction mixture was stirred at RT overnight. The product formation was confirmed by LCMS. After completion of reaction, the reaction mixture was diluted with EtOAc (50 mL). Organic layer was washed with water (20 mL) and brine (20 mL), dried over anhydrous Na₂SO₄ & concentrated under reduced pressure to obtain 2-(4-chloro-3-fluorophenoxy)-N-(4-(2-(cis-3-(trifluoromethoxy)cyclobutanecarbonyl)hydrazinecarbonyl)piperidin-1-yl)acetamide (100 mg, 64.9% yield) as a white solid. LCMS 511.2 [M+H]⁺.

### Step 4: Synthesis of 2-(4-chloro-3-fluorophenoxy)-N-(4-(5-(cis-3-(trifluoromethoxy)cyclobutyl)-1,3,4-oxadiazol-2-yl)piperidin-1-yl)acetamide

To a stirred mixture of 2-(4-chloro-3-fluorophenoxy)-N-(4-(2-(cis-3-(trifluoromethoxy)cyclobutanecarbonyl)hydrazinecarbonyl)piperidin-1-yl)acetamide (200 mg, 0.39 mmol, 1.0 equiv), K₂CO₃ (250 mg, 1.81 mmol, 4.6 equiv) & molecular sieves (200 mg) in acetonitrile (50 mL) was added 4-toluenesulfonyl chloride (200 mg, 1.05 mmol, 2.7 equiv). The resulting reaction mixture was stirred at 100° C. overnight. Reaction was monitored by LCMS and TLC. Reaction mixture was filtered, concentrated under vacuum, and the residue obtained diluted with EtOAc (50 mL). Organic extracts were washed with water (20 mL) & brine (20 mL), dried over anhydrous Na₂SO₄ and concentrated. The crude product was purified by reverse phase HPLC to obtain to obtain 2-(4-chloro-3-fluorophenoxy)-N-(4-(5-(cis-3-(trifluoromethoxy)cyclobutyl)-1,3,4-oxadiazol-2-yl)piperidin-1-yl)acetamide (Compound 242-60 mg, 31% yield) as a white solid. LCMS 493.1 [M+H]⁺. ¹H NMR (400 MHz, DMSO-d₆) δ 9.24 (s, 0.5H), 8.88 (s, 0.5H), 7.53-7.33 (m, 1H), 7.11-6.89 (m, 1H), 6.88-6.61 (m, 1H), 4.99-4.81 (m, 2H), 4.48 (s, 1H), 3.54-3.36 (m, 1H), 3.14 (br. s., 1H), 2.95 (d, J=10.5 Hz, 2H), 2.89-2.70 (m, 4H), 2.67 (br. s., 1H), 2.14-1.92 (m, 3H), 1.89-1.67 (m, 2H).

### Example 58

### Synthesis of trans-2-(4-chloro-3-fluorophenoxy)-N-(4-(5-(cis-3-(trifluoromethyl)cyclobutyl)-1,3,4-oxadiazol-2-yl)cyclohexyl)acetamide

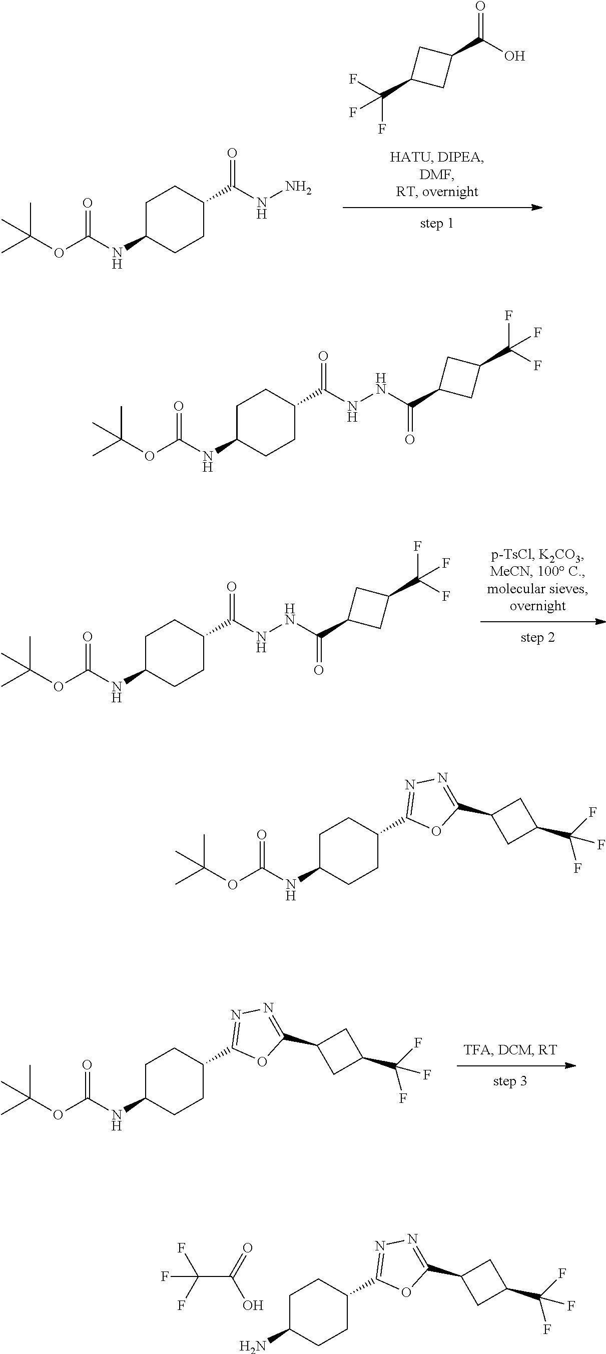

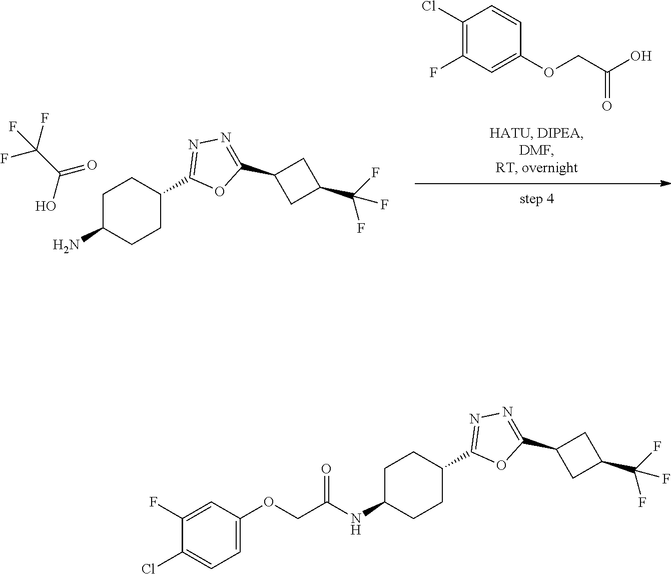

### Step 1: Synthesis of trans-tert-butyl (4-(2-(cis-3-(trifluoromethyl)cyclobutanecarbonyl)hydrazinecarbonyl)cyclohexyl)carbamate

To a stirred mixture of trans-tert-butyl 4-(hydrazinecarbonyl)cyclohexylcarbamate (200 mg, 0.778 mmol, 1.0 equiv) cis-3-(trifluoromethyl)cyclobutanecarboxylic acid (130 mg, 0.778 mmol, 1.0 equiv) & HATU (443 mg, 1.17 mmol, 1.0 equiv) in DMF (5 mL) was added DIPEA (0.56 mL, 3.112 mmol, 4.0 equiv). The resulting reaction mixture was stirred at RT overnight. The product formation was confirmed by LCMS. After completion of reaction, the reaction mixture was poured into ice cold water (100 ml). The resulting solid was filtered off and dried under vacuum to obtain trans-tert-butyl (4-(2-(cis-3-(trifluoromethyl)cyclobutanecarbonyl)hydrazinecarbonyl)cyclohexyl)carbamate (240 mg, 75.9% yield) as an off white solid. LCMS 408.3 [M+H]⁺. ¹H NMR (400 MHz, DMSO-d₆) δ 9.72 (s, 1H) 9.64 (s, 1H) 6.72 (d, J=7.89 Hz, 1H) 3.07-3.27 (m, 2H) 3.02 (d, J=8.77 Hz, 1H) 2.12-2.27 (m, 5H) 2.06 (br. s., 1H) 1.80 (d, J=10.52 Hz, 3H) 1.71 (d, J=13.15 Hz, 2H) 1.29-1.48 (m, 9H) 1.13 (d, J=13.15 Hz, 2H).

### Step 2: Synthesis of trans-tert-butyl (4-(5-(cis-3-(trifluoromethyl)cyclobutyl)-1,3,4-oxadiazol-2-yl)cyclohexyl)carbamate

To a stirred mixture of trans-tert-butyl (4-(2-(cis-3-(trifluoromethyl)cyclobutanecarbonyl)hydrazinecarbonyl)cyclohexyl)carbamate (240 mg, 0.589 mmol, 1.0 equiv), K₂CO₃ (406 mg, 2.95 mmol, 5.0 equiv) & molecular sieves (500 mg) in acetonitrile (20 mL) was added 4-toluenesulfonyl chloride (280 mg, 1.47 mmol, 2.5 equiv) and the resultant reaction mixture was stirred at 100° C. overnight. The product formation was confirmed by LCMS and TLC. After completion of reaction, the reaction mixture was filtered off and the filtrate was diluted with EtOAc (100 mL). Organic layer was washed with water (50 mL) & brine (50 mL), dried over anhydrous Na₂SO₄ and concentrated under reduced pressure to obtain trans-tert-butyl (4-(5-(cis-3-(trifluoromethyl)cyclobutyl)-1,3,4-oxadiazol-2-yl)cyclohexyl)carbamate (210 mg, 91.7% yield) as an off white solid. LCMS 390.3 [M+H]⁺. ¹H NMR (400 MHz, DMSO-d₆) δ 6.81 (d, J=7.89 Hz, 1H) 3.75 (dt, J=17.87, 8.82 Hz, 1H) 3.16-3.30 (m, 2H) 2.80 (t, J=11.84 Hz, 1H) 2.53-2.65 (m, 2H) 2.25-2.43 (m, 1H) 1.93-2.09 (m, 2H) 1.86 (d, J=10.09 Hz, 2H) 1.45-1.57 (m, 2H) 1.38 (s, 9H) 1.31 (br. s., 1H) 1.06-1.29 (m, 2H).

### Step 3: Synthesis of trans-4-(5-(cis-3-(trifluoromethyl)cyclobutyl)-1,3,4-oxadiazol-2-yl)cyclohexanamine 2,2,2-trifluoroacetate

To a stirred solution of trans-tert-butyl (4-(5-(cis-3-(trifluoromethyl)cyclobutyl)-1,3,4-oxadiazol-2-yl)cyclohexyl)carbamate (210 mg, 0.54 mmol, 1.0 equiv) in DCM (7 mL) was added trifluoroacetic acid (0.3 mL) and the resulting reaction mixture was stirred at RT overnight under nitrogen atmosphere. The product formation was confirmed by LCMS. After completion of reaction, the reaction mixture was concentrated under reduced pressure. The crude product was crystallized in diethyl ether and dried under vacuum to trans-4-(5-(cis-3-(trifluoromethyl)cyclobutyl)-1,3,4-oxadiazol-2-yl)cyclohexanamine 2,2,2-trifluoroacetate (160 mg, 73.7% yield) as an off white solid. LCMS 290.3 [M+H]⁺.

### Step 4: Synthesis of trans-2-(4-chloro-3-fluorophenoxy)-N-(4-(5-(cis-3-(trifluoromethyl)cyclobutyl)-1,3,4-oxadiazol-2-yl)cyclohexyl)acetamide

To a stirred solution of trans-4-(5-(cis-3-(trifluoromethyl)cyclobutyl)-1,3,4-oxadiazol-2-yl)cyclohexanamine 2,2,2-trifluoroacetate (100 mg, 0.248 mmol, 1.0 equiv), 2-(4-chloro-3-fluorophenoxy)acetic acid (51 mg, 0.248 mmol, 1.0 equiv) & HATU (141 mg, 0.372 mmol, 1.5 equiv) in DMF (5 mL) was added DIPEA (0.2 mL, 0.99 mmol, 4.0 equiv). The resulting reaction mixture was stirred at RT overnight. The product formation was confirmed by LCMS. After completion of reaction, the reaction mixture was diluted with water (50 mL) and extracted with EtOAc (2×50 mL). Organic extracts were washed with water (4×50 mL) & brine (50 mL), dried over anhydrous Na₂SO₄ and concentrated under reduced pressure. The crude product was purified by reversed phase HPLC to obtain trans-2-(4-chloro-3-fluorophenoxy)-N-(4-(5-(cis-3-(trifluoromethyl)cyclobutyl)-1,3,4-oxadiazol-2-yl)cyclohexyl)acetamide (Compound 243-60 mg, 51.3% yield) as a white solid. LCMS 476.6 [M+H]⁺. ¹H NMR (400 MHz, DMSO-d₆) δ 8.03 (d, J=7.9 Hz, 1H), 7.50 (t, J=9.0 Hz, 1H), 7.07 (dd, J=2.6, 11.4 Hz, 1H), 6.85 (d, J=11.0 Hz, 1H), 4.51 (s, 2H), 3.87-3.74 (m, 1H), 3.71-3.58 (m, 1H), 3.29-3.22 (m, 1H), 2.87 (t, J=12.1 Hz, 1H), 2.65-2.52 (m, 2H), 2.41-2.28 (m, 2H), 2.08 (d, J=9.6 Hz, 2H), 1.86 (d, J=10.5 Hz, 2H), 1.63-1.48 (m, 2H), 1.44-1.33 (m, 2H).

### Example 59

### Synthesis of N-(trans-4-(5-(4-chloro-3-fluorobenzyl)-1,3,4-oxadiazol-2-yl)cyclohexyl)-2-(4-chloro-3-fluorophenoxy)acetamide

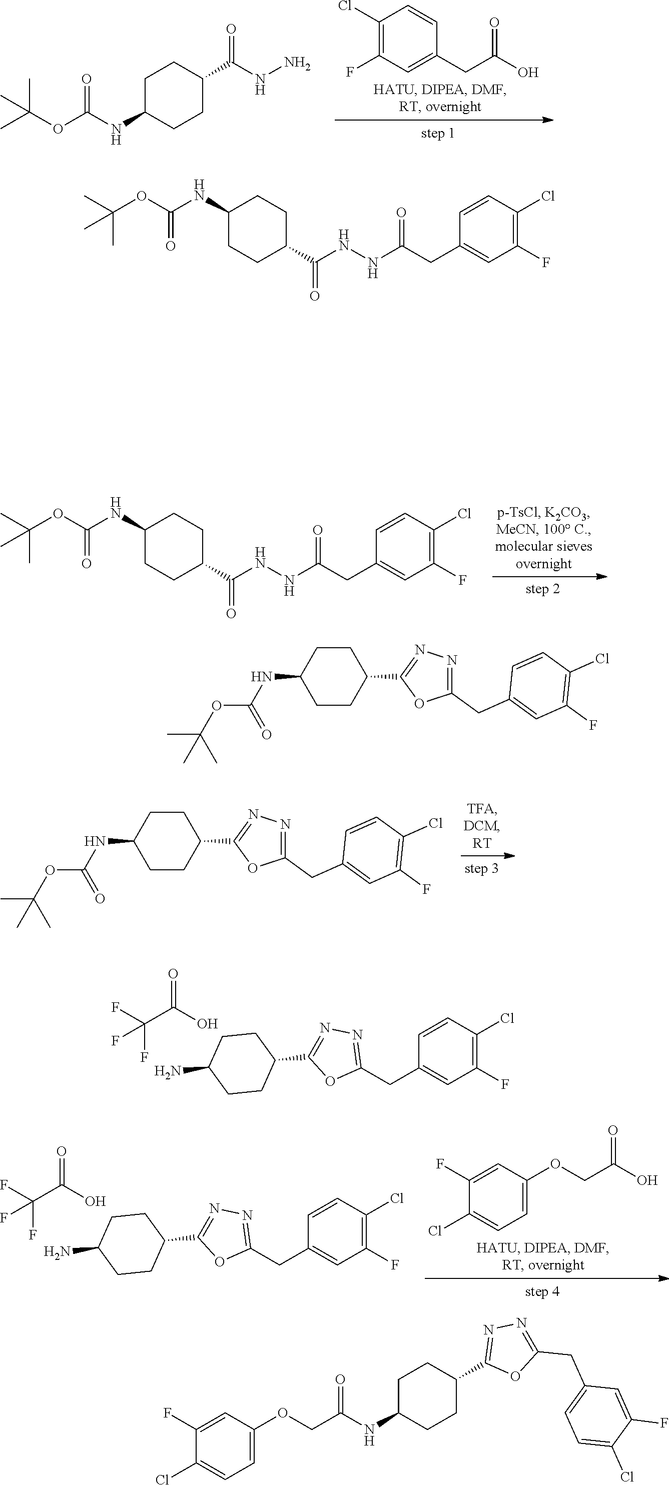

### Step 1: Synthesis of trans-tert-butyl (4-(2-(2-(4-chloro-3-fluorophenyl)acetyl)hydrazinecarbonyl)cyclohexyl)carbamate

To a stirred solution of trans-tert-butyl 4-(hydrazinecarbonyl)cyclohexylcarbamate (200 mg, 1.06 mmol, 1.0 equiv), 2-(4-chloro-3-fluorophenyl)acetic acid (272 mg, 1.06 mmol, 1.0 equiv) and HATU (604 mg, 1.59 mmol, 1.5 equiv) in DMF (10 mL) was added DIPEA (0.8 mL, 4.24 mmol, 4.0 equiv). The resulting reaction mixture was stirred at RT overnight. Product formation was confirmed by LCMS. After completion of reaction, the reaction mixture was poured into ice cold water (100 ml). The resulting white solid was filtered off and dried under vacuum to obtain trans-tert-butyl (4-(2-(2-(4-chloro-3-fluorophenyl)acetyl)hydrazinecarbonyl)cyclohexyl)carbamate (400 mg, 81.76% yield) as an off white solid. ¹H NMR (400 MHz, DMSO-d₆) δ 10.02 (s, 1H), 9.72 (s, 1H), 7.52 (t, J=8.1 Hz, 1H), 7.34 (dd, J=1.5, 10.3 Hz, 1H), 7.15 (d, J=9.6 Hz, 1H), 6.71 (d, J=7.5 Hz, 1H), 3.57-3.46 (m, 2H), 3.21-3.08 (m, 1H), 2.07 (t, J=11.8 Hz, 1H), 1.79 (d, J=10.1 Hz, 2H), 1.71 (d, J=11.4 Hz, 2H), 1.42 (br. s., 1H), 1.40-1.22 (m, 11H), 1.20-0.98 (m, 2H).

### Step 2: Synthesis of trans-tert-butyl (4-(5-(4-chloro-3-fluorobenzyl)-1,3,4-oxadiazol-2-yl)cyclohexyl)carbamate

To a stirred solution of trans-tert-butyl (4-(2-(2-(4-chloro-3-fluorophenyl)acetyl)hydrazinecarbonyl)cyclohexyl)carbamate (400 mg, 0.936 mmol, 1.0 equiv), K₂CO₃ (645 mg, 4.68 mmol, 5.0 equiv) and molecular sieves (1000 mg) in acetonitrile (25 mL) was added 4-toluenesulfonyl chloride (444 mg, 2.34 mmol, 2.5 equiv) and the resulting reaction mixture was heated at 100° C. overnight. Product formation was confirmed by LCMS and TLC. After completion of reaction, the reaction mixture was filtered through Celite®. The filtrate was dissolved in EtOAc (100 mL). The organic layer was washed with water (50 mL) and brine (50 mL), dried over anhydrous Na₂SO₄ and concentrated to obtain trans-tert-butyl (4-(5-(4-chloro-3-fluorobenzyl)-1,3,4-oxadiazol-2-yl)cyclohexyl)carbamate (350 mg, 91.38% yield) as an off white solid. LCMS 410.3 [M+H]⁺. ¹H NMR (400 MHz, DMSO-d₆) δ 7.64-7.49 (m, 1H), 7.45-7.33 (m, 1H), 7.27-7.05 (m, 1H), 6.80 (d, J=7.0 Hz, 1H), 4.28 (s, 2H), 3.22 (br. s., 1H), 2.85-2.72 (m, 1H), 2.06-1.95 (m, 2H), 1.85 (d, J=11.4 Hz, 2H), 1.58-1.45 (m, 2H), 1.37 (s, 9H), 1.28-1.10 (m, 2H).

### Step 3: Synthesis of trans-4-(5-(4-chloro-3-fluorobenzyl)-1,3,4-oxadiazol-2-yl)cyclohexanamine 2,2,2-trifluoroacetate

To a stirred solution of trans-tert-butyl (4-(5-(4-chloro-3-fluorobenzyl)-1,3,4-oxadiazol-2-yl)cyclohexyl)carbamate (150 mg, 0.367 mmol, 1.0 equiv) in DCM (5 mL) was added trifluoroacetic acid (0.05 mL) and the resulting reaction mixture was stirred at RT overnight under nitrogen atmosphere. Product formation was confirmed by LCMS. After completion of reaction, the reaction mixture was concentrated under reduced pressure. The crude product was crystallized in diethyl ether, dried under vacuum to obtain trans-4-(5-(4-chloro-3-fluorobenzyl)-1,3,4-oxadiazol-2-yl)cyclohexanamine 2,2,2-trifluoroacetate (150 mg, 96.8% yield) as a brown solid. LCMS 310.5 [M+H]⁺.

### Step 4: Synthesis of N-(trans-4-(5-(4-chloro-3-fluorobenzyl)-1,3,4-oxadiazol-2-yl)cyclohexyl)-2-(4-chloro-3-fluorophenoxy)acetamide

To a stirred solution of trans-4-(5-(4-chloro-3-fluorobenzyl)-1,3,4-oxadiazol-2-yl)cyclohexanamine 2,2,2-trifluoroacetate (100 mg, 0.236 mmol, 1.0 equiv), 2-(4-chloro-3-fluorophenoxy)acetic acid (48 mg, 0.236 mmol, 1 equiv) and HATU (134 mg, 0.35 mmol, 1.5 equiv) in DMF (5 mL) was added DIPEA (0.2 mL, 0.944 mmol, 4.0 equiv) and the resulting reaction mixture was stirred at RT overnight. Product formation was confirmed by LCMS and TLC. After completion of reaction, the reaction mixture was poured into ice cold water (100 ml). The resulting solid was filtered off and dried under vacuum to obtain crude product which was purified by reversed phase HPLC to obtain N-(trans-4-(5-(4-chloro-3-fluorobenzyl)-1,3,4-oxadiazol-2-yl)cyclohexyl)-2-(4-chloro-3-fluorophenoxy)acetamide (Compound 244-60 mg, 51.4% yield) as an off white solid. LCMS 478.5 [M+H]⁺. ¹H NMR (400 MHz, DMSO-d₆) δ 8.04 (d, J=7.9 Hz, 1H), 7.67-7.56 (m, 1H), 7.56-7.46 (m, 1H), 7.42 (dd, J=1.8, 10.5 Hz, 1H), 7.20 (d, J=8.3 Hz, 1H), 7.06 (dd, J=2.6, 11.4 Hz, 1H), 6.85 (dd, J=2.0, 8.6 Hz, 1H), 4.51 (s, 2H), 4.29 (s, 2H), 3.68-3.55 (m, 1H), 2.86 (t, J=11.8 Hz, 1H), 2.06 (d, J=11.8 Hz, 2H), 1.85 (d, J=9.6 Hz, 2H), 1.64-1.45 (m, 2H), 1.45-1.28 (m, 2H).

### Example 60

### Chiral Separation of trans-(S)-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide and trans-(R)-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide

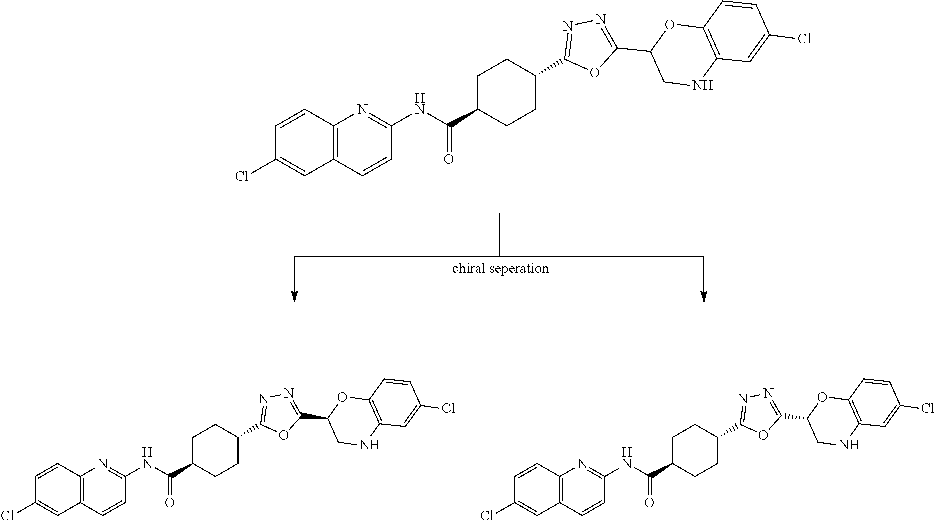 [SMILES: O=C(Nc1ccc2cc(Cl)ccc2n1)[C@H]1CC[C@H](c2nnc(C3CNc4cc(Cl)ccc4O3)o2)CC1 ; O=C(Nc1ccc2cc(Cl)ccc2n1)[C@H]1CC[C@H](c2nnc([C@@H]3CNc4cc(Cl)ccc4O3)o2)CC1 ; O=C(Nc1ccc2cc(Cl)ccc2n1)[C@H]1CC[C@H](c2nnc([C@H]3CNc4cc(Cl)ccc4O3)o2)CC1]

The enantiomers, trans-(S)-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide (Compound 245—[α]_{D}²⁰=NA; (elution time: 10.20 min) and trans-(R)-4-(5-(6-chloro-3,4-dihydro-2H-benzo[b][1,4]oxazin-2-yl)-1,3,4-oxadiazol-2-yl)-N-(6-chloroquinolin-2-yl)cyclohexanecarboxamide (Compound 246 [α]_{D}²⁰=NA); elution time: 14.7 min), were separated by chiral SFC (Chiralcel-OX-H, 250×21 mm, 5p). Isocratic program with analytical grade liquid carbon dioxide and 0.1% liq. Ammonia in HPLC grade MeOH. LCMS; 424.5 [M+H]⁺1H NMR (400 MHz, DMSO-d₆) δ 10.85 (s, 1H) 8.24-8.41 (m, 2H) 8.05 (d, J=2.19 Hz, 1H) 7.80 (d, J=9.21 Hz, 1H) 7.68-7.75 (m, 1H) 6.79 (d, J=8.33 Hz, 1H) 6.65 (d, J=2.19 Hz, 1H) 6.52 (dd, J=8.55, 2.41 Hz, 1H) 6.37 (br. s., 1H) 5.51 (dd, J=6.14, 3.07 Hz, 1H) 3.61-3.76 (m, 1H) 3.54-3.61 (m, 1H) 3.01 (t, J=11.18 Hz, 1H) 2.65 (d, J=16.66 Hz, 1H) 2.14 (d, J=10.96 Hz, 2H) 1.99 (d, J=11.84 Hz, 2H) 1.42-1.72 (m, 4H).

### Example 61

### Synthesis of trans-5-chloro-N-(4-(5-(4-chlorobenzyl)-1,3,4-oxadiazol-2-yl)cyclohexyl)benzofuran-2-carboxamide

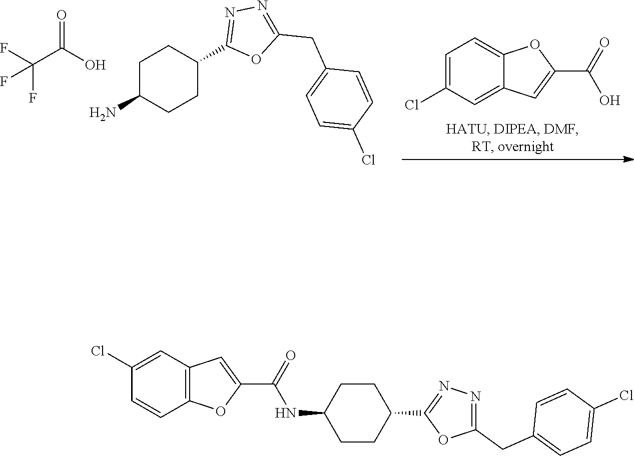

To a stirred mixture of trans-4-(5-(4-chlorobenzyl)-1,3,4-oxadiazol-2-yl)cyclohexanamine 2,2,2-trifluoroacetate (0.100 g, 0.2469 mmol, 1.0 equiv), 5-chlorobenzofuran-2-carboxylic acid (0.048 g, 0.2469 mmol, 1.0 equiv) & HATU (0.140 g, 0.3703 mmol, 1.5 equiv) in DMF (3 mL) was added DIPEA (0.127 g, 0.9876 mmol, 4.0 equiv). The resulting reaction mixture was stirred at RT for overnight. The product formation was confirmed by LCMS. After completion of reaction, the reaction mixture was quenched with ice cold water (20 ml). The resulting solid was filtered off and dried under vacuum and further purified by flash chromatography (0-5% MeOH in DCM as an eluent) to obtain trans-5-chloro-N-(4-(5-(4-chlorobenzyl)-1,3,4-oxadiazol-2-yl)cyclohexyl)benzofuran-2-carboxamide (Compound 247-6 mg, 5.25% Yield) as an off white solid. LCMS 470.5 [M+H]⁺. ¹H NMR (400 MHz, DMSO-d₆) δ 8.66 (d, J=8.3 Hz, 1H), 7.87 (s, 1H), 7.69 (d, J=9.2 Hz, 1H), 7.53 (s, 1H), 7.48 (d, J=8.8 Hz, 1H), 7.42 (d, J=8.3 Hz, 2H), 7.35 (d, J=8.3 Hz, 2H), 4.26 (s, 2H), 3.82 (br. s., 1H), 2.87 (br. s., 1H), 2.09 (d, J=9.6 Hz, 2H), 1.94 (br. s., 2H), 1.64-1.40 (m, 4H).

### Example 62

### Synthesis of trans-N-(6-chloroquinolin-2-yl)-4-(5-(cis-3-(trifluoromethyl)cyclobutyl)-1,3,4-oxadiazol-2-yl)cyclohexanecarboxamide

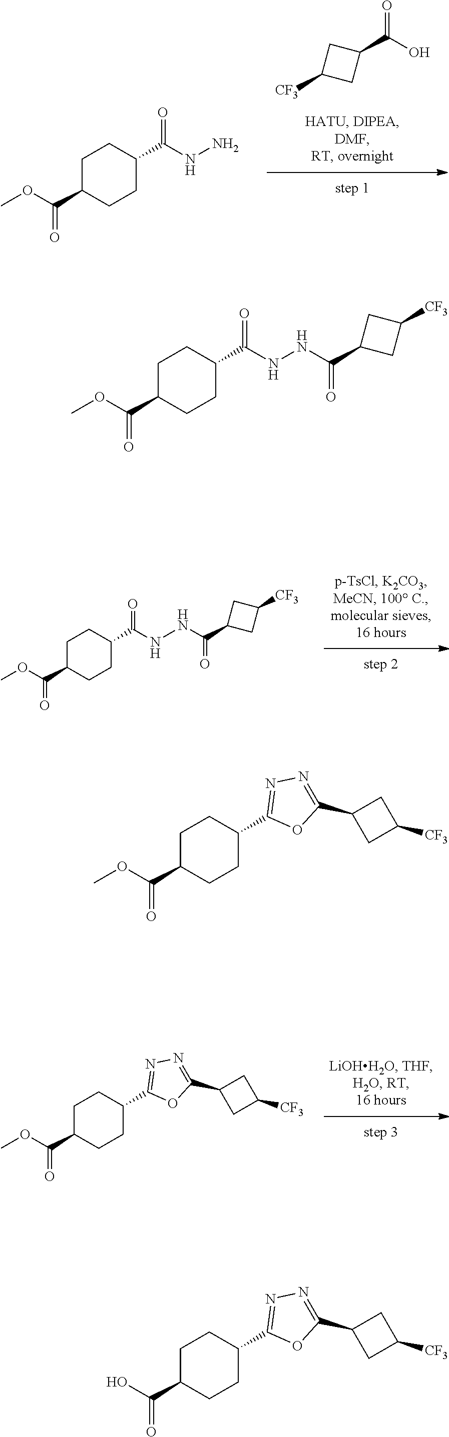

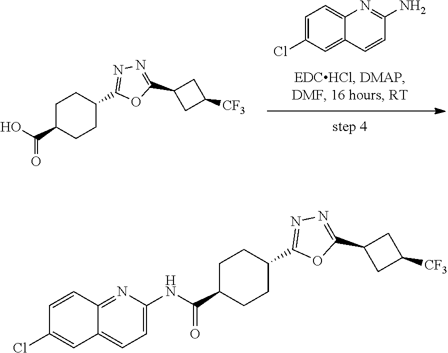 [SMILES: Nc1ccc2cc(Cl)ccc2n1 ; O=C(O)[C@H]1CC[C@H](c2nnc([C@H]3C[C@@H](C(F)(F)F)C3)o2)CC1 ; O=C(Nc1ccc2cc(Cl)ccc2n1)[C@H]1CC[C@H](c2nnc([C@H]3C[C@@H](C(F)(F)F)C3)o2)CC1]

### Step 1: Synthesis of trans-methyl 4-(2-(cis-3-(trifluoromethyl)cyclobutanecarbonyl)hydrazinecarbonyl)cyclohexanecarboxylate

To a stirred mixture of trans-methyl 4-(hydrazinecarbonyl)cyclohexanecarboxylate (0.357 g, 1.784 mmol, 1.5 equiv), cis-3-(trifluoromethyl)cyclobutanecarboxylic acid (0.200 g, 1.189 mmol, 1.0 equiv) & HATU (0.678 g, 1.784 mmol, 1.5 equiv) in DMF (4 mL) was added DIPEA (0.82 mL, 4.756 mmol, 4.0 equiv). The resulting reaction mixture was stirred at RT overnight. The product formation was confirmed by LCMS. After completion of reaction, the reaction mixture was poured into ice cold water (30 ml). The resulting solid was filtered off, washed with water and dried under vacuum to obtain trans-methyl 4-(2-(cis-3-(trifluoromethyl)cyclobutanecarbonyl)hydrazinecarbonyl)cyclohexanecarboxylate (0.200 g, 48% yield) as an off white solid. LCMS 351.3 [M+H]⁺.

### Step 2: Synthesis of trans-methyl 4-(5-(cis-3-(trifluoromethyl)cyclobutyl)-1,3,4-oxadiazol-2-yl)cyclohexanecarboxylate

To a stirred solution of trans-methyl 4-(2-(cis-3-(trifluoromethyl)cyclobutanecarbonyl)hydrazinecarbonyl)cyclohexanecarboxylate (0.240 g, 0.6850 mmol, 1.0 equiv), K₂CO₃ (0.472 g, 3.425 mmol, 5.0 equiv) and molecular sieves (0.240 g) in acetonitrile (30 mL) was added 4-toluenesulfonyl chloride (0.325 g, 1.712 mmol, 2.5 equiv) and the resulting reaction mixture was stirred at 100° C. overnight. Product formation was confirmed by LCMS and TLC. After completion of reaction, the reaction mixture filtered through Celite® and filtrate was diluted with EtOAc (50 mL). Organic layer was washed with water (2×20 mL), dried over anhydrous Na₂SO₄ and concentrated. The crude product was crystallized in hexane to obtain trans-methyl 4-(5-(cis-3-(trifluoromethyl)cyclobutyl)-1,3,4-oxadiazol-2-yl)cyclohexanecarboxylate (0.195 g, 85.90% yield) as an off white solid. LCMS 333.3 [M+H]⁺.

### Step 3: Synthesis of trans-4-(5-(cis-3-(trifluoromethyl)cyclobutyl)-1,3,4-oxadiazol-2-yl)cyclohexanecarboxylic acid

To a stirred solution of trans-methyl 4-(5-(cis-3-(trifluoromethyl)cyclobutyl)-1,3,4-oxadiazol-2-yl)cyclohexanecarboxylate (0.195 g, 0.5867 mmol, 1.0 equiv) in THF (5 mL) & water (5 mL) was added LiOH.H₂O (0.074 g 1.760 mmol, 3.0 equiv) and the resulting reaction mixture was stirred at RT for overnight. Product formation was confirmed by LCMS. After completion of reaction, the reaction mixture was concentrated under reduced pressure. The aqueous phase was washed with ethyl acetate (2×5 ml) and acidified with 1 N HCl up to PH˜2. The resulting precipitate was filtered off and washed with water (2×5 mL), hexane (2×5 mL) and dried under vacuum to obtain trans-4-(5-(cis-3-(trifluoromethyl)cyclobutyl)-1,3,4-oxadiazol-2-yl)cyclohexanecarboxylic acid (0.070 g, 37.63% yield) as an off white solid. LCMS 319.4 [M+H]⁺.

### Step 4: Synthesis of trans-N-(6-chloroquinolin-2-yl)-4-(5-(cis-3-(trifluoromethyl)cyclobutyl)-1,3,4-oxadiazol-2-yl)cyclohexanecarboxamide

To a stirred solution of trans-4-(5-(cis-3-(trifluoromethyl)cyclobutyl)-1,3,4-oxadiazol-2-yl)cyclohexanecarboxylic acid (0.070 g, 0.2199 mmol, 1.0 equiv), EDCI.HCl (0.168 g, 0.8794 mmol, 4.0 equiv) & DMAP (0.107 g, 0.8794 mmol, 4.0 equiv) in DCM (5 mL) was added 6-chloroquinolin-2-amine (0.039 g, 0.2199 mmol, 1.0 equiv). The resulting reaction mixture was stirred at RT for overnight. The product formation was confirmed by LCMS. After completion of reaction, the reaction mixture was quenched with ice cold water (10 mL) and extracted with EtOAc (20 mL). Organic layer was washed with saturated solution of citric acid (10 mL), dried over anhydrous Na₂SO₄ and concentrated. The crude product was purified by flash chromatography (0-5% MeOH in DCM as an eluent) to obtain trans-N-(6-chloroquinolin-2-yl)-4-(5-(cis-3-(trifluoromethyl)cyclobutyl)-1,3,4-oxadiazol-2-yl)cyclohexanecarboxamide (Compound 248-19.70 mg, 18.76% yield) as a white solid. LCMS 479.5 [M+H]⁺. ¹H NMR (400 MHz, DMSO-d₆) δ 10.84 (s, 1H), 8.46-8.23 (m, 2H), 8.05 (d, J=2.2 Hz, 1H), 7.80 (d, J=8.8 Hz, 1H), 7.71 (dd, J=2.2, 9.2 Hz, 1H), 3.83-3.70 (m, 1H), 2.95 (t, J=11.6 Hz, 1H), 2.73-2.54 (m, 4H), 2.42-2.27 (m, 2H), 2.14 (d, J=13.2 Hz, 2H), 1.99 (d, J=11.8 Hz, 2H), 1.71-1.42 (m, 4H).

### Example 63

### Synthesis of cis-4-(1-(4-chlorophenyl)-1H-pyrazol-4-yl)-N-(6-chloroquinolin-2-yl)cyclohexanecarboxamide and trans-4-(1-(4-chlorophenyl)-1H-pyrazol-4-yl)-N-(6-chloroquinolin-2-yl)cyclohexanecarboxamide

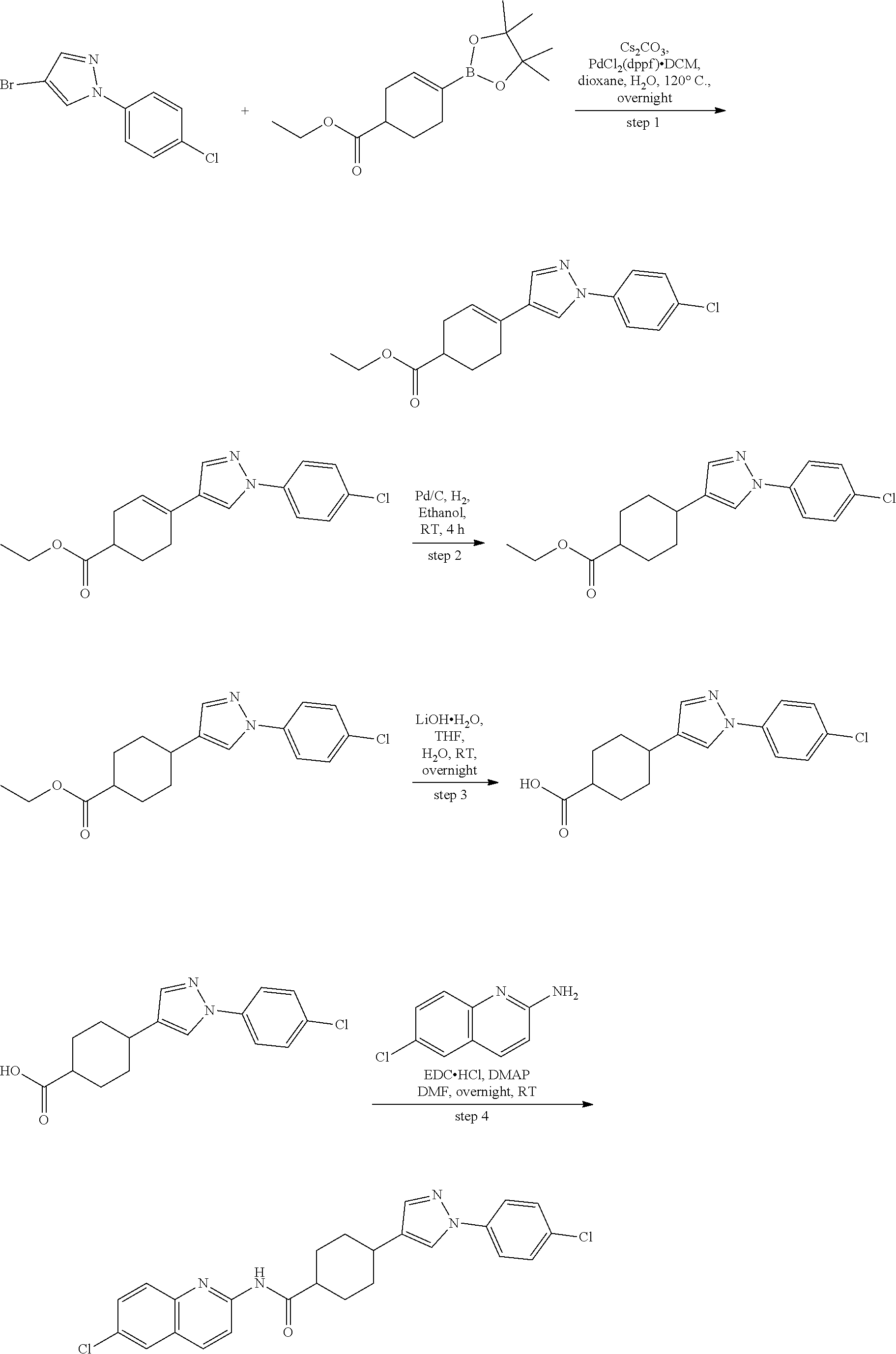

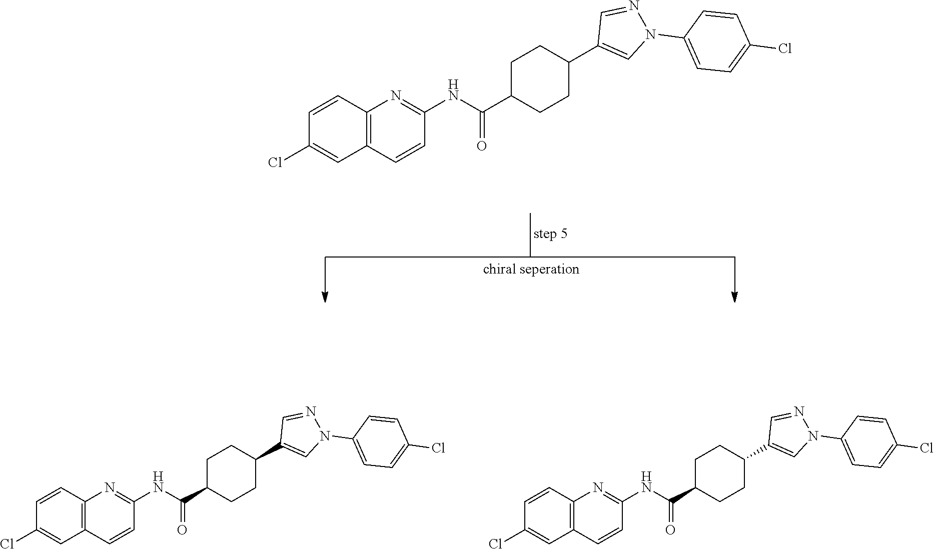 [SMILES: O=C(Nc1ccc2cc(Cl)ccc2n1)C1CCC(c2cnn(-c3ccc(Cl)cc3)c2)CC1 ; O=C(Nc1ccc2cc(Cl)ccc2n1)[C@H]1CC[C@@H](c2cnn(-c3ccc(Cl)cc3)c2)CC1 ; O=C(Nc1ccc2cc(Cl)ccc2n1)[C@H]1CC[C@H](c2cnn(-c3ccc(Cl)cc3)c2)CC1]

### Step 1: Synthesis of ethyl 4-(1-(4-chlorophenyl)-1H-pyrazol-4-yl)cyclohex-3-enecarboxylate

To a stirred mixture of ethyl 4-(4,4,5,5-tetramethyl-1,3,2-dioxaborolan-2-yl)cyclohex-3-enecarboxylate (100 mg, 0.351 mmol, 1.0 equiv), 4-bromo-1-(4-chlorophenyl)-1H-pyrazole (90 mg, 0.351 mmol, 1.0 equiv) & Cs₂CO₃ (228 mg, 0.702 mmol, 2.0 equiv) in dioxane (5 mL) and water (0.5 mL) was added PdCl₂ (dppf).DCM (28 mg, 0.351 mmol, 0.1 equiv). The resulting reaction mixture was refluxed at 120° C. overnight. The product formation was confirmed by LCMS and TLC. After completion of reaction, the reaction mixture was filtered through Celite® and washed with ethyl acetate (2×20 mL). Organic layer was concentrated and the crude product was purified by flash chromatography (20% ethyl acetate in hexane as an eluent) to obtain ethyl 4-(1-(4-chlorophenyl)-1H-pyrazol-4-yl)cyclohex-3-enecarboxylate (57 mg, 49.2% yield) as an off white solid. LCMS 331.2 [M+H]⁺. ¹H NMR (400 MHz, DMSO-d₆) δ 8.55 (s, 1H), 7.93 (s, 1H), 7.90-7.73 (m, J=8.8 Hz, 2H), 7.61-7.40 (m, J=8.8 Hz, 2H), 6.14 (br. s., 1H), 4.18-3.95 (m, 2H), 2.59 (br. s., 2H), 2.41 (br. s., 1H), 2.35 (d, J=14.5 Hz, 4H), 2.05 (d, J=11.8 Hz, 1H), 1.70 (br. s., 1H), 1.28-1.10 (m, 3H).

### Step 2: Synthesis of ethyl 4-(1-(4-chlorophenyl)-1H-pyrazol-4-yl)cyclohexanecarboxylate

To a stirred solution of ethyl 4-(1-(4-chlorophenyl)-1H-pyrazol-4-yl)cyclohex-3-enecarboxylate (150 mg, 0.455 mmol, 1.0 equiv) in ethanol (50 mL) was added Pd/C (20 mg, 10% Pd on carbon) under N₂. The reaction mixture was degassed under vacuum and purged with H₂ gas at RT. The product formation was confirmed by LCMS. After completion of reaction the reaction mixture was filtered through Celite® and concentrated to obtain ethyl 4-(1-(4-chlorophenyl)-1H-pyrazol-4-yl)cyclohexanecarboxylate (158 mg, 98% yield) as an off white solid. LCMS 333.3 [M+H]⁺.

### Step 3: Synthesis of 4-(1-(4-chlorophenyl)-1H-pyrazol-4-yl)cyclohexanecarboxylic acid

To a solution of ethyl 4-(1-(4-chlorophenyl)-1H-pyrazol-4-yl)cyclohexanecarboxylate (158 mg, 0.474 mmol, 1.0 equiv) in THE (4 mL) was added LiOH.H₂O (60 mg, 1.423 mmol, 3.0 equiv) in water (4 ml). The resulting mixture was stirred at RT overnight. The product formation was confirmed by LCMS. After completion of reaction, the reaction mixture was concentrated under reduced pressure. The residue was dissolved in water (4 mL). The aqueous layer was acidified by 6 M HCl (pH˜3). The resulting solid was filtered off and dried under vacuum to obtain 4-(1-(4-chlorophenyl)-1H-pyrazol-4-yl)cyclohexanecarboxylic acid (140 mg, 97.2% yield) as an off white solid. LCMS 305.3 [M+H]⁺.

### Step 4: Synthesis of 4-(1-(4-chlorophenyl)-1H-pyrazol-4-yl)-N-(6-chloroquinolin-2-yl)cyclohexanecarboxamide

To a stirred mixture of 4-(1-(4-chlorophenyl)-1H-pyrazol-4-yl)cyclohexanecarboxylic acid (140 mg, 0.46 mmol, 1.0 equiv), EDCI.HCl (353 mg, 1.84 mmol, 4 equiv) & DMAP (248 mg, 1.84 mmol, 4 equiv) & in DCM (20 mL) was added 6-chloroquinolin-2-amine (82 mg, 0.46 mmol, 1.0 equiv) and the resulting reaction mixture was stirred at RT overnight. The product formation was confirmed by LCMS. After completion of reaction, the reaction mixture was diluted with DCM (50 mL). Organic layer was washed with water (20 mL) and brine (20 mL), dried over anhydrous Na₂SO₄ & concentrated under reduced pressure. The crude product was purified by reversed phase HPLC to obtain 4-(1-(4-chlorophenyl)-1H-pyrazol-4-yl)-N-(6-chloroquinolin-2-yl)cyclohexanecarboxamide (80 mg, 37.5% yield) as a white solid. LCMS 465.5 [M+H]⁺. ¹H NMR (400 MHz, DMSO-d₆) δ 10.77 (s, 1H), 8.41-8.22 (m, 3H), 8.04 (d, J=10.5 Hz, 1H), 7.91-7.81 (m, 2H), 7.80 (d, J=8.3 Hz, 1H), 7.74-7.64 (m, 2H), 7.53 (d, J=8.8 Hz, 2H), 2.84 (br. s., 1H), 2.77 (br. s., 1H), 1.99 (br. s., 2H), 1.81 (br. s., 2H), 1.70 (br. s., 2H), 1.62 (br. s., 2H).

### Step 5: Chiral separation of 4-(1-(4-chlorophenyl)-1H-pyrazol-4-yl)-N-(6-chloroquinolin-2-yl)cyclohexanecarboxamide

The enantiomers, cis-4-(1-(4-chlorophenyl)-1H-pyrazol-4-yl)-N-(6-chloroquinolin-2-yl)cyclohexanecarboxamide (Compound 249—[α]D20=NA; elution time: 6.07 min) and trans-4-(1-(4-chlorophenyl)-1H-pyrazol-4-yl)-N-(6-chloroquinolin-2-yl)cyclohexanecarboxamide (Compound 250—[α]D20=NA; elution time: 9.77 min), were separated by chiral SFC (Chiralcel-OX-H, 250×21 mm, 5p). Isocratic program with analytical grade liquid carbon dioxide, HPLC grade MeOH in 0.2% DEA). LCMS: 464.4 [M+H]⁺.

### Example 64

### Synthesis of 5-chloro-N-(4-(5-(cis-3-(trifluoromethoxy)cyclobutyl)-1,3,4-oxadiazol-2-yl)piperidin-1-yl)benzofuran-2-carboxamide

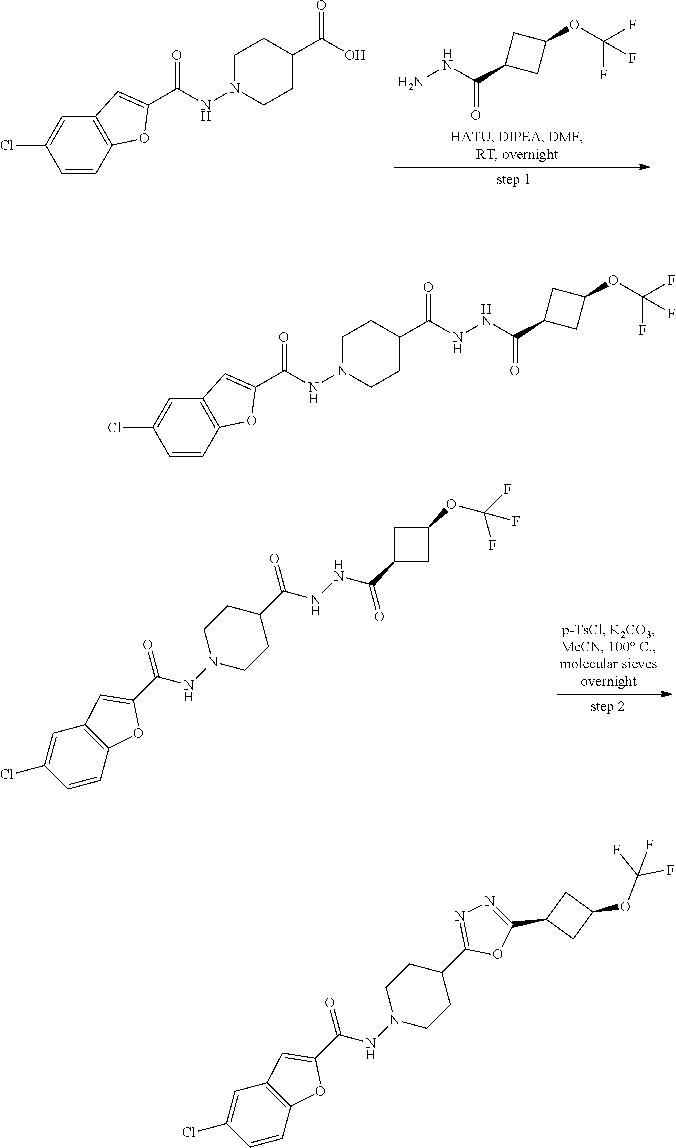 [SMILES: O=C(NN1CCC(C(=O)O)CC1)c1cc2cc(Cl)ccc2o1 ; NNC(=O)[C@H]1C[C@@H](OC(F)(F)F)C1 ; O=C(NN1CCC(C(=O)NNC(=O)[C@H]2C[C@@H](OC(F)(F)F)C2)CC1)c1cc2cc(Cl)ccc2o1 ; O=C(NN1CCC(C(=O)NNC(=O)[C@H]2C[C@@H](OC(F)(F)F)C2)CC1)c1cc2cc(Cl)ccc2o1 ; O=C(NN1CCC(c2nnc([C@H]3C[C@@H](OC(F)(F)F)C3)o2)CC1)c1cc2cc(Cl)ccc2o1]

### Step 1: Synthesis of 5-chloro-N-(4-(2-(cis-3-(trifluoromethoxy)cyclobutanecarbonyl)hydrazinecarbonyl)piperidin-1-yl)benzofuran-2-carboxamide

To a stirred mixture of 1-(5-chlorobenzofuran-2-carboxamido)piperidine-4-carboxylic acid (100 mg, 0.310 mmol, 1.0 equiv) cis-3-(trifluoromethoxy)cyclobutanecarbohydrazide (63 mg, 0.372 mmol, 1.2 equiv) and HATU (235 mg, 0.62 mmol, 2.0 equiv) in DMF (0.5 mL) was added DIPEA (79 mg, 0.62 mmol, 2.0 equiv). The resulting reaction mixture was stirred at RT overnight. Product formation was confirmed by LCMS. After completion of reaction, the reaction mixture was poured into ice cold water (10 ml). The resulting solid was filtered off and dried under vacuum to obtain 5-chloro-N-(4-(2-(cis-3-(trifluoromethoxy)cyclobutanecarbonyl)hydrazinecarbonyl)piperidin-1-yl)benzofuran-2-carboxamide (100 mg, 64.5% yield) as an off white solid. LCMS 503.1 [M+H]⁺. ¹H NMR (400 MHz, DMSO-d₆) δ 9.86 (br. s., 3H) 9.79 (d, J=14.91 Hz, 1H) 7.86-7.70 (d, J=9.21 Hz, 1H) 7.37-7.56 (m, 2H) 4.73-4.87 (m, 2H) 3.02 (br. s., 2H) 2.89 (s, 1H) 2.56-2.83 (m, 4H) 2.16-2.41 (m, 3H) 1.73 (br. s., 3H).

### Step 2: Synthesis of 5-chloro-N-(4-(5-(cis-3-(trifluoromethoxy)cyclobutyl)-1,3,4-oxadiazol-2-yl)piperidin-1-yl)benzofuran-2-carboxamide

To a stirred solution of 5-chloro-N-(4-(2-(cis-3-(trifluoromethoxy)cyclobutanecarbonyl)hydrazinecarbonyl)piperidin-1-yl)benzofuran-2-carboxamide (100 mg, 0.199 mmol, 1.0 equiv), K₂CO₃ (137 mg, 0.996 mmol, 5 equiv) and molecular sieves (100 mg) in acetonitrile (10 mL) was added 4-toluenesulfonyl chloride (94 mg, 0.497 mmol, 2.5 equiv) and the resulting reaction mixture was stirred at 100° C. overnight. Product formation was confirmed by LCMS and TLC. After completion of reaction, the reaction mixture filtered through Celite® and filtrate was diluted with EtOAc (50 mL). Organic layer was washed with water (25 mL) and brine (25 mL), dried over anhydrous Na₂SO₄ and concentrated. The crude product was purified by reverse phase purification to obtain 5-chloro-N-(4-(5-(cis-3-(trifluoromethoxy)cyclobutyl)-1,3,4-oxadiazol-2-yl)piperidin-1-yl)benzofuran-2-carboxamide (Compound 251-10 mg, 10.3% yield) as an white solid. LCMS 485.5 [M+H]⁺. ¹H NMR (400 MHz, DMSO-d₆) δ 9.88 (s, 1H) 7.87 (d, J=2.19 Hz, 1H) 7.71 (s, 1H) 7.32-7.59 (m, 2H) 4.90-5.00 (m, 1H) 3.38-3.51 (m, 2H) 3.17 (d, J=5.26 Hz, 1H) 3.06 (d, J=11.40 Hz, 2H) 2.99 (br. s., 1H) 2.77-2.94 (m, 3H) 2.74-2.67 (d, J=6.58 Hz, 1H) 2.03-2.19 (m, 2H) 1.75-2.03 (m, 2H).

### Example 65

### Synthesis of 6-chloro-N-(4-(5-((1s,3s)-3-(trifluoromethoxy)cyclobutyl)-1,3,4-oxadiazol-2-yl)piperidin-1-yl)-3,4-dihydro-2H-benzo[b][1,4]oxazine-2-carboxamide

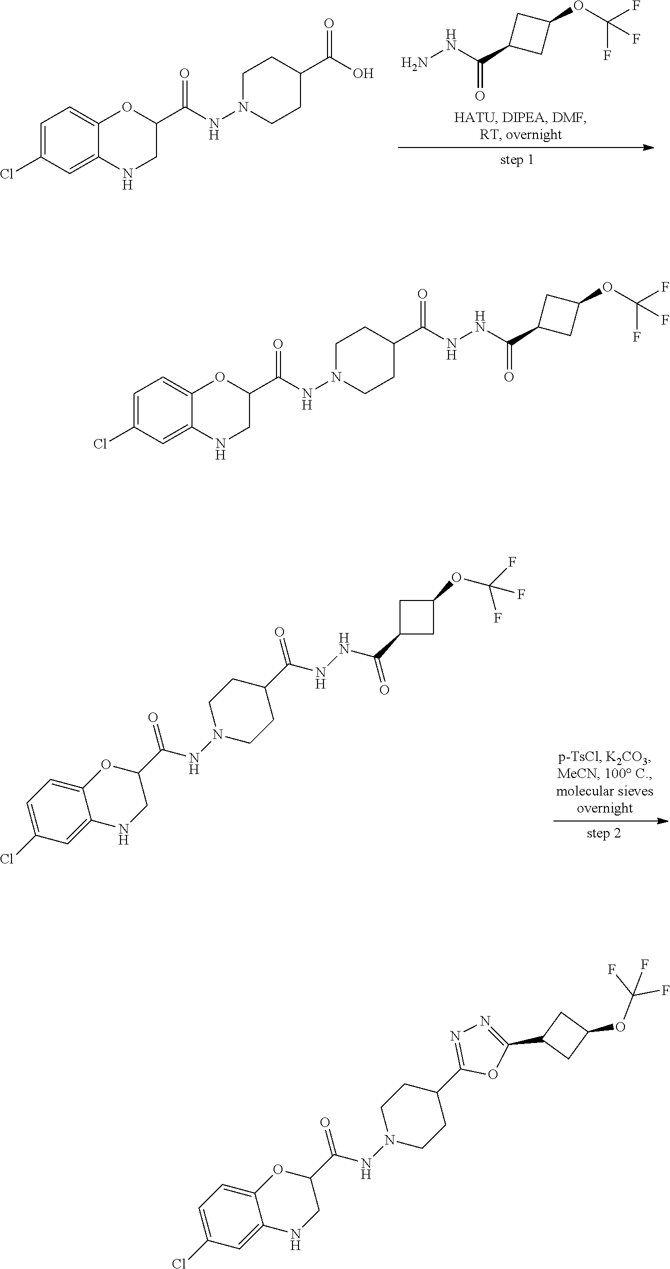

### Step 1: Synthesis of 6-chloro-N-(4-(2-((1s,3s)-3-(trifluoromethoxy)cyclobutanecarbonyl)hydrazinecarbonyl)piperidin-1-yl)-3,4-dihydro-2H-benzo[b][1,4]oxazine-2-carboxamide

To a stirred mixture of 1-(6-chloro-3,4-dihydro-2H-benzo[b][1,4]oxazine-2-carboxamido)piperidine-4-carboxylic acid (70 mg, 0.206 mmol, 1.0 equiv) (1s,3s)-3-(trifluoromethoxy)cyclobutanecarbohydrazide (49 mg, 0.247 mmol, 1.2 equiv) and HATU (156 mg, 0.412 mmol, 2.0 equiv) in DMF (0.5 mL) was added DIPEA (53 mg, 0.412 mmol, 2.0 equiv). The resulting reaction mixture was stirred at RT for overnight. Product formation was confirmed by LCMS. After completion of reaction, the reaction mixture was poured into ice cold water (10 ml). The resulting off white solid was filtered off and dried under vacuum to obtain 6-chloro-N-(4-(2-((1s,3s)-3-(trifluoromethoxy)cyclobutanecarbonyl)hydrazinecarbonyl)piperidin-1-yl)-3,4-dihydro-2H-benzo[b][1,4]oxazine-2-carboxamide (80 mg, 74.7% Yield) as an off white solid. LCMS 520.2 [M+H]⁺. ¹H NMR (400 MHz, DMSO-d₆) δ9.85 (br. s., 1H) 9.66-9.80 (m, 1H) 9.04 (s, 1H) 6.76 (d, J=8.58 Hz, 1H) 6.50-6.64 (m, 1H) 6.42-6.50 (m, 1H) 6.18 (br. s., 1H) 4.74-4.84 (m, 1H) 4.40 (dd, J=7.15, 2.86 Hz, 1H) 3.41 (d, J=11.92 Hz, 2H) 3.05-3.27 (m, 2H) 2.83-3.05 (m, 2H) 2.57-2.76 (m, 4H) 2.19-2.40 (m, 2H) 2.15 (d, J=10.49 Hz, 1H) 1.68 (br. s., 3H).

### Step 2: Synthesis of 6-chloro-N-(4-(5-((1s,3s)-3-(trifluoromethoxy)cyclobutyl)-1,3,4-oxadiazol-2-yl)piperidin-1-yl)-3,4-dihydro-2H-benzo[b][1,4]oxazine-2-carboxamide

To a stirred solution of 6-chloro-N-(4-(2-((1s,3s)-3-(trifluoromethoxy)cyclobutanecarbonyl)hydrazinecarbonyl)piperidin-1-yl)-3,4-dihydro-2H-benzo[b][1,4]oxazine-2-carboxamide (50 mg, 0.096 mmol, 1.0 equiv), K₂CO₃ (66 mg, 0.481 mmol, 5.0 equiv) and molecular sieves (50 mg) in acetonitrile (10 mL) was added 4-toluenesulfonyl chloride (45 mg, 0.24 mmol, 2.5 equiv) and the resulting reaction mixture was stirred at 100° C. for overnight. Product formation was confirmed by LCMS and TLC. After completion of reaction, the reaction mixture filtered through Celite® and filtrate was diluted with EtOAc (50 mL). Organic layer was washed with water (25 mL) and brine (25 mL), dried over anhydrous Na₂SO₄ and concentrated. The crude product was purified by reversed phase HPLC to obtain 6-chloro-N-(4-(5-((1s,3s)-3-(trifluoromethoxy)cyclobutyl)-1,3,4-oxadiazol-2-yl)piperidin-1-yl)-3,4-dihydro-2H-benzo[b][1,4]oxazine-2-carboxamide (Compound 252-10 mg, 20.8% Yield) as an off white solid. LCMS 502.6 [M+H]⁺. ¹H NMR (400 MHz, DMSO-d₆) δ 9.11 (s, 1H) 6.76 (d, J=8.58 Hz, 1H) 6.38-6.65 (m, 2H) 6.18 (br. s., 1H) 4.90 (quin, J=7.39 Hz, 2H) 4.41 (dd, J=7.39, 2.62 Hz, 2H) 3.39-3.53 (m, 2H) 3.07-3.25 (m, 2H) 2.74-2.97 (m, 5H) 1.92-2.21 (m, 3H) 1.67-1.92 (m, 2H).

### Example 66

### Synthesis of (2R,4R)-6-chloro-N-((trans-4-(5-(4-chlorophenyl)-1,3,4-oxadiazol-2-yl)cyclohexyl)methyl)-4-hydroxychroman-2-carboxamide

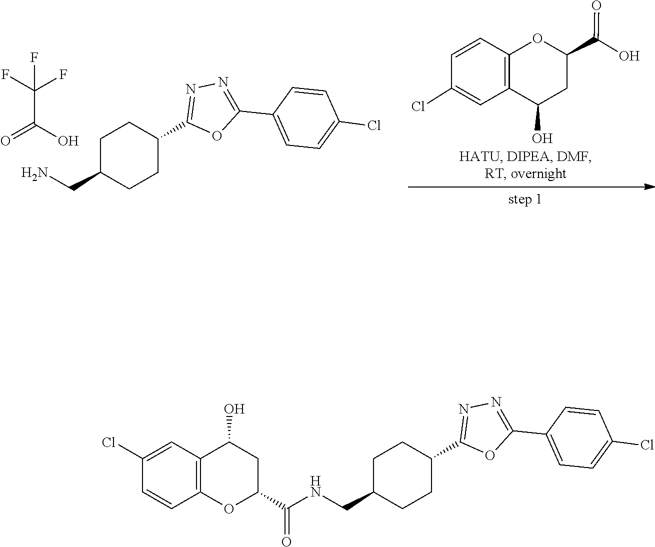

To a stirred solution of trans-(4-(5-(4-chlorophenyl)-1,3,4-oxadiazol-2-yl)cyclohexyl)methanamine 2,2,2-trifluoroacetate (50 mg, 0.1234 mmol, 1.0 equiv), (2R,4R)-6-chloro-4-hydroxychroman-2-carboxylic acid (28 mg, 0.1234 mmol, 1.0 equiv) & HATU (71 mg, 0.185 mmol, 1.5 equiv) in DMF (1 mL) was added DIPEA (0.09 mL, 0.379 mmol, 4.0 equiv). The resulting reaction mixture was stirred at RT overnight. Product formation was confirmed by LCMS. After completion of reaction, the reaction mixture was poured into ice cold water (10 ml) and extracted with ethyl acetate (2×10 mL). Organic layer was washed with water (4×10 mL), brine solution (1×10 mL), dried over anhydrous Na₂SO₄ and concentrated under reduced pressure. The crude product was purified by flash chromatography (0-5% MeOH in DCM as an eluent) to obtain (2R,4R)-6-chloro-N-((trans-4-(5-(4-chlorophenyl)-1,3,4-oxadiazol-2-yl)cyclohexyl)methyl)-4-hydroxychroman-2-carboxamide (Compound 253-10 mg, 16.2% yield) as an off white solid. LCMS 502.5 [M+H]⁺. ¹H NMR (400 MHz, DMSO-d₆) δ 8.14 (t, J=6.0 Hz, 1H), 8.04-7.85 (m, J=8.6 Hz, 2H), 7.74-7.58 (m, J=8.6 Hz, 2H), 7.39 (d, J=1.9 Hz, 1H), 7.20 (dd, J=2.9, 8.6 Hz, 1H), 6.92-6.77 (m, 1H), 5.70 (d, J=6.2 Hz, 1H), 4.87-4.77 (m, 1H), 4.67 (dd, J=2.4, 11.9 Hz, 1H), 3.05 (t, J=6.4 Hz, 2H), 2.97 (t, J=12.2 Hz, 1H), 2.33 (s, 1H), 2.16 (d, J=11.9 Hz, 2H), 1.87-1.66 (m, 3H), 1.62-1.43 (m, 3H), 1.17-0.96 (m, 2H).

### Example 67

### Synthesis of 5-chloro-N-(4-(5-(cis-3-(trifluoromethyl)cyclobutyl)-1,3,4-oxadiazol-2-yl)piperidin-1-yl)benzofuran-2-carboxamide

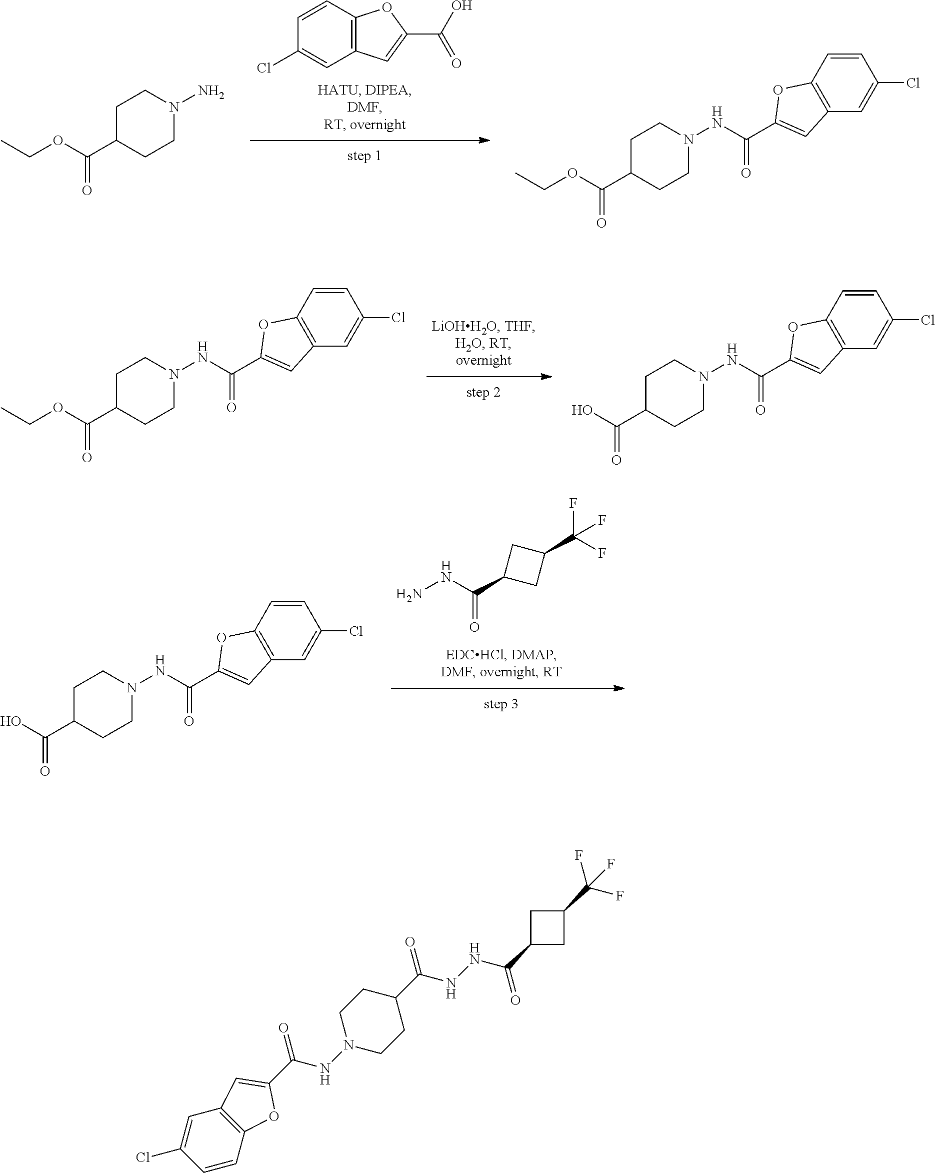

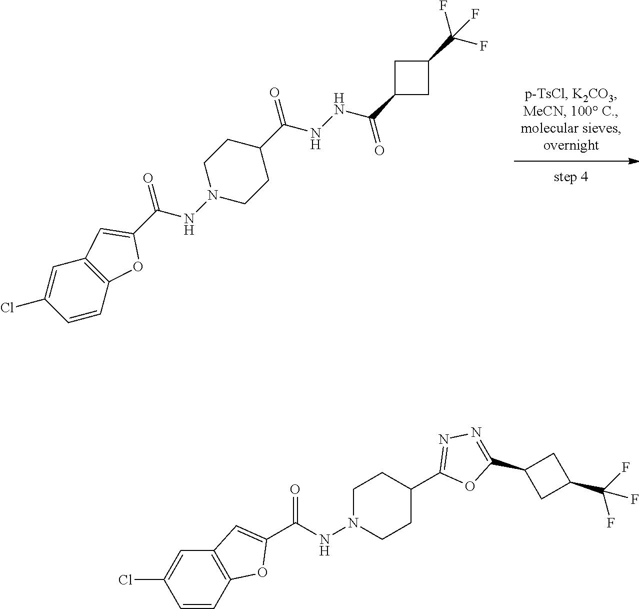 [SMILES: O=C(NN1CCC(C(=O)NNC(=O)[C@H]2C[C@@H](C(F)(F)F)C2)CC1)c1cc2cc(Cl)ccc2o1 ; O=C(NN1CCC(c2nnc([C@H]3C[C@@H](C(F)(F)F)C3)o2)CC1)c1cc2cc(Cl)ccc2o1]

### Step 1: Synthesis of ethyl 1-(5-chlorobenzofuran-2-carboxamido)piperidine-4-carboxylate

To a stirred solution of ethyl 1-aminopiperidine-4-carboxylate (100 mg, 0.581 mmols, 1.0 equiv), 5-chlorobenzofuran-2-carboxylic acid (150 mg, 0.872 mmols, 1.0 equiv) and HATU (497 mg, 1.308 mmol, 1.5 equiv) in DMF (5 mL) was added DIPEA (0.5 mL, 2.62 mmols, 3.0 equiv). The resulting reaction mixture was stirred at RT overnight. Product formation was confirmed by LCMS. After completion of reaction, the reaction mixture was poured into ice cold water (10 ml). The resulting solid was filtered off and dried under vacuum to obtain ethyl 1-(5-chlorobenzofuran-2-carboxamido)piperidine-4-carboxylate (100 mg, 32.7% yield) as an off white solid. LCMS 351.2 [M+H]⁺. ¹H NMR (400 MHz, DMSO-d₆) δ 9.82 (s, 1H), 7.86 (d, J=1.9 Hz, 1H), 7.69 (d, J=8.6 Hz, 1H), 7.53-7.31 (m, 2H), 4.08 (q, J=7.0 Hz, 2H), 2.98 (d, J=10.5 Hz, 2H), 2.84-2.73 (m, 2H), 2.37-2.24 (m, 1H), 1.88 (d, J=11.0 Hz, 2H), 1.70 (q, J=10.5 Hz, 2H), 1.26-1.04 (m, 9H).

### Step 2: Synthesis of 1-(5-chlorobenzofuran-2-carboxamido)piperidine-4-carboxylic acid

To as stirred solution of ethyl 1-(5-chlorobenzofuran-2-carboxamido)piperidine-4-carboxylate (100 mg, 0.558 mmols, 1.0 equiv) in THF:Water (2:2 mL) was added LiOH.H₂O (18 mg, 0.429 mmols, 1.5 equiv) and allowed to stir for overnight at RT. Product formation was confirmed by LCMS. The reaction mixture was concentrated under reduced pressure. The resulting residue was diluted with water and acidified with 1 M HCl (pH˜3 to 4). The resulting solid was filtered off and dried under vacuum to obtain 1-(5-chlorobenzofuran-2-carboxamido)piperidine-4-carboxylic acid (70 mg, 76% yield) as an off white solid. LCMS 323.2 [M+H]⁺. ¹H NMR (400 MHz, DMSO-d₆) δ 12.23 (br. s., 1H), 9.80 (s, 1H), 7.86 (s, 1H), 7.69 (d, J=8.6 Hz, 1H), 7.58-7.39 (m, 2H), 2.97 (br. s., 2H), 2.76 (t, J=9.8 Hz, 2H), 2.25 (br. s., 1H), 1.87 (d, J=11.9 Hz, 2H), 1.69 (d, J=9.5 Hz, 2H).

### Step 3: Synthesis of 5-chloro-N-(4-(2-(cis-3-(trifluoromethyl)cyclobutanecarbonyl)hydrazinecarbonyl)piperidin-1-yl)benzofuran-2-carboxamide

To a stirred mixture of 1-(5-chlorobenzofuran-2-carboxamido)piperidine-4-carboxylic acid (70 mg, 0.217 mmol, 1.0 equiv), HATU (124 mg, 0.326 mmol, 1.5 equiv) & cis-3-(trifluoromethyl)cyclobutanecarbohydrazide (43 mg, 0.239 mmol, 1.1 equiv) & in DMF (2 mL) was added DIPEA (0.12 mg, 0.652 mmol, 3.0 equiv). The resulting reaction mixture was stirred at RT overnight. The product formation was confirmed by LCMS. After completion of reaction, the reaction mixture was poured into ice cold water (20 ml). The resulting solid was filtered off and dried under vacuum to obtain 5-chloro-N-(4-(2-(cis-3-(trifluoromethyl)cyclobutanecarbonyl)hydrazinecarbonyl)piperidin-1-yl)benzofuran-2-carboxamide (100 mg, 94.6% yield) as an off white solid. LCMS 487.2[M+H]⁺.

### Step 4: Synthesis of 5-chloro-N-(4-(5-(cis-3-(trifluoromethyl)cyclobutyl)-1,3,4-oxadiazol-2-yl)piperidin-1-yl)benzofuran-2-carboxamide

To a stirred mixture of 5-chloro-N-(4-(2-(cis-3-(trifluoromethyl)cyclobutanecarbonyl)hydrazinecarbonyl)piperidin-1-yl)benzofuran-2-carboxamide (100 mg, 0.206 mmol, 1.0 equiv), K₂CO₃ (142 mg, 1.03 mmol, 5.0 equiv) & molecular sieves (200 mg) in acetonitrile (15 mL) was added 4-toluenesulfonyl chloride (98 mg, 0.514 mmol, 2.5 equiv) and the resultant reaction mixture was stirred at 100° C. overnight. The product formation was confirmed by LCMS and TLC. Reaction mixture was filtered and the filtrate was concentrated under reduced pressure. The residue was dissolved in with EtOAc (50 mL). Organic layer was washed with water (20 mL) & brine (20 mL), dried over anhydrous Na₂SO₄ and concentrated. The crude product was purified by flash chromatography (0-5% MeOH in DCM as an eluent) to obtain 5-chloro-N-(4-(5-(cis-3-(trifluoromethyl)cyclobutyl)-1,3,4-oxadiazol-2-yl)piperidin-1-yl)benzofuran-2-carboxamide (Compound 257-10 mg, 10.5% yield) as an off white solid. LCMS 469.2 [M+H]⁺. ¹H NMR (400 MHz, DMSO-d₆) δ 9.88 (s, 1H), 7.87 (s, 1H), 7.70 (d, J=8.6 Hz, 1H), 7.56-7.39 (m, 2H), 3.79 (quin, J=8.8 Hz, 1H), 3.15-2.96 (m, 3H), 2.88 (t, J=9.8 Hz, 2H), 2.70-2.57 (m, 3H), 2.40-2.25 (m, 2H), 2.07 (d, J=11.4 Hz, 2H), 1.92-1.74 (m, 2H).

### Example 68

### Synthesis of 2-(4-chloro-3-fluorophenoxy)-N-(4-(5-(cis-3-(trifluoromethyl)cyclobutyl)-1,3,4-oxadiazol-2-yl)piperidin-1-yl)acetamide

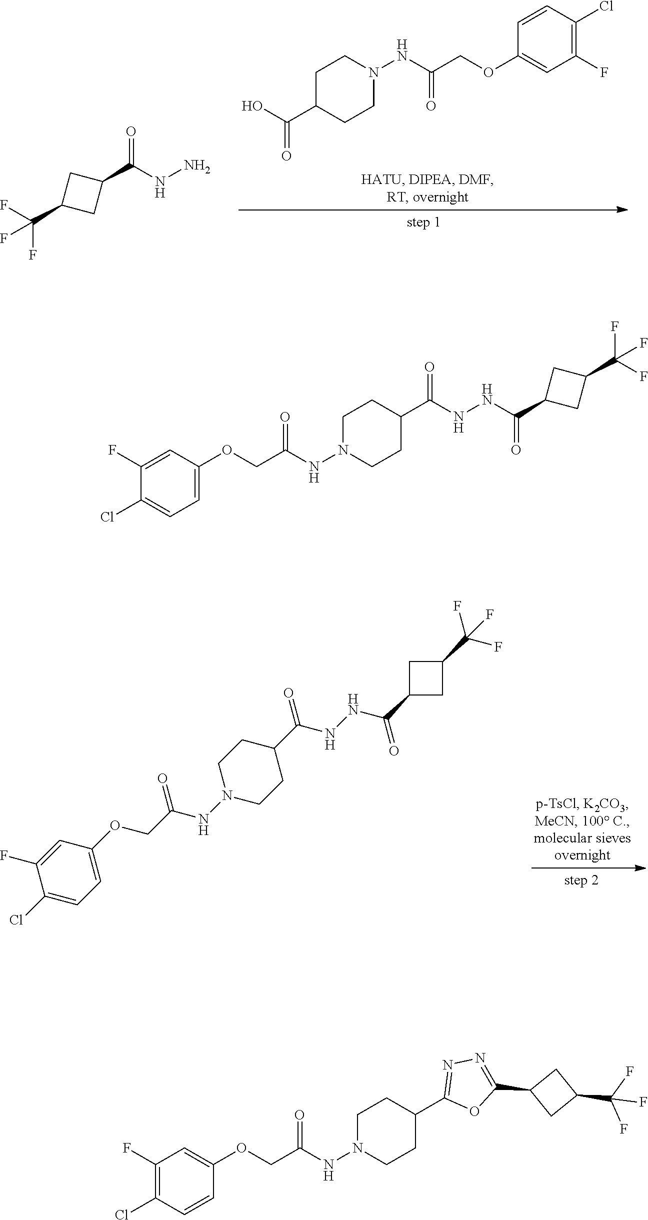 [SMILES: NNC(=O)[C@H]1C[C@@H](C(F)(F)F)C1 ; O=C(COc1ccc(Cl)c(F)c1)NN1CCC(C(=O)O)CC1 ; O=C(COc1ccc(Cl)c(F)c1)NN1CCC(C(=O)NNC(=O)[C@H]2C[C@@H](C(F)(F)F)C2)CC1 ; O=C(COc1ccc(Cl)c(F)c1)NN1CCC(C(=O)NNC(=O)[C@H]2C[C@@H](C(F)(F)F)C2)CC1 ; O=C(COc1ccc(Cl)c(F)c1)NN1CCC(c2nnc([C@H]3C[C@@H](C(F)(F)F)C3)o2)CC1]

### Step 1: Synthesis of 2-(4-chloro-3-fluorophenoxy)-N-(4-(2-(cis-3-(trifluoromethyl)cyclobutanecarbonyl)hydrazinecarbonyl)piperidin-1-yl)acetamide

To a stirred mixture of 1-(2-(4-chloro-3-fluorophenoxy)acetamido)piperidine-4-carboxylic acid (100 mg, 0.303 mmol, 1.0 equiv), HATU (230 mg, 0.606 mmol, 2.0 equiv) & cis-3-(trifluoromethyl)cyclobutanecarbohydrazide (110 mg, 0.606 mmol, 2.0 equiv) & in DMF (2 mL) was added DIPEA (0.1 mL, 0.606 mmol, 1.0 equiv). The resulting reaction mixture was stirred at RT overnight. Product formation was confirmed by LCMS and TLC. After completion of reaction, the reaction mixture was poured into ice cold water (10 ml). The resulting solid was filtered off and dried under vacuum to obtain 2-(4-chloro-3-fluorophenoxy)-N-(4-(2-(cis-3-(trifluoromethyl)cyclobutanecarbonyl)hydrazinecarbonyl)piperidin-1-yl)acetamide (135 mg, 66.5% yield) as an off white solid. LCMS 495.2[M+H]⁺.

### Step 2: Synthesis of 2-(4-chloro-3-fluorophenoxy)-N-(4-(5-(cis-3-(trifluoromethyl)cyclobutyl)-1,3,4-oxadiazol-2-yl)piperidin-1-yl)acetamide

To a stirred mixture of 2-(4-chloro-3-fluorophenoxy)-N-(4-(2-(cis-3-(trifluoromethyl)cyclobutanecarbonyl)hydrazinecarbonyl)piperidin-1-yl)acetamide (135 mg, 0.273 mmol, 1.0 equiv), K₂CO₃ (188 mg, 1.365 mmol, 5.0 equiv) & molecular sieves (1000 mg) in acetonitrile (30 mL) was added 4-toluenesulfonyl chloride (129 mg, 0.683 mmol, 2.5 equiv) and the resultant reaction mixture was stirred at 100° C. overnight. The product formation was confirmed by LCMS and TLC. Reaction mixture was filtered, concentrated under vacuum and the residue obtained was diluted with EtOAc (50 mL). Organic extracts were washed with water (20 mL) & brine (20 mL), dried over anhydrous Na₂SO₄ and concentrated. The crude product was purified by flash chromatography (5% MeOH in DCM as an eluent) to obtain 2-(4-chloro-3-fluorophenoxy)-N-(4-(5-(cis-3-(trifluoromethyl)cyclobutyl)-1,3,4-oxadiazol-2-yl)piperidin-1-yl)acetamide (Compound 259-15 mg, 11.5% yield) as an off white solid. LCMS 477.1 [M+H]⁺. ¹H NMR (400 MHz, DMSO-d₆) δ 9.23 (s, 0.5H), 8.88 (s, 0.5H), 7.51-7.37 (m, 1H), 7.12-6.99 (m, 1H), 6.86-6.69 (m, 1H), 4.91 (s, 1H), 4.48 (s, 1H), 3.77 (quin, J=8.9 Hz, 1H), 3.13 (br. s., 1H), 2.94 (d, J=11.0 Hz, 2H), 2.80-2.70 (m, 1H), 2.67 (s, 1H), 2.65-2.54 (m, 4H), 2.33 (br. s., 1H), 2.01 (br. s., 2H), 1.87-1.65 (m, 2H).

### BIOLOGICAL EXAMPLES

### Example B1-ATF4 Expression Inhibition Assay

The ATF4 reporter was prepared by fusing the human full length 5′UTR of ATF4 (NCBI Accession No. BC022088.2) upstream of the firefly luciferase coding sequence lacking the initiator methionine. The fused sequence was cloned into pLenti-EF1a-C-Myc-DDK-IRES-Puro cloning vector (Origen #PS100085) using standard methods. Virus production was carried out by using Lenti-X™ Packaging Single Shots Protocol (Clonetech #631276). Viral particles were used to transduce HEK293T cells (ATCC #CRL-3216, ATCC Manassas, Va.), which were subsequently selected with puromycin to generate stable cell line. Cells were maintained at 37° C. and 5% CO₂ in DMEM-F12 (Hyclone #SH30023.02) supplemented with 10% heat-inactivated fetal bovine serum (Gibco #16000-044), 2 mM L-glutamine (Gibco #25030-081), 100 U/ml penicillin, and 100 μg/ml streptomycin (Gibco #15140-122).

HEK293T cells carrying the ATF4 luciferase reporter were plated on 96-well plates (Nunc) at 10,000 cells per well. Cells were treated two days after seeding with 100 nM thapsigargin (Tg) (Sigma-Aldrich #T9033) in the presence of different concentrations of selected compounds ranging from 0.1 nM to 10 μM. Cells without treatment or cells treated with Tg alone were used as controls. Assay plates containing cells were incubated for 3 hours at 37° C.

Luciferase reactions were performed using Luciferase Assay System (Promega #E1501) as specified by the manufacturer. Luminescence was read with an integration time of 1 s and a gain of 110 using a Cytation-5 multi-mode microplate reader (BioTek). Relative luminescence units were normalized to Tg treatment (0% inhibition) and untreated cells (100% inhibition) and the percentage of ATF4 inhibition was calculated.

The half-maximal inhibitory concentration (IC₅₀) for the increasing of ATF4 protein levels is shown in Table 6. Under ISR stressed conditions (resulting from treatment with Tg), ATF4 expression is generally upregulated. Accordingly, inhibition of ATF4 expression as a result of the test compound indicates suppression of the ISR pathway.

The activity of the tested compounds is provided in Table 6 as follows: +++=IC50<10 nM; ++=IC50 10-100 nM; +=IC50 100-1000 nM; †=IC50>1000 nM

TABLE 6

| Compound No. | ATF4 inhibition |
|---|---|
| 1 | † |
| 2 | + |
| 3 | ++ |
| 4 | † |
| 5 | +++ |
| 6 | +++ |
| 7 | +++ |
| 8 | +++ |
| 9 | ++ |
| 10 | +++ |
| 11 | ++ |
| 12 | +++ |
| 13 | +++ |
| 14 | +++ |
| 15 | ++ |
| 16 | † |
| 17 | +++ |
| 18 | +++ |
| 19 | ++ |
| 20 | + |
| 21 | + |
| 28 | + |
| 29 | † |
| 30 (first eluting diastereoisomer | + |
| of Example 30) | |
| 31 (second eluting diastereoisomer | † |
| of Example 30) | |
| 32 | † |
| 33 | † |
| 34 | +++ |
| 35 | +++ |
| 36 (first eluting diastereoisomer | + |
| of Step 5 of Example 35) | |
| 37 (second eluting diastereoisomer | +++ |
| of Step 5 of Example 35) | |
| 38 | + |
| 39 | † |
| 40 | ++ |
| 41 | + |
| 42 (first eluting diastereoisomer | +++ |
| of Example 40) | |
| 43 (second eluting diastereoisomer | +++ |
| of Example 40 | |
| 44 | +++ |
| 45 | ++ |
| 46 | ++ |
| 47 | † |
| 48 | ++ |
| 49 | + |
| 50 | + |
| 51 | + |
| 52 | ++ |
| 53 | + |
| 54 | + |
| 237 | ++ |
| 238 | +++ |
| 239 | +++ |
| 240 | +++ |
| 241 | + |
| 242 | +++ |
| 243 | ++ |
| 244 | +++ |
| 245 (first eluting diastereoisomer | + |
| of Example 60) | |
| 246 (second eluting diastereoisomer | + |
| of Example 60) | |
| 247 | +++ |
| 248 | + |
| 249 (first eluting diastereoisomer | + |
| of Step 5 of Example 63) | |
| 250 (second eluting diastereoisomer | ++ |
| of Step 5 of Example 63) | |
| 251 | +++ |
| 252 | +++ |
| 253 | + |

### Example B2—Protein Synthesis Assay

Chinese hamster ovary (CHO) cells were maintained at 37° C. and 5% CO₂ in Dulbecco's Modified Eagle's Media (DMEM) supplemented with 10% fetal bovine serum, 2 mM L-glutamine, 100 U/ml penicillin, and 100 μg/ml streptomycin. After reaching 80% of confluence, cells were detached and seeded on 6 well plates in complete media, allowed to recover overnight and treated for 2 hours with 1 μM of the test compound (to assess protein synthesis levels in unstressed condition), or for 2 hours with 300 nM Tg in the presence of 1 μM of the test compound (to assess the recovery of protein synthesis in a stressed condition). Cells with Tg alone were used as controls.

After the 2 hours treatments, media were replaced by adding 10 μg/ml puromycin (Sigma Aldrich #P8833) in complete media for 30 min. Media were removed and cells were lysed with SDS-PAGE lysis buffer. Lysates were transferred to 1.5 ml tubes and sonicated for 3 min and total protein amount were quantified using BCA Protein Assay Kit (Pierce). Equal amount of protein (30 μg) was loaded on SDS-PAGE gels. Proteins were transferred onto 0.2 μm PVDF membranes (BioRad) and probed with primary antibodies diluted in Tris-buffered saline supplemented with 0.1% Tween 20 (Merck #S6996184 505) and 3% bovine serum albumin (Rockland #BSA-50).

Puromycin (12D10) (Merck #MABE343) and 3-actin (Sigma Aldrich #A5441) antibodies were used as primary antibody. A HRP-conjugated secondary antibody (Rockland) was employed to detect immune-reactive bands using enhanced chemiluminescence (ECL Western Blotting Substrate, Pierce). Quantification of protein bands was done by densitometry using ImageJ software.

Percent increase of protein synthesis in unstressed cells (without Tg treatment) in the presence of media alone or certain test compounds is shown in Table 7. The percentage levels were normalized to the media alone condition, which correspond to 100% protein synthesis. Certain compounds stimulated protein synthesis above baseline, indicating that these test compounds result in increased protein synthesis in unstressed cells.

Percent recovery of protein synthesis in stressed cells (with Tg treatment) due to the test compounds at 1 μM is also shown in Table 7. The levels were normalized to the media alone and Tg alone conditions, which correspond to 10000 and 000 respectively.

TABLE 7

| | % Protein | |
|---|---|---|
| | synthesis | % Recovery |
| | relative | of protein |
| | to untreated | synthesis |
| Compound | (1 μM test | (1 μM test |
| No. | compound) | compound) |
| 1 | 158.0 | 173.2 |
| 2 | 136.3 | 68.5 |
| 3 | 137.6 | 83.0 |
| 4 | 122.7 | 43.2 |
| 5 | 139.1 | 53.7 |
| 6 | 110.7 | −14.0 |
| 7 | 80.9 | 22.3 |
| 8 | 96.9 | 7.2 |
| 9 | 203.7 | 120.8 |
| 10 | 164.9 | 78.6 |
| 11 | 140.1 | 68.4 |
| 12 | 144 | 52.5 |
| 13 | 133.8 | 44.1 |
| 14 | 176.2 | 101.3 |
| 15 | 103.3 | −66.6 |
| 16 | 114.7 | −11.4 |
| 17 | 105 | 47.1 |
| 18 | 126.7 | 76.3 |
| 19 | 100.5 | −1 |
| 28 | 23.8 | 105.5 |
| 29 | 11.5 | 121.5 |
| 30 (first eluting diastereoisomer | 16.7 | 128.6 |
| of Example 30) | | |
| 31 (second eluting diastereoisomer | 4.9 | 103.1 |
| of Example 30) | | |
| 32 | 136 | 148.3 |
| 33 | 11.4 | 66.3 |
| 34 | 83.4 | 75.3 |
| 35 | 51.6 | 113.6 |
| 36 (first eluting diastereoisomer | 6.4 | 110.3 |
| of Step 5 of Example 35) | | |
| 37 (second eluting diastereoisomer | 24.3 | 101.8 |
| of Step 5 of Example 35) | | |
| 42 (first eluting diastereoisomer | 79.9 | 115.9 |
| of Example 40) | | |
| 43 (second eluting diastereoisomer | 86.3 | 140.7 |
| of Example 40) | | |
| 44 | 39.5 | 92.9 |
| 45 | 81.8 | 117.9 |
| 46 | 11.8 | 77.9 |
| 47 | 2 | 80.5 |
| 48 | 22.4 | 72.5 |
| 237 | 51.3 | 125.9 |
| 238 | 74.6 | 93 |
| 239 | 197.2 | 175.6 |
| 240 | 42.3 | 99.7 |
| 242 | 23 | 94.9 |
| 243 | −39.1 | 102.8 |
| 244 | 94 | 88.7 |
| 247 | 277.8 | 94.2 |
| 250 (second eluting diastereoisomer | 25 | 136.2 |
| of Step 5 of Example 63) | | |

Data summarized in Tables 6 and 7 show that some compounds have differential activity in ATF4 inhibition and protein synthesis under ISR-inducing conditions. That is, some compounds are able to effectively inhibit ATF4 expression but do not restore protein synthesis. Other compounds effectively restore protein synthesis but do not inhibit ATF4 expression under ISR-inducing conditions. Still other compounds inhibit ATF4 expression and restore protein synthesis. This differential modulation of activities represents a unique characteristic that can be exploited when selecting specific compounds for a desired use.

### Example B3—ATF4 Inhibition Assay Under Aβ Stimulation

N2A cells are maintained at 37° C. and 5% CO₂ in DMEM-F12 media supplemented with 10% fetal bovine serum (FBS), penicillin and streptomycin. After reaching 80% of confluence, cells are detached and seeded on 6 well plates in complete media, allowed to recover 48 h and treated for 16 hours with 30 μM of purified Aβ₍₁₋₄₂₎ (Abcam) in the presence of 1 μM of selected compounds.

After 16 hours treatment, culture media are removed and cells are lysed with SDS-PAGE lysis buffer. Lysates are transferred to 1.5 ml tubes and sonicated for 3 min. Total protein amount is quantified using BCA Protein Assay Kit (Pierce). Equal amount of proteins (30 μg) is loaded on SDS-PAGE gels. Proteins are transferred onto 0.2 μm PVDF membranes (BioRad) and probed with primary antibodies diluted in Tris-buffered saline supplemented with 0.1% Tween 20 and 3% bovine serum albumin.

ATF4 (11815) antibody is used as primary antibody (Cell Signaling Technologies). A β-actin antibody is used as a control primary antibody. An HRP-conjugated secondary antibody (Rockland) is employed to detect immune-reactive bands using enhanced chemiluminescence (ECL Western Blotting Substrate, Pierce). Quantification of protein bands is done by densitometry using ImageJ.

Percent inhibition of ATF4 expression in N2A cells after incubation with purified Aβ₍₁₋₄₂₎ as a result of the test compounds can be reported. Percentage of ATF4 inhibition is calculated as the percent reduction normalized to the purified Aβ₍₁₋₄₂₎ treatment (0% inhibition) and vehicle treatment (100% inhibition).

### Example B4—Fasting-Induced Muscle Atrophy

Wild type eight-weeks-old male Balb/c mice obtained from the vivarium Fundación Ciencia & Vida Chile (Santiago, Chile) are used. Mice are housed in independent plastic cages in a room maintained at 25° C. with a 12-h:12-h light:dark cycle.

Twenty-four hours before and during the 2 days of fasted procedures, animals receive oral administration via feeding tubes (15 gauge) of vehicle (50% Polyethylene glycol 400 (Sigma-Aldrich P3265) in distilled water or 10 mg/kg of test compound formulated in vehicle solution.

After 2 days of fasting the animals are sacrificed and muscles are removed from both hindlimbs. Mice with feed and water ad libitum are used as control.

During muscle atrophy, protein synthesis is reduced and protein degradation is increased as known in the art. For in vivo measurements of protein synthesis, puromycin (Sigma-Aldrich, P8833) is prepared at 0.04 μmol/g body weight in a volume of 200 μL of PBS, and subsequently administered into the animals via IP injection, 30 min prior to muscle collection.

Upon collection, muscles are immediately frozen in liquid nitrogen and then stored at −80° C. The frozen muscles are then homogenized with a T 10 basic ULTRA-TURRAX (IKa) in ice-cold buffer lysis (Cell Signaling 9803) and protease and phosphatase inhibitors (Roche). Lysates are sonicated for 3 min and centrifuged at 13,000 rpm for 20 minutes at 4° C. Protein concentration in supernatants is determined using BCA Protein Assay Kit (Pierce). Equal amount of proteins is loaded on SDS-PAGE gels. Proteins are transferred onto 0.2 um PVDF membranes (BioRad) and probed with primary antibodies diluted in Tris-buffered saline supplemented with 0.1% Tween 20 and 3% bovine serum albumin.

Puromycin (12D10) (Merck Millipore), MuRF-1 (Santa Cruz Biotechnology) and β-actin (Sigma-Aldrich) antibodies are used as primary antibodies. A HIRP-conjugated secondary antibody (Rockland) is employed to detect immune-reactive bands using enhanced chemiluminescence (ECL Western Blotting Substrate, Pierce). Quantification of protein bands is done by densitometry using ImageJ software.

For immunohistochemical analysis of cross-sectional area (CSA), muscles from control (Fed) and fasted animals are submerged individually in optimal cutting temperature (OCT) compound (Tissue-Tek; Sakura) at resting length, and frozen in isopentane cooled with liquid nitrogen. Cross-sections (10-μm thick) from the mid-belly of the muscles are obtained with a cryostat (Leica) and immunostained with puromycin antibody (12D10) (Merck Millipore). A HRP-polymer conjugated secondary antibody (Biocare Medical, MM620L) followed by diaminobenzidine substrate incubation (ImmPACT DAB-Vector, SK-4105) are employed to detect puromycinylated structures in CSA.

Percent of protein synthesis in quadriceps, gastrocnemius and tibialis anterior of each mouse from fed or fasted animals treated with vehicle or with test compounds can be visualized. The levels are normalized to p-actin expression and percentage is calculated as the percent relative to protein synthesis levels from control mice (Fed) which correspond to 100%.

Muscle fiber CSA are visualized with a Zeiss Axio Lab.A1 microscope and an Axiocam (Zeiss) digital camera. Puromycin staining in CSA can be reported.

Expression of the muscle atrophy marker MuRF-1 in quadriceps from fed or fasted mice treated with vehicle or with test compounds can be visualized. The levels are normalized to p-actin expression and fold change is calculated as the level relative to MuRF-1 levels from control mice (Fed) which correspond to 1.

### Example B5—ISR-Related Pancreatitis Model

Pancreatitis induced by cerulein is the most widely used experimental animal model of acute pancreatitis (See. Hyun et al.; Clin Endosc. 2014 May; 47(3): 212-216) and it was demonstrated that the ISR pathway is highly involved (Sci Transl Med. 2020 Jan. 8; 12 (525):eaay5186).

Wild type eight-weeks-old male C57Bl/6 mice obtained from the vivarium Fundación Ciencia & Vida Chile (Santiago, Chile) are used. Mice are housed in independent plastic cages in a room maintained at 25° C. with a 12-h:12-h light:dark cycle.

Acute pancreatitis is induced by administration of seven hourly intraperitoneal injections of cerulein (50 μg/kg) (Tocris Bioscience), whereas mice in the control group are injected with saline as described by Hernandez et al. in Sci. Transl. Med. 2020 Jan. 8; 12 (525), eaay5186. 18 hours and 2 hours before cerulein injection, animals are orally dosed with 10 mg/kg of test compound. 4 hours and 24 hours after the first injection of cerulein, animals are sacrificed and pancreas are collected to assess the expression of the ISR-related ATF3 transcription factor.

The expression of ATF3 in pancreas is assessed by IHC in formalin-fixed paraffin embedded pancreas using an anti-ATF3 primary antibody (Sigma-Aldrich). A HRP-conjugated polymer (Biocare) and a Diaminobenzidine substrate are used for ATF3 detection.

ATF3 expression is also assessed by Western blot in frozen pancreas. ATF3 (Cell Signaling) and p-actin (Sigma-Aldrich) antibodies are used as primary antibodies. A HRP-conjugated secondary antibody (Rockland) is employed to detect immune-reactive bands using enhanced chemiluminescence (ECL Western Blotting Substrate, Pierce).

### Example B6—Protein Synthesis in a Cell-Free System

The expression of the green fluorescence protein (GFP) was evaluated using the 1-Step Human In vitro Protein Expression Kit based on HeLa cell lysates (ThermoFisher Scientific). HeLa lysate, accessory proteins, reaction mix and pCFE-GFP plasmid from the kit were thawed in ice. Reactions were prepared at room temperature in a 96-well optical plate by adding 12.5 μL of HeLa lysate, 2.5 μL accessory proteins, 5 μL reaction mix, 1 μg of pCFE-GFP plasmid and 1 μM of test compounds in 5 μL distilled H₂0 or 5 μL of distilled H₂0 as a basal expression of GFP (vehicle). A well with dH₂O instead of pCFE-GFP plasmid was used as basal autofluorescence of the reaction. All reactions were made in duplicate. Fluorescence intensity was measured by a multi-mode microplate reader (Synergy-4; Biotek) during 5-hour treatments and capturing fluorescence at 15-minute intervals with 485/20 and 528/20 excitation and emission filters.

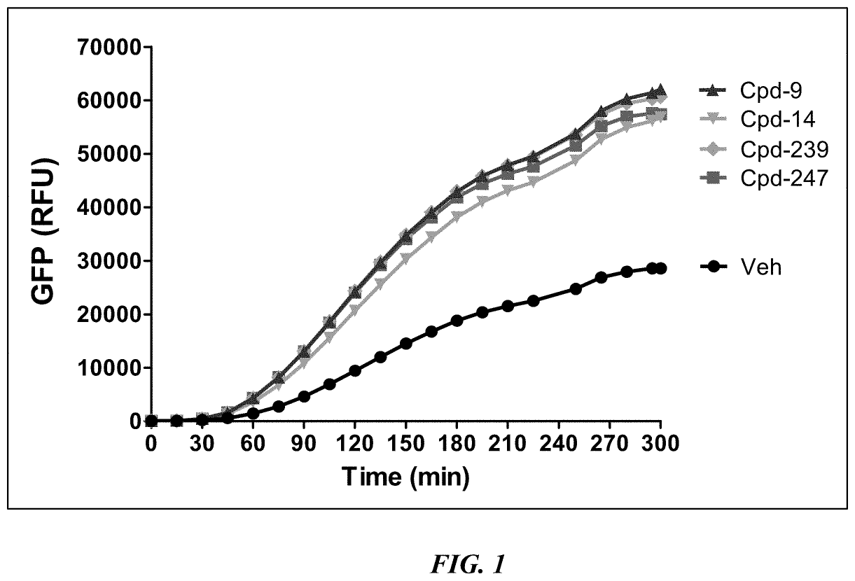

Relative fluorescence intensity (RFU) of GFP treated with either vehicle or test compounds is shown in FIG. 1. The addition of tested compounds to the kit's reaction mix increased the expression of GFP and hence its fluorescence compared to the expression obtained using the kit's reagents alone.

All references throughout, such as publications, patents, patent applications and published patent applications, are incorporated herein by reference in their entireties.

1. A compound of formula (I)
A-L¹-L²-L³-B-D-L⁴-E (I)
or a pharmaceutically acceptable salt thereof,
wherein:
A is selected from the group consisting of:
C₁₀-C₁₄ aryl optionally substituted with 1, 2, 3, 4, 5, 6, 7, 8, or 9 R^{A} substituents;
9-14 membered heteroaryl optionally substituted with 1, 2, 3, 4, 5, 6, 7, 8, or 9 R^{A} substituents; and
8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
wherein R^{L1} is H, C₁-C₆ alkyl, or C₁-C₆ haloalkyl;
wherein L¹ is optionally further substituted by 1, 2, 3, 4, 5, or 6 substituents selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
wherein R^{L2} is H, C₁-C₆ alkyl, or C₁-C₆ haloalkyl;
L³ is a bond, —N(R^{L3})—, or —CH₂—;
wherein R^{L3} is H, C₁-C₆ alkyl, or C₁-C₆ haloalkyl;
B is selected from the group consisting of: 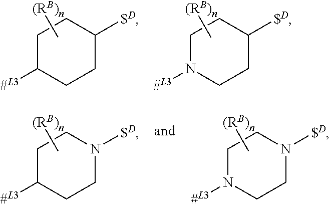
wherein
#^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
C₄-C₁₄ cycloalkyl optionally substituted with 1, 2, 3, 4, 5, 6, 7, 8, or 9 R^{E} substituents;
3-14 membered heterocycloalkyl optionally substituted with 1, 2, 3, 4, 5, 6, 7, 8, or 9 R^{E} substituents;
C₆-C₁₄ aryl substituted with 1, 2, 3, 4, 5, 6, 7, 8, or 9 R^{E} substituents;
5-14 membered heteroaryl substituted with 1, 2, 3, 4, 5, 6, 7, 8, or 9 R^{E} substituents; and
8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
n is an integer selected from the group consisting of 0, 1, 2, 3, 4, 5, 6, 7, 8, and 9.

2. The compound of claim 1, or a pharmaceutically acceptable salt thereof, wherein L⁴ is a bond or #^{D}—CH₂—O—$^{E}, wherein #^{D} represents the attachment point to D and $^{E} represents the attachment point to E

3. The compound of claim 1, or the pharmaceutically acceptable salt thereof, wherein B 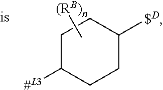
wherein #^{L3} represents the attachment point to L³ and $^{D} represents the attachment point to D.

4. The compound of claim 3, or the pharmaceutically acceptable salt thereof, wherein B 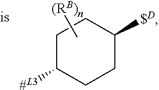
wherein #^{L3} represents the attachment point to L³ and $^{D} represents the attachment point to D.

5. The compound of claim 1, or the pharmaceutically acceptable salt thereof, wherein D is selected from the group consisting of: 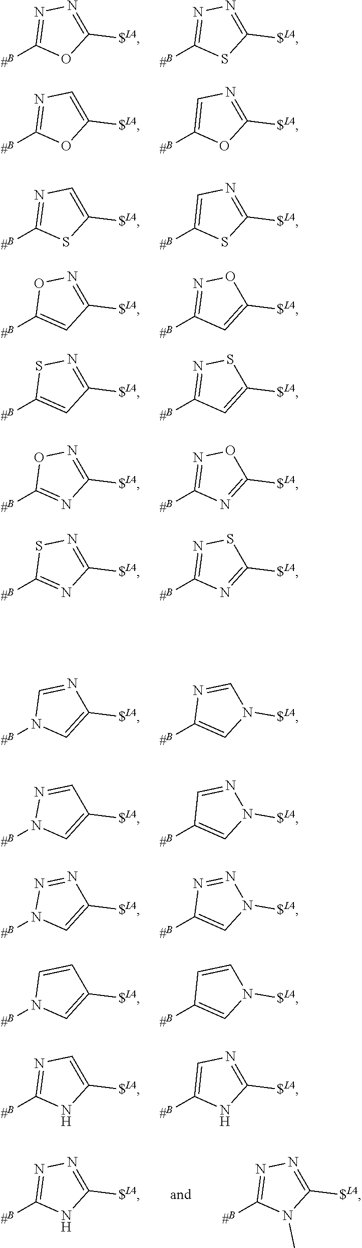
wherein #^{B} represents the attachment point to B and $^{L4} represents the attachment point to L⁴.

6. The compound of claim 1, or the pharmaceutically acceptable salt thereof, wherein D is selected from the group consisting of: 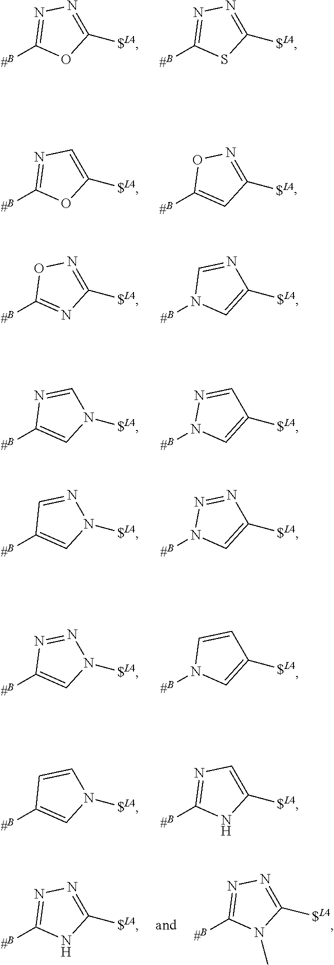
wherein #^{B} represents the attachment point to B and $^{L4} represents the attachment point to L⁴.

7. The compound of claim 1, or the pharmaceutically acceptable salt thereof, wherein D 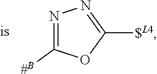
wherein #^{B} represents the attachment point to B and $^{L4} represents the attachment point to L⁴.

8. The compound of claim 1, wherein the compound of formula (I), or the pharmaceutically acceptable salt thereof, is a compound of formula (II): 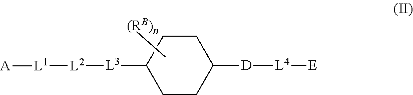
or a pharmaceutically acceptable salt thereof;
wherein D is selected from the group consisting of: 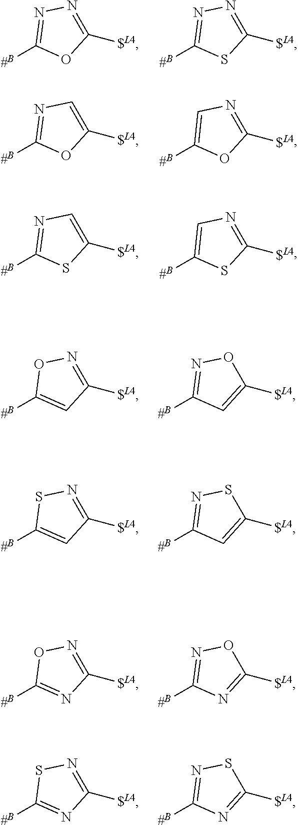 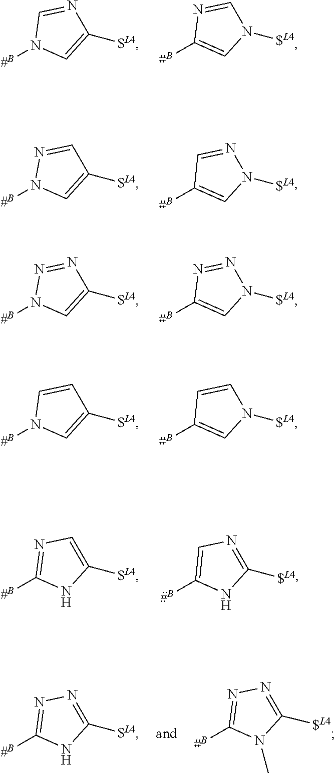
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
provided that when L² is #^{L1}—N(R^{L2})—C(O)—$^{L3}, then L¹ is selected from the group consisting of a bond, #^{A}—(C₁-C₆ alkylene)—$^{L2}, #^{A}—O—$^{L2}, #^{A}—O—(C₁-C₆ alkylene)—$^{L2}, and #^{A}—N(R^{L1})—$^{L2}.

9. The compound of claim 1, wherein the compound of formula (I), or the pharmaceutically acceptable salt thereof, is a compound of formula (II-a): 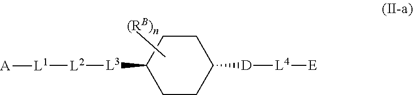
or a pharmaceutically acceptable salt thereof;
wherein D is selected from the group consisting of: 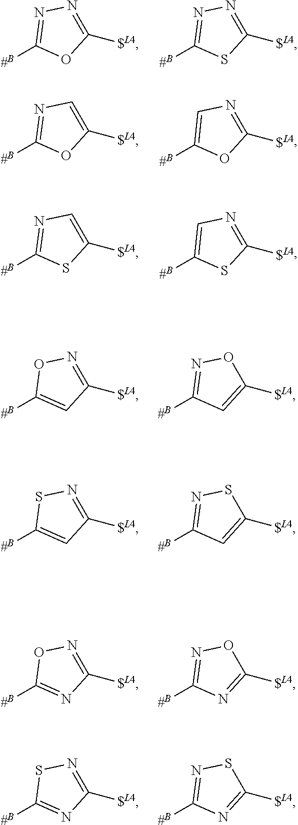 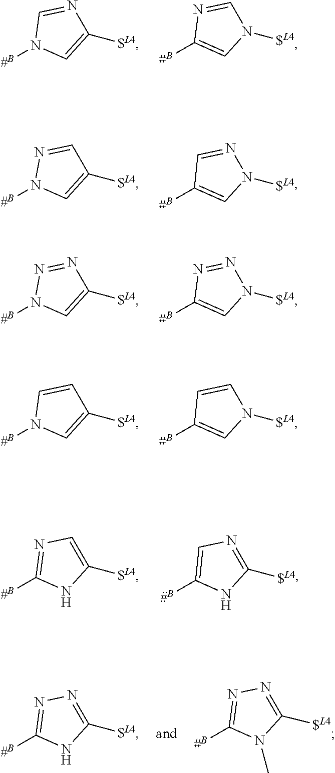
E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl; and
provided that when L² is #^{L1}—N(R^{L2})—C(O)—$^{L3}, then L¹ is selected from the group consisting of a bond, #^{A}—(C₁-C₆ alkylene)—$^{L2}, #^{A}—O—$^{L2}, #^{A}—O—(C₁-C₆ alkylene)—$^{L2}, and #^{A}—N(R^{L1})—$^{L2}.

10. The compound of any one of claims 1-4, wherein the compound of formula (I), or the pharmaceutically acceptable salt thereof, is a compound of formula (III): 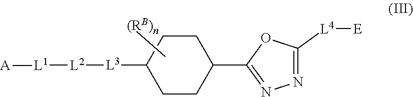
or a pharmaceutically acceptable salt thereof;
wherein E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.

11. The compound of claim 1, wherein the compound of formula (I), or the pharmaceutically acceptable salt thereof, is a compound of formula (III-a): 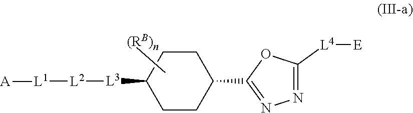
or a pharmaceutically acceptable salt thereof;
wherein E is selected from the group consisting of:
C₆-C₁₄ aryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents;
9-14 membered heteroaryl substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents; and
8-14 membered partially unsaturated fused bicyclic ring moiety substituted with a substituent selected from the group consisting of halogen, C₁-C₆ haloalkyl, O(C₁-C₆ haloalkyl), S(C₁-C₆ haloalkyl), NH(C₁-C₆ haloalkyl), N(C₁-C₆ haloalkyl)₂, C(O)O(C₁-C₆ haloalkyl), C(O)NH(C₁-C₆ haloalkyl), C(O)N(C₁-C₆ haloalkyl)₂, S(O)₂O(C₁-C₆ haloalkyl), S(O)₂NH(C₁-C₆ haloalkyl), S(O)₂N(C₁-C₆ haloalkyl)₂, OC(O)(C₁-C₆ haloalkyl), N(H)C(O)(C₁-C₆ haloalkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ haloalkyl), N(H)S(O)₂(C₁-C₆ haloalkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl), and optionally further substituted with 1, 2, 3, 4, 5, 6, 7, or 8 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.

12. The compound of claim 1, wherein the compound of formula (I), or the pharmaceutically acceptable salt thereof, is a compound of formula (IV): 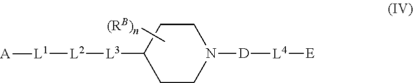
or a pharmaceutically acceptable salt thereof.

13. The compound of claim 1, wherein the compound of formula (I), or the pharmaceutically acceptable salt thereof, is a compound of formula (V): 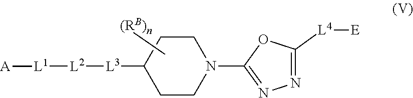
or a pharmaceutically acceptable salt thereof.

14. The compound of claim 1, wherein the compound of formula (I), or the pharmaceutically acceptable salt thereof, is a compound of formula (VI): 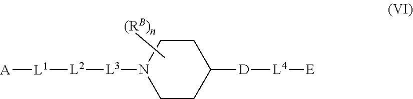
or a pharmaceutically acceptable salt thereof.

15. The compound of claim 1, wherein the compound of formula (I), or the pharmaceutically acceptable salt thereof, is a compound of formula (VII): 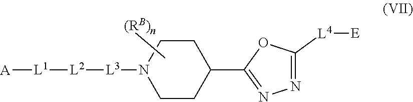
or a pharmaceutically acceptable salt thereof.

16. The compound of claim 1, wherein the compound of formula (I), or the pharmaceutically acceptable salt thereof, is a compound of formula (VIII): 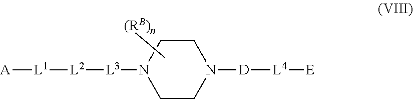
or a pharmaceutically acceptable salt thereof.

17. The compound of claim 1, wherein the compound of formula (I), or the pharmaceutically acceptable salt thereof, is a compound of formula (IX): 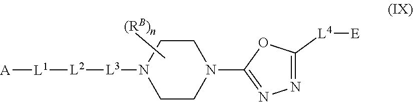
or a pharmaceutically acceptable salt thereof.

18. The compound of claim 1, or the pharmaceutically acceptable salt thereof, wherein L³ is a bond or —CH₂—.

19. The compound of claim 1, or the pharmaceutically acceptable salt thereof, wherein L³ is a bond.

20. The compound of claim 1, or the pharmaceutically acceptable salt thereof, wherein L³ is —CH₂—.

21. The compound of claim 1, or the pharmaceutically acceptable salt thereof, wherein L² is #^{L1}—C(O)—N(R^{L2})—$^{L3}, wherein #^{L1} represents the attachment point to L¹ and $^{L3} represents the attachment point to L³.

22. The compound of claim 21, or the pharmaceutically acceptable salt thereof, wherein L² is #^{L1}—C(O)—NH—$^{L3}, wherein #^{L1} represents the attachment point to L¹ and $^{L3} represents the attachment point to L³.

23. The compound of claim 1, or the pharmaceutically acceptable salt thereof, wherein L² is #^{L1}—N(R^{L2})—C(O)—$^{L3}, wherein #^{L1} represents the attachment point to L¹ and $^{L3} represents the attachment point to L³.

24. The compound of claim 23, or the pharmaceutically acceptable salt thereof, wherein L² is #^{L1}—NH—C(O)—$^{L3}, wherein #^{L1} represents the attachment point to L¹ and $^{L3} represents the attachment point to L³.

25. The compound of claim 1, or the pharmaceutically acceptable salt thereof, wherein L¹ is selected from the group consisting of a bond, #^{A}—CH₂—$^{L2}, #^{A}—C(CH₃)₂—$^{L2}, #^{A}—CH₂—CH₂—$^{L2}, #^{A}O—$^{L2}, #^{A}—O—CH₂—$^{L2}, and #^{A}—N(R^{L1})—$^{L2} wherein #^{A} represents the attachment point to A and $^{L2} represents the attachment point to L².

26. The compound of claim 1, or the pharmaceutically acceptable salt thereof, wherein A is selected from the group consisting of: 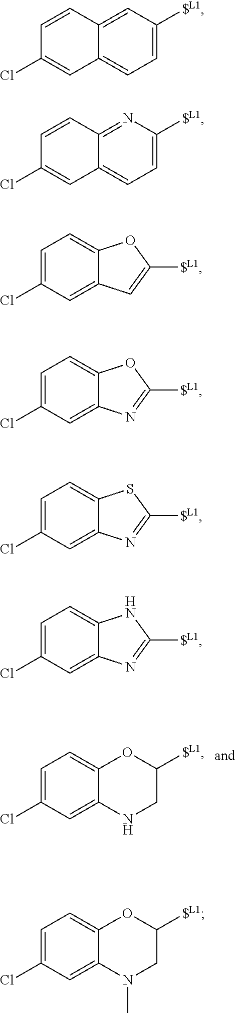
wherein $^{L1} represents the attachment point to L¹.

27. The compound of claim 1, or the pharmaceutically acceptable salt thereof, wherein A is C₁₀-C₁₄ aryl optionally substituted with 1, 2, 3, 4, 5, 6, 7, 8, or 9 R^{A} substituents.

28. The compound of claim 27, or the pharmaceutically acceptable salt thereof, wherein R^{A}, independently at each occurrence, is selected from the group consisting of halogen, C₁-C₆ haloalkyl, and O(C₁-C₆ haloalkyl).

29. The compound of claim 27, or the pharmaceutically acceptable salt thereof, wherein R^{A}, independently at each occurrence, is selected from the group consisting of chloro, fluoro, trifluoromethyl, and trifluoromethoxy.

30. The compound of claim 27, or the pharmaceutically acceptable salt thereof, wherein A is 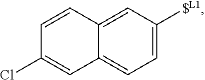
wherein $^{L1} represents the attachment point to L¹.

31. The compound of claim 1, or the pharmaceutically acceptable salt thereof, wherein A is 9-14 membered heteroaryl optionally substituted with 1, 2, 3, 4, 5, 6, 7, 8, or 9 R^{A} substituents.

32. The compound of claim 31, or the pharmaceutically acceptable salt thereof, wherein R^{A}, independently at each occurrence, is selected from the group consisting of halogen and C₁-C₆ haloalkyl.

33. The compound of claim 31, or the pharmaceutically acceptable salt thereof, wherein R^{A}, independently at each occurrence, is selected from the group consisting of chloro, difluoromethyl, and trifluoromethyl.

34. The compound of claim 31, or the pharmaceutically acceptable salt thereof, wherein A is selected from the group consisting of: 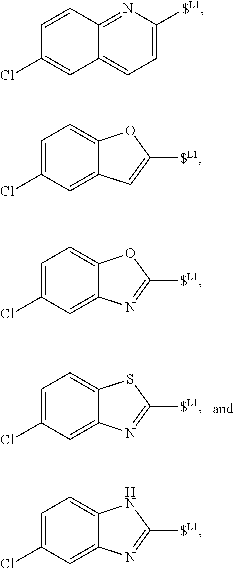
wherein $^{L1} represents the attachment point to L¹.

35. The compound of claim 1, or the pharmaceutically acceptable salt thereof, wherein A is 8-14 membered partially unsaturated fused bicyclic ring moiety, optionally substituted with 1, 2, 3, 4, 5, 6, 7, 8, or 9 R^{A} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.

36. The compound of claim 35, or the pharmaceutically acceptable salt thereof, wherein R^{A}, independently at each occurrence, is selected from the group consisting of halogen and C₁-C₆ alkyl.

37. The compound of claim 35, or the pharmaceutically acceptable salt thereof, wherein R^{A}, independently at each occurrence, is selected from the group consisting of chloro and methyl.

38. The compound of claim 35, or the pharmaceutically acceptable salt thereof, wherein A is selected from the group consisting of: 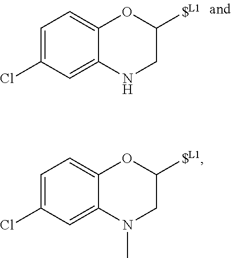
wherein $^{L1} represents the attachment point to L¹.

39. The compound of claim 1, or the pharmaceutically acceptable salt thereof, wherein:
A is 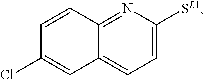
L¹ is a bond, L² is #^{L1}—C(O)—NH—$^{L3}, and L³ is a bond;
A is 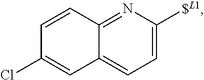
L¹ is a bond, L² is #^{L1}—C(O)—NH—$^{L3}, and L³ is —CH₂—;
A is 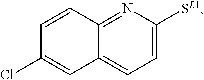
L¹ is a bond, L² is #^{L1}—NH—C(O)—$^{L3}, and L³ is a bond;
A is 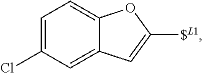
L¹ is a bond, L² is #^{L1}—C(O)—NH—$^{L3}, and L³ is a bond;
A is 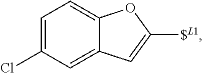
L¹ is a bond, L² is #^{L1}—C(O)—NH—$^{L3}, and L³ is —CH₂—;
A is 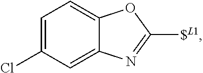
L¹ is a bond, L² is #^{L1}—NH—C(O)—$^{L3}, and L³ is a bond;
A is 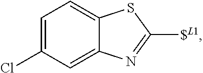
L¹ is a bond, L² is #^{L1}—NH—C(O)—$^{L3}, and L³ is a bond; or
A is 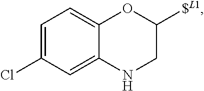
L¹ is a bond, L² is #^{L1}—C(O)—NH—$^{L3}, and L³ is a bond;
wherein $^{L1} represents the attachment point to L¹, #^{A} represents the attachment point to A, $^{L2} represents the attachment point to L², #^{L1} represents the attachment point to L¹, and $^{L3} represents the attachment point to L³.

40. The compound of claim 1, or the pharmaceutically acceptable salt thereof, wherein L⁴ is a bond.

41. The compound of claim 1, or the pharmaceutically acceptable salt thereof, wherein L⁴ is #^{D}—CH₂—O—$^{E}, wherein #^{D} represents the attachment point to D and $^{E} represents the attachment point to E.

42. The compound of claim 1, or the pharmaceutically acceptable salt thereof, wherein E is C₄-C₁₄ cycloalkyl optionally substituted with 1, 2, 3, 4, 5, 6, 7, 8, or 9 R^{E} substituents.

43. The compound of claim 42, or the pharmaceutically acceptable salt thereof, wherein R^{E}, independently at each occurrence, is selected from the group consisting of halogen, C₁-C₆ haloalkyl, OH, O(C₁-C₆ alkyl), and O(C₁-C₆ haloalkyl).

44. The compound of claim 42, or the pharmaceutically acceptable salt thereof, wherein R^{E}, independently at each occurrence, is selected from the group consisting of fluoro, trifluoromethyl, OH, methoxy, and trifluoromethoxy.

45. The compound of claim 42, or the pharmaceutically acceptable salt thereof, wherein E is selected from the group consisting of: 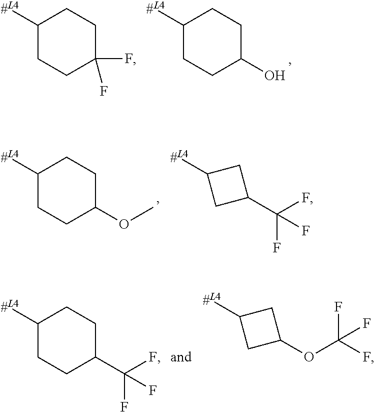
wherein #^{L4} represents the attachment point to L⁴.

46. The compound of claim 1, or the pharmaceutically acceptable salt thereof, wherein E is 3-14 membered heterocycloalkyl optionally substituted with 1, 2, 3, 4, 5, 6, 7, 8, or 9 R^{E} substituents.

47. The compound of claim 46, or the pharmaceutically acceptable salt thereof, wherein R^{E}, independently at each occurrence, is selected from the group consisting of halogen, C₁-C₆ alkyl, C₁-C₆ haloalkyl, OH, O(C₁-C₆ alkyl), and O(C₁-C₆ haloalkyl).

48. The compound of claim 46, or the pharmaceutically acceptable salt thereof, wherein R^{E}, independently at each occurrence, is selected from the group consisting of fluoro, methyl, trifluoromethyl, OH, methoxy, and trifluoromethoxy.

49. The compound of claim 46, or the pharmaceutically acceptable salt thereof, wherein E is selected from the group consisting of: 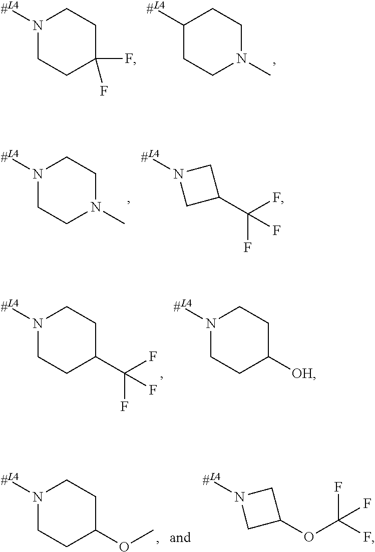
wherein #^{L4} represents the attachment point to L⁴.

50. The compound of claim 1, or the pharmaceutically acceptable salt thereof, wherein E is C₆-C₁₄ aryl optionally substituted with 1, 2, 3, 4, 5, 6, 7, 8, or 9 R^{E} substituents.

51. The compound of claim 50, or the pharmaceutically acceptable salt thereof, wherein R^{E}, independently at each occurrence, is halogen.

52. The compound of claim 50, or the pharmaceutically acceptable salt thereof, wherein R^{E}, independently at each occurrence, is selected from the group consisting of fluoro and chloro.

53. The compound of claim 50, or the pharmaceutically acceptable salt thereof, wherein E is selected from the group consisting of: 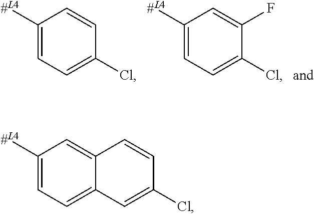
wherein #^{L4} represents the attachment point to L⁴.

54. The compound of claim 1, or the pharmaceutically acceptable salt thereof, wherein E is 5-14 membered heteroaryl optionally substituted with 1, 2, 3, 4, 5, 6, 7, 8, or 9 R^{E} substituents.

55. The compound of claim 54, or the pharmaceutically acceptable salt thereof, wherein R^{E}, independently at each occurrence, is selected from the group consisting of halogen and O(C₁-C₆ alkyl).

56. The compound of claim 54, or the pharmaceutically acceptable salt thereof, wherein R^{E}, independently at each occurrence, is selected from the group consisting of chloro and methoxy.

57. The compound of claim 54, or the pharmaceutically acceptable salt thereof, wherein E is selected from the group consisting of: 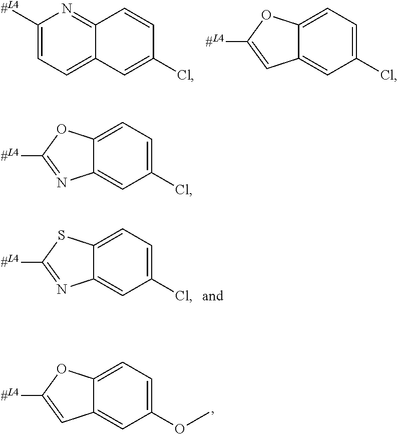
wherein #^{L4} represents the attachment point to L⁴.

58. The compound of claim 1, or the pharmaceutically acceptable salt thereof, wherein E is 8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl.

59. The compound of claim 58, or the pharmaceutically acceptable salt thereof, wherein R^{E}, independently at each occurrence, is halogen.

60. The compound of claim 58, or the pharmaceutically acceptable salt thereof, wherein R^{E}, independently at each occurrence, is chloro.

61. The compound of any claim 58, or the pharmaceutically acceptable salt thereof, wherein E is 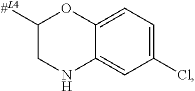
wherein #^{L4} represents the attachment point to L⁴.

62. The compound of claim 1, or the pharmaceutically acceptable salt thereof, wherein:
E is 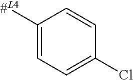
and L⁴ is a bond;
E is 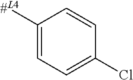
and L⁴ is #^{D}—CH₂—O—$^{E};
E is 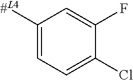
and L⁴ is a bond;
E is 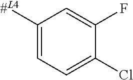
and L⁴ is #^{D}—CH₂—O—$^{E};
E is 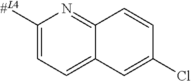
and L⁴ is a bond; or
E is 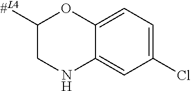
and L⁴ is a bond;
wherein #^{L4} represents the attachment point to L⁴ and #^{D} represents the attachment point to D and $^{E} represents the attachment point to E.

63. The compound of claim 1, or the pharmaceutically acceptable salt thereof, wherein L⁴ and E are taken together to form a group selected from the group consisting of C₁-C₆ haloalkyl, (C₁-C₆ alkylene)—O—(C₁-C₆ haloalkyl), —O—(C₁-C₆ alkylene)—O—(C₁-C₆ haloalkyl), and —NH—(C₁-C₆ alkylene)—O—(C₁-C₆ haloalkyl).

64. The compound of claim 63, or the pharmaceutically acceptable salt thereof, wherein L⁴ and E are taken together to form a group selected from the group consisting of 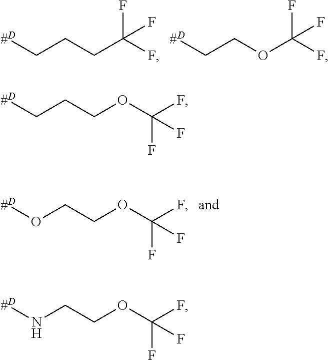
wherein #^{D} represents the attachment point to D.

65. A compound of formula (XIII) 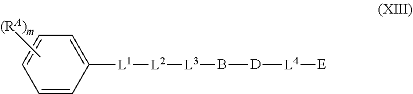
or a pharmaceutically acceptable salt thereof,
wherein:
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
wherein R^{L1} is H, C₁-C₆ alkyl, or C₁-C₆ haloalkyl;
wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
wherein R^{L2} is H, C₁-C₆ alkyl, or C₁-C₆ haloalkyl;
L³ is a bond, —N(R^{L3})—, or —CH₂—;
wherein R^{L3} is H, C₁-C₆ alkyl, or C₁-C₆ haloalkyl;
B is selected from the group consisting of: 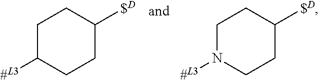
wherein #^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
C₄-C₁₄ cycloalkyl optionally substituted with 1, 2, 3, 4, 5, 6, 7, 8, or 9 R^{E} substituents;
3-14 membered heterocycloalkyl optionally substituted with 1, 2, 3, 4, 5, 6, 7, 8, or 9 R^{E} substituents;
C₆-C₁₄ aryl substituted with a substituent selected from the group consisting of halogen, C₁-C₆ haloalkyl, and O(C₁-C₆ haloalkyl), and optionally further substituted with 1, 2, 3, 4, 5, 6, 7, or 8R^{E} substituents;
5-14 membered heteroaryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents; and
8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
R^{A}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
m is an integer selected from the group consisting of 0, 1, 2, 3, 4, 5, 6, 7, 8, and 9; and
provided that
(i) when B is 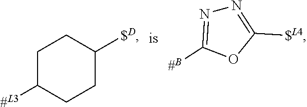
and E is cyclobutyl substituted with 1, 2, 3, 4, 5, 6, 7, 8, or 9 R^{E} substituents, then L⁴ is #^{D}—CH₂—O—$^{E}; or
(ii) when B is 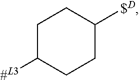
L³ is —CH₂—, and L² is #^{L1}—C(O)—N(R^{L2})—$^{L3}, then L¹ is selected from the group consisting of a bond, C₁-C₆ alkylene, #^{A}—O—(C₁-C₆ alkylene)—$^{L2}, and #^{A}—N(R^{L1})—(C₁-C₆ alkylene)—$^{L2}, wherein #^{A} represents the attachment point to A and $^{L2} represents the attachment point to L².

66. A compound of formula (XVI) 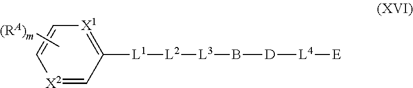
or a pharmaceutically acceptable salt thereof,
wherein:
X¹ and X² are each independently N or CH, provided at least one of X¹ and X² is N;
L¹ is selected from the group consisting of a bond, C₁-C₆ alkylene, #^{A}—O—$^{L2}, #^{A}—O—(C₁-C₆ alkylene)—$^{L2}, #^{A}—(C₁-C₆ alkylene)—O—$^{L2}, #^{A}—N(R^{L1})—$^{L2}, #^{A}—N(R^{L1})—(C₁-C₆ alkylene)—$^{L2}, and #^{A}—(C₁-C₆ alkylene)-N(R^{L1})—$^{L2}, wherein #^{A} represents the attachment point to A and $^{L2} represents the attachment point to L²;
wherein R^{L1} is H, C₁-C₆ alkyl, or C₁-C₆ haloalkyl;
wherein L¹ is optionally further substituted by 1, 2, 3, 4, 5, or 6 substituents selected from the group consisting of halogen, oxo, ═NH, ═N(C₁-C₆ alkyl), ═N(C₁-C₆ haloalkyl), NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
L² is #^{L1}—C(O)—N(R^{L2})—$^{L3} or #^{L1}—N(R^{L2})—C(O)—$^{L3}, wherein #^{L1} represents the attachment point to L¹ and $^{L3} represents the attachment point to L³;
wherein R^{L2} is H, C₁-C₆ alkyl, or C₁-C₆ haloalkyl;
L³ is a bond, —N(R^{L3})—, or —CH₂—;
wherein R^{L3} is H, C₁-C₆ alkyl, or C₁-C₆ haloalkyl;
B is selected from the group consisting of: 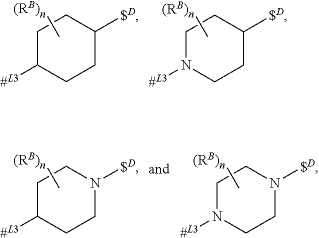
wherein #^{L3} represents the attachment point to L³ and $^{D} represents the attachment point to D;
D is a 5-membered heteroaryl optionally substituted with 1, 2, 3, 4, 5, 6, 7, or 8 R^{D} substituents;
L⁴ is a bond, #^{D}—CH₂—$^{E}, or #^{D}—CH₂—O—$^{E}, wherein #^{D} represents the attachment point to D and $^{E} represents the attachment point to E;
E is selected from the group consisting of:
C₄-C₁₄ cycloalkyl optionally substituted with 1, 2, 3, 4, 5, 6, 7, 8, or 9 R^{E} substituents;
3-14 membered heterocycloalkyl optionally substituted with 1, 2, 3, 4, 5, 6, 7, 8, or 9 R^{E} substituents;
C₆-C₁₄ aryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents;
5-14 membered heteroaryl substituted with a substituent selected from the group consisting of halogen, C₁-C₆ haloalkyl, and O(C₁-C₆ haloalkyl), and optionally further substituted with 1, 2, 3, 4, 5, 6, 7, or 8 R^{E} substituents; and
8-14 membered partially unsaturated fused bicyclic ring moiety, optionally substituted with 1, 2, 3, 4, 5, 6, 7, 8, or 9 R^{E} substituents, wherein the 8-14 membered partially unsaturated fused bicyclic ring moiety comprises a C₅-C₁₀ carbocyclyl fused to a phenyl or a 5-6 membered heteroaryl or a 5-10 membered heterocyclyl fused to a phenyl or a 5-6 membered heteroaryl;
or L⁴ and E are taken together to form a group selected from the group consisting of C₁-C₆ haloalkyl, O(C₁-C₆ haloalkyl), S(C₁-C₆ haloalkyl), NH(C₁-C₆ haloalkyl), (C₁-C₆ alkylene)—O—(C₁-C₆ haloalkyl), (C₁-C₆ alkylene)-S—(C₁-C₆ haloalkyl), (C₁-C₆ alkylene)—NH—(C₁-C₆ haloalkyl), —O—(C₁-C₆ alkylene)—O—(C₁-C₆ haloalkyl), —O—(C₁-C₆ alkylene)-S—(C₁-C₆ haloalkyl), —O—(C₁-C₆ alkylene)—NH—(C₁-C₆ haloalkyl), —S—(C₁-C₆ alkylene)—O—(C₁-C₆ haloalkyl), —S—(C₁-C₆ alkylene)-S—(C₁-C₆ haloalkyl), —S—(C₁-C₆ alkylene)—NH—(C₁-C₆ haloalkyl), —NH—(C₁-C₆ alkylene)—O—(C₁-C₆ haloalkyl), —NH—(C₁-C₆ alkylene)-S—(C₁-C₆ haloalkyl), and —NH—(C₁-C₆ alkylene)—NH—(C₁-C₆ haloalkyl);
R^{A}, independently at each occurrence, is selected from the group consisting of NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{B}, independently at each occurrence, is selected from the group consisting of halogen, oxo, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), N(C₁-C₆ haloalkyl)S(O)₂(C₁-C₆ haloalkyl), C₆-C₁₄ aryl optionally substituted with 1, 2, 3, 4, 5, 6, 7, 8, or 9 R^{BB} substituents, and 5-14 membered heteroaryl optionally substituted with 1, 2, 3, 4, 5, 6, 7, 8, or 9 R^{BB} substituents;
R^{BB}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl);
R^{D}, independently at each occurrence, is selected from the group consisting of oxo, halogen, C₁-C₆ alkyl, C₁-C₆ haloalkyl, —C(O)OH, —C(O)O(C₁-C₆ alkyl), and —C(O)O(C₁-C₆ haloalkyl);
R^{E}, independently at each occurrence, is selected from the group consisting of halogen, NO₂, C₁-C₆ alkyl, C₂-C₆ alkenyl, C₂-C₆ alkynyl, C₁-C₆ haloalkyl, OH, O(C₁-C₆ alkyl), O(C₁-C₆ haloalkyl), SH, S(C₁-C₆ alkyl), S(C₁-C₆ haloalkyl), NH₂, NH(C₁-C₆ alkyl), NH(C₁-C₆ haloalkyl), N(C₁-C₆ alkyl)₂, N(C₁-C₆ haloalkyl)₂, CN, C(O)OH, C(O)O(C₁-C₆ alkyl), C(O)O(C₁-C₆ haloalkyl), C(O)NH₂, C(O)NH(C₁-C₆ alkyl), C(O)NH(C₁-C₆ haloalkyl), C(O)N(C₁-C₆ alkyl)₂, C(O)N(C₁-C₆ haloalkyl)₂, S(O)₂OH, S(O)₂O(C₁-C₆ alkyl), S(O)₂O(C₁-C₆ haloalkyl), S(O)₂NH₂, S(O)₂NH(C₁-C₆ alkyl), S(O)₂NH(C₁-C₆ haloalkyl), S(O)₂N(C₁-C₆ alkyl)₂, S(O)₂N(C₁-C₆ haloalkyl)₂, OC(O)H, OC(O)(C₁-C₆ alkyl), OC(O)(C₁-C₆ haloalkyl), N(H)C(O)H, N(H)C(O)(C₁-C₆ alkyl), N(H)C(O)(C₁-C₆ haloalkyl), N(C₁-C₆ alkyl)C(O)H, N(C₁-C₆ alkyl)C(O)(C₁-C₆ alkyl), N(C₁-C₆ alkyl)C(O)(C₁-C₆ haloalkyl), N(C₁-C₆ haloalkyl)C(O)H, N(C₁-C₆ haloalkyl)C(O)(C₁-C₆ alkyl), N(C₁-C₆ haloalkyl)C(O)(C₁-C₆ haloalkyl), OS(O)₂(C₁-C₆ alkyl), OS(O)₂(C₁-C₆ haloalkyl), N(H)S(O)₂(C₁-C₆ alkyl), N(H)S(O)₂(C₁-C₆ haloalkyl), N(C₁-C₆ alkyl)S(O)₂(C₁-C₆ alkyl), N(C₁-C₆ alkyl)S(O)₂(C₁-C₆ haloalkyl), N(C₁-C₆ haloalkyl)S(O)₂(C₁-C₆ alkyl), and N(C₁-C₆ haloalkyl)S(O)₂(C₁-C₆ haloalkyl); and
m and n are each independently an integer selected from the group consisting of 0, 1, 2, 3, 4, 5, 6, 7, 8, and 9.

67. A compound selected from the group consisting of a compound of Table 1, Table 2, Table 3, Table 4, or Table 5, or a pharmaceutically acceptable salt thereof.

68. A pharmaceutical composition comprising a compound of claim 1, or a pharmaceutically acceptable salt thereof, and a pharmaceutically acceptable carrier.

69. A method of treating a disease or disorder mediated by an integrated stress response (ISR) pathway in an individual in need thereof comprising administering to the individual a therapeutically effective amount of a compound of claim 1, or a pharmaceutically acceptable salt thereof.

70. The method of claim 69, wherein the compound, the pharmaceutically acceptable salt, or the pharmaceutical composition is administered in combination with a therapeutically effective amount of one or more additional anti-cancer agents.

71. The method of claim 69, wherein the disease or disorder is mediated by phosphorylation of eIF2α and/or the guanine nucleotide exchange factor (GEF) activity of eIF2B.

72. The method of claim 69, wherein the disease or disorder is mediated by a decrease in protein synthesis.

73. The method of claim 69, wherein the disease or disorder is mediated by the expression of ATF4, ATF3, CHOP, or BACE-1.

74. The method of claim 69, wherein the disease or disorder is a neurodegenerative disease, an inflammatory disease, an autoimmune disease, a metabolic syndrome, a cancer, a vascular disease, an ocular disease, a musculoskeletal disease, or a genetic disorder.

75. The method of claim 74, wherein the disease is vanishing white matter disease, childhood ataxia with CNS hypomyelination, intellectual disability syndrome, Alzheimer's disease, prion disease, Creutzfeldt-Jakob disease, Parkinson's disease, amyotrophic lateral sclerosis (ALS) disease, cognitive impairment, frontotemporal dementia (FTD), traumatic brain injury, postoperative cognitive dysfunction (PCD), neuro-otological syndromes, hearing loss, Huntington's disease, stroke, chronic traumatic encephalopathy, spinal cord injury, dementias or cognitive impairment, arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis or inflammatory bowel disease, Crohn's disease, ulcerative colitis, celiac disease, systemic lupus erythematosus, type 1 diabetes, multiple sclerosis, rheumatoid arthritis, acute pancreatitis, chronic pancreatitis, alcoholic liver steatosis, obesity, glucose intolerance, insulin resistance, hyperglycemia, fatty liver, dyslipidemia, hyperlipidemia, type 2 diabetes, pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, lung cancer, non-Hodgkin's lymphoma, Burkitt's lymphoma, chronic lymphocytic leukemia, monoclonal gammopathy of undetermined significance (MGUS), plasmocytoma, lymphoplasmacytic lymphoma, acute lymphoblastic leukemia, Pelizaeus-Merzbacher disease, atherosclerosis, abdominal aortic aneurism, carotid artery disease, deep vein thrombosis, Buerger's disease, chronic venous hypertension, vascular calcification, telangiectasia or lymphoedema, glaucoma, age-related macular degeneration, inflammatory retinal disease, retinal vascular disease, diabetic retinopathy, uveitis, rosacea, Sjogren's syndrome or neovascularization in proliferative retinopathy, hyperhomocysteinemia, skeletal muscle atrophy, myopathy, muscular dystrophy, muscular wasting, sarcopenia, Duchenne muscular dystrophy (DMD), Becker's disease, myotonic dystrophy, X-linked dilated cardiomyopathy, spinal muscular atrophy (SMA), Down syndrome, MEHMO syndrome, metaphyseal chondrodysplasia, Schmid type (MCDS), depression, or social behavior impairment.

76. A method of producing a protein, comprising contacting a eukaryotic cell comprising a nucleic acid encoding the protein with a compound of claim 1, or a salt thereof.

77. The method of claim 76, comprising culturing the cell in an in vitro culture medium comprising the compound or salt.

78. A method of culturing a eukaryotic cell comprising a nucleic acid encoding a protein, comprising contacting the eukaryotic cell with an in vitro culture medium comprising a compound of claim 1, or a salt thereof.

79. The method of claim 76, wherein the nucleic acid encoding the protein is a recombinant nucleic acid.

80. The method of claim 76, wherein the cell is a human embryonic kidney (HEK) cell, a Chinese hamster ovary (CHO) cell, or a HeLa cell.

81. The method of claim 76, wherein the cell is a yeast cell, a wheat germ cell, an insect cell, a rabbit reticulocyte, a cervical cancer cell, a baby hamster kidney cell, a murine myeloma cell, an HT-1080 cell, a PER.C6 cell, a hybridoma cell, a human blood derived leukocyte, or a plant cell.

82. A method of producing a protein, comprising contacting a cell-free protein synthesis (CFPS) system comprising eukaryotic initiation factor 2 (eIF2) and a nucleic acid encoding a protein with a compound of claim 1, or a salt thereof.

83. The method of claim 76, wherein the protein is an antibody or a fragment thereof.

84. The method of claim 76, wherein the protein is a recombinant protein, an enzyme, an allergenic peptide, a cytokine, a peptide, a hormone, a growth factor, erythropoietin (EPO), an interferon, a granulocyte-colony stimulating factor (G-CSF), an anticoagulant, or a clotting factor.

85. The method of claim 76, comprising purifying the protein.

86. An in vitro cell culture medium, comprising a compound of claim 1, or a salt thereof.

87. The cell culture medium of claim 86, comprising a eukaryotic cell comprising a nucleic acid encoding a protein.

88. The cell culture medium of claim 86, further comprising a compound for inducing protein expression.

89. The cell culture medium of claim 86, wherein the nucleic acid encoding the protein is a recombinant nucleic acid.

90. The cell culture medium of claim 86, wherein the protein is an antibody or a fragment thereof.

91. The cell culture medium of claim 86, wherein the protein is a recombinant protein, an enzyme, an allergenic peptide, a cytokine, a peptide, a hormone, a growth factor, erythropoietin (EPO), an interferon, a granulocyte-colony stimulating factor (G-CSF), an anticoagulant, or a clotting factor.

92. The cell culture medium of claim 86, wherein the eukaryotic cell is a human embryonic kidney (HEK) cell, a Chinese hamster ovary (CHO) cell, or a HeLa cell.

93. The cell culture medium of claim 86, wherein the cell is a yeast cell, a wheat germ cell, an insect cell, a rabbit reticulocyte, a cervical cancer cell, a baby hamster kidney cell, a murine myeloma cell, an HT-1080 cell, a PER.C6 cell, a hybridoma cell, a human blood derived leukocyte, or a plant cell.

94. A cell-free protein synthesis (CFPS) system comprising eukaryotic initiation factor 2 (eIF2), a nucleic acid encoding a protein, and a compound of claim 1, or a salt thereof.

95. The CFPS system of claim 94, comprising a eukaryotic cell extract comprising eIF2.

96. The CFPS system of claim 94, further comprising eIF2B.

97. The CFPS system of claim 94, wherein the protein is an antibody or a fragment thereof.

98. The CFPS system of claim 94, wherein the protein is a recombinant protein, an enzyme, an allergenic peptide, a cytokine, a peptide, a hormone, a growth factor, erythropoietin (EPO), an interferon, a granulocyte-colony stimulating factor (G-CSF), an anticoagulant, or a clotting factor.

99. A method for enhancing protein synthesis in a living organism, comprising administering to the living organism an effective amount of a compound of claim 1, or a salt thereof.

100. A method for accelerating growth of a plant, comprising administering to the plant an effective amount of a compound of claim 1, or a salt thereof.

101. A method for improving protein yield or quality in a plant, comprising administering to the plant an effective amount of a compound of claim 1, or a salt thereof.

102. The method of claim 101, wherein the plant is selected from the group consisting of soybean, sunflower, grain legume, rice, wheat germ, maize, tobacco, a cereal, and a lupin crop.

103. A compound selected from the group consisting of a compound of Table 1, or a pharmaceutically acceptable salt thereof.
